SEQUENCE LISTING (208 sequences)

SEQ ID NO: 1 - protein, 602 aa, synthetic construct
  REGION 1..602: note = NiVG protein attachment glycoprotein (602 aa)
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 2 - protein, 604 aa, synthetic construct
  REGION 1..604: note = Hendra Virus G Protein
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 3 - protein, 603 aa, synthetic construct
  REGION 1..603: note = Hendra Virus G Protein without Met
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 4 - protein, 602 aa, synthetic construct
  REGION 1..602: note = Nipah Virus G Protein
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 5 - protein, 601 aa, synthetic construct
  REGION 1..601: note = Nipah Virus G Protein (No Met)
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 6 - protein, 622 aa, synthetic construct
  REGION 1..622: note = Cedar Virus G Protein
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 7 - protein, 621 aa, synthetic construct
  REGION 1..621: note = Cedar Virus G Protein (No Met)
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 8 - protein, 632 aa, synthetic construct
  REGION 1..632: note = Bat Paramyxovirus G Protein
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 9 - protein, 631 aa, synthetic construct
  REGION 1..631: note = Bat Paramyxovirus G Protein (No Met)
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 10 - protein, 625 aa, synthetic construct
  REGION 1..625: note = Mojiang virus, Tongguan 1 G Protein
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 11 - protein, 624 aa, synthetic construct
  REGION 1..624: note = Mojiang virus, Tongguan 1 G (No Met)
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 12 - protein, 597 aa, synthetic construct
  REGION 1..597: note = NiVG protein attachment glycoprotein Truncated delta5
MKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 13 - protein, 582 aa, synthetic construct
  REGION 1..582: note = NiVG protein attachment glycoprotein Truncated delta20
MSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 14 - protein, 577 aa, synthetic construct
  REGION 1..577: note = NiVG protein attachment glycoprotein Truncated delta25
MSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 15 - protein, 500 aa, synthetic construct
  REGION 1..500: note = Nipah virus NiV-F F0 T234 truncation (aa 525-544) AND mutation on N-linked glycosylation site
ILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 16 - protein, 524 aa, synthetic construct
  REGION 1..524: note = Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 17 - protein, 569 aa, synthetic construct
  REGION 1..569: note = NiVG protein attachment glycoprotein Truncated and mutated (E501 A, W504A, Q530A, E533A) NiV G protein (Gc delta34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 18 - protein, 568 aa, synthetic construct
  REGION 1..568: note = NiVG protein attachment glycoprotein Truncated and mutated (E501 A, W504A, Q530A, E533A) NiV G protein (Gc delta34) Without N-terminal methionine
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 19 - protein, 524 aa, synthetic construct; identical to SEQ ID NO: 16
  REGION 1..524: note = Truncated NiV fusion glycoprotein (FcDelta22) at cytoplasmic tail (with signal sequence)

SEQ ID NO: 20 - protein, 500 aa, synthetic construct
  REGION 1..500: note = Nipah virus NiV-F F0 T234 truncation (aa 525-544)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 21 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 22 - protein, 552 aa, synthetic construct
  REGION 1..552: note = NivG protein attachment glycoprotein Without cytoplasmic tail Uniprot Q9IH62
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 23 - protein, 604 aa, synthetic construct; identical to SEQ ID NO: 2
  REGION 1..604: note = Hendra virus G protein Uniprot O89343

SEQ ID NO: 24 - protein, 603 aa, synthetic construct; identical to SEQ ID NO: 3
  REGION 1..603: note = Hendra virus G protein Uniprot O89343 Without N-terminal methionine

SEQ ID NO: 25 - protein, 555 aa, synthetic construct
  REGION 1..555: note = Hendra virus G protein Uniprot O89343 Without cytoplasmic tail
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 26 - protein, 555 aa, synthetic construct; identical to SEQ ID NO: 25
  REGION 1..555: note = Hendra virus G protein Uniprot O89343 Without cytoplasmic tail

SEQ ID NO: 27 - protein, 602 aa, synthetic construct; identical to SEQ ID NO: 1
  REGION 1..602: note = NiVG protein attachment glycoprotein (602 aa)

SEQ ID NO: 28 - protein, 546 aa, synthetic construct
  REGION 1..546: note = Hendra virus F Protein
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 29 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 30 - protein, 546 aa, synthetic construct
  REGION 1..546: note = Nipah virus F Protein
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 31 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 32 - protein, 557 aa, synthetic construct
  REGION 1..557: note = Cedar Virus F Protein
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 33 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 34 - protein, 545 aa, synthetic construct
  REGION 1..545: note = Mojiang virus, Tongguan 1 F Protein
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 35 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 36 - protein, 662 aa, synthetic construct
  REGION 1..662: note = Bat Paramyxovirus F Protein
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 37 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 38 - protein, 26 aa, synthetic construct
  REGION 1..26: note = signal sequence
MVVILDKRCYCNLLILILMISECSVG

SEQ ID NO: 39 - protein, 83 aa, synthetic construct
  REGION 1..83: note = Nipah virus NiV-F F2 (aa 27-109)
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 40 - protein, 546 aa, synthetic construct; identical to SEQ ID NO: 30
  REGION 1..546: note = Nipah virus F Protein

SEQ ID NO: 41 - protein, 520 aa, synthetic construct; identical to SEQ ID NO: 31
  REGION 1..520: note = Nipah virus NiV-F F0 (aa 27-546)

SEQ ID NO: 42 - protein, 569 aa, synthetic construct
  REGION 1..569: note = NiVG protein attachment glycoprotein Truncated (Gc delta34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 43 - protein, 573 aa, synthetic construct
  REGION 1..573: note = NiVG protein attachment glycoprotein Truncated delta30
MTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 44 - protein, 592 aa, synthetic construct
  REGION 1..592: note = NiVG protein attachment glycoprotein Truncated delta10
MGNTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 45 - protein, 587 aa, synthetic construct
  REGION 1..587: note = NiVG protein attachment glycoprotein Truncated ?15
MGKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 46 - protein, 437 aa, synthetic construct
  REGION 1..437: note = Nipah virus NiV F F1 (aa 110-546)
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 47 - DNA, 7439 bp, synthetic construct
  misc_feature 1..7439: note = pMA2 CMV-Cre
  source 1..7439: mol_type = other DNA
ggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgcagagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattctcaacaataggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattactcggacagcagagatccagtttggactagtggagttccgcgttacataacttacgctaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatacaagacaccggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagacaagatcacctgcaggacaggcgcgccctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccgggcgattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcacccca

SEQ ID NO: 48 - DNA, 5463 bp, synthetic construct
  misc_feature 1..5463: note = pHAGE with Cre starting at the GAG start codon
  source 1..5463: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgcagagtacgccaaaaattttgactagcggaggctagaaggagagagatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 49 - DNA, 8745 bp, synthetic construct
  misc_feature 1..8745: note = pMA2 CMV-EGFP-IRES-Cre
  source 1..8745: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgcagagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattctcaacaataggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattactcggacagcagagatccagtttggactagtggagttccgcgttacataacttacgctaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatacaagacaccggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagacaagatcacctgcaggacaggcgcgccctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccgggcgattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 50 - DNA, 7429 bp, synthetic construct
  misc_feature 1..7429: note = pMA2 IRES-Cre (no promoter)
  source 1..7429: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgcagagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattctcaacaataggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattactcggacagcagagatccagtttggactaggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagacaagatcacctgcaggacaggcgcgccctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccgggcgattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 51 - DNA, 5880 bp, synthetic construct
  misc_feature 1..5880: note = pMA2 N-term GAG-T2A-Cre (no promoter)
  source 1..5880: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcaggtagtggcgagggcagaggaagtcttctaacatgcggtgacgtggaggagaatcccggccctatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 52 - protein, 117 aa, synthetic construct
  REGION 1..117: note = N-term gag (no Met)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQA

SEQ ID NO: 53 - DNA, 9285 bp, synthetic construct
  misc_feature 1..9285: note = MA-MS2cp-CA gagpol
  source 1..9285: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattctagactgccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggagacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactcccagaactaccccatcgtgcagaacctgcagggccagatggtgcaccaggccatctccccccgcaccctgaacgcctgggtgaaggtggtggaggagaaggccttctcccccgaagtcatccccatgttctccgccctgtccgagggcgccaccccccaggacctgaacaccatgctgaacaccgtgggcggccaccaggccgccatgcagatgctgaaggagaccatcaacgaggaggccgccgagtgggaccgcctgcaccccgtgcacgccggccccatcgcccccggccagatgcgcgagccccgcggctccgacatcgccggcaccacctccaccctgcaagagcagatcggctggatgacccacaacccccccatccccgtgggcgagatctacaagcgctggatcatcctgggcctgaacaagatcgtgcgcatgtactcccccacctccatcctggacatccgccagggccccaaggagcccttccgcgactacgtggaccgcttctacaagaccctgcgcgccgagcaggcctcccaggaggtaaagaactggatgaccgagaccctgctggtgcagaacgccaaccccgactgcaagaccatcctgaaggccctgggccccggcgccaccctggaggagatgatgaccgcctgccagggcgtgggcggccccggccacaaggcccgcgtgctggccgaggccatgtcccaagtcaccaaccccgccaccatcatgatccagaagggcaacttccgcaaccagcgcaagaccgtgaagtgcttcaactgcggcaaggagggccacatcgccaagaactgccgcgccccccgcaagaagggctgctggaagtgcggcaaggagggccaccagatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatcggtggccagctgaaggaggccctgctggacaccggcgccgacgacaccgtgctggaggagatgaacctgcccggccgctggaagcccaagatgatcggcggcatcggcggcttcatcaaagtccgccagtacgaccagatcctgatcgagatctgcggccacaaggccatcggcaccgtgctggtgggccccacccccgtgaacatcatcggccgcaacctgctgacccagatcggctgcaccctgaacttccccatctcccccatcgagaccgtgcccgtgaagctgaagcccggcatggacggccccaaagtcaagcagtggcccctgaccgaggagaagatcaaggccctggtggagatctgcaccgagatggagaaggagggcaagatctccaagatcggccccgagaacccctacaacacccccgtgttcgccatcaagaagaaggactccaccaagtggcgcaagctggtggacttccgcgagctgaacaagcgcacccaggacttctgggaggtgcagctgggcatcccccaccccgccggcctgaagcagaagaagtccgtgaccgtgctggacgtgggcgacgcctacttctccgtgcccctggacaaggacttccgcaagtacaccgccttcaccatcccctccatcaacaacgagacccccggcatccgctaccagtacaacgtgctgccccagggctggaagggctcccccgccatcttccagtgctccatgaccaagatcctggagcccttccgcaagcagaaccccgacatcgtgatctaccagtacatggacgacctgtacgtgggctccgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggactcctggaccgtgaacgacatccagaagctggtgggcaagctgaactgggcctcccagatctacgccggcatcaaagtccgccagctgtgcaagctgctgcgcggcaccaaggccctgaccgaggtggtgcccctgaccgaggaggccgagctggagctggccgagaaccgcgagatcctgaaggagcccgtgcacggcgtgtactacgacccctccaaggacctgatcgccgagatccagaagcagggccagggccagtggacctaccagatctaccaggagcccttcaagaacctgaagaccggcaaatacgcccgcatgaagggcgcccacaccaacgacgtgaagcagctgaccgaggccgtgcagaagatcgccaccgagtccatcgtgatctggggcaagactcccaagttcaagctgcccatccagaaggagacctgggaggcctggtggaccgagtactggcaggccacctggatccccgagtgggagttcgtgaacaccccccccctggtgaagctgtggtaccagctggagaaggagcccatcatcggcgccgagaccttctacgtggacggcgccgccaaccgcgagaccaagctgggcaaggccggctacgtgaccgaccgcggccgccagaaggtggtgcccctgaccgacaccaccaaccagaagaccgagctgcaggccatccacctggccctgcaagactccggcctggaggtgaacatcgtgaccgactcccagtatgcattgggcatcatccaggcccagcccgacaagtccgagtccgagctggtgtcccagatcatcgagcagctgatcaagaaggagaaggtgtacctggcctgggtgcccgcccacaagggcatcggcggcaacgagcaggtggacaagctggtgtccgccggcatccgcaaggtgctgttcctggacggcatcgacaaggcccaggaggagcacgagaagtaccactccaactggcgcgccatggcctccgacttcaacctgccccccgtggtggccaaggagatcgtggcctcctgcgacaagtgccagctgaagggcgaggccatgcacggccaggtggactgctcccccggcatctggcagctggactgcacccacctggagggcaaggtgatcctggtggccgtgcacgtggcctccggctacatcgaggccgaggtgatccccgccgagaccggccaggagaccgcctacttcctgctgaagctggccggccgctggcccgtgaagaccgtgcacaccgacaacggctccaacttcacctccaccaccgtgaaggccgcctgctggtgggccggcatcaagcaggagttcggcatcccctacaacccccagtcccagggcgtgatcgagtccatgaacaaggagctgaagaagatcatcggccaagtccgcgaccaggccgagcacctgaagaccgccgtgcagatggccgtgttcatccacaacttcaagcgcaagggcggcatcggcggctactccgccggcgagcgcatcgtggacatcatcgccaccgacatccagaccaaggagctgcagaagcagatcaccaagatccagaacttccgcgtgtactaccgcgactcccgcgaccccgtgtggaagggccccgccaagctgctgtggaagggcgagggcgccgtggtgatccaggacaactccgacatcaaggtggtgccccgccgcaaggccaagatcatccgcgactacggcaagcagatggccggcgacgactgcgtggcctcccgccaggacgaggactaacacatggaaaagattagtaaaacaccataggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 54 - DNA, 5471 bp, synthetic construct
  misc_feature 1..5471: note = N-term PH domain
  source 1..5471: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggacagcgggagggatttcctcacgctgcacggtttgcaggacgacgaggacctccaagctttgctcaaaggcagtcaacttctcaaggtcaagagcagctcctggaggcgcgaacggttttacaagttgcaagaagattgcaaaacaatttggcaagagagccggaaagtcatgagaactcccgagagccaactgttcagcatcgaggacatccaagaagtcaggatgggccataggaccgagggcttggaaaaattcgccagggacgtgcccgaagaccgatgctttagcatcgtgttcaaagatcagaggaacacgttggacttgatcgcccccagtccggctgatgcccagcattgggttctggggctccataagatcatccatcatagcggcagcatggaccagaggcagaaactccaacattggattcatagttgtcttaggaaagccgacaagaacaaggacaacaagatgagcttcaaggagttgcagaattttcttaaagagctgaatatccagtcccagaactaccccatcgtgcagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 55 - DNA, 5471 bp, synthetic construct
  misc_feature 1..5471: note = C-term PH domain
  source 1..5471: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactcccagaactaccccatcgtgcagatggacagcgggagggatttcctcacgctgcacggtttgcaggacgacgaggacctccaagctttgctcaaaggcagtcaacttctcaaggtcaagagcagctcctggaggcgcgaacggttttacaagttgcaagaagattgcaaaacaatttggcaagagagccggaaagtcatgagaactcccgagagccaactgttcagcatcgaggacatccaagaagtcaggatgggccataggaccgagggcttggaaaaattcgccagggacgtgcccgaagaccgatgctttagcatcgtgttcaaagatcagaggaacacgttggacttgatcgcccccagtccggctgatgcccagcattgggttctggggctccataagatcatccatcatagcggcagcatggaccagaggcagaaactccaacattggattcatagttgtcttaggaaagccgacaagaacaaggacaacaagatgagcttcaaggagttgcagaattttcttaaagagctgaatatccagtaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 56 - DNA, 4994 bp, synthetic construct
  misc_feature 1..4994: note = Myr (n-term; lyn pal-)
  source 1..4994: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgccatcaaatcaaagaggaaggataatctgaatgacgatgaatcccagaactaccccatcgtgcagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 57 - DNA, 5000 bp, synthetic construct
  misc_feature 1..5000: note = Single Pal, from GNA12
  source 1..5000: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgtccggggtggtgcggaccctcagccgctgcctgctgccggccgaggccggctcccagaactaccccatcgtgcagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 58 - DNA, 5012 bp, synthetic construct
  misc_feature 1..5012: note = Double Pal, from GNA13
  source 1..5012: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggcggacttcctgccgtcgcggtccgtgctgtccgtgtgcttccccggctgcctgctgacgagttcccagaactaccccatcgtgcagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 59 - DNA, 5012 bp, synthetic construct
  misc_feature 1..5012: note = Triple Pal from GNA15
  source 1..5012: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggcccgctcgctgacctggcgctgctgcccctggtgcctgacggaggatgagaaggccgccgcctcccagaactaccccatcgtgcagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 60 - DNA, 4994 bp, synthetic construct
  misc_feature 1..4994: note = Myr-pal (lyn), N-term
  source 1..4994: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggctgcatcaaatcaaagaggaaggataatctgaatgacgatgaatcccagaactaccccatcgtgcagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 61 - DNA, 5009 bp, synthetic construct
  misc_feature 1..5009: note = Farnesyl, c-term, from HRAS
  source 1..5009: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactcccagaactaccccatcgtgcagaagctgaaccctcctgatgagagtggccccggctgcatgagctgcaagtgtgtgctctcctgataaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 62 - DNA, 17628 bp, synthetic construct
  misc_feature 1..17628: note = HIV MA (N-term)
  source 1..17628: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggaaacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggacagcgggagggatttcctcacgctgcacggtttgcaggacgacgaggacctccaagctttgctcaaaggcagtcaacttctcaaggtcaagagcagctcctggaggcgcgaacggttttacaagttgcaagaagattgcaaaacaatttggcaagagagccggaaagtcatgagaactcccgagagccaactgttcagcatcgaggacatccaagaagtcaggatgggccataggaccgagggcttggaaaaattcgccagggacgtgcccgaagaccgatgctttagcatcgtgttcaaagatcagaggaacacgttggacttgatcgcccccagtccggctgatgcccagcattgggttctggggctccataagatcatccatcatagcggcagcatggaccagaggcagaaactccaacattggattcatagttgtcttaggaaagccgacaagaacaaggacaacaagatgagcttcaaggagttgcagaattttcttaaagagctgaatatccagtcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggacagcgggagggatttcctcacgctgcacggtttgcaggacgacgaggacctccaagctttgctcaaaggcagtcaacttctcaaggtcaagagcagctcctggaggcgcgaacggttttacaagttgcaagaagattgcaaaacaatttggcaagagagccggaaagtcatgagaactcccgagagccaactgttcagcatcgaggacatccaagaagtcaggatgggccataggaccgagggcttggaaaaattcgccagggacgtgcccgaagaccgatgctttagcatcgtgttcaaagatcagaggaacacgttggacttgatcgcccccagtccggctgatgcccagcattgggttctggggctccataagatcatccatcatagcggcagcatggaccagaggcagaaactccaacattggattcatagttgtcttaggaaagccgacaagaacaaggacaacaagatgagcttcaaggagttgcagaattttcttaaagagctgaatatccagtcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 63 - DNA, 10337 bp, synthetic construct
  misc_feature 1..6149: note = N-term PH domain
  source 1..10337: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattcccagaactaccccatcgtgcagatggacagcgggagggatttcctcacgctgcacggtttgcaggacgacgaggacctccaagctttgctcaaaggcagtcaacttctcaaggtcaagagcagctcctggaggcgcgaacggttttacaagttgcaagaagattgcaaaacaatttggcaagagagccggaaagtcatgagaactcccgagagccaactgttcagcatcgaggacatccaagaagtcaggatgggccataggaccgagggcttggaaaaattcgccagggacgtgcccgaagaccgatgctttagcatcgtgttcaaagatcagaggaacacgttggacttgatcgcccccagtccggctgatgcccagcattgggttctggggctccataagatcatccatcatagcggcagcatggaccagaggcagaaactccaacattggattcatagttgtcttaggaaagccgacaagaacaaggacaacaagatgagcttcaaggagttgcagaattttcttaaagagctgaatatccagtaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggacagcgggagggatttcctcacgctgcacggtttgcaggacgacgaggacctccaagctttgctcaaaggcagtcaacttctcaaggtcaagagcagctcctggaggcgcgaacggttttacaagttgcaagaagattgcaaaacaatttggcaagagagccggaaagtcatgagaactcccgagagccaactgttcagcatcgaggacatccaagaagtcaggatgggccataggaccgagggcttggaaaaattcgccagggacgtgcccgaagaccgatgctttagcatcgtgttcaaagatcagaggaacacgttggacttgatcgcccccagtccggctgatgcccagcattgggttctggggctccataagatcatccatcatagcggcagcatggaccagaggcagaaactccaacattggattcatagttgtcttaggaaagccgacaagaacaaggacaacaagatgagcttcaaggagttgcagaattttcttaaagagctgaatatccagtcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 64 - DNA, 6149 bp, synthetic construct
  source 1..6149: mol_type = other DNA
  misc_feature 1..6149: note = C-term PH domain
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattcccagaactaccccatcgtgcagatggacagcgggagggatttcctcacgctgcacggtttgcaggacgacgaggacctccaagctttgctcaaaggcagtcaacttctcaaggtcaagagcagctcctggaggcgcgaacggttttacaagttgcaagaagattgcaaaacaatttggcaagagagccggaaagtcatgagaactcccgagagccaactgttcagcatcgaggacatccaagaagtcaggatgggccataggaccgagggcttggaaaaattcgccagggacgtgcccgaagaccgatgctttagcatcgtgttcaaagatcagaggaacacgttggacttgatcgcccccagtccggctgatgcccagcattgggttctggggctccataagatcatccatcatagcggcagcatggaccagaggcagaaactccaacattggattcatagttgtcttaggaaagccgacaagaacaaggacaacaagatgagcttcaaggagttgcagaattttcttaaagagctgaatatccagtaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 65 - DNA, 11350 bp, synthetic construct
  misc_feature 1..11350: note = Myr (n-term; lyn pal-)
  source 1..11350: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgccatcaaatcaaagaggaaggataatctgaatgacgatgaatcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgtccggggtggtgcggaccctcagccgctgcctgctgccggccgaggccggctcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 66 - DNA, 5678 bp, synthetic construct
  source 1..5678: mol_type = other DNA
  misc_feature 1..5678: note = Single Pal, from GNA12
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgtccggggtggtgcggaccctcagccgctgcctgctgccggccgaggccggctcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 67 - DNA, 5690 bp, synthetic construct
  misc_feature 1..5690: note = Double Pal, from GNA13
  source 1..5690: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggcggacttcctgccgtcgcggtccgtgctgtccgtgtgcttccccggctgcctgctgacgagttcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 68 - DNA, 5690 bp, synthetic construct
  misc_feature 1..5690: note = Triple Pal from GNA15
  source 1..5690: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggcccgctcgctgacctggcgctgctgcccctggtgcctgacggaggatgagaaggccgccgcctcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 69 - DNA, 5672 bp, synthetic construct
  misc_feature 1..5672: note = Myr-pal (lyn), N-term
  source 1..5672: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggctgcatcaaatcaaagaggaaggataatctgaatgacgatgaatcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 70 - DNA, 11695 bp, synthetic construct
  misc_feature 1..11695: note = Farnesyl, c-term, from HRAS
  source 1..11695: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattcccagaactaccccatcgtgcagaagctgaaccctcctgatgagagtggccccggctgcatgagctgcaagtgtgtgctctcctgataaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggaaacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 71 - DNA, 6008 bp, synthetic construct
  source 1..6008: mol_type = other DNA
  misc_feature 1..6008: note = HIV MA (N-term)
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggaaacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 72 - protein, 489 aa, synthetic construct
  REGION 1..489: note = pMA2 N-term GAG-T2A-Cre (no promoter; no Met)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAGSGEGRGSLLTCGDVEENPGPMVPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 73 - protein, 624 aa, synthetic construct
  REGION 1..624: note = MA-MS2cp-CA gagpol (no Met)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 74 - protein, 252 aa, synthetic construct
  REGION 1..252: note = HIV MA (N-term)-MS2cp (no Met)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 75 - protein, 478 aa, synthetic construct
  REGION 1..478: note = HIV MA (N-term)-Cre (no Met)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 76 - protein, 21 aa, synthetic construct
  REGION 1..21: note = T2A
GSGEGRGSLLTCGDVEENPGP

SEQ ID NO: 77 - protein, 138 aa, synthetic construct
  REGION 1..138: note = gag-T2A (no Met)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAGSGEGRGSLLTCGDVEENPGP

SEQ ID NO: 78 - protein, 135 aa, synthetic construct
  REGION 1..135: note = HIV MA-cleavage site (no Met)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQ

SEQ ID NO: 79 - protein, 116 aa, synthetic construct
  REGION 1..116: note = MS2cp (no Met)
ASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 80 - protein, 245 aa, synthetic construct
  REGION 1..245: note = CD8 scFv
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 81 - protein, 247 aa, synthetic construct
  REGION 1..247: note = CD8 scFv
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 82 - protein, 240 aa, synthetic construct
  REGION 1..240: note = CD8 scFv
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 83 - protein, 241 aa, synthetic construct
  REGION 1..241: note = CD8 scFv
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSSGGGGSGGGGSGGGGSDIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 84 - protein, 125 aa, synthetic construct
  REGION 1..125: note = CD8 VHH
QVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

SEQ ID NO: 85 - protein, 5 aa, synthetic construct
  REGION 1..5: note = CDR-H1
SYAIS

SEQ ID NO: 86 - protein, 17 aa, synthetic construct
  REGION 1..17: note = CDR-H2
IIDPSDGNTNYAQNFQG

SEQ ID NO: 87 - protein, 13 aa, synthetic construct
  REGION 1..13: note = CDR-H3
ERAAAGYYYYMDV

SEQ ID NO: 88 - protein, 11 aa, synthetic construct
  REGION 1..11: note = CDR-L1
RASQSISSYLN

SEQ ID NO: 89 - protein, 7 aa, synthetic construct
  REGION 1..7: note = CDR-L2
AASSLQS

SEQ ID NO: 90 - protein, 9 aa, synthetic construct
  REGION 1..9: note = CDR-L3
QQSYSTPLT

SEQ ID NO: 91 - protein, 122 aa, synthetic construct
  REGION 1..122: note = VH
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 92 - protein, 108 aa, synthetic construct
  REGION 1..108: note = VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 93 - protein, 5 aa, synthetic construct
  REGION 1..5: note = CDR-H1
DYYIQ

SEQ ID NO: 94 - protein, 17 aa, synthetic construct
  REGION 1..17: note = CDR-H2
WINPNSGGTSYAQKFQG

SEQ ID NO: 95 - protein, 10 aa, synthetic construct
  REGION 1..10: note = CDR-H3
EGDYYYGMDA

SEQ ID NO: 96 - protein, 16 aa, synthetic construct
  REGION 1..16: note = CDR-L1
RSSQSLLHSNGYNYLD

SEQ ID NO: 97 - protein, 7 aa, synthetic construct
  REGION 1..7: note = CDR-L2
LGSNRAS

SEQ ID NO: 98 - protein, 9 aa, synthetic construct
  REGION 1..9: note = CDR-L3
MQGLQTPHT

SEQ ID NO: 99 - protein, 119 aa, synthetic construct
  REGION 1..119: note = VH
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 100 - protein, 113 aa, synthetic construct
  REGION 1..113: note = VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 101 - protein, 5 aa, synthetic construct
  REGION 1..5: note = CDR-H1
SYYMH

SEQ ID NO: 102 - protein, 17 aa, synthetic construct
  REGION 1..17: note = CDR-H2
GFDPEDGETIYAQKFQG

SEQ ID NO: 103 - protein, 8 aa, synthetic construct
  REGION 1..8: note = CDR-H3
DQGWGMDV

SEQ ID NO: 104 - protein, 9 aa, synthetic construct
  REGION 1..9: note = CDR-L3
QQTYSTPYT

SEQ ID NO: 105 - protein, 117 aa, synthetic construct
  REGION 1..117: note = VH
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 106 - protein, 108 aa, synthetic construct
  REGION 1..108: note = VL
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 107 - protein, 5 aa, synthetic construct
  REGION 1..5: note = CDR-H1
NHYMH

SEQ ID NO: 108 - protein, 17 aa, synthetic construct
  REGION 1..17: note = CDR-H2
WMNPNSGNTGYAQKFQG

SEQ ID NO: 109 - protein, 9 aa, synthetic construct
  REGION 1..9: note = CDR-H3
SESGSDLDY

SEQ ID NO: 110 - protein, 11 aa, synthetic construct
  REGION 1..11: note = CDR-L1
RASQTIGNYVN

SEQ ID NO: 111 - protein, 7 aa, synthetic construct
  REGION 1..7: note = CDR-L2
GASNLHT

SEQ ID NO: 112 - protein, 9 aa, synthetic construct
  REGION 1..9: note = CDR-L3
QQTYSAPLT

SEQ ID NO: 113 - protein, 118 aa, synthetic construct
  REGION 1..118: note = VH
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 114 - protein, 108 aa, synthetic construct
  REGION 1..108: note = VL
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 115 - protein, 5 aa, synthetic construct
  REGION 1..5: note = CDR-H1
GYVMG

SEQ ID NO: 116 - protein, 17 aa, synthetic construct
  REGION 1..17: note = CDR-H2
AISRGGLSTSYADSVKG

SEQ ID NO: 117 - protein, 16 aa, synthetic construct
  REGION 1..16: note = CDR-H3
DRSDLYEITAASNIDS

SEQ ID NO: 118 - protein, 5 aa, synthetic construct
  REGION 1..5: note = CD3 CDR-H1
RYTMH

SEQ ID NO: 119 - protein, 17 aa, synthetic construct
  REGION 1..17: note = CD3 CDR-H2
YINPSRGYTNYNQKFKD

SEQ ID NO: 120 - protein, 10 aa, synthetic construct
  REGION 1..10: note = CD3 CDR-H3
YYDDHYCLDY

SEQ ID NO: 121 - protein, 10 aa, synthetic construct
  REGION 1..10: note = CD3 CDR-L1
RASSSVSYMN

SEQ ID NO: 122 - protein, 7 aa, synthetic construct
  REGION 1..7: note = CD3 CDR-L2
DTSKVAS

SEQ ID NO: 123 - protein, 9 aa, synthetic construct
  REGION 1..9: note = CD3 CDR-L3
QQWSSNPLT

SEQ ID NO: 124 - protein, 121 aa, synthetic construct
  REGION 1..121: note = CD3 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 125 - protein, 106 aa, synthetic construct
  REGION 1..106: note = CD3 VL
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 126 - protein, 243 aa, synthetic construct
  REGION 1..243: note = CD3 scFv
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 127 - protein, 131 aa, synthetic construct
  REGION 1..131: note = HIV MA (no Met)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNY

SEQ ID NO: 128 - protein, 8 aa, synthetic construct
  REGION 1..8: note = cleavage site
SQNYPIVQ

SEQ ID NO: 129 - protein, 368 aa, synthetic construct
  REGION 1..368: note = CA
PIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 130 - protein, 372 aa, synthetic construct
  REGION 1..372: note = CA-cleavage site
SQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 131 - protein, 118 aa, synthetic construct
  REGION 1..118: note = N-term gag
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQA

SEQ ID NO: 132 - protein, 490 aa, synthetic construct
  REGION 1..490: note = pMA2 N-term GAG-T2A-Cre (no promoter)
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAGSGEGRGSLLTCGDVEENPGPMVPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 133 - protein, 625 aa, synthetic construct
  REGION 1..625: note = MA-MS2cp-CA gagpol
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 134 - protein, 253 aa, synthetic construct
  REGION 1..253: note = HIV MA (N-term)-MS2cp
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 135 - protein, 479 aa, synthetic construct
  REGION 1..479: note = HIV MA (N-term)-Cre
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 136 - protein, 139 aa, synthetic construct
  REGION 1..139: note = gag-T2A
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAGSGEGRGSLLTCGDVEENPGP

SEQ ID NO: 137 - protein, 136 aa, synthetic construct
  REGION 1..136: note = HIV MA-cleavage site
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQ

SEQ ID NO: 138 - protein, 117 aa, synthetic construct
  REGION 1..117: note = MS2cp
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 139 - protein, 132 aa, synthetic construct
  REGION 1..132: note = HIV MA
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNY

SEQ ID NO: 140 - protein, 4 aa, synthetic construct
  REGION 1..4: note = cleavage site
PIVQ

SEQ ID NO: 141 - DNA, 5880 bp, synthetic construct
  misc_feature 1..5880: note = N-term mutated MSD GAG-T2A-Cre
  source 1..5880: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgcagagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcaggtagtggcgagggcagaggaagtcttctaacatgcggtgacgtggaggagaatcccggccctatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 142 - DNA, 5544 bp, synthetic construct
  misc_feature 1..5544: note = GAG-T2A-EGFP
  source 1..5544: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcaggtagtggcgagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 143 - DNA, 5544 bp, synthetic construct; identical to SEQ ID NO: 142
  misc_feature 1..5544: note = N-term mutated MSD GAG-T2A-EGFP
  source 1..5544: mol_type = other DNA

SEQ ID NO: 144 - protein, 524 aa, synthetic construct; identical to SEQ ID NO: 16
  REGION 1..524: note = NiV F for VLP

SEQ ID NO: 145 - protein, 523 aa, synthetic construct
  REGION 1..523: note = NiV F for VLP without N term Methionine
VVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 146 - protein, 569 aa, synthetic construct; identical to SEQ ID NO: 42
  REGION 1..569: note = Niv G for VLP

SEQ ID NO: 147 - protein, 568 aa, synthetic construct
  REGION 1..568: note = Niv G for VLP without N term Methionine
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 148 - DNA, 5890 bp, synthetic construct
  misc_feature 1..5890: note = MS2cp-EGFP mRNA; HIV MA (N-term)
  source 1..5890: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatccaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 149 - DNA, 5698 bp, synthetic construct
  misc_feature 1..5698: note = MA-EGFP; HIV MA (N-term)
  source 1..5698: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggaaacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 150 - DNA, 5684 bp, synthetic construct
  misc_feature 1..5684: note = MA-tdMS2
  source 1..5684: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggaaacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgccatggccagcaacttcacccagttcgtgctggtggacaacggcggcaccggcgacgtgaccgtggcccccagcaacttcgccaacggcatcgccgagtggatcagcagcaacagcagaagccaggcctacaaggtgacatgcagcgtgagacagagcagcgcccagaacagaaagtacaccatcaaggtggaggtgcccaagggcgcctggagaagctacctgaacatggagctgaccatccccatcttcgccaccaacagcgactgcgagctgatcgtgaaggccatgcagggcctgctgaaggacggcaaccccatccccagcgccatcgccgccaacagcggcatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 151 - DNA, 6457 bp, synthetic construct
  misc_feature 1..6457: note = VSVG-MS2cp
  source 1..6457: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagatggccagcaattttacgcaattcgtgctcgtggacaacggcggcacgggcgacgtgaccgtggcccccagcaacttcgccaatggcatcgccgaatggatcagcagcaacagcaggagccaggcgtataaagttacgtgcagcgtcagacagagcagcgcccagaacaggaaatatacgatcaaggtcgaggttcccaagggagcttggaggagctatcttaatatggagctgaccatccccatcttcgcgacaaattcagactgcgagctcatcgtgaaggcaatgcagggcctcttgaaagatggcaaccccatcccaagcgcaatcgcggccaactcaggaatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 152 - DNA, 6811 bp, synthetic construct
  misc_feature 1..6811: note = VSVG-td-MS2cp
  source 1..6811: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgccatggccagcaacttcacccagttcgtgctggtggacaacggcggcaccggcgacgtgaccgtggcccccagcaacttcgccaacggcatcgccgagtggatcagcagcaacagcagaagccaggcctacaaggtgacctgcagcgtgagacagagcagcgcccagaacagaaagtacaccatcaaggtggaggtgcccaagggcgcctggagaagctacctgaacatggagctgaccatccccatcttcgccaccaacagcgactgcgagctgatcgtgaaggccatgcagggcctgctgaaggacggcaaccccatccccagcgccatcgccgccaacagcggcatctactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 153 - DNA, 5045 bp, synthetic construct
  misc_feature 1..5045: note = MA (N-term)- lamda N
  source 1..5045: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggaaacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagatggacgcccagaccaggaggagggaaaggagggcagagaagcaggcccaatggaaggccgccaactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 154 - DNA, 5045 bp, synthetic construct
  misc_feature 1..5045: note = MA (N-term)- mutLamdaN
  source 1..5045: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggaaacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagggaaacgcccggaccaggaggagggaaaggagggcagagaagcaggcccaatggaaggccgccaactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 155 - DNA, 6172 bp, synthetic construct
  misc_feature 1..6172: note = VSVG- LamdaN
  source 1..6172: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagatggacgcccagaccaggaggagggaaaggagggcagagaagcaggcccaatggaaggccgccaactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 156 - DNA, 6172 bp, synthetic construct
  misc_feature 1..6172: note = VSVG- mutLamdaN
  source 1..6172: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagggaaacgcccggaccaggaggagggaaaggagggcagagaagcaggcccaatggaaggccgccaactaaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 157 - protein, 628 aa, synthetic construct
  REGION 1..628: note = VSVG-MS2cp aa
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 158 - protein, 746 aa, synthetic construct
  REGION 1..746: note = VSVG-td-MS2cp
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 159 - protein, 158 aa, synthetic construct
  REGION 1..158: note = MA (N-term)- LamdaN
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMDAQTRRRERRAEKQAQWKAAN

SEQ ID NO: 160 - protein, 158 aa, synthetic construct
  REGION 1..158: note = MA (N-term)- mutLamdaN
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQGNARTRRRERRAEKQAQWKAAN

SEQ ID NO: 161 - protein, 533 aa, synthetic construct
  REGION 1..533: note = VSVG- LamdaN
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKMDAQTRRRERRAEKQAQWKAAN

SEQ ID NO: 162 - protein, 533 aa, synthetic construct
  REGION 1..533: note = VSVG- mutLamdaN
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKGNARTRRRERRAEKQAQWKAAN

SEQ ID NO: 163 - DNA, 5671 bp, synthetic construct
  misc_feature 1..5671: note = 1x MS2 with Cre
  source 1..5671: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatcctaaggtacctaattgcctagaaaacatgaggatcacccatgtctgcaggtcgactctagaaagtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 164 - DNA, 5718 bp, synthetic construct
  misc_feature 1..5718: note = 2x MS2 with Cre
  source 1..5718: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatcctaaggtacctaattgcctagaaaacatgaggatcacccatgtctgcaggtcgactctagaaaacatgaggatcacccatgtctgcagtattcccgggttcattagatccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 165 - DNA, 5928 bp, synthetic construct
  misc_feature 1..5928: note = 6x MS2 with Cre
  source 1..5928: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 166 - DNA, 6229 bp, synthetic construct
  misc_feature 1..6229: note = 12x MS2 with Cre
  source 1..6229: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 167 - DNA, 6884 bp, synthetic construct
  misc_feature 1..6884: note = 24x MS2 with Cre
  source 1..6884: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgagcatacattgtgcctatttcttgggtggacgatcacgccacccatgctctcacgaatttcaaaacacggacaaggacgagcaccaccagggctcgtcgttccacgtccaatacgattacttacctttcgggatcacgatcacggatcccgcagctacatcacttccactcaggacattcaagcatgcacgatcacggcatgctccacaagtctcaaccacagaaactaccaaatgggttcagcaccagcgaacccactcctacctcaaacctcttcccacaaaactggcaagcaggatcaccgcttgcccattccaacataccaaatcaaaaacaattactggtacagcatcagcgtaccagcccacatctctcactactatcaaaaaccaaaccgttcagcaacagcgaacggtacacacggaaaaatcaactggtttacaaatacgaaagacgatcacgctttcgtccagcgcaaactattacgaaaaacatccgacgggaagagcaacagccttcccgcggcggaaaacctcacaaaaacacgacaaacggatgcacgaacacggcatccgccgacaacccacaaacttacaaccaggcaaacggtgcaggatcaccgcaccgtacatcaaacacctcagatctcatgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 168 - DNA, 5659 bp, synthetic construct
  misc_feature 1..5659: note = 1x boxB with Cre
  source 1..5659: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaagtccaactactaaactggggattcctgggccctgaagaagggcccctcgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 169 - DNA, 5705 bp, synthetic construct
  misc_feature 1..5705: note = 2x boxB with Cre
  source 1..5705: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaagtccaactactaaactggggattcctgggccctgaagaagggcccctcgactaagtccaactactaaactgggccctgaagaagggcccatatagtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 170 - DNA, 5839 bp, synthetic construct
  misc_feature 1..5839: note = 5x boxB with Cre
  source 1..5839: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 171 - DNA, 6073 bp, synthetic construct
  misc_feature 1..6073: note = 10x boxB with Cre
  source 1..6073: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtaagcaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 172 - DNA, 6260 bp, synthetic construct
  source 1..6260: mol_type = other DNA
  misc_feature 1..6260: note = 20x boxB with Cre
tatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtaagcaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgaaaaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 173 - DNA, 6541 bp, synthetic construct
  misc_feature 1..6541: note = 20x boxB with Cre
  source 1..6541: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgaggatccaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtaagcaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgaaaaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagttcccaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 174 - DNA, 60 bp, synthetic construct
  misc_feature 1..60: note = 1x MS2 without Cre
  source 1..60: mol_type = other DNA
ggatcctaaggtacctaattgcctagaaaacatgaggatcacccatgtctgcaggtcgac

SEQ ID NO: 175 - DNA, 121 bp, synthetic construct
  misc_feature 1..121: note = 2x MS2 without Cre
  source 1..121: mol_type = other DNA
ggatcctaaggtacctaattgcctagaaaacatgaggatcacccatgtctgcaggtcgactctagaaaacatgaggatcacccatgtctgcagtattcccgggttcattagatccgtcgac

SEQ ID NO: 176 - DNA, 331 bp, synthetic construct
  misc_feature 1..331: note = 6x MS2 without Cre
  source 1..331: mol_type = other DNA
ggatccaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgac

SEQ ID NO: 177 - DNA, 638 bp, synthetic construct
  misc_feature 1..638: note = 12x MS2 without Cre
  source 1..638: mol_type = other DNA
ggatccaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgacctcgag

SEQ ID NO: 178 - DNA, 1286 bp, synthetic construct
  misc_feature 1..1286: note = 24x MS2 without Cre
  source 1..1286: mol_type = other DNA
ggatccaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgagcatacattgtgcctatttcttgggtggacgatcacgccacccatgctctcacgaatttcaaaacacggacaaggacgagcaccaccagggctcgtcgttccacgtccaatacgattacttacctttcgggatcacgatcacggatcccgcagctacatcacttccactcaggacattcaagcatgcacgatcacggcatgctccacaagtctcaaccacagaaactaccaaatgggttcagcaccagcgaacccactcctacctcaaacctcttcccacaaaactggcaagcaggatcaccgcttgcccattccaacataccaaatcaaaaacaattactggtacagcatcagcgtaccagcccacatctctcactactatcaaaaaccaaaccgttcagcaacagcgaacggtacacacggaaaaatcaactggtttacaaatacgaaagacgatcacgctttcgtccagcgcaaactattacgaaaaacatccgacgggaagagcaacagccttcccgcggcggaaaacctcacaaaaacacgacaaacggatgcacgaacacggcatccgccgacaacccacaaacttacaaccaggcaaacggtgcaggatcaccgcaccgtacatcaaacacctcagatctcatgtcga

SEQ ID NO: 179 - DNA, 56 bp, synthetic construct
  misc_feature 1..56: note = 1x boxB without Cre
  source 1..56: mol_type = other DNA
aagtccaactactaaactggggattcctgggccctgaagaagggcccctcgtcgac

SEQ ID NO: 180 - DNA, 102 bp, synthetic construct
  misc_feature 1..102: note = 2x boxB without Cre
  source 1..102: mol_type = other DNA
aagtccaactactaaactggggattcctgggccctgaagaagggcccctcgactaagtccaactactaaactgggccctgaagaagggcccatatagtcgac

SEQ ID NO: 181 - DNA, 236 bp, synthetic construct
  misc_feature 1..236: note = 5x boxB without Cre
  source 1..236: mol_type = other DNA
aagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgtcgac

SEQ ID NO: 182 - DNA, 470 bp, synthetic construct
  misc_feature 1..470: note = 10x boxB without Cre
  source 1..470: mol_type = other DNA
aagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtaagcaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgtcgac

SEQ ID NO: 183 - DNA, 704 bp, synthetic construct
  misc_feature 1..704: note = 15x boxB without Cre
  source 1..704: mol_type = other DNA
aagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtaagcaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgaaaaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgtcgac

SEQ ID NO: 184 - DNA, 938 bp, synthetic construct
  misc_feature 1..938: note = 20x boxB without Cre
  source 1..938: mol_type = other DNA
aagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtaagcaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgaaaaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagttcccaagtccaactactaaactggggattcctgggccctgaagaagggcccctcgactaagtccaactactaaactgggccctgaagaagggcccatatagggccctgaagaagggccctatcgaggatattatctcgactaagtccaactactaaactgggccctgaagaagggcccatatagggccctgaagaagggccctatcgaggatattatcaatcttcctgctcagtgtcgac

SEQ ID NO: 185 - skipped

SEQ ID NO: 186 - DNA, 18 bp, synthetic construct
  misc_feature 1..18: note = BoxB stem loop
  source 1..18: mol_type = other DNA
gggccctgaagaagggcc

SEQ ID NO: 187 - protein, 22 aa, synthetic construct
  REGION 1..22: note = Phage lambda N protein
MDAQTRRRERRAEKQAQWKAAN

SEQ ID NO: 188 - protein, 22 aa, synthetic construct
  REGION 1..22: note = Mutated phage lambda N protein
MDAQTRRRERRAEKQAQWKAAN

SEQ ID NO: 189 - protein, 512 aa, synthetic construct
  REGION 1..512: note = VSV-G
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKM

SEQ ID NO: 190 - protein, 371 aa, synthetic construct
  REGION 1..371: note = MA-td-MS2cp (aa)
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 191 - protein, 370 aa, synthetic construct
  REGION 1..370: note = MA-td-MS2cp no methionine (aa)
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 192 - protein, 627 aa, synthetic construct
  REGION 1..627: note = VSVG-MS2cp no methionine aa
KCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 193 - protein, 745 aa, synthetic construct
  REGION 1..745: note = VSVG-td-MS2cp no methionine
KCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 194 - protein, 157 aa, synthetic construct
  REGION 1..157: note = MA- LamdaN no methionine
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQMDAQTRRRERRAEKQAQWKAAN

SEQ ID NO: 195 - protein, 157 aa, synthetic construct
  REGION 1..157: note = MA-mutLamdaN no methionine
GARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQGNARTRRRERRAEKQAQWKAAN

SEQ ID NO: 196 - protein, 532 aa, synthetic construct
  REGION 1..532: note = VSVG- LamdaN no methionine
KCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKMDAQTRRRERRAEKQAQWKAAN

SEQ ID NO: 197 - protein, 532 aa, synthetic construct
  REGION 1..532: note = VSVG- mutLamdaN no methionine
KCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKGNARTRRRERRAEKQAQWKAAN

SEQ ID NO: 198 - protein, 235 aa, synthetic construct
  REGION 1..235: note = tdMS2
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 199 - protein, 511 aa, synthetic construct
  REGION 1..511: note = VSV-G
KCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKM

SEQ ID NO: 200 - DNA, 5880 bp, synthetic construct; identical to SEQ ID NO: 51
  misc_feature 1..5880: note = GAG-T2A-EGFP
  source 1..5880: mol_type = other DNA

SEQ ID NO: 201 - protein, 351 aa, synthetic construct
  REGION 1..351: note = Nuclear localization signal Cre
MVPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 202 - protein, 343 aa, synthetic construct
  REGION 1..343: note = Cre
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 203 - protein, 239 aa, synthetic construct
  REGION 1..239: note = EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 204 - DNA, 4827 bp, synthetic construct
  misc_feature 1..4827: note = pMA2 N-term mutated MSD GAG-T2A
  source 1..4827: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgcagagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcaggtagtggcgagggcagaggaagtcttctaacatgcggtgacgtggaggagaatcccggcccttgaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 205 - DNA, 4827 bp, synthetic construct
  misc_feature 1..4827: note = pMA2 N-term GAG-T2A
  source 1..4827: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcaggtagtggcgagggcagaggaagtcttctaacatgcggtgacgtggaggagaatcccggcccttgaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 206 - DNA, 5171 bp, synthetic construct
  source 1..5171: mol_type = other DNA
  misc_feature 1..5171: note = MS2cp mRNA; HIV MA (N-term)
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaggatccaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 207 - DNA, 4979 bp, synthetic construct
  source 1..4979: mol_type = other DNA
  misc_feature 1..4979: note = MA; HIV MA (N-term)
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccaccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggaaacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 208 - DNA, 19 bp, synthetic construct
  source 1..19: mol_type = other DNA
  misc_feature 1..19: note = MS2 stem loop configuration
nrndsassancasssnnyn

(19) United States
(12) Patent Application Publication
(10) Pub. No.: US 2026/0055146 A1
(43) Pub. Date: Feb. 26, 2026

(54) DELIVERY OF HETEROLOGOUS PROTEINS

(71) Applicant: Sana Biotechnology, Inc., Seattle, WA (US)

(72) Inventors: Michael J. VOLLES, Seattle, WA (US); Jillian DIERX, Cambridge, MA (US); Philip CALAFATI, Cambridge, MA (US)

(73) Assignee: Sana Biotechnology, Inc., Seattle, WA (US)

(21) Appl. No.: 19/105,428

(22) PCT Filed: Aug. 23, 2023
(86) PCT No.: PCT/US2023/072772

§ 371 Date: Feb. 21, 2025

Related U.S. Application Data
(60) Provisional application No. 63/400,662, filed on Aug. 24, 2022.

(51) Int. Cl. C07K 14/16; C12N 5/071; C12N 15/62; C12N 15/867; C12N 15/88

(52) U.S. Cl. CPC C07K 14/162; C07K 14/161; C07K 14/163; C12N 5/0602; C12N 15/62; C12N 15/867; C12N 15/88; C07K 2319/03; C12N 2740/16023; C12N 2740/16043; C12N 2740/16045; C12N 2840/44

(57) ABSTRACT

Provided herein are lipid particles and compositions thereof for delivery of heterologous proteins, including genome-modifying agents, to cells.

### CROSS-REFERENCE TO RELATED APPLICATIONS

This application claims priority from U.S. provisional application No. 63/400,662, filed Aug. 24, 2022, entitled “DELIVERY OF HETEROLOGOUS PROTEINS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 186152007140SeqList created Aug. 24, 2023, which is 632,448 kilobytes in size. The information in the electronic format of the Sequence Listing is incorporated by reference in its entirety.

### FIELD

The present disclosure relates to lipid particles and compositions thereof for use in delivering heterologous proteins, including genome-modifying agents, to cells.

### BACKGROUND

Lipid particles, including viral-derived particles, are used for delivery of heterologous proteins, such as genome-modifying agents, to cells. However, delivery of genome-modifying agents, including delivery of multiple exogenous genome-modifying agents to certain target cells can be challenging. Improved lipid particles, including viral-derived particles, for use in methods of delivering genome-modifying agents to targeted cells are needed. The provided disclosure addresses this need.

### SUMMARY

Provided herein is a lipid particle having a lipid bilayer enclosing a lumen and a ribonucleic acid (RNA), wherein the RNA includes, from 5′ to 3′: a R element of a 5′ long terminal repeat (5′ LTR); a U5 element of a 5′ LTR; a RNA sequence encoding a gag protein or portion thereof containing at least a gag start codon; a RNA sequence encoding a heterologous protein that is operably linked to the RNA sequence encoding a gag protein or portion thereof; and a poly-A tail, wherein each of the R element of the 5′ LTR, the U5 element of the 5′ LTR, and the RNA sequence encoding a gag protein or portion thereof is retroviral. In some embodiments, the RNA contains a retroviral packaging sequence that is 3′ to the 5′ LTR.

Also provided herein is a lipid particle having a lipid bilayer enclosing a lumen and a ribonucleic acid (RNA), wherein the RNA includes, from 5′ to 3′: a R element of a 5′ long terminal repeat (5′ LTR); a U5 element of a 5′ LTR; a gag 5′ untranslated region (UTR) or portion thereof containing at least three nucleotides; a RNA sequence encoding a heterologous protein that is operably linked to the gag 5′ UTR or a portion thereof; and a poly-A tail, wherein each of the R element of the 5′ LTR, the U5 element of the 5′ LTR, and the gag 5′ UTR or portion thereof is retroviral. In some embodiments, the RNA contains a retroviral packaging sequence that is 3′ to the 5′ LTR.

Also provided herein is a lipid particle having a lipid bilayer enclosing a lumen and a ribonucleic acid (RNA), wherein the RNA includes, from 5′ to 3′: a R element of a 5′ long terminal repeat (5′ LTR); a U5 element of a 5′ LTR; a retroviral packaging sequence; a gag start codon; a RNA sequence encoding a heterologous protein; and a poly-A tail, wherein each of the R element of the 5′ LTR, the U5 element of the 5′ LTR, and the gag start codon is retroviral.

In some embodiments, the lipid particle further includes a U3 element of a 5′ LTR. In some embodiments, the RNA includes a polyadenylation site. In some embodiments, the RNA includes a 3′ long terminal repeat (3′ LTR), and the polyadenylation site is located within a 3′ LTR. In some embodiments, the RNA includes a mutated primer binding site (PBS).

In some embodiments, wherein the retroviral packaging sequence is selected from the group comprising HIV psi, MLV psi, SNV E, or a portion of any thereof. In some embodiments, the retroviral packaging sequence comprises stem-loop 1 (SL1) of HIV psi. In some embodiments, the retroviral packaging sequence comprises stem-loop 2 (SL2) of HIV psi. In some embodiments, the retroviral packaging sequence comprises stem-loop 3 (SL3) of HIV psi.

In some embodiments, the retroviral packaging sequence comprises stem-loop 4 (SL4) of HIV psi.

In some of any of the provided embodiments, the retroviral packaging sequence is HIV psi. In some of any of the provided embodiments, the retroviral packaging sequence comprises a mutation in a major splice donor site. In some of any of the provided embodiments, the major splice donor site is a major splice donor site contained in SL2 of HIV psi. In some of any of the provided embodiments, the mutation is a mutation that inhibits splicing at the major splice donor site. In some of any of the provided embodiments, the mutation in the major splice donor site comprises a mutation that prevents splicing at the major splice donor site.

In some embodiments, the RNA includes a retroviral sequence having at least about 80% sequence identity to the sequence of a retroviral genome that is about 20, 30, 40, 50, 60, 70, 80, 90, 100, 120, 140, 160, 180, 200, 220, 240, 260, 280, 300, 320, 340, 360, 380, or 400 nucleotides in length and contains the gag start codon. In some embodiments, the RNA includes a retroviral sequence having at least about 80% sequence identity to the sequence of a retroviral genome that is about 20, 30, 40, 50, 60, 70, 80, 90, 100, 120, 140, 160, 180, 200, 220, 240, 260, 280, 300, 320, 340, 360, 380, or 400 nucleotides in length and contains a gag start codon. In some embodiments, the retroviral sequence contains between about 20-400, between about 40 and about 350, between about 60 and about 300, between about 80 and about 250, or between about 100 and about 200 nucleotides 5′ to the gag start codon. In some embodiments, the retroviral sequence contains between about 20 and about 400, between about 40 and about 350, between about 60 and about 300, between about 80 and about 250, or between about 100 and about 200 nucleotides 3′ to the gag start codon.

In some embodiments, the lumen contains a capsid containing a retroviral capsid protein enclosing the RNA. In some embodiments, the retroviral capsid protein and the retroviral packaging sequence are capable of associating with each other. In some embodiments, the retroviral capsid protein and the retroviral packaging sequence are from the same retroviral species.

In some embodiments, the lipid particle contains a retroviral matrix protein.

In some embodiments, the lipid particle further contains a RNA sequence encoding a viral structural protein or a portion thereof, which is located between the gag start codon and the RNA sequence encoding a heterologous protein. In some embodiments, the RNA does not contain nucleotides between the gag start codon and the RNA sequence encoding a heterologous protein.

Also provided herein is a lipid particle having a lipid bilayer enclosing a lumen and a ribonucleic acid (RNA), wherein the RNA includes, from 5′ to 3′: a R element of a 5′ long terminal repeat (5′ LTR); a U5 element of a 5′ LTR; RNA sequence encoding a viral structural protein or a portion thereof; a RNA sequence encoding a heterologous protein; and a poly-A tail, wherein each of the R element of the 5′ LTR and the U5 element of the 5′ LTR is retroviral.

In some embodiments, the viral structural protein or a portion thereof is a retroviral structural protein or a portion thereof. In some embodiments, the RNA contains a bicistronic element located between the RNA sequence encoding the viral structural protein or a portion thereof and the RNA sequence encoding the heterologous protein. In some embodiments, the bicistronic element is an internal ribosome entry site (IRES) element or a sequence encoding a 2A self-cleaving peptide. In some embodiments, the bicistronic element is a sequence encoding a 2A self-cleaving peptide, and the 2A self-cleaving peptide is T2A. In some embodiments, T2A contains the sequence set forth in SEQ ID NO:76. In some embodiments. T2A is the sequence set forth in SEQ ID NO:76. In some embodiments, the RNA encodes, from a 5′ to 3′ direction: the viral structural protein or portion thereof, T2A, and the heterologous protein.

In some embodiments, the viral structural protein is a retroviral gag. In some embodiments, the RNA sequence encoding the viral structural protein or a portion thereof encodes an N-terminal portion of a retroviral gag. In some embodiments, the RNA sequence encoding the viral structural protein or a portion thereof contains the sequence set forth in SEQ ID NO:52. In some embodiments, the RNA sequence encoding the viral structural protein or a portion thereof is the sequence set forth in SEQ ID NO: 52. In some embodiments, the RNA encodes the sequence set forth in SEQ ID NO:77 and the heterologous protein.

In some embodiments, the RNA is present as a first genomic viral RNA and the lipid particle further contains a second genomic viral RNA. In some embodiments, the first genomic viral RNA and the second viral genomic RNA genome are identical. In some embodiments, the first genomic viral RNA and the second viral genomic RNA genome are different.

In some embodiments, the heterologous protein is a first heterologous protein and the lipid particle further contains one or more additional heterologous protein(s), wherein the lipid particle contains: (a) a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}; or (b) a fusion protein comprising a viral matrix (MA) protein and the one or more additional heterologous protein(s). In some embodiments, the heterologous protein is a first heterologous protein and the lipid particle further contains one or more additional heterologous protein(s), wherein the lipid particle contains a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) contains a MS2_{cp}-binding loop for binding to MS2_{cp}. In some embodiments, the heterologous protein is a first heterologous protein and the lipid particle further contains one or more additional heterologous protein(s), wherein the lipid particle contains a fusion protein containing a viral matrix (MA) protein and the one or more additional heterologous protein(s). In some embodiments, the heterologous protein is a first heterologous protein and the lipid particle further contains one or more additional heterologous protein(s), wherein the lipid particle contains: (a) a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) contains a MS2_{cp}-binding loop for binding to MS2_{cp}; and (b) a fusion protein containing a viral matrix (MA) protein and the one or more additional heterologous protein(s).

In some embodiments, the viral MA protein in (a) or (b) is derived from human immunodeficiency virus (HIV). In some embodiments, the viral MA protein in (a) is derived from human immunodeficiency virus (HIV). In some embodiments, the viral MA protein in (b) is derived from human immunodeficiency virus (HIV). In some embodiments, the viral MA protein in (a) and (b) is derived from human immunodeficiency virus (HIV).

In some embodiments, the viral MA protein in (a) or (b) contains the sequence set forth in SEQ ID NO:78. In some embodiments, the viral MA protein in (a) contains the sequence set forth in SEQ ID NO: 78. In some embodiments, the viral MA protein in (b) contains the sequence set forth in SEQ ID NO: 78. In some embodiments, the viral MA protein in (a) and (b) contain the sequence set forth in SEQ ID NO: 78.

In some embodiments, MS2_{cp} in (b) contains the sequence set forth in SEQ ID NO:79. In some embodiments, the fusion protein of (a) contains the sequence set forth in SEQ ID NO:74.

In some of any of the provided embodiments, the fusion protein of (a) comprises: the amino acid sequence of SEQ ID NOs: 134 or 190; or the amino acid sequence of SEQ ID NOs: 74 or 191; or the amino acid sequence encoded by the nucleic acid sequence set forth in SEQ ID NO: 62 or 150.

In some embodiments, the RNA contains a 5′ cap. In some embodiments, the RNA is a self-inactivating lentiviral vector genome.

Also provided herein is a lipid particle having a lipid bilayer enclosing a lumen; a fusion protein containing a viral matrix (MA) protein and a MS2 coat protein (MS2_{cp}); and an RNA sequence encoding a heterologous protein. In some embodiments, the RNA sequence encoding a heterologous protein contains a MS2_{cp}-binding loop.

In some of any of the provided embodiments, the MS2_{cp}-binding loop comprises the RNA sequence transcribed from the nucleic acid set forth in SEQ ID NO: 185 or SEQ ID NO: 174. In some of any of the provided embodiments, the MS2_{cp}-binding loop comprises the RNA sequence set forth in SEQ ID NO: 208. In some of any of the provided embodiments, the RNA sequence encoding a heterologous protein comprises a plurality of MS2_{cp}-binding loops. In some of any of the provided embodiments, the plurality of MS2_{cp}-binding loops comprises at or a at least 2, 5, 6, 10, 12, 15, 20, or 24 MS2_{cp}-binding loops. In some of any of the provided embodiments, the RNA sequence encoding a heterologous protein comprises a plurality of MS2_{cp}-binding loops comprising between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops.

In some of any of the provided embodiments, the plurality of MS2_{cp}-binding loops comprises the RNA sequence transcribed from a nucleic acid sequence selected from the group consisting of SEQ ID NOs: 175-178.

Also provided herein is a a lipid bilayer enclosing a lumen; a viral matrix (MA) protein; an RNA-binding protein; and an RNA sequence encoding a heterologous protein, wherein the RNA-binding protein is incorporated into the lipid particle as a fusion protein with the viral MA protein, and wherein the RNA sequence encoding the heterologous protein comprises a binding site for binding to the RNA-binding protein.

Also provided herein is a lipid particle having a lipid bilayer enclosing a lumen; a viral matrix (MA) protein; a MS2 coat protein (MS2_{cp}); and an RNA sequence encoding a heterologous protein, wherein the MS2_{cp} is incorporated into the lipid particle as a fusion protein with the viral MA protein, and wherein the RNA sequence encoding the heterologous protein contains a MS2_{cp}-binding loop for binding to MS2_{cp}.

In some of any of the provided embodiments, the RNA sequence encoding a heterologous protein comprises at or at least 2, 5, 6, 10, 12, 15, 20, or 24 binding sites for binding to the RNA-binding protein. In some of any of the provided embodiments, the RNA sequence encoding a heterologous protein comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20, binding sites for binding to the RNA-binding protein.

In some of any of the provided embodiments, the RNA-binding protein is MS2 coat protein (MS2_{cp}). In some of any of the provided embodiments, MS2_{cp} comprises the sequence set forth in SEQ ID NO: 79. In some of any of the provided embodiments, the RNA-binding protein is MS2_{cp} and the binding site is an MS2_{cp}-binding loop for binding to the MS2_{cp}. In some of any of the provided embodiments, the MS2_{cp}-binding loop comprises the RNA sequence transcribed from the nucleic acid set forth in SEQ ID NO: 185 or SEQ ID NO: 174. In some of any of the provided embodiments, the MS2_{cp}-binding loop comprises the RNA sequence set forth in SEQ ID NO: 208. In some of any of the provided embodiments, the RNA sequence encoding a heterologous protein comprises a plurality of MS2_{cp}-binding loops. In some of any of the provided embodiments, the plurality of MS2_{cp}-binding loops comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops.

In some of any of the provided embodiments, the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops. In some of any of the provided embodiments, the plurality of MS2_{cp}-binding loops comprises the RNA sequence transcribed from a nucleic acid sequence selected from the group consisting of SEQ ID NOs: 175-178. In some of any of the provided embodiments, the RNA-binding protein is lambda N protein (λN) or a functional variant thereof. In some of any of the provided embodiments, the λN or a functional variant thereof comprises the amino acid sequence of SEQ ID NO: 187 or an amino acid sequence having at least 90% or 95% sequence identity to the amino acid sequence of SEQ ID NO: 187. In some of any of the provided embodiments, the λN or a functional variant thereof comprises the amino acid sequence of SEQ ID NO: 187.

In some of any of the provided embodiments, the λN or a functional variant thereof comprises the amino acid sequence of SEQ ID NO: 188 or an amino acid sequence having at least 90% or 95% sequence identity to the amino acid sequence of SEQ ID NO: 188. In some of any of the provided embodiments, the λN or a functional variant thereof comprises the amino acid sequence of SEQ ID NO: 188. In some of any of the provided embodiments, the RNA-binding protein is λN or a functional variant thereof and the binding site is a boxB binding site for binding to the λN or a functional variant thereof. In some of any of the provided embodiments, the boxB binding site comprises the RNA sequence transcribed from the nucleic acid set forth in SEQ ID NO: 186.

In some of any of the provided embodiments, the RNA sequence encoding a heterologous protein comprises a plurality of boxB binding sites. In some of any of the provided embodiments, the plurality of boxB binding sites comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 boxB binding sites. In some of any of the provided embodiments, the plurality of boxB binding sites comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 boxB binding sites. In some of any of the provided embodiments, the plurality of boxB binding sites comprises the RNA sequence transcribed from a nucleic acid sequence selected from the group consisting of SEQ ID NOS: 179-184.

Provided herein is a lipid particle comprising a lipid bilayer enclosing a lumen; a viral matrix (MA) protein; a MS2 coat protein (MS2_{cp}); and an RNA sequence encoding a heterologous protein, wherein the MS2_{cp} is incorporated into the lipid particle as a fusion protein with the viral MA protein, and wherein the RNA sequence encoding the heterologous protein comprises a MS2_{cp}-binding loop for binding to MS2_{cp}.

In some embodiments, the viral MA protein is attached to a portion of the lipid bilayer that is in contact with the lumen. In some embodiments, the viral MA protein reversibly binds to the lipid bilayer. In some embodiments, the fusion protein contains, from a 5′ to 3′ direction: the viral MA protein and MS2_{cp}. In some embodiments, the RNA sequence encoding a heterologous protein contains a MS2_{cp}-binding loop. In some embodiments, the RNA sequence encoding a heterologous protein contains 12 MS2_{cp}-binding loops. In some embodiments, the RNA sequence encoding a heterologous protein contains 24 MS2_{cp}-binding loops.

In some embodiments, the viral MA protein is derived from human immunodeficiency virus (HIV). In some embodiments, the viral MA protein contains the sequence set forth in SEQ ID NO:78. In some embodiments, MS2_{cp} contains the sequence set forth in SEQ ID NO:79. In some embodiments, the fusion protein contains the sequence set forth in SEQ ID NO:74.

In some embodiments, the lipid particle contains a transfer plasmid encoding a guide RNA (gRNA). In some embodiments, the gRNA is a single guide RNA (sgRNA). In some embodiments, the gRNA is under the control of a U6 promoter.

In some embodiments, the heterologous protein is a first heterologous protein and the lipid particle further contains one or more additional heterologous protein(s), wherein the lipid particle includes: (a) a RNA containing a RNA sequence encoding the one or more additional heterologous protein(s) and a RNA sequence encoding a viral structural protein or a portion thereof; or (b) a fusion protein containing a viral matrix (MA) protein and the one or more additional heterologous protein(s). In some embodiments, the heterologous protein is a first heterologous protein and the lipid particle further contains one or more additional heterologous protein(s), wherein the lipid particle includes a RNA containing a RNA sequence encoding the one or more additional heterologous protein(s) and a RNA sequence encoding a viral structural protein or a portion thereof. In some embodiments, the heterologous protein is a first heterologous protein and the lipid particle further contains one or more additional heterologous protein(s), wherein the lipid particle includes a fusion protein containing a viral matrix (MA) protein and the one or more additional heterologous protein(s). In some embodiments, the heterologous protein is a first heterologous protein and the lipid particle further contains one or more additional heterologous protein(s), wherein the lipid particle includes: (a) a RNA containing a RNA sequence encoding the one or more additional heterologous protein(s) and a RNA sequence encoding a viral structural protein or a portion thereof; and (b) a fusion protein containing a viral matrix (MA) protein and the one or more additional heterologous protein(s).

In some embodiments, the viral structural protein is gag. In some embodiments, the RNA sequence encoding the viral structural protein or a portion thereof in (a) encodes an N-terminal portion of gag. In some embodiments, the RNA sequence encoding the viral structural protein or a portion thereof in (a) contains the sequence set forth in SEQ ID NO:52. In some embodiments, the viral MA protein in (b) is derived from human immunodeficiency virus (HIV). In some embodiments, the viral MA protein in (b) contains the sequence set forth in SEQ ID NO:78.

Also provided herein is a lipid particle having a lipid bilayer enclosing a lumen a fusion protein containing a viral matrix (MA) protein and a heterologous protein.

Also provided herein is a lipid particle having a lipid bilayer enclosing a lumen; a viral matrix (MA) protein; and a heterologous protein, wherein the heterologous protein is incorporated into the lipid particle as a fusion protein with the viral MA protein.

In some embodiments, the viral MA protein is attached to a portion of the lipid bilayer that is in contact with the lumen. In some embodiments, the viral MA protein reversibly binds to the lipid bilayer. In some embodiments, the fusion protein contains, from a 5′ to 3′ direction: the viral MA protein and the heterologous protein. In some embodiments, the viral MA protein is derived from human immunodeficiency virus (HIV). In some embodiments, the viral MA protein contains the sequence set forth in SEQ ID NO:78.

In some embodiments, the lipid particle contains a transfer plasmid encoding a guide RNA (gRNA). In some embodiments, the gRNA is a single guide RNA (sgRNA). In some embodiments, the gRNA is under the control of a U6 promoter.

In some embodiments, the heterologous protein is a first heterologous protein and the lipid particle further contains one or more additional heterologous protein(s), wherein the lipid particle includes: (a) a RNA containing a RNA sequence encoding the one or more additional heterologous protein(s) and a RNA sequence encoding a viral structural protein or a portion thereof; or (b) a viral matrix (MA) protein, a MS2 coat protein (MS2_{cp}), and a RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) contains a MS2_{cp}-binding loop for binding to MS2_{cp}. In some embodiments, the heterologous protein is a first heterologous protein and the lipid particle further contains one or more additional heterologous protein(s), wherein the lipid particle includes a RNA containing a RNA sequence encoding the one or more additional heterologous protein(s) and a RNA sequence encoding a viral structural protein or a portion thereof. In some embodiments, the heterologous protein is a first heterologous protein and the lipid particle further contains one or more additional heterologous protein(s), wherein the lipid particle includes a viral matrix (MA) protein, a MS2 coat protein (MS2_{cp}), and a RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) contains a MS2_{cp}-binding loop for binding to MS2_{cp}. In some embodiments, the heterologous protein is a first heterologous protein and the lipid particle further contains one or more additional heterologous protein(s), wherein the lipid particle includes: (a) a RNA containing a RNA sequence encoding the one or more additional heterologous protein(s) and a RNA sequence encoding a viral structural protein or a portion thereof; and (b) a viral matrix (MA) protein, a MS2 coat protein (MS2_{cp}), and a RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) contains a MS2_{cp}-binding loop for binding to MS2_{cp}.

In some embodiments, the viral structural protein in (a) is gag. In some embodiments, the RNA sequence encoding the viral structural protein or a portion thereof in (a) encodes an N-terminal portion of gag. In some embodiments, the RNA sequence encoding the viral structural protein or a portion thereof in (a) contains the sequence set forth in SEQ ID NO:52. In some embodiments, the viral MA protein in (b) is derived from HIV. In some embodiments, the viral MA protein in (b) contains the sequence set forth in SEQ ID NO:78. In some embodiments, MS2_{cp} in (b) contains the sequence set forth in SEQ ID NO:79. In some embodiments, the fusion protein of (b) contains the sequence set forth in SEQ ID NO:74. In some of any of the provided embodiments, the fusion protein of (b) comprises the sequence set forth in SEQ ID NO: 191.

Provided herein is a lipid particle comprising a lipid bilayer enclosing a lumen; a fusion protein comprising a viral envelope glycoprotein and an RNA-binding protein; and an RNA sequence encoding a heterologous protein, wherein the RNA sequence encoding the heterologous protein comprises a binding site for binding to the RNA-binding protein.

In some of any of the provided embodiments, the fusion protein comprises, from an N-terminus to C-terminus direction: the viral envelope glycoprotein and the RNA binding protein. In some of any of the provided embodiments, the RNA-binding protein is fused to the C-terminus of the viral envelope glycoprotein.

Provided herein is a lipid particle comprising a lipid bilayer enclosing a lumen; a fusion protein comprising a VSV-G protein or a functional variant thereof and an RNA-binding protein; and an RNA sequence encoding a heterologous protein, wherein the RNA sequence encoding the heterologous protein comprises a binding site for binding to the RNA-binding protein.

In some of any of the provided embodiments, the VSV-G or a functional variant thereof comprises the amino acid sequence of SEQ ID NO: 199, or an amino acid sequence having at least 90%, 91%, 92%, 93%, 94%, 95%, 96%, 97%, 98%, or 99% sequence identity to the amino acid sequence of SEQ ID NO: 199. In some of any of the provided embodiments, the VSV-G or a functional variant thereof comprises the amino acid sequence of SEQ ID NO: 199. In some of any of the provided embodiments, the RNA sequence encoding a heterologous protein comprises at or at least 2, 5, 6, 10, 12, 15, 20, or 24 binding sites for binding to the RNA-binding protein. In some of any of the provided embodiments, the RNA sequence encoding a heterologous protein comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20, binding sites for binding to the RNA-binding protein.

In some of any of the provided embodiments, the lipid particle is pseudotyped with the viral envelope glycoprotein. In some of any of the provided embodiments, the fusion protein comprises, from an N-terminus to C-terminus direction: the VSV-G protein or a functional variant thereof and the RNA binding protein. In some of any of the provided embodiments, the RNA-binding protein is fused to the C-terminus of the VSV-G protein or a functional variant thereof. In some of any of the provided embodiments, the RNA-binding protein is MS2 coat protein (MS2_{cp}). In some of any of the provided embodiments, MS2_{cp} comprises the sequence set forth in SEQ ID NO: 79. In some of any of the provided embodiments, the MS2_{cp} is a homodimer. In some of any of the provided embodiments, the MS2_{cp} is a tandem dimer. In some of any of the provided embodiments, the binding site is an MS2_{cp}-binding loop for binding to the MS2_{cp}. In some of any of the provided embodiments, the RNA sequence encoding a heterologous protein comprises a plurality of MS2_{cp}-binding loops for binding to the MS2_{cp}.

In some of any of the provided embodiments, the plurality of MS2_{cp}-binding loops comprises at or at least 2, 4, 6, 8, 10, 12, 14, 16, 18, 20, 22, or 24 MS2_{cp}-binding loops. In some of any of the provided embodiments, the plurality of MS2_{cp}-binding loops comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops. In some of any of the provided embodiments, the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops. In some of any of the provided embodiments, the RNA-binding protein is lambda N protein (λN) or a functional variant thereof. In some of any of the provided embodiments, the λN or a functional variant thereof comprises the amino acid sequence of SEQ ID NO: 187 or an amino acid sequence having at least 90% or 95% sequence identity to the amino acid sequence of SEQ ID NO: 187. In some of any of the provided embodiments, the λN or a functional variant thereof comprises the amino acid sequence of SEQ ID NO: 187. In some of any of the provided embodiments, the λN or a functional variant thereof comprises the amino acid sequence of SEQ ID NO: 188 or an amino acid sequence having at least 90% or 95% sequence identity to the amino acid sequence of SEQ ID NO: 188. In some of any of the provided embodiments, the λN or a functional variant thereof comprises the amino acid sequence of SEQ ID NO: 188. In some of any of the provided embodiments, the RNA-binding protein is λN or a functional variant thereof and the binding site is a boxB binding site for binding to the λN or a functional variant thereof. In some of any of the provided embodiments, the RNA sequence encoding a heterologous protein comprises a plurality of boxB binding sites.

In some of any of the provided embodiments, the plurality of boxB binding sites comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 boxB binding sites. In some of any of the provided embodiments, the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops. In some of any of the provided embodiments, the plurality of boxB binding sites comprises the RNA sequence transcribed from a nucleic acid sequence selected from the group consisting of SEQ ID NOs: 179-184.

In some embodiments, the heterologous protein is a genome-modifying protein. In some embodiments, the genome-modifying protein is a recombinant nuclease, a nickase, an integrase, reverse transcriptase, or a combination thereof. In some embodiments, the genome-modifying protein is a recombinant nuclease. In some embodiments, the genome-modifying protein is a nickase. In some embodiments, the genome-modifying protein is an integrase. In some embodiments, the genome-modifying protein is reverse transcriptase. In some embodiments, the genome-modifying protein is a zinc-finger nuclease (ZFN), a transcription-activator like effector nucleases (TALEN), or a CRISPR-associated (Cas) protein. In some embodiments, the genome-modifying protein is a Cas protein. In some embodiments, the genome-modifying protein is Cas9. In some embodiments, the genome-modifying protein is saCas9. In some embodiments, the genome-modifying protein is spCas9. In some embodiments, the genome-modifying protein is Cpf1.

In some embodiments, the heterologous protein is a tumor neoepitope. In some embodiments, the heterologous protein is a viral Spike(s) glycoprotein. In some embodiments, the heterologous protein is a protein from Zika virus, optionally Zika virus prM-E protein; tuberculosis; respiratory syncytial virus (RSV), optionally RSV fusion (RSV-F) protein; influenza virus, optionally influenza virus hemagglutinin (HA); rabies virus, optionally rabies virus glycoprotein (RABV-G); human cytolomegalovirus (CMV); hepatitis C virus; human immunodeficiency virus 1 (HIV-1), and Streptococcus. In some embodiments, the heterologous protein is an antibody or an antigen-binding fragment thereof.

In some embodiments, the lipid particle contains a guide RNA (gRNA) in the lumen. In some embodiments, the gRNA is a single guide RNA (sgRNA).

In some embodiments, the lipid particle is pseudotyped with a viral envelope glycoprotein. In some embodiments, the viral envelope glycoprotein is a VSV-G protein or a functional variant thereof. In some of any of the provided embodiments, wherein the VSV-G or a functional variant thereof comprises the amino acid sequence of SEQ ID NO: 199, or an amino acid sequence having at least 90%, 91%, 92%, 93%, 94%, 95%, 96%, 97%, 98%, or 99% sequence identity to the amino acid sequence of SEQ ID NO: 199. In some of any of the provided embodiments, the VSV-G or a functional variant thereof comprises the amino acid sequence of SEQ ID NO: 199.

In some embodiments, the viral envelope glycoprotein is a Cocal virus G protein or a functional variant thereof. In some embodiments, the viral envelope glycoprotein is an Alphavirus fusion protein (e.g. Sindbis virus) or a functional variant thereof. In some embodiments, the viral envelope glycoprotein is a Paramyxoviridae fusion protein (e.g., a Morbillivirus or a Henipavirus) or a functional variant thereof.

In some embodiments, the viral envelope glycoprotein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof. In some embodiments, the viral envelope glycoprotein is a Henipavirus fusion protein (e.g., Nipah virus, Hendra virus, Cedar virus, Kumasi virus, Mòjiāng virus, Langya virus) or a functional variant thereof.

In some embodiments, the viral envelope glycoprotein comprises one or more modifications to reduce binding to its native receptor. In some embodiments, the viral envelope glycoprotein comprises a Nipah virus F glycoprotein (NiV-F) or a biologically active portion thereof and a Nipah virus G glycoprotein (NiV-G) or a biologically active portion thereof. In some embodiments, the NiV-G or the biologically active portion thereof is a wild-type NiV-G protein or a functionally active variant or biologically active portion thereof. In some of any of the provided embodiments, the Niv-F or a biologically active portion thereof comprises the amino acid sequence set forth in SEQ ID NO: 145, or an amino acid sequence having at least 90%, 91%, 92%, 93%, 94%, 95%, 96%, 97%, 98%, or 99% sequence identity to the amino acid sequence set forth in SEQ ID NO: 145; and/or the Niv-G or a biologically active portion thereof comprises the amino acid sequence set forth in SEQ ID NO: 147, or an amino acid sequence having at least 90%, 91%, 92%, 93%, 94%, 95%, 96%, 97%, 98%, or 99% sequence identity to the amino acid sequence set forth in SEQ ID NO: 147. In some of any of the provided embodiments, the Niv-F or a biologically active portion thereof comprises the amino acid sequence set forth in SEQ ID NO: 145; and/or the Niv-G or a biologically active portion thereof comprises the amino acid sequence set forth in SEQ ID NO: 147.

In some embodiments, the NiV-G protein or the biologically active portion is truncated and lacks up to 40 contiguous amino acid residues at or near the N-terminus of the wild-type NiV-G protein.

In some embodiments, the NiV-G protein or the biologically active portion has a 5 amino acid truncation at or near the N-terminus of the wild-type NiV-G protein. In some embodiments, the NiV-G protein or the biologically active portion thereof has the amino acid sequence set forth in SEQ ID NO: 12, or a sequence of amino acids that exhibits at least at or about 80%, 85%, 90% or 95% sequence identity to the sequence set forth in SEQ ID NO: 12. In some embodiments, the NiV-G protein or the biologically active portion has a 10 amino acid truncation at or near the N-terminus of the wild-type NiV-G protein. In some embodiments, the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44. In some embodiments, the NiV-G protein or the biologically active portion has a 15 amino acid truncation at or near the N-terminus of the wild-type NiV-G protein. In some embodiments, the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45. In some embodiments, the NiV-G protein or the biologically active portion has a 20 amino acid truncation at or near the N-terminus of the wild-type NiV-G protein. In some embodiments, the NiV-G protein or the biologically active portion thereof has the amino acid sequence set forth in SEQ ID NO: 13, or a sequence of amino acids that exhibits at least at or about 80%, 85%, 90% or 95% sequence identity to the sequence set forth in SEQ ID NO: 13. In some embodiments, the NiV-G protein or the biologically active portion has a 25 amino acid truncation at or near the N-terminus of the wild-type NiV-G protein. In some embodiments, the NiV-G protein or the biologically active portion thereof has the amino acid sequence set forth in SEQ ID NO: 14, or a sequence of amino acids that exhibits at least at or about 80%, 85%, 90% or 95% sequence identity to the sequence set forth in SEQ ID NO: 14. In some embodiments, the NiV-G protein or the biologically active portion has a 30 amino acid truncation at or near the N-terminus of the wild-type NiV-G protein. In some embodiments, the NiV-G protein or the biologically active portion thereof has the amino acid sequence set forth in SEQ ID NO:43, or a sequence of amino acids that exhibits at least at or about 80%, 85%, 90% or 95% sequence identity to the sequence set forth in SEQ ID NO:43. In some embodiments, the NiV-G protein or the biologically active portion has a 34 amino acid truncation at or near the N-terminus of the wild-type NiV-G protein. In some embodiments, the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.

In some embodiments, the NiV-G-protein or the biologically active portion thereof is a mutant NiV-G protein that exhibits reduced binding to Ephrin B2 or Ephrin B3. In some embodiments, the mutant NiV-G protein or the biologically active portion contains one or more amino acid substitutions corresponding to amino acid substitutions selected from the group consisting of E501A, W504A, Q530A and E533A with reference to numbering set forth in SEQ ID NO: 4. In some embodiments, the mutant NiV-G protein or the biologically active portion contains the amino acid sequence set forth in SEQ ID NO: 17 or a sequence of amino acids that exhibits at least at or about 80%, 85%, 90% or 95% sequence identity to the sequence set forth in SEQ ID NO: 17. In some embodiments, the NiV-G protein or the biologically active portion contains the amino acid sequence set forth in SEQ ID NO: 18 or a sequence of amino acids that exhibits at least at or about 80%, 85%, 90% or 95% sequence identity to the sequence set forth in SEQ ID NO: 18.

In some embodiments, the NiV-F protein or the biologically active portion thereof is a wild-type NiV-F protein or is a functionally active variant or a biologically active portion thereof.

In some embodiments, the NiV-F protein or the biologically active portion thereof has a 20 amino acid truncation at or near the C-terminus of the wild-type NiV-F protein. In some embodiments, the NiV-F protein or the biologically active portion thereof has the sequence set forth in SEQ ID NO: 20 or a sequence of amino acids that exhibits at least at or about 80%, 85%, 90% or 95% sequence identity to the sequence set forth in SEQ ID NO: 20. In some embodiments, the NiV-F protein or the biologically active portion thereof contains: i) a 20 amino acid truncation at or near the C-terminus of the wild-type NiV-F protein; and ii) a point mutation on an N-linked glycosylation site. In some embodiments, the NiV-F protein or the biologically active portion thereof has the sequence set forth in SEQ ID NO: 15, or a sequence of amino acids that exhibits at least at or about 80%, 85%, 90% or 95% sequence identity to the sequence set forth in SEQ ID NO: 15.

In some embodiments, the NiV-F protein or the biologically active portion thereof has a 22 amino acid truncation at or near the C-terminus of the wild-type NiV-F protein. In some embodiments, the NiV-F protein or the biologically active portion thereof has the sequence set forth in SEQ ID NO: 16, 19, or 21 or a sequence of amino acids that exhibits at least at or about 80%, 85%, 90% or 95% sequence identity to the sequence set forth in SEQ ID NO: 16, 19, or 21. In some embodiments, the NiV-F protein or the biologically active portion thereof contains the amino acid sequence set forth in SEQ ID NO:21, or a sequence of amino acids that exhibits at least at or about 80%, 85%, 90% or 95% sequence identity to the sequence set forth in SEQ ID NO:21.

In some embodiments, the Niv-G protein contains the amino acid sequence set forth in SEQ ID NO: 17, and the Niv-F protein contains the amino acid sequence set forth in SEQ ID NO: 21.

In some embodiments, the lipid particle contains a targeting moiety. In some embodiments, the targeting moiety is selected from the group consisting of a CD3-binding agent, a CD8-binding agent, and a CD4-binding agent. In some embodiments, the targeting moiety is a CD3-binding agent. In some embodiments, the CD3-binding agent is an anti-CD3 antibody or an antigen-binding fragment. In some embodiments, the targeting moiety is a CD8-binding agent. In some embodiments, the CD8-binding agent is an anti-CD8 antibody or an antigen-binding fragment. In some embodiments, the targeting moiety is a CD4-binding agent. In some embodiments, the CD4-binding agent is an anti-CD4 antibody or an antigen-binding fragment.

In some embodiments, the targeting moiety is exposed on the surface of the lipid particle. In some embodiments, the targeting moiety is fused to a transmembrane domain incorporated into the bilayer of the lipid particle.

In some embodiments, the lipid particle is a retroviral vector or a retroviral-like particle. In some embodiments, the lipid particle is a retroviral vector. In some embodiments, the retroviral vector is replication-deficient. In some embodiments, the lipid particle is a retroviral-like particle. In some embodiments, the retroviral-like particle is replication-deficient.

In some embodiments, the lipid particle does not contain reverse transcriptase or does not contain reverse transcriptase activity. In some embodiments, the lipid particle does not contain a protein with reverse transcriptase activity. In some embodiments, the lipid particle does not contain reverse transcriptase. In some embodiments, the lipid particle contains non-functional reverse transcriptase. In some embodiments, the reverse transcriptase is mutated. In some embodiments, the retroviral vector contains a RNA that is a self-inactivating lentiviral vector genome. In some embodiments, the retroviral-like particle contains a RNA that is a self-inactivating lentiviral vector genome. In some embodiments, the retroviral vector or retroviral-like particle contains a RNA comprising a 3′LTR, and the 3′ LTR does not have a functional U3 domain. In some embodiments, the U3 domain comprises a deletion.

In some embodiments, the lipid particle is a retroviral particle, and the retroviral particle is a lentiviral particle. In some embodiments, the lipid particle is a retrovirus-like particle (VLP).

In some embodiments, the lipid bilayer is derived from a host cell. In some embodiments, the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

Also provided herein is a method of producing a lipid particle having a lipid bilayer enclosing a lumen and viral ribonucleic acid (RNA), including: (1) providing a host cell containing (a) a nucleic acid sequence selected from the group consisting of: a 5′ long terminal repeat (5′ LTR); a psi packaging signal sequence; a gag start codon; a RNA sequence encoding a heterologous protein; a 3′ long terminal repeat (3′ LTR); or a combination thereof; and (b) a nucleic acid sequence encoding a protein selected from the group consisting of gag, pol, rev, tat, a viral envelope glycoprotein, or a combination thereof; and (2) culturing the host cell under conditions to induce packaging of the lipid particle.

In some embodiments, a RNA sequence encoding a viral structural protein or a portion thereof is located between the gag start codon and the RNA sequence encoding a heterologous protein. In some embodiments, the gag start codon and the RNA sequence encoding a heterologous protein are part of the same RNA, and the RNA does not contain nucleotides between the gag start codon and the RNA sequence encoding a heterologous protein

Also provided herein is a method of producing a lipid particle having a lipid bilayer enclosing a lumen and viral ribonucleic acid (RNA), including: (1) providing a host cell containing (a) a RNA sequence encoding a heterologous protein and a viral structural protein or a portion thereof; and (b) a nucleic acid sequence encoding a protein selected from the group consisting of gag, pol, rev, tat, a viral envelope glycoprotein, or a combination thereof; and (2) culturing the host cell under conditions to induce packaging of the lipid particle.

In some embodiments, the RNA sequence encoding the viral structural protein or portion thereof is located 5′ to the RNA sequence encoding the heterologous protein. In some embodiments, a bicistronic element is located between the RNA sequence encoding the viral structural protein or portion thereof and the RNA sequence encoding the heterologous protein. In some embodiments, the bicistronic element is an internal ribosome entry site (IRES) element or a sequence encoding a 2A self-cleaving peptide. In some embodiments, the bicistronic element is a sequence encoding a 2A self-cleaving peptide, and the 2A self-cleaving peptide is T2A. In some embodiments. T2A comprises the sequence set forth in SEQ ID NO:76.

In some embodiments, the RNA sequence encodes, from a 5′ to 3′ direction: the viral structural protein or portion thereof, T2A, and the heterologous protein. In some embodiments, the viral structural protein is gag. In some embodiments, the RNA sequence encoding the viral structural protein or a portion thereof encodes an N-terminal portion of gag. In some embodiments, the RNA sequence encoding the viral structural protein or a portion thereof contains the sequence set forth in SEQ ID NO: 52. In some embodiments, the host cell contains a nucleic acid sequence that contains the sequence set forth in SEQ ID NO:77 and encodes the heterologous protein.

Also provide herein is a method of producing a lipid particle having a lipid bilayer enclosing a lumen and a viral transfer plasmid, including: (1) providing a host cell having (a) a nucleic acid sequence encoding a fusion protein comprising a viral matrix (MA) protein and a MS2 coat protein (MS2_{cp}); (b) a nucleic acid sequence encoding a heterologous protein; and (c) a nucleic acid sequence encoding a protein selected from the group consisting of gag, pol, Rev, Tat, a viral envelope glycoprotein, or a combination thereof; and (2) culturing the host cell under conditions to induce packaging of the lipid particle.

In some of any of the provided embodiments, the RNA binding protein is MS2 coat protein (MS2_{cp}). In some of any of the provided embodiments, MS2_{cp} comprises the sequence set forth in SEQ ID NO: 79. In some of any of the provided embodiments, the nucleic acid sequence encoding a heterologous protein comprises a MS2_{cp}-binding loop, optionally at or at least 2, 3, 4, 5, 6, 7, 8, 9, 10, 11, 12, 13, 14, 15, 16, 17, 18, 19, 20, 21, 22, 23, or 24 MS2_{cp}-binding loops. In some of any of the provided embodiments, the MS2_{cp}-binding loop comprises the RNA sequence transcribed from the nucleic acid set forth in SEQ ID NO: 185 or SEQ ID NO: 174. In some of any of the provided embodiments, the MS2_{cp}-binding loop comprises the RNA sequence set forth in SEQ ID NO: 208.

In some of any of the provided embodiments, the nucleic acid sequence encoding a heterologous protein comprises a plurality of MS2_{cp}-binding loops, and wherein the plurality of MS2_{cp}-binding loops comprises the RNA sequence transcribed from a nucleic acid sequence selected from the group consisting of SEQ ID NOs: 175-178. In some of any of the provided embodiments, the RNA binding protein is a lambda N protein (λN) or a functional variant thereof. In some of any of the provided embodiments, the λN or a functional variant thereof comprises the amino acid sequence of SEQ ID NO: 187 or an amino acid sequence having at least 90% or 95% sequence identity to the amino acid sequence of SEQ ID NO: 187. In some of any of the provided embodiments, the λN or a functional variant thereof comprises the amino acid sequence of SEQ ID NO: 187. In some of any of the provided embodiments, the λN or a functional variant thereof comprises the amino acid sequence of SEQ ID NO: 188 or an amino acid sequence having at least 90% or 95% sequence identity to the amino acid sequence of SEQ ID NO: 188. In some of any of the provided embodiments, the λN or a functional variant thereof comprises the amino acid sequence of SEQ ID NO: 188. In some of any of the provided embodiments, the nucleic acid sequence encoding a heterologous protein comprises a boxB binding site for binding to λN or a functional variant thereof, optionally at or at least 2, 3, 4, 5, 6, 7, 8, 9, 10, 11, 12, 13, 14, 15, 16, 17, 18, 19, 20, 21, 22, 23, or 24 boxB binding sites. In some of any of the provided embodiments, the boxB binding site comprises the RNA sequence transcribed from the nucleic acid set forth in SEQ ID NO: 186. In some of any of the provided embodiments, the nucleic acid sequence encoding a heterologous protein comprises a plurality of boxB binding sites for binding to λN or a functional variant thereof, and the plurality of boxB binding sites comprises the RNA sequence transcribed from a nucleic acid sequence selected from the group consisting of SEQ ID NOs: 179-184.

In some embodiments, the fusion protein contains, from a 5′ to 3′ direction: the viral MA protein and MS2_{cp}. In some embodiments, the nucleic acid sequence encoding a heterologous protein contains a MS2_{cp}-binding loop. In some embodiments, the nucleic acid sequence encoding a heterologous protein contains 12 MS2_{cp}-binding loops. In some embodiments, the nucleic acid sequence encoding a heterologous protein contains 24 MS2_{cp}-binding loops. In some embodiments, the viral MA protein is derived from human immunodeficiency virus (HIV). In some embodiments, the viral MA protein contains the sequence set forth in SEQ ID NO:78. In some embodiments, MS2_{cp} contains the sequence set forth in SEQ ID NO:79. In some embodiments, the fusion protein contains the sequence set forth in SEQ ID NO: 74.

Also provided herein is a method of producing a lipid particle having a lipid bilayer enclosing a lumen and a viral transfer plasmid, including:
(1) providing a host cell comprising (a) a nucleic acid sequence encoding a fusion protein containing viral matrix (MA) protein and a heterologous protein; and (b) a nucleic acid sequence encoding a protein selected from the group consisting of gag, pol, Rev, Tat, a viral envelope glycoprotein, or a combination thereof; and (2) culturing the host cell under conditions to induce packaging of the lipid particle.

Also provided herein is a method of producing a lipid particle having a lipid bilayer enclosing a lumen and a viral transfer plasmid, including:
(1) providing a host cell comprising (a) a nucleic acid sequence encoding a fusion protein containing a viral matrix (MA) protein and a MS2 coat protein (MS2_{cp}); (b) a nucleic acid sequence encoding a heterologous protein; and (b) a nucleic acid sequence encoding a protein selected from the group consisting of gag, pol, Rev. Tat, a viral envelope glycoprotein, or a combination thereof; and (2) culturing the host cell under conditions to induce packaging of the lipid particle.

In some embodiments, the fusion protein contains, from a 5′ to 3′ direction: the viral MA protein and the MS2_{cp}. In some embodiments, the fusion protein contains, from a 5′ to 3′ direction: the viral MA protein and the heterologous protein. In some embodiments, the nucleic acid sequence encoding a heterologous protein comprises a MS2_{cp}-binding loop, optionally 12 or 24 MS2_{cp}-binding loops. In some embodiments, the viral MA protein is derived from human immunodeficiency virus (HIV). In some embodiments, the viral MA protein contains the sequence set forth in SEQ ID NO:78. In some of any of the provided embodiments, MS2_{cp} comprises the sequence set forth in SEQ ID NO:79. In some of any of the provided embodiments, the fusion protein comprises the sequence set forth in SEQ ID NO: 74. In some embodiments, the viral envelope glycoprotein is VSV-G or a functional variant thereof.

Provided herein is a method of producing a lipid particle comprising a lipid bilayer enclosing a lumen and a viral transfer plasmid, comprising: (1) providing a host cell comprising (a) a nucleic acid sequence encoding a fusion protein comprising a viral envelope glycoprotein and an RNA binding protein; (b) a nucleic acid sequence encoding a heterologous protein; and (2) culturing the host cell under conditions to induce packaging of the lipid particle.

In some of any of the provided embodiments, the host cells further comprises a nucleic acid sequence encoding a protein selected from the group consisting of gag, pol. Rev, Tat, or a combination thereof. In some of any of the provided embodiments, the fusion protein comprises, from a 5′ to 3′ direction: the viral envelope glycoprotein and the RNA binding protein. In some of any of the provided embodiments, wherein the RNA binding protein is a MS2 coat protein (MS2_{cp}). In some of any of the provided embodiments, MS2_{cp} comprises the sequence set forth in SEQ ID NO:79. In some of any of the provided embodiments, the nucleic acid sequence encoding a heterologous protein comprises a MS2_{cp}-binding loop, optionally at or at least 12 or 24 MS2_{cp}-binding loops. In some of any of the provided embodiments, the MS2_{cp}-binding loop comprises the RNA sequence transcribed from the nucleic acid set forth in SEQ ID NO: 185 or SEQ ID NO: 174. In some of any of the provided embodiments, the MS2_{cp}-binding loop comprises the RNA sequence set forth in SEQ ID NO: 208. In some of any of the provided embodiments, the nucleic acid sequence encoding a heterologous protein comprises a plurality of MS2_{cp}-binding loops, and wherein the plurality of MS2_{cp}-binding loops comprises the RNA sequence transcribed from a nucleic acid sequence selected from the group consisting of SEQ ID NOs: 175-178. In some of any of the provided embodiments, the viral envelope glycoprotein is derived from human immunodeficiency virus (HIV). In some of any of the provided embodiments, the RNA binding protein is a lambda N protein (2N) or a functional variant thereof.

In some of any of the provided embodiments, the nucleic acid sequence encoding a heterologous protein comprises a boxB binding site for binding to λN or a functional variant thereof, optionally at or at least 2, 3, 4, 5, 6, 7, 8, 9, 10, 11, 12, 13, 14, 15, 16, 17, 18, 19, 20, 21, 22, 23, or 24 boxB binding sites. In some of any of the provided embodiments, the boxB binding site comprises the RNA sequence transcribed from the nucleic acid set forth in SEQ ID NO: 186. In some of any of the provided embodiments, the nucleic acid sequence encoding a heterologous protein comprises a plurality of boxB binding sites for binding to λN or a functional variant thereof, and the plurality of boxB binding sites comprises the RNA sequence transcribed from a nucleic acid sequence selected from the group consisting of SEQ ID NOs: 179-184.

Provided herein is a method of producing a lipid particle comprising a lipid bilayer enclosing a lumen and a viral transfer plasmid, comprising (1) providing a host cell comprising (a) a nucleic acid sequence encoding a fusion protein comprising viral matrix (MA) protein and a heterologous protein; and (b) a nucleic acid sequence encoding a protein selected from the group consisting of gag, pol, Rev, Tat, a viral envelope glycoprotein, or a combination thereof; and (2) culturing the host cell under conditions to induce packaging of the lipid particle.

In some of any of the provided embodiments, the fusion protein comprises, from a 5′ to 3′ direction: the viral MA protein and the heterologous protein. In some of any of the provided embodiments, the viral MA protein is derived from human immunodeficiency virus (HIV). In some of any of the provided embodiments, the viral MA protein comprises the sequence set forth in SEQ ID NO: 78. In some of any of the provided embodiments, the viral envelope glycoprotein is VSV-G.

In some embodiments, the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

In some embodiments, the nucleic acid sequence in (b) contains a 5′ promoter. In some embodiments, the nucleic acid sequence in (c) contains a 5′ promoter. In some embodiments, the promoter is a cytomegalovirus (CMV) promoter.

Provided herein is a DNA sequence encoding a viral matrix (MA) protein, an RNA binding protein (MS2_{cp}), and a cleavage site between the portions of the DNA sequence encoding the MA protein and the RNA binding protein. In some of any of the provided embodiments, the DNA encodes a fusion protein comprising, from 5′ to 3′, the viral MA protein and RNA binding protein. In some of any of the provided embodiments, the RNA binding protein is a MS2 coat protein (MS2_{cp}). In some of any of the provided embodiments, MS2_{cp} comprises the sequence set forth in SEQ ID NO:79. In some of any of the provided embodiments, the RNA binding protein is a lambda N protein (λN) or a functional variant thereof. In some of any of the provided embodiments, the λN or a functional variant thereof comprises the amino acid sequence of SEQ ID NO: 187 or 188. In some of any of the provided embodiments, the encoded viral MA protein comprises the sequence set forth in SEQ ID NO:78. In some of any of the provided embodiments, the nucleic acid sequence set forth in any one of SEQ ID NOs: 62, 150, 153, and 154.

Provided herein is a DNA sequence encoding a viral matrix (MA) protein, a MS2 coat protein (MS2_{cp}), and a cleavage site between the portions of the DNA sequence encoding the MA protein and the MS2_{cp}. In some of any of the provided embodiments, the DNA encodes a fusion protein comprising, from 5′ to 3′, the viral MA protein and MS2_{cp}.

Also provided herein is a lipid particle produced by any of the methods herein.

Also provided herein is a composition comprising any lipid particle provided herein.

Also provided herein is a method of introducing a heterologous protein into a target cell, the method including contacting the target cell with a lipid particle or a composition provided herein. In some embodiments, the contacting is in vitro or ex vivo. In some embodiments, the method is in vivo.

Also provided herein is a method of genetically engineering a target cell, the method including contacting the target cell with a lipid particle or a composition provided herein. In some embodiments, the contacting is in vitro or ex vivo. In some embodiments, the method is in vivo.

Also provided herein is a deoxyribonucleic acid (DNA) sequence encoding a gag start codon and a heterologous protein. In some embodiments, the contacting is in vitro or ex vivo. In some embodiments, the method is in vivo. In some embodiments, the DNA sequence encodes a viral structural protein or a portion thereof, wherein the portion of the DNA sequence encoding the viral structural protein is located between the portions of the DNA sequence encoding the gag start codon and the heterologous protein. In some embodiments, the DNA sequence encodes a bicistronic element, wherein the portion of the DNA sequence encoding the bicistronic element is located between the portions of the DNA sequence encoding the viral structural protein or a portion thereof and the heterologous protein. In some embodiments, the bicistronic element is an internal ribosome entry site (IRES) element or a sequence encoding a 2A self-cleaving peptide. In some embodiments, the 2A self-cleaving peptide is T2A. In some embodiments, T2A contains the sequence set forth in SEQ ID NO:76. In some embodiments, the DNA sequence encodes from a 5′ to 3′ direction: the viral structural protein or portion thereof. T2A, and the heterologous protein.

In some embodiments, the viral structural protein is gag. In some embodiments, the DNA sequence encodes an N-terminal portion of gag. In some embodiments, the N-terminal portion of gag contains the sequence set forth in SEQ ID NO:52. In some embodiments, the DNA sequence encodes the sequence set forth in SEQ ID NO:77 and the heterologous protein. In some embodiments, the DNA sequence does not comprise nucleotides between the encoded gag start codon and the encoded heterologous protein. In some embodiments, the DNA sequence contains a promoter. In some embodiments, the promoter is a cytomegalovirus (CMV) promoter.

Also provide herein is a DNA sequence encoding a viral matrix (MA) protein, a MS2 coat protein (MS2_{cp}), and a cleavage site between the portions of the DNA sequence encoding the MA protein and the MS2_{cp}.

In some embodiments, the DNA sequence encodes a fusion protein containing, from 5′ to 3′, the viral MA protein and MS2_{cp}. In some embodiments, the encoded MS2_{cp} contains a MS2_{cp}-binding loop. In some embodiments, the encoded MS2_{cp} contains 12 MS2_{cp}-binding loops. In some embodiments, the encoded MS2_{cp} contains 24 MS2_{cp}-binding loops. In some embodiments, the encoded viral MA protein is derived from human immunodeficiency virus (HIV). In some embodiments, the encoded viral MA protein contains the sequence set forth in SEQ ID NO:78. In some embodiments, the encoded MS2_{cp} contains the sequence set forth in SEQ ID NO:79. In some embodiments, the encoded fusion protein contains the sequence set forth in SEQ ID NO:74.

Provided herein is a DNA sequence encoding a viral envelope glycoprotein, an RNA binding protein, and a cleavage site between the portions of the DNA sequence encoding the viral envelope glycoprotein and the RNA binding protein. In some of any of the provided embodiments, the fusion protein comprises, from a 5′ to 3′ direction: the viral envelope glycoprotein and the RNA binding protein. In some of any of the provided embodiments, the viral envelope glycoprotein is a VSV-G protein or a functional variant thereof. In some of any of the provided embodiments, the viral envelope glycoprotein is derived from human immunodeficiency virus (HIV). In some of any of the provided embodiments, the RNA binding protein is a MS2 coat protein (MS2_{cp}). In some of any of the provided embodiments, MS2_{cp} comprises the sequence set forth in SEQ ID NO:79. In some of any of the provided embodiments, the RNA binding protein is a lambda N protein (λN) or a functional variant thereof. In some of any of the provided embodiments, the λN or a functional variant thereof comprises the amino acid sequence set forth in SEQ ID NO: 187 or 188. In some of any of the provided embodiments, comprising a nucleic acid sequence selected from the group consisting of SEQ ID NOs: 62 and 150-156. In some of any of the provided embodiments, comprising a nucleic acid sequence encoding the amino acid sequence set forth in SEQ ID NO: 157 or 158. In some of any of the provided embodiments, comprising a nucleic acid sequence encoding the amino acid sequence set forth in SEQ ID NO: 192 or 193. In some of any of the provided embodiments, comprising the nucleic acid sequence set forth in SEQ ID NO: 155 or 156. In some of any of the provided embodiments, comprising a nucleic acid sequence encoding the amino acid sequence set forth in SEQ ID NO: 161 or 162. In some of any of the provided embodiments, comprising a nucleic acid sequence encoding the amino acid sequence set forth in SEQ ID NO: 196 or 197.

Also provided herein is a DNA sequence encoding a viral matrix (MA) protein and a heterologous protein. In some embodiments, the DNA sequence encodes a fusion protein containing, from 5′ to 3′, the viral MA protein and the heterologous protein. In some embodiments, the encoded viral MA protein is derived from human immunodeficiency virus (HIV). In some embodiments, the encoded viral MA protein contains the sequence set forth in SEQ ID NO:78.

In some embodiments, the heterologous protein is a genome-modifying protein. In some embodiments, the genome-modifying protein is a recombinant nuclease, a nickase, an integrase, reverse transcriptase, or a combination thereof. In some embodiments, the genome-modifying protein is a recombinant nuclease. In some embodiments, the genome-modifying protein is a nickase. In some embodiments, the genome-modifying protein an integrase. In some embodiments, the genome-modifying protein is reverse transcriptase. In some embodiments, the genome-modifying protein comprises a zinc-finger nuclease (ZFN), a transcription-activator like effector nucleases (TALEN), or a CRISPR-associated (Cas) protein. In some embodiments, the genome-modifying protein is a Cas protein. In some embodiments, the genome-modifying protein is Cas9. In some embodiments, the genome-modifying protein is saCas9. In some embodiments, the genome-modifying protein is spCas9. In some embodiments, the genome-modifying protein is Cpf1.

In some embodiments, the heterologous protein is a tumor neoepitope. In some embodiments, the heterologous protein is a viral Spike(s) glycoprotein. In some embodiments, the heterologous protein is a protein from Zika virus, optionally Zika virus prM-E protein; tuberculosis; respiratory syncytial virus (RSV), optionally RSV fusion (RSV-F) protein; influenza virus, optionally influenza virus hemagglutinin (HA); rabies virus, optionally rabies virus glycoprotein (RABV-G); human cytolomegalovirus (CMV); hepatitis C virus; human immunodeficiency virus 1 (HIV-1), and Streptococcus. In some embodiments, the heterologous protein is an antibody or an antigen-binding fragment thereof.

Also provided herein is a vector containing a DNA sequence provided herein.

Also provided herein is a mammalian cell containing a DNA sequence provided herein. Also provided herein is a mammalian cell containing a vector provided herein.

In some embodiments, the mammalian cell contains viral nucleic acid, wherein the viral nucleic acid lacks one or more genes involved in viral replication. In some embodiments, the viral nucleic acid contains: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3); a nucleic acid encoding a viral envelope protein; and/or a nucleic acid encoding a viral packaging protein selected from one or more of gag, pol, rev and tat.

In some embodiments, the mammalian cell contains a RNA sequence encoding a heterologous protein. In some embodiments, the mammalian cell contains a guide RNA (gRNA).

Also provided herein is a transfer plasmid having a promoter operably linked to a RNA sequence encoding a gag protein or portion thereof comprising at least a gag start codon; a RNA sequence encoding a heterologous protein that is linked to the RNA sequence encoding a gag protein or portion thereof; and a 3′ long terminal repeat (3′ LTR).

In some of any of the provided embodiments, the retroviral packaging sequence comprises a mutation in a major splice donor site. In some of any of the provided embodiments, the major splice donor site is a major splice donor site contained in SL2 of HIV psi. In some of any of the provided embodiments, the mutation is a mutation that inhibits splicing at the major splice donor site. In some of any of the provided embodiments, the mutated major splice donor site comprises a mutation that prevents splicing at the major splice donor site.

Also provided herein is a transfer plasmid having a promoter operably linked to a ribonucleic acid (RNA), wherein the RNA comprises, from 5′ to 3′: a 5′ long terminal repeat (5′ LTR); a gag 5′ untranslated region (UTR) or portion thereof comprising at least three nucleotides; a RNA sequence encoding a heterologous protein that is linked to the gag 5′ UTR or a portion thereof; and a 3′ long terminal repeat (3′ LTR).

Also provided herein is a transfer plasmid having a promoter operably linked to a ribonucleic acid (RNA), wherein the RNA comprises, from 5′ to 3′: a 5′ long terminal repeat (5′ LTR); a retroviral packaging sequence; a gag start codon; a RNA sequence encoding a heterologous protein; and a 3′ long terminal repeat (3′ LTR).

Also provided herein is a transfer plasmid having a promoter operably linked to a nucleic acid sequence encoding a fusion protein containing a viral matrix (MA) protein and a MS2 coat protein (MS2_{cp}).

Also provided herein is a transfer plasmid having a promoter operably linked to a nucleic acid sequence encoding a viral matrix (MA) protein and a heterologous protein.

In some embodiments, the transfer plasmid is a lentiviral transfer plasmid.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### BRIEF DESCRIPTION OF THE DRAWINGS

FIG. 1 depicts construct 5 (pMA2 N-term GAG-T2A-Cre without a promoter).

FIG. 2 depicts an exemplary packaging process for delivery of genome modifying agents using genomic viral mRNA.

FIG. 3 shows the level of tdTomato protein expression in HEK293 tdTomato reporter cells following titration with 0.5 or 5.0 μL of lentiviral particles containing a plasmid encoding Cre recombinase.

FIG. 4 shows representative images of the expression of tdTomato protein in the spleen of lox-STOP-lox-tdTomato cre reporter mice treated with virus-like particles (VLPs) delivering Cre recombinase.

FIG. 5 depicts an exemplary packaging process for delivery of genome modifying agents by tethering RNA to viral particles.

FIG. 6 shows the level of tdTomato protein expression in HEK293 tdTomato reporter cells following titration with 0.5 or 5.0 mL of lentiviral particles or VLPs delivering a MA-MS2_{cp}-CA gagpol fusion protein that tethers co-expressed Cre-encoding mRNA to the interior of the particle.

FIG. 7 shows the viral titer achieved by packaging of gesicles with Cre-encoding mRNA and a transgene encoding a membrane attachment domain attached to MS2_{cp}.

FIG. 8 shows the expression of tdTomato protein in HEK293 tdTomato reporter cells following incubation with 1, 10, or 100 μL of gesicles with Cre-encoding mRNA.

FIG. 9 depicts an exemplary packaging process for delivery of genome modifying agents by tethering proteins to viral particles.

FIG. 10 depicts the viral titer of gesicles containing fusion proteins containing a membrane attachment domain and Cre recombinase.

FIG. 11 shows the expression of tdTomato protein in HEK293 tdTomato reporter cells following incubation with 1, 10, or 100 μL of gesicles packaging construct 25.

FIG. 12 depicts the percentage of tdTomato expressing cells in the bone marrow, peripheral blood, and spleen of mice 10 days following injection with gesicles, LLV, or VLP delivering the specified cargo.

FIG. 13 shows the level of tdTomato protein expression in lox-STOP-lox-tdTomato cre reporter mice administered gesicles with MA fused with Cre after 17 days post injection.

FIG. 14 shows representative flow plots of activated T cells isolated from lox-STOP-lox-tdTomato Cre reporter mice that were administered gesicles with MA fused with Cre (construct 25) or a lentivirus with Cre (construct 1).

FIG. 15 shows a flow chart of HEK293 cells with a tdTomato red Cre reporter treated with viral like particles (VLPs) that deliver Cre and have either an unmutated HIV major splice donor (construct 5) or a mutated major splice donor (construct 26) with the VLPs made with a plasmid driving VSV-G expression.

FIG. 16 shows a flow chart of cells treated with VLPs that deliver EGFP and have either an unmutated HIV major splice donor (construct 27) or a mutated major splice donor (construct 28) with the VLPs made with a plasmid driving VSV-G expression.

FIG. 17 shows a flow chart of HEK293 cells with a tdTomato red Cre reporter treated with VLPs that deliver Cre and have either an unmutated HIV major splice donor (construct 29) or a mutated major splice donor (construct 30) with the VLPs made with a plasmid driving expression of NiV-F and NiV-G.

FIG. 18 shows a flow chart of 293T cells treated with VLPs that deliver EGFP and have either an unmutated HIV major splice donor (construct 31) or a mutated major splice donor (construct 32) with the VLPs made with a plasmid driving NiV-F and NiV-G expression.

FIG. 19 shows a flow chart of 293T cells treated with gesicles that deliver EGFP as an mRNA with MS2_{cp} stem loops (construct 33) or delivers EGFP as a protein fused with the MA protein (construct 34).

FIG. 20 depicts the titer of gesicles formed when testing varying configurations of either MS2_{cp} stem loops or boxB binding sites designated along the X axis as SLs with different configurations of binding proteins within the gesicles designated as different constructs with over expression of VSV-G from a separate plasmid.

FIG. 21 depicts the titer of gesicles formed when testing varying configurations of either MS2_{cp} stem loops or boxB binding sites designated along the X axis with different configurations of binding proteins that have VSV-G directly fused to the binding protein to test gesicle formation without expression of VSV-G from a separate plasmid.

FIG. 22 depicts the titer of gesicles formed using constructs from FIG. 20 with different ratios of RNA binding protein, Cre-mRNA, and VSV-G envelope plasmids.

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably +0.1% from the specified value, as such variations are appropriate to perform the disclosed methods.

As used herein, “fusosome” refers to a particle containing a bilayer of amphipathic lipids enclosing a lumen or cavity and a fusogen that interacts with the amphipathic lipid bilayer. In embodiments, the fusosome comprises a nucleic acid. In some embodiments, the fusosome is a membrane enclosed preparation. In some embodiments, the fusosome is derived from a source cell. In some embodiments, the fusosome is derived from a vector, such as a viral vector (e.g., a lentiviral vector).

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In embodiments, the fusogen comprises one or more sequence alterations outside of the targeting moiety relative to the naturally-occurring form of the fusogen, e.g., in a transmembrane domain, fusogenically active domain, or cytoplasmic domain.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “heterologous agent” as used herein with reference to a viral vector, refers to an agent that is neither comprised by nor encoded in the corresponding wild-type virus or fusogen made from a corresponding wild-type source cell. In some embodiments, the heterologous agent does not naturally exist, such as a protein or nucleic acid that has a sequence that is altered (e.g., by insertion, deletion, or substitution) relative to a naturally occurring protein. In some embodiments, the heterologous agent does not naturally exist in the source cell. In some embodiments, the heterologous agent exists naturally in the source cell but is exogenous to the virus. In some embodiments, the heterologous agent does not naturally exist in the recipient cell. In some embodiments, the heterologous agent exists naturally in the recipient cell, but is not present at a desired level or at a desired time. In some embodiments, the heterologous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, “operably linked” or “operably associated” includes reference to a functional linkage of at least two sequences. For example, operably linked includes linkage between a promoter and a second sequence, wherein the promoter sequence initiates and mediates transcription of the DNA sequence corresponding to the second sequence. Operably associated includes linkage between an inducing or repressing element and a promoter, wherein the inducing or repressing element acts as a transcriptional activator of the promoter.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes a heterologous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Y)), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, rev, and env.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “treat.” “treating.” or “treatment” refer to ameliorating a disease or disorder, e.g., slowing or arresting or reducing the development of the disease or disorder, e.g., a root cause of the disorder or at least one of the clinical symptoms thereof.

As used herein, the terms “effective amount” and “pharmaceutically effective amount” refer to a nontoxic but sufficient amount of an agent or drug to provide the desired biological result. That result can be reduction and/or alleviation of the signs, symptoms, or causes of a disease or disorder, imaging or monitoring of an in vitro or in vivo system (including a living organism), or any other desired alteration of a biological system. An appropriate effective amount in any individual case may be determined by one of ordinary skill in the art using routine experimentation.

### Lipid Particles and Methods for Generating the Same

Provided herein is a lipid particle comprising a lipid bilayer enclosing a lumen and containing a heterologous agent (e.g. a heterologous protein, a heterologous nucleic acid per se, or a nucleic acid encoding a heterologous protein). The lipid particle can be used for delivery of the heterologous agent to a cell. In some embodiments, the heterologous agent is heterologous RNA per se, i.e. RNA not encoding a heterologous protein, for example a guide RNA (gRNA). In some embodiments, the heterologous agent is a RNA sequence encoding a heterologous protein. In some embodiments, the heterologous agent is a fusion protein. In some embodiments, the fusion protein dissociates within the lipid particle, such as by cleavage, to produce two separate polypeptides. In some embodiments, at least one of the dissociated polypeptides is a heterologous protein. In some embodiments, the heterologous protein is a genome-modifying protein (e.g., a recombinant nuclease).

Also provided are methods of producing the lipid particles and compositions comprising the lipid particles. Also provided herein are methods of delivering any of the provided lipid particles to a cell (e.g., a target cell) and methods of genetically engineering a cell. In some embodiments, a lipid particle is introduced into a cell, such as by contacting the cell with the lipid particle. In some embodiments the contacting is in vitro or ex vivo. In some embodiments, the contacting is in vivo in a subject.

In some embodiments, the provided lipid particles exhibit fusogenic activity, which is mediated for example by a viral envelope protein or portion thereof that facilitates merger or fusion of the two lumens of the lipid particle and the target cell membrane. Thus, among provided lipid particles are fusosomes. In some embodiments, the fusosome comprises a naturally derived bilayer of amphipathic lipids with a viral envelope glycoprotein as a fusogen. In some embodiments, the fusosome comprises (a) a lipid bilayer, (b) a lumen (e.g., comprising cytosol) surrounded by the lipid bilayer; and (c) a fusogen that is exogenous or overexpressed relative to the source cell. In some embodiments, the viral envelope protein is vesicular stomatitis virus G (VSV-G) protein, such that the lipid particle is pseudotyped with VSV-G.

In some embodiments, the viral envelope protein is a viral protein, such as a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatitis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa california multiple NPV (AcMNPV) gp64), Baboon endogenous retrovirus envelope glycoprotein (BaEV), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic post fusion conformation with a signature trimer of α-helical hairpins with a central coiled-coil structure. Class I viral fusion proteins include proteins having a central post fusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises α helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi: 10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi: 10.1016/j.devcel.2007.12.008).

Additional exemplary fusogens are disclosed in U.S. Pat. No. 9,695,446, US 2004/0028687, U.S. Pat. No. 6,416,997. U.S. Pat. No. 7,329,807, US 2017/0112773, US 2009/0202622, WO 2006/027202, and US 2004/0009604, the entire contents of all of which are hereby incorporated by reference.

In some embodiments, the fusogen is a poxviridae fusogen.

In some embodiments the fusogen is a paramyxovirus fusogen. In some embodiments, the fusogen may be an envelope glycoprotein G, H HN and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In particular embodiments, the fusogen is glycoprotein GP64 of baculovirus, glycoprotein GP64 variant E45K/T259A.

In some embodiments, the fusogen is a hemagglutinin-neuraminidase (HN) and fusion (F) proteins (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry to cells via the F protein. In some embodiments, the fusogen is a F and/or HN protein from the murine parainfluenza virus type 1 (See e.g., U.S. Pat. No. 10,704,061).

In some embodiments the fusogen is a paramyxovirus fusogen. In some embodiments, the fusogen may be or an envelope glycoprotein G, H and/or an F protein of the Paramyxoviridae family. In some embodiments the fusogen contains a Nipah virus protein F, a measles virus F protein, a canine distemper virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi: 10.1038/nature12343), myomixer (nature.com/nature/journal/v499/n7458/full/nature12343.html, doi: 10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells, any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

In some embodiments, the lipid particle includes a naturally derived bilayer of amphipathic lipids that encloses lumen or cavity. In some embodiments, the lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen includes cytosol. In some embodiments, the cytosol contains cellular components present in a source cell. In some embodiments, the cytosol does not contain components present in a source cell. In some embodiments, the lumen is a cavity. In some embodiments, the cavity contains an aqueous environment. In some embodiments, the cavity does not contain an aqueous environment.

In some embodiments, the lipid particle can be a viral-based particles, such as a viral particle (e.g., a retroviral particle such as a retroviral or lentiviral particle) or a virus-like particle (VLP) such as a retrovirus-like particle or a lentivirus-like particle. In some embodiments, the lipid is a retroviral particle, such as a lentiviral particle. In some embodiments, the lipid particle is a virus-like particle (VLP). In some embodiments, the lipid particle is a cell-based particle, such as a gesicle. In some embodiments, the lipid particle is a gesicle.

In some aspects, the lipid bilayer is derived from a source cell during a process to produce a lipid-containing particle. Exemplary methods for producing lipid-containing particles are described herein. In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In particular embodiments, the lipid particle is virally derived. In some embodiments, the lipid particle can be a viral-based particle, such as a viral vector particle (e.g. retroviral or lentiviral vector particle) or a virus-like particle (e.g. a retroviral- or lentiviral-like particle). In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In particular embodiments, the lipid particle is not virally derived. In some embodiments, the lipid particle is cell-based particle. For example, in some embodiments, the lipid particle is a nanovesicle, such as a gesicle. In some embodiments, a gesicle is a VSV-G induced nanovesicle produced by overexpression of VSV-G in a host cell (Mangeot et al., Mol Ther (2011) 19 (9): 1656-66).

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In particular embodiments, a heterologous agent, such as a polynucleotide or polypeptide, is encapsulated within the lumen of a lipid particle. Embodiments of provided lipid particles may have various properties that facilitate delivery of a payload, such as, e.g., a desired transgene or heterologous agent, to a target cell. The heterologous agent may be a polynucleotide or a polypeptide. In some embodiments, a lipid particle provided herein is administered to a subject, e.g., a mammal, e.g., a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains a nucleic acid sequence (polynucleotide) encoding a heterologous agent or a polypeptide heterologous agent for treating the disease or condition.

The lipid particles can include spherical particles or can include particles of elongated or irregular shape.

In some embodiments, a composition of particles can be assessed for one or more features related to their size, including diameter, range of variation thereof above and below an average (mean) or median value of the diameter, coefficient of variation, polydispersity index or other measure of size of particles in a composition. Various methods for particle characterization can be used, including, but not limited to, laser diffraction, dynamic light scattering (DLS; also known as photon correlation spectroscopy) or image analysis, such as microscopy or automated image analysis.

In some embodiments, the provided lipid particle has a diameter of, or the average (mean) diameter of particles in a composition is, less than about 3 μm, less than about 2 μm, less than about 1 μm, less than about 900 nm, less than about 800 nm, less than about 700 nm, less than about 600 nm, less than about 500 m, less than about 400 nm, less than about 300, less than about 200 nm, less than about 150 nm, less than about 100 nm, less than about 50 nm, or less than about 20 nm. In some embodiments, the lipid particle has a diameter of, or the average (mean) diameter of particles in a composition is, less than about 400 nm. In another embodiment, the lipid particle has a diameter of, or the average (mean) diameter of particles in a composition is, less than about 150 nm. In some embodiments, the lipid particle has a diameter of, or the average (mean) diameter of particles in a composition is, between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments the median particle diameter in a composition of particles is between at or about 10 nm and at or about 1000 nM, between at or about 25 nm and at or about 500 nm, between at or about 40 nm and at or about 300 nm, between at or about 50 nm and at or about 250 nm, between at or about 60 nm and at or about 225 nm, between at or about 70 nm and at or about 200 nm, between at or about 80 nm and at or about 175 nm, or between at or about 90 nm and at or about 150 nm.

In some embodiments, 90% of the lipid particles in a composition fall within 50% of the median diameter of the lipid particles. In some embodiments, 90% of the lipid particles in a composition fall within 25% of the median diameter of the lipid particles. In some embodiments, 90% of the lipid particles in a composition fall within 20% of the median diameter. In some embodiments, 90% of the lipid particles in a composition fall within 15% of the median diameter of lipid particles. In some embodiments, 90% of the lipid particles in a composition fall within 10% of the median diameter of the lipid particles.

In some embodiments, 75% of the lipid particles in a composition fall within +/−2 or +/−1 St Dev standard deviations (St Dev) of the mean diameter of lipid particles. In some embodiments, 80% of the lipid particles in a composition fall within +/−2 St Dev or +/−1 St Dev of the mean diameter of lipid particles. In some embodiments, 85% of the lipid particles in a composition fall within +/−2 St Dev or +/−1 St Dev of the mean diameter of lipid particles. In some embodiments, 90% of the lipid particles in a composition fall within +/−2 St Dev or +/−1 St Dev of the mean diameter of lipid particles. In some embodiments, 95% of the lipid particles in a composition fall within +/−2 St Dev or +/−1 St Dev of the mean diameter of lipid particles.

In some embodiments, the lipid particles have an average hydrodynamic radius, e.g. as determined by DLS, of about 100 nm to about two microns. In some embodiments, the lipid particles have an average hydrodynamic radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the lipid particles have an average geometric radius, e.g. as determined by a multi-angle light scattering, of about 100 nm to about two microns. In some embodiments, the lipid particles have an average geometric radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the coefficient of variation (COV) (i.e. standard deviation divided by the mean) of a composition of lipid particles is less than at or about 30%, less than at or about 25%, less than at or about 20%, less than at or about 15%, less than at or about 10% or less than at or about 5%.

In some embodiment, provided compositions of lipid particles are characterized by their polydispersity index, which is a measure of the size distribution of the particles wherein values between 1 (maximum dispersion) and 0 (identical size of all of the particles) are possible. In some embodiments, compositions of lipid particles provided herein have a polydispersity index of between at or about 0.05 and at or about 0.7, between at or about 0.05 and at or about 0.6, between at or about 0.05 and at or about 0.5, between at or about 0.05 and at or about 0.4, between at or about 0.05 and at or about 0.3, between at or about 0.05 and at or about 0.2, between at or about 0.05 and at or about 0.1, between at or about 0.1 and at or about 0.7, between at or about 0.1 and at or about 0.6, between at or about 0.1 and at or about 0.5, between at or about 0.1 and at or about 0.4, between at or about 0.1 and at or about 0.3, between at or about 0.1 and at or about 0.2, between at or about 0.2 and at or about 0.7, between at or about 0.2 and at or about 0.6, between at or about 0.2 and at or about 0.5, between at or about 0.2 and at or about 0.4 between at or about 0.2 and at or about 0.3, between at or about 0.3 and at or about 0.7, between at or about 0.3 and at or about 0.6, between at or about 0.3 and at or about 0.5, between at or about 0.3 and at or about 0.4, between at or about 0.4 and at or about 0.7, between at or about 0.4 and at or about 0.6, between at or about 0.4 and at or about 0.5, between at or about 0.5 and at or about 0.7, between at or about 0.5 and at or about 0.6, or between at or about 0.6 and at or about 0.7. In some embodiments, the polydispersity index is less than at or about 0.05, less than at or about 0.1, less than at or about 0.15, less than at or about 0.2, less than at or about 0.25, less than at or about 0.3, less than at or about 0.4, less than at or about 0.5, less than at or about 0.6 or less than at or about 0.7.

Various lipid particles are known, any of which can be generated in accord with the provided embodiments. Non-limiting examples of lipid particles include any as described in, or contain features as described in, International published PCT Application No. WO 2017/095946; WO 2017/095944; WO 2017/095940; WO 2019/157319; WO 2018/208728; WO 2019/113512; WO 2019/161281; WO 2020/102578; WO 2019/222403; WO 2020/014209; WO 2020/102485; WO 2020/102499; WO 2020/102503; WO 2013/148327; WO 2017/182585; WO 2011/058052; or WO 2017/068077, each of which are incorporated by reference in their entirety.

### A. Methods of Providing Lipid Particles

Provided herein are lipid particles comprising a heterologous agent. In some embodiments, the heterologous agent is a heterologous protein. In some embodiments, a lipid particle provided herein comprises a lipid bilayer enclosing a lumen. In some embodiments, the lipid particle further comprises viral nucleic acid encoding for a heterologous protein. In some embodiments, the lipid particle further comprises a fusion protein and an RNA sequence encoding a heterologous protein, wherein at least a portion of the fusion protein is integrated into the lipid bilayer of the lipid particle and the RNA sequence binds to at least a portion of the fusion protein. In some embodiments, the lipid particle further comprises a fusion protein comprising a heterologous protein, wherein at least a portion of the fusion protein is integrated into the lipid bilayer of the lipid particle.

### 1. Viral Genome

Provided herein are lipid particles comprising a lipid bilayer enclosing a lumen and a ribonucleic acid (RNA). In some embodiments, the RNA contains retroviral translational control elements. In some embodiments, the RNA contains retroviral packaging elements. In some embodiments, the lipid particle does not have reverse transcriptase activity. Thus, in some embodiments, the RNA is viral genomic RNA encoding a heterologous protein, such that the viral genomic RNA allows for the production of the heterologous protein, which can be delivered to a cell of interest by the lipid particle.

Provided herein is a lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a RNA sequence encoding a gag protein or portion thereof comprising at least a gag start codon; a RNA sequence encoding a heterologous protein that is operably linked to the RNA sequence encoding a gag protein or portion thereof; and a poly-A tail, wherein each of the R element of the 5′ LTR, the U5 element of the 5′ LTR, and the RNA sequence encoding a gag protein or portion thereof is retroviral. In some embodiments, the RNA comprises a retroviral packaging sequence that is 3′ to the 5′ LTR.

Also provided herein is a lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a gag 5′ untranslated region (UTR) or portion thereof comprising at least three nucleotides; a RNA sequence encoding a heterologous protein that is operably linked to the gag 5′ UTR or a portion thereof; and a poly-A tail, wherein each of the R element of the 5′ LTR, the U5 element of the 5′ LTR, and the gag 5′ UTR or portion thereof is retroviral. In some embodiments, the RNA comprises a retroviral packaging sequence that is 3′ to the 5′ LTR.

Also provided herein is a lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a retroviral packaging sequence; a gag start codon; a RNA sequence encoding a heterologous protein; and a poly-A tail, wherein each of the R element of the 5′ LTR, the U5 element of the 5′ LTR, and the gag start codon is retroviral.

In some embodiments, the lipid particle further comprises a U3 element of a 5′ LTR. In some embodiments, the RNA comprises a polyadenylation site. In some embodiments, the RNA comprises a 3′ long terminal repeat (3′ LTR), and the polyadenylation site is located within a 3′ LTR. In some embodiments, the RNA comprises a mutated primer binding site (PBS). In some embodiments, the retroviral packaging sequence is selected from the group comprising HIV psi, MLV psi, SNV E, or a portion of any thereof. In some embodiments, the retroviral packaging sequence. HIV psi or a portion thereof. In some embodiments, the retroviral packaging sequence comprises stem-loop 1 (SL1), stem-loop 2 (SL2), stem-loop 3 (SL3), stem-loop 4 (SL4), or any combination thereof, of HIV psi. In some embodiments, the retroviral packaging sequence comprises stem-loop 1 (SL1) of HIV psi. In some embodiments, the retroviral packaging sequence comprises stem-loop 2 (SL2) of HIV psi. In some embodiments, the retroviral packaging sequence comprises stem-loop 3 (SL3) of HIV psi. In some embodiments, the retroviral packaging sequence comprises stem-loop 4 (SL4) of HIV psi. In some embodiments, the retroviral packaging sequence is HIV psi. In some embodiments, the RNA contained within the lipid particle comprises an HIV psi major splice donor site and is transcribed by the nucleic acid sequence set forth in SEQ ID NO: 205

In some embodiments, the retroviral packaging sequence is HIV psi. In some embodiments, the retroviral packaging sequence comprises a mutation in a major splice donor site. Without being limited to any theory, a mutation in a major splice donor site is expected to inhibit or prevent splicing at the major splice donor site, e.g., major splice donor site contained in SL2 of HIV psi, thereby causing all of the vector transcripts to be functional at full-length and packageable. In some embodiments, the major splice donor site is a major splice donor site contained in SL2 of HIV psi. In some embodiments, the mutation in the major splice donor site is a mutation, e.g., an inactivating mutation, that inhibits splicing at the major splice donor site. Accordingly, in some embodiments, the retroviral packaging sequence comprises a mutation in a major splice donor site of SL2 of HIV psi that inhibits splicing at the major splice donor site. In some embodiments, the RNA contained within the lipid particle comprises a mutated major splice donor site and is transcribed by the nucleic acid sequence set forth in SEQ ID NO: 204.

In some embodiments, the RNA comprises a retroviral sequence having at least about 80% sequence identity to the sequence of a retroviral genome that is about 20, 30, 40, 50, 60, 70, 80, 90, 100, 120, 140, 160, 180, 200, 220, 240, 260, 280, 300, 320, 340, 360, 380, or 400 nucleotides in length and comprises the gag start codon. In some embodiments, the RNA comprises a retroviral sequence having at least about 80% sequence identity to the sequence of a retroviral genome that is about 20, 30, 40, 50, 60, 70, 80, 90, 100, 120, 140, 160, 180, 200, 220, 240, 260, 280, 300, 320, 340, 360, 380, or 400 nucleotides in length and comprises a gag start codon. In some embodiments, the retroviral sequence comprises between about 20-400, between about 40 and about 350, between about 60 and about 300, between about 80 and about 250, or between about 100 and about 200 nucleotides 5′ to the gag start codon. In some embodiments, the retroviral sequence comprises between about 20 and about 400, between about 40 and about 350, between about 60 and about 300, between about 80 and about 250, or between about 100 and about 200 nucleotides 3′ to the gag start codon.

In some embodiments, the lumen comprises a capsid comprising a retroviral capsid protein enclosing the RNA. In some embodiments, the retroviral capsid protein and the retroviral packaging sequence are capable of associating with each other. In some embodiments, the retroviral capsid protein and the retroviral packaging sequence are from the same retroviral species. In some embodiments, the lipid particle comprises a retroviral matrix protein.

In some embodiments, the viral capsid protein comprises the amino acid residues of SEQ ID NO: 129. In some embodiments, the capsid protein comprises a sequence of amino acids that exhibits at least at or about 80%, 85%, 90% or 95% sequence identity to the sequence set forth in SEQ ID NO: 129. In some embodiments, the viral capsid protein comprises a cleavage site. In some embodiments, the viral capsid protein comprises the amino acid residues of SEQ ID NO: 130. In some embodiments, the viral capsid protein comprises a sequence of amino acids that exhibits at least at or about 80%, 85%, 90% or 95% sequence identity to the sequence set forth in SEQ ID NO:130.

In some embodiments, the lipid particle further comprises a RNA sequence encoding a viral structural protein or a portion thereof, which is located between the gag start codon and the RNA sequence encoding a heterologous protein. In some embodiments, the RNA does not comprise nucleotides between the gag start codon and the RNA sequence encoding a heterologous protein.

In some embodiments, wherein the RNA comprises a bicistronic element located between the RNA sequence encoding the viral structural protein or a portion thereof and the RNA sequence encoding the heterologous protein. In some embodiments, the bicistronic element is an internal ribosome entry site (IRES) element or a sequence encoding a 2A self-cleaving peptide. In some embodiments, the bicistronic element is a sequence encoding a 2A self-cleaving peptide. In some embodiments, the 2A self-cleaving peptide is T2A. In some embodiments, T2A comprises the sequence set forth in SEQ ID NO: 76. In some embodiments, the RNA encodes, from a 5′ to 3′ direction: the viral structural protein or portion thereof, T2A, and the heterologous protein.

In some embodiments, the viral structural protein is a retroviral gag. In some embodiments, the RNA sequence encoding the viral structural protein or a portion thereof encodes an N-terminal portion of a retroviral gag. In some embodiments, the RNA encodes a sequence of amino acids that exhibits at least at or about 80%, 85%, 90% or 95% sequence identity to the sequence set forth in SEQ ID NO:131. In some embodiments, the RNA encodes a sequence of amino acids set forth in SEQ ID NO: 131. In some embodiments, the RNA sequence encoding the viral structural protein or a portion thereof comprises the sequence set forth in SEQ ID NO:52. In some embodiments, the RNA encodes the sequence set forth in SEQ ID NO: 77 or 136 and the heterologous protein.

In some embodiments, the RNA is present as a first genomic viral RNA and the lipid particle further comprises a second genomic viral RNA. In some embodiments, the first genomic viral RNA and the second viral genomic RNA genome are identical. In some embodiments, the first genomic viral RNA and the second viral genomic RNA genome are different.

In some embodiments, the heterologous protein is a first heterologous protein and the lipid particle further comprises one or more additional heterologous protein(s), wherein the lipid particle comprises: (a) a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}; and/or (b) a fusion protein comprising a viral matrix (MA) protein and the one or more additional heterologous protein(s). In some embodiments, the heterologous protein is a first heterologous protein and the lipid particle further comprises one or more additional heterologous protein(s), wherein the lipid particle comprises a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}. In some embodiments, the heterologous protein is a first heterologous protein and the lipid particle further comprises one or more additional heterologous protein(s), wherein the lipid particle comprises a fusion protein comprising a viral matrix (MA) protein and the one or more additional heterologous protein(s). In some embodiments, the heterologous protein is a first heterologous protein and the lipid particle further comprises one or more additional heterologous protein(s), wherein the lipid particle comprises: (a) a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}; and (b) a fusion protein comprising a viral matrix (MA) protein and the one or more additional heterologous protein(s).

In some embodiments, the viral MA protein in (a) and/or (b) is derived from human immunodeficiency virus (HIV). In some embodiments, the viral MA protein in (a) is derived from human immunodeficiency virus (HIV). In some embodiments, the viral MA protein in (b) is derived from human immunodeficiency virus (HIV). In some embodiments, the viral MA protein in (a) and (b) is derived from human immunodeficiency virus (HIV). In some embodiments, the viral MA protein in (a) and/or (b) comprises the sequence set forth in SEQ ID NO:78. In some embodiments, the viral MA protein in (a) and/or (b) comprises the sequence set forth in SEQ ID NO: 127. In some embodiments, MS2_{cp} in (b) comprises the sequence set forth in SEQ ID NO:79. In some embodiments, the fusion protein of (a) comprises the sequence set forth in SEQ ID NO:74.

In some embodiments, the RNA comprises a 5′ cap. In some embodiments, the RNA is a self-inactivating lentiviral vector genome. In some embodiments, in a self-inactivating (SIN) vector, both LTR sequences may be modified to generate the self-inactivating vector. For instance, a SIN vector typically includes a deleted U3 (delU3) in which a large part of the U3 region is deleted, including portions containing the transcriptional enhancer and promoter. By deleting the transcriptional enhancers and/or the promoter in the U3 region of the LTR, the vector is replication limited so that following reverse transcription a full-length LTR cannot be reconstituted. In some aspects, SIN vectors have a deletion in the 3′-LTR covering the promoter/enhancer elements from the U3 region, e.g. about a 50 to about a 400 base pair deletion. In some embodiments, the SIN vector comprises a deleted U3 region, wherein said deletion includes a deletion of the TATA box. The deletion may be one that removes the TATA box, preventing transcription initiation and therefore inactivating the virus Miyoshi et al. 1998; Zuffrey et al 1998). In some aspects, this 3′-LTR deletion removes the polyadenylation signal distal to the TATA box. In some aspects, the 3′-LTR deletion removes the integrase recognition and processing site.

Also provided herein is a lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a RNA sequence encoding a viral structural protein or a portion thereof; a RNA sequence encoding a heterologous protein; and a poly-A tail, wherein each of the R element of the 5′ LTR and the U5 element of the 5′ LTR is retroviral. In some embodiments, the viral structural protein or a portion thereof is a retroviral structural protein or a portion thereof.

In some embodiments, the RNA comprises a bicistronic element located between the RNA sequence encoding the viral structural protein or a portion thereof and the RNA sequence encoding the heterologous protein. In some embodiments, the bicistronic element is an internal ribosome entry site (IRES) element or a sequence encoding a 2A self-cleaving peptide. In some embodiments, the bicistronic element is a sequence encoding a 2A self-cleaving peptide. In some embodiments, the 2A self-cleaving peptide is T2A. In some embodiments. T2A comprises the sequence set forth in SEQ ID NO:76. In some embodiments, the RNA encodes, from a 5′ to 3′ direction: the viral structural protein or portion thereof, T2A, and the heterologous protein.

In some embodiments, the viral structural protein is a retroviral gag. In some embodiments, the RNA sequence encoding the viral structural protein or a portion thereof encodes an N-terminal portion of a retroviral gag. In some embodiments, the RNA sequence encoding the viral structural protein or a portion thereof comprises the sequence set forth in SEQ ID NO:52. In some embodiments, the RNA encodes the sequence set forth in SEQ ID NO: 77 or 136 and the heterologous protein.

In some embodiments, the RNA is present as a first genomic viral RNA and the lipid particle further comprises a second genomic viral RNA. In some embodiments, the first genomic viral RNA and the second viral genomic RNA genome are identical. In some embodiments, the first genomic viral RNA and the second viral genomic RNA genome are different.

In some embodiments, the heterologous protein is a first heterologous protein and the lipid particle further comprises one or more additional heterologous protein(s). In some embodiments, the lipid particle comprises: (a) a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}; and/or (b) a fusion protein comprising a viral matrix (MA) protein and the one or more additional heterologous protein(s). In some embodiments, the lipid particle comprises a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}. In some embodiments, the lipid particle comprises a fusion protein comprising a viral matrix (MA) protein and the one or more additional heterologous protein(s). In some embodiments, the lipid particle comprises: (a) a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}; and (b) a fusion protein comprising a viral matrix (MA) protein and the one or more additional heterologous protein(s).

In some embodiments, the viral MA protein in (a) and/or (b) is derived from a retrovirus (e.g., human immunodeficiency virus (HIV)). In some embodiments, the viral MA protein in (a) and/or (b) is derived from human immunodeficiency virus (HIV). In some embodiments, the viral MA protein in (a) is derived from human immunodeficiency virus (HIV). In some embodiments, the viral MA protein in (b) is derived from human immunodeficiency virus (HIV). In some embodiments, the viral MA protein in (a) and (b) is derived from human immunodeficiency virus (HIV). In some embodiments, the viral MA protein in (a) and/or (b) comprises the sequence set forth in SEQ ID NO:78. In some embodiments, MS2_{cp} in (b) comprises the sequence set forth in SEQ ID NO:79. In some embodiments, the fusion protein of (a) comprises the sequence set forth in SEQ ID NO:74.

In some of any of such embodiments, the RNA sequence encoding a heterologous protein comprises a plurality of MS2_{cp}-binding loops. In some embodiments, the plurality of MS2_{cp}-binding loops comprises at or a at least 2, 5, 6, 10, 12, 15, 20, or 24 MS2_{cp}-binding loops. In some embodiments, the RNA sequence encoding a heterologous protein comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops. In some embodiments, the plurality of MS2_{cp}-binding loops comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops. In some embodiments, the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops. In some embodiments, the plurality of MS2_{cp}-binding loops comprises between or between about 6 and 24 MS2_{cp}-binding loops. In some embodiments, the plurality of MS2_{cp}-binding loops comprises between or between about 12 and 24 MS2_{cp}-binding loops. In some embodiments, the plurality of MS2_{cp}-binding loops comprises between or between about 6 and 30 MS2_{cp}-binding loops. In some embodiments, the plurality of MS2_{cp}-binding loops comprises between or between about 12 and 30 MS2_{cp}-binding loops. In some embodiments, the MS2_{cp}-binding loop comprises the RNA sequence transcribed from the nucleic acid set forth in SEQ ID NO: 185. In some embodiments, each of the MS2_{cp}-binding loops in the plurality of MS2_{cp}-binding loops comprises the RNA sequence transcribed from the nucleic acid set forth in SEQ ID NO: 185. In some embodiments, the MS2_{cp}-binding loop comprises the RNA sequence transcribed from the nucleic acid set forth in SEQ ID NO: 174. In some embodiments, each of the MS2_{cp}-binding loops in the plurality of MS2_{cp}-binding loops comprises the RNA sequence transcribed from the nucleic acid set forth in SEQ ID NO: 174. In some embodiments, the plurality of MS2_{cp}-binding loops comprises the RNA sequence transcribed from a nucleic acid sequence selected from the group consisting of SEQ ID NOs: 175-178. In some embodiments, the MS2_{cp}-binding loop comprises the RNA sequence set forth in SEQ ID NO: 208. In some embodiments, each of the MS2_{cp}-binding loops in the plurality of MS2_{cp}-binding loops comprises the RNA sequence set forth in SEQ ID NO: 208. In some embodiments, the MS2_{cp}-binding loop comprises the nucleic acid sequence set forth in: X₁X₂X₃X₄AXX₆AX₇PAX₈X₉X₁₀X₁₁X₁₂X₁₃ where X is any nucleotide, but the following pairs are complementary: X₁ and X₁₃, X₂ and X₁₂, X₃ and X₁₁, X₄ and X₁₀. X₅ and X₉, X₆ and X₈, and P is a pyrimidine.

In some embodiments, the RNA comprises a 5′ cap. In some embodiments, the RNA is a self-inactivating lentiviral vector genome. In some embodiments, in a self-inactivating (SIN) vector, both LTR sequences may be modified to generate the self-inactivating vector. For instance, a SIN vector typically includes a deleted U3 (delU3) in which a large part of the U3 region is deleted, including portions containing the transcriptional enhancer and promoter. By deleting the transcriptional enhancers and/or the promoter in the U3 region of the LTR, the vector is replication limited so that following reverse transcription a full-length LTR cannot be reconstituted. In some aspects, SIN vectors have a deletion in the 3′-LTR covering the promoter/enhancer elements from the U3 region, e.g. about a 50 to about a 400 base pair deletion. In some embodiments, the SIN vector comprises a deleted U3 region, wherein said deletion includes a deletion of the TATA box. The deletion may be one that removes the TATA box, preventing transcription initiation and therefore inactivating the virus Miyoshi et al. 1998; Zuffrey et al 1998). In some aspects, this 3′-LTR deletion removes the polyadenylation signal distal to the TATA box. In some aspects, the 3′-LTR deletion removes the integrase recognition and processing site.

In some embodiments, the viral RNA fusion does not encode reverse transcriptase.

In some embodiments, the RNA comprises a mutated primer binding site (PBS). In some aspects, a mutation in the PBS prevents a primer from initiating reverse transcription. Methods of mutating a PBS to prevent reverse transcription are known in the art and include any of those as described in Aiyar et al., J. Virol. (1994) 68 (2): 611-18; Li et al., J. Virol. (1994) 68 (10): 6198-6206; Lund et al., J. Virol. (1997) 71 (2): 1191-95; Rhim et al., J. Virol. (1991) 65 (0): 4555-64; and Wakefield et al., J. Virol. (1994) 68 (3): 1605-14, each of which is incorporated herein in its entirety.

In some embodiments, the heterologous protein is a genome-modifying protein. In some embodiments, the genome-modifying protein comprises a recombinant nuclease, a nickase, an integrase, reverse transcriptase, or a combination thereof. In some embodiments, the genome-modifying protein comprises a zinc-finger nuclease (ZFN), a transcription-activator like effector nucleases (TALEN), or a CRISPR-associated (Cas) protein. In some embodiments, the genome-modifying protein is a Cas protein. In some embodiments, the genome-modifying protein is Cas9. In some embodiments, the genome-modifying protein is saCas9. In some embodiments, the genome-modifying protein is spCas9. In some embodiments, the genome-modifying protein is cpf1. In some embodiments, a Cas protein comprises a core Cas protein. Exemplary Cas core proteins include, but are not limited to, Cas1, Cas2, Cas3, Cas4, Cas5, Cas6, Cas7, Cas8, Cas9, Cas12a (also known as Cpf1), Cas12b, Cas1212, Cas13, and Mad7. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1. Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. Sec, e.g., Klompe et al., Nature 571, 219-225 (2019); Strecker et al., Science 365, 48-53 (2019).

In some embodiments, the lipid particle comprises a guide RNA (gRNA) in the lumen. In some embodiments, the gRNA is a single guide RNA (sgRNA).

In some embodiments, the lipid particle is pseudotyped with a viral envelope glycoprotein. In some embodiments, the viral envelope glycoprotein is a VSV-G protein or a functional variant thereof. In some embodiments, the viral envelope glycoprotein is a Cocal virus G protein or a functional variant thereof. In some embodiments, the viral envelope glycoprotein is an Alphavirus fusion protein (e.g. Sindbis virus) or a functional variant thereof. In some embodiments, the viral envelope glycoprotein is a Paramyxoviridae fusion protein (e.g., a Morbillivirus or a Henipavirus) or a functional variant thereof. In some embodiments, the viral envelope glycoprotein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof. In some embodiments, the viral envelope glycoprotein is a Henipavirus fusion protein (e.g., Nipah virus, Hendra virus, Cedar virus, Kumasi virus, Mòjiāng virus, Langya virus) or a functional variant thereof.

In some embodiments, the viral envelope glycoprotein comprises one or more modifications to reduce binding to its native receptor. In some embodiments, the viral envelope glycoprotein comprises a Nipah virus F glycoprotein (NiV-F) or a biologically active portion thereof and a Nipah virus G glycoprotein (NiV-G) or a biologically active portion thereof. In some embodiments, the NiV-G or the biologically active portion thereof is a wild-type NiV-G protein or a functionally active variant or biologically active portion thereof.

In some embodiments, the Niv-F or a biologically active portion thereof comprises the amino acid sequence set forth in SEQ ID NO: 145, or an amino acid sequence having at least 90%, 91%, 92%, 93%, 94%, 95%, 96%, 97%, 98%, or 99% sequence identity to the amino acid sequence set forth in SEQ ID NO: 145; and/or the Niv-G or a biologically active portion thereof comprises the amino acid sequence set forth in SEQ ID NO: 147, or an amino acid sequence having at least 90%, 91%, 92%, 93%, 94%, 95%, 96%, 97%, 98%, or 99% sequence identity to the amino acid sequence set forth in SEQ ID NO: 147. In some embodiments, the Niv-F or a biologically active portion thereof comprises the amino acid sequence set forth in SEQ ID NO: 145, or an amino acid sequence having at least 90%, 91%, 92%, 93%, 94%, 95%, 96%, 97%, 98%, or 99% sequence identity to the amino acid sequence set forth in SEQ ID NO: 145; and the Niv-G or a biologically active portion thereof comprises the amino acid sequence set forth in SEQ ID NO: 147, or an amino acid sequence having at least 90%, 91%, 92%, 93%, 94%, 95%, 96%, 97%, 98%, or 99% sequence identity to the amino acid sequence set forth in SEQ ID NO: 147. In some embodiments, the Niv-F or a biologically active portion thereof comprises the amino acid sequence set forth in SEQ ID NO: 145; and/or the Niv-G or a biologically active portion thereof comprises the amino acid sequence set forth in SEQ ID NO: 147. In some embodiments, the Niv-F or a biologically active portion thereof comprises the amino acid sequence set forth in SEQ ID NO: 145; and the Niv-G or a biologically active portion thereof comprises the amino acid sequence set forth in SEQ ID NO: 147.

In some embodiments, the NiV-G protein or the biologically active portion is truncated and lacks up to 40 contiguous amino acid residues at or near the N-terminus of the wild-type NiV-G protein. In some embodiments, the NiV-G protein or the biologically active portion has a 5 amino acid truncation at or near the N-terminus of the wild-type NiV-G protein, optionally wherein the NiV-G protein or the biologically active portion thereof has the amino acid sequence set forth in SEQ ID NO: 12, or a sequence of amino acids that exhibits at least at or about 80%, 85%, 90% or 95% sequence identity to the sequence set forth in SEQ ID NO: 12. In some embodiments, the NiV-G protein or the biologically active portion has a 10 amino acid truncation at or near the N-terminus of the wild-type NiV-G protein, optionally wherein the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44. In some embodiments, the NiV-G protein or the biologically active portion has a 15 amino acid truncation at or near the N-terminus of the wild-type NiV-G protein, optionally wherein the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45. In some embodiments, the NiV-G protein or the biologically active portion has a 20 amino acid truncation at or near the N-terminus of the wild-type NiV-G protein, optionally wherein the NiV-G protein or the biologically active portion thereof has the amino acid sequence set forth in SEQ ID NO: 13, or a sequence of amino acids that exhibits at least at or about 80%, 85%, 90% or 95% sequence identity to the sequence set forth in SEQ ID NO:13. In some embodiments, the NiV-G protein or the biologically active portion has a 25 amino acid truncation at or near the N-terminus of the wild-type NiV-G protein, optionally wherein the NiV-G protein or the biologically active portion thereof has the amino acid sequence set forth in SEQ ID NO: 14, or a sequence of amino acids that exhibits at least at or about 80%, 85%, 90% or 95% sequence identity to the sequence set forth in SEQ ID NO: 14. In some embodiments, the NiV-G protein or the biologically active portion has a 30 amino acid truncation at or near the N-terminus of the wild-type NiV-G protein, optionally wherein the NiV-G protein or the biologically active portion thereof has the amino acid sequence set forth in SEQ ID NO: 43, or a sequence of amino acids that exhibits at least at or about 80%, 85%, 90% or 95% sequence identity to the sequence set forth in SEQ ID NO:43. In some embodiments, the NiV-G protein or the biologically active portion has a 34 amino acid truncation at or near the N-terminus of the wild-type NiV-G protein, optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.

In some embodiments, the NiV-G-protein or the biologically active portion thereof is a mutant NiV-G protein that exhibits reduced binding to Ephrin B2 or Ephrin B3. In some embodiments, the mutant NiV-G protein or the biologically active portion comprises one or more amino acid substitutions corresponding to amino acid substitutions selected from the group consisting of E501A, W504A, Q530A and E533A with reference to numbering set forth in SEQ ID NO: 4.

In some embodiments, the mutant NiV-G protein or the biologically active portion comprises the amino acid sequence set forth in SEQ ID NO: 17 or a sequence of amino acids that exhibits at least at or about 80%, 85%, 90% or 95% sequence identity to the sequence set forth in SEQ ID NO: 17. In some embodiments, the NiV-G protein or the biologically active portion has the amino acid sequence set forth in SEQ ID NO: 18 or a sequence of amino acids that exhibits at least at or about 80%, 85%, 90% or 95% sequence identity to the sequence set forth in SEQ ID NO: 18. In some embodiments, the NiV-F protein or the biologically active portion thereof is a wild-type NiV-F protein or is a functionally active variant or a biologically active portion thereof. In some embodiments, the NiV-F protein or the biologically active portion thereof has a 20 amino acid truncation at or near the C-terminus of the wild-type NiV-F protein, optionally wherein the NiV-F protein or the biologically active portion thereof comprises the sequence set forth in SEQ ID NO: 20 or a sequence of amino acids that exhibits at least at or about 80%, 85%, 90% or 95% sequence identity to the sequence set forth in SEQ ID NO: 20.

In some embodiments, the NiV-F protein or the biologically active portion thereof comprises: i) a 20 amino acid truncation at or near the C-terminus of the wild-type NiV-F protein; and ii) a point mutation on an N-linked glycosylation site. In some embodiments, the NiV-F protein or the biologically active portion thereof comprises the sequence set forth in SEQ ID NO: 15, or a sequence of amino acids that exhibits at least at or about 80%, 85%, 90% or 95% sequence identity to the sequence set forth in SEQ ID NO: 15. In some embodiments, the NiV-F protein or the biologically active portion thereof has a 22 amino acid truncation at or near the C-terminus of the wild-type NiV-F protein. In some embodiments, the NiV-F protein or the biologically active portion thereof comprises the sequence set forth in SEQ ID NO: 16, 19, or 21 or a sequence of amino acids that exhibits at least at or about 80%, 85%, 90% or 95% sequence identity to the sequence set forth in SEQ ID NO: 16, 19, or 21.

In some embodiments, the NiV-F protein or the biologically active portion thereof comprises the amino acid sequence set forth in SEQ ID NO:21, or a sequence of amino acids that exhibits at least at or about 80%, 85%, 90% or 95% sequence identity to the sequence set forth in SEQ ID NO:21. In some embodiments, the Niv-G protein comprises the amino acid sequence set forth in SEQ ID NO: 17, and the Niv-F protein comprises the amino acid sequence set forth in SEQ ID NO: 21.

In some embodiments, the lipid particle comprises a targeting moiety. In some embodiments, the targeting moiety binds to a target cell. In some embodiments, the targeting moiety is a single domain antibody (sdAb). In some embodiments, the sdAb can be human or humanized. In some embodiments, the sdAb is a VHH. In some embodiments, the targeting moiety is a single chain molecule. In some embodiments, the targeting moiety is a single chain variable fragment (scFv). In particular embodiments, the targeting moiety contains an antibody variable sequence(s) that is human or humanized.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cadiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a I T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the targeting moiety binds to any one of CD3, CD8, CD4, asialoglycoprotein receptor 2 (ASGR2), transmembrane 4 L6 family member 5 (TM4SF5), low density lipoprotein receptor (LDLR) or asialoglycoprotein 1 (ASGR1).

In some embodiments, the targeting moiety is selected from the group consisting of a CD3-binding agent, a CD8-binding agent, and a CD4-binding agent. In some embodiments, the targeting moiety is a CD3-binding agent, optionally an anti-CD3 antibody or an antigen-binding fragment.

In some embodiments, the targeting moiety is a CD8-binding agent, optionally an anti-CD8 antibody or an antigen-binding fragment. In some embodiments, the targeting moiety is a CD4-binding agent, optionally an anti-CD4 antibody or an antigen-binding fragment. In some embodiments, the targeting moiety is exposed on the surface of the lipid particle. In some embodiments, the targeting moiety is fused to a transmembrane domain incorporated into the bilayer of the lipid particle.

In some embodiments, the lipid particle is a retroviral vector or a retroviral-like particle. In some embodiments, the retroviral vector or the retroviral-like particle is replication-deficient. In some embodiments, the lipid particle does not comprise reverse transcriptase or does not comprise reverse transcriptase activity. In some embodiments, the lipid particle does not comprise a protein with reverse transcriptase activity. In some embodiments, the lipid particle does not comprise reverse transcriptase. In some embodiments, the lipid particle comprises non-functional reverse transcriptase. In some embodiments, the reverse transcriptase is mutated. In some embodiments, the retroviral vector or retroviral-like particle comprises a RNA that is a self-inactivating lentiviral vector genome. In some embodiments, the retroviral vector or retroviral-like particle comprises a RNA comprising a 3′LTR, and the 3′ LTR does not comprise a functional U3 domain. In some embodiments, the U3 domain comprises a deletion.

In some embodiments, the lipid particle is a retroviral particle, and the retroviral particle is a lentiviral particle. In some embodiments, the lipid particle is a retrovirus-like particle (VLP).

In some embodiments, the lipid bilayer is derived from a host cell. In some embodiments, the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

Also provided herein is a method of producing a lipid particle comprising a lipid bilayer enclosing a lumen and a viral ribonucleic acid. In some embodiments, the method comprises: (1) providing a host cell comprising (a) a nucleic acid sequence selected from the group consisting of: a 5′ long terminal repeat (5′ LTR); a psi packaging signal sequence; a gag start codon; a RNA sequence encoding a heterologous protein; a 3′ long terminal repeat (3′ LTR); or a combination thereof; and (b) a nucleic acid sequence encoding a protein selected from the group consisting of gag, pol, rev, tat, a viral envelope glycoprotein, or a combination thereof; and (2) culturing the host cell under conditions to induce packaging of the lipid particle. In some embodiments, the lipid particle comprises a RNA sequence encoding a viral structural protein or a portion thereof, which is located between the gag start codon and the RNA sequence encoding a heterologous protein. In some embodiments, the gag start codon and the RNA sequence encoding a heterologous protein are part of the same RNA, and the RNA does not comprise nucleotides between the gag start codon and the RNA sequence encoding a heterologous protein. In some embodiments, a bicistronic element is located between the RNA sequence encoding the viral structural protein or portion thereof and the RNA sequence encoding the heterologous protein. The bicistronic element is an internal ribosome entry site (IRES) element or a sequence encoding a 2A self-cleaving peptide. In some embodiments, the bicistronic element is a sequence encoding a 2A self-cleaving peptide. In some embodiments, the 2A self-cleaving peptide is T2A. In some embodiments, the sequence set forth in SEQ ID NO:76. In some embodiments, the RNA sequence encodes, from a 5′ to 3′ direction: the viral structural protein or portion thereof, T2A, and the heterologous protein.

In some embodiments, the viral structural protein is gag. In some embodiments, the RNA sequence encoding the viral structural protein or a portion thereof encodes an N-terminal portion of gag. In some embodiments, the RNA sequence encoding the viral structural protein or a portion thereof comprises the sequence set forth in SEQ ID NO:52. In some embodiments, the host cell comprises a nucleic acid sequence that comprises the sequence set forth in SEQ ID NO:77, or 136 and encodes the heterologous protein.

In some embodiments, the viral envelope glycoprotein is VSV-G.

In some embodiments, the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

In some embodiments, the nucleic acid sequence in (b) comprises a 5′ promoter. In some embodiments, the promoter is a cytomegalovirus (CMV) promoter.

Also provided herein is a lipid particle produced by any of the methods provided herein. Also provided herein is a composition comprising any of the lipid particles described herein.

Also provided herein is a method of introducing a heterologous protein into a target cell, the method comprising contacting the target cell with a lipid particle or composition provided herein. Also provided herein is a method of genetically engineering a target cell, the method comprising contacting the target cell with a lipid particle or composition provided herein. In some embodiments, the contacting is in vitro or ex vivo. In some embodiments, the contacting is in vivo.

Also provide herein is a deoxyribonucleic acid (DNA) sequence encoding a gag start codon and a heterologous protein. In some embodiments, the DNA sequence encodes a viral structural protein or a portion thereof, wherein the portion of the DNA sequence encoding the viral structural protein is located between the portions of the DNA sequence encoding the gag start codon and the heterologous protein. In some embodiments, the DNA sequence encodes a bicistronic element, wherein the portion of the DNA sequence encoding the bicistronic element is located between the portions of the DNA sequence encoding the viral structural protein or a portion thereof and the heterologous protein. In some embodiments, the bicistronic element is an internal ribosome entry site (IRES) element or a sequence encoding a 2A self-cleaving peptide. In some embodiments, the 2A self-cleaving peptide is T2A. In some embodiments, T2A comprises the sequence set forth in SEQ ID NO:76. In some embodiments, the DNA sequence encodes from a 5′ to 3′ direction: the viral structural protein or portion thereof, T2A, and the heterologous protein.

In some embodiments, the viral structural protein is gag. In some embodiments, the DNA sequence encodes an N-terminal portion of gag. In some embodiments, the N-terminal portion of gag comprises the sequence set forth in SEQ ID NO:52. In some embodiments, the DNA sequence encodes the sequence set forth in SEQ ID NO:77, or 136 and the heterologous protein.

In some embodiments, the DNA sequence does not comprise nucleotides between the encoded gag start codon and the encoded heterologous protein. In some embodiments, the DNA sequence comprises a promoter. In some embodiments, the promoter is a cytomegalovirus (CMV) promoter.

In some embodiments, the heterologous protein is a genome-modifying protein. In some embodiments, the genome-modifying protein comprises a recombinant nuclease, a nickase, an integrase, reverse transcriptase, or a combination thereof. In some embodiments, the genome-modifying protein comprises a zinc-finger nuclease (ZFN), a transcription-activator like effector nucleases (TALEN), or a CRISPR-associated (Cas) protein. In some embodiments, the genome-modifying protein is a Cas protein. In some embodiments, the genome-modifying protein is (i) Cas9, optionally saCas9 or spCas9; or (ii) cpf1. In some embodiments, a Cas protein comprises a core Cas protein. Exemplary Cas core proteins include, but are not limited to, Cas1, Cas2, Cas3, Cas4, Cas5, Cas6, Cas7, Cas8, Cas9, Cas12a (also known as Cpf1), Cas12b, Cas1212, Cas13, and Mad7. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (019); Strecker et al., Science 365, 48-53 (2019).

In some embodiments, the heterologous protein is a tumor neoepitope. In some embodiments, the heterologous protein is a viral Spike(s) glycoprotein. In some embodiments, the heterologous protein is a protein from Zika virus, optionally Zika virus prM-E protein; tuberculosis; respiratory syncytial virus (RSV), optionally RSV fusion (RSV-F) protein; influenza virus, optionally influenza virus hemagglutinin (HA); rabies virus, optionally rabies virus glycoprotein (RABV-G); human cytolomegalovirus (CMV); hepatitis C virus; human immunodeficiency virus 1 (HIV-1), and Streptococcus. In some embodiments, the heterologous protein is an antibody or an antigen-binding fragment thereof.

Also provided herein is a vector comprising a DNA sequence provided herein. Also provided herein is a mammalian cell comprising a DNA sequence or vector provided herein. In some embodiments, the mammalian cell comprises a viral nucleic acid, wherein the viral nucleic acid lacks one or more genes involved in viral replication. In some embodiments, the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3); a nucleic acid encoding a viral envelope protein; and/or a nucleic acid encoding a viral packaging protein selected from one or more of gag, pol, rev and tat. In some embodiments, the mammaial cell comprises a RNA sequence encoding a heterologous protein. In some embodiments, the mammaial cell comprises a guide RNA (gRNA).

Also provide herein is a transfer plasmid comprising a promoter operably linked to a RNA sequence encoding a gag protein or portion thereof comprising at least a gag start codon; a RNA sequence encoding a heterologous protein that is linked to the RNA sequence encoding a gag protein or portion thereof; and a 3′ long terminal repeat (3′ LTR).

Also provided herein is a transfer plasmid comprising a promoter operably linked to a ribonucleic acid (RNA), wherein the RNA comprises, from 5′ to 3′: a 5′ long terminal repeat (5′ LTR); a gag 5′ untranslated region (UTR) or portion thereof comprising at least three nucleotides; a RNA sequence encoding a heterologous protein that is linked to the gag 5′ UTR or a portion thereof; and a 3′ long terminal repeat (3′ LTR).

Also provided herein is a transfer plasmid comprising a promoter operably linked to a ribonucleic acid (RNA), wherein the RNA comprises, from 5′ to 3′: a 5′ long terminal repeat (5′ LTR); a retroviral packaging sequence; a gag start codon; a RNA sequence encoding a heterologous protein; and a 3′ long terminal repeat (3′ LTR). In some embodiments, the transfer plasmid is a lentiviral transfer plasmid.

In some embodiments, sequences disclosed herein are expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

### 2. Tethering of RNA

Provided herein is a lipid particle comprising a lipid bilayer enclosing a lumen; a viral matrix (MA) protein; an RNA-binding protein; and an RNA sequence encoding a heterologous protein, wherein the RNA-binding protein is incorporated into the lipid particle as a fusion protein with the viral MA protein, and wherein the RNA sequence encoding the heterologous protein comprises a binding site for binding to the RNA-binding protein. In some embodiments, the RNA-binding protein is a MS2 coat protein (MS2_{cp}). In some embodiments, the RNA-binding protein is lambda N protein (λN) or a functional variant thereof. Accordingly, in some embodiments, provided herein is a lipid particle comprising a lipid bilayer enclosing a lumen; a viral matrix (MA) protein; a MS2 coat protein (MS2_{cp}); and an RNA sequence encoding a heterologous protein, wherein the MS2 coat protein (MS2_{cp}) is incorporated into the lipid particle as a fusion protein with the viral MA protein, and wherein the RNA sequence encoding the heterologous protein comprises a binding site for binding to the MS2 coat protein (MS2_{cp}). Also, in some embodiments, provided herein is a lipid particle comprising a lipid bilayer enclosing a lumen; a viral matrix (MA) protein; lambda N protein (λN) or a functional variant thereof; and an RNA sequence encoding a heterologous protein, wherein the lambda N protein (λN) or a functional variant thereof is incorporated into the lipid particle as a fusion protein with the viral MA protein, and wherein the RNA sequence encoding the heterologous protein comprises a binding site for binding to the lambda N protein (λN) or a functional variant thereof.

Also provided herein is a lipid particle comprising a lipid bilayer enclosing a lumen; a fusion protein comprising a viral envelope glycoprotein and an RNA-binding protein; and an RNA sequence encoding a heterologous protein, wherein the RNA sequence encoding the heterologous protein comprises a binding site for binding to the RNA-binding protein. In some embodiments, the viral envelope glycoprotein is VSV-G or a functional variant thereof. In some embodiments, the RNA-binding protein is a MS2 coat protein (MS2_{cp}). In some embodiments, the RNA-binding protein is lambda N protein (2N) or a functional variant thereof. Accordingly, in some embodiments, provided herein is a lipid particle comprising a lipid bilayer enclosing a lumen; a fusion protein comprising a VSV-G or a functional variant thereof and an RNA-binding protein; and an RNA sequence encoding a heterologous protein, wherein the RNA sequence encoding the heterologous protein comprises a binding site for binding to the RNA-binding protein. Also, in some embodiments, provided herein is a lipid particle comprising a lipid bilayer enclosing a lumen; a fusion protein comprising a VSV-G or a functional variant thereof and a MS2 coat protein (MS2_{cp}); and an RNA sequence encoding a heterologous protein, wherein the RNA sequence encoding the heterologous protein comprises a binding site for binding to the MS2 coat protein (MS2_{cp}). Also, in some embodiments, provided herein is a lipid particle comprising a lipid bilayer enclosing a lumen; a fusion protein comprising a VSV-G or a functional variant thereof and a lambda N protein (λN) or a functional variant thereof; and an RNA sequence encoding a heterologous protein, wherein the RNA sequence encoding the heterologous protein comprises a binding site for binding to the lambda N protein (2N) or a functional variant thereof.

Also provided herein is a lipid particle comprising a lipid bilayer enclosing a lumen; a fusion protein comprising a viral matrix (MA) protein and a MS2 coat protein (MS2_{cp}); and an RNA sequence encoding a heterologous protein. In some embodiments, the RNA sequence encoding a heterologous protein comprises a MS2_{cp}-binding loop In some embodiments, the RNA sequence comprising a MS2_{cp}-binding loop for which a heterologous protein may be inserted is transcribed from the nucleic acid set forth in SEQ ID NO: 207.

Also provided herein is a lipid bilayer enclosing a lumen; a viral matrix (MA) protein; a MS2 coat protein (MS2_{cp}); and an RNA sequence encoding a heterologous protein, wherein the MS2_{cp} is incorporated into the lipid particle as a fusion protein with the viral MA protein, and wherein the RNA sequence encoding the heterologous protein comprises a MS2_{cp}-binding loop for binding to MS2_{cp}.

In some embodiments, the fusion protein dissociates within the lipid particle, such that the viral MA protein and MS2_{cp} are present in the lipid particle as separate polypeptides. In some embodiments, the fusion protein comprising the viral MA protein and MS2_{cp} is cleaved, such that the viral MA protein and MS2_{cp} are present in the lipid particle as separate polypeptides. In some embodiments, the fusion particle is cleaved at a cleavage sequence. In some embodiments, the cleavage sequence is located between the viral MA protein and MS2_{cp}. In some embodiments, the cleavage sequence comprises the amino acid residues PIVQ (SEQ ID: 140). In some embodiments, the cleavage sequence is the amino acid residues PIVQ (SEQ ID NO:140). In some embodiments, the cleavage sequence comprises the sequence set forth in SEQ ID NO: 128. In some embodiments, the cleavage sequence is the sequence set forth in SEQ ID NO: 128. In some embodiments, the cleavage sequence comprises the amino acid residues SQNYPIVQ (SEQ ID: 128). In some embodiments, the cleavage sequence is the amino acid residues SQNYPIVQ (SEQ ID NO: 128). In some embodiments, the MA protein comprises a sequence of amino acids that exhibits at least at or about 80%, 85%, 90% or 95% sequence identity to the sequence set forth in SEQ ID NO: 137 or 139. In some embodiments, the MA protein comprises a sequence of amino acids set forth in SEQ ID NO: 137 or 139. In some embodiments, the MS2_{cp} protein comprises a sequence of amino acids that exhibits at least at or about 80%, 85%, 90% or 95% sequence identity to the sequence set forth in SEQ ID NO: 138. In some embodiments, the MS2_{cp} protein comprises a sequence of amino acids set forth in SEQ ID NO: 138.

In some embodiments, the fusion protein further comprises a viral capsid (CA) protein. Thus, in some embodiments, the fusion protein comprises, from a 5′ to 3′ direction: the viral MA protein, MS2_{cp}, and the viral CA protein. In some embodiments, the viral capsid protein comprises the amino acid residues of SEQ ID NO: 129. In some embodiments, the capsid protein comprises a sequence of amino acids that exhibits at least at or about 80%, 85%, 90% or 95% sequence identity to the sequence set forth in SEQ ID NO: 129. In some embodiments, the viral capsid protein comprises a cleavage site. In some embodiments, the viral capsid protein comprises the amino acid residues of SEQ ID NO: 130. In some embodiments, the viral capsid protein comprises a sequence of amino acids that exhibits at least at or about 80%, 85%, 90% or 95% sequence identity to the sequence set forth in SEQ ID NO: 130.

In some embodiments, the fusion protein dissociates within the lipid particle, such that the viral MA protein, MS2_{cp}, and the viral CA protein are each present in the lipid particle as separate polypeptides. In some embodiments, the fusion protein comprising the viral MA protein, MS2_{cp}, and the viral CA protein is cleaved, such that each of the viral MA protein, MS2_{cp}, and the viral CA protein are present in the lipid particle as separate polypeptides. In some embodiments, the fusion particle is cleaved at a cleavage sequence. In some embodiments, the cleavage sequence is located between the viral MA protein and MS2_{cp}. In some embodiments, the cleavage sequence is located between MS2_{cp} and the viral CA protein. In some embodiments, the cleavage sequence is located between the viral MA protein and MS2_{cp} and between MS2_{cp} and the viral CA protein. Thus, in some embodiments, the fusion protein comprises two cleavage sequences. In some embodiments, the cleavage sequence comprises the amino acid residues PIVQ (SEQ ID NO: 140). In some embodiments, the cleavage sequence is the amino acid residues PIVQ (SEQ ID NO:140). In some embodiments, the cleavage sequence comprises the sequence set forth in SEQ ID NO: 128. In some embodiments, the cleavage sequence is the sequence set forth in SEQ ID NO:128. In some embodiments, the cleavage sequence comprises the amino acid residues SQNYPIVQ (SEQ ID NO:128). In some embodiments, the cleavage sequence is the amino acid residues SQNYPIVQ (SEQ ID NO:128).

In some embodiments, the viral MA protein reversibly binds to the lipid bilayer. In some embodiments, the fusion protein comprises, from a 5′ to 3′ direction: the viral MA protein and MS2_{cp}. In some embodiments, the RNA sequence encoding a heterologous protein comprises a MS2_{cp}-binding loop.

In some embodiments, the RNA sequence encoding a heterologous protein comprises 12 or 24 MS2_{cp}-binding loops. In some embodiments, the RNA sequence encoding a heterologous protein comprises 12 MS2_{cp}-binding loops. In some embodiments, the RNA sequence encoding a heterologous protein comprises 24 MS2_{cp}-binding loops. In some embodiments, the viral MA protein is attached to a portion of the lipid bilayer that is in contact with the lumen. In some embodiments, the viral MA protein reversibly binds to the lipid bilayer. Thus, in some embodiments, the RNA sequence encoding the heterologous protein is tethered to MS2_{cp} by binding to the loops of MS2_{cp}, and MS2_{cp} is fused with the viral MA protein, which is attached to or integrated with a portion of the lipid bilayer. Thus, in some aspects, the fusion protein tethers the RNA sequence encoding the heterologous protein to the lipid bilayer.

In some embodiments, the fusion protein dissociates within the lipid particle, such that the viral MA protein. λN, and the viral CA protein are each present in the lipid particle as separate polypeptides. In some embodiments, the fusion protein comprising the viral MA protein, λN, and the viral CA protein is cleaved, such that each of the viral MA protein, λN, and the viral CA protein are present in the lipid particle as separate polypeptides. In some embodiments, the fusion particle is cleaved at a cleavage sequence. In some embodiments, the cleavage sequence is located between the viral MA protein and λN. In some embodiments, the cleavage sequence is located between λN and the viral CA protein. In some embodiments, the cleavage sequence is located between the viral MA protein and λN and between λN and the viral CA protein. Thus, in some embodiments, the fusion protein comprises two cleavage sequences. In some embodiments, the cleavage sequence comprises the amino acid residues PIVQ (SEQ ID NO:140). In some embodiments, the cleavage sequence is the amino acid residues PIVQ (SEQ ID NO:140). In some embodiments, the cleavage sequence comprises the sequence set forth in SEQ ID NO:128. In some embodiments, the cleavage sequence is the sequence set forth in SEQ ID NO: 128. In some embodiments, the cleavage sequence comprises the amino acid residues SQNYPIVQ (SEQ ID NO:128). In some embodiments, the cleavage sequence is the amino acid residues SQNYPIVQ (SEQ ID NO:128).

In some embodiments, the viral MA protein reversibly binds to the lipid bilayer. In some embodiments, the fusion protein comprises, from a 5′ to 3′ direction: the viral MA protein and λN. In some embodiments, the RNA sequence encoding a heterologous protein comprises a boxB binding site for binding to λN. In some embodiments, the RNA sequence encoding a heterologous protein comprises 12 or 24 boxB binding sites. In some embodiments, the RNA sequence encoding a heterologous protein comprises 12 boxB binding sites. In some embodiments, the RNA sequence encoding a heterologous protein comprises 24 boxB binding sites. In some embodiments, the viral MA protein is attached to a portion of the lipid bilayer that is in contact with the lumen. In some embodiments, the viral MA protein reversibly binds to the lipid bilayer. Thus, in some embodiments, the RNA sequence encoding the heterologous protein is tethered to λN by binding to the loops of λN, and λN is fused with the viral MA protein, which is attached to or integrated with a portion of the lipid bilayer. Thus, in some aspects, the fusion protein tethers the RNA sequence encoding the heterologous protein to the lipid bilayer.

In some embodiments, the viral MA protein is derived from human immunodeficiency virus (HIV). In some embodiments, the viral MA protein comprises the sequence set forth in SEQ ID NO:78. In some embodiments, MS2_{cp} comprises the sequence set forth in SEQ ID NO:79. In some embodiments, the fusion protein comprises the sequence set forth in SEQ ID NO:74. In some embodiments, the fusion protein comprises the sequence set forth in SEQ ID NO:191.

In some embodiments, the lipid particle comprises a transfer plasmid encoding a guide RNA (gRNA). In some embodiments, the gRNA is a single guide RNA (sgRNA). In some embodiments, the gRNA is under the control of a U6 promoter.

In some embodiments, the lipid particle comprises a lipid bilayer enclosing a lumen; a fusion protein comprising a viral envelope glycoprotein and an RNA-binding protein; and an RNA sequence encoding a heterologous protein. In some embodiments, the fusion protein comprises, from an N-terminus to C-terminus direction: the viral envelope glycoprotein and the RNA binding protein, wherein the RNA sequence encoding the heterologous protein comprises a binding site for binding to the RNA-binding protein. In some embodiments, the RNA-binding protein is fused to the C-terminus of the viral envelope glycoprotein.

In some embodiments, the binding site for binding to the RNA-binding protein is contained in the 3′ UTR of the RNA sequence encoding the heterologous protein.

In some embodiments, the lipid particle comprises a lipid bilayer enclosing a lumen; a fusion protein comprising a VSV-G protein or a functional variant thereof and an RNA-binding protein; and an RNA sequence encoding a heterologous protein, wherein the RNA sequence encoding the heterologous protein comprises a binding site for binding to the RNA-binding protein.

In some embodiments, the VSV-G or a functional variant thereof comprises the amino acid sequence of SEQ ID NO: 199, or an amino acid sequence having at least 90%, 91%, 92%, 93%, 94%, 95%, 96%, 97%, 98%, or 99% sequence identity to the amino acid sequence of SEQ ID NO: 199. In some embodiments, the VSV-G or a functional variant thereof comprises the amino acid sequence of SEQ ID NO: 199. In some embodiments, the fusion protein comprises, from an N-terminus to C-terminus direction: the VSV-G protein or a functional variant thereof and the RNA binding protein. In some embodiments, the RNA-binding protein is fused directly or indirectly to the C-terminus of the VSV-G protein or a functional variant thereof.

In some embodiments, the heterologous protein is a first heterologous protein and the lipid particle further comprises one or more additional heterologous protein(s), wherein the lipid particle comprises: (a) a RNA comprising a RNA sequence encoding the one or more additional heterologous protein(s) and a RNA sequence encoding a viral structural protein or a portion thereof; and/or (b) a fusion protein comprising a viral matrix (MA) protein and the one or more additional heterologous protein(s). In some embodiments, the heterologous protein is a first heterologous protein and the lipid particle further comprises one or more additional heterologous protein(s), wherein the lipid particle comprises a RNA comprising a RNA sequence encoding the one or more additional heterologous protein(s) and a RNA sequence encoding a viral structural protein or a portion thereof. In some embodiments, the heterologous protein is a first heterologous protein and the lipid particle further comprises one or more additional heterologous protein(s), wherein the lipid particle comprises a fusion protein comprising a viral matrix (MA) protein and the one or more additional heterologous protein(s). In some embodiments, the heterologous protein is a first heterologous protein and the lipid particle further comprises one or more additional heterologous protein(s), wherein the lipid particle comprises: (a) a RNA comprising a RNA sequence encoding the one or more additional heterologous protein(s) and a RNA sequence encoding a viral structural protein or a portion thereof; and (b) a fusion protein comprising a viral matrix (MA) protein and the one or more additional heterologous protein(s).

In some embodiments, the viral structural protein is gag. In some embodiments, the RNA sequence encoding the viral structural protein or a portion thereof in (a) encodes an N-terminal portion of gag. In some embodiments, the RNA sequence encoding the viral structural protein or a portion thereof in (a) comprises the sequence set forth in SEQ ID NO:52. In some embodiments, the viral MA protein in (b) is derived from human immunodeficiency virus (HIV). In some embodiments, the viral MA protein in (b) comprises the sequence set forth in SEQ ID NO:78.

In some embodiments, the RNA sequence encoding a heterologous protein comprises at or at least 2, 5, 6, 10, 12, 15, 20, or 24 binding sites for binding to the RNA-binding protein. In some embodiments, the RNA sequence encoding a heterologous protein comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20, binding sites for binding to the RNA-binding protein. In some embodiments, the RNA sequence encoding a heterologous protein comprises a plurality of binding sites for binding to the RNA-binding protein. In some embodiments, the plurality of binding sites comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 binding sites. In some embodiments, the plurality of binding sites comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20, binding sites.

In some embodiments, each of the binding sites in the plurality of binding sites is separated by a spacer sequence.

In some embodiments, the RNA-binding protein is MS2 coat protein (MS2_{cp}). In some embodiments, the MS2_{cp} is a homodimer. In some embodiments, the MS2_{cp} is a tandem dimer. In some embodiments, the MS2_{cp} comprises the amino acid sequence set forth in SEQ ID NO:79, or an amino acid sequence having at least 85%, 86%, 87%, 88%, 89%, 90%, 91%, 92%, 93%, 94%, 95%, 96%, 97%, 98%, or 99% sequence identity to the amino acid sequence set forth in SEQ ID NO: 79. In some embodiments, the MS2_{cp} comprises the amino acid sequence set forth in SEQ ID NO:79. In some embodiments, the tandem MS2_{cp} comprises the amino acid sequence set forth in SEQ ID NO: 198, or an amino acid sequence having at least 85%, 86%, 87%, 88%, 89%, 90%, 91%, 92%, 93%, 94%, 95%, 96%, 97%, 98%, or 99% sequence identity to the amino acid sequence set forth in SEQ ID NO: 198. In some embodiments, the tandem MS2_{cp} comprises the amino acid sequence set forth in SEQ ID NO: 198. In some embodiments, the RNA-binding protein is MS2_{cp} and the binding site is an MS2_{cp}-binding loop for binding to the MS2_{cp}. In some embodiments, the MS2_{cp}-binding loop comprises any nucleic acid sequence as disclosed in Johansson et al., Seminars in Virology, 1997, 8:176-185, the contents of which are hereby incorporated by reference in its entirety. In some embodiments, the MS2_{cp}-binding loop comprises the RNA sequence transcribed from the nucleic acid set forth in SEQ ID NO: 185. In some embodiments, the MS2_{cp}-binding loop comprises the RNA sequence transcribed from the nucleic acid set forth in SEQ ID NO: 174. In some embodiments, the MS2_{cp}-binding loop comprises the RNA set forth in SEQ ID NO: 208. In some embodiments, the RNA sequence encoding a heterologous protein comprises a plurality of MS2_{cp}-binding loops. In some embodiments, the plurality of MS2_{cp}-binding loops comprises a plurality of MS2_{cp}-binding loops designed as disclosed in, e.g., Wu et al., Genes DeV., 2015, 29:876-886; or Bertrand et al., Molecular Cell, 1998, Vol. 2:437-445, the contents of which is hereby incorporated by reference in their entirety.

In some embodiments, each of the MS2_{cp}-binding loops in the plurality of MS2_{cp}-binding loops is separated by a spacer sequence. In some embodiments, the plurality of MS2_{cp}-binding loops comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops. In some embodiments, the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops. In some embodiments, the plurality of MS2_{cp}-binding loops comprises 6 MS2_{cp}-binding loops. In some embodiments, the plurality of MS2_{cp}-binding loops comprises 12 MS2_{cp}-binding loops. In some embodiments, the plurality of MS2_{cp}-binding loops comprises 12 MS2_{cp}-binding loops. In some embodiments, the plurality of MS2_{cp}-binding loops comprises between 5 and 50 MS2_{cp}-binding loops. In some embodiments, the plurality of MS2_{cp}-binding loops comprises between 5 and 40 MS2_{cp}-binding loops. In some embodiments, the plurality of MS2_{cp}-binding loops comprises between 5 and 30 MS2_{cp}-binding loops. In some embodiments, the plurality of MS2_{cp}-binding loops comprises between 5 and 24 MS2_{cp}-binding loops. In some embodiments, the plurality of MS2_{cp}-binding loops comprises between 10 and 50 MS2_{cp}-binding loops. In some embodiments, the plurality of MS2_{cp}-binding loops comprises between 10 and 40 MS2_{cp}-binding loops. In some embodiments, the plurality of MS2_{cp}-binding loops comprises between 10 and 30 MS2_{cp}-binding loops. In some embodiments, the plurality of MS2_{cp}-binding loops comprises between 10 and 24 MS2_{cp}-binding loops. In some embodiments, each of the plurality of MS2_{cp}-binding loops comprises the RNA sequence transcribed from the nucleic acid set forth in SEQ ID NO: 174. In some embodiments, each of the plurality of MS2_{cp}-binding loops comprises the RNA sequence transcribed from the nucleic acid set forth in SEQ ID NO: 185. In some embodiments, the plurality of MS2_{cp}-binding loops which can further encode heterologous proteins comprise the RNA sequence transcribed from the nucleic acid set forth in one of SEQ ID NOs: 175-178.

In some embodiments, the RNA-binding protein is lambda N protein (λN) or a functional variant thereof. In some embodiments, the λN or a functional variant thereof comprises the amino acid sequence of SEQ ID NO: 187 or an amino acid sequence having at least 90% or 95% sequence identity to the amino acid sequence of SEQ ID NO: 187. In some embodiments, the λN or a functional variant thereof comprises the amino acid sequence of SEQ ID NO: 187. In some embodiments, the λN or a functional variant thereof is a functional variant of λN that exhibits enhanced binding affinity to RNA, such as any RNA binding protein disclosed in Austin et al., J. Am. Chem. Soc., 2002, 124:10966-10967, the contents of which are hereby incorporated by reference in its entirety. In some embodiments, the λN or a functional variant thereof comprises the amino acid sequence of SEQ ID NO: 188 or an amino acid sequence having at least 90% or 95% sequence identity to the amino acid sequence of SEQ ID NO: 188. In some embodiments, the λN or a functional variant thereof comprises the amino acid sequence of SEQ ID NO: 188. In some embodiments, the λN or a functional variant thereof is a variant of λN that exhibits enhanced binding affinity to RNA and comprises the the amino acid sequence of SEQ ID NO: 188 or an amino acid sequence having at least 90% or 95% sequence identity to the amino acid sequence of SEQ ID NO: 188.

In some embodiments, the RNA-binding protein is λN or a functional variant thereof and the binding site is a boxB binding site for binding to the λN or a functional variant thereof. In some embodiments, the boxB binding site comprises the RNA sequence transcribed from the nucleic acid set forth in SEQ ID NO: 186. In some embodiments, the RNA sequence encoding a heterologous protein comprises a plurality of boxB binding sites. In some embodiments, the plurality of boxB binding sites comprises a plurality of boxB binding sites designed as disclosed in, e.g., Pillai et al., RNA, 2004, 10:1518-1525, the contents of which are hereby incorporated by reference in its entirety.

In some embodiments, each of the boxB binding sites in the plurality of boxB binding sites is separated by a spacer sequence. In some embodiments, the plurality of boxB binding sites comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 boxB binding sites. In some embodiments, the plurality of boxB binding sites comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 boxB binding sites. In some embodiments, the plurality of boxB binding sites comprises 5 boxB binding sites. In some embodiments, the plurality of boxB binding sites comprises 10 boxB binding sites. In some embodiments, the plurality of boxB binding sites comprises 15 boxB binding sites. In some embodiments, the plurality of boxB binding sites comprises 20 boxB binding sites. In some embodiments, the plurality of boxB binding sites comprises between 5 and 50 boxB binding sites. In some embodiments, the plurality of boxB binding sites comprises between 5 and 40 boxB binding sites. In some embodiments, the plurality of boxB binding sites comprises between 5 and 30 boxB binding sites. In some embodiments, the plurality of boxB binding sites comprises between 5 and 20 boxB binding sites. In some embodiments, the plurality of boxB binding sites comprises between 10 and 50 boxB binding sites. In some embodiments, the plurality of boxB binding sites comprises between 10 and 40 boxB binding sites. In some embodiments, the plurality of boxB binding sites comprises between 10 and 30 boxB binding sites. In some embodiments, the plurality of boxB binding sites comprises between 10 and 20 boxB binding sites. In some embodiments, the plurality of boxB binding sites which can further encode heterologous proteins comprise the RNA sequence transcribed from the nucleic acid set forth in one of SEQ ID NOs: 179-184.

In some embodiments, the fusion protein is encoded by a nucleic acid sequence selected from the group consisting of SEQ ID NOs: 62 and 150-156. In some embodiments, the fusion protein is encoded by the nucleic acid sequence set forth in SEQ ID NO: 62. In some embodiments, the fusion protein is encoded by the nucleic acid sequence set forth in SEQ ID NO: 150. In some embodiments, the fusion protein is encoded by the nucleic acid sequence set forth in SEQ ID NO: 151. In some embodiments, the fusion protein is encoded by the nucleic acid sequence set forth in SEQ ID NO: 152. In some embodiments, the fusion protein is encoded by the nucleic acid sequence set forth in SEQ ID NO: 153. In some embodiments, the fusion protein is encoded by the nucleic acid sequence set forth in SEQ ID NO: 154. In some embodiments, the fusion protein is encoded by the nucleic acid sequence set forth in SEQ ID NO: 155. In some embodiments, the fusion protein is encoded by the nucleic acid sequence set forth in SEQ ID NO: 156.

In some embodiments, the fusion protein is encoded by a nucleic acid sequence selected from the group consisting of SEQ ID NOs: 134, 157-162, and 190. In some embodiments, the fusion protein is encoded by the nucleic acid sequence set forth in SEQ ID NO: 134. In some embodiments, the fusion protein is encoded by the nucleic acid sequence set forth in SEQ ID NO: 157. In some embodiments, the fusion protein is encoded by the nucleic acid sequence set forth in SEQ ID NO: 158. In some embodiments, the fusion protein is encoded by the nucleic acid sequence set forth in SEQ ID NO: 159. In some embodiments, the fusion protein is encoded by the nucleic acid sequence set forth in SEQ ID NO: 160. In some embodiments, the fusion protein is encoded by the nucleic acid sequence set forth in SEQ ID NO: 161. In some embodiments, the fusion protein is encoded by the nucleic acid sequence set forth in SEQ ID NO: 162. In some embodiments, the fusion protein is encoded by the nucleic acid sequence set forth in SEQ ID NO: 190.

In some embodiments, the fusion protein comprises an amino acid sequence selected from the group consisting of SEQ ID NOs: 74 and 191-197. In some embodiments, the fusion protein comprises the amino acid sequence set forth in SEQ ID NO: 74. In some embodiments, the fusion protein comprises the amino acid sequence set forth in SEQ ID NO: 191. In some embodiments, the fusion protein comprises the amino acid sequence set forth in SEQ ID NO: 192. In some embodiments, the fusion protein comprises the amino acid sequence set forth in SEQ ID NO: 193. In some embodiments, the fusion protein comprises the amino acid sequence set forth in SEQ ID NO: 194. In some embodiments, the fusion protein comprises the amino acid sequence set forth in SEQ ID NO: 195. In some embodiments, the fusion protein comprises the amino acid sequence set forth in SEQ ID NO: 196. In some embodiments, the fusion protein comprises the amino acid sequence set forth in SEQ ID NO: 197.

In some embodiments, the heterologous protein is a genome-modifying protein. In some embodiments, the genome-modifying protein comprises a recombinant nuclease, a nickase, an integrase, reverse transcriptase, or a combination thereof. In some embodiments, the genome-modifying protein comprises a zinc-finger nuclease (ZFN), a transcription-activator like effector nucleases (TALEN), or a CRISPR-associated (Cas) protein. In some embodiments, the genome-modifying protein is a Cas protein. In some embodiments, the genome-modifying protein is Cas9. In some embodiments, the genome-modifying protein is saCas9. In some embodiments, the genome-modifying protein is spCas9. In some embodiments, the genome-modifying protein is cpf1. In some embodiments, a Cas protein comprises a core Cas protein. Exemplary Cas core proteins include, but are not limited to, Cas1, Cas2, Cas3, Cas4, Cas5, Cas6, Cas7, Cas8, Cas9, Cas12a (also known as Cpf1), Cas12b, Cas1212, Cas13, and Mad7. In some embodiments, a Cas protein comprises a Cas protein of an E. coli subtype (also known as CASS2).

Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1. Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, the heterologous protein is a tumor neoepitope. In some embodiments, the heterologous protein is a viral Spike(s) glycoprotein. In some embodiments, the heterologous protein is a protein from Zika virus, optionally Zika virus prM-E protein; tuberculosis;

respiratory syncytial virus (RSV), optionally RSV fusion (RSV-F) protein; influenza virus, optionally influenza virus hemagglutinin (HA); rabies virus, optionally rabies virus glycoprotein (RABV-G); human cytolomegalovirus (CMV); hepatitis C virus; human immunodeficiency virus 1 (HIV-1), and Streptococcus. In some embodiments, the heterologous protein is an antibody or an antigen-binding fragment thereof.

In some embodiments, the lipid particle comprises a guide RNA (gRNA) in the lumen. In some embodiments, the gRNA is a single guide RNA (sgRNA).

In some embodiments, the lipid particle is pseudotyped with a viral envelope glycoprotein. In some embodiments, the viral envelope glycoprotein is a VSV-G protein or a functional variant thereof. In some embodiments, the viral envelope glycoprotein is a Cocal virus G protein or a functional variant thereof. In some embodiments, the viral envelope glycoprotein is an Alphavirus fusion protein (e.g. Sindbis virus) or a functional variant thereof. In some embodiments, the viral envelope glycoprotein is a Paramyxoviridae fusion protein (e.g., a Morbillivirus or a Henipavirus) or a functional variant thereof. In some embodiments, the viral envelope glycoprotein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof. In some embodiments, the viral envelope glycoprotein is a Henipavirus fusion protein (e.g., Nipah virus, Hendra virus, Cedar virus, Kumasi virus, Mòjiāng virus, Langya virus) or a functional variant thereof.

In some embodiments, the viral envelope glycoprotein comprises one or more modifications to reduce binding to its native receptor. In some embodiments, the viral envelope glycoprotein comprises a Nipah virus F glycoprotein (NiV-F) or a biologically active portion thereof and a Nipah virus G glycoprotein (NiV-G) or a biologically active portion thereof. In some embodiments, the NiV-G or the biologically active portion thereof is a wild-type NiV-G protein or a functionally active variant or biologically active portion thereof.

In some embodiments, the Niv-F or a biologically active portion thereof comprises the amino acid sequence set forth in SEQ ID NO: 144 or 145, or an amino acid sequence having at least 90%, 91%, 92%, 93%, 94%, 95%, 96%, 97%, 98%, or 99% sequence identity to the amino acid sequence set forth in SEQ ID NO: 144 or 145; and/or the Niv-G or a biologically active portion thereof comprises the amino acid sequence set forth in SEQ ID NO: 146 or 147, or an amino acid sequence having at least 90%, 91%, 92%, 93%, 94%, 95%, 96%, 97%, 98%, or 99% sequence identity to the amino acid sequence set forth in SEQ ID NO: 146 or 147. In some embodiments, the Niv-F or a biologically active portion thereof comprises the amino acid sequence set forth in SEQ ID NO: 144 or 145, or an amino acid sequence having at least 90%, 91%, 92%, 93%, 94%, 95%, 96%, 97%, 98%, or 99% sequence identity to the amino acid sequence set forth in SEQ ID NO: 144 or 145; and the Niv-G or a biologically active portion thereof comprises the amino acid sequence set forth in SEQ ID NO: 146 or 147, or an amino acid sequence having at least 90%, 91%, 92%, 93%, 94%, 95%, 96%, 97%, 98%, or 99% sequence identity to the amino acid sequence set forth in SEQ ID NO: 146 or 147. In some embodiments, the Niv-F or a biologically active portion thereof comprises the amino acid sequence set forth in SEQ ID NO: 144 or 145; and/or the Niv-G or a biologically active portion thereof comprises the amino acid sequence set forth in SEQ ID NO: 146 or 147. In some embodiments, the Niv-F or a biologically active portion thereof comprises the amino acid sequence set forth in SEQ ID NO: 144 or 145; and the Niv-G or a biologically active portion thereof comprises the amino acid sequence set forth in SEQ ID NO: 146 or 147.

In some embodiments, the NiV-G protein or the biologically active portion is truncated and lacks up to 40 contiguous amino acid residues at or near the N-terminus of the wild-type NiV-G protein. In some embodiments, the NiV-G protein or the biologically active portion has a 5 amino acid truncation at or near the N-terminus of the wild-type NiV-G protein, optionally wherein the NiV-G protein or the biologically active portion thereof has the amino acid sequence set forth in SEQ ID NO: 12, or a sequence of amino acids that exhibits at least at or about 80%, 85%, 90% or 95% sequence identity to the sequence set forth in SEQ ID NO: 12. In some embodiments, the NiV-G protein or the biologically active portion has a 10 amino acid truncation at or near the N-terminus of the wild-type NiV-G protein, optionally wherein the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44. In some embodiments, the NiV-G protein or the biologically active portion has a 15 amino acid truncation at or near the N-terminus of the wild-type NiV-G protein, optionally wherein the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45. In some embodiments, the NiV-G protein or the biologically active portion has a 20 amino acid truncation at or near the N-terminus of the wild-type NiV-G protein, optionally wherein the NiV-G protein or the biologically active portion thereof has the amino acid sequence set forth in SEQ ID NO: 13, or a sequence of amino acids that exhibits at least at or about 80%, 85%, 90% or 95% sequence identity to the sequence set forth in SEQ ID NO:13. In some embodiments, the NiV-G protein or the biologically active portion has a 25 amino acid truncation at or near the N-terminus of the wild-type NiV-G protein, optionally wherein the NiV-G protein or the biologically active portion thereof has the amino acid sequence set forth in SEQ ID NO: 14, or a sequence of amino acids that exhibits at least at or about 80%, 85%, 90% or 95% sequence identity to the sequence set forth in SEQ ID NO: 14. In some embodiments, the NiV-G protein or the biologically active portion has a 30 amino acid truncation at or near the N-terminus of the wild-type NiV-G protein, optionally wherein the NiV-G protein or the biologically active portion thereof has the amino acid sequence set forth in SEQ ID NO: 43, or a sequence of amino acids that exhibits at least at or about 80%, 85%, 90% or 95% sequence identity to the sequence set forth in SEQ ID NO:43. In some embodiments, the NiV-G protein or the biologically active portion has a 34 amino acid truncation at or near the N-terminus of the wild-type NiV-G protein, optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.

In some embodiments, the NiV-G-protein or the biologically active portion thereof is a mutant NiV-G protein that exhibits reduced binding to Ephrin B2 or Ephrin B3. In some embodiments, the mutant NiV-G protein or the biologically active portion comprises one or more amino acid substitutions corresponding to amino acid substitutions selected from the group consisting of E501A, W504A, Q530A and E533A with reference to numbering set forth in SEQ ID NO: 4.

In some embodiments, the mutant NiV-G protein or the biologically active portion comprises the amino acid sequence set forth in SEQ ID NO: 17 or a sequence of amino acids that exhibits at least at or about 80%, 85%, 90% or 95% sequence identity to the sequence set forth in SEQ ID NO: 17. In some embodiments, the NiV-G protein or the biologically active portion has the amino acid sequence set forth in SEQ ID NO: 18 or a sequence of amino acids that exhibits at least at or about 80%, 85%, 90% or 95% sequence identity to the sequence set forth in SEQ ID NO: 18. In some embodiments, the NiV-F protein or the biologically active portion thereof is a wild-type NiV-F protein or is a functionally active variant or a biologically active portion thereof. In some embodiments, the NiV-F protein or the biologically active portion thereof has a 20 amino acid truncation at or near the C-terminus of the wild-type NiV-F protein, optionally wherein the NiV-F protein or the biologically active portion thereof comprises the sequence set forth in SEQ ID NO: 20 or a sequence of amino acids that exhibits at least at or about 80%, 85%, 90% or 95% sequence identity to the sequence set forth in SEQ ID NO: 20.

In some embodiments, the NiV-F protein or the biologically active portion thereof comprises: i) a 20 amino acid truncation at or near the C-terminus of the wild-type NiV-F protein; and ii) a point mutation on an N-linked glycosylation site. In some embodiments, the NiV-F protein or the biologically active portion thereof comprises the sequence set forth in SEQ ID NO: 15, or a sequence of amino acids that exhibits at least at or about 80%, 85%, 90% or 95% sequence identity to the sequence set forth in SEQ ID NO: 15. In some embodiments, the NiV-F protein or the biologically active portion thereof has a 22 amino acid truncation at or near the C-terminus of the wild-type NiV-F protein. In some embodiments, the NiV-F protein or the biologically active portion thereof comprises the sequence set forth in SEQ ID NO: 16, 19, or 21 or a sequence of amino acids that exhibits at least at or about 80%, 85%, 90% or 95% sequence identity to the sequence set forth in SEQ ID NO: 16, 19, or 21.

In some embodiments, the NiV-F protein or the biologically active portion thereof comprises the amino acid sequence set forth in SEQ ID NO:21, or a sequence of amino acids that exhibits at least at or about 80%, 85%, 90% or 95% sequence identity to the sequence set forth in SEQ ID NO:21. In some embodiments, the Niv-G protein comprises the amino acid sequence set forth in SEQ ID NO: 17, and the Niv-F protein comprises the amino acid sequence set forth in SEQ ID NO:21.

In some embodiments, the lipid particle comprises a targeting moiety. In some embodiments, the targeting moiety binds to a target cell. In some embodiments, the targeting moiety is a single domain antibody (sdAb). In some embodiments, the sdAb can be human or humanized. In some embodiments, the sdAb is a VHH. In some embodiments, the targeting moiety is a single chain molecule. In some embodiments, the targeting moiety is a single chain variable fragment (scFv). In particular embodiments, the targeting moiety contains an antibody variable sequence(s) that is human or humanized.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cadiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the targeting moiety binds to any one of CD3, CD8, CD4, asialoglycoprotein receptor 2 (ASGR2), transmembrane 4 L6 family member 5 (TM4SF5), low density lipoprotein receptor (LDLR) or asialoglycoprotein 1 (ASGR1).

In some embodiments, the targeting moiety is selected from the group consisting of a CD3-binding agent, a CD8-binding agent, and a CD4-binding agent. In some embodiments, the targeting moiety is a CD3-binding agent, optionally an anti-CD3 antibody or an antigen-binding fragment.

In some embodiments, the targeting moiety is a CD8-binding agent, optionally an anti-CD8 antibody or an antigen-binding fragment. In some embodiments, the targeting moiety is a CD4-binding agent, optionally an anti-CD4 antibody or an antigen-binding fragment. In some embodiments, the targeting moiety is exposed on the surface of the lipid particle. In some embodiments, the targeting moiety is fused to a transmembrane domain incorporated into the bilayer of the lipid particle.

In some embodiments, the lipid particle is a retroviral vector or a retroviral-like particle. In some embodiments, the retroviral vector or the retroviral-like particle is replication-deficient. In some embodiments, the lipid particle does not comprise reverse transcriptase or does not comprise reverse transcriptase activity. In some embodiments, the lipid particle does not comprise a protein with reverse transcriptase activity. In some embodiments, the lipid particle does not comprise reverse transcriptase. In some embodiments, the lipid particle comprises non-functional reverse transcriptase. In some embodiments, the reverse transcriptase is mutated. In some embodiments, the retroviral vector or retroviral-like particle comprises a RNA that is a self-inactivating lentiviral vector genome. In some embodiments, the retroviral vector or retroviral-like particle comprises a RNA comprising a 3′LTR, and the 3′ LTR does not comprise a functional U3 domain. In some embodiments, the U3 domain comprises a deletion.

In some embodiments, the lipid particle is a retroviral particle, and the retroviral particle is a lentiviral particle. In some embodiments, the lipid particle is a retrovirus-like particle (VLP).

In some embodiments, the lipid bilayer is derived from a host cell. In some embodiments, the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell. Also provided herein is a method of producing a lipid particle comprising a lipid bilayer enclosing a lumen and a viral transfer plasmid, comprising: (1) providing a host cell comprising (a) a nucleic acid sequence encoding a fusion protein comprising a viral envelope glycoprotein and an RNA binding protein; (b) a nucleic acid sequence encoding a heterologous protein; and (2) culturing the host cell under conditions to induce packaging of the lipid particle. In some embodiments, the host cells further comprises a nucleic acid sequence encoding a protein selected from the group consisting of gag, pol, Rev, Tat, or a combination thereof. In some embodiments, the fusion protein comprises, from a 5′ to 3′ direction: the viral envelope glycoprotein and the RNA binding protein. In some embodiments, the viral envelope glycoprotein is a VSV-G protein or a functional variant thereof. In some embodiments, the RNA binding protein is a MS2 coat protein (MS2_{cp}). In some embodiments, the viral envelope glycoprotein is derived from human immunodeficiency virus (HIV). In some embodiments, the RNA binding protein is a lambda N protein (λN) or a functional variant thereof.

Also provided herein is a method of producing a lipid particle comprising a lipid bilayer enclosing a lumen and a viral transfer plasmid, comprising (1) providing a host cell comprising (a) a nucleic acid sequence encoding a fusion protein comprising a viral matrix (MA) protein and an RNA binding protein; (b) a nucleic acid sequence encoding a heterologous protein; and (c) a nucleic acid sequence encoding a protein selected from the group consisting of gag, pol, Rev, Tat, a viral envelope glycoprotein, or a combination thereof; and (2) culturing the host cell under conditions to induce packaging of the lipid particle. In some embodiments, the RNA binding protein is a MS2 coat protein (MS2_{cp}). In some embodiments, the RNA binding protein is a lambda N protein (λN) or a functional variant thereof.

Also provided herein is a method of producing a lipid particle comprising a lipid bilayer enclosing a lumen and a viral transfer plasmid, comprising: (1) providing a host cell comprising (a) a nucleic acid sequence encoding a fusion protein comprising a viral matrix (MA) protein and a MS2 coat protein (MS2_{cp}); (b) a nucleic acid sequence encoding a heterologous protein; and (c) a nucleic acid sequence encoding a protein selected from the group consisting of gag, pol, Rev, Tat, a viral envelope glycoprotein, or a combination thereof; and (2) culturing the host cell under conditions to induce packaging of the lipid particle.

In some embodiments, the fusion protein comprises, from a 5′ to 3′ direction: the viral MA protein and MS2_{cp}. In some embodiments, the nucleic acid sequence encoding a heterologous protein comprises a MS2_{cp}-binding loop. In some embodiments, the nucleic acid sequence encoding a heterologous protein comprises 12 MS2_{cp}-binding loops. In some embodiments, the nucleic acid sequence encoding a heterologous protein comprises 24 MS2_{cp}-binding loops. In some embodiments, the nucleic acid sequence encoding a heterologous protein comprises a MS2_{cp}-binding loop, optionally at or at least 2, 3, 4, 5, 6, 7, 8, 9, 10, 11, 12, 13, 14, 15, 16, 17, 18, 19, 20, 21, 22, 23, or 24 MS2_{cp}-binding loops. In some embodiments, the MS2_{cp}-binding loop comprises the RNA sequence transcribed from the nucleic acid set forth in SEQ ID NO: 174 or 185. In some embodiments, the MS2_{cp}-binding loop comprises the RNA sequence set forth in SEQ ID NO: 208. In some embodiments, each of the MS2_{cp}-binding loops comprises the RNA sequence transcribed from the nucleic acid set forth in SEQ ID NO: 174 or 185.

In some embodiments, the fusion protein comprises, from a 5′ to 3′ direction: the viral MA protein and λN or a functional variant thereof. In some embodiments, the fusion protein comprises, from a 5′ to 3′ direction: the viral envelope glycoprotein and λN or a functional variant thereof.

In some embodiments, the λN or a functional variant thereof comprises the amino acid sequence of SEQ ID NO: 187 or an amino acid sequence having at least 90% or 95% sequence identity to the amino acid sequence of SEQ ID NO: 187. In some embodiments, the λN or a functional variant thereof comprises the amino acid sequence of SEQ ID NO: 187. In some embodiments, the λN or a functional variant thereof comprises the amino acid sequence of SEQ ID NO: 188 or an amino acid sequence having at least 90% or 95% sequence identity to the amino acid sequence of SEQ ID NO: 188. In some embodiments, the λN or a functional variant thereof comprises the amino acid sequence of SEQ ID NO: 188. In some embodiments, the nucleic acid sequence encoding a heterologous protein comprises a boxB binding site for binding to λN or a functional variant thereof, optionally at or at least 2, 3, 4, 5, 6, 7, 8, 9, 10, 11, 12, 13, 14, 15, 16, 17, 18, 19, 20, 21, 22, 23, or 24 boxB binding sites. In some embodiments, the boxB binding site comprises the RNA sequence transcribed from the nucleic acid set forth in SEQ ID NO: 186. In some embodiments, the nucleic acid sequence encoding a heterologous protein comprises a plurality of boxB binding sites for binding to λN or a functional variant thereof, and the plurality of boxB binding sites comprises the RNA sequence transcribed from a nucleic acid sequence selected from the group consisting of SEQ ID NOs: 179-184.

In some embodiments, the viral MA protein is derived from human immunodeficiency virus (HIV). In some embodiments, the viral MA protein comprises the sequence set forth in SEQ ID NO:78. In some embodiments, MS2_{cp} comprises the sequence set forth in SEQ ID NO:79. In some embodiments, the fusion protein comprises the sequence set forth in SEQ ID NO:74. In some embodiments, the viral envelope glycoprotein is VSV-G.

In some embodiments, the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

In some embodiments, the nucleic acid sequence in (b) comprises a 5′ promoter. In some embodiments, the nucleic acid sequence in (c) comprises a 5′ promoter. In some embodiments, the promoter is a cytomegalovirus (CMV) promoter.

Also provided herein is a lipid particle produced by any of the methods provided herein. Also provided herein is a composition comprising a lipid particle provided herein.

Also provided herein is a method of introducing a heterologous protein into a target cell, the method comprising contacting the target cell with a lipid particle or composition provided herein. Also provided herein is a method of genetically engineering a target cell, the method comprising contacting the target cell with a lipid particle or composition provided herein.

In some embodiments, the contacting is in vitro or ex vivo. In some embodiments, the contacting is in vivo.

Also provided herein is a DNA sequence encoding a viral matrix (MA) protein, a MS2 coat protein (MS2_{cp}), and a cleavage site between the portions of the DNA sequence encoding the MA protein and the MS2_{cp}.

In some embodiments, the DNA sequence encodes a fusion protein comprising, from 5′ to 3′, the viral MA protein and MS2_{cp}. In some embodiments, the encoded MS2_{cp} comprises a MS2_{cp}-binding loop. In some embodiments, the encoded MS2_{cp} comprises 12 MS2_{cp}-binding loops. In some embodiments, the encoded MS2_{cp} comprises 24 MS2_{cp}-binding loops. In some embodiments, the encoded viral MA protein is derived from human immunodeficiency virus (HIV). In some embodiments, the encoded viral MA protein comprises the sequence set forth in SEQ ID NO:78. In some embodiments, the encoded MS2_{cp} comprises the sequence set forth in SEQ ID NO:79. In some embodiments, the encoded fusion protein comprises the sequence set forth in SEQ ID NO:74.

Also provided herein is a DNA sequence encoding a viral matrix (MA) protein, an RNA binding protein, and a cleavage site between the portions of the DNA sequence encoding the MA protein and the RNA binding protein. In some embodiments, the DNA sequence encodes a fusion protein comprising, from 5′ to 3′, the viral MA protein and RNA binding protein. In some embodiments, the RNA binding protein is a MS2 coat protein (MS2_{cp}). In some embodiments, the MS2_{cp} comprises the sequence set forth in SEQ ID NO:79. In some embodiments, the RNA binding protein is a lambda N protein (λN) or a functional variant thereof. In some embodiments, the λN or a functional variant thereof comprises the amino acid sequence of SEQ ID NO: 187 or an amino acid sequence having at least 90% or 95% sequence identity to the amino acid sequence of SEQ ID NO: 187. In some embodiments, the λN or a functional variant thereof comprises the amino acid sequence of SEQ ID NO: 187. In some embodiments, the λN or a functional variant thereof comprises the amino acid sequence of SEQ ID NO: 188 or an amino acid sequence having at least 90% or 95% sequence identity to the amino acid sequence of SEQ ID NO: 188. In some embodiments, the λN or a functional variant thereof comprises the amino acid sequence of SEQ ID NO: 188.

Also provided herein is a DNA sequence encoding a viral envelope glycoprotein, an RNA binding protein, and a cleavage site between the portions of the DNA sequence encoding the viral envelope glycoprotein and the RNA binding protein. In some embodiments, the fusion protein comprises, from a 5′ to 3′ direction: the viral envelope glycoprotein and the RNA binding protein. In some embodiments, the viral envelope glycoprotein is a VSV-G protein or a functional variant thereof. In some embodiments, the RNA binding protein is a MS2 coat protein (MS2_{cp}). In some embodiments, the viral envelope glycoprotein is derived from human immunodeficiency virus (HIV). In some embodiments, the MS2_{cp} comprises the sequence set forth in SEQ ID NO:79. In some embodiments, the RNA binding protein is a lambda N protein (λN) or a functional variant thereof.

In some embodiments, the DNA sequence comprises a nucleic acid sequence selected from the group consisting of SEQ ID NOs: 62 and 150-156. In some embodiments, the DNA sequence comprises the nucleic acid sequence set forth in SEQ ID NO: 62. In some embodiments, the DNA sequence comprises the nucleic acid sequence set forth in SEQ ID NO: 150. In some embodiments, the DNA sequence comprises the nucleic acid sequence set forth in SEQ ID NO: 151. In some embodiments, the DNA sequence comprises the nucleic acid sequence set forth in SEQ ID NO: 152. In some embodiments, the DNA sequence comprises the nucleic acid sequence set forth in SEQ ID NO: 153. In some embodiments, the DNA sequence comprises the nucleic acid sequence set forth in SEQ ID NO: 154. In some embodiments, the DNA sequence comprises the nucleic acid sequence set forth in SEQ ID NO: 155. In some embodiments, the DNA sequence comprises the nucleic acid sequence set forth in SEQ ID NO: 156.

In some embodiments, the DNA sequence comprises a nucleic acid sequence selected from the group consisting of SEQ ID NOs: 134 and 157-162. In some embodiments, the DNA sequence comprises the nucleic acid sequence set forth in SEQ ID NO: 134. In some embodiments, the DNA sequence comprises the nucleic acid sequence set forth in SEQ ID NO: 157. In some embodiments, the DNA sequence comprises the nucleic acid sequence set forth in SEQ ID NO: 158. In some embodiments, the DNA sequence comprises the nucleic acid sequence set forth in SEQ ID NO: 159. In some embodiments, the DNA sequence comprises the nucleic acid sequence set forth in SEQ ID NO: 160. In some embodiments, the DNA sequence comprises the nucleic acid sequence set forth in SEQ ID NO: 161. In some embodiments, the DNA sequence comprises the nucleic acid sequence set forth in SEQ ID NO: 162.

In some embodiments, the DNA sequence comprises a nucleic acid sequence encoding an amino acid sequence selected from the group consisting of SEQ ID NOs: 74 and 191-197. In some embodiments, the DNA sequence comprises a nucleic acid sequence encoding the amino acid sequence set forth in SEQ ID NO: 74. In some embodiments, the DNA sequence comprises a nucleic acid sequence encoding the amino acid sequence set forth in SEQ ID NO: 191. In some embodiments, the DNA sequence comprises a nucleic acid sequence encoding the amino acid sequence set forth in SEQ ID NO: 192. In some embodiments, the DNA sequence comprises a nucleic acid sequence encoding the amino acid sequence set forth in SEQ ID NO: 193. In some embodiments, the DNA sequence comprises a nucleic acid sequence encoding the amino acid sequence set forth in SEQ ID NO: 194. In some embodiments, the DNA sequence comprises a nucleic acid sequence encoding the amino acid sequence set forth in SEQ ID NO: 195. In some embodiments, the DNA sequence comprises a nucleic acid sequence encoding the amino acid sequence set forth in SEQ ID NO: 196. In some embodiments, the DNA sequence comprises a nucleic acid sequence encoding the amino acid sequence set forth in SEQ ID NO: 197.

Also provided herein is a vector comprising a DNA sequence provided herein. Also provided here in a mammalian cell comprising a DNA sequence or a vector provided herein. In some embodiments, the mammalian cell comprises a viral nucleic acid, wherein the viral nucleic acid lacks one or more genes involved in viral replication. In some embodiments, the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3); a nucleic acid encoding a viral envelope protein; and/or a nucleic acid encoding a viral packaging protein selected from one or more of gag, pol, rev and tat. In some embodiments, the mammaial cell comprises a RNA sequence encoding a heterologous protein. In some embodiments, the mammaial cell comprises a guide RNA (gRNA).

Also provided herein is a transfer plasmid comprising a promoter operably linked to a nucleic acid sequence encoding a fusion protein comprising a viral matrix (MA) protein and a MS2 coat protein (MS2_{cp}). In some embodiments, the transfer plasmid is a lentiviral transfer plasmid.

In some embodiments, sequences disclosed herein are expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

### 3. Tethering of Protein

Provided herein is a lipid particle comprising a lipid bilayer enclosing a lumen a fusion protein comprising a viral matrix (MA) protein and a heterologous protein.

Also provided herein is a lipid particle comprising a lipid bilayer enclosing a lumen; a viral matrix (MA) protein; and a heterologous protein, wherein the heterologous protein is incorporated into the lipid particle as a fusion protein with the viral MA protein.

In some embodiments, the fusion protein dissociates within the lipid particle, such that the viral MA protein and the heterologous protein are present in the lipid particle as separate polypeptides. In some embodiments, the fusion protein comprising the viral MA protein and the heterologous protein is cleaved, such that the viral MA protein and the heterologous protein are present in the lipid particle as separate polypeptides. In some embodiments, the fusion particle is cleaved at a cleavage sequence. In some embodiments, the cleavage sequence is located between the viral MA protein and the heterologous protein. In some embodiments, the cleavage sequence comprises the amino acid residues PIVQ (SEQ ID NO: 140). In some embodiments, the cleavage sequence is the amino acid residues PIVQ (SEQ ID NO: 140). In some embodiments, the cleavage sequence comprises the sequence set forth in SEQ ID NO: 128. In some embodiments, the cleavage sequence is the sequence set forth in SEQ ID NO:128. In some embodiments, the cleavage sequence comprises the amino acid residues SQNYPIVQ (SEQ ID NO: 128). In some embodiments, the cleavage sequence is the amino acid residues SQNYPIVQ (SEQ ID NO: 128).

In some embodiments, the viral MA protein reversibly binds to the lipid bilayer. In some embodiments, the fusion protein comprises, from a 5′ to 3′ direction: the viral MA protein and the heterologous protein. In some embodiments, the viral MA protein is derived from human immunodeficiency virus (HIV). In some embodiments, the viral MA protein comprises the sequence set forth in SEQ ID NO:78. In some embodiments, the viral MA protein may be fused to a heterologous protein. In some embodiments, the sequence encoding the viral MA protein which may be fused to a heterologous protein is set forth in SEQ ID NO: 207.

In some embodiments, the lipid particle comprises a transfer plasmid encoding a guide RNA (gRNA). In some embodiments, the gRNA is a single guide RNA (sgRNA). In some embodiments, the gRNA is under the control of a U6 promoter.

In some embodiments, the heterologous protein is a first heterologous protein and the lipid particle further comprises one or more additional heterologous protein(s), wherein the lipid particle comprises: (a) a RNA comprising a RNA sequence encoding the one or more additional heterologous protein(s) and a RNA sequence encoding a viral structural protein or a portion thereof; and/or (b) a viral matrix (MA) protein, a MS2 coat protein (MS2_{cp}), and a RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}. In some embodiments, the heterologous protein is a first heterologous protein and the lipid particle further comprises one or more additional heterologous protein(s), wherein the lipid particle comprises a RNA comprising a RNA sequence encoding the one or more additional heterologous protein(s) and a RNA sequence encoding a viral structural protein or a portion thereof. In some embodiments, the heterologous protein is a first heterologous protein and the lipid particle further comprises one or more additional heterologous protein(s), wherein the lipid particle comprises a viral matrix (MA) protein, a MS2 coat protein (MS2_{cp}), and a RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}. In some embodiments, the heterologous protein is a first heterologous protein and the lipid particle further comprises one or more additional heterologous protein(s), wherein the lipid particle comprises: (a) a RNA comprising a RNA sequence encoding the one or more additional heterologous protein(s) and a RNA sequence encoding a viral structural protein or a portion thereof; and (b) a viral matrix (MA) protein, a MS2 coat protein (MS2_{cp}), and a RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}.

In some embodiments, the viral structural protein in (a) is gag. In some embodiments, the RNA sequence encoding the viral structural protein or a portion thereof in (a) encodes an N-terminal portion of gag. In some embodiments, the RNA sequence encoding the viral structural protein or a portion thereof in (a) comprises the sequence set forth in SEQ ID NO:52. In some embodiments, the viral MA protein in (b) is derived from HIV. In some embodiments, the viral MA protein in (b) comprises the sequence set forth in SEQ ID NO:78. In some embodiments, MS2_{cp} in (b) comprises the sequence set forth in SEQ ID NO:79. In some embodiments, the fusion protein of (b) comprises the sequence set forth in SEQ ID NO:74.

In some embodiments, the heterologous protein is a genome-modifying protein. In some embodiments, the genome-modifying protein comprises a recombinant nuclease, a nickase, an integrase, reverse transcriptase, or a combination thereof. In some embodiments, the genome-modifying protein comprises a zinc-finger nuclease (ZFN), a transcription-activator like effector nucleases (TALEN), or a CRISPR-associated (Cas) protein. In some embodiments, the genome-modifying protein is a Cas protein. In some embodiments, the genome-modifying protein is Cas9. In some embodiments, the genome-modifying protein is saCas9. In some embodiments, the genome-modifying protein is spCas9. In some embodiments, the genome-modifying protein is cpf1. In some embodiments, a Cas protein comprises a core Cas protein. Exemplary Cas core proteins include, but are not limited to, Cas1, Cas2, Cas3, Cas4, Cas5, Cas6, Cas7, Cas8, Cas9, Cas12a (also known as Cpf1), Cas12b, Cas1212, Cas13, and Mad7. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1. Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, the heterologous protein is a tumor neoepitope. In some embodiments, the heterologous protein is a viral Spike(s) glycoprotein. In some embodiments, the heterologous protein is a protein from Zika virus, optionally Zika virus prM-E protein; tuberculosis;

respiratory syncytial virus (RSV), optionally RSV fusion (RSV-F) protein; influenza virus, optionally influenza virus hemagglutinin (HA); rabies virus, optionally rabies virus glycoprotein (RABV-G); human cytolomegalovirus (CMV); hepatitis C virus; human immunodeficiency virus 1 (HIV-1), and Streptococcus. In some embodiments, the heterologous protein is an antibody or an antigen-binding fragment thereof.

In some embodiments, the lipid particle comprises a guide RNA (gRNA) in the lumen. In some embodiments, the gRNA is a single guide RNA (sgRNA).

In some embodiments, the lipid particle is pseudotyped with a viral envelope glycoprotein. In some embodiments, the viral envelope glycoprotein is a VSV-G protein or a functional variant thereof. In some embodiments, the viral envelope glycoprotein is a Cocal virus G protein or a functional variant thereof. In some embodiments, the viral envelope glycoprotein is an Alphavirus fusion protein (e.g. Sindbis virus) or a functional variant thereof. In some embodiments, the viral envelope glycoprotein is a Paramyxoviridae fusion protein (e.g., a Morbillivirus or a Henipavirus) or a functional variant thereof. In some embodiments, the viral envelope glycoprotein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof. In some embodiments, the viral envelope glycoprotein is a Henipavirus fusion protein (e.g., Nipah virus, Hendra virus, Cedar virus, Kumasi virus, Mòjiāng virus, Langya virus) or a functional variant thereof.

In some embodiments, the viral envelope glycoprotein comprises one or more modifications to reduce binding to its native receptor. In some embodiments, the viral envelope glycoprotein comprises a Nipah virus F glycoprotein (NiV-F) or a biologically active portion thereof and a Nipah virus G glycoprotein (NiV-G) or a biologically active portion thereof. In some embodiments, the NiV-G or the biologically active portion thereof is a wild-type NiV-G protein or a functionally active variant or biologically active portion thereof.

In some embodiments, the Niv-F or a biologically active portion thereof comprises the amino acid sequence set forth in SEQ ID NO: 145, or an amino acid sequence having at least 90%, 91%, 92%, 93%, 94%, 95%, 96%, 97%, 98%, or 99% sequence identity to the amino acid sequence set forth in SEQ ID NO: 145; and/or the Niv-G or a biologically active portion thereof comprises the amino acid sequence set forth in SEQ ID NO: 147, or an amino acid sequence having at least 90%, 91%, 92%, 93%, 94%, 95%, 96%, 97%, 98%, or 99% sequence identity to the amino acid sequence set forth in SEQ ID NO: 147. In some embodiments, the Niv-F or a biologically active portion thereof comprises the amino acid sequence set forth in SEQ ID NO: 145, or an amino acid sequence having at least 90%, 91%, 92%, 93%, 94%, 95%, 96%, 97%, 98%, or 99% sequence identity to the amino acid sequence set forth in SEQ ID NO: 145; and the Niv-G or a biologically active portion thereof comprises the amino acid sequence set forth in SEQ ID NO: 147, or an amino acid sequence having at least 90%, 91%, 92%, 93%, 94%, 95%, 96%, 97%, 98%, or 99% sequence identity to the amino acid sequence set forth in SEQ ID NO: 147. In some embodiments, the Niv-F or a biologically active portion thereof comprises the amino acid sequence set forth in SEQ ID NO: 145; and/or the Niv-G or a biologically active portion thereof comprises the amino acid sequence set forth in SEQ ID NO: 147. In some embodiments, the Niv-F or a biologically active portion thereof comprises the amino acid sequence set forth in SEQ ID NO: 145; and the Niv-G or a biologically active portion thereof comprises the amino acid sequence set forth in SEQ ID NO: 147.

In some embodiments, the NiV-G protein or the biologically active portion is truncated and lacks up to 40 contiguous amino acid residues at or near the N-terminus of the wild-type NiV-G protein. In some embodiments, the NiV-G protein or the biologically active portion has a 5 amino acid truncation at or near the N-terminus of the wild-type NiV-G protein, optionally wherein the NiV-G protein or the biologically active portion thereof has the amino acid sequence set forth in SEQ ID NO: 12, or a sequence of amino acids that exhibits at least at or about 80%, 85%, 90% or 95% sequence identity to the sequence set forth in SEQ ID NO: 12. In some embodiments, the NiV-G protein or the biologically active portion has a 10 amino acid truncation at or near the N-terminus of the wild-type NiV-G protein, optionally wherein the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44. In some embodiments, the NiV-G protein or the biologically active portion has a 15 amino acid truncation at or near the N-terminus of the wild-type NiV-G protein, optionally wherein the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45. In some embodiments, the NiV-G protein or the biologically active portion has a 20 amino acid truncation at or near the N-terminus of the wild-type NiV-G protein, optionally wherein the NiV-G protein or the biologically active portion thereof has the amino acid sequence set forth in SEQ ID NO: 13, or a sequence of amino acids that exhibits at least at or about 80%, 85%, 90% or 95% sequence identity to the sequence set forth in SEQ ID NO:13. In some embodiments, the NiV-G protein or the biologically active portion has a 25 amino acid truncation at or near the N-terminus of the wild-type NiV-G protein, optionally wherein the NiV-G protein or the biologically active portion thereof has the amino acid sequence set forth in SEQ ID NO: 14, or a sequence of amino acids that exhibits at least at or about 80%, 85%, 90% or 95% sequence identity to the sequence set forth in SEQ ID NO: 14. In some embodiments, the NiV-G protein or the biologically active portion has a 30 amino acid truncation at or near the N-terminus of the wild-type NiV-G protein, optionally wherein the NiV-G protein or the biologically active portion thereof has the amino acid sequence set forth in SEQ ID NO: 43, or a sequence of amino acids that exhibits at least at or about 80%, 85%, 90% or 95% sequence identity to the sequence set forth in SEQ ID NO:43. In some embodiments, the NiV-G protein or the biologically active portion has a 34 amino acid truncation at or near the N-terminus of the wild-type NiV-G protein, optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.

In some embodiments, the NiV-G-protein or the biologically active portion thereof is a mutant NiV-G protein that exhibits reduced binding to Ephrin B2 or Ephrin B3. In some embodiments, the mutant NiV-G protein or the biologically active portion comprises one or more amino acid substitutions corresponding to amino acid substitutions selected from the group consisting of E501A, W504A, Q530A and E533A with reference to numbering set forth in SEQ ID NO: 4.

In some embodiments, the mutant NiV-G protein or the biologically active portion comprises the amino acid sequence set forth in SEQ ID NO: 17 or a sequence of amino acids that exhibits at least at or about 80%, 85%, 90% or 95% sequence identity to the sequence set forth in SEQ ID NO: 17. In some embodiments, the NiV-G protein or the biologically active portion has the amino acid sequence set forth in SEQ ID NO: 18 or a sequence of amino acids that exhibits at least at or about 80%, 85%, 90% or 95% sequence identity to the sequence set forth in SEQ ID NO: 18. In some embodiments, the NiV-F protein or the biologically active portion thereof is a wild-type NiV-F protein or is a functionally active variant or a biologically active portion thereof. In some embodiments, the NiV-F protein or the biologically active portion thereof has a 20 amino acid truncation at or near the C-terminus of the wild-type NiV-F protein, optionally wherein the NiV-F protein or the biologically active portion thereof comprises the sequence set forth in SEQ ID NO: 20 or a sequence of amino acids that exhibits at least at or about 80%, 85%, 90% or 95% sequence identity to the sequence set forth in SEQ ID NO: 20.

In some embodiments, the NiV-F protein or the biologically active portion thereof comprises: i) a 20 amino acid truncation at or near the C-terminus of the wild-type NiV-F protein; and ii) a point mutation on an N-linked glycosylation site. In some embodiments, the NiV-F protein or the biologically active portion thereof comprises the sequence set forth in SEQ ID NO: 15, or a sequence of amino acids that exhibits at least at or about 80%, 85%, 90% or 95% sequence identity to the sequence set forth in SEQ ID NO: 15. In some embodiments, the NiV-F protein or the biologically active portion thereof has a 22 amino acid truncation at or near the C-terminus of the wild-type NiV-F protein. In some embodiments, the NiV-F protein or the biologically active portion thereof comprises the sequence set forth in SEQ ID NO: 16, 19, or 21 or a sequence of amino acids that exhibits at least at or about 80%, 85%, 90% or 95% sequence identity to the sequence set forth in SEQ ID NO: 16, 19, or 21.

In some embodiments, the NiV-F protein or the biologically active portion thereof comprises the amino acid sequence set forth in SEQ ID NO:21, or a sequence of amino acids that exhibits at least at or about 80%, 85%, 90% or 95% sequence identity to the sequence set forth in SEQ ID NO:21. In some embodiments, the Niv-G protein comprises the amino acid sequence set forth in SEQ ID NO: 17, and the Niv-F protein comprises the amino acid sequence set forth in SEQ ID NO:21.

In some embodiments, the lipid particle comprises a targeting moiety. In some embodiments, the targeting moiety binds to a target cell. In some embodiments, the targeting moiety is a single domain antibody (sdAb). In some embodiments, the sdAb can be human or humanized. In some embodiments, the sdAb is a VHH. In some embodiments, the targeting moiety is a single chain molecule. In some embodiments, the targeting moiety is a single chain variable fragment (scFv). In particular embodiments, the targeting moiety contains an antibody variable sequence(s) that is human or humanized.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cadiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the targeting moiety binds to any one of CD3, CD8, CD4, asialoglycoprotein receptor 2 (ASGR2), transmembrane 4 L6 family member 5 (TM4SF5), low density lipoprotein receptor (LDLR) or asialoglycoprotein 1 (ASGR1).

In some embodiments, the targeting moiety is selected from the group consisting of a CD3-binding agent, a CD8-binding agent, and a CD4-binding agent. In some embodiments, the targeting moiety is a CD3-binding agent, optionally an anti-CD3 antibody or an antigen-binding fragment.

In some embodiments, the targeting moiety is a CD8-binding agent, optionally an anti-CD8 antibody or an antigen-binding fragment. In some embodiments, the targeting moiety is a CD4-binding agent, optionally an anti-CD4 antibody or an antigen-binding fragment. In some embodiments, the targeting moiety is exposed on the surface of the lipid particle. In some embodiments, the targeting moiety is fused to a transmembrane domain incorporated into the bilayer of the lipid particle.

In some embodiments, the lipid particle is a retroviral vector or a retroviral-like particle. In some embodiments, the retroviral vector or the retroviral-like particle is replication-deficient. In some embodiments, the lipid particle does not comprise reverse transcriptase or does not comprise reverse transcriptase activity. In some embodiments, the lipid particle does not comprise a protein with reverse transcriptase activity. In some embodiments, the lipid particle does not comprise reverse transcriptase. In some embodiments, the lipid particle comprises non-functional reverse transcriptase. In some embodiments, the reverse transcriptase is mutated. In some embodiments, the retroviral vector or retroviral-like particle comprises a RNA that is a self-inactivating lentiviral vector genome. In some embodiments, the retroviral vector or retroviral-like particle comprises a RNA comprising a 3′LTR, and the 3′ LTR does not comprise a functional U3 domain. In some embodiments, the U3 domain comprises a deletion.

In some embodiments, the lipid particle is a retroviral particle, and the retroviral particle is a lentiviral particle. In some embodiments, the lipid particle is a retrovirus-like particle (VLP).

In some embodiments, the lipid bilayer is derived from a host cell. In some embodiments, the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

Also provided herein is a method of producing a lipid particle comprising a lipid bilayer enclosing a lumen and a viral transfer plasmid, comprising: (1) providing a host cell comprising (a) a nucleic acid sequence encoding a fusion protein comprising viral matrix (MA) protein and a heterologous protein; and (b) a nucleic acid sequence encoding a protein selected from the group consisting of gag, pol. Rev, Tat, a viral envelope glycoprotein, or a combination thereof; and (2) culturing the host cell under conditions to induce packaging of the lipid particle.

In some embodiments, the fusion protein comprises, from a 5′ to 3′ direction: the viral MA protein and the heterologous protein. In some embodiments, the viral MA protein is derived from human immunodeficiency virus (HIV). In some embodiments, the viral MA protein comprises the sequence set forth in SEQ ID NO:78. In some embodiments, the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

In some embodiments, the nucleic acid sequence in (b) comprises a 5′ promoter. In some embodiments, the promoter is a cytomegalovirus (CMV) promoter.

Also provided herein is a lipid particle produced by any of the methods provided herein. Also provided herein is a composition comprising a lipid particle provided herein.

Also provided herein is a method of introducing a heterologous protein into a target cell, the method comprising contacting the target cell with a lipid particle or composition provided herein. Also provided herein is a method of genetically engineering a target cell, the method comprising contacting the target cell with a lipid particle or composition provided herein.

In some embodiments, the contacting is in vitro or ex vivo. In some embodiments, the contacting is in vivo.

Also provided herein is a DNA sequence encoding a viral matrix (MA) protein and a heterologous protein. In some embodiments, the DNA sequence encodes a fusion protein comprising, from 5′ to 3′, the viral MA protein and the heterologous protein. In some embodiments, the encoded viral MA protein is derived from human immunodeficiency virus (HIV). In some embodiments, the encoded viral MA protein comprises the sequence set forth in SEQ ID NO:78.

Also provided herein is a vector comprising a DNA sequence provided herein. Also provided here in a mammalian cell comprising a DNA sequence or a vector provided herein. In some embodiments, the mammalian cell comprises a viral nucleic acid, wherein the viral nucleic acid lacks one or more genes involved in viral replication. In some embodiments, the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3); a nucleic acid encoding a viral envelope protein; and/or a nucleic acid encoding a viral packaging protein selected from one or more of gag, pol, rev and tat. In some embodiments, the mammaial cell comprises a RNA sequence encoding a heterologous protein. In some embodiments, the mammaial cell comprises a guide RNA (gRNA).

Also provided herein is a transfer plasmid comprising a promoter operably linked to a nucleic acid sequence encoding a viral matrix (MA) protein and a heterologous protein. In some embodiments, the transfer plasmid is a lentiviral transfer plasmid.

In some embodiments, sequences disclosed herein are expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

### B. Types of Lipid Particles

Provided herein are lipid particles. In some embodiments, the lipid particles are viral-based particles or cell-based particles.

### 1. Viral-Based Particles

Provided herein are viral-based particles derived from a virus, including those derived from retroviruses, such as lentiviruses. In some embodiments, the lipid particle's bilayer of amphipathic lipids is or comprises the viral envelope. In some embodiments, the lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the lipid particle's lumen or cavity comprises a viral nucleic acid, e.g., a retroviral nucleic acid, e.g., a lentiviral nucleic acid. In some embodiments, the viral nucleic acid may be a viral genome. In some embodiments, the lipid particle further comprises one or more viral non-structural proteins, e.g., in its cavity or lumen. In some embodiments, the viral-based particle is or comprises a virus-like particle (VLP). In some embodiments, the VLP does not comprise any viral genetic material. In some embodiments, the viral-based particle does not contain any virally derived nucleic acids or viral proteins, such as viral structural proteins.

Biological methods for introducing a heterologous agent to a host cell include the use of DNA and RNA vectors. DNA and RNA vectors can also be used to house and deliver polynucleotides and polypeptides. Viral vectors and virus like particles, and especially retroviral vectors, have become the most widely used method for inserting genes into mammalian, e.g., human cells. Other viral vectors and virus like particles can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

Retroviruses and retroviral genomes are known in the art. For example, reference genomes of retroviruses are known and provided by the NCBI, including for Abelson murine leukemia virus (NC_001499), avian carcinoma virus (NC_001402), avian leukemia virus (NC_015116), avian leukosis virus-RSA (NC_001408), avian myeloblatosis virus (NC_043404), avian myelocytomatosis virus (NC_001866), avian sarcoma virus CT10 (NC_038922), baboon endogenous virus strain M7 (NC_022517), bovine immunodeficiency virus (NC_001413), bovine leukemia virus (NC_001414), bovine retrovirus CH15 (NC_029852), caprine arthritis encephalitis virus (NC_) 001463, chick syncytial virus (NC_), desmodus rotundus endongenous retrovirus (NC_027117), endogenous langur type D retrovirus PO-1-Lu (NC_043193), enzootic nasal tumour virus of goats (NC_004994), equine infectious anemia virus (NC_001450), feline immunodeficiency virus (NC_001482), feline leukemia virus (NC_001940), feline sarcoma virus (NC_038923), Finkel-Biskis-Jinkins murine sarcoma virus (NC_038858), Friend murine leukemia virus (NC_001362), Moloney murine leukemia virus (NC_001501), Murine type C retrovirus (NC_001702), Fujinami sarcoma virus (NC_001403), Gibbon ape leukemia virus (NC_001885), Harvey murine sarcoma virus (NC_038668), Human T-cell leukemia virus type I (NC_001436), primate T-lymphotropic virus 1 (NC_000858), Human T-lymphotropic virus 2 (NC_001488), Simian T-lymphotropic virus 2 (NC_001815), Human T-lymphotropic virus 4 (NC_011800), human immunodeficiency virus 1 (NC_001802), human immunodeficiency virus 2 (NC_001722), Jaagsiekte sheep retrovirus (NC_001494), Jembrana disease virus (NC_001654), Kirsten murine sarcoma virus (NC_043426), Koala retrovirus (NC_039228), Mason-Pfizer monkey virus (NC_001550), Moloney murine sarcoma virus (NC_001502), mouse mammary tumor virus (NC_001503), murine osteosarcoma virus (NC_001506), Mus musculus mobilized endogenous polytropic provirus (NC_029853), ovine enzootic nasal tumor virus (NC_007015), ovine lentivirus (NC_001511), porcine endogenous retrovirus E (NC_003059), preXMRV-1 (NC_) 007815, primate T-lymphotropic virus 3 (NC_003323), Puma lentivirus 14 (NC_038669), RD114 lentiviral (NC_009889), reticuloendotheliosis virus (NC_006934), rous sarcoma virus (NC_001407), simian T-cell lymphotropic virus 6 (NC_011546), simian immunodeficiency virus (NC_001549), simian immunodeficiency virus SIV-mnd 32 (NC_004455), simian retrovirus 4 (NC_014474), simian retrovirus 8 (NC_031326), snakehead retrovirus (NC_001724), Synder-Theilen feline sarcoma virus (NC_043382), Spleen focus-forming virus (NC_001500), squirrel monkey retrovirus (NC_001514), UR2 sarcoma virus (NC_001618), Visna-maedi virus (NC_001452), Walleye dermal sarcoma virus (NC_001867), Walleye epidermal hyperplasia virus 1 (NC_043194), Walleye epidermal hyperplasia virus 2 (NC_043195), Woolly monkey sarcoma virus (NC_009424), Y37 sarcoma virus (NC_008094), African green monkey simian foamy virus (NC_010820), bovine foamy virus (NC_001831), brown greater galago prosimian foamy virus (NC_039023), Central chimpanzee simian foamy virus (NC_039024), Eastern chimpanzee simian foamy virus (NC_039025), equine foamy virus (NC_002201), feline foamy virus (NC_039242), Guenon simian foamy virus (NC_043445), Japanese macaque simian foamy virus (NC_039026), Puma feline foamy virus (NC_039022), simian foamy virus (NC_001364), simian foamy virus pongo pygmacus pygmacus (NC_039085), spider monkey simian foamy virus (NC_039027), squirrel monkey simian foamy virus (NC_039028), Western lowland gorilla simian foamy virus (NC_039029), white-tufted-car marmoset simian foamy virus (NC_039030), yellow-breasted capuchin simian foamy virus (NC_039031), Atlantic salmon swim bladder sarcoma virus (NC_007654), avian endogenous retrovirus EAV-HP (NC_005947), citrus endogenous pararetrovirus (NC_0231563), human endogenous retrovirus K113 (NC_022518), saesbycol virus (NC_040462, NC_040463, and NC_040461), and Xenopus laevis endogenous retrovirus Xen 1 (NC_010955). It is understood that these reference genomes may vary from individual isolates of the indicated species.

In some embodiments, the retrovirus is a lentivirus. Examples of lentiviruses include Bovine immunodeficiency virus, Caprine arthritis encephalitis virus, Equine infectious anemia virus, Feline immunodeficiency virus, Human immunodeficiency virus 1, Human immunodeficiency virus 2, Jembrana disease virus, Puma lentivirus, Simian immunodeficiency virus, and Visna-maedi virus.

In some embodiments, the retrovirus is a gamma retrovirus. Examples of gamma retroviruses include Chick syncytial virus, Feline leukemia virus, Finkel-Biskis-Jinkins murine sarcoma virus, Gardner-Arnstein feline sarcoma virus, Gibbon ape leukemia virus, Guinea pig type-C oncovirus, Hardy-Zuckerman feline sarcoma virus, Harvey murine sarcoma virus, Kirsten murine sarcoma virus, Koala retrovirus, Moloney murine sarcoma virus, Murine leukemia virus (e.g., Moloney murine leukemia virus, Abelson murine leukemia virus, Rauscher murine leukemia virus, and Friend murine leukemia virus), Porcine type-C oncovirus, Reticuloendotheliosis virus, Snyder-Theilen feline sarcoma virus, Trager duck spleen necrosis virus, Viper retrovirus, and Woolly monkey sarcoma virus.

In some embodiments, the viral particles or virus-like particles bilayer of amphipathic lipids is or comprises lipids derived from an infected host cell. In some embodiments, the lipid bilayer is a viral envelope. In some embodiments, the viral particles or virus-like particles envelope is obtained from a host cell. In some embodiments, the viral particles or virus-like particles envelope is obtained by the viral capsid from the source cell plasma membrane. In some embodiments, the lipid bilayer is obtained from a membrane other than the plasma membrane of a host cell. In some embodiments, the viral particles or virus-like particles envelope lipid bilayer is embedded with viral proteins, including viral glycoproteins.

In some embodiments, one or more infective units of viral particles or virus-like particles, e.g. retroviral particles or retroviral-like particles, are administered to the subject. In some embodiments, at least 1, 10, 100, 1000, 10⁴, 10⁵, 10⁶, 10⁷, 10⁸, 10⁹, 10¹⁰, 10¹¹, 10¹², 10¹³, or 10¹⁴, infective units per kg are administered to the subject. In some embodiments at least 1, 10, 100, 1000, 10⁴, 10⁵, 10⁶, 10⁷, 10⁸, 10⁹, 10¹⁰, 10¹¹, 10¹², 10¹³, or 10¹⁴, infective units per target cell per ml of blood are administered to the subject.

a. Viral Vector Particles

In some embodiments, the lipid particle is or comprises a virus or a viral vector, e.g., a retrovirus or retroviral vector, e.g., a lentivirus or lentiviral vector. In some embodiments, the virus or viral vector is recombinant. For instance, the viral particle may be referred to as a recombinant virus or a recombinant viral vector, which are used interchangeably. In some embodiments, the lipid particle is a recombinant lentivirus vector particle.

In some embodiments, a lipid particle comprises a lipid bilayer comprising a retroviral vector comprising an envelope. For instance, in some embodiments, the bilayer of amphipathic lipids is or comprises the viral envelope. The viral envelope may comprise a viral envelope protein (i.e., a fusogen) that is endogenous to the virus or is a pseudotyped fusogen. In some embodiments, the viral vector's lumen or cavity comprises a viral nucleic acid, e.g., a retroviral nucleic acid, e.g., a lentiviral nucleic acid. The viral nucleic acid may be a viral genome. In some embodiments, the viral vector may further comprises one or more viral non-structural proteins, e.g., in its cavity or lumen. In some embodiments, the virus based vector particles are lentivirus. In some embodiments, the lentiviral vector particle is Human Immunodeficiency Virus-1 (HIV-1).

In some aspects, the viral vector particle is limited in the number of polynucleotides that can be packaged. In some embodiments, nucleotides encoding polypeptides to be packaged can be modified such that they retain functional activity with fewer nucleotides in the coding region than that which encodes for the wild-type peptide. Such modifications can include truncations, or other deletions. In some embodiments, more than one polypeptide can be expressed from the same promoter, such that they are fusion polypeptides. In some embodiments, the insert size to be packaged (i.e., viral genome, or portions thereof; or heterologous polynucleotides as described) can be between 500-1000, 1000-2000, 2000-3000, 3000-4000, 4000-5000, 5000-6000, 6000-7000, or 7000-8000 nucleotides in length. In some embodiments, the insert can be over 8000 nucleotides, such as 9000, 10,000, or 11,000 nucleotides in length.

In some embodiments, the viral vector particle, such as retroviral vector particle, comprises one or more of gag polyprotein, polymerase (e.g., pol), integrase (e.g., a functional or non-functional variant), protease, and a fusogen. In some embodiments, the lipid particle further comprises rev. In some embodiments, one or more of the aforesaid proteins are encoded in the retroviral genome (i.e., the insert as described above), and in some embodiments, one or more of the aforesaid proteins are provided in trans, e.g., by a helper cell, helper virus, or helper plasmid. In some embodiments, the lipid particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments, the lipid particle nucleic acid further comprises a retroviral cis-acting RNA packaging element, and a cPPT/CTS element. In some embodiments the lipid particle nucleic acid further comprises one or more insulator element. In some embodiments, the recognition sites are situated between the poly A tail sequence and the WPRE.

In some embodiments, the lipid particle comprises supramolecular complexes formed by viral proteins that self-assemble into capsids. In some embodiments, the lipid particle is a viral particle derived from viral capsids. In some embodiments, the lipid particle is a viral particle derived from viral nucleocapsids. In some embodiments, the lipid particle comprises nucleocapsid-derived that retain the property of packaging nucleic acids.

In some embodiments, the lipid particle packages nucleic acids from host cells carrying one or more viral nucleic acids (e.g. retroviral nucleic acids) during the expression process. In some embodiments, the nucleic acids do not encode any genes involved in virus replication. In particular embodiments, the lipid particle is a virus-based particle, e.g. retrovirus particle such as a lentivirus particle, that is replication defective.

In some cases, the lipid particle is a viral particle that is morphologically indistinguishable from the wild type infectious virus. In some embodiments, the viral particle presents the entire viral proteome as an antigen. In some embodiments, the viral particle presents only a portion of the proteome as an antigen.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other heterologous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretro virus. In some embodiments the retrovirus is a Deltaretro virus.

In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

A viral vector can comprise a nucleic acid molecule (e.g., a transfer plasmid) that includes virus-derived nucleic acid elements that typically facilitate transfer of a nucleic acid molecule (e.g. heterologous nucleic acid per se or nucleic acid encoding an heterologous agent) or integration into the genome of a cell or to a viral particle that mediates nucleic acid transfer. Viral vector particles will typically include various viral components and sometimes also host cell components in addition to nucleic acid(s). A viral vector can comprise a virus or viral particle capable of transferring a nucleic acid into a cell (e.g. heterologous nucleic acid per se or nucleic acid encoding an heterologous agent), or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked nucleic acid) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex. With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane. In some embodiments, the viral vector does not contain reverse transcriptase (RT), such that it is reverse-transcriptase deficient.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell, such as nucleic acid encoding an heterologous agent as described herein. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′) R-U5-one or more first nucleotide sequences-U3-R (3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or combination, codon optimization may be used, e.g., the gene encoding the heterologous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

It is within the level of a skilled artisan to empirically determine appropriate codon optimization of viral sequences. The strategy for codon optimized sequences, including gag-pol sequences, can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV. HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, a lipid particle provided herein comprises a lipid bilayer enclosing a lumen and a nucleic acid. In some embodiments, the lumen comprises a capsid (CA) that encloses the nucleic acid. In some embodiments, the nucleic acid enclosed by the capsid is RNA, such as the retroviral RNA genome.

The MA protein is a structural protein that associates with the viral envelope to connect the capsid to a viral glycoprotein in the lipid bilayer. In this way, the MA protein links the capsid/core of a viral particle with its envelope. In some embodiments, the lipid particle comprises a matrix (MA) protein in association with the lipid bilayer of the particle. In some embodiments, the lipid particle contains a viral matrix (MA) protein, a MS2 coat protein (MS2_{cp}), and an RNA sequence encoding a heterologous protein(s), wherein the MS2_{cp} is incorporated into the lipid particle as a fusion protein with the viral MA protein. In some embodiments, the RNA sequence encoding the heterologous protein comprises a MS2_{cp}-binding loop for binding to MS2_{cp}. For example, in some embodiments, the RNA sequence encoding the heterologous protein comprises 12 or 24 MS2_{cp}-binding loops for binding to MS2_{cp}, which tether the RNA sequence encoding the heterologous protein to inner portion of the lipid bilayer. In other embodiments, the lipid particle contains a fusion protein of a MA protein and a heterologous protein, such that the fusion protein is associated with the inner portion of the lipid bilayer.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798): 1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et ah, (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Y] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. In some aspects, provided herein is a replication incompetent (also referred to herein as replication defective) vector particle, that cannot participate in replication in the absence of the packaging cell (i.e., viral vector particles are not produced from the transduced cell). In some aspects, this is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly (A) sequence The 3′ LTR, the 5′ LTR, or both 3′ and 5′ LTRs, may be modified LTRs. Other modifications to the viral vector, i.e., retroviral or lentiviral vector, to render said vector replication incompetent are known in the art.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et ah, 2000, Cell, 101:173, which are herein incorporated by reference in their entireties. During HIV-I reverse transcription, central initiation of the plus-strand DNA at the central polypurine tract (cPPT) and central termination at the central termination sequence (CTS) can lead to the formation of a three-stranded DNA structure: the HIV-I central DNA flap. In some embodiments, the retroviral or lentiviral vector backbones comprise one or more FLAP elements upstream or downstream of the gene encoding the heterologous agent. For example, in some embodiments a transfer plasmid includes a FLAP element, e.g., a FLAP element derived or isolated from HIV-L.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65:1053; and Cullen et al., 1991. Cell 58:423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences (e.g. nucleic acid encoding a heterologous agent) in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the heterologous agent. Transcription termination signals may be found downstream of the polyadenylation signal. In some embodiments, vectors comprise a polyadenylation sequence 3′ of a polynucleotide encoding the heterologous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATT AAA, AGTAAA, a bovine growth hormone polyA sequence (BGHpA), a rabbit b-globin polyA sequence (rPgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding a heterologous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the heterologous agent, a gene encoding the heterologous agent, a WPRE sequence (e.g., for efficient transgene expression), a PPT sequence (e.g., for reverse transcription), an R sequence (e.g., for polyadenylation and termination), and a U5 signal (e.g., for integration).

b. Virus-Like Particles (VLPs)

Provided herein are lipid particles that are derived from virus, such as viral particles or viral-like particles (VLPs), including those that are derived from retroviruses or lentiviruses.

Generally, a VLP is formed by one or more virus-derived structural protein(s) and/or one more virus-derived envelope protein(s). Methods of generating VLPs are described in WO2017068077, which is incorporated herein in its entirety. In some embodiments, the viral envelope may comprise a fusogen, e.g., a fusogen that is endogenous to the virus or a pseudotyped fusogen. The VLPS include those derived from retroviruses or lentiviruses. While VLPs mimic native virion structure, they lack the viral genomic information necessary for independent replication within a host cell. Therefore, in some aspects, VLPs are non-infectious. In particular embodiments, a VLP does not contain a viral genome. In some embodiments, the VLP's bilayer of amphipathic lipids is or comprises the viral envelope. In some embodiments, the lipid particle's bilayer of amphipathic lipids is or comprises lipids derived from a cell. In some embodiments, the targeted lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the targeted lipid particle's lumen or cavity comprises a viral nucleic acid, e.g., a retroviral nucleic acid, e.g., a lentiviral nucleic acid. In some embodiments, the viral nucleic acid may be a viral genome. In some embodiments, the targeted lipid particle further comprises one or more viral non-structural proteins, e.g., in its cavity or lumen.

In some embodiments, the targeted lipid particles is or comprises a virus-like particle (VLP). In some embodiments, the VLP does not comprise an envelope. In some embodiments, the VLP comprises an envelope. In some embodiments, a VLP contains at least one type of structural protein from a virus. In most cases this protein will form a proteinaceous capsid. In some cases the capsid will also be enveloped in a lipid bilayer originating from the cell from which the assembled VLP has been released (e.g. VLPs comprising a human immunodeficiency virus structural protein such as gag). In some embodiments, the VLP further comprises a targeting moiety as an envelope protein within the lipid bilayer.

In some embodiments, the viral particle or virus-like particle, such as retrovirus or retrovirus-like particle, comprises one or more of gag polyprotein, polymerase (e.g., pol), integrase (e.g., a functional or non-functional variant), protease, and a fusogen. In some embodiments, the targeted lipid particle further comprises rev. In some embodiments, one or more of the aforesaid proteins are encoded in the retroviral genome, and in some embodiments, one or more of the aforesaid proteins are provided in trans, e.g., by a helper cell, helper virus, or helper plasmid. In some embodiments, the targeted lipid particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments the targeted lipid particle nucleic acid further comprises one or more insulator element. In some embodiments, the recognition sites are situated between the poly A tail sequence and the WPRE.

In some embodiments, the vector vehicle particle comprises supramolecular complexes formed by viral proteins that self-assemble into capsids. In some embodiments, the vector vehicle particle is a virus-like particle derived from viral capsid proteins. In some embodiments, the vector vehicle particle is a virus-like particle derived from viral nucleocapsid proteins. In some embodiments, the vector vehicle particle comprises nucleocapsid-derived proteins that retain the property of packaging nucleic acids. In some embodiments, the viral-based particles, such as virus-like particles comprises only viral structural glycoproteins among proteins from the viral genome. In some embodiments, the vector vehicle particle does not contain a viral genome.

In some embodiments, the vector vehicle particle packages nucleic acids from host cells during the expression process, such as a nucleic acid encoding a heterologous agent. In some embodiments, the nucleic acids do not encode any genes involved in virus replication. In particular embodiments, the vector vehicle particle is a virus-like particle, e.g. retrovirus-like particle such as a lentivirus-like particle, that is replication defective.

In some embodiments, the vector vehicle particle is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles could be used to deliver therapeutic RNA, in which case functional integrase and/or reverse transcriptase is not required. In some embodiments, the vector particles do not contain reverse transcriptase. In some embodiments, the vector particles could also be used to deliver a therapeutic gene of interest, in which case pol is typically included.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

c. Methods of Generating Viral-Based Particles

Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes. In some embodiments, a lipid particle provided herein contains nucleic acid encoding one or more of gag, pol, env, tat, rev, vif, vpr, vpx, and vpu. In some embodiments, a lipid particle herein contains a genomic viral RNA.

In some embodiments, viral vector particles may be produced in multiple cell culture systems including bacteria, mammalian cell lines, insect cell lines, yeast and plant cells. Exemplary methods for producing viral vector particles are described.

In some embodiments, elements for the production of a viral vector, i.e., a recombinant viral vector such as a replication incompetent lentiviral vector, are included in a packaging cell line or are present on a packaging vector. In some embodiments, viral vectors can include packaging elements, rev, gag, and pol, delivered to the packaging cells line via one or more packaging vectors.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self cleaving viral peptides. In some embodiments, the packaging vector is a packaging plasmid.

Producer cell lines (also called packaging cell lines) include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In embodiments, the packaging cells are 293 cells, 293T cells, or A549 cells.

In some embodiments, a producer cell (i.e., a source cell line) includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles (i.e. a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al. 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the heterologous agent (e.g., a retroviral nucleic acid encoding the heterologous agent) is also integrated into the source cell genome. In some embodiments a nucleic acid encoding the heterologous agent is maintained episomally. In some embodiments a nucleic acid encoding the heterologous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express a heterologous agent. The retrovirus or VLP, may comprise a disabled reverse transcriptase protein, or may not comprise a reverse transcriptase protein. The retrovirus or VLP may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

Typically, modern retroviral vector systems include viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

A virus-like particle (VLP) which comprises a sequence that is devoid of or lacking viral RNA as described in Section II.B.1.b may be the result of removing or eliminating the viral RNA from the sequence. Similar to the viral vector particles disclosed in Section II.B.1.a. VLPs contain a viral outer envelope made from the host cell (i.e., producer cell or source cell) lipid-bi layer as well as at least one viral structural protein. In some embodiments, a viral structural protein refers to any viral protein or fragment thereof which contributes to the structure of the viral core or capsid. Methods of generating VLPs are described in WO2017068077, incorporated by reference herein in its entirety.

Generally, for viral vector particles as described in Section II.B.1.a, expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The VLP could be used to deliver therapeutic RNA, in which case functional integrase and/or reverse transcriptase is not required. These VLPs could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle. In some embodiments, the heterologous RNA binding domain comprises an RNA binding domain derived from a bacteriophage coat protein, a Rev protein, a protein of the U 1 small nuclear ribonucleoprotein particle, a Nova protein, a TF111 A protein, a TIS 11 protein, a trp RNA-binding attenuation protein (TRAP) or a pseudouridine synthase.

In some embodiments, the assembly of a viral based vector vehicle particle (i.e., a VLP) is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the source cell for VLP production comprises one or more plasmids coding for viral structural proteins (e.g., gag, pol) which can package viral particles (i.e., a packaging plasmid). In some embodiments, the sequences coding for at least two of the gag and pol precursors are on the same plasmid. In some embodiments, the sequences coding for the gag and pol precursors are on different plasmids. In some embodiments, the sequences coding for the gag and pol precursors have the same expression signal, e.g., promoter. In some embodiments, the sequences coding for the gag and pol precursors have a different expression signal, e.g., different promoters. In some embodiments, expression of the gag and pol precursors is inducible.

In some embodiments, formation of VLPs or any viral-based particle as described above can be detected by any suitable technique known in the art. Examples of such techniques include, e.g., electron microscopy, dynamic light scattering, selective chromatographic separation and/or density gradient centrifugation.

### 2. Cell-Based Particles

In some embodiments, the lipid particle is a cell-based particle that includes a naturally derived membrane. In some embodiments, the naturally derived membrane includes membrane vesicles prepared from cells or tissues. In some embodiments, the cell-based particle includes a fusogen. In some embodiments, the cell-based particle comprises a vesicle that is obtainable from a cell. In some embodiments, the cell-based particle is a nanovesicle (e.g. a gesicle). In some embodiments, the cell-based particle is a gesicle. In some embodiments, the cell-based particle does not include a viral structural protein or does not include a viral capsid.

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeEa, HEK293, HFF-1, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell.

In some embodiments, the-cell based particle has a density of <1, 1-1.1, 1.05-1.15, 1.1-1.2, 1.15-1.25, 1.2-1.3, 1.25-1.35, or >1.35 g/ml. In some embodiments, the vector vehicle particle composition comprises less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells having a functional nucleus.

In embodiments, the cell-based particle has a size, or the population of vector vehicle particles have an average size, that is less than about 0.01%, 0.05%, 0.1%, 0.5%, 1%, 2%, 3%, 4%, 5%, 10%, 20%, 30%, 40%, 50%, 60%, 70%, 80%, 90%, of that of the source cell.

In some embodiments the cell-based particle is an extracellular vesicle, e.g., a cell based vesicle comprising a membrane that encloses an internal space and has a smaller diameter than the cell from which it is derived. In embodiments the extracellular vesicle has a diameter from 20 nm to 1000 nm. In embodiments the extracellular vesicle has a diameter from 30 nm to 500 nm. In embodiments the extracellular vesicle has a diameter from 40 nm to 250 nm. In embodiments the extracellular vesicle has a diameter from 50 nm to 150 nm. In some embodiments the cell-based particle is a fragment of a cell, a vesicle derived from a cell by direct or indirect manipulation, a vesiculated organelle, or a vesicle produced from a living cell (e.g., by direct plasma membrane budding or fusion of the late endosome with the plasma membrane). In some embodiments the cell-based particle is a nanovesicle (e.g., a gesicle) produced from a living cell (e.g., by direct plasma membrane budding). In embodiments the extracellular vesicle is derived from cultured cells.

In embodiments, the cell based particle is a nanovesicle (e.g., a cell-derived small (e.g., between 20-250 nm in diameter, or between 30-150 nm in diameter) vesicle comprising a membrane that encloses an internal space, and which is generated from said cell by direct or indirect manipulation. In some embodiments, the nanovesicle (e.g., a gesicle) is generated from the cell by overexpression of VSV-G in the cell. In some embodiments, a gesicle is generated from the cell by overexpression of VSV-G in the cell. In some embodiments, the gesicle has a diameter of between 20-250 nm, or between 30-150 nm. Gesicles and the production thereof are described in Mangeot et al., Mol. Ther. (2011) 19 (9): 1656-66, which is incorporated by reference herein in its entirety. The production of nanovesicles can, in some instances, result in the destruction of the source cell. The nanovesicle may comprise a lipid or fatty acid and polypeptide.

In embodiments, the cell-based particle is an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide. Exemplary exosomes and other membrane-enclosed bodies are also described in WO/2017/161010, WO/2016/077639, US20160168572, US20150290343, and US20070298118, each of which is incorporated by reference herein in its entirety.

In some embodiments, the cell based particle is a microvesicle. In some embodiments the microvesicle has a diameter of about 100 nm to about 2000 nm.

In some embodiments, cell-based particles are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, the source cell used to make the cell-based particle will not be available for testing after the vector vehicle particle is made.

In some embodiments, a characteristic of a cell-based particle is described by comparison to a reference cell. In embodiments, the reference cell is the source cell. In embodiments, the reference cell is a HeLa, HEK293, HFF-1, MRC-5, WI-38, IMR 90, IMR 91, PER.C6, HT-1080, or BJ cell. In embodiments, the reference cell is a HEK293 cell. In some embodiments, a characteristic of a population of vector vehicle particles is described by comparison to a population of reference cells, e.g., a population of source cells, or a population of HeLa, HEK293, HFF-1, MRC-5, WI-38, IMR 90, IMR 91, PER.C6, HT-1080, or BJ cells.

### Heterologous Agents

In some embodiments, the lipid particle or a composition comprising the same described herein contains a heterologous agent (e.g., a heterologous protein or nucleic acid encoding the same). In some embodiments, the lipid particle or a composition comprising the same described herein contains a heterologous agent, such as a nucleic acid that encodes a heterologous protein. Thus, in some embodiments, the heterologous agent is a nucleic acid sequence encoding a heterologous protein. In some embodiments, the lipid particle or a composition comprising the same described herein contains a heterologous nucleic acid sequence per se, such as one that does not encode a heterologous protein (e.g., a guide RNA). Thus, ins some embodiments, the heterologous agent is a heterologous nucleic acid. Reference to the nucleic acid or the coding sequence of the nucleic acid encoding the heterologous protein also is referred to herein as a genetic payload. In some embodiments, the lipid particle contains an heterologous agent, such as a heterologous protein. Thus, in some embodiments, the heterologous agent is a heterologous protein. In some embodiments, a heterologous protein, a heterologous nucleic acid, a nucleic acid encoding a heterologous protein, or any combination thereof are present in the lumen of the lipid particle.

In some embodiments, the heterologous agent comprises a nucleic acid encoding a heterologous protein. In some embodiments, the heterologous agent is a nucleic acid encoding a heterologous protein. In some embodiments, the heterologous protein comprises a genome-modifying protein. In some embodiments, the heterologous protein isa genome-modifying protein.

In some embodiments, the heterologous agent is a protein or a nucleic acid (e.g., a DNA, a chromosome (e.g. a human artificial chromosome), an RNA, e.g., an mRNA or miRNA). In some embodiments, the heterologous agent is a nucleic acid (e.g., a DNA, a chromosome (e.g. a human artificial chromosome), an RNA, e.g., an mRNA or miRNA). In some embodiments, the heterologous agent is RNA encoding for a heterologous protein. In some embodiments, the heterologous agent is RNA that does not encode for a heterologous protein (e.g., gRNA). In some embodiments, the heterologous agent comprises or encodes a membrane protein. In some embodiments, the heterologous agent comprises or encodes a therapeutic agent. In some embodiments, the therapeutic agent is chosen from one or more of a protein, e.g., an enzyme, a transmembrane protein, a receptor, or an antibody; a nucleic acid, e.g., DNA, a chromosome (e.g. a human artificial chromosome), RNA, mRNA, siRNA, or miRNA; or a small molecule. In some embodiments, the heterologous agent is a nucleic acid sequence encoding a heterologous protein. In some embodiments, the heterologous protein is or comprises a genome-modifying protein.

In some embodiments, the lipid particle or a composition thereof delivers to a target cell at least 10%, 20%, 30%, 40%, 50%, 60%, 70%, 80%, 90%, 95%, 96%, 97%, 98%, or 99% of the heterologous agent (e.g., a heterologous agent comprising or encoding a genome-modifying protein) comprised by the lipid particle. In some embodiments, the lipid particle, e.g., fusosome, that contacts, e.g., fuses, with the target cell(s) delivers to the target cell an average of at least 10%, 20%, 30%, 40%, 50%, 60%, 70%, 80%, 90%, 95%, 96%, 97%, 98%, or 99% of the heterologous agent (e.g., a heterologous agent comprising or encoding a genome-modifying protein) comprised by the lipid particles, e.g., fusosomes, that contact, e.g., fuse, with the target cell(s). In some embodiments, the lipid particle composition delivers to a target tissue at least 10%, 20%, 30%, 40%, 50%, 60%, 70%, 80%, 90%, 95%, 96%, 97%, 98%, or 99% of the heterologous agent (e.g., a heterologous agent comprising or encoding a genome-modifying protein) comprised by the lipid particle compositions.

In some embodiments, the heterologous agent is not expressed naturally in the cell from which the lipid particle is derived. In some embodiments, the heterologous agent is expressed naturally in the cell from which the lipid particle is derived. In some embodiments, the heterologous agent is loaded into the lipid particle via expression in the cell from which the lipid particle is derived (e.g. expression from DNA or mRNA introduced via transfection, transduction, or electroporation). In some embodiments, the heterologous agent is expressed from DNA integrated into the genome or maintained episomally. In some embodiments, expression of the heterologous agent is constitutive. In some embodiments, expression of the heterologous agent is induced. In some embodiments, expression of the heterologous agent is induced immediately prior to generating the lipid particle. In some embodiments, expression of the heterologous agent is induced at the same time as expression of the fusogen.

In some embodiments, the heterologous agent is loaded into the lipid particle via electroporation into the lipid particle itself or into the cell from which the lipid particle is derived. In some embodiments, the heterologous agent is loaded into the lipid particle via transfection (e.g., of a DNA or mRNA encoding the heterologous agent) into the lipid particle itself or into the cell from which the lipid particle is derived.

In some embodiments, the heterologous agent may include one or more nucleic acid sequences, one or more polypeptides, a combination of nucleic acid sequences and/or polypeptides, one or more organelles, and any combination thereof. In some embodiments, the heterologous agent may include one or more cellular components. In some embodiments, the heterologous agent includes one or more cytosolic and/or nuclear components.

In some embodiments, the lipid particle contains a heterologous agent that is a nucleic acid, or contains a nucleic acid encoding the heterologous agent. In some embodiments, the nucleic acid is operatively linked to a “positive target cell-specific regulatory element” (or positive TCSRE). Exemplary regulatory elements include any of those described in WO2019222403, WO2020014209, WO2020102485, WO2020102503, and WO2020102499, each of which is incorporated herein by reference in its entirety. In some embodiments, the positive TCSRE is a functional nucleic acid sequence. In some embodiments, the positive TCSRE comprises a promoter or enhancer. In some embodiments, the TCSRE is a nucleic acid sequence that increases the level of a heterologous agent in a target cell. In some embodiments, the positive target cell-specific regulatory element comprises a T cell-specific promoter, a T cell-specific enhancer, a T cell-specific splice site, a T cell-specific site extending half-life of an RNA or protein, a T cell-specific mRNA nuclear export promoting site, a T cell-specific translational enhancing site, or a T cell-specific post-translational modification site. In some embodiments, the T cell-specific promoter is a promoter described in Immgen consortium, herein incorporated by reference in its entirety, e.g., the T cell-specific promoter is an IL2RA (CD25), LRRC32, FOXP3, or IKZF2 promoter. In some embodiments, the T cell-specific promoter or enhancer is a promoter or enhancer described in Schmidl et a, Blood. 2014 Apr. 24; 123 (17): e68-78., herein incorporated by reference in its entirety. In some embodiments, the T cell-specific promoter is a transcriptionally active fragment of any of the foregoing. In some embodiments, the T-cell specific promoter is a variant having at least 70%, 75%, 80%, 85%, 90%, 95%, 96%, 97%, 98%, or 99% identity to any of the foregoing.

In some embodiments, the lipid particle contains a heterologous agent that is a nucleic acid or contains a nucleic acid encoding the heterologous agent. In some embodiments, the nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the lipid particle in a non-target cell. In some embodiments, the heterologous agent is operatively linked to a “non-target cell-specific regulatory element” (or NTCSRE). In some embodiments, the NTCSRE comprises a nucleic acid sequence that decreases the level of a heterologous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR3 1, miR363, or miR29c. In some embodiments, the NTCSRE is situated or encoded within a transcribed region encoding the heterologous agent, optionally wherein an RNA produced by the transcribed region comprises the miRNA recognition sequence within a UTR or coding region.

### A. Nucleic Acids

In some embodiments, the heterologous agent may include a nucleic acid. For example, the heterologous agent may comprise RNA to enhance expression of an endogenous protein, or a siRNA or miRNA that inhibits protein expression of an endogenous protein. For example, the endogenous protein may modulate structure or function in the target cells. In some embodiments, the heterologous may include a nucleic acid encoding an engineered protein that modulates structure or function in the target cells. In some embodiments, the heterologous agent is a nucleic acid that targets a transcriptional activator that modulate structure or function in the target cells.

In some embodiments, the nucleic acid is RNA. In some embodiments, the nucleic acid encodes a heterologous protein. In some embodiments, the nucleic acid is a guide RNA (gRNA), such as a single guide RNA (sgRNA). In some embodiments, the nucleic acid is DNA. In some embodiments, the nucleic acid encodes a heterologous protein. In some embodiments, the nucleic acid is a recombinase template. In some embodiments, the nucleic acid is an integrase template.

In some embodiments, a lipid particle described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide. The nucleic acid may include variants, e.g., having an overall sequence identity with a reference nucleic acid of at least 85%, 86%, 87%, 88%, 89%, 90%, 91%, 92%, 93%, 94%, 95%, 96%, 97%, or 99%. In some embodiments, a variant nucleic acid does not share at least one characteristic sequence element with a reference nucleic acid. In some embodiments, a variant nucleic acid shares one or more of the biological activities of the reference nucleic acid. In some embodiments, a nucleic acid variant has a nucleic acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant nucleic acid comprises about 10, about 9, about 8, about 7, about 6, about 5, about 4, about 3, about 2, or about 1 substituted residue as compared to a reference. In some embodiments, a variant nucleic acid comprises a very small number (e.g., fewer than about 5, about 4, about 3, about 2, or about 1) number of substituted, inserted, or deleted, functional residues that participate in a particular biological activity relative to the reference. In some embodiments, a variant nucleic acid comprises not more than about 15, about 12, about 9, about 3, or about 1 addition or deletion, and, in some embodiments, comprises no additions or deletions, as compared to the reference. In some embodiments, a variant nucleic acid comprises fewer than about 27, about 24, about 21, about 18, about 15, about 12, about 9, about 6, about 3, or fewer than about 9, about 6, about 3, or about 2 additions or deletions as compared to the reference.

In some embodiments, the nucleic acid encodes a heterologous protein that includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, integrases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, transposases, DNA polymerases, RNA polymerases, reverse transcriptases, and any combination thereof.

In some embodiments, the heterologous agent includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprograming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences.

In embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155. In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target. In some embodiments, the heterologous agent comprises a shRNA. A shRNA (short hairpin RNA) can comprise a double-stranded structure that is formed by a single self complementary RNA strand, shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H I RNA promoter and the human tRNA-val promoter, or a strong constitutive pol II promoter.

In some embodiments, the nucleic acid encodes a a genome-modifying protein. Exemplary genome-modifying proteins are described in Section III.C.

### B. Polypeptides

In some embodiments, a lipid particle described herein comprises a heterologous agent which is or comprises a protein. In some embodiments, the heterologous agent comprises a heterologous protein. In some embodiments, the heterologous agent is a heterologous protein. In some embodiments, the heterologous protein comprises a genome-modifying protein. In some embodiments, the heterologous protein is a genome-modifying protein. Exemplary genome-modifying proteins are described in Section III.C.

In some embodiments, the protein may include moieties other than amino acids (e.g., may be glycoproteins, proteoglycans, etc.) and/or may be otherwise processed or modified. In some embodiments, the protein can sometimes include more than one polypeptide chain, for example linked by one or more disulfide bonds or associated by other means.

In some embodiments, the protein may contain L-amino acids, D-amino acids, or both and may contain any of a variety of amino acid modifications or analogs. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof. In some embodiments, a polypeptide may include its variants, e.g., having an overall sequence identity with a reference polypeptide of at least 85%, 86%, 87%, 88%, 89%, 90%, 91%, 92%, 93%, 94%, 95%, 96%, 97%, or 99%. In some embodiments, a variant polypeptide does not share at least one characteristic sequence element with a reference polypeptide. In some embodiments, a variant polypeptide shares one or more of the biological activities of the reference polypeptide. In some embodiments, a polypeptide variant has an amino acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide comprises about 10, about 9, about 8, about 7, about 6, about 5, about 4, about 3, about 2, or about 1 substituted residue as compared to a reference. In some embodiments, a variant polypeptide comprises a very small number (e.g., fewer than about 5, about 4, about 3, about 2, or about 1) number of substituted, inserted, or deleted, functional that participate in a particular biological activity relative to the reference. In some embodiments, a variant polypeptide comprises not more than about 5, about 4, about 3, about 2, or about 1 addition or deletion, and, in some embodiments, comprises no additions or deletions, as compared to the reference. In some embodiments, a variant polypeptide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference.

In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, integrases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, transposases, DNA polymerases, RNA polymerases, reverse transcriptases, and any combination thereof.

In some embodiments, the protein targets a protein in the cell for degradation. In some embodiments, the protein targets a protein in the cell for degradation by localizing the protein to the proteasome. In some embodiments, the protein is a wild-type protein. In some embodiments, the protein is a mutant protein.

Exemplary protein heterologous agents are described in the following subsections. In some embodiments, a lipid particle provided herein can include any of such heterologous agents. In particular embodiments, a lipid particle contains a nucleic acid encoding any of such heterologous agents.

In some embodiments, the heterologous agent comprises a cytosolic protein, e.g., a protein that is produced in the recipient cell and localizes to the recipient cell cytoplasm. In some embodiments, the heterologous agent comprises a secreted protein, e.g., a protein that is produced and secreted by the recipient cell. In some embodiments, the heterologous agent comprises a nuclear protein, e.g., a protein that is produced in the recipient cell and is imported to the nucleus of the recipient cell. In some embodiments, the heterologous agent comprises an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

In some embodiments, the heterologous protein is a tumor neoepitope. In some embodiments, the heterologous protein is a viral Spike(s) glycoprotein. In some embodiments, the heterologous protein is a protein from Zika virus, optionally Zika virus prM-E protein; tuberculosis;

respiratory syncytial virus (RSV), optionally RSV fusion (RSV-F) protein; influenza virus, optionally influenza virus hemagglutinin (HA); rabies virus, optionally rabies virus glycoprotein (RABV-G); human cytolomegalovirus (CMV); hepatitis C virus; human immunodeficiency virus 1 (HIV-1), and Streptococcus. In some embodiments, the heterologous protein is an antibody or an antigen-binding fragment thereof.

In some embodiments, the heterologous protein is a protein meant to label or identify the target cell. In some embodiments the heterologous protein is EGFP. In some embodiments, the sequence of the heterologous protein is set forth in SEQ ID NO: 201. In some embodiments, lentivirus vectors are used to deliver mRNA encoding the heterologous protein and viral genomic mRNA. In some embodiments, lentivirus vectors comprising mRNA encoding the heterologous protein and viral genomic mRNA are made by delivering DNA encoding the heterologous protein and viral genomic mRNA to cells. In some embodiments, the DNA encoding the heterologous protein and viral genomic mRNA comprises a sequence that exhibits at least at or about 80%, 85%, 90% or 95% sequence identity to the sequence set forth in SEQ ID NO: 142. In some embodiments, the major splice donor in the viral genomic mRNA is mutated. In some embodiments, the DNA encoding the heterologous protein and viral genomic mRNA with a mutated major splice donor comprises a sequence that exhibits at least at or about 80%, 85%, 90% or 95% sequence identity to the sequence set forth in SEQ ID NO: 143.

In some embodiments, the heterologous protein is encoded in RNA further comprising MS2 stem loops. In some embodiments, a DNA construct is used which encodes a lentiviral vector comprising MS2_{cp}. In some embodiments, RNA comprising a sequence encoding the heterologous protein and MS2 are capable of binding MS2_{cp}. In some embodiments, the DNA construct that drives expression of mRNA with MS2 stem loops and the heterologous protein comprises a sequence that exhibits at least at or about 80%, 85%, 90% or 95% sequence identity to the sequence set forth in SEQ ID NOs: 148.

In some embodiments, the heterologous protein is fused to a protein which binds the interior of the viral particle. In some embodiments, the heterologous protein is fused to a domain which binds the interior of the viral particle. In some embodiments, the heterologous protein is fused to a reversible membrane attachment domain to bind the heterologous protein reversibly to the interior of the viral particle. In some embodiments, the heterologous protein is fused to a membrane attachment domain and is encoded in DNA comprising a sequence that exhibits at least at or about 80%, 85%, 90% or 95% sequence identity to the sequence set forth in SEQ ID NOs: 149.

### C. Genome-Modifying Proteins and Nucleic Acids Encoding the Same

In some embodiments, the heterologous protein is associated with a genome editing technology. Any of a variety of agents associated with gene editing technologies can be included as the heterologous protein, such as for delivery of gene editing machinery to a cell. In some embodiments, the gene editing technology can include systems involving nuclease, nickase, homing, integrase, transposase, recombinase, and/or reverse transcriptase activity. In some embodiments, the gene editing technologies can be used for knock-out or knock-down of genes. In some embodiments, the gene-editing technologies can be used for knock-in or integration of DNA into a region of the genome. In some embodiments, the heterologous protein mediates single-strand breaks (SSB). In some embodiments, the heterologous protein mediates double-strand breaks (DSB), including in connection with non-homologous end-joining (NHEJ) or homology-directed repair (HDR). In some embodiments, the heterologous protein does not mediate SSB. In some embodiments, the heterologous protein does not mediate DSB. In some embodiments, the heterologous protein can be used for DNA base editing or prime-editing. In some embodiments, the heterologous protein can be used for Programmable Addition via Site-specific Targeting Elements (PASTE).

In some embodiments, the heterologous protein is a nuclease for use in gene editing methods. In some embodiments, the heterologous protein is a zinc-finger nucleases (ZFNs), transcription-activator like effector nucleases (TALENs), or a CRISPR-associated (Cas) protein. In some embodiments, the Cas protein is selected from the group consisting of Cas3, Cas9, Cas10, Cas12, and Cas13. In some embodiments, the Cas is a Cas12a (also known as cpf1) from a Prevotella, Francisella novicida, Acidaminococcus sp., Lachnospiraceae bacterium, or Francisella bacteria. In some embodiments, the Cas is a Cas12b from a Bacillus, optionally Bacillus hisashii. In some embodiments, the Cas is Cas9 from Streptococcus pyogenes (SpCas). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9). The Cas9 nuclease can, in some embodiments, be a Cas9 or functional fragment thereof from any bacterial species. See, e.g., Makarova et al. Nature Reviews, Microbiology, 9:467-477 (2011), including supplemental information, hereby incorporated by reference in its entirety.

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110:19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110:19860-19865; Gong et al., 2005, Nat. Struct Mol Biol, Vol. 12:304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339:819-823; Jinek et al., 2012, Science, Vol. 337:816-821; Qi et al., 2013 Cell, Vol. 152:1173-1183). Cas9D10A is even more appealing in terms of target specificity when loci are targeted by paired Cas9 complexes designed to generate adjacent DNA nicks (Ran et al., 2013, Cell, Vol. 154:1380-1389). In some embodiments, the Cas is a nuclease-deficient Cas9 (Qi et al., 2013 Cell, Vol. 152:1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein. In some embodiments, the Cas protein comprises one or more mutations such that the Cas protein is converted into a nickase that is able to cleave only one strand of a double stranded DNA molecule (e.g., a SSB). In some embodiments, the Cas protein is selected from the group consisting of Cas3, Cas4, Cas5, Cas8a, Cas8b, Cas8c, Cas9, Cas10, Cas12. Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas protein is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A. H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of D10A, H840A, H854A, and H863A.

In some embodiments, the Cas protein is selected from the group consisting of Cas3, Cas9, Cas10, Cas12, and Cas13. In some embodiments, the Cas protein is Cas9. In some embodiments, the one or more agent(s) (e.g., the heterologous protein) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene such as at a location within the sense strand or the antisense strand of the target gene that is or includes the cleavage site. In some embodiments, the recombinant nuclease is a Cas protein from any bacterial species, or is a functional fragment thereof. In some embodiments, the Cas protein is Cas9 nuclease. Cas9 can, in some embodiments, be a Cas9 or functional fragment thereof from any bacterial species. See, e.g., Makarova et al. Nature Reviews, Microbiology, 9:467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A. H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A. R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A. M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M763I, and K890N. In some embodiments, the Cas9 is from Streptococcus pyogenes (SpCas9) and comprises one or more mutations selected from the group consisting of E480K, E543D, E1219V, A262T. S409I, M694I, E108G, S217A.

In some embodiments, the Cas9 is from Streptococcus pyogenes (SaCas9). In some embodiments, the SaCas9 is wild type SaCas9. In some embodiments, the SaCas9 comprises one or more mutations in REC3 domain. In some embodiments, the SaCas9 comprises one or more mutations in REC1 domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e. cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas 12a processes both RNase and DNase activity. In some embodiments, the Cas 12a is a split Cas 12a platform, consisting of N-terminal and C-terminal fragments of Cas12a. In some embodiments, the split Cas 12a platform is from Lachnospiraceae bacterium.

In some embodiments, the lipid particle further comprises a polynucleotide per se, i.e. a polynucleotide that does not encode for a heterologous protein. In some embodiments, the polynucleotide per se is associated with a gene editing system. For example, a lipid particle may comprise a guide RNA (gRNA), such as a single guide RNA (sgRNA).

In some embodiments, the one or more agent(s) (e.g., the heterologous protein) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) (e.g., the heterologous protein) can be used in combination with a donor template, e.g., a single-stranded DNA oligonucleotide (ssODN), for HDR-mediated integration of the donor template into the target gene, such as at the targeting sequence. In some embodiments, the one or more agent(s) (e.g., the heterologous protein) can be used in combination with a donor template, e.g., an ssODN, and a guide RNA, e.g., a sgRNA, for HDR-mediated integration of the donor template into the target gene, such as at the targeting sequence. In some embodiments, the one or more agent(s) (e.g., the heterologous protein) can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In particular embodiments, the genome-modifying agent is a Cas protein, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the genome-modifying agent, e.g., Cas9, is targeted to the cleavage site by interacting with a guide RNA, e.g., sgRNA, that hybridizes to a DNA sequence that immediately precedes a Protospacer Adjacent Motif (PAM) sequence. In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., sgRNA. In some embodiments, the cleavage site is situated at a site within the target gene that is homologous to the sequence of the guide RNA, e.g., sgRNA. In some embodiments, the cleavage site is situated approximately 3 nucleotides upstream of the PAM sequence. In some embodiments, the cleavage site is situated approximately 3 nucleotides upstream of the juncture between the guide RNA and the PAM sequence. In some embodiments, the cleavage site is situated 3 nucleotides upstream of the PAM sequence. In some embodiments, the cleavage site is situated 4 nucleotides upstream of the PAM sequence.

In some embodiments, the one or more agent(s) (e.g., the heterologous protein) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, that includes a cleavage site, such as the targeting sequence.

In some embodiments, the method involves introducing, into a cell, one or more agent(s) (e.g., the heterologous protein) capable of inducing a SSB at a cleavage site within the sense strand and a SSB at a cleavage site within the antisense strand of an endogenous target gene in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the one or more agent(s) (e.g., the heterologous protein) capable of inducing a SSB at a cleavage site within the sense strand and a SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing, into a cell, one or more agent(s) (e.g., the heterologous protein) comprising a recombinant nuclease for inducing a SSB at a cleavage site in the sense strand and a SSB at a cleavage site in the antisense strand within an endogenous target gene in the cell. Although, in some embodiments, it is described that “a” “the” recombinant nuclease induces a SSB in the antisense strand a SSB in the sense strand, it is to be understood that this includes situations where two of the same recombinant nuclease is used, such that one of the recombinant nuclease induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the one or more agent(s) capable of inducing a SSB comprise a recombinant nuclease and a first guide RNA, e.g., a first sgRNA, and a second guide RNA, e.g., a second sgRNA.

In some embodiments, the genome-modifying agent is a Cas protein, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a SSB at a cleavage site within the sense strand and a SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site. In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene that includes a cleavage site, such as the targeting sequence.

In some embodiments, the one or more agent(s) capable of inducing a SSB at a cleavage site within the sense strand and a SSB at a cleavage site within the antisense strand involve use of the CRISPR/Cas gene editing system. In some embodiments, the one or more agent(s) comprise a recombinant nuclease.

In some embodiments, the genome-modifying agent is a Cas protein. In some embodiments, the Cas protein comprises one or more mutations such that the Cas protein is converted into a nickase that lacks the ability to cleave both strands of a double stranded DNA molecule. In some embodiments, the Cas protein comprises one or more mutations such that the Cas protein is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the lipid particle further comprises a guide RNA (gRNA), such as a single guide RNA (sgRNA). Thus, in some embodiments, the heterologous agent comprises a guide RNA (gRNA). In some embodiments, the gRNA is a single guide RNA (sgRNA).

In some embodiments, the genome-modifying protein, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the genome-modifying agent, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the genome-modifying agent, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene. In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene.

In some embodiments, the second guide RNA, e.g., second sgNA, that is specific to the sense strand of a target gene of interest used to target the recombinant nuclease, e.g., Cas9, to induce a SSB at a cleavage site within the sense strand of the target gene. In some embodiments, the second guide RNA. e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene; and the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene; and the second guide RNA, e.g., second sgNA, that is specific to the sense strand of a target gene of interest is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target gene that is homologous to a sequence comprised within the first guide RNA, e.g., the first sgRNA. In some embodiments, the cleavage site of the antisense strand is situated at a site within the antisense strand of the target gene that is homologous to a sequence comprised within the first guide RNA, e.g., the first sgRNA. In some embodiments, the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA. In some embodiments, the cleavage site of the sense strand is situated at a site within the sense strand of the target gene that is homologous to a sequence comprised within the first guide RNA, e.g., the first sgRNA; and the cleavage site of the antisense strand is situated at a site within the antisense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the first guide RNA, e.g., the first sgRNA; and the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA; and the cleavage site of the sense strand is situated at a site within the sense strand of the target gene that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the the PAM sequence on the sense strand and the PAM sequence on the antisense strand comprise the same nucleic acid sequence, which can be any PAM sequence disclosed herein. In some embodiments, the the PAM sequence on the sense strand and the PAM sequence on the antisense strand each comprise a different nucleic acid sequence, each of which can be any of the PAM sequences disclosed herein.

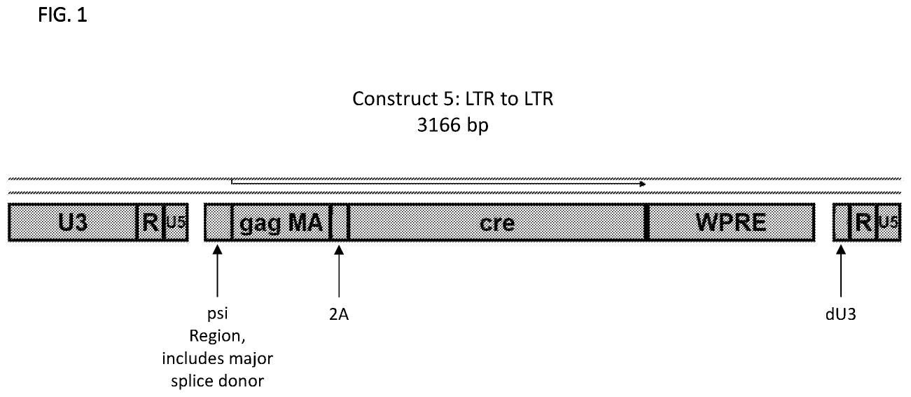

In some embodiments, the PAM sequence that is recognized by a recombinant nuclease, e.g., Cas9, differs depending on the particular recombinant nuclease and the bacterial species it is from Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA. e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene that includes the cleavage site. In some embodiments, the crRNA comprises a nucleotide sequence that is 100% homologous to a portion of the target gene that includes the cleavage site. In some embodiments, the portion of the target gene that includes the cleavage site is a portion of the sense strand of the target gene that includes the cleavage site. In some embodiments, the portion of the target gene that includes the cleavage site is a portion of the antisense strand of the target gene that includes the cleavage site.

In some embodiments, the sgRNA comprises a crRNA sequence that is homologous to a sequence in the target gene that includes the cleavage site. In some embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene that includes the cleavage site. In some embodiments, the crRNA sequence of the first sgRNA has 100% sequence identity to a sequence in the sense strand of the target gene that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/nature13011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of a target gene that includes the cleavage site. In some embodiments, the crRNA is homologous to a portion of the sense strand of the target gene that includes the cleavage site. In some embodiments, the crRNA is homologous to a portion of the antisense strand of the target gene that includes the cleavage site. In some embodiments, the crRNA of the first sgRNA is homologous to a portion of the sense strand of the target gene that includes the cleavage site; and the crRNA of the second sgRNA is homologous to a portion of the antisense strand of the target gene that includes the cleavage site.

In some embodiments, the crRNA is homologous to a portion of the antisense strand of a target gene that includes the cleavage site. In some embodiments, the crRNA is homologous to a portion of the sense strand of the target gene that includes the cleavage site. In some embodiments, the crRNA of the first sgRNA is homologous to a portion of the antisense strand of the target gene that includes the cleavage site; and the crRNA of the second sgRNA is homologous to a portion of the sense strand of the target gene that includes the cleavage site.

In some embodiments, the crRNA is homologous to a portion of a target gene that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene that includes the cleavage site is on the sense strand. In some embodiments, the portion of the target gene that includes the cleavage site is on the antisense strand.

In some embodiments, the crRNA is homologous to a portion, i.e., sequence, in the sense strand or the antisense strand of the target gene that includes the cleavage site and is immediately upstream of the PAM sequence.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, and 20140080216.

In some embodiments, the heterologous protein is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C·G to T·A and adenine base editors (e.g., ABE7.10) that convert target A·T to G·C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U: A base pair, which is then converted to T: A during DNA replication.

In some embodiments, the heterologous protein is or encodes a base editor (e.g., a nucleobase editor). In some embodiments, the heterologous protein is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622. US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the heterologous protein is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the heterologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the heterologous protein is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PERNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the heterologous protein is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in nondividing cells and fewer detectable off-target events.

In some embodiments, the heterologous protein is or encodes one or more polypeptides having an activity selected from the group consisting of: nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

In some embodiments, delivery of the nuclease is by a provided vector encoding the nuclease (e.g. Cas).

In some embodiments, the provided lipid particles contain a nuclease protein and the nuclease protein is directly delivered to a target cell. Methods of delivering a nuclease protein include those as described, for example, in Cai et al. Elife, 2014, 3: e01911 and International patent publication No. WO2017068077. For instance, provided lipid particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid particle (e.g. lentiviral vector particle, VLP, or gesicle). For instance, a chimeric Cas9-protein fusion with the structural GAG protein can be packaged inside a lipid particle. In some embodiments, the fusion protein is a cleavable fusion protein between (i) a viral structural protein (e.g. GAG) and (ii) a nuclease protein (e.g. Cas protein, such as Cas9). In some embodiments, the fusion protein is a cleavable fusion protein between (i) a viral matrix (MA) protein and (ii) a nuclease protein (e.g. Cas protein, such as Cas9). In some embodiments, the particle contains a nuclease protein (e.g., Cas protein, such as Cas 9) immediately downstream of the gag start codon.

In some embodiments, the provided lipid particles contain mRNA encoding a Cas nuclease (e.g., Cas9). In some embodiments, the provided lipid particles contain guide RNA (gRNA), such as a single guide RNA (sgRNA).

In some embodiments, the provided lipid particles (e.g. lentiviral particles, VLPs, or gesicles) containing a Cas nuclease (e.g. Cas9) further comprise, or is further complexed with, one or more CRISPR-Cas system guide RNA(s) for targeting a desired target gene. In some embodiments, the CRISPR guide RNAs are efficiently encapsulated in the CAS-containing lipid particles. In some embodiments, the provided lipid particles (e.g. lentiviral particles, VLPs, or gesicles) further comprises, or is further complexed with a targeting nucleic acid.

In some embodiments, the heterologous protein is a recombinase. In some embodiments, the recombinase is a tyrosine recombinase. In some embodiments, the recombinase is derived from a bacteriophage. In some embodiments the recombinase is a Cre recombinase. In some embodiments, the heterologous protein is a Cre recombinase. In some embodiments, the Cre recombinase has nuclear localization signal attached to it. In some embodiments, the sequence of the heterologous protein is set forth in SEQ ID NO: 202 or 203. In some embodiments, viral genomic mRNA comprises the heterologous protein. In some embodiments, viral genomic mRNA encodes viral genes and the heterologous protein. In some embodiments, the heterologous protein is encoded in RNA which further comprises RNA encoding viral genes. In some embodiments, lentivirus vectors are used to deliver mRNA encoding the heterologous modifying protein and viral genomic mRNA. In some embodiments, lentivirus vectors comprising mRNA encoding the heterologous modifying protein and viral genomic mRNA are made by delivering DNA encoding the heterologous modifying protein and viral genomic mRNA to cells. In some embodiments, the DNA encoding the heterologous modifying protein and viral genomic mRNA comprises a sequence that exhibits at least at or about 80%, 85%, 90% or 95% sequence identity to the sequence set forth in SEQ ID NOs: 47-51. In some embodiments, the major splice donor in the viral genomic mRNA is mutated. In some embodiments, the DNA encoding the heterologous modifying protein and viral genomic mRNA with a mutated major splice donor comprises a sequence that exhibits at least at or about 80%, 85%, 90% or 95% sequence identity to the sequence set forth in SEQ ID NO: 141. In some embodiments, the DNA encoding the heterologous modifying protein and viral genomic mRNA with a mutated major splice donor comprises a sequence that exhibits at least at or about 80%, 85%, 90% or 95% sequence identity to the sequence set forth in SEQ ID NO: 200. In some embodiments, the DNA encoding the heterologous modifying protein and viral genomic mRNA encodes a sequence of amino acids that exhibits at least at or about 80%, 85%, 90% or 95% sequence identity to the sequence set forth in SEQ ID NOs: 72 or 132.

In some embodiments, the heterologous protein is encoded in RNA further comprising MS2 stem loops. In some embodiments, a DNA construct is used which encodes a lentiviral vector comprising MS2_{cp}. In some embodiments, RNA comprising a sequence encoding the heterologous protein and MS2 are capable of binding MS2_{cp}. In some embodiments, the DNA construct that drives expression of MS2_{cp} comprises a sequence that exhibits at least at or about 80%, 85%, 90% or 95% sequence identity to the sequence set forth in SEQ ID NOs: 53. In some embodiments, the DNA encoding the heterologous modifying protein and viral genomic mRNA encodes a sequence of amino acids that exhibits at least at or about 80%, 85%, 90% or 95% sequence identity to the sequence set forth in SEQ ID NOs: 73 or 133. In some embodiments, RNA comprising a sequence encoding the heterologous protein and MS2 are capable of binding MS2_{cp} expressed on gesicles. In some embodiments, gesicles are driven from over expression of a VSV-G envelope protein. In some embodiments, the DNA construct that drives expression of MS2_{cp} comprises a sequence that exhibits at least at or about 80%, 85%, 90% or 95% sequence identity to the sequence set forth in SEQ ID NOs: 54-62. In some embodiments, the DNA encoding the heterologous modifying protein and viral genomic mRNA encodes a sequence of amino acids that exhibits at least at or about 80%, 85%, 90% or 95% sequence identity to the sequence set forth in SEQ ID NOs: 74 or 134. In some embodiments, the RNA encoding a heterologous protein and MS2 stem loops are encoded in a DNA plasmid sequence set forth in SEQ ID NOs: 163-167. In some embodiments, the heterologous protein is encoded in RNA further comprising boxB binding sites. In some embodiments, a DNA construct is used which encodes a lentiviral vector comprising λN. In some embodiments, RNA comprising a sequence encoding the heterologous protein and boxB binding sites are capable of binding λN. In some embodiments, the RNA encoding a heterologous protein and boxB binding sites are encoded in a DNA sequence set forth in SEQ ID NOs: 168-173.

In some embodiments, the heterologous protein is fused to a protein which binds the interior of the viral particle. In some embodiments, the heterologous protein is fused to a domain which binds the interior of the viral particle. In some embodiments, the heterologous protein is fused to a reversible membrane attachment domain to bind the heterologous protein reversibly to the interior of the viral particle. In some embodiments, the heterologous protein is fused to a membrane attachment domain and is encoded in DNA comprising a sequence that exhibits at least at or about 80%, 85%, 90% or 95% sequence identity to the sequence set forth in SEQ ID NOs: 63-71. In some embodiments, the heterologous protein is fused to a membrane attachment domain and comprises a sequence of amino acids that exhibits at least at or about 80%, 85%, 90% or 95% sequence identity to the sequence set forth in SEQ ID NOs: 75 or 135.

### Fusogenic Proteins

In some embodiments, the lipid particle (e.g., a lentiviral particle) is provided as a fusosome. In some embodiments, the lipid particle comprises one or more fusogens. In some embodiments, the fusogen facilitates the fusion of the lipid particle to a membrane. In some embodiments, the membrane is a plasma cell membrane. In some embodiments, the membrane is a plasma cell membrane of a target cell.

In some embodiments, the lipid particle comprising the fusogen (also called a “fusosome” herein) integrates into a membrane (e.g., a lipid bilayer) of a target cell. In some embodiments, one or more of the fusogens described herein may be included in the lipid particle.

In some embodiments, the fusogen is a pseudotyping viral envelope protein. In some embodiments the lipid particle is pseudotyped with a viral envelope protein. In some embodiments, the viral envelope protein is a retargeted viral envelope protein. In some embodiments, the viral envelope protein is vesicular stomatitis virus G protein (VSV-G). In some embodiments, the VSV-G comprises the amino acid sequence set forth in SEQ ID NO: 189 Thus, in some embodiments, the lipid particle is pseudotyped with VSV-G. In some embodiments, the viral envelopment protein is different viral envelope protein or a functional portion thereof, or a combination of one or more other viral envelope proteins or functional portions thereof.

### A. Protein Fusogens

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen results in mixing between lipids in the lipid particle and lipids in the target cell. In some embodiments, the fusogen results in formation of one or more pores between the interior of the lipid particle and the cytosol of the target cell.

### 1. Mammalian Proteins

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi: 10.1038/nature12343), myomixer (nature.com/nature/journal/v499/n7458/full/nature12343.html, doi: 10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells (see Table 2), any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

### 2. Viral Proteins

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatatis Virus (VSV-G), Cocal virus G protein), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa california multiple NPV (AcMNPV) gp64), Baboon endogenous retrovirus envelope glycoprotein (BaEV), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of a Morbillivirus (e.g., measles virus (MeV), canine distemper virus, Cetacean morbillivirus, Peste-des-petits-ruminants virus, Phocine distemper virus, Rinderpest virus), Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof. In some embodiments, the viral fusogen is VSV-G. Sequences of VSV-G are known (e.g., UniProt No. P03522 and UniProt No. B7UCZ5).

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises α helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi: 10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi: 10.1016/j.devcel.2007.12.008).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, the fusogen is a poxviridae fusogen.

In some embodiments the fusogen is a paramyxovirus fusogen. In some embodiments, the fusogen may be an envelope glycoprotein G, H HN and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In particular embodiments, the fusogen is glycoprotein GP64 of baculovirus, glycoprotein GP64 variant E45K/T259A.

In some embodiments, the fusogen is a hemagglutinin-neuraminidase (HN) and fusion (F) proteins (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry to cells via the F protein. In some embodiments, the fusogen is a F and/or HN protein from the murine parainfluenza virus type 1 (See e.g., U.S. Pat. No. 10,704,061).

In some embodiments the fusogen is a paramyxovirus fusogen. In some embodiments, the fusogen may be or an envelope glycoprotein G, H and/or an F protein of the Paramyxoviridae family. In some embodiments the fusogen contains a Nipah virus protein F, a measles virus F protein, a canine distemper virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

d. G Proteins

In some embodiments the G protein is a Paramyxovirus (e.g., Morbillivirus or Henipavirus) G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein, a Langya Henipavirus G protein, or a biologically active portion thereof. A non-limiting list of exemplary G proteins is shown in Table 1.

The attachment G proteins are type II transmembrane glycoproteins containing an N-terminal cytoplasmic tail (e.g. corresponding to amino acids 1-49 of SEQ ID NO:1), a transmembrane domain (e.g. corresponding to amino acids 50-70 of SEQ ID NO:1, and an extracellular domain containing an extracellular stalk (e.g. corresponding to amino acids 71-187 of SEQ ID NO:1), and a globular head (corresponding to amino acids 188-602 of SEQ ID NO:1). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors ephrin B2 and ephrin B3, but is dispensable for membrane fusion (Brandel-Tretheway et al. Journal of Virology. 2019, 93 (13) e00577-19).

In particular embodiments herein, tropism of the G protein is modified. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described below, a re-targeted lipid particle can contain heterologous proteins from different species.

TABLE 1
Exemplary Henipavirus G Proteins

| | | | SEQ ID NO |
|---|---|---|---|
| | | | (without N- |
| | | SEQ | terminal |
| Viral G Protein | Sequence | ID NO | methionine) |
| Hendra Virus G | MMADSKLVSLNNNLSGKIKDQGKVIKNYYGTM | 2 | 3 |
| Protein | DIKKINDGLLDSKILGAFNTVIALLGSIIIIVMNIMII | | |
| | QNYTRTTDNQALIKESLQSVQQQIKALTDKIGTEI | | |
| | GPKVSLIDTSSTITIPANIGLLGSKISQSTSSINENV | | |
| | NDKCKFTLPPLKIHECNISCPNPLPFREYRPISQGV | | |
| | SDLVGLPNQICLQKTTSTILKPRLISYTLPINTREG | | |
| | VCITDPLLAVDNGFFAYSHLEKIGSCTRGIAKQRII | | |
| | GVGEVLDRGDKVPSMFMTNVWTPPNPSTIHHCS | | |
| | STYHEDFYYTLCAVSHVGDPILNSTSWTESLSLIR | | |
| | LAVRPKSDSGDYNQKYIAITKVERGKYDKVMPY | | |
| | GPSGIKQGDTLYFPAVGFLPRTEFQYNDSNCPIIH | | |
| | CKYSKAENCRLSMGVNSKSHYILRSGLLKYNLSL | | |
| | GGDIILQFIEIADNRLTIGSPSKIYNSLGQPVFYQAS | | |
| | YSWDTMIKLGDVDTVDPLRVQWRNNSVISRPGQ | | |
| | SQCPRFNVCPEVCWEGTYNDAFLIDRLNWVSAG | | |
| | VYLNSNQTAENPVFAVFKDNEILYQVPLAEDDTN | | |
| | AQKTITDCFLLENVIWCISLVEIYDTGDSVIRPKLF | | |
| | AVKIPAQCSES | | |
| Nipah Virus G | MPAENKKVRFENTTSDKGKIPSKVIKSYYGTMDI | 4 | 5 |
| Protein | KKINEGLLDSKILSAFNTVIALLGSIVIIVMNIMIIQ | | |
| | NYTRSTDNQA VIKDALQGIQQQIKGLADKIGTEIG | | |
| | PKVSLIDTSSTITIPANIGLLGSKISQSTASINENVN | | |
| | EKCKFTLPPLKIHECNISCPNPLPFREYRPQTEGVS | | |
| | NLVGLPNNICLQKTSNQILKPKLISYTLPVVGQSG | | |
| | TCITDPLLAMDEGYFAYSHLERIGSCSRGVSKQRI | | |
| | IGVGEVLDRGDEVPSLFMTNVWTPPNPNTVYHC | | |
| | SAVYNNEFYYVLCAVSTVGDPILNSTYWSGSLM | | |
| | MTRLAVKPKSNGGGYNQHQLALRSIEKGRYDKV | | |
| | MPYGPSGIKQGDTLYFPAVGFLVRTEFKYNDSNC | | |
| | PITKCQYSKPENCRLSMGIRPNSHYILRSGLLKYN | | |
| | LSDGENPKVVFIEISDQRLSIGSPSKIYDSLGQPVF | | |
| | YQASFSWDTMIKFGDVLTVNPLVVNWRNNTVIS | | |
| | RPGQSQCPRFNTCPEICWEGVYNDAFLIDRINWIS | | |
| | AGVFLDSNQTAENPVFTVFKDNEILYRAQLASED | | |
| | TNAQKTITNCFLLKNKIWCISLVEIYDTGDNVIRP | | |
| | KLFAVKIPEQCT | | |
| Cedar Virus G | MLSQLQKNYLDNSNQQGDKMNNPDKKLSVNFN | 6 | 7 |
| Protein | PLELDKGQKDLNKSYYVKNKNYNVSNLLNESLH | | |
| | DIKFCIYCIFSLLIIITIINIITISIVITRLKVHEENNGM | | |
| | ESPNLQSIQDSLSSLTNMINTEITPRIGILVTATSVT | | |
| | LSSSINYVGTKTNQLVNELKDYITKSCGFKVPELK | | |
| | LHECNISCADPKISKSAMYSTNAYAELAGPPKIFC | | |
| | KSVSKDPDFRLKQIDYVIPVQQDRSICMNNPLLDI | | |
| | SDGFFTYIHYEGINSCKKSDSFKVLLSHGEIVDRG | | |
| | DYRPSLYLLSSHYHPYSMQVINCVPVTCNQSSFV | | |
| | FCHISNNTKTLDNSDYSSDEYYITYFNGIDRPKTK | | |
| | KIPINNMTADNRYIHFTFSGGGGVCLGEEFIIPVTT | | |
| | VINTDVFTHDYCESFNCSVQTGKSLKEICSESLRS | | |
| | PTNSSRYNLNGIMIISQNNMTDFKIQLNGITYNKL | | |
| | SFGSPGRLSKTLGQVLYYQSSMSWDTYLKAGFV | | |
| | EKWKPFTPNWMNNTVISRPNQGNCPRYHKCPEI | | |
| | CYGGTYNDIAPLDLGKDMYVSVILDSDQLAENPE | | |
| | ITVFNSTTILYKERVSKDELNTRSTTTSCFLFLDEP | | |
| | WCISVLETNRFNGKSIRPEIYSYKIPKYC | | |
| Bat | MPQKTVEFINMNSPLERGVSTLSDKKTLNQSKIT | 8 | 9 |
| Paramyxovirus | KQGYFGLGSHSERNWKKQKNQNDHYMTVSTMI | | |
| G Protein, | LEILVVLGIMFNLIVLTMVYYQNDNINQRMAELT | | |
| Eid_hel/GH- | SNITVLNLNLNQLTNKIQREIIPRITLIDTATTITIPS | | |
| M74a/GHA/2009 | AITYILATLTTRISELLPSINQKCEFKTPTLVLNDC | | |
| | RINCTPPLNPSDGVKMSSLATNLVAHGPSPCRNFS | | |
| | SVPTIYYYRIPGLYNRTALDERCILNPRLTISSTKF | | |
| | AYVHSEYDKNCTRGFKYYELMTFGEILEGPEKEP | | |
| | RMFSRSFYSPTNAVNYHSCTPIVTVNEGYFLCLE | | |
| | CTSSDPLYKANLSNSTFHLVILRHNKDEKIVSMPS | | |
| | FNLSTDQEYVQIIPAEGGGTAESGNLYFPCIGRLL | | |
| | HKRVTHPLCKKSNCSRTDDESCLKSYYNQGSPQ | | |
| | HQVVNCLIRIRNAQRDNPTWDVITVDLTNTYPGS | | |
| | RSRIFGSFSKPMLYQSSVSWHTLLQVAEITDLDK | | |
| | YQLDWLDTPYISRPGGSECPFGNYCPTVCWEGTY | | |
| | NDVYSLTPNNDLFVTVYLKSEQVAENPYFAIFSR | | |
| | DQILKEFPLDAWISSARTTTISCFMFNNEIWCIAAL | | |
| | EITRLNDDIIRPIYYSFWLPTDCRTPYPHTGKMTR | | |
| | VPLRSTYNY | | |
| Mojiang virus, | MATNRDNTITSAEVSQEDKVKKYYGVETAEKVA | 10 | 11 |
| Tongguan 1 G | DSISGNKVFILMNTLLILTGAIITITLNITNLTAAKS | | |
| Protein | QQNMLKIIQDDVNAKLEMFVNLDQLVKGEIKPK | | |
| | VSLINTAVSVSIPGQISNLQTKFLQKYVYLEESITK | | |
| | QCTCNPLSGIFPTSGPTYPPTDKPDDDTTDDDKV | | |
| | DTTIKPIEYPKPDGCNRTGDHFTMEPGANFYTVP | | |
| | NLGPASSNSDECYTNPSFSIGSSIYMFSQEIRKTDC | | |
| | TAGEILSIQIVLGRIVDKGQQGPQASPLLVWAVPN | | |
| | PKIINSCAVAAGDEMGWVLCSVTLTAASGEPIPH | | |
| | MFDGFWLYKLEPDTEVVSYRITGYAYLLDKQYD | | |
| | SVFIGKGGGIQKGNDLYFQMYGLSRNRQSFKALC | | |
| | EHGSCLGTGGGGYQVLCDRAVMSFGSEESLITNA | | |
| | YLKVNDLASGKPVIIGQTFPPSDSYKGSNGRMYTI | | |
| | GDKYGLYLAPSSWNRYLRFGITPDISVRSTTWLK | | |
| | SQDPIMKILSTCTNTDRDMCPEICNTRGYQDIFPL | | |
| | SEDSEYYTYIGITPNNGGTKNFVAVRDSDGHIASI | | |
| | DILQNYYSITSATISCFMYKDEIWCIAITEGKKQK | | |
| | DNPQRIYAHSYKIRQMCYNMKSATVTVGNAKNI | | |
| | TIRRY | | |

In some embodiments, the G protein has a sequence set forth in any of SEQ ID NOs: 1-11 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOs: 1, 2, 3, 4, 5, 6, 7, 8, 9, 10 or 11. In some embodiments, the G protein has a sequence set forth in SEQ ID NO:1 or is a functionally active variant or biologically active portion thereof that has a sequence that is at least at or about 80%, at least at or about 90%, at least at or about 95%, or at least at or about 99% identical to SEQ ID NO:1. In some embodiments, the G protein has a sequence set forth in SEQ ID NO:4 or is a functionally active variant or biologically active portion thereof that has a sequence that is at least at or about 80%, at least at or about 90%, at least at or about 95%, or at least at or about 99% identical to SEQ ID NO:4. In some embodiments, the G protein has a sequence set forth in SEQ ID NO:5 or is a functionally active variant or biologically active portion thereof that has a sequence that is at least at or about 80%, at least at or about 90%, at least at or about 95%, or at least at or about 99% identical to SEQ ID NO:5.

In particular embodiments, the G protein or functionally active variant or biologically active portion is a protein that retains fusogenic activity in conjunction with a Henipavirus F protein, e.g. NiV—F or HeV-F. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

In particular embodiments, the G protein has the sequence of amino acids set forth in SEQ ID NO:1, SEQ ID NO:2, SEQ ID NO:3, SEQ ID NO:4, SEQ ID NO:5, SEQ ID NO:6, SEQ ID NO: 7, SEQ ID NO:8, SEQ ID NO:9, SEQ ID NO: 10 or SEQ ID NO: 11 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 1. SEQ ID NO:2, SEQ ID NO:3, SEQ ID NO:4, SEQ ID NO:5, SEQ ID NO:6, SEQ ID NO:7, SEQ ID NO: 8. SEQ ID NO:9, SEQ ID NO: 10 or SEQ ID NO:11 and retains fusogenic activity in conjunction with a Henipavirus F protein (e.g., NiV—F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10 or SEQ ID NO: 11 and retains fusogenic activity in conjunction with a Henipavirus F protein (e.g., NiV—F or HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NO: 1. SEQ ID NO:2, SEQ ID NO: 3. SEQ ID NO:4, SEQ ID NO:5, SEQ ID NO:6, SEQ ID NO:7, SEQ ID NO:8, SEQ ID NO:9, SEQ ID NO: 10 or SEQ ID NO: 11 such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NO:1, SEQ ID NO:2, SEQ ID NO:3, SEQ ID NO:4, SEQ ID NO:5, SEQ ID NO:6, SEQ ID NO: 7, SEQ ID NO:8, SEQ ID NO:9, SEQ ID NO: 10 or SEQ ID NO:11.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NO:1, SEQ ID NO:2, SEQ ID NO:3, SEQ ID NO:4, SEQ ID NO: 5, SEQ ID NO:6, SEQ ID NO:7, SEQ ID NO:8, SEQ ID NO:9, SEQ ID NO: 10 or SEQ ID NO: 11. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO: 1, SEQ ID NO:4 or SEQ ID NO:5, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:1, SEQ ID NO:4 or SEQ ID NO:5. In some embodiments, the G protein is a NiV-G protein that has the sequence set forth in SEQ ID NO:1, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO: 1. In some embodiments, the G protein is a NiV-G protein that has the sequence set forth in SEQ ID NO: 1. In some embodiments, the G protein is a NiV-G protein that has the sequence set forth in SEQ ID NO: 4, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:4. In some embodiments, the G protein is a NiV-G protein that has the sequence set forth in SEQ ID NO:4. In some embodiments, the G protein is a NiV-G protein that has the sequence set forth in SEQ ID NO:5, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:5. In some embodiments, the G protein is a NiV-G protein that has the sequence set forth in SEQ ID NO:5.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:1, SEQ ID NO:4 or SEQ ID NO:5), up to 6 contiguous amino acid residues at or near the N-terminus of the wild-type NiV-G protein (SEQ ID NO:1. SEQ ID NO:4 or SEQ ID NO:5), up to 7 contiguous amino acid residues at or near the N-terminus of the wild-type NiV-G protein (SEQ ID NO:1. SEQ ID NO:4 or SEQ ID NO:5), up to 8 contiguous amino acid residues at or near the N-terminus of the wild-type NiV-G protein (SEQ ID NO:1. SEQ ID NO:4 or SEQ ID NO:5), up to 9 contiguous amino acid residues at or near the N-terminus of the wild-type NiV-G protein (SEQ ID NO: 1. SEQ ID NO:4 or SEQ ID NO:5) up to 10 contiguous amino acid residues at or near the N-terminus of the wild-type NiV-G protein (SEQ ID NO: 1. SEQ ID NO:4 or SEQ ID NO:5), up to 11 contiguous amino acid residues at or near the N-terminus of the wild-type NiV-G protein (SEQ ID NO: 1. SEQ ID NO:4 or SEQ ID NO:5), up to 12 contiguous amino acid residues at or near the N-terminus of the wild-type NiV-G protein SEQ ID NO:1, SEQ ID NO:4, or SEQ ID NO:5), up to 13 contiguous amino acid residues at or near the N-terminus of the wild-type NiV-G protein (SEQ ID NO: 1. SEQ ID NO:4 or SEQ ID NO:5), up to 14 contiguous amino acid residues at or near the N-terminus of the wild-type NiV-G protein (SEQ ID NO: 1. SEQ ID NO:4 or SEQ ID NO:5), up to 15 contiguous amino acid residues at or near the N-terminus of the wild-type NiV-G protein (SEQ ID NO: 1. SEQ ID NO:4 or SEQ ID NO:5) up to 16 contiguous amino acid residues at or near the N-terminus of the wild-type NiV-G protein (SEQ ID NO:1. SEQ ID NO:4 or SEQ ID NO:5), up to 17 contiguous amino acid residues at or near the N-terminus of the wild-type NiV-G protein (SEQ ID NO: 1. SEQ ID NO:4 or SEQ ID NO:5), up to 18 contiguous amino acid residues at or near the N-terminus of the wild-type NiV-G protein (SEQ ID NO: 1. SEQ ID NO:4 or SEQ ID NO:5), up to 19 contiguous amino acid residues at or near the N-terminus of the wild-type NiV-G protein (SEQ ID NO: 1. SEQ ID NO:4 or SEQ ID NO:5), up to 20 contiguous amino acid residues at or near the N-terminus of the wild-type NiV-G protein (SEQ ID NO:1. SEQ ID NO:4 or SEQ ID NO:5), up to 21 contiguous amino acid residues at or near the N-terminus of the wild-type NiV-G protein SEQ ID NO: 1. SEQ ID NO:4 or SEQ ID NO:5), up to 22 contiguous amino acid residues at or near the N-terminus of the wild-type NiV-G protein (SEQ ID NO:1. SEQ ID NO:4 or SEQ ID NO:5), up to 23 contiguous amino acid residues at or near the N-terminus of the wild-type NiV-G protein (SEQ ID NO: 1. SEQ ID NO:4 or SEQ ID NO:5), up to 24 contiguous amino acid residues at or near the N-terminus of the wild-type NiV-G protein (SEQ ID NO:1. SEQ ID NO:4 or SEQ ID NO:5), up to 25 contiguous amino acid residues at or near the N-terminus of the wild-type NiV-G protein (SEQ ID NO: 1. SEQ ID NO:4 or SEQ ID NO:5), up to 26 contiguous amino acid residues at or near the N-terminus of the wild-type NiV-G protein (SEQ ID NO:1. SEQ ID NO:4 or SEQ ID NO:5), up to 27 contiguous amino acid residues at or near the N-terminus of the wild-type NiV-G protein (SEQ ID NO: 1. SEQ ID NO:4 or SEQ ID NO:5), up to 28 contiguous amino acid residues at or near the N-terminus of the wild-type NiV-G protein (SEQ ID NO:1. SEQ ID NO:4 or SEQ ID NO:5), up to 29 contiguous amino acid residues at or near the N-terminus of the wild-type NiV-G protein (SEQ ID NO: 1. SEQ ID NO:4 or SEQ ID NO:5), up to 30 contiguous amino acid residues at or near the N-terminus of the wild-type NiV-G protein (SEQ ID NO:1. SEQ ID NO:4 or SEQ ID NO:5), up to 31 contiguous amino acid residues at or near the N-terminus of the wild-type NiV-G protein (SEQ ID NO: 1, SEQ ID NO:4 or SEQ ID NO:5), up to 32 contiguous amino acid residues at or near the N-terminus of the wild-type NiV-G protein (SEQ ID NO:1. SEQ ID NO:4 or SEQ ID NO:5), up to 33 contiguous amino acid residues at or near the N-terminus of the wild-type NiV-G protein (SEQ ID NO: 1, SEQ ID NO:4 or SEQ ID NO:5), up to 34 contiguous amino acid residues at or near the N-terminus of the wild-type NiV-G protein (SEQ ID NO: 1. SEQ ID NO:4 or SEQ ID NO:5), up to 35 contiguous amino acid residues at or near the N-terminus of the wild-type NiV-G protein (SEQ ID NO: 1. SEQ ID NO:4 or SEQ ID NO:5), up to 36 contiguous amino acid residues at or near the N-terminus of the wild-type NiV-G protein (SEQ ID NO:1, SEQ ID NO:4 or SEQ ID NO:5), up to 37 contiguous amino acid residues at or near the N-terminus of the wild-type NiV-G protein (SEQ ID NO: 1, SEQ ID NO:4 or SEQ ID NO:5) up to 38 contiguous amino acid residues at or near the N-terminus of the wild-type NiV-G protein (SEQ ID NO: 1. SEQ ID NO:4 or SEQ ID NO:5), up to 39 contiguous amino acid residues at or near the N-terminus of the wild-type NiV-G protein (SEQ ID NO: 1, SEQ ID NO:4 or SEQ ID NO:5), up to 40 contiguous amino acid residues at or near the N-terminus of the wild-type NiV-G protein (SEQ ID NO:1. SEQ ID NO:4 or SEQ ID NO:5), up to 41 contiguous amino acid residues at or near the N-terminus of the wild-type NiV-G protein (SEQ ID NO: 1, SEQ ID NO:4 or SEQ ID NO:5), up to 42 contiguous amino acid residues at or near the N-terminus of the wild-type NiV-G protein (SEQ ID NO:1. SEQ ID NO:4 or SEQ ID NO:5), up to 43 contiguous amino acid residues at or near the N-terminus of the wild-type NiV-G protein (SEQ ID NO: 1. SEQ ID NO:4 or SEQ ID NO:5), up to 44 contiguous amino acid residues at or near the N-terminus of the wild-type NiV-G protein (SEQ ID NO:1. SEQ ID NO:4 or SEQ ID NO:5), or up to 45 contiguous amino acid residues at or near the N-terminus of the wild-type NiV-G protein (SEQ ID NO: 1, SEQ ID NO:4 or SEQ ID NO:5).

In some embodiments, the mutant NiV-G protein is truncated and lacks 5 contiguous amino acid residues at or near the N-terminus of the wild-type NiV-G protein (SEQ ID NO:1, SEQ ID NO: 4 or SEQ ID NO:5). In some embodiments, the mutant NiV-G protein comprises the amino acid sequence set forth in SEQ ID NO: 12. In some embodiments, the mutant NiV-G protein is truncated and lacks 10 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:44. In some embodiments, the mutant NiV-G protein is truncated and lacks 15 contiguous amino acid residues at or near the N-terminus of the wild-type NiV-G protein (SEQ ID NO:1, SEQ ID NO:4 or SEQ ID NO: 5). In some embodiments, the mutant NiV-G protein comprises the amino acid sequence set forth in SEQ ID NO:45. In some embodiments, the mutant NiV-G protein is truncated and lacks 20 contiguous amino acid residues at or near the N-terminus of the wild-type NiV-G protein (SEQ ID NO: 1, SEQ ID NO:4 or SEQ ID NO:5). In some embodiments, the mutant NiV-G protein comprises the amino acid sequence set forth in SEQ ID NO: 13. In some embodiments, the mutant NiV-G protein is truncated and lacks 25 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO: 14. In some embodiments, the mutant NiV-G protein is truncated and lacks 30 contiguous amino acid residues at or near the N-terminus of the wild-type NiV-G protein (SEQ ID NO:1, SEQ ID NO:4 or SEQ ID NO: 5). In some embodiments, the mutant NiV-G protein comprises the amino acid sequence set forth in SEQ ID NO:43. In some embodiments, the mutant NiV-G protein is truncated and lacks 34 contiguous amino acid residues at or near the N-terminus of the wild-type NiV-G protein (SEQ ID NO: 1, SEQ ID NO:4 or SEQ ID NO:5). In some embodiments, the mutant NiV-G protein comprises the amino acid sequence set forth in SEQ ID NO:42.

In some embodiments, the NiV-G protein is a biologically active portion that does not contain a cytoplasmic domain. In some embodiments, the NiV-G protein without the cytoplasmic domain is encoded by SEQ ID NO:22.

In some embodiments, the mutant NiV-G protein comprises a sequence set forth in any of SEQ ID NOS: 12-14, 17, 18 and 22, or 42-45 or is a functional variant thereof that has an amino acid sequence having at least at or 80%, at least at or about 81%, at least at or about 82%, at least at or about 83%, at or about 84%, at least at or about 85%, at least at or about 86%, or at least at or about 87%, at least at or about 88%, or at least at or about 89%, about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOS: 12-14, 17, 18 and 22 or 42-45.

In some embodiments, the mutant NiV-G protein has a 5 amino acid truncation at or near the N-terminus of the wild-type NiV-G protein (SEQ ID NO: 1, SEQ ID NO:4 or SEQ ID NO:5), such as set forth in SEQ ID NO: 12 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 12 or such as set forth in SEQ ID NO: 17 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 17. In some embodiments, the mutant NiV-G protein has a 10 amino acid truncation at or near the N-terminus of the wild-type NiV-G protein (SEQ ID NO:1, SEQ ID NO:4 or SEQ ID NO:5), such as set forth in SEQ ID NO:44 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:44. In some embodiments, the mutant NiV-G protein has a 20 amino acid truncation at or near the N-terminus of the wild-type NiV-G protein (SEQ ID NO:1, SEQ ID NO:4 or SEQ ID NO:5), such as set forth in SEQ ID NO: 13 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 13. In some embodiments, the mutant NiV-G protein has a 25 amino acid truncation at or near the N-terminus of the wild-type NiV-G protein (SEQ ID NO: 1, SEQ ID NO:4 or SEQ ID NO:5), such as set forth in SEQ ID NO: 14 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14. In some embodiments, the mutant NiV-G protein has a 33 amino acid truncation at or near the N-terminus of the wild-type NiV-G protein (SEQ ID NO:1, SEQ ID NO:4 or SEQ ID NO:5), such as set forth in SEQ ID NO: 17 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 17. In some embodiments, the mutant NiV-G protein has a 34 amino acid truncation at or near the N-terminus of the wild-type NiV-G protein (SEQ ID NO:1, SEQ ID NO:4 or SEQ ID NO:5), such as set forth in SEQ ID NO: 18 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 18. In some embodiments, the mutant NiV-G protein has a 48 amino acid truncation at or near the N-terminus of the wild-type NiV-G protein (SEQ ID NO:1, SEQ ID NO:4 or SEQ ID NO:5), such as set forth in SEQ ID NO:22 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22.

In some embodiments, the mutant NiV-G protein has a 15 amino acid truncation at or near the N-terminus of the wild-type NiV-G protein (SEQ ID NO:1, SEQ ID NO:4, or SEQ ID NO:5), such as set forth in SEQ ID NO:45 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45.

In some embodiments, the mutant NiV-G protein has a 20 amino acid truncation at or near the N-terminus of the wild-type NiV-G protein (SEQ ID NO:1. SEQ ID NO:4, or SEQ ID NO:5), such as set forth in SEQ ID NO:13 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13.

In some embodiments, the mutant NiV-G protein has a 25 amino acid truncation at or near the N-terminus of the wild-type NiV-G protein (SEQ ID NO:1, SEQ ID NO:4, or SEQ ID NO:5), such as set forth in SEQ ID NO: 14 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 14.

In some embodiments, the mutant NiV-G protein has a 30 amino acid truncation at or near the N-terminus of the wild-type NiV-G protein (SEQ ID NO: 1. SEQ ID NO:4, or SEQ ID NO:5), such as set forth in SEQ ID NO:43 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:43.

In some embodiments, the mutant NiV-G protein has a 34 amino acid truncation at or near the N-terminus of the wild-type NiV-G protein (SEQ ID NO: 1, SEQ ID NO:4, or SEQ ID NO:5), such as set forth in SEQ ID NO:42 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:42.

In some embodiments, the mutant NiV-G protein has a 48 amino acid truncation at or near the N-terminus of the wild-type NiV-G protein (SEQ ID NO:1. SEQ ID NO:4, or SEQ ID NO:5), such as set forth in SEQ ID NO:22 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22.

In some embodiments, the G protein is a mutant HeV-G protein that is a biologically active portion of a wild-type HeV-G. In some embodiments, the biologically active portion is an N-terminally truncated fragment.

In some embodiments, the G protein is a wild-type HeV-G protein that has the sequence set forth in SEQ ID NO:23 or 24, or is a functional variant or biologically active portion thereof that has an amino acid sequence having at least at or about 80%, at least at or about 81%, at least at or about 82%, at least at or about 83%, at least at or about 84%, at or about 85%, at least at or about 86%, at least at or about 87%, at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:23 or 24.

In some embodiments, the G protein is a mutant HeV-G protein that is a biologically active portion of a wild-type HeV-G (SEQ ID NO:23 or SEQ ID NO:24). In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:23 or 24), up to 6 contiguous amino acid residues at or near the N-terminus of the wild-type HeV-G protein (SEQ ID NO:23 or 24), up to 7 contiguous amino acid residues at or near the N-terminus of the wild-type HeV-G protein (SEQ ID NO: 23 or 24) or up to 8 contiguous amino acid residues at or near the N-terminus of the wild-type HeV-G protein (SEQ ID NO:23 or 24), up to 9 contiguous amino acid residues at or near the N-terminus of the wild-type HeV-G protein (SEQ ID NO:23 or 24), up to 10 contiguous amino acid residues at or near the N-terminus of the wild-type HeV-G protein (SEQ ID NO:23 or 24), up to 11 contiguous amino acid residues at or near the N-terminus of the wild-type HeV-G protein (SEQ ID NO: 23 or 24), up to 12 contiguous amino acid residues at or near the N-terminus of the wild-type HeV-G protein (SEQ ID NO:23 or 24), up to 13 contiguous amino acid residues at or near the N-terminus of the wild-type HeV-G protein (SEQ ID NO:23 or 24), up to 14 contiguous amino acid residues at or near the N-terminus of the wild-type HeV-G protein (SEQ ID NO:23 or 24), up to 15 contiguous amino acid residues at or near the N-terminus of the wild-type HeV-G protein (SEQ ID NO: 23 or 24), up to 16 contiguous amino acid residues at or near the N-terminus of the wild-type HeV-G protein (SEQ ID NO:23 or 24), up to 17 contiguous amino acid residues at or near the N-terminus of the wild-type HeV-G protein (SEQ ID NO:23 or 24), up to 18 contiguous amino acid residues at or near the N-terminus of the wild-type HeV-G protein (SEQ ID NO:23 or 24), up to 19 contiguous amino acid residues at or near the N-terminus of the wild-type HeV-G protein (SEQ ID NO: 23 or 24), up to 20 contiguous amino acid residues at or near the N-terminus of the wild-type HeV-G protein (SEQ ID NO:23 or 24), up to 21 contiguous amino acid residues at or near the N-terminus of the wild-type HeV-G protein (SEQ ID NO:23 or 24), up to 22 contiguous amino acid residues at or near the N-terminus of the wild-type HeV-G protein (SEQ ID NO:23 or 24), up to 23 contiguous amino acid residues at or near the N-terminus of the wild-type HeV-G protein SEQ ID NO: 23 or 24), up to 24 contiguous amino acid residues at or near the N-terminus of the wild-type HeV-G protein (SEQ ID NO:23 or 24), up to 25 contiguous amino acid residues at or near the N-terminus of the wild-type HeV-G protein (SEQ ID NO:23 or 24), up to 26 contiguous amino acid residues at or near the N-terminus of the wild-type HeV-G protein (SEQ ID NO:23 or 24), up to 27 contiguous amino acid residues at or near the N-terminus of the wild-type HeV-G protein (SEQ ID NO: 23 or 24), up to 28 contiguous amino acid residues at or near the N-terminus of the wild-type HeV-G protein (SEQ ID NO:23 or 24), up to 29 contiguous amino acid residues at or near the N-terminus of the wild-type HeV-G protein (SEQ ID NO:23 or 24), up to 30 contiguous amino acid residues at or near the N-terminus of the wild-type HeV-G protein (SEQ ID NO:23 or 24), up to 31 contiguous amino acid residues at or near the N-terminus of the wild-type HeV-G protein (SEQ ID NO: 23 or 24), up to 32 contiguous amino acid residues at or near the N-terminus of the wild-type HeV-G protein (SEQ ID NO:23 or 24), up to 33 contiguous amino acid residues at or near the N-terminus of the wild-type HeV-G protein (SEQ ID NO:23 or 24), up to 34 contiguous amino acid residues at or near the N-terminus of the wild-type HeV-G protein (SEQ ID NO:23 or 24), up to 35 contiguous amino acid residues at or near the N-terminus of the wild-type HeV-G protein (SEQ ID NO: 23 or 24), up to 36 contiguous amino acid residues at or near the N-terminus of the wild-type HeV-G protein (SEQ ID NO:23 or 24), up to 37 contiguous amino acid residues at or near the N-terminus of the wild-type HeV-G protein (SEQ ID NO:23 or 24), up to 38 contiguous amino acid residues at or near the N-terminus of the wild-type HeV-G protein (SEQ ID NO:23 or 24), up to 39 contiguous amino acid residues at or near the N-terminus of the wild-type HeV-G protein (SEQ ID NO: 23 or 24), up to 40 contiguous amino acid residues at or near the N-terminus of the wild-type HeV-G protein (SEQ ID NO:23 or 24), up to 41 contiguous amino acid residues at or near the N-terminus of the wild-type HeV-G protein (SEQ ID NO:23 or 24), up to 42 contiguous amino acid residues at or near the N-terminus of the wild-type HeV-G protein (SEQ ID NO:23 or 24), up to 43 contiguous amino acid residues at or near the N-terminus of the wild-type HeV-G protein (SEQ ID NO: 23 or 24), up to 44 contiguous amino acid residues at or near the N-terminus of the wild-type HeV-G protein (SEQ ID NO:23 or 24), or up to 45 contiguous amino acid residues at or near the N-terminus of the wild-type HeV-G protein (SEQ ID NO:23 or 24).

In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:23 or 24), such as set forth in SEQ ID NO: 25 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:25. In some embodiments, the mutant HeV-G protein lacks the N-terminal cytoplasmic domain of the wild-type HeV-G protein (SEQ ID NO:23 or 24), such as set forth in SEQ ID NO:26 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26.

In some embodiments, the G protein or the functionally active variant or biologically active portion thereof binds to Ephrin B2 or Ephrin B3. In some aspects, the G protein has the sequence of amino acids set forth in any one of SEQ ID NO:24, SEQ ID NO:23, SEQ ID NO:4, SEQ ID NO:6, SEQ ID NO: 5, SEQ ID NO:8 or SEQ ID NO: 10, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to any of SEQ ID NO:24, SEQ ID NO:23, SEQ ID NO:4, SEQ ID NO:6, SEQ ID NO:5, SEQ ID NO:8 or SEQ ID NO:10, or a functionally active variant or biologically active portion thereof, and retains binding to Ephrin B2 or B3.

In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:27, SEQ ID NO:23, SEQ ID NO:4, SEQ ID NO:6, SEQ ID NO:5, SEQ ID NO: 8 or SEQ ID NO:10, or a functionally active variant or biologically active portion thereof, and retains binding to Ephrin B2 or B3. Reference to retaining binding to Ephrin B2 or B3 includes binding that is at least or at least about 5% of the level or degree of binding of the corresponding wild-type G protein, such as set forth in SEQ ID NO:27. SEQ ID NO:23. SEQ ID NO:4. SEQ ID NO:6. SEQ ID NO: 5. SEQ ID NO:8 or SEQ ID NO:10, or a functionally active variant or biologically active portion thereof. 10% of the level or degree of binding of the corresponding wild-type G protein, such as set forth in SEQ ID NO:27. SEQ ID NO:23. SEQ ID NO:4. SEQ ID NO:6. SEQ ID NO:5. SEQ ID NO:8 or SEQ ID NO: 10, or a functionally active variant or biologically active portion thereof. 15% of the level or degree of binding of the corresponding wild-type G protein, such as set forth in SEQ ID NO:27. SEQ ID NO: 23. SEQ ID NO:4. SEQ ID NO:6. SEQ ID NO:5. SEQ ID NO:8 or SEQ ID NO: 10, or a functionally active variant or biologically active portion thereof. 20% of the level or degree of binding of the corresponding wild-type G protein, such as set forth in SEQ ID NO:27. SEQ ID NO:23. SEQ ID NO:4. SEQ ID NO:6. SEQ ID NO:5. SEQ ID NO:8 or SEQ ID NO: 10, or a functionally active variant or biologically active portion thereof. 25% of the level or degree of binding of the corresponding wild-type G protein, such as set forth in SEQ ID NO:27. SEQ ID NO:23. SEQ ID NO:4. SEQ ID NO:6. SEQ ID NO: 5. SEQ ID NO:8 or SEQ ID NO: 10, or a functionally active variant or biologically active portion. 30% of the level or degree of binding of the corresponding wild-type G protein, such as set forth in SEQ ID NO: 27. SEQ ID NO:23. SEQ ID NO:4. SEQ ID NO:6. SEQ ID NO:5. SEQ ID NO:8 or SEQ ID NO: 10, or a functionally active variant or biologically active portion thereof. 35% of the level or degree of binding of the corresponding wild-type G protein, such as set forth in SEQ ID NO:27. SEQ ID NO:23. SEQ ID NO:4. SEQ ID NO:6. SEQ ID NO:5. SEQ ID NO:8 or SEQ ID NO: 10, or a functionally active variant or biologically active portion thereof. 40% of the level or degree of binding of the corresponding wild-type G protein, such as set forth in SEQ ID NO:27. SEQ ID NO:23. SEQ ID NO:4. SEQ ID NO:6. SEQ ID NO:5. SEQ ID NO:8 or SEQ ID NO: 10, or a functionally active variant or biologically active portion thereof. 45% of the level or degree of binding of the corresponding wild-type G protein, such as set forth in SEQ ID NO:27. SEQ ID NO:23. SEQ ID NO:4. SEQ ID NO:6. SEQ ID NO:5. SEQ ID NO:8 or SEQ ID NO:10, or a functionally active variant or biologically active portion thereof. 50% of the level or degree of binding of the corresponding wild-type G protein, such as set forth in SEQ ID NO:27. SEQ ID NO: 23. SEQ ID NO:4. SEQ ID NO:6. SEQ ID NO:5. SEQ ID NO:8 or SEQ ID NO: 10, or a functionally active variant or biologically active portion thereof. 55% of the level or degree of binding of the corresponding wild-type G protein, such as set forth in SEQ ID NO:27. SEQ ID NO:23. SEQ ID NO: 4. SEQ ID NO:6. SEQ ID NO:5. SEQ ID NO:8 or SEQ ID NO: 10, or a functionally active variant or biologically active portion thereof. 60% of the level or degree of binding of the corresponding wild-type G protein, such as set forth in SEQ ID NO:27. SEQ ID NO:23, SEQ ID NO:4. SEQ ID NO:6. SEQ ID NO: 5. SEQ ID NO:8 or SEQ ID NO: 10, or a functionally active variant or biologically active portion thereof. 65% of the level or degree of binding of the corresponding wild-type G protein, such as set forth in SEQ ID NO:27. SEQ ID NO:23, SEQ ID NO:4. SEQ ID NO:6. SEQ ID NO:5. SEQ ID NO:8 or SEQ ID NO: 10, or a functionally active variant or biologically active portion thereof. 70% of the level or degree of binding of the corresponding wild-type G protein, such as set forth in SEQ ID NO:27, SEQ ID NO: 23, SEQ ID NO:4, SEQ ID NO:6, SEQ ID NO:5, SEQ ID NO:8 or SEQ ID NO:10 or a functionally active variant or biologically active portion thereof, such as at least or at least about 75% of the level or degree of binding of the corresponding wild-type G protein, such as set forth in SEQ ID NO:27, SEQ ID NO: 23, SEQ ID NO:4, SEQ ID NO:6, SEQ ID NO:5, SEQ ID NO:8 or SEQ ID NO: 10, or a functionally active variant or biologically active portion thereof, such as at least or at least about 80% of the level or degree of binding of the corresponding wild-type G protein, such as set forth in SEQ ID NO:27, SEQ ID NO: 23, SEQ ID NO:4, NO: 4, SEQ ID NO:6, SEQ ID NO:5, SEQ ID NO:8 or SEQ ID NO: 10, or a functionally active variant or biologically active portion thereof, such as at least or at least about 85% of the level or degree of binding of the corresponding wild-type G protein, such as set forth in SEQ ID NO: 27, SEQ ID NO:23, SEQ ID NO:4, SEQ ID NO:6, SEQ ID NO:5, SEQ ID NO:8 or SEQ ID NO: 10, or a functionally active variant or biologically active portion thereof, such as at least or at least about 90% of the level or degree of binding of the corresponding wild-type G protein, such as set forth in SEQ ID NO: 27, SEQ ID NO:23, SEQ ID NO:4, SEQ ID NO:6, SEQ ID NO:5, SEQ ID NO:8 or SEQ ID NO: 10, or a functionally active variant or biologically active portion thereof, or such as at least or at least about 95% of the level or degree of binding of the corresponding wild-type protein, such as set forth in SEQ ID NO:27, SEQ ID NO:23, SEQ ID NO:4, SEQ ID NO:6, SEQ ID NO:5, SEQ ID NO:8 or SEQ ID NO: 10, or a functionally active variant or biologically active portion thereof. In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:4, SEQ ID NO:5 or SEQ ID NO:27, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4, SEQ ID NO: 5 or SEQ ID NO:27 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type NiV-G, such as set forth in SEQ ID NO:4, SEQ ID NO:5 or SEQ ID NO:27, 10% of the level or degree of binding of the corresponding wild-type NiV-G, such as set forth in SEQ ID NO: 4, SEQ ID NO:5 or SEQ ID NO:27, 15% of the level or degree of binding of the corresponding wild-type NiV-G, such as set forth in SEQ ID NO:4, SEQ ID NO:5 or SEQ ID NO:27, 20% of the level or degree of binding of the corresponding wild-type NiV-G, such as set forth in SEQ ID NO:4, SEQ ID NO: 5 or SEQ ID NO:27, 25% of the level or degree of binding of the corresponding wild-type NiV-G, such as set forth in SEQ ID NO:4, SEQ ID NO:5 or SEQ ID NO:27, 30% of the level or degree of binding of the corresponding wild-type NiV-G, such as set forth in SEQ ID NO:4, SEQ ID NO:5 or SEQ ID NO: 27, 35% of the level or degree of binding of the corresponding wild-type NiV-G, such as set forth in SEQ ID NO:4, SEQ ID NO:5 or SEQ ID NO:27, 40% of the level or degree of binding of the corresponding wild-type NiV-G, such as set forth in SEQ ID NO:4, SEQ ID NO:5 or SEQ ID NO:27, 45% of the level or degree of binding of the corresponding wild-type NiV-G, such as set forth in SEQ ID NO: 4, SEQ ID NO:5 or SEQ ID NO:27 50% of the level or degree of binding of the corresponding wild-type NiV-G, such as set forth in SEQ ID NO:4, SEQ ID NO:5 or SEQ ID NO:27, 55% of the level or degree of binding of the corresponding wild-type NiV-G, such as set forth in SEQ ID NO:4, SEQ ID NO: 5 or SEQ ID NO:27, 60% of the level or degree of binding of the corresponding wild-type NiV-G, such as set forth in SEQ ID NO:4, SEQ ID NO:5 or SEQ ID NO:27, 65% of the level or degree of binding of the corresponding wild-type NiV-G, such as set forth in SEQ ID NO:4, SEQ ID NO:5 or SEQ ID NO: 27, 70% of the level or degree of binding of the corresponding wild-type NiV-G, such as set forth in SEQ ID NO:4, SEQ ID NO:5 or SEQ ID NO:27, such as at least or at least about 75% of the level or degree of binding of the corresponding wild-type NiV-G, such as set forth in SEQ ID NO:4, SEQ ID NO: 5 or SEQ ID NO:27, such as at least or at least about 80% of the level or degree of binding of the corresponding wild-type NIV-G, such as set forth in SEQ ID NO:4, SEQ ID NO:5 or SEQ ID NO:27, such as at least or at least about 85% of the level or degree of binding of the corresponding wild-type NiV-G, such as set forth in SEQ ID NO:4, SEQ ID NO:5 or SEQ ID NO:27, such as at least or at least about 90% of the level or degree of binding of the corresponding wild-type NiV-G, such as set forth in SEQ ID NO:4, SEQ ID NO:5 or SEQ ID NO:27, or such as at least or at least about 95% of the level or degree of binding of the corresponding wild-type NiV-G, such as set forth in SEQ ID NO:4, SEQ ID NO: 5 or SEQ ID NO:27.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:23 or 24, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 23 or 24 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type HeV-G, such as set forth in SEQ ID NO:23 or 24, 10% of the level or degree of binding of the corresponding wild-type HeV-G, such as set forth in SEQ ID NO:23 or 24, 15% of the level or degree of binding of the corresponding wild-type HeV-G, such as set forth in SEQ ID NO:23 or 24, 20% of the level or degree of binding of the corresponding wild-type HeV-G, such as set forth in SEQ ID NO:23 or 24, 25% of the level or degree of binding of the corresponding wild-type HeV-G, such as set forth in SEQ ID NO:23 or 24, 30% of the level or degree of binding of the corresponding wild-type HeV-G, such as set forth in SEQ ID NO:23 or 24, 35% of the level or degree of binding of the corresponding wild-type HeV-G, such as set forth in SEQ ID NO:23 or 24, 40% of the level or degree of binding of the corresponding wild-type HeV-G, such as set forth in SEQ ID NO:23 or 24, 45% of the level or degree of binding of the corresponding wild-type HeV-G, such as set forth in SEQ ID NO:23 or 24, 50% of the level or degree of binding of the corresponding wild-type HeV-G, such as set forth in SEQ ID NO:23 or 24, 55% of the level or degree of binding of the corresponding wild-type HeV-G, such as set forth in SEQ ID NO:23 or 24, 60% of the level or degree of binding of the corresponding wild-type HeV-G, such as set forth in SEQ ID NO:23 or 24, 65% of the level or degree of binding of the corresponding wild-type HeV-G, such as set forth in SEQ ID NO:23 or 24, 70% of the level or degree of binding of the corresponding wild-type HeV-G, such as set forth in SEQ ID NO:23 or 24, such as at least or at least about 75% of the level or degree of binding of the corresponding wild-type HeV-G, such as set forth in SEQ ID NO:23 or 24, such as at least or at least about 80% of the level or degree of binding of the corresponding wild-type NIV-G, such as set forth in SEQ ID NO:23 or 24, such as at least or at least about 85% of the level or degree of binding of the corresponding wild-type HeV-G, such as set forth in SEQ ID NO:23 or 24, such as at least or at least about 90% of the level or degree of binding of the corresponding wild-type HeV-G, such as set forth in SEQ ID NO:23 or 24, or such as at least or at least about 95% of the level or degree of binding of the corresponding wild-type HeV-G, such as set forth in SEQ ID NO:23 or 24.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:4.

In some embodiments, the G protein is a mutant G protein. In some embodiments, the G protein is a mutant G protein containing one or more amino acid substitutions selected from the group consisting of E501A, W504A, Q530A and E533A with reference to numbering set forth in SEQ ID NO:4. In some embodiments, the G protein is a mutant G protein that contains one or more amino acid substitutions elected from the group consisting of E501A, W504A, Q530A and E533A with reference to SEQ ID NO:4 and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO:4), 6 contiguous amino acid residues at or near the N-terminus of the wild-type NiV-G protein (SEQ ID NO:4), 7 contiguous amino acid residues at or near the N-terminus of the wild-type NiV-G protein (SEQ ID NO:4), 8 contiguous amino acid residues at or near the N-terminus of the wild-type NiV-G protein (SEQ ID NO:4), 9 contiguous amino acid residues at or near the N-terminus of the wild-type NiV-G protein (SEQ ID NO:4), up to 10 contiguous amino acid residues at or near the N-terminus of the wild-type NiV-G protein (SEQ ID NO: 4), 11 contiguous amino acid residues at or near the N-terminus of the wild-type NiV-G protein (SEQ ID NO:4), 12 contiguous amino acid residues at or near the N-terminus of the wild-type NiV-G protein (SEQ ID NO:4), 13 contiguous amino acid residues at or near the N-terminus of the wild-type NiV-G protein (SEQ ID NO:4), 14 contiguous amino acid residues at or near the N-terminus of the wild-type NiV-G protein (SEQ ID NO:4), up to 15 contiguous amino acid residues at or near the N-terminus of the wild-type NiV-G protein (SEQ ID NO:4), 16 contiguous amino acid residues at or near the N-terminus of the wild-type NiV-G protein (SEQ ID NO:4), 17 contiguous amino acid residues at or near the N-terminus of the wild-type NiV-G protein (SEQ ID NO:4), 18 contiguous amino acid residues at or near the N-terminus of the wild-type NiV-G protein (SEQ ID NO: 4), 19 contiguous amino acid residues at or near the N-terminus of the wild-type NiV-G protein (SEQ ID NO:4), up to 20 contiguous amino acid residues at or near the N-terminus of the wild-type NiV-G protein (SEQ ID NO:4), 21 contiguous amino acid residues at or near the N-terminus of the wild-type NiV-G protein (SEQ ID NO:4) 22 contiguous amino acid residues at or near the N-terminus of the wild-type NiV-G protein (SEQ ID NO:4), 23 contiguous amino acid residues at or near the N-terminus of the wild-type NiV-G protein (SEQ ID NO:4), 24 contiguous amino acid residues at or near the N-terminus of the wild-type NiV-G protein (SEQ ID NO:4), up to 25 contiguous amino acid residues at or near the N-terminus of the wild-type NiV-G protein (SEQ ID NO: 4), 26 contiguous amino acid residues at or near the N-terminus of the wild-type NiV-G protein (SEQ ID NO:4), 27 contiguous amino acid residues at or near the N-terminus of the wild-type NiV-G protein (SEQ ID NO:4), 28 contiguous amino acid residues at or near the N-terminus of the wild-type NiV-G protein (SEQ ID NO:4), 29 contiguous amino acid residues at or near the N-terminus of the wild-type NiV-G protein (SEQ ID NO:4), up to 30 contiguous amino acid residues at or near the N-terminus of the wild-type NiV-G protein (SEQ ID NO: 4), up to 31 contiguous amino acid residues at or near the N-terminus of the wild-type NiV-G protein (SEQ ID NO:4), 32 contiguous amino acid residues at or near the N-terminus of the wild-type NiV-G protein (SEQ ID NO:4), 33 contiguous amino acid residues at or near the N-terminus of the wild-type NiV-G protein (SEQ ID NO: 4) 34 contiguous amino acid residues at or near the N-terminus of the wild-type NiV-G protein (SEQ ID NO:4), 35 contiguous amino acid residues at or near the N-terminus of the wild-type NiV-G protein (SEQ ID NO:4) up to 36 contiguous amino acid residues at or near the N-terminus of the wild-type NiV-G protein (SEQ ID NO:4), up to 37 contiguous amino acid residues at or near the N-terminus of the wild-type NiV-G protein (SEQ ID NO:4), up to 38 contiguous amino acid residues at or near the N-terminus of the wild-type NiV-G protein (SEQ ID NO: 4), up to 39 contiguous amino acid residues at or near the N-terminus of the wild-type NiV-G protein (SEQ ID NO:4), or up to 40 contiguous amino acid residues at or near the N-terminus of the wild-type NiV-G protein (SEQ ID NO: 4).

In some embodiments, the mutant NiV-G protein has the amino acid sequence set forth in SEQ ID NO: 17 or 18 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 17 or 18. In particular embodiments, the G protein has the sequence of amino acids set forth in SEQ ID NO: 17 or 18. In some embodiments, the mutant NiV-G protein has the amino acid sequence set forth in SEQ ID NO: 17 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 17. In particular embodiments, the G protein has the sequence of amino acids set forth in SEQ ID NO 17. In some embodiments, the mutant NiV-G protein has the amino acid sequence set forth in SEQ ID NO: 18 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 18. In particular embodiments, the G protein has the sequence of amino acids set forth in SEQ ID NO 18.

In some embodiments, the G protein is a mutant G protein containing one or more amino acid substitutions selected from the group consisting of E501A. W504A, Q530A and E533A with reference to numbering set forth in SEQ ID NO:4. In some embodiments, the G protein is a mutant G protein that contains one or more amino acid substitutions elected from the group consisting of E501A. W504A, Q530A and E533A with reference to SEQ ID NO:4 and is a biologically active portion thereof containing an N-terminal truncation.

c. F Proteins

In some embodiments, the targeting moiety comprises a protein with a hydrophobic fusion peptide domain. In some embodiments, the targeting moiety comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein, a bat Paramyxovirus F protein, or a Langya Henipavirus F protein, or a biologically active portion thereof.

Table 2 provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F₀ precursors containing a signal peptide (e.g. corresponding to amino acid residues 1-26 of SEQ ID NO:28). Following cleavage of the signal peptide, the mature F₀ (e.g. SEQ ID NO:29) is transported to the cell surface, then endocytosed and cleaved by cathepsin L into the mature fusogenic subunits F1 and F2. In some embodiments, the signal peptide comprises the amino acid sequence set forth in SEQ ID NO: 38. In some embodiments, the F₀ comprises the amino acid sequence of SEQ ID NO:41. In some embodiments, the F1 subunit comprises the sequence amino acid sequence set forth in SEQ ID NO:46. In some embodiments, the F2 subunit comprises the sequence amino acid sequence set forth in SEQ ID NO:39. The F1 and F2 subunits are associated by a disulfide bond and recycled back to the cell surface. The F1 subunit contains the fusion peptide domain located at the N terminus of the F1 subunit, where it is able to insert into a cell membrane to drive fusion. In some aspects, fusion is blocked by association of the F protein with G protein, until the G protein engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93 (13): e00577-19). In some aspects or the provided re-targeted lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. Journal of Virology. 2019. 93 (13): e00577-19). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 2
F proteins

| | | | SEQ ID |
|---|---|---|---|
| | | | (without |
| Full Gene | | | signal |
| Name | Sequence | SEQ ID | sequence) |
| Hendra virus | MATQEVRLKCLLCGIIVLVLSLEGLGILHYEKLSKIGLV | 28 | 29 |
| F Protein | KGITRKYKIKSNPLTKDIVIKMIPNVSNVSKCTGTVME | | |
| | NYKSRLTGILSPIKGAIELYNNNTHDLVGDVKLAGVV | | |
| | MAGIAIGIATAAQITAGVALYEAMKNADNINKLKSSIE | | |
| | STNEAVVKLQETAEKTVYVLTALQDYINTNLVPTIDQI | | |
| | SCKQTELALDLALSKYLSDLLFVFGPNLQDPVSNSMTI | | |
| | QAISQAFGGNYETLLRTLGYATEDFDDLLESDSIAGQIV | | |
| | YVDLSSYYIIVRVYFPILTEIQQAYVQELLPVSFNNDNS | | |
| | EWISIVPNFVLIRNTLISNIEVKYCLITKKSVICNQDYAT | | |
| | PMTASVRECLTGSTDKCPRELVVSSHVPRFALSGGVLF | | |
| | ANCISVTCQCQTTGRAISQSGEQTLLMIDNTTCTTVVL | | |
| | GNIIISLGKYLGSINYNSESIAVGPPVYTDKVDISSQISS | | |
| | MNQSLQQSKDYIKEAQKILDTVNPSLISMLSMIILYVLS | | |
| | IAALCIGLITFISFVIVEKKRGNYSRLDDRQVRPVSNGD | | |
| | LYYIGT | | |
| Nipah virus | MVVILDKRCYCNLLILILMISECSVGILHYEKLSKIGLV | 30 | 31 |
| F Protein | KGVTRKYKIKSNPLTKDIVIKMIPNVSNMSQCTGSVME | | |
| | NYKTRLNGILTPIKGALEIYKNNTHDLVGDVRLAGVIM | | |
| | AGVAIGIATAAQITAGVALYEAMKNADNINKLKSSIES | | |
| | TNEAVVKLQETAEKTVYVLTALQDYINTNLVPTIDKIS | | |
| | CKQTELSLDLALSKYLSDLLFVFGPNLQDPVSNSMTIQ | | |
| | AISQAFGGNYETLLRTLGYATEDFDDLLESDSITGQIIY | | |
| | VDLSSYYIIVRVYFPILTEIQQAYIQELLPVSFNNDNSEW | | |
| | ISIVPNFILVRNTLISNIEIGFCLITKRSVICNQDYATPMT | | |
| | NNMRECLTGSTEKCPRELVVSSHVPRFALSNGVLFAN | | |
| | CISVTCQCQTTGRAISQSGEQTLLMIDNTTCPTAVLGN | | |
| | VIISLGKYLGSVNYNSEGIAIGPPVFTDKVDISSQISSMN | | |
| | QSLQQSKDYIKEAQRLLDTVNPSLISMLSMIILYVLSIAS | | |
| | LCIGLITFISFIIVEKKRNTYSRLEDRRVRPTSSGDLYYIGT | | |
| Cedar Virus | MSNKRTTVLIIISYTLFYLNNAAIVGFDFDKLNKIGVVQ | 32 | 33 |
| F Protein | GRVLNYKIKGDPMTKDLVLKFIPNIVNITECVREPLSRY | | |
| | NETVRRLLLPIHNMLGLYLNNTNAKMTGLMIAGVIMG | | |
| | GIAIGIATAAQITAGFALYEAKKNTENIQKLTDSIMKTQ | | |
| | DSIDKLTDSVGTSILILNKLQTYINNQLVPNLELLSCRQ | | |
| | NKIEFDLMLTKYLVDLMTVIGPNINNPVNKDMTIQSLS | | |
| | LLFDGNYDIMMSELGYTPQDFLDLIESKSITGQIIYVDM | | |
| | ENLYVVIRTYLPTLIEVPDAQIYEFNKITMSSNGGEYLS | | |
| | TIPNFILIRGNYMSNIDVATCYMTKASVICNQDYSLPMS | | |
| | QNLRSCYQGETEYCPVEAVIASHSPRFALTNGVIFANCI | | |
| | NTICRCQDNGKTITQNINQFVSMIDNSTCNDVMVDKFT | | |
| | IKVGKYMGRKDINNINIQIGPQIIIDKVDLSNEINKMNQ | | |
| | SLKDSIFYLREAKRILDSVNISLISPSVQLFLIIISVLSFIIL | | |
| | LIIIVYLYCKSKHSYKYNKFIDDPDYYNDYKRERINGK | | |
| | ASKSNNIYYVGD | | |
| Mojiang | MALNKNMFSSLFLGYLLVYATTVQSSIHYDSLSKVGVI | 34 | 35 |
| virus, | KGLTYNYKIKGSPSTKLMVVKLIPNIDSVKNCTQKQYD | | |
| Tongguan 1 | EYKNLVRKALEPVKMAIDTMLNNVKSGNNKYRFAGA | | |
| F Protein | IMAGVALGVATAATVTAGIALHRSNENAQAIANMKSA | | |
| | IQNTNEAVKQLQLANKQTLAVIDTIRGEINNNIIPVINQ | | |
| | LSCDTIGLSVGIRLTQYYSEIITAFGPALQNPVNTRITIQ | | |
| | AISSVFNGNFDELLKIMGYTSGDLYEILHSELIRGNIIDV | | |
| | DVDAGYIALEIEFPNLTLVPNAVVQELMPISYNIDGDE | | |
| | WVTLVPRFVLTRTTLLSNIDTSRCTITDSSVICDNDYAL | | |
| | PMSHELIGCLQGDTSKCAREKVVSSYVPKFALSDGLV | | |
| | YANCLNTICRCMDTDTPISQSLGATVSLLDNKRCSVYQ | | |
| | VGDVLISVGSYLGDGEYNADNVELGPPIVIDKIDIGNQL | | |
| | AGINQTLQEAEDYIEKSEEFLKGVNPSIITLGSMVVLYIF | | |
| | MILIAIVSVIALVLSIKLTVKGNVVRQQFTYTQHVPSME | | |
| | NINYVSH | | |
| Bat | MKKKTDNPTISKRGHNHSRGIKSRALLRETDNYSNGLI | 36 | 37 |
| Paramyxovirus | VENLVRNCHHPSKNNLNYTKTQKRDSTIPYRVEERKG | | |
| Eid_hel/GH- | HYPKIKHLIDKSYKHIKRGKRRNGHNGNIITIILLLILILK | | |
| M74a/GHA/ | TQMSEGAIHYETLSKIGLIKGITREYKVKGTPSSKDIVIK | | |
| 2009 F | LIPNVTGLNKCTNISMENYKEQLDKILIPINNIIELYANS | | |
| protein | TKSAPGNARFAGVIIAGVALGVAAAAQITAGIALHEAR | | |
| | QNAERINLLKDSISATNNAVAELQEATGGIVNVITGMQ | | |
| | DYINTNLVPQIDKLQCSQIKTALDISLSQYYSEILTVFGP | | |
| | NLQNPVTTSMSIQAISQSFGGNIDLLLNLLGYTANDLL | | |
| | DLLESKSITGQITYINLEHYFMVIRVYYPIMTTISNAYV | | |
| | QELIKISFNVDGSEWVSLVPSYILIRNSYLSNIDISECLIT | | |
| | KNSVICRHDFAMPMSYTLKECLTGDTEKCPREAVVTS | | |
| | YVPRFAISGGVIYANCLSTTCQCYQTGKVIAQDGSQTL | | |
| | MMIDNQTCSIVRIEEILISTGKYLGSQEYNTMHVSVGNP | | |
| | VFTDKLDITSQISNINQSIEQSKFYLDKSKAILDKINLNLI | | |
| | GSVPISILFIIAILSLILSIITFVIVMIIVRRYNKYTPLINSDP | | |
| | SSRRSTIQDVYIIPNPGEHSIRSAARSIDRDRD | | |

In some embodiments, the F protein is encoded by a nucleotide sequence that encodes the sequence set forth by any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NO:28, SEQ ID NO:29, SEQ ID NO:30, SEQ ID NO:31, SEQ ID NO: 32, SEQ ID NO:33, SEQ ID NO:34, SEQ ID NO:35, SEQ ID NO:36, or SEQ ID NO:37. In some embodiments, the F protein is encoded by a nucleotide sequence that encodes the sequence set forth by any one of SEQ ID NO:28, SEQ ID NO:29, SEQ ID NO:30, SEQ ID NO:31, SEQ ID NO:32, SEQ ID NO: 33, SEQ ID NO:34, SEQ ID NO:35, SEQ ID NO:36, or SEQ ID NO:37.

In particular embodiments, the F protein or the functionally active variant or biologically active portion thereof retains fusogenic activity in conjunction with a Henipavirus G protein, such as a G protein set forth in Section IV.A.2 (e.g. NiV-G or HeV-G). Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO: 30).

In particular embodiments, the F protein has the sequence of amino acids set forth in SEQ ID NO:28, SEQ ID NO:29, SEQ ID NO:30, SEQ ID NO:31, SEQ ID NO:32, SEQ ID NO:33, SEQ ID NO: 34, SEQ ID NO:35, SEQ ID NO:36, or SEQ ID NO:37, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO: 36, or SEQ ID NO:37, and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37.

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NO:28, SEQ ID NO: 29, SEQ ID NO:30, SEQ ID NO:31, SEQ ID NO:32, SEQ ID NO:33, SEQ ID NO:34, SEQ ID NO: 35, SEQ ID NO:36, or SEQ ID NO:37, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:28, SEQ ID NO:29, SEQ ID NO:30, SEQ ID NO: 31, SEQ ID NO:32, SEQ ID NO:33, SEQ ID NO:34, SEQ ID NO:35, SEQ ID NO:36, or SEQ ID NO: 37.

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 28-37. In some embodiments, the mutant F protein is truncated and lacks up to 20 contiguous amino acids, such as up to 19, 18, 17, 16, 15, 14, 13, 12, 11, 10, 9, 8, 7, 6, 5, 4, 3, 2, or 1 contiguous amino acids at the C-terminus of the wild-type F protein. In some embodiments, the mutant F protein comprises the sequence set forth in SEQ ID NO:15. In some embodiments, the mutant F protein comprises the sequence set forth in SEQ ID NO:20. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F₀ precursor is encoded by a sequence of nucleotides encoding the sequence set forth in SEQ ID NO: 20. The encoding nucleic acid can encode a signal peptide sequence that has the sequence MVVILDKRCY CNLLILILMI SECSVG (SEQ ID NO:38). In some examples, the F protein is cleaved into an F1 subunit comprising the sequence set forth in SEQ ID NO:46 and an F2 subunit comprising the sequence set forth in SEQ ID NO:39.

In some embodiments, the F protein is a NiV-F protein that is encoded by a sequence of nucleotides encoding the sequence set forth in SEQ ID NO:30, or is a functionally active variant or biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:30. In some embodiments, the F protein is a NiV-F protein that is encoded by a sequence of nucleotides encoding the sequence set forth in SEQ ID NO:30. In some embodiments, the NiV-F-protein has the sequence of set forth in 30, or is a functionally active variant or a biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to 30. In some embodiments, the NiV-F-protein has the sequence of set forth in 30. In In particular embodiments, the F protein or the functionally active variant or biologically active portion thereof retains the cleavage site cleaved by cathepsin L.

In some embodiments, the F protein or the functionally active variant or the biologically active portion thereof includes an F1 subunit that has the sequence set forth in SEQ ID NO:46, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein or the functionally active variant or biologically active portion thereof includes an F2 subunit that has the sequence set forth in SEQ ID NO:39, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

In some embodiments, the F protein or the functionally active variant or the biologically active portion thereof includes an F1 subunit that has the sequence set forth in SEQ ID NO:46, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein or the functionally active variant or biologically active portion thereof includes an F2 subunit that has the sequence set forth in SEQ ID NO:39, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

In some embodiments, the F protein is a mutant NiV-F protein that is a biologically active portion thereof that is truncated and lacks up to 20 contiguous amino acid residues at or near the C-terminus of the wild-type NiV-F protein (e.g. set forth SEQ ID NO:40). In some embodiments, the mutant NiV-F protein comprises an amino acid sequence set forth in SEQ ID NO:20. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:20. In some embodiments, the mutant F protein contains an F1 protein that has the sequence set forth in SEQ ID NO:46. In some embodiments, the mutant F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein is a mutant NiV-F protein that is a biologically active portion thereof that comprises a 20 amino acid truncation at or near the C-terminus of the wild-type NiV-F protein (SEQ ID NO:40); and a point mutation on an N-linked glycosylation site. In some embodiments, the mutant NiV-F protein comprises an amino acid sequence set forth in SEQ ID NO:15. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 15.

In some embodiments, the F protein is a mutant NiV-F protein that is a biologically active portion thereof that comprises a 25 amino acid truncation at or near the C-terminus of the wild-type NiV-F protein (SEQ ID NO:40). In some embodiments, the F protein is a mutant NiV-F protein that is a biologically active portion thereof that comprises a 22 amino acid truncation at or near the C-terminus of the wild-type NiV-F protein (SEQ ID NO:40). In some embodiments, the NiV-F protein is encoded by a nucleotide sequence that encodes the sequence set forth in SEQ ID NO:20. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:20.

In some embodiments, the F protein is a mutant NiV-F protein that is a biologically active portion thereof that comprises a 22 amino acid truncation at or near the C-terminus of the wild-type NiV-F protein (SEQ ID NO:40). In some embodiments, the NiV-F protein comprises the amino acid sequence set forth in SEQ ID NO:21, or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:21. In some embodiments, the NiV-F protein is encoded by a nucleotide sequence that encodes the sequence set forth in SEQ ID NO:21. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:21.

### Targeting Moieties

In some embodiments, a particle described herein comprises a targeting moiety (i.e., a binding agent).

In some embodiments, the targeting moiety can be any agent that binds to a cell surface molecule on a target cells. In some embodiments, the targeting moiety can be an antibody or an antibody portion or fragment.

The targeting moiety may be modulated to have different binding strengths. For example, scFvs and antibodies with various binding strengths may be used to alter the fusion activity of the chimeric attachment proteins towards cells that display high or low amounts of the target antigen. For example DARPins with different affinities may be used to alter the fusion activity towards cells that display high or low amounts of the target antigen. Targeting moietys may also be modulated to target different regions on the target ligand, which will affect the fusion rate with cells displaying the target.

The targeting moiety may comprise a humanized antibody molecule, intact IgA, IgG, IgE or IgM antibody; bi- or multi-specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

In some embodiments, the targeting moiety is a single chain molecule. In some embodiments, the targeting moiety is a single domain antibody. In some embodiments, the targeting moiety is a single chain variable fragment. In particular embodiments, the targeting moiety contains an antibody variable sequence(s) that is human or humanized.

In some embodiments, the targeting moiety is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from phage display libraries. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the single domain antibody is attached to the C-terminus of the G protein or biologically active portion thereof. In some embodiments, the N-terminus of the single domain antibody is exposed on the exterior surface of the lipid bilayer. In some embodiments, the N-terminus of the single domain antibody binds to a cell surface molecule of a target cell. In some embodiments, the single domain antibody specifically binds to a cell surface molecule present on a target cell. In some embodiments, the cell surface molecule is a protein, glycan, lipid or low molecular weight molecule.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells. T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes,

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cadiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the cell surface molecule is any one of CD3, CD8, CD4, asialoglycoprotein receptor 2 (ASGR2), transmembrane 4 L6 family member 5 (TM4SF5), low density lipoprotein receptor (LDLR) or asialoglycoprotein 1 (ASGR1).

In some embodiments, a particle is re-targeted by virtue of the binding agent (e.g., a CD3-, CD8-, or CD4-binding agent). For example, in some cases, a lipid particle comprises a fusogen to facilitate the fusion of the particle to the membrane, and the fusogen is modified to comprise the binding agent to re-target the particle to a target cell (e.g., a CD3-, CD8-, or CD4-expressing cell).

### A. CD3 Binding Agents

In some embodiments, the particles disclosed herein include one or more CD3 binding agents. For example, a CD3 binding agent may be fused to or incorporated into a protein fusogen or viral envelope protein. In another embodiment, a CD3 binding agent may be incorporated into the viral envelope via fusion with a transmembrane domain.

Exemplary CD3 binding agents include antibodies and fragments thereof (e.g., scFv, VHH) that bind to CD3. Such antibodies may be derived from any species, and may be for example, mouse, rabbit, human, humanized, or camelid antibodies.

Exemplary antibodies include OKT3, CRIS-7, 12C, blinatumomab, catumaxomab, muromonab-CD3, A-319, AFM11, AMG 199, AMG 211, AMG 424, AMG 427, AMG 562, AMG 564, APVO436, CC-93269, ERY974, GBR1302, GEM333, GEM2PSCA, GNC-035, HPN424, IGM-2323, JNJ-63709178, JNJ-63898081, JNJ-75348780, JNJ-78306358, M701, M802, MGD007, MOR209/ES414, PF-06671008, REGN5459, RO7283420, SAR442257, SAR443216, TNB-383B, TNB-486, TNB-585, Y150, acapatamab, cevostamab, cibisatamab, duvortuxizumab, eluvixtamab, emerfetamab, etevritamab, glofitamab, gresonitamab, obrindatamab, pavurutamab, plamotamab, solitomab, tarlatamab, tepoditamab, tidutamab, vibecotamab, vixtimotamab, alnuctamab, dafsolimab setaritox, pacanalotamab, pasotuxizumab, runimotamab, nivatrotamab, elranatamab, ertumaxomab, flotetuzumab, odronextamab, talquetamab, teclistamab, visilizumab, epcoritamab, otelixizumab, 3F8BiAb, CCW702, DKTK CC-1, EMB-06, GEN1044, GEN1047, GTB-3550, HPN217, IMC-C103C, NVG-111, REGN4018, REGN4336, REGN5458, A-2019, A-337, ABP-100, AFM15, AFM21, AMG 701. APVO425, CLN-049, Dow2, EM801, Ektomab, FBTA05, GBR1342, GBR1372, GSK3537142, HBM7020, HLX31, IGM-2644, MG1122, MGD015, ND003, ND007, PF-07062119, RO7293583, STA551. TT19, ZW38; and anti-CD3 antibodies disclosed in U.S. Pat. Nos. 4,361,549, 7,728,114, 9,657,102, 9,587,021, and 11,007,267; US Patent Application Nos. US20120269826, US20180057597, and US20180112000; and PCT Application Nos. WO2005118635, WO2011050106, WO2012162067. WO2014047231. WO2016116626, WO2016180721, and WO2016204966. Other exemplary binding agents include designed ankyrin repeat proteins (DARPins) and binding agents based on fibronectin type III (Fn3) scaffolds.

In some embodiments, the CD3 binding agent comprises a heavy chain variable (VH) region comprising a CDR-H1, a CDRH-2, and a CDR-H3 comprising the amino acid sequence set forth in SEQ ID NO: 118, 119, and 120, respectively; and a light chain variable region comprising a CDR-L1, a CDR-L2, and a CDR-L3 comprising the amino acid sequence set forth in SEQ ID NO: 121, 122, and 123, respectively. In some embodiments, the CD3 binding agent comprises a VH region comprising an amino acid sequence having at least about 90% sequence identity to the amino acid sequence set forth in SEQ ID NO: 124, and a VL region comprising an amino acid sequence having at least about 90% sequence identity to the amino acid sequence set forth in SEQ ID NO: 125. In some embodiments, the CD3 binding agent comprises a VH region comprising the amino acid sequence set forth in SEQ ID NO: 124, and a VL region comprising the amino acid sequence set forth in SEQ ID NO: 125. In some embodiments, the CD3 binding agent is an scFv. In some embodiments, the CD3 binding agent comprises the amino acid sequence set forth in SEQ ID NO: 126. In some embodiments, the CD3 binding agent is OKT3.

In some embodiments, the CD3 binding agent is activating (e.g., the CD3 binding agent activates T cells). In some embodiments, the CD3 binding agent is non-activating (e.g., it does not activate T cells).

In some embodiments, protein fusogens or viral envelope proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the lipid particle. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt 1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD3 binding agent to the fusion protein or targeting protein (e.g. the hemagglutinin protein). In some embodiments, the fusogen and CD3 binding agent are covalently conjugated by expression of a chimeric protein comprising the fusogen linked to the CD3 binding agent. In some embodiments, a single-chain variable fragment (scFv) can be conjugated to fusogens to redirect fusion activity towards cells that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, a single domain antibody (e.g., a VHH) can be conjugated to fusogens to redirect fusion activity towards cells that display the sdAb binding target. In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD3 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector, VLP, or gesicle (doi: 10.1016/j.biomaterials.2014.01.051).

In some embodiments, a CD3 binding agent comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′); fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPsTM”); single chain or

Tandem diabodies (TandAb®); VHHs; Anticalins®; Nanobodies®; minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®S.

In some embodiments, the CD3 binding agent is a peptide. In some embodiments, the CD3 binding agent is an antibody, such as a single-chain variable fragment (scFv). In some embodiments, the CD3 binding agent is an antibody, such as a single domain antibody. In some embodiments, the antibody can be human or humanized. In some embodiments, the CD3 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD3 binding agent is attached to the C-terminus of the G protein (e.g., fusogen) or biologically active portion thereof. In some embodiments, the N-terminus of the CD3 binding agent is exposed on the exterior surface of the lipid bilayer.

In some embodiments, the CD3 binding agent is the only surface displayed non-viral sequence of the lipid particle. In some embodiments, the CD3 binding agent is the only membrane bound non-viral sequence of the lipid particle. In some embodiments, the lipid particle does not contain a molecule that engages or stimulates T cells other than the CD3 binding agent. In some embodiments, the lipid particle contains a non-activating CD3 binding agent.

In some embodiments, lipid particles may display CD3 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

### B. CD8 Binding Agents

In some embodiments, the particles disclosed herein include one or more CD8 binding agents. For example, a CD8 binding agent may be fused to or incorporated into a protein fusogen or viral envelope protein. In another embodiment, a CD8 binding agent may be incorporated into the viral envelope via fusion with a transmembrane domain.

Exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2019032661, WO2020257412, WO2018170096, WO2020060924, U.S. Pat. No. 10,730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SK1, RPA-T8 (GeneTex); UCHT4 (Absolute Antibody); BW135/80 (Miltenyi); G42-8 (BD Biosciences); C8/1779R, mAB 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Other exemplary binding agents include designed ankyrin repeat proteins (DARPins) and binding agents based on fibronectin type III (Fn3) scaffolds.

In some embodiments, the CD8 binding agent comprises a CDR-H1, a CDR-H2, and a CDR-H3 comprising the amino acid sequence set forth in SEQ ID NO: 85, 86, and 87, respectively; and a CDR-L1, a CDR-L2, and a CDR-L3 comprising the amino acid sequence set forth in SEQ ID NO: 88, 89, and 90, respectively. In some embodiments, the CD8 binding agent comprises a heavy chain variable region (VH) comprising the amino acid sequence set forth in SEQ ID NO:91, and a light chain variable region (VL) comprising the amino acid sequence set forth in SEQ ID NO:92. In some embodiments, the CD8 binding agent comprises the sequence set forth in SEQ ID NO:80.

In some embodiments, the CD8 binding agent comprises a CDR-H1, a CDR-H2, and a CDR-H3 comprising the amino acid sequence set forth in SEQ ID NO: 93, 94, and 95, respectively; and a CDR-L1, a CDR-L2, and a CDR-L3 comprising the amino acid sequence set forth in SEQ ID NO: 96, 97, and 98, respectively. In some embodiments, the CD8 binding agent comprises a heavy chain variable region (VH) comprising the amino acid sequence set forth in SEQ ID NO:99, and a light chain variable region (VL) comprising the amino acid sequence set forth in SEQ ID NO: 100. In some embodiments, the CD8 binding agent comprises the sequence set forth in SEQ ID NO:81.

In some embodiments, the CD8 binding agent comprises a CDR-H1, a CDR-H2, and a CDR-H3 comprising the amino acid sequence set forth in SEQ ID NO: 101, 102, and 103, respectively; and a CDR-L1, a CDR-L2, and a CDR-L3 comprising the amino acid sequence set forth in SEQ ID NO: 88, 89, and 104, respectively. In some embodiments, the CD8 binding agent comprises a heavy chain variable region (VH) comprising the amino acid sequence set forth in SEQ ID NO: 105, and a light chain variable region (VL) comprising the amino acid sequence set forth in SEQ ID NO: 106. In some embodiments, the CD8 binding agent comprises the sequence set forth in SEQ ID NO:82.

In some embodiments, the CD8 binding agent comprises a CDR-H1, a CDR-H2, and a CDR-H3 comprising the amino acid sequence set forth in SEQ ID NO: 107, 108, and 109, respectively; and a CDR-L1, a CDR-L2, and a CDR-L3 comprising the amino acid sequence set forth in SEQ ID NO: 110, 111, and 112, respectively. In some embodiments, the CD8 binding agent comprises a heavy chain variable region (VH) comprising the amino acid sequence set forth in SEQ ID NO:113, and a light chain variable region (VL) comprising the amino acid sequence set forth in SEQ ID NO:114. In some embodiments, the CD8 binding agent comprises the sequence set forth in SEQ ID NO:83.

In some embodiments, the CD8 binding agent comprises a CDR-H1, a CDR-H2, and a CDR-H3 comprising the amino acid sequence set forth in SEQ ID NO: 115, 116, and 117, respectively. In some embodiments, the CD8 binding agent comprises a heavy chain variable region (VH) comprising the amino acid sequence set forth in SEQ ID NO:117. In some embodiments, the CD8 binding agent comprises the sequence set forth in SEQ ID NO:117. In some embodiments, the CD8 binding agent comprises the sequence set forth in any one of SEQ ID NOS: 80, 81, 82, 83, or 84. In some embodiments, the CD8 binding agent comprises the sequence set forth in SEQ ID NO:80. In some embodiments, the CD8 binding agent comprises the sequence set forth in SEQ ID NO:81. In some embodiments, the CD8 binding agent comprises the sequence set forth in SEQ ID NO:82. In some embodiments, the CD8 binding agent comprises the sequence set forth in SEQ ID NO:83. In some embodiments, the CD8 binding agent comprises the sequence set forth in SEQ ID NO:84.

In some embodiments, the CD8 binding agent comprises any CD8 binding agent as described in US 2019/0144885, incorporated by reference herein in its entirety.

In some embodiments, protein fusogens or viral envelope proteins may be re-targeted by mutating amino acid residues in a fusion protein or a targeting protein (e.g. the hemagglutinin protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the lipid particle. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD8 binding agent to the fusion protein or targeting protein (e.g. the hemagglutinin protein). In some embodiments, the fusogen and CD8 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi: 10.1038/nbt1060, DOI 10.1182/blood-2012-11-468579, doi: 10.1038/nmeth. 1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD8 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector, VLP, or gesicle (doi: 10.1016/j.biomaterials.2014.01.051).

In some embodiments, a CD8 binding agent comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′); fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BITE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®S.

In some embodiments, the CD8 binding agent is a peptide. In some embodiments, the CD8 binding agent is an antibody, such as a single-chain variable fragment (scFv). In some embodiments, the CD8 binding agent is an antibody, such as a single domain antibody. In some embodiments, the CD8 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD8 binding agent is attached to the C-terminus of the G protein (e.g., fusogen) or biologically active portion thereof. In some embodiments, the N-terminus of the CD8 binding agent is exposed on the exterior surface of the lipid bilayer.

In some embodiments, the CD8 binding agent is the only surface displayed non-viral sequence of the lipid particle. In some embodiments, the CD8 binding agent is the only membrane bound non-viral sequence of the lipid particle. In some embodiments, the lipid particle does not contain a molecule that engages or stimulates T cells other than the CD8 binding agent.

In some embodiments, lipid particles may display CD8 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

### C. CD4 Binding Agents

In some embodiments, the particles disclosed herein include one or more CD4 binding agents. For example, a CD4 binding agent may be fused to or incorporated in a protein fusogen or viral envelope protein. In another embodiment, a CD4 binding agent may be incorporated into the viral envelope via fusion with a transmembrane domain.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621. U.S. Pat. Nos. 8,911,728, 9,587,022, 9,745,552; as well as antibodies B486A1, RPA-T4, CE9.1 (Novus Biologicals); GK1.5, RM4-5, RPA-T4, OKT4, 4SM95, S3.5, N1UG0 (ThermoFisher); GTX50984, ST0488, 10B5, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds.

In some embodiments, protein fusogens or viral envelope proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the lipid particle. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD4 binding agent to the fusion protein or targeting protein (e.g. the hemagglutinin protein). In some embodiments, the fusogen and CD4 binding agent are covalently conjugated by expression of a chimeric protein comprising the fusogen linked to the CD4 binding agent. In some embodiments, a single-chain variable fragment (scFv) can be conjugated to fusogens to redirect fusion activity towards cells that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD4 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector, VLP, or gesicle (doi: 10.1016/j.biomaterials.2014.01.051).

In some embodiments, a CD4 binding agent comprises a humanized antibody molecule, intact IgA. IgG, IgE or IgM antibody; bi- or multi-specific antibody (e.g., Zybodies®, etc); antibody fragments such as Fab fragments, Fab′ fragments, F(ab′); fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD4 binding agent is a peptide. In some embodiments, the CD4 binding agent is an antibody, such as a single-chain variable fragment (scFv). In some embodiments, the CD4 binding agent is an antibody, such as a single domain antibody. In some embodiments, the antibody can be human or humanized. In some embodiments, the CD4 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD4 binding agent is attached to the C-terminus of the G protein (e.g., fusogen) or biologically active portion thereof. In some embodiments, the N-terminus of the CD4 binding agent is exposed on the exterior surface of the lipid bilayer.

In some embodiments, the CD4 binding agent is the only surface displayed non-viral sequence of the lipid particle. In some embodiments, the CD4 binding agent is the only membrane bound non-viral sequence of the lipid particle. In some embodiments, the lipid particle does not contain a molecule that engages or stimulates T cells other than the CD4 binding agent.

In some embodiments, lipid particles may display CD4 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

### Pharmaceutical Compositions and Methods of Manufacture

Also provided are compositions containing the lipid particles herein, including pharmaceutical compositions and formulations. The pharmaceutical compositions can include any of the described lipid particles.

The present disclosure also provides, in some aspects, a pharmaceutical composition comprising the composition described herein and pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular lipid particle and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the lipid particle meets a pharmaceutical or good manufacturing practices (GMP) standard. In some embodiments, the lipid particle is made according to good manufacturing practices (GMP). In some embodiments, the lipid particle has a pathogen level below a predetermined reference value, e.g., is substantially free of pathogens. In some embodiments, the lipid particle has a contaminant level below a predetermined reference value, e.g., is substantially free of contaminants. In some embodiments, the lipid particle has low immunogenicity.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, the lipid particle is a viral vector or virus-like particle (e.g., Section II.B.1). In some embodiments, the compositions provided herein can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25 (2): 115-25, 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10¹⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive In some embodiments, the dosage of administration of a viral vector or virus-like particle is from about 10⁵ to about 10⁹ infectious units. In some embodiments, the dosage of administration of a viral vector or virus-like particle is from about 10⁶ to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID₅₀) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive In some embodiments, the dosage of administration of a viral vector or virus-like particle is from about 10⁵ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired response.

The exact amount of vehicle provided lipid particle required will vary from subject to subject, depending on the species, age, weight and general condition of the subject, the particular polynucleic acid, polypeptide, or vector used, its mode of administration etc. TAn appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the lipid particles in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, vehicle formulations may comprise cryoprotectants. As used herein, there term “cryoprotectant” refers to one or more agent that when combined with a given substance, helps to reduce or eliminate damage to that substance that occurs upon freezing. In some embodiments, cryoprotectants are combined with vector vehicles in order to stabilize them during freezing. In some aspects, frozen storage of RNA between −20° C. and −80° C. may be advantageous for long term (e.g. 36 months) stability of polynucleotide. In some embodiments, the RNA species is mRNA. In some embodiments, cryoprotectants are included in vehicle formulations to stabilize polynucleotide through freeze/thaw cycles and under frozen storage conditions. Cryoprotectants of the provided embodiments may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

### Methods of Use and Therapeutic Applications

In some embodiments, the lipid particles (e.g. lentiviral particles, VLPs, or gesicles) provided herein are used for delivery of a heterologous agent (e.g., a heterologous protein, a heterologous nucleic acid per se, or a nucleic acid sequence encoding a heterologous protein) to a target cell. The heterologous agent can be a protein, nucleic acid, such as DNA or RNA (e.g., mRNA), or small molecule. Exemplary heterologous agents that can be contained in a non-cell particle herein for delivery are described. Among provided methods herein are methods that comprise delivering a heterologous agent (e.g. a heterologous protein, a heterologous nucleic acid per se, or a nucleic acid encoding the same) to a target cell. In some embodiments, the heterologous agent is an agent that is entirely heterologous or not produced or normally expressed by the target cell.

In some embodiments, delivery is by transduction of a lipid particle into the target cell. Hence, also provided herein are methods of transduction of a target cell with a provided lipid particles, including those pseudotyped with a viral envelope glycoprotein (e.g., VSV-G) comprising contacting the target cell with the lipid particle as defined above under conditions to effect the transduction of the target cell by the lipid particle. In some embodiments, transduction with a lipid particle (e.g. lentiviral vector particle, VLP, or gesicle) initially delivers the biological material to the membrane or the cytoplasm of the target cell, upon being bound to the target cell. After delivery, the biological material can be translocated to another compartment of the cell. In some embodiments, transduction mediates integration of an exogenous gene expressed by the particle into the genome of the cell. Conditions to effect the transduction of the targeted cells are well-known from the skilled person and include typically incubating the cells to be transduced, such as by culture in flasks, plates or dishes an in some cases in the presence of a transduction adjuvant (e.g. retronectin). In some embodiments, the target cells may be prestimulated or activated, such as with cytokine cocktails or other stimulatory agents for stimulating or activating the target cells. In some embodiments, the lipid particles are incubated with the target cells at an MOI of 1, 5, 10 or 100, or any value between any of the foregoing. In some embodiments, the incubation is in serum-free medium.

Exemplary target cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes,

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cadiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the target cell is a hematopoietic cell. In some embodiments, the hematopoietic cell is a blood cell, such as from the myeloid or the lymphoid lineage. In particular, the hematopoietic cell may be an undifferentiated or poorly differentiated cells such as hematopoietic stem cells and progenitor cells, or differentiated cells such as T lymphocytes, B lymphocytes or dendritic cells. In some embodiments, the hematopoietic cell is selected from the group consisting of hematopoietic stem cells, CD34+ progenitor cells, in particular peripheral blood CD34+ cells, very early progenitor CD34+ cells, B-cell CD19+ progenitors, myeloid progenitor CD13+ cells, T lymphocytes, B lymphocytes, monocytes, dendritic cells, cancer B cells in particular B-cell chronic lymphocytic leukemia (BCLL) cells and marginal zone lymphoma (MZL) B cells, and thymocytes.

In some embodiments, the target cell is a T cell. In some embodiments, the T cell is a resting or quiescent T cell. In some embodiments, the T cell is a naive or a memory T cell. In some embodiments, the T cell has not been activated prior to the delivery of the lipid particles, including prior to transduction with a provided lentiviral vector particle. Thus, in aspects of the provided methods, T cells are not activated with a T cell stimulatory agent such as with an anti-CD3/anti-CD28 antibody reagent (e.g. Dynabeads) prior to their transduction with a provided lipid particle (e.g. lentiviral vector particle). The T cell may be a CD4+ T cell or a CD8+ T cell or a subset thereof.

In some embodiments, the target cell is a B cell. In some embodiments, the B cell is a resting B cell, such as a naïve ro memory B cell. In some embodiments, the B cell may be a cancer B cell, such as a B-cell chronic lymphocytic leukemia (BCLL) cell or a marginal zone lymphoma (MZL) B cell.

In some embodiments, delivery of the heterologous agent (e.g., heterologous protein) to the target cell can provide a therapeutic effect to treat a disease or condition in the subject. The therapeutic effect may be by targeting, modulating or altering an antigen or protein present or expressed by the target cell that is associated with or involved in a disease or condition. The therapeutic effect may be by providing an heterologous agent that is a protein (or a nucleic acid encoding the protein, e.g., an mRNA encoding the protein) which is absent, mutant, or at a lower level than wild-type in the target cell. In some embodiments, the target cell is from a subject having a genetic disease, e.g., a monogenic disease, e.g., a monogenic intracellular protein disease.

In some embodiments, the target cell is from a subject having a hematopoietic disease or disorder. In some embodiments, the hematopoietic disorder may be due to a blood disease, in particular disease involving hematopoietic cells. In some embodiments, the hematopoietic disorder is a monogenic hematopoietic disease, such as due to mutation of a single gene. In some embodiments, the hematopoietic disorder is myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS.

In some embodiments, the target cell is from a subject having an autoimmune disease. In some embodiments, the autoimmune disease is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenia purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, cosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenia purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia. Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis.

In some embodiments, the target cell is from a subject having a cancer. In some embodiments, the cancer is leukemia or a lymphoma.

In some embodiments, the target cell is from a subject having a demyelinating disease of the central nervous system.

The lipid particles, e.g., lentiviral vectors, VLPs, gesicles, or compositions containing the same, described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein). In some embodiments, the disease or condition may be one that is treated by delivery of the heterologous agent contained in the administered lipid particle to a target cell in the subject.

In some embodiments, this disclosure provides, in certain aspects, a method of administering a lipid particle composition to a subject (e.g., a human subject), comprising administering to the subject, a provided lipid particle composition comprising a plurality of lipid particles described herein, thereby administering the lipid particle composition to the subject.

In some embodiments, this disclosure provides, in certain aspects, a method of delivering a lipid particle composition to target cells, comprising contacting a target cell with a provided lipid particle composition comprising a plurality of lipid particles described herein, thereby delivering the lipid particle composition to the target cell. In some embodiments, the contacting is carried out by administering a provided lipid particle to a subject, in which the lipid particle is delivered to the target cell present in the subject.

In some embodiments, the disclosure provides, in certain aspects, a method of delivering a heterologous agent, for instance a heterologous protein or nucleic acid encoding the same, to a subject or a cell, comprising administering to the subject, a plurality of lipid particles described herein, or a pharmaceutical composition described herein, wherein the lipid particle composition is administered in an amount and/or time such that the therapeutic agent is delivered. Exemplary heterologous agents, including heterologous proteins and nucleic acids encoding the same, that can be contained in a lipid particle herein for delivery to a subject are described in Section III.

In some embodiments, the disclosure provides, in certain aspects, a method of delivering a heterologous agent, for instance a heterologous protein or nucleic acid encoding the same, to a target cell, comprising contacting a target cells with a plurality of lipid particles described herein, or a composition described herein, wherein the lipid particle composition is contacted with the target cell under conditions such that the heterologous agent is delivered. Heterologous exogenous agents that can be contained in a lipid particle herein for delivery to a subject are described in Section III. In some embodiments, the contacting is carried out by administering a provided lipid particle to a subject, in which the heterologous agent contained in the lipid particle is delivered to the target cell present in the subject.

In some embodiments, delivery of a heterologous agent by administration of a lipid particle composition described herein may modify cellular protein expression levels. In certain embodiments, the administered composition directs upregulation of (via expression in the cell, delivery in the cell, or induction within the cell) of one or more heterologous agent cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs downregulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the lipid particle composition comprises an exogenous protein), the effect lasts for less than 1, 2, 3, 4, 5, 6, or 7 days, 2, 3, or 4 weeks, or 1, 2, 3, 6, or 12 months.

In some embodiments, the lipid particle further comprises, or the method further comprises delivering, a second heterologous agent that comprises or encodes a second cell surface ligand or antibody that binds a cell surface receptor, and optionally further comprising or encoding one or more additional cell surface ligands or antibodies that bind a cell surface receptor (e.g., 1, 2, 3, 4, 5, 10, 20, 50, or more). In some embodiments, the first heterologous agent and the second heterologous agent form a complex, wherein optionally the complex further comprises one or more additional cell surface ligands. In some embodiments, the heterologous agent comprises or encodes a cell surface receptor, e.g., an exogenous cell surface receptor. In some embodiments, the lipid particle further comprises, or the method further comprises delivering, a second heterologous agent that comprises or encodes a second cell surface receptor, and optionally further comprises or encodes one or more additional cell surface receptors (e.g., 1, 2, 3, 4, 5, 10, 20, 50, or more cell surface receptors).

In some embodiments, the lipid particle is capable of delivering (e.g., delivers) one or more cell surface receptors to a target cell (e.g., an immune cell). Similarly, in some embodiments, a method herein comprises delivering one or more cell surface receptors to a target cell. In some embodiments, the first heterologous agent and the second heterologous agent form a complex, wherein optionally the complex further comprises one or more additional cell surface receptors. In some embodiments, the heterologous agent comprises or encodes an antigen or an antigen presenting protein.

In some embodiments, the lipid particle is capable of causing (e.g., causes) a target cell to secrete a protein, e.g., a therapeutic protein. In some embodiments, the lipid particle is capable of delivering (e.g., delivers) a secreted heterologous agent, e.g., a secreted protein to a target site (e.g., an extracellular region), e.g., by delivering a nucleic acid (e.g., mRNA) encoding the protein to the target cell under conditions that allow the target cell to produce and secrete the protein. Similarly, in some embodiments, a method herein comprises delivering a secreted heterologous agent as described herein. In embodiments, the secreted protein comprises a protein therapeutic, e.g., an antibody molecule, a cytokine, or an enzyme. In embodiments, the secreted protein comprises an autocrine signaling molecule or a paracrine signaling molecule. In embodiments, the secreted heterologous agent comprises a secretory granule.

In some embodiments, the lipid particle is capable of secreting (e.g., secretes) a heterologous agent, e.g., a protein. In some embodiments, the heterologous agent, e.g., secreted agent, is delivered to a target site in a subject. In some embodiments, the heterologous agent is a protein that cannot be made recombinantly or is difficult to make recombinantly. In some embodiments, the lipid particle that secretes a protein is from a source cell selected from an MSC or a chondrocyte.

In some embodiments, the lipid particle is capable of reprogramming (e.g., reprograms) a target cell (e.g., an immune cell), e.g., by delivering an heterologous agent selected from a transcription factor, a nucleic acid encoding a transcription factor, mRNA, or a plurality of said heterologous agents. Similarly, in some embodiments, a method herein comprises reprogramming a target cell. In embodiments, reprogramming comprises inducing an exhausted T cell to take on one or more characteristics of a nonexhausted T cell, e.g., a killer T cell. In some embodiments, the heterologous agent comprises an antigen. In some embodiments, the lipid particle comprises a first heterologous agent comprising an antigen and a second heterologous agent comprising an antigen presenting protein.

In some embodiments, a lipid particle is capable of modifying, e.g., modifies, a target tumor cell, for instance by delivering a heterologous agent (protein or nucleic acid) or a nucleic encoding a heterologous agent. Similarly, in some embodiments, a method herein comprises modifying a target tumor cell. In embodiments, the lipid particle delivers an mRNA encoding an immunostimulatory ligand, an antigen presenting protein, a tumor suppressor protein, or a pro-apoptotic protein. In some embodiments, the lipid particle delivers an miRNA capable of reducing levels in a target cell of an immunosuppressive ligand, a mitogenic signal, or a growth factor.

In some embodiments, a lipid particle delivers a heterologous agent that is immunomodulatory, e.g., immunostimulatory.

In some embodiments, a lipid particle is capable of causing (e.g., causes) the target cell to present an antigen, for instance by delivering a heterologous agent comprising an antigen or a nucleic acid encoding the antigen. Similarly, in some embodiments, a method herein comprises presenting an antigen on a target cell. In some embodiments, the lipid particle promotes regeneration in a target tissue. Similarly, in some embodiments, a method herein comprises promoting regeneration in a target tissue.

In some embodiments, the lipid particle is capable of delivering (e.g., delivers) a nucleic acid to a target cell, e.g., to stably modify the genome of the target cell, e.g., for gene therapy. Similarly, in some embodiments, a method herein comprises delivering a nucleic acid to a target cell. In some embodiments, the target cell has an enzyme deficiency, e.g., comprises a mutation in an enzyme leading to reduced activity (e.g., no activity) of the enzyme.

In some embodiments, the lipid particle is capable of delivering (e.g., delivers) a reagent that mediates a sequence specific modification to DNA (e.g., Cas9, ZFN, or TALEN) in the target cell. Similarly, in some embodiments, a method herein comprises delivering the reagent to the target cell. In embodiments, the target cell is a CNS cell.

In some embodiments, the lipid particle is capable of delivering (e.g., delivers) a nucleic acid to a target cell, e.g., to transiently modify gene expression in the target cell.

In some embodiments, the lipid particle is capable of delivering (e.g., delivers) a protein to a target cell, e.g., to transiently rescue a protein deficiency. Similarly, in some embodiments, a method herein comprises delivering a protein to a target cell. In embodiments, the protein is a membrane protein (e.g., a membrane transporter protein), a cytoplasmic protein (e.g., an enzyme), or a secreted protein (e.g., an immunosuppressive protein).

In some embodiments, the lipid particle is capable of intracellular molecular delivery, e.g., delivers a protein heterologous agent to a target cell. Similarly, in some embodiments, a method herein comprises delivering a molecule to an intracellular region of a target cell. In embodiments, the protein heterologous agent is an inhibitor. In some embodiments, the protein heterologous agent comprises a nanobody, scFv, camelid antibody, peptide, macrocycle, or small molecule.

In some embodiments, the lipid particle comprises on its membrane one or more cell surface ligands (e.g., 1, 2, 3, 4, 5, 10, 20, 50, or more cell surface ligands), said cell surface ligands to be presented by the lipid particle to a target cell. Similarly, in some embodiments, a method herein comprises presenting one or more cell surface ligands to a target cell. In some embodiments, the lipid particle having a cell surface ligand is from a source cell chosen from a neutrophil (e.g., and the target cell is a tumor-infiltrating lymphocyte), dendritic cell (e.g., and the target cell is a naive T cell), or neutrophil (e.g., and the target is a tumor cell or virus-infected cell). In some embodiments the lipid particle comprises a membrane complex, e.g., a complex comprising at least 2, 3, 4, or 5 proteins, e.g., a homodimer, heterodimer, homotrimer, heterotrimer, homotetramer, or heterotetramer. In some embodiments, the lipid particle comprises an antibody, e.g., a toxic antibody, e.g., the lipid particle is capable of delivering the antibody to the target site, e.g., by homing to a target site. In some embodiments, the source cell is an NK cell or neutrophil.

In some embodiments, a method herein comprises causing ligand presentation on the surface of a target cell by presenting cell surface ligands on the lipid particle. In some embodiments, the lipid particle is capable of causing cell death of the target cell. In some embodiments, the lipid particle is from a NK source cell. In some embodiments, a lipid particle or target cell is capable of phagocytosis (e.g., of a pathogen). Similarly, in some embodiments, a method herein comprises causing phagocytosis. In some embodiments, a lipid particle senses and responds to its local environment. In some embodiments, the lipid particle is capable of sensing level of a metabolite, interleukin, or antigen.

In embodiments, a lipid particle is capable of chemotaxis, extravasation, or one or more metabolic activities. In embodiments, the metabolic activity is selected from kyneurinine, gluconeogenesis, prostaglandin fatty acid oxidation, adenosine metabolism, urea cycle, and thermogenic respiration. In some embodiments, the source cell is a neutrophil and the lipid particle is capable of homing to a site of injury. In some embodiments, the source cell is a macrophage and the lipid particle is capable of phagocytosis. In some embodiments, the source cell is a brown adipose tissue cell and the lipid particle is capable of lipolysis.

In some embodiments, the lipid particle comprises (e.g., is capable of delivering to the target cell) a plurality of heterologous agents (e.g., at least 2, 3, 4, 5, 10, 20, or 50 heterologous agents) or nucleic acids encoding a plurality of heterologous agents. In embodiments, the lipid particle comprises an inhibitory nucleic acid (e.g., siRNA or miRNA) and an mRNA.

In some embodiments, the lipid particle comprises (e.g., is capable of delivering to the target cell) a membrane protein or a nucleic acid encoding the membrane protein. In embodiments, the lipid particle is capable of reprogramming or transdifferentiating a target cell, e.g., the lipid particle comprises one or more agents that induce reprogramming or transdifferentiation of a target cell.

### EXEMPLARY EMBODIMENTS

Among the provided embodiments are:
1. A lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a RNA sequence encoding a gag protein or portion thereof comprising at least a gag start codon; a RNA sequence encoding a heterologous protein that is operably linked to the RNA sequence encoding a gag protein or portion thereof; and a poly-A tail,
wherein each of the R element of the 5′ LTR, the U5 element of the 5′ LTR, and the RNA sequence encoding a gag protein or portion thereof is retroviral.
2. A lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a gag 5′ untranslated region (UTR) or portion thereof comprising at least three nucleotides; a RNA sequence encoding a heterologous protein that is operably linked to the gag 5′ UTR or a portion thereof; and a poly-A tail,
wherein each of the R element of the 5′ LTR, the U5 element of the 5′ LTR, and the gag 5′ UTR or portion thereof is retroviral.
3. The lipid particle of embodiment 1 or embodiment 2, wherein the RNA comprises a retroviral packaging sequence that is 3′ to the 5′ LTR.
4. A lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a retroviral packaging sequence; a gag start codon; a RNA sequence encoding a heterologous protein; and a poly-A tail,
wherein each of the R element of the 5′ LTR, the U5 element of the 5′ LTR, and the gag start codon is retroviral.
5. The lipid particle of any of embodiments 1-4, further comprising a U3 element of a 5′ LTR.
6. The lipid particle of any of embodiments 1-5, wherein the RNA comprises a polyadenylation site.
7. The lipid particle of embodiment 6, wherein the RNA comprises a 3′ long terminal repeat (3′ LTR), and the polyadenylation site is located within the 3′ LTR.
8. The lipid particle of any of embodiments 1-7, wherein the RNA comprises a mutated primer binding site (PBS).
9. The lipid particle of any of embodiments 3-8, wherein the retroviral packaging sequence is selected from the group comprising HIV psi, MLV psi, SNV E, or a portion of any thereof.
10. The lipid particle of any of embodiments 3-9, wherein the retroviral packaging sequence comprises stem-loop 1 (SL1) of HIV psi.
11. The lipid particle of any of embodiments 3-10, wherein the retroviral packaging sequence comprises stem-loop 2 (SL2) of HIV psi.
12. The lipid particle of any of embodiments 3-11, wherein the retroviral packaging sequence comprises stem-loop 3 (SL3) of HIV psi.
13. The lipid particle of any of embodiments 3-12, wherein the retroviral packaging sequence comprises stem-loop 4 (SL4) of HIV psi.
14. The lipid particle of any one of embodiments 3-13, wherein the retroviral packaging sequence is HIV psi.
15. The lipid particle of any one of embodiments 3-14, wherein the retroviral packaging sequence comprises a mutation in a major splice donor site.
16. The lipid particle of embodiment 15, wherein the major splice donor site is a major splice donor site contained in SL2 of HIV psi.
17. The lipid particle of embodiment 15 or embodiment 16, wherein the mutation is a mutation that inhibits splicing at the major splice donor site.
18. The lipid particle of any one of embodiments 15-17, wherein the mutation in the major splice donor site comprises a mutation that prevents splicing at the major splice donor site.
19. The lipid particle of any of embodiments 1 and 3-18, wherein the RNA comprises a retroviral sequence having at least about 80% sequence identity to the sequence of a retroviral genome that is about 20, 30, 40, 50, 60, 70, 80, 90, 100, 120, 140, 160, 180, 200, 220, 240, 260, 280, 300, 320, 340, 360, 380, or 400 nucleotides in length and comprises the gag start codon.
20. The lipid particle of any of embodiments 2, 3, and 5-18, wherein the RNA comprises a retroviral sequence having at least about 80% sequence identity to the sequence of a retroviral genome that is about 20, 30, 40, 50, 60, 70, 80, 90, 100, 120, 140, 160, 180, 200, 220, 240, 260, 280, 300, 320, 340, 360, 380, or 400 nucleotides in length and comprises a gag start codon.
21. The lipid particle of embodiment 19 or embodiment 20, wherein the retroviral sequence comprises between about 20-400, between about 40 and about 350, between about 60 and about 300, between about 80 and about 250, or between about 100 and about 200 nucleotides 5′ to the gag start codon.
22. The lipid particle of any of embodiments 19-21, wherein the retroviral sequence comprises between about 20 and about 400, between about 40 and about 350, between about 60 and about 300, between about 80 and about 250, or between about 100 and about 200 nucleotides 3′ to the gag start codon.
23. The lipid particle of any of embodiments 1-22, wherein the lumen comprises a capsid comprising a retroviral capsid protein enclosing the RNA.
24. The lipid particle of embodiment 23, wherein the retroviral capsid protein and the retroviral packaging sequence are capable of associating with each other, optionally wherein the retroviral capsid protein and the retroviral packaging sequence are from the same retroviral species.
25. The lipid particle of any of embodiments 1-24, wherein the lipid particle comprises a retroviral matrix protein.
26. The lipid particle of any of embodiments 1 and 3-25, further comprising a RNA sequence encoding a viral structural protein or a portion thereof, which is located between the gag start codon and the RNA sequence encoding a heterologous protein.
27. The lipid particle of any of embodiments 1 and 3-25, wherein the RNA does not comprise nucleotides between the gag start codon and the RNA sequence encoding a heterologous protein.
28. A lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a RNA sequence encoding a viral structural protein or a portion thereof; a RNA sequence encoding a heterologous protein; and a poly-A tail, wherein each of the R element of the 5′ LTR and the U5 element of the 5′ LTR is retroviral.
29. The lipid particle of embodiment 28, wherein the viral structural protein or a portion thereof is a retroviral structural protein or a portion thereof.
30. The lipid particle of any of embodiments 26, 28, and 29, wherein the RNA comprises a bicistronic element located between the RNA sequence encoding the viral structural protein or a portion thereof and the RNA sequence encoding the heterologous protein.
31. The lipid particle of embodiment 30, wherein the bicistronic element is an internal ribosome entry site (IRES) element or a sequence encoding a 2A self-cleaving peptide.
32. The lipid particle of embodiment 30 or embodiment 31, wherein the bicistronic element is a sequence encoding a 2A self-cleaving peptide, and the 2A self-cleaving peptide is T2A.
33. The lipid particle of embodiment 32, wherein T2A comprises the sequence set forth in SEQ ID NO:76.
34. The lipid particle of any of embodiments 25 and 27-33, wherein the RNA encodes, from a 5′ to 3′ direction: the viral structural protein or portion thereof, T2A, and the heterologous protein.
35. The lipid particle of any of embodiments 26 and 28-34, wherein the viral structural protein is a retroviral gag.
36. The lipid particle of embodiment 35, wherein the RNA sequence encoding the viral structural protein or a portion thereof encodes an N-terminal portion of a retroviral gag.
37. The lipid particle of embodiment 35 or embodiment 36, wherein the RNA sequence encoding the viral structural protein or a portion thereof comprises the sequence set forth in SEQ ID NO: 52.
38. The lipid particle of any of embodiments 1-37, wherein the RNA encodes the sequence set forth in SEQ ID NO:77 and the heterologous protein.
39. The lipid particle of any of embodiments 1-38, wherein the RNA is present as a first genomic viral RNA and the lipid particle further comprises a second genomic viral RNA.
40. The lipid particle of embodiment 39, wherein the first genomic viral RNA and the second viral genomic RNA genome are identical.
41. The lipid particle of embodiment 39, wherein the first genomic viral RNA and the second viral genomic RNA genome are different.
42. The lipid particle of any of embodiments 1-41, wherein the heterologous protein is a first heterologous protein and the lipid particle further comprises one or more additional heterologous protein(s), wherein the lipid particle comprises:
(a) a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}; and/or
(b) a fusion protein comprising a viral matrix (MA) protein and the one or more additional heterologous protein(s).
43. The lipid particle of embodiment 42, wherein the viral MA protein in (a) and/or (b) is derived from human immunodeficiency virus (HIV).
44. The lipid particle of embodiment 42 or embodiment 43, wherein the viral MA protein in (a) and/or (b) comprises the sequence set forth in SEQ ID NO:78.
45. The lipid particle of any of embodiments 42-44, wherein MS2_{cp} in (a) comprises the sequence set forth in SEQ ID NO:79.
46. The lipid particle of any of embodiments 42-45, wherein the fusion protein of (a) comprises the sequence set forth in SEQ ID NO:74.
47. The lipid particle of any one of embodiments 42-45, wherein the fusion protein of (a) comprises:
the amino acid sequence of SEQ ID NOs: 134 or 190; or
the amino acid sequence of SEQ ID NOs: 74 or 191; or
the amino acid sequence encoded by the nucleic acid sequence set forth in SEQ ID NO: 62 or 150
48. The lipid particle of any of embodiments 1-47, wherein the RNA comprises a 5′ cap.
49. The lipid particle of any of embodiments 1-48, wherein the RNA is a self-inactivating lentiviral vector genome.
50. A lipid particle comprising a lipid bilayer enclosing a lumen; a fusion protein comprising a viral matrix (MA) protein and a MS2 coat protein (MS2_{cp}); and an RNA sequence encoding a heterologous protein.
51. The lipid particle of embodiment 50, wherein the RNA sequence encoding a heterologous protein comprises a MS2_{cp}-binding loop.
52. The lipid particle of embodiment 51, wherein the MS2_{cp}-binding loop comprises the RNA sequence transcribed from the nucleic acid set forth in SEQ ID NO: 185 or SEQ ID NO: 174.
53. The lipid particle of embodiment 51 or embodiment 52, wherein the MS2_{cp}-binding loop comprises the RNA sequence set forth in SEQ ID NO: 208.
54. The lipid particle of any one of embodiments 50-53, wherein the RNA sequence encoding a heterologous protein comprises a plurality of MS2_{cp}-binding loops.
55. The lipid particle of embodiment 54, wherein the plurality of MS2_{cp}-binding loops comprises at or a at least 2, 5, 6, 10, 12, 15, 20, or 24 MS2_{cp}-binding loops.
56. The lipid particle of any one of embodiments 50-55, wherein the RNA sequence encoding a heterologous protein comprises a plurality of MS2_{cp}-binding loops comprising between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops.
57. The lipid particle of any one of embodiments 54-56, wherein the plurality of MS2_{cp}-binding loops comprises the RNA sequence transcribed from a nucleic acid sequence selected from the group consisting of SEQ ID NOs: 175-178.
58. A lipid particle comprising a lipid bilayer enclosing a lumen; a viral matrix (MA) protein; an RNA-binding protein; and an RNA sequence encoding a heterologous protein, wherein the RNA-binding protein is incorporated into the lipid particle as a fusion protein with the viral MA protein, and wherein the RNA sequence encoding the heterologous protein comprises a binding site for binding to the RNA-binding protein.
59. The lipid particle of embodiment 58, wherein the RNA sequence encoding a heterologous protein comprises at or at least 2, 5, 6, 10, 12, 15, 20, or 24 binding sites for binding to the RNA-binding protein.
60. The lipid particle of embodiment 58 or embodiment 59, wherein the RNA sequence encoding a heterologous protein comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20, binding sites for binding to the RNA-binding protein.
61. The lipid particle of any one of embodiments 58-60, wherein the RNA-binding protein is MS2 coat protein (MS2_{cp}).
62. The lipid particle of embodiment 61, wherein MS2_{cp} comprises the sequence set forth in SEQ ID NO:79.
63. The lipid particle of any one of embodiments 58-62, wherein the RNA-binding protein is MS2_{cp} and the binding site is an MS2_{cp}-binding loop for binding to the MS2_{cp}.
64. The lipid particle of embodiment 63, wherein the MS2_{cp}-binding loop comprises the RNA sequence transcribed from the nucleic acid set forth in SEQ ID NO: 185 or SEQ ID NO: 174.
65. The lipid particle of embodiment 63 or embodiment 64, wherein the MS2_{cp}-binding loop comprises the RNA sequence set forth in SEQ ID NO: 208.
66. The lipid particle of any one of embodiments 58-65, wherein the RNA sequence encoding a heterologous protein comprises a plurality of MS2_{cp}-binding loops.
67. The lipid particle of embodiment 66, wherein the plurality of MS2_{cp}-binding loops comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops.
68. The lipid particle of embodiment 66 or embodiment 67, wherein the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops.
69. The lipid particle of any one of embodiments 66-68, wherein the plurality of MS2_{cp}-binding loops comprises the RNA sequence transcribed from a nucleic acid sequence selected from the group consisting of SEQ ID NOs: 175-178.
70. The lipid particle of any one of embodiments 58-60, wherein the RNA-binding protein is lambda N protein (λN) or a functional variant thereof.
71. The lipid particle of embodiment 70, wherein the λN or a functional variant thereof comprises the amino acid sequence of SEQ ID NO: 187 or an amino acid sequence having at least 90% or 95% sequence identity to the amino acid sequence of SEQ ID NO: 187.
72. The lipid particle of embodiment 70 or embodiment 71, wherein the λN or a functional variant thereof comprises the amino acid sequence of SEQ ID NO: 187.
73. The lipid particle of embodiment 70, wherein the λN or a functional variant thereof comprises the amino acid sequence of SEQ ID NO: 188 or an amino acid sequence having at least 90% or 95% sequence identity to the amino acid sequence of SEQ ID NO: 188.
74. The lipid particle of embodiment 70 or embodiment 73, wherein the λN or a functional variant thereof comprises the amino acid sequence of SEQ ID NO: 188.
75. The lipid particle of any one of embodiments 58-60 and 70-74, wherein the RNA-binding protein is λN or a functional variant thereof and the binding site is a boxB binding site for binding to the λN or a functional variant thereof.
76. The lipid particle of embodiment 75, wherein the boxB binding site comprises the RNA sequence transcribed from the nucleic acid set forth in SEQ ID NO: 186.
77. The lipid particle of any one of embodiments 58-60 and 70-76, wherein the RNA sequence encoding a heterologous protein comprises a plurality of boxB binding sites.
78. The lipid particle of 77, wherein the plurality of boxB binding sites comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 boxB binding sites.
79. The lipid particle of embodiment 77 or embodiment 78, wherein the plurality of boxB binding sites comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 boxB binding sites.
80. The lipid particle of any one of embodiments 77-79, wherein the plurality of boxB binding sites comprises the RNA sequence transcribed from a nucleic acid sequence selected from the group consisting of SEQ ID NOs: 179-184.
81. A lipid particle comprising a lipid bilayer enclosing a lumen; a viral matrix (MA) protein; a MS2 coat protein (MS2_{cp}); and an RNA sequence encoding a heterologous protein, wherein the MS2_{cp} is incorporated into the lipid particle as a fusion protein with the viral MA protein, and wherein the RNA sequence encoding the heterologous protein comprises a MS2_{cp}-binding loop for binding to MS2_{cp}.
82. The lipid particle of any of embodiments 50-81, wherein the viral MA protein is attached to a portion of the lipid bilayer that is in contact with the lumen.
83. The lipid particle of any of embodiments 50-82, wherein the viral MA protein reversibly binds to the lipid bilayer.
84. The lipid particle of any of embodiments 50-83, wherein the fusion protein comprises, from a 5′ to 3′ direction: the viral MA protein and MS2_{cp}.
85. The lipid particle of any of embodiments 50-84, wherein the RNA sequence encoding a heterologous protein comprises a MS2_{cp}-binding loop, optionally 12 or 24 MS2_{cp}-binding loops.
86. The lipid particle of any of embodiments 50-85, wherein the viral MA protein is derived from human immunodeficiency virus (HIV).
87. The lipid particle of any of embodiments 50-86, wherein the viral MA protein comprises the sequence set forth in SEQ ID NO:78.
88. The lipid particle of any of embodiments 50-87, wherein MS2_{cp} comprises the sequence set forth in SEQ ID NO:79.
89. The lipid particle of any of embodiments 50-88, wherein the fusion protein comprises the sequence set forth in SEQ ID NO:74.
90. The lipid particle of any of embodiments 50-89, further comprising a transfer plasmid encoding a guide RNA (gRNA), optionally a single guide RNA (sgRNA), under the control of a U6 promoter.
91. The lipid particle of any of embodiments 50-90, wherein the heterologous protein is a first heterologous protein and the lipid particle further comprises one or more additional heterologous protein(s), wherein the lipid particle comprises:
(a) a RNA comprising a RNA sequence encoding the one or more additional heterologous protein(s) and a RNA sequence encoding a viral structural protein or a portion thereof; and/or
(b) a fusion protein comprising a viral matrix (MA) protein and the one or more additional heterologous protein(s).
92. The lipid particle of embodiment 91, wherein the viral structural protein is gag.
93. The lipid particle of embodiment 92, wherein the RNA sequence encoding the viral structural protein or a portion thereof in (a) encodes an N-terminal portion of gag.
94. The lipid particle of embodiment 92 or embodiment 93, wherein the RNA sequence encoding the viral structural protein or a portion thereof in (a) comprises the sequence set forth in SEQ ID NO: 52.
95. The lipid particle of any of embodiments 92-94, wherein the viral MA protein in (b) is derived from human immunodeficiency virus (HIV).
96. The lipid particle of any of embodiments 92-95, wherein the viral MA protein in (b) comprises the sequence set forth in SEQ ID NO:78.
97. A lipid particle comprising a lipid bilayer enclosing a lumen a fusion protein comprising a viral matrix (MA) protein and a heterologous protein.
98. A lipid particle comprising a lipid bilayer enclosing a lumen; a viral matrix (MA) protein; and a heterologous protein, wherein the heterologous protein is incorporated into the lipid particle as a fusion protein with the viral MA protein.
99. The lipid particle of embodiment 97 or embodiment 98, wherein the viral MA protein is attached to a portion of the lipid bilayer that is in contact with the lumen.
100. The lipid particle of any of embodiments 97-99, wherein the viral MA protein reversibly binds to the lipid bilayer.
101. The lipid particle of any of embodiments 97-100, wherein the fusion protein comprises, from a 5′ to 3′ direction: the viral MA protein and the heterologous protein.
102. The lipid particle of any of embodiments 97-101, wherein the viral MA protein is derived from human immunodeficiency virus (HIV).
103. The lipid particle of any of embodiments 97-102, wherein the viral MA protein comprises the sequence set forth in SEQ ID NO:78.
104. The lipid particle of any of embodiments 97-103, further comprising a transfer plasmid encoding a guide RNA (gRNA), optionally a single guide RNA (sgRNA), under the control of a U6 promoter.
105. The lipid particle of any of embodiments 97-104, wherein the heterologous protein is a first heterologous protein and the lipid particle further comprises one or more additional heterologous protein(s), wherein the lipid particle comprises:
(a) a RNA comprising a RNA sequence encoding the one or more additional heterologous protein(s) and a RNA sequence encoding a viral structural protein or a portion thereof; and/or
(b) a viral matrix (MA) protein, a MS2 coat protein (MS2_{cp}), and a RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}.
106. The lipid particle of embodiment 105, wherein the viral structural protein in (a) is gag.
107. The lipid particle of embodiment 106, wherein the RNA sequence encoding the viral structural protein or a portion thereof in (a) encodes an N-terminal portion of gag.
108. The lipid particle of embodiment 106 or embodiment 107, wherein the RNA sequence encoding the viral structural protein or a portion thereof in (a) comprises the sequence set forth in SEQ ID NO: 52.
109. The lipid particle of any of embodiments 105-108, wherein the viral MA protein in (b) is derived from HIV.
110. The lipid particle of any of embodiments 105-109, wherein the viral MA protein in (b) comprises the sequence set forth in SEQ ID NO:78.
111. The lipid particle of any of embodiments 105-110, wherein MS2_{cp} in (b) comprises the
112. The lipid particle of any of embodiments 105-111, wherein the fusion protein of (b) comprises the sequence set forth in SEQ ID NO:74.
113. The lipid particle of any of embodiments 105-112, wherein the fusion protein of (b) comprises the sequence set forth in SEQ ID NO: 191.
114. A lipid particle comprising a lipid bilayer enclosing a lumen; a fusion protein comprising a viral envelope glycoprotein and an RNA-binding protein; and an RNA sequence encoding a heterologous protein, wherein the RNA sequence encoding the heterologous protein comprises a binding site for binding to the RNA-binding protein.
115. The lipid particle of embodiment 114, wherein the fusion protein comprises, from an N-terminus to C-terminus direction: the viral envelope glycoprotein and the RNA binding protein.
116. The lipid particle of embodiment 114 or embodiment 115, wherein the RNA-binding protein is fused to the C-terminus of the viral envelope glycoprotein.
117. A lipid particle comprising a lipid bilayer enclosing a lumen; a fusion protein comprising a VSV-G protein or a functional variant thereof and an RNA-binding protein; and an RNA sequence encoding a heterologous protein, wherein the RNA sequence encoding the heterologous protein comprises a binding site for binding to the RNA-binding protein.
118. The lipid particle of embodiment 117, wherein the VSV-G or a functional variant thereof comprises the amino acid sequence of SEQ ID NO: 199, or an amino acid sequence having at least 90%, 91%, 92%, 93%, 94%, 95%, 96%, 97%, 98%, or 99% sequence identity to the amino acid sequence of SEQ ID NO: 199.
119. The lipid particle of embodiment 117 or embodiment 118, wherein the VSV-G or a functional variant thereof comprises the amino acid sequence of SEQ ID NO: 199.
120. The lipid particle of any one of embodiments 114-119, wherein the RNA sequence encoding a heterologous protein comprises at or at least 2, 5, 6, 10, 12, 15, 20, or 24 binding sites for binding to the RNA-binding protein.
121. The lipid particle of any one of embodiments 114-120, wherein the RNA sequence encoding a heterologous protein comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20, binding sites for binding to the RNA-binding protein.
122. The lipid particle of any one of embodiments 114-116, wherein the lipid particle is pseudotyped with the viral envelope glycoprotein.
123. The lipid particle of any one of embodiments 117-121, wherein the fusion protein comprises, from an N-terminus to C-terminus direction: the VSV-G protein or a functional variant thereof and the RNA binding protein.
124. The lipid particle of any one of embodiments 117-121, wherein the RNA-binding protein is fused to the C-terminus of the VSV-G protein or a functional variant thereof.
125. The lipid particle of any one of embodiments 114-124, wherein the RNA-binding protein is MS2 coat protein (MS2_{cp}).
126. The lipid particle of embodiment 125, wherein MS2_{cp} comprises the sequence set forth in SEQ ID NO: 79.
127. The lipid particle of embodiment 125 or embodiment 126, wherein the MS2_{cp} is a homodimer.
128. The lipid particle of embodiment 125 or embodiment 126, wherein the MS2_{cp} is a tandem dimer.
129. The lipid particle of any one of embodiments 114-128, wherein the binding site is an MS2_{cp}-binding loop for binding to the MS2_{cp}.
130. The lipid particle of any one of embodiments 114-129, wherein the RNA sequence encoding a heterologous protein comprises a plurality of MS2_{cp}-binding loops for binding to the MS2_{cp}.
131. The lipid particle of embodiment 130, wherein the plurality of MS2_{cp}-binding loops comprises at or at least 2, 4, 6, 8, 10, 12, 14, 16, 18, 20, 22, or 24 MS2_{cp}-binding loops.
132. The lipid particle of embodiment 130 or embodiment 131, wherein the plurality of MS2_{cp}-binding loops comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops.
133. The lipid particle of any one of embodiments 130-132, wherein the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops.
134. The lipid particle of any one of embodiments 114-124, wherein the RNA-binding protein is lambda N protein (λN) or a functional variant thereof.
135. The lipid particle of embodiment 134, wherein the λN or a functional variant thereof comprises the amino acid sequence of SEQ ID NO: 187 or an amino acid sequence having at least 90% or 95% sequence identity to the amino acid sequence of SEQ ID NO: 187.
136. The lipid particle of embodiment 134 or embodiment 135, wherein the λN or a functional variant thereof comprises the amino acid sequence of SEQ ID NO: 187.
137. The lipid particle of embodiment 134, wherein the λN or a functional variant thereof comprises the amino acid sequence of SEQ ID NO: 188 or an amino acid sequence having at least 90% or 95% sequence identity to the amino acid sequence of SEQ ID NO: 188.
138. The lipid particle of embodiment 134, wherein the λN or a functional variant thereof comprises the amino acid sequence of SEQ ID NO: 188.
139. The lipid particle of any one of embodiments 114-125 and 134-138, wherein the RNA-binding protein is λN or a functional variant thereof and the binding site is a boxB binding site for binding to the λN or a functional variant thereof.
140. The lipid particle of any one of embodiments 114-124 and 134-139, wherein the RNA sequence encoding a heterologous protein comprises a plurality of boxB binding sites.
141. The lipid particle of any one of embodiments 140, wherein the plurality of boxB binding sites comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 boxB binding sites.
142. The lipid particle of embodiment 140 or embodiment 141, wherein the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops.
143. The lipid particle of any one of embodiments 140-142, wherein the plurality of boxB binding sites comprises the RNA sequence transcribed from a nucleic acid sequence selected from the group consisting of SEQ ID NOs: 179-184.
144. The lipid particle of any of embodiments 1-143, wherein the heterologous protein is a genome-modifying protein.
145. The lipid particle of embodiment 144, wherein the genome-modifying protein comprises a recombinant nuclease, a nickase, an integrase, reverse transcriptase, or a combination thereof.
146. The lipid particle of embodiment 144 or embodiment 145, wherein the genome-modifying protein comprises a zinc-finger nuclease (ZFN), a transcription-activator like effector nucleases (TALEN), or a CRISPR-associated (Cas) protein.
147. The lipid particle of any of embodiments 144-146, wherein the genome-modifying protein is a Cas protein.
148. The lipid particle of any of embodiments 144-147, wherein the genome-modifying protein is (i) Cas9, optionally saCas9 or spCas9; or (ii) cpf1.
149. The lipid particle of any of embodiments 1-148, further comprising a guide RNA (gRNA) in the lumen.
150. The lipid particle of embodiment 149, wherein the gRNA is a single guide RNA (sgRNA).
151. The lipid particle of any of embodiments 1-150, wherein the lipid particle is pseudotyped with a viral envelope glycoprotein.
152. The lipid particle of embodiment 151, wherein the viral envelope glycoprotein is a VSV-G protein or a functional variant thereof.
153. The lipid particle of embodiment 152, wherein the VSV-G or a functional variant thereof comprises the amino acid sequence of SEQ ID NO: 199, or an amino acid sequence having at least 90%, 91%, 92%, 93%, 94%, 95%, 96%, 97%, 98%, or 99% sequence identity to the amino acid sequence of SEQ ID NO: 199.
154. The lipid particle of embodiment 152 or embodiment 153, wherein the VSV-G or a functional variant thereof comprises the amino acid sequence of SEQ ID NO: 199.
155. The lipid particle of embodiment 151, wherein the viral envelope glycoprotein is a Cocal virus G protein or a functional variant thereof.
156. The lipid particle of embodiment 151, wherein the viral envelope glycoprotein is an Alphavirus fusion protein (e.g. Sindbis virus) or a functional variant thereof.
157. The lipid particle of embodiment 151, wherein the viral envelope glycoprotein is a Paramyxoviridae fusion protein (e.g., a Morbillivirus or a Henipavirus) or a functional variant thereof.
158. The lipid particle of embodiment 151 or embodiment 157, wherein the viral envelope glycoprotein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof.
159. The lipid particle of embodiment 151 or embodiment 157, wherein the viral envelope glycoprotein is a Henipavirus fusion protein (e.g., Nipah virus, Hendra virus, Cedar virus, Kumasi virus, Mòjiāng virus, Langya virus) or a functional variant thereof.
160. The lipid particle of any of embodiments 151-159, wherein the viral envelope glycoprotein comprises one or more modifications to reduce binding to its native receptor.
161. The lipid particle of any of embodiments 151, 157, 159, and 160, wherein the viral envelope glycoprotein comprises a Nipah virus F glycoprotein (NiV-F) or a biologically active portion thereof and a Nipah virus G glycoprotein (NiV-G) or a biologically active portion thereof.
162. The lipid particle of embodiment 161, wherein the Niv-F or a biologically active portion thereof comprises the amino acid sequence set forth in SEQ ID NO: 145, or an amino acid sequence having at least 90%, 91%, 92%, 93%, 94%, 95%, 96%, 97%, 98%, or 99% sequence identity to the amino acid sequence set forth in SEQ ID NO: 145; and/or the Niv-G or a biologically active portion thereof comprises the amino acid sequence set forth in SEQ ID NO: 147, or an amino acid sequence having at least 90%, 91%, 92%, 93%, 94%, 95%, 96%, 97%, 98%, or 99% sequence identity to the amino acid sequence set forth in SEQ ID NO: 147.
163. The lipid particle of embodiment 161, wherein the Niv-F or a biologically active portion thereof comprises the amino acid sequence set forth in SEQ ID NO: 145; and/or the Niv-G or a biologically active portion thereof comprises the amino acid sequence set forth in SEQ ID NO: 147, 164. The lipid particle of embodiment 161, wherein the NiV-G or the biologically active portion thereof is a wild-type NiV-G protein or a functionally active variant or biologically active portion thereof.
165. The lipid particle of embodiment 161 or embodiment 164, wherein the NiV-G protein or the biologically active portion is truncated and lacks up to 40 contiguous amino acid residues at or near the N-terminus of the wild-type NiV-G protein.
166. The lipid particle of any of embodiments 161-165, wherein the NiV-G protein or the biologically active portion has a 5 amino acid truncation at or near the N-terminus of the wild-type NiV-G protein, optionally wherein the NiV-G protein or the biologically active portion thereof has the amino acid sequence set forth in SEQ ID NO:12, or a sequence of amino acids that exhibits at least at or about 80%, 85%, 90% or 95% sequence identity to the sequence set forth in SEQ ID NO:12.
167. The lipid particle of any of embodiments 161-166, wherein the NiV-G protein or the biologically active portion has a 10 amino acid truncation at or near the N-terminus of the wild-type NiV-G protein, optionally wherein the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44.
168. The lipid particle of any of embodiments 161-167, wherein the NiV-G protein or the biologically active portion has a 15 amino acid truncation at or near the N-terminus of the wild-type NiV-G protein, optionally wherein the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45.
169. The lipid particle of any of embodiments 161-168, wherein the NiV-G protein or the biologically active portion has a 20 amino acid truncation at or near the N-terminus of the wild-type NiV-G protein, optionally wherein the NiV-G protein or the biologically active portion thereof has the amino acid sequence set forth in SEQ ID NO: 13, or a sequence of amino acids that exhibits at least at or about 80%, 85%, 90% or 95% sequence identity to the sequence set forth in SEQ ID NO:13.
170. The lipid particle of any of embodiments 161-168, wherein the NiV-G protein or the biologically active portion has a 25 amino acid truncation at or near the N-terminus of the wild-type NiV-G protein, optionally wherein the NiV-G protein or the biologically active portion thereof has the amino acid sequence set forth in SEQ ID NO: 14, or a sequence of amino acids that exhibits at least at or about 80%, 85%, 90% or 95% sequence identity to the sequence set forth in SEQ ID NO: 14.
171. The lipid particle of any of embodiments 161-168, wherein the NiV-G protein or the biologically active portion has a 30 amino acid truncation at or near the N-terminus of the wild-type NiV-G protein, optionally wherein the NiV-G protein or the biologically active portion thereof has the amino acid sequence set forth in SEQ ID NO:43, or a sequence of amino acids that exhibits at least at or about 80%, 85%, 90% or 95% sequence identity to the sequence set forth in SEQ ID NO:43.
172. The lipid particle of any of embodiments 161-168, wherein the NiV-G protein or the biologically active portion has a 34 amino acid truncation at or near the N-terminus of the wild-type NiV-G protein, optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.
173. The lipid particle of any of embodiments 161-172, wherein the NiV-G-protein or the biologically active portion thereof is a mutant NiV-G protein that exhibits reduced binding to Ephrin B2 or Ephrin B3.
174. The lipid particle of embodiment 173, wherein the mutant NiV-G protein or the biologically active portion comprises one or more amino acid substitutions corresponding to amino acid substitutions selected from the group consisting of E501A, W504A, Q530A and E533A with reference to numbering set forth in SEQ ID NO:4.
175. The lipid particle of embodiment 173 or embodiment 174, wherein the mutant NiV-G protein or the biologically active portion comprises the amino acid sequence set forth in SEQ ID NO: 17 or a sequence of amino acids that exhibits at least at or about 80%, 85%, 90% or 95% sequence identity to the sequence set forth in SEQ ID NO: 17.
176. The lipid particle of embodiment 173 or embodiment 174, wherein the NiV-G protein or the biologically active portion has the amino acid sequence set forth in SEQ ID NO: 18 or a sequence of amino acids that exhibits at least at or about 80%, 85%, 90% or 95% sequence identity to the sequence set forth in SEQ ID NO: 18.
177. The lipid particle of any of embodiments 161-176, wherein the NiV-F protein or the biologically active portion thereof is a wild-type NiV-F protein or is a functionally active variant or a biologically active portion thereof.
178. The lipid particle of any of embodiments 161-177, wherein the NiV-F protein or the biologically active portion thereof has a 20 amino acid truncation at or near the C-terminus of the wild-type NiV-F protein, optionally wherein the NiV-F protein or the biologically active portion thereof comprises the sequence set forth in SEQ ID NO: 20 or a sequence of amino acids that exhibits at least at or about 80%, 85%, 90% or 95% sequence identity to the sequence set forth in SEQ ID NO: 20.
179. The lipid particle of any of embodiments 161-178, wherein the NiV-F protein or the biologically active portion thereof comprises:
   i) a 20 amino acid truncation at or near the C-terminus of the wild-type NiV-F protein; and
   ii) a point mutation on an N-linked glycosylation site,
   optionally wherein the NiV-F protein or the biologically active portion thereof comprises the sequence set forth in SEQ ID NO: 15, or a sequence of amino acids that exhibits at least at or about 80%, 85%, 90% or 95% sequence identity to the sequence set forth in SEQ ID NO: 15.
180. The lipid particle of any of embodiments 161-179, wherein the NiV-F protein or the biologically active portion thereof has a 22 amino acid truncation at or near the C-terminus of the wild-type NiV-F protein, optionally wherein the NiV-F protein or the biologically active portion thereof comprises the sequence set forth in SEQ ID NO: 16, 19, or 21 or a sequence of amino acids that exhibits at least at or about 80%, 85%, 90% or 95% sequence identity to the sequence set forth in SEQ ID NO: 16, 19, or 21.
181. The lipid particle of any of embodiments 161-177 and 180, wherein the NiV-F protein or the biologically active portion thereof comprises the amino acid sequence set forth in SEQ ID NO:21, or a sequence of amino acids that exhibits at least at or about 80%, 85%, 90% or 95% sequence identity to the sequence set forth in SEQ ID NO:21.
182. The lipid particle of any of embodiments 161-177, 180, and 181, wherein the Niv-G protein comprises the amino acid sequence set forth in SEQ ID NO: 17, and the Niv-F protein comprises the amino acid sequence set forth in SEQ ID NO:21.
183. The lipid particle of any of embodiments 1-182, further comprising a targeting moiety.
184. The lipid particle of embodiment 183, wherein the targeting moiety is selected from the group consisting of a CD3-binding agent, a CD8-binding agent, and a CD4-binding agent.
185. The lipid particle of embodiment 183 or embodiment 184, wherein the targeting moiety is a CD3-binding agent, optionally an anti-CD3 antibody or an antigen-binding fragment.
186. The lipid particle of embodiment 183 or embodiment 184, wherein the targeting moiety is a CD8-binding agent, optionally an anti-CD8 antibody or an antigen-binding fragment.
187. The lipid particle of embodiment 183 or embodiment 184, wherein the targeting moiety is a CD4-binding agent, optionally an anti-CD4 antibody or an antigen-binding fragment.
188. The lipid particle of any of embodiments 183-187, wherein the targeting moiety is exposed on the surface of the lipid particle.
189. The lipid particle of any of embodiments 183-188, wherein the targeting moiety is fused to a transmembrane domain incorporated into the bilayer of the lipid particle.
190. The lipid particle of any of embodiments 1-189, wherein the lipid particle is a retroviral vector or a retroviral-like particle.
191. The lipid particle of any of embodiments 1-190, wherein the retroviral vector or the retroviral-like particle is replication-deficient.
192. The lipid particle of any of embodiments 1-191, where the lipid particle does not comprise reverse transcriptase or does not comprise reverse transcriptase activity.
193. The lipid particle of any of embodiments 1-191, where the lipid particle does not comprise a protein with reverse transcriptase activity.
194. The lipid particle of embodiment 192 or embodiment 193, wherein the lipid particle does not comprise reverse transcriptase.
195. The lipid particle of embodiment 192 or embodiment 193, wherein the lipid particle comprises non-functional reverse transcriptase, optionally wherein the reverse transcriptase is mutated.
196. The lipid particle of any of embodiments 190-195, wherein the retroviral vector or retroviral-like particle comprises a RNA that is a self-inactivating lentiviral vector genome.
197. The lipid particle of any of embodiments 191-196, wherein the retroviral vector or retroviral-like particle comprises a RNA comprising a 3′LTR, and the 3′ LTR does not comprise a functional U3 domain, optionally wherein the U3 domain comprises a deletion.
198. The lipid particle of any of embodiments 1-197, wherein the lipid particle is a retroviral particle, and the retroviral particle is a lentiviral particle.
199. The lipid particle of any of embodiments 1-197, wherein the lipid particle is a retrovirus-like particle (VLP).
200. The lipid particle of any of embodiments 1-199, wherein the lipid bilayer is derived from a host cell.
201. The lipid particle of embodiment 200, wherein the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.
202. A method of producing a lipid particle comprising a lipid bilayer enclosing a lumen and a viral ribonucleic acid (RNA), comprising:
(1) providing a host cell comprising (a) a nucleic acid sequence selected from the group consisting of: a 5′ long terminal repeat (5′ LTR); a psi packaging signal sequence; a gag start codon; a RNA sequence encoding a heterologous protein; a 3′ long terminal repeat (3′ LTR); or a combination thereof; and (b) a nucleic acid sequence encoding a protein selected from the group consisting of gag, pol, rev, tat, a viral envelope glycoprotein, or a combination thereof; and
(2) culturing the host cell under conditions to induce packaging of the lipid particle. 203. The method of embodiment 202, further comprising a RNA sequence encoding a viral structural protein or a portion thereof, which is located between the gag start codon and the RNA sequence encoding a heterologous protein.
204. The method of embodiment 202, wherein the gag start codon and the RNA sequence encoding a heterologous protein are part of the same RNA, and the RNA does not comprise nucleotides between the gag start codon and the RNA sequence encoding a heterologous protein.
205. A method of producing a lipid particle comprising a lipid bilayer enclosing a lumen and viral ribonucleic acid (RNA), comprising:
(1) providing a host cell comprising (a) a RNA sequence encoding a heterologous protein and a viral structural protein or a portion thereof; and (b) a nucleic acid sequence encoding a protein selected from the group consisting of gag, pol, rev, tat, a viral envelope glycoprotein, or a combination thereof; and
(2) culturing the host cell under conditions to induce packaging of the lipid particle.
206. The method of embodiment 205, wherein the RNA sequence encoding the viral structural protein or portion thereof is located 5′ to the RNA sequence encoding the heterologous protein.
207. The method of any of embodiments 203, 205, and 206, wherein a bicistronic element is located between the RNA sequence encoding the viral structural protein or portion thereof and the RNA sequence encoding the heterologous protein.
208. The method of embodiment 207, wherein the bicistronic element is an internal ribosome entry site (IRES) element or a sequence encoding a 2A self-cleaving peptide.
209. The method of embodiment 207 or embodiment 208, wherein the bicistronic element is a sequence encoding a 2A self-cleaving peptide, and the 2A self-cleaving peptide is T2A.
210. The method of embodiment 209, wherein T2A comprises the sequence set forth in SEQ ID NO: 76.
211. The method of any of embodiments 203 and 205-210, wherein the RNA sequence encodes, from a 5′ to 3′ direction: the viral structural protein or portion thereof, T2A, and the heterologous protein.
212. The method of any of embodiments 203 and 205-211, wherein the viral structural protein is gag.
213. The method of embodiment 212, wherein the RNA sequence encoding the viral structural protein or a portion thereof encodes an N-terminal portion of gag.
214. The method of embodiment 212 or embodiment 213, wherein the RNA sequence encoding the viral structural protein or a portion thereof comprises the sequence set forth in SEQ ID NO: 52.
215. The method of any of embodiments 202-214, wherein the host cell comprises a nucleic acid sequence that comprises the sequence set forth in SEQ ID NO:77 and encodes the heterologous protein.
216. A method of producing a lipid particle comprising a lipid bilayer enclosing a lumen and a viral transfer plasmid, comprising
(1) providing a host cell comprising (a) a nucleic acid sequence encoding a fusion protein comprising a viral matrix (MA) protein and an RNA binding protein; (b) a nucleic acid sequence encoding a heterologous protein; and (c) a nucleic acid sequence encoding a protein selected from the group consisting of gag, pol, Rev, Tat, a viral envelope glycoprotein, or a combination thereof; and
(2) culturing the host cell under conditions to induce packaging of the lipid particle.
217. The method of embodiment 216, wherein the RNA binding protein is a MS2 coat protein (MS2_{cp}).
218. The method of embodiment 217, wherein MS2_{cp} comprises the sequence set forth in SEQ ID NO:79.
219. The method of any one of embodiments 216-218, wherein the nucleic acid sequence encoding a heterologous protein comprises a MS2_{cp}-binding loop, optionally at or at least 2, 3, 4, 5, 6, 7, 8, 9, 10, 11, 12, 13, 14, 15, 16, 17, 18, 19, 20, 21, 22, 23, or 24 MS2_{cp}-binding loops.
220. The method of embodiment 219, wherein the MS2_{cp}-binding loop comprises the RNA sequence transcribed from the nucleic acid set forth in SEQ ID NO: 185 or SEQ ID NO: 174.
221. The method of embodiment 219 or embodiment 220, wherein the MS2_{cp}-binding loop comprises the RNA sequence set forth in SEQ ID NO: 208.
222. The method of any one of embodiments 216-221, wherein the nucleic acid sequence encoding a heterologous protein comprises a plurality of MS2_{cp}-binding loops, and wherein the plurality of MS2_{cp}-binding loops comprises the RNA sequence transcribed from a nucleic acid sequence selected from the group consisting of SEQ ID NOs: 175-178.
223. The method of embodiment 216, wherein the RNA binding protein is a lambda N protein (λN) or a functional variant thereof.
224. The method of embodiment 223, wherein the λN or a functional variant thereof comprises the amino acid sequence of SEQ ID NO: 187 or an amino acid sequence having at least 90% or 95% sequence identity to the amino acid sequence of SEQ ID NO: 187.
225. The method of embodiment 223 or embodiment 224, wherein the λN or a functional variant thereof comprises the amino acid sequence of SEQ ID NO: 187.
226. The method of embodiment 223, wherein the λN or a functional variant thereof comprises the amino acid sequence of SEQ ID NO: 188 or an amino acid sequence having at least 90% or 95% sequence identity to the amino acid sequence of SEQ ID NO: 188.
227. The method of embodiment 223 or embodiment 226, wherein the λN or a functional variant thereof comprises the amino acid sequence of SEQ ID NO: 188.
228. The method of any one of embodiments 216 and 223-227, wherein the nucleic acid sequence encoding a heterologous protein comprises a boxB binding site for binding to λN or a functional variant thereof, optionally at or at least 2, 3, 4, 5, 6, 7, 8, 9, 10, 11, 12, 13, 14, 15, 16, 17, 18, 19, 20, 21, 22, 23, or 24 boxB binding sites.
229. The method of embodiment 228, wherein the boxB binding site comprises the RNA sequence transcribed from the nucleic acid set forth in SEQ ID NO: 186.
230. The lipid particle of any one of embodiments 216 and 223-229, wherein the nucleic acid sequence encoding a heterologous protein comprises a plurality of boxB binding sites for binding to λN or a functional variant thereof, and the plurality of boxB binding sites comprises the RNA sequence transcribed from a nucleic acid sequence selected from the group consisting of SEQ ID NOs: 179-184.
231. A method of producing a lipid particle comprising a lipid bilayer enclosing a lumen and a viral transfer plasmid, comprising:
(1) providing a host cell comprising (a) a nucleic acid sequence encoding a fusion protein comprising a viral matrix (MA) protein and a MS2 coat protein (MS2_{cp}); (b) a nucleic acid sequence encoding a heterologous protein; and (c) a nucleic acid sequence encoding a protein selected from the group consisting of gag, pol, Rev, Tat, a viral envelope glycoprotein, or a combination thereof; and
(2) culturing the host cell under conditions to induce packaging of the lipid particle.
232. The method of embodiment 231, wherein the fusion protein comprises, from a 5′ to 3′ direction: the viral MA protein and MS2_{cp}.
233. The method of embodiment 231 or embodiment 232, wherein the nucleic acid sequence encoding a heterologous protein comprises a MS2_{cp}-binding loop, optionally 12 or 24 MS2_{cp}-binding loops.
234. The method of any of embodiments 231-233, wherein the viral MA protein is derived from human immunodeficiency virus (HIV).
235. The method of any of embodiments 231-234, wherein the viral MA protein comprises the sequence set forth in SEQ ID NO:78.
236. The method of any of embodiments 231-235, wherein MS2_{cp} comprises the sequence set forth in SEQ ID NO:79.
237. The method of any of embodiments 231-236, wherein the fusion protein comprises the sequence set forth in SEQ ID NO:74.
238. A method of producing a lipid particle comprising a lipid bilayer enclosing a lumen and a viral transfer plasmid, comprising:
(1) providing a host cell comprising (a) a nucleic acid sequence encoding a fusion protein comprising a viral envelope glycoprotein and an RNA binding protein; (b) a nucleic acid sequence encoding a heterologous protein; and
(2) culturing the host cell under conditions to induce packaging of the lipid particle.
239. The method of embodiment 238, wherein the host cells further comprises a nucleic acid sequence encoding a protein selected from the group consisting of gag, pol, Rev, Tat, or a combination thereof 0.240. The method of embodiment 238 or embodiment 239, wherein the fusion protein comprises, from a 5′ to 3′ direction: the viral envelope glycoprotein and the RNA binding protein.
241. The method of any one of embodiments 238-240, wherein the viral envelope glycoprotein is a VSV-G protein or a functional variant thereof.
242. The method of any one of embodiments 238-241, wherein the RNA binding protein is a MS2 coat protein (MS2_{cp}).
243. The method of embodiment 242, wherein MS2_{cp} comprises the sequence set forth in SEQ ID NO:79.
244. The method of any one of embodiments 238-243, wherein the nucleic acid sequence encoding a heterologous protein comprises a MS2_{cp}-binding loop, optionally at or at least 12 or 24 MS2_{cp}-binding loops.
245. The method of embodiment 244, wherein the MS2_{cp}-binding loop comprises the RNA sequence transcribed from the nucleic acid set forth in SEQ ID NO: 185 or SEQ ID NO: 174.
246. The lipid particle of embodiment 244 or embodiment 245, wherein the MS2_{cp}-binding loop comprises the RNA sequence set forth in SEQ ID NO: 208.
247. The method of any one of embodiments 238-246, wherein the nucleic acid sequence encoding a heterologous protein comprises a plurality of MS2_{cp}-binding loops, and wherein the plurality of MS2_{cp}-binding loops comprises the RNA sequence transcribed from a nucleic acid sequence selected from the group consisting of SEQ ID NOs: 175-178.
248. The method of any of embodiments 238-247, wherein the viral envelope glycoprotein is derived from human immunodeficiency virus (HIV).
249. The method of any one of embodiments 238-241, wherein the RNA binding protein is a lambda N protein (λN) or a functional variant thereof.
250. The method of any one of embodiments 238-241 and 249, wherein the nucleic acid sequence encoding a heterologous protein comprises a boxB binding site for binding to λN or a functional variant thereof, optionally at or at least 2, 3, 4, 5, 6, 7, 8, 9, 10, 11, 12, 13, 14, 15, 16, 17, 18, 19, 20, 21, 22, 23, or 24 boxB binding sites.
251. The method of embodiment 250, wherein the boxB binding site comprises the RNA sequence transcribed from the nucleic acid set forth in SEQ ID NO: 186.
252. The lipid particle of any one of embodiments 238-241, 250, and 251, wherein the nucleic acid sequence encoding a heterologous protein comprises a plurality of boxB binding sites for binding to λN or a functional variant thereof, and the plurality of boxB binding sites comprises the RNA sequence transcribed from a nucleic acid sequence selected from the group consisting of SEQ ID NOs: 179-184.
253. A method of producing a lipid particle comprising a lipid bilayer enclosing a lumen and a viral transfer plasmid, comprising:
(1) providing a host cell comprising (a) a nucleic acid sequence encoding a fusion protein comprising viral matrix (MA) protein and a heterologous protein; and (b) a nucleic acid sequence encoding a protein selected from the group consisting of gag, pol, Rev, Tat, a viral envelope glycoprotein, or a combination thereof; and
(2) culturing the host cell under conditions to induce packaging of the lipid particle.
254. The method of embodiment 253, wherein the fusion protein comprises, from a 5′ to 3′ direction: the viral MA protein and the heterologous protein.
255. The method of embodiment 253 or embodiment 254, wherein the viral MA protein is derived from human immunodeficiency virus (HIV).
256. The method of any of embodiments 253-255, wherein the viral MA protein comprises the sequence set forth in SEQ ID NO:78.
257. The method of any of embodiments 202-256, wherein the viral envelope glycoprotein is VSV-G.
258. The method of any of embodiments 202-257, wherein the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.
259. The method of any of embodiments 202-258, wherein the nucleic acid sequence in (b) comprises a 5′ promoter.
260. The method of any of embodiments 231-259, wherein the nucleic acid sequence in (c) comprises a 5′ promoter.
261. The method of embodiment 259 or embodiment 260, wherein the promoter is a cytomegalovirus (CMV) promoter.
262. A lipid particle produced by the methods of any of embodiments 202-261.
263. A composition comprising the lipid particle of any of embodiments 1-201 and 262.
264. A method of introducing a heterologous protein into a target cell, the method comprising contacting the target cell with the lipid particle of any of embodiments 1-201 and 262 or the composition of embodiment 263.
265. A method of genetically engineering a target cell, the method comprising contacting the target cell with the lipid particle of any of embodiments 1-201 and 262 or the composition of embodiment 263.
266. The method of embodiment 264 or embodiment 265, wherein the contacting is in vitro or ex vivo.
267. The method of embodiment 264 or embodiment 265, wherein the contacting is in vivo. 268. A deoxyribonucleic acid (DNA) sequence encoding a gag start codon and a heterologous protein.
269. The DNA sequence of embodiment 268, further encoding a viral structural protein or a portion thereof, wherein the portion of the DNA sequence encoding the viral structural protein is located between the portions of the DNA sequence encoding the gag start codon and the heterologous protein.
270. The DNA sequence of embodiment 268 or 269, further encoding a bicistronic element, wherein the portion of the DNA sequence encoding the bicistronic element is located between the portions of the DNA sequence encoding the viral structural protein or a portion thereof and the heterologous protein.
271. The DNA sequence of embodiment 270, wherein the bicistronic element is an internal ribosome entry site (IRES) element or a sequence encoding a 2A self-cleaving peptide.
272. The DNA sequence of embodiment 271, wherein the 2A self-cleaving peptide is T2A. 273. The DNA sequence of embodiment 272, wherein T2A comprises the sequence set forth in SEQ ID NO:76.
274. The DNA sequence of any of embodiments 269-273, wherein the DNA sequence encodes from a 5′ to 3′ direction: the viral structural protein or portion thereof, T2A, and the heterologous protein.
275. The DNA sequence of any of embodiments 269-274, wherein the viral structural protein is gag.
276. The DNA sequence of embodiment 275, encoding an N-terminal portion of gag.
277. The DNA sequence of embodiment 275 or embodiment 276, wherein the N-terminal portion of gag comprises the sequence set forth in SEQ ID NO:52.
278. The DNA sequence of any of embodiments 268-277, which encodes the sequence set forth in SEQ ID NO:77 and the heterologous protein.
279. The DNA sequence of embodiment 278, which does not comprise nucleotides between the encoded gag start codon and the encoded heterologous protein.
280. The DNA sequence of any of embodiments 268-279, comprising a promoter.
281. The DNA sequence of embodiment 280, wherein the promoter is a cytomegalovirus (CMV) promoter.
282. A DNA sequence encoding a viral matrix (MA) protein, an RNA binding protein, and a cleavage site between the portions of the DNA sequence encoding the MA protein and the RNA binding protein.
283. The DNA sequence of embodiment 282, which encodes a fusion protein comprising, from 5′ to 3′, the viral MA protein and RNA binding protein.
284. The DNA sequence of embodiment 282 or embodiment 283, wherein the RNA binding protein is a MS2 coat protein (MS2_{cp}).
285. The DNA sequence of embodiment 284, wherein MS2_{cp} comprises the sequence set forth in SEQ ID NO:79.
286. The DNA sequence of embodiment 282 or embodiment 283, wherein the RNA binding protein is a lambda N protein (λN) or a functional variant thereof.
287. The DNA sequence of embodiment 286, wherein the λN or a functional variant thereof comprises the amino acid sequence of SEQ ID NO: 187 or 188.
288. The DNA sequence of any of embodiments 282-287 wherein the encoded viral MA protein comprises the sequence set forth in SEQ ID NO:78.
289. The DNA sequence of any one of embodiments 282-288, comprising the nucleic acid sequence set forth in any one of SEQ ID NOs: 62, 150, 153, and 154.
290. A DNA sequence encoding a viral matrix (MA) protein, a MS2 coat protein (MS2_{cp}), and a cleavage site between the portions of the DNA sequence encoding the MA protein and the MS2_{cp}.
291. The DNA sequence of embodiment 290, which encodes a fusion protein comprising, from 5′ to 3′, the viral MA protein and MS2_{cp}.
292. The DNA sequence polynucleotide of embodiment 290 or embodiment 291, wherein the encoded MS2_{cp} comprises a MS2_{cp}-binding loop, optionally 12 or 24 MS2_{cp}-binding loops.
293. The DNA sequence of any of embodiments 290-292, wherein the encoded viral MA protein is derived from human immunodeficiency virus (HIV).
294. The DNA sequence of any of embodiments 290-293, wherein the encoded viral MA protein comprises the sequence set forth in SEQ ID NO:78.
295. The DNA sequence of any of embodiments 290-294, wherein the encoded MS2_{cp} comprises the sequence set forth in SEQ ID NO:79.
296. The DNA sequence of any of embodiments 290-295, wherein the encoded fusion protein comprises the sequence set forth in SEQ ID NO:74.
297. A DNA sequence encoding a viral envelope glycoprotein, an RNA binding protein, and a cleavage site between the portions of the DNA sequence encoding the viral envelope glycoprotein and the RNA binding protein.
298. The DNA sequence of embodiment 297, wherein the fusion protein comprises, from a 5′ to 3′ direction: the viral envelope glycoprotein and the RNA binding protein.
299. The DNA sequence of embodiment 297 or embodiment 298, wherein the viral envelope glycoprotein is a VSV-G protein or a functional variant thereof.
300. The DNA sequence of any of embodiments 297-299, wherein the viral envelope glycoprotein is derived from human immunodeficiency virus (HIV).
301. The DNA sequence of any one of embodiments 297-300, wherein the RNA binding protein is a MS2 coat protein (MS2_{cp}).
302. The DNA sequence of embodiment 301, wherein MS2_{cp} comprises the sequence set forth in SEQ ID NO:79.
303. The DNA sequence of any one of embodiments 297-300, wherein the RNA binding protein is a lambda N protein (λN) or a functional variant thereof.
304. The DNA sequence of embodiment 303, wherein the λN or a functional variant thereof comprises the amino acid sequence set forth in SEQ ID NO: 187 or 188.
305. The DNA sequence of embodiment 297 or embodiment 298, comprising a nucleic acid sequence selected from the group consisting of SEQ ID NOs: 62 and 150-156.
306. The DNA sequence of any one of embodiments 297-302, comprising the nucleic acid sequence set forth in SEQ ID NO: 151 or 152.
307. The DNA sequence of any one of embodiments 297-302, comprising a nucleic acid sequence encoding the amino acid sequence set forth in SEQ ID NO: 157 or 158.
308. The DNA sequence of any one of embodiments 297-302, comprising a nucleic acid sequence encoding the amino acid sequence set forth in SEQ ID NO: 192 or 193.
309. The DNA sequence of any one of embodiments 297-300, 303, and 304, comprising the nucleic acid sequence set forth in SEQ ID NO: 155 or 156.
310. The DNA sequence of any one of embodiments 297-300, 303, 304, and 309, comprising a nucleic acid sequence encoding the amino acid sequence set forth in SEQ ID NO: 161 or 162.
311. The DNA sequence of any one of embodiments 297-300, 303, 304, and 309, comprising a nucleic acid sequence encoding the amino acid sequence set forth in SEQ ID NO: 196 or 197.312.
A DNA sequence encoding a viral matrix (MA) protein and a heterologous protein.
313. The DNA sequence of embodiment 312, which encodes a fusion protein comprising, from 5′ to 3′, the viral MA protein and the heterologous protein.
314. The DNA sequence of embodiment 312 or embodiment 313, wherein the encoded viral MA protein is derived from human immunodeficiency virus (HIV).
315 The DNA sequence of any of embodiments 312-314, wherein the encoded viral MA protein comprises the sequence set forth in SEQ ID NO:78.
316. The lipid particle of any of embodiments 1-201 and 262, the composition of embodiment 263, the method of any of embodiments 202-261 and 264-267, or the DNA sequence of any of embodiments 268-281 and 312-315, wherein the heterologous protein is a genome-modifying protein.
317. The lipid particle, composition, method, or DNA sequence of embodiment 316, wherein the genome-modifying protein comprises a recombinant nuclease, a nickase, an integrase, reverse transcriptase, or a combination thereof.
318. The lipid particle, composition, method, or DNA sequence of embodiment 316 or embodiment 317, wherein the genome-modifying protein comprises a zinc-finger nuclease (ZFN), a transcription-activator like effector nucleases (TALEN), or a CRISPR-associated (Cas) protein.
319. The lipid particle, composition, method, or DNA sequence of any of embodiments 316-318, wherein the genome-modifying protein is a Cas protein.
320. The lipid particle, composition, method, or DNA sequence of any of embodiments 316-319, wherein the genome-modifying protein is (i) Cas9, optionally saCas9 or spCas9; or (ii) cpf1.
321. The lipid particle of any of embodiments 1-201 and 262, the composition of embodiment 263, the method of any of embodiments 202-261 and 264-267, or the DNA sequence of any of embodiments 268-281 and 312-315, wherein the heterologous protein is a tumor neoepitope.
22. The lipid particle of any of embodiments 1-201 and 262, the composition of embodiment 263, the method of any of embodiments 202-261 and 264-267, or the DNA sequence of any of embodiments 268-281 and 312-315, wherein the heterologous protein is a viral Spike(s) glycoprotein.
323. The lipid particle of any of embodiments 1-201 and 262, the composition of embodiment 263, the method of any of embodiments 202-261 and 264-267, or the DNA sequence of any of embodiments 268-281 and 312-315, wherein the heterologous protein is a protein from Zika virus, optionally Zika virus prM-E protein; tuberculosis; respiratory syncytial virus (RSV), optionally RSV fusion (RSV-F) protein; influenza virus, optionally influenza virus hemagglutinin (HA); rabies virus, optionally rabies virus glycoprotein (RABV-G); human cytolomegalovirus (CMV); hepatitis C virus; human immunodeficiency virus 1 (HIV-1), and Streptococcus.
324. The lipid particle of any of embodiments 1-201 and 262, the composition of embodiment 263, the method of any of embodiments 202-261 and 264-267, or the DNA sequence of any of embodiments 268-281 and 312-315, wherein the heterologous protein is an antibody or an antigen-binding fragment thereof.
325. A vector comprising the DNA sequence of any of embodiments 268-324.
326. A mammalian cell comprising the DNA sequence of any of embodiments 268-324 or the vector of embodiment 325.
327. The mammalian cell of embodiment 326, further comprising viral nucleic acid, wherein the viral nucleic acid lacks one or more genes involved in viral replication.
328. The mammalian cell of embodiment 327, wherein the viral nucleic acid comprises:
one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3);
a nucleic acid encoding a viral envelope protein; and/or
a nucleic acid encoding a viral packaging protein selected from one or more of gag, pol, rev and tat.
329. The mammalian cell of any of embodiments 326-328, further comprising a RNA sequence encoding a heterologous protein.
330. The mammalian cell of any of embodiments 326-329, further comprising a guide RNA (gRNA).
331. A transfer plasmid comprising a promoter operably linked to a RNA sequence encoding a gag protein or portion thereof comprising at least a gag start codon; a RNA sequence encoding a heterologous protein that is linked to the RNA sequence encoding a gag protein or portion thereof; and a 3′ long terminal repeat (3′ LTR).
332. A transfer plasmid comprising a promoter operably linked to a ribonucleic acid (RNA), wherein the RNA comprises, from 5′ to 3′: a 5′ long terminal repeat (5′ LTR); a gag 5′ untranslated region (UTR) or portion thereof comprising at least three nucleotides; a RNA sequence encoding a heterologous protein that is linked to the gag 5′ UTR or a portion thereof; and a 3′ long terminal repeat (3′ LTR).
333. A transfer plasmid comprising a promoter operably linked to a ribonucleic acid (RNA), wherein the RNA comprises, from 5′ to 3′: a 5′ long terminal repeat (5′ LTR); a retroviral packaging sequence; a gag start codon; a RNA sequence encoding a heterologous protein; and a 3′ long terminal repeat (3′ LTR).
334. The transfer plasmid of embodiment 333, wherein the retroviral packaging sequence comprises a mutation in a major splice donor site.
335. The transfer plasmid of embodiment 334, wherein the major splice donor site is a major splice donor site contained in SL2 of HIV psi.
336. The transfer plasmid of embodiment 334 or embodiment 335, wherein the mutation is a mutation that inhibits splicing at the major splice donor site.
337. The transfer plasmid of any one of embodiments 334-336, wherein the mutated major splice donor site comprises a mutation that prevents splicing at the major splice donor site.
338. A transfer plasmid comprising a promoter operably linked to a nucleic acid sequence encoding a fusion protein comprising a viral matrix (MA) protein and a MS2 coat protein (MS2_{cp}).
339. A transfer plasmid comprising a promoter operably linked to a nucleic acid sequence encoding a viral matrix (MA) protein and a heterologous protein.
340. The transfer plasmid of any of embodiments 331-337, wherein the transfer plasmid is a lentiviral transfer plasmid.

### EXAMPLES

The following examples are included for illustrative purposes only and are not intended to limit the scope of the invention.

### Example 1: Delivery of Genome Modifying Agents Using Genomic Viral mRNA

This Example describes the assessment of genetic engineering using vesicular stomatitis virus glycoprotein (VSV-G) pseudotyped lentivirus vectors (LVV) or virus-like particles (VLPs) to deliver genome modifying proteins using viral genomic mRNA. In these experiments, Cre was used as an exemplary genome modifying protein.

In a first experiment, transfer plasmid constructs were designed to produce LVV in which nucleic acid encoding Cre recombinase was packaged into the genomic mRNA of a VSV-G pseudotyped, reverse transcriptase (RT) deficient LVV. The transfer plasmids contained different constructs, as shown in Table 1. For example, constructs were engineered with and without a promoter, e.g. a cytomegalovirus (CMV) promoter, and in some cases, with an internal ribosome entry site (IRES) or a T2A self-cleaving peptide sequence. As a negative control, a construct not containing Cre mRNA was also provided (“construct 6”). A depiction of construct 5 is shown in FIG. 1.

TABLE 1
Genomic mRNA constructs

| Construct | | |
|---|---|---|
| No. | Name | SEQ ID NO |
| 1 | pMA2 CMV-Cre | 47 (nt) |
| 2 | pHAGE with Cre starting at the GAG | 48 (nt) |
| | start codon | |
| 3 | pMA2 CMV-EGFP-IRES-Cre | 49 (nt) |
| 4 | pMA2 IRES-Cre (no promoter) | 50 (nt) |
| 5 | pMA2 N-term GAG-T2A-Cre (no promoter) | 51 (nt); 72 (aa) |

| | |
|---|---|
| 6 | pMA2 CMV-GFP (negative control) |

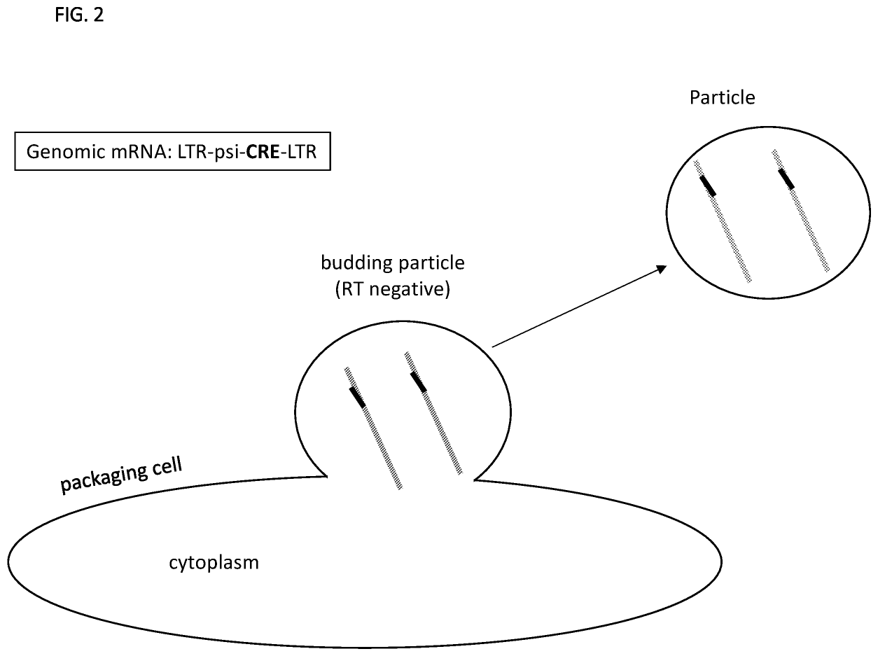

To achieve production of LVV containing the genomic lentiviral mRNA. 293T producer cells were transfected with a packaging reaction containing: (1) a transfer plasmid containing one of the constructs shown in Table 1. (2) a gag-pol packaging plasmid containing a D110E point mutation in pol to render the LVV reverse-transcriptase deficient. (3) a rev-containing packaging plasmid, (4) a tat-containing packaging plasmid, and (5) an envelope plasmid to achieve expression of the VSV-G envelope protein. Following LVV production, the cell culture was centrifuged to pellet the cells and the supernatant containing virus was collected. A schematic of the packaging process is depicted in FIG. 2.

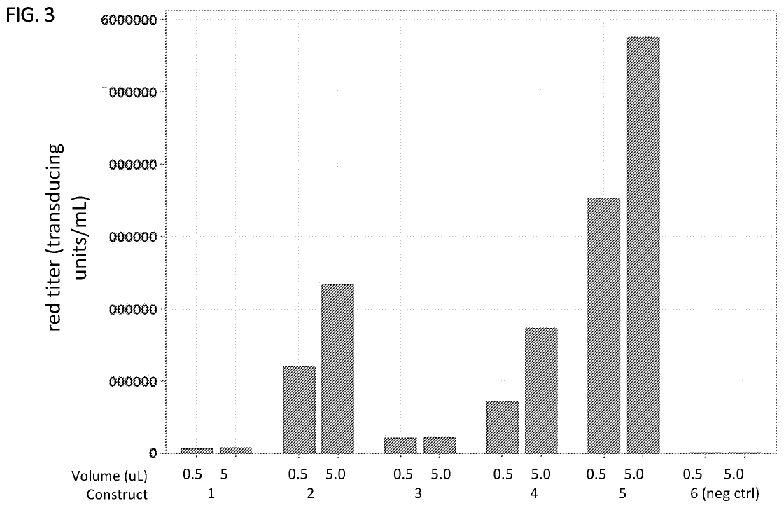

Two different volumes of supernatant containing LVV (0.5 μL or 5.0 μL) were titered onto HEK293 tdTomato reporter cells that were engineered with a lox-STOP-lox-tdTomato Cre reporter transgene, such that introduction of Cre into the cells excised the stop cassette to allow expression of the red fluorescent tdTomato protein in the cell. Expression of tdTomato was measured in transducing units per mL by calculating the fraction of tdTomato+ cells by flow cytometry, multiplied by the number of cells initially transduced, divided by the volume (mL) of virus used. LVV delivering constructs 2, 4, and 5 to the HEK reporter cells were observed to induce expression of tdTomato (FIG. 3).

In a related experiment, a mouse genetically engineered to express the lox-STOP-lox-tdTomato cre reporter transgene described above (Madisen et al., Nature Neurosci (2010) 13:133-40) was injected intravenously (IV) with 1.4×10⁸ transducing units (TU) of LVV delivering construct 5, and tdTomato expression was assessed. Approximately 2.6% and 2.1% of spleen and bone marrow cells, respectively, were found to express tdTomato, as assessed by flow cytometry.

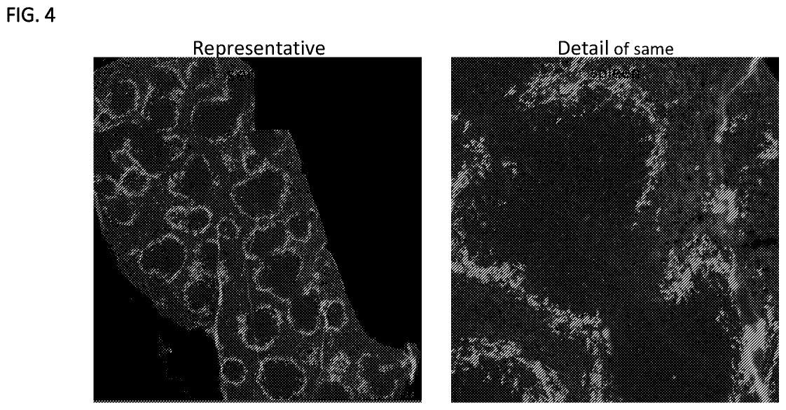

As an alternative delivery format, VLPs were generated by transfecting 293T producer cells with a packaging reaction containing the same five plasmids described above. The VLPs were injected IV into mice at 8.48×10⁶ vector genome (vg) per mouse, resulting in high expression of tdTomato in the spleen (FIG. 4; lighter shading indicates tdTomato expression).

Optionally, a Cas nuclease (e.g., Cas9) can be used as the genome-modifying protein instead of Cre. For example, a lentiviral vector is generated to encode a Cas9 nuclease. In some embodiments, a guide RNA (gRNA) engineered to contain MS2-binding loops (as in Example 2 below) is co-expressed during lentiviral vector production to enable CRISPR/Cas based gene editing by the resulting lipid particles containing Cas-encoding mRNA and a gRNA.

### Example 2: Delivery of Genome Modifying Agents by Tethering RNA to Viral Particles

This Example describes the assessment of genetic engineering using VSV-G pseudotyped LVV. VLPs, or gesicles to deliver genome modifying proteins by tethering mRNA to the interior of viral particles. In these experiments, Cre was used as an exemplary genome modifying protein.

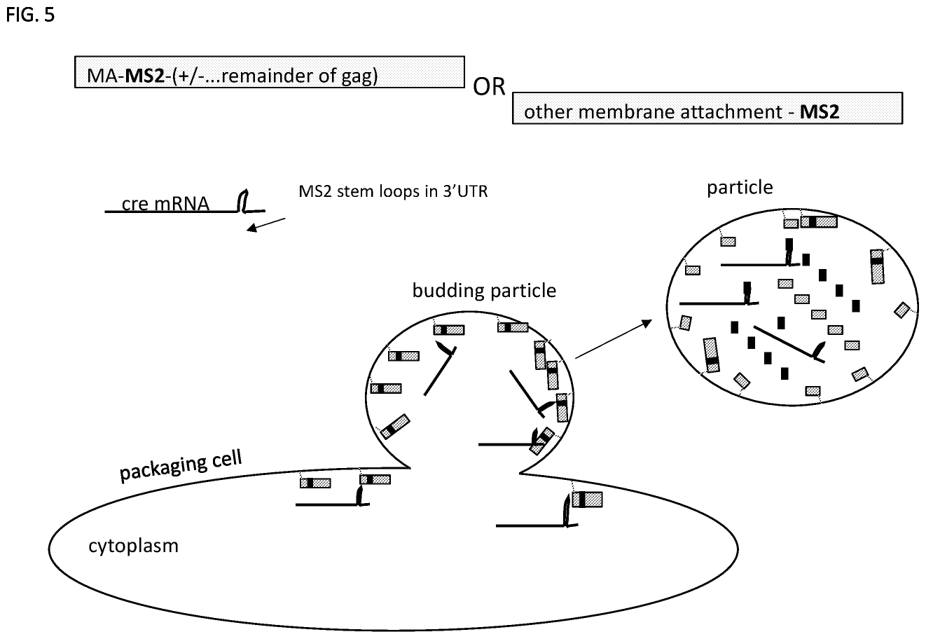

A MA-MS2_{cp}-CA gagpol construct (“construct 7”; Table 2) was used to tether co-expressed Cre-encoding mRNA bearing MS2 stem loops in the 3′ untranslated region (UTR) to the interior of VSV-G pseudotyped viral particles. Briefly, an expression plasmid was generated to provide Cre-encoding mRNA containing either 12 or 24 MS2 stem loop sequences capable of binding the MS2 coat protein (MS2_{cp}). A lentiviral transfer plasmid was designed to contain a sequence encoding for MS2_{cp} inserted into the gag-pol construct between the matrix (MA) and capsid (CA) sequences. Thus, Cre-encoding mRNA was able to bind to the gag-pol polyprotein expressed within viral and virus-like particles at the MS2_{cp} by virtue of its MS2 stem loops. A schematic of the packaging process is depicted in FIG. 5. As a negative control, Cre containing no MS2 stem loop sequences (0) was also provided.

LVV was produced by transfecting 293T cells with a packaging reaction containing: (1) a rev-containing packaging plasmid, (2) a tat-containing packaging plasmid, (3) the MA-MS2_{cp}-CA gag-pol transfer plasmid, (4) an envelope plasmid to drive expression of VSV-G, (5) the Cre expression plasmid, and (6) a CMV-EGFP transfer plasmid. VLPs were produced by transfecting 293T cells with a packaging reaction containing: (1) the MA-MS2_{cp}-CA gag-pol transfer plasmid, (2) an envelope plasmid to drive expression of VSV-G, and (3) the Cre expression plasmid.

TABLE 2
mRNA-tethering construct

| Construct | | |
|---|---|---|
| No. | Name | SEQ ID NO |
| 7 | MA-MS2_{cp}-CA gagpol | 53 (nt); 73 (aa) |

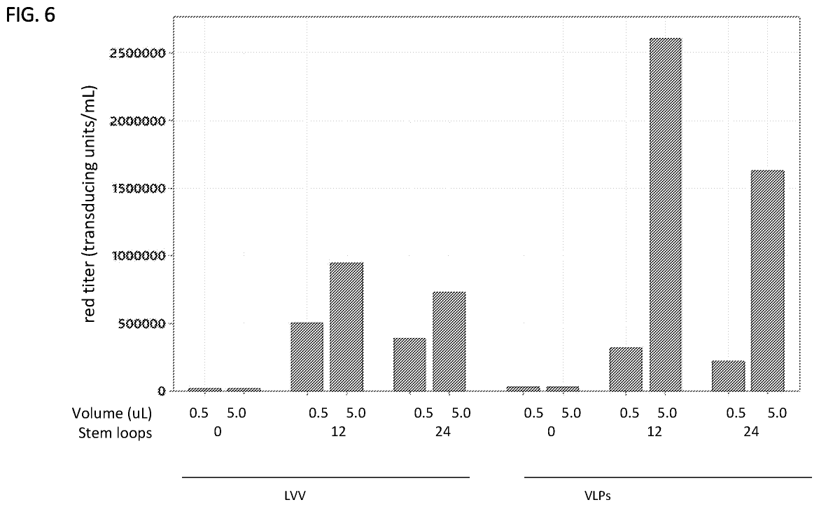

Viral supernatant (0.5 μL or 5.0 μL) was titered onto HEK293 tdTomato reporter cells and expression of tdTomato was measured as described in Example 1. Construct 7 was observed to induce expression of tdTomato in cells when provided in either LVV or VLP format (FIG. 6).

As an alternative format independent of gag-pol, Cre-encoding mRNA was tethered to MS2_{cp} in budding particles known as gesicles, which are produced by overexpression of VSV-G in cells (e.g., HEK cells). Gesicles were produced by transfecting cells with a packaging reaction containing: (1) an expression plasmid providing Cre-encoding mRNA, (2) a transfer plasmid encoding a fusion protein of a membrane attachment domain (MAD) attached to MS2_{cp}, and (3) an envelope plasmid to drive expression of the VSV-G envelope protein. In some cases, constructs were generated to incorporate MADs described in Aoki et al., Gene Therapy (2010) 17:1124-33. The exemplary generated constructs incorporated into the transfer plasmids are shown in Table 3.

TABLE 3
mRNA-tethering constructs

| Construct | | |
|---|---|---|
| No. | Name (MAD-MS2_{cp}) | SEQ ID NO |
| 8 | N-term PH domain-MS2_{cp} | 54 (nt) |
| 9 | C-term PH domain-MS2_{cp} | 55 (nt) |
| 10 | Myr (n-term; lyn pal⁻)-MS2_{cp} | 56 (nt) |
| 11 | Single Pal, from GNA12-MS2_{cp} | 57 (nt) |
| 12 | Double Pal, from GNA13-MS2_{cp} | 58 (nt) |
| 13 | Triple Pal from GNA15-MS2_{cp} | 59 (nt) |
| 14 | Myr-pal (lyn), N-term-MS2_{cp} | 60 (nt) |
| 15 | Farnesyl, c-term, from HRAS-MS2_{cp} | 61 (nt) |
| 16 | HIV MA (N-term)-MS2_{cp} | 62 (nt); 74 (aa) |

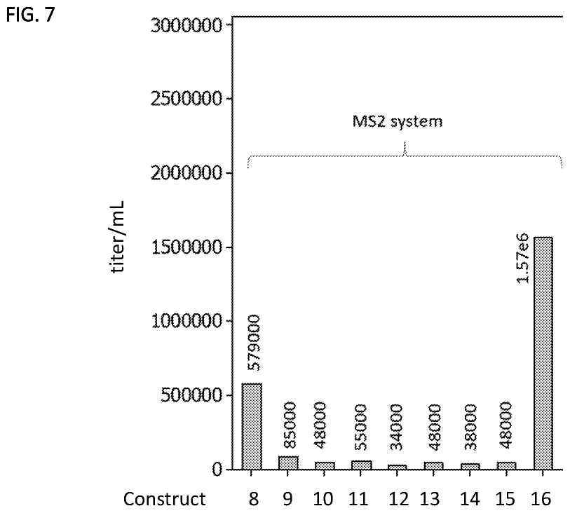

The viral titer of the different gesicles produced by each of the strategies was assessed (FIG. 7). Gesicles packaging Cre-encoding mRNA having 12 MS2-binding stem loops and delivering construct 16 were selected for further analysis, as this produced the highest viral titer.

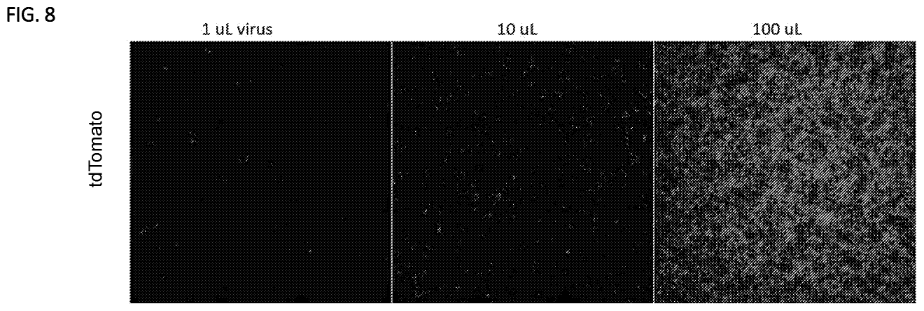

1 μL, 10 μL or 100 μL of supernatant containing the produced gesicles was added onto HEK293 tdTomato reporter cells that were engineered with a lox-STOP-lox-tdTomato Cre reporter transgene as described in Example 1. Incubation of HEK293 tdTomato reporter cells with 100 μL of the gesicles was observed to induce robust expression of tdTomato (FIG. 8; light shading indicates tdTomato expression). Mice expressing the tdTomato cre reporter transgene as described in Example 1 were injected intramuscularly (IM) in the gastrocnemius with 1.66×10⁶ vg of gesicles, which was observed to induce tdTomato expression throughout the muscle tissue (data not shown).

### Example 3: Delivery of Genome Modifying Agents by Tethering Proteins to Viral Particles

This Example describes the assessment of genetic engineering using VSV-G pseudotyped gesicles to deliver genome modifying proteins by tethering the proteins to the interior of viral particles. In these experiments, Cre was used as an exemplary genome modifying protein.

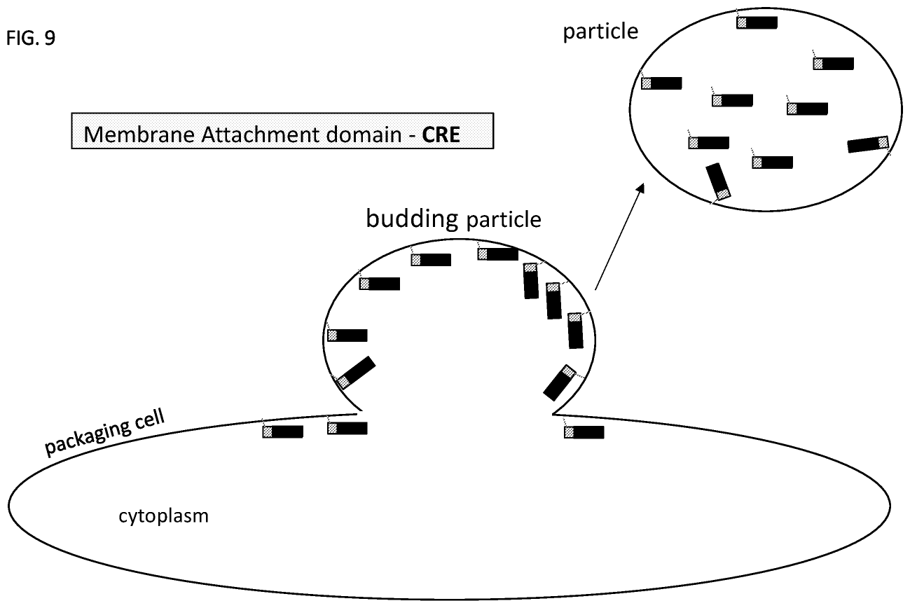

Reversible membrane binding domains (Table 4) were used to tether Cre recombinase to the interior of gesicles. Briefly, 293T cells were transfected with a packaging reaction containing: (1) a transfer plasmid encoding a membrane attachment domain (MAD) and Cre recombinase (expressed as a MAD-Cre fusion protein) and (2) an envelope plasmid to drive expression of VSV-G. A schematic of the method is depicted in FIG. 9.

TABLE 4
Protein-tethering constructs

| Construct | | |
|---|---|---|
| No. | Name (MAD-Cre) | SEQ ID NO |
| 17 | N-term PH domain-Cre | 63 (nt) |
| 18 | C-term PH domain-Cre | 64 (nt) |
| 19 | Myr (n-term; lyn pal⁻)-Cre | 65 (nt) |
| 20 | Single Pal, from GNA12-Cre | 66 (nt) |
| 21 | Double Pal, from GNA13-Cre | 67 (nt) |
| 22 | Triple Pal from GNA15-Cre | 68 (nt) |
| 23 | Myr-pal (lyn), N-term-Cre | 69 (nt) |
| 24 | Farnesyl, c-term, from HRAS-Cre | 70 (nt) |
| 25 | HIV MA (N-term)-Cre | 71 (nt); 75 (aa) |

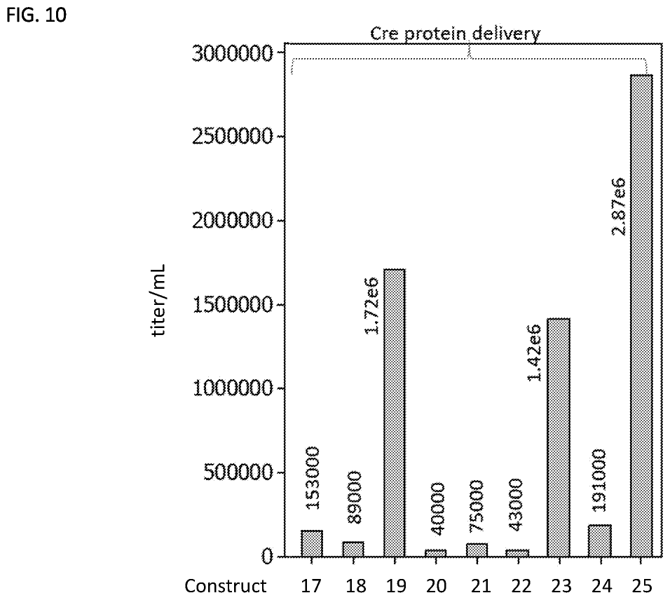

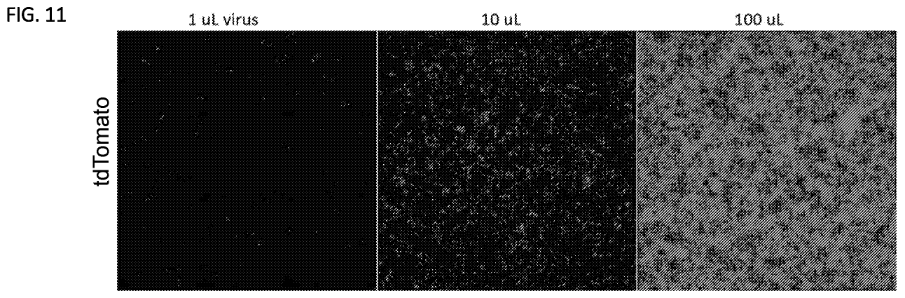

The exemplary generated constructs encoding the reversible binding domains are shown in Table 4. The viral titer of the different gesicles produced by using each of the above constructs was assessed (FIG. 10). Gesicles packaging construct 25 were optimized to provide a titer of 1.8×10⁷, and tested for further analysis, as these produced gesicles with the highest viral titer. Incubation of HEK293 tdTomato reporter cells with 100 μL of the gesicles was observed to induce robust expression of tdTomato (FIG. 11). Mice expressing the tdTomato Cre reporter transgene as described in Example 1 were injected intramuscularly (IM) in the gastrocnemius with 6.2×10⁷ vg of the gesicles packaging construct 25. tdTomato expression was observed throughout the gastrocnemius (data not shown).

### Example 4: In Vivo Comparison of Cre Delivery Formats

The ability of LVV, VLPs, and gesicles, described in Examples 1-3 above, to deliver Cre to the bone marrow, peripheral blood, and spleen of tdTomato Cre reporter mice was compared.

Constructs provided in different formats (i.e., gesicles, LVV, or VLP), each as described in the preceding Examples, were compared (Table 5).

TABLE 5
Constructs and formats compared for in vivo Cre delivery

| | | | Format |
|---|---|---|---|
| Construct | | | (amount of |
| No. | Name | SEQ ID NO | virus injected) |
| 16 | MS2_{cp}-Cre mRNA; | 62 (nt); 74 (aa) | gesicles (1.0 × 10⁷) |
| | HIV MA (N-term) | | |
| 25 | MA-Cre; | 71 (nt); 75 (aa) | gesicles (3.9 × 10⁸) |
| | HIV MA (N-term) | | |
| 1 | pMA2 CMV-Cre | 47 (nt) | LVV (3.9 × 10⁸) |
| 5 | pMA2 N-term GAG- | 51 (nt); 72 (aa) | VLP (5.3 × 10⁷) |
| | T2A-Cre | | |

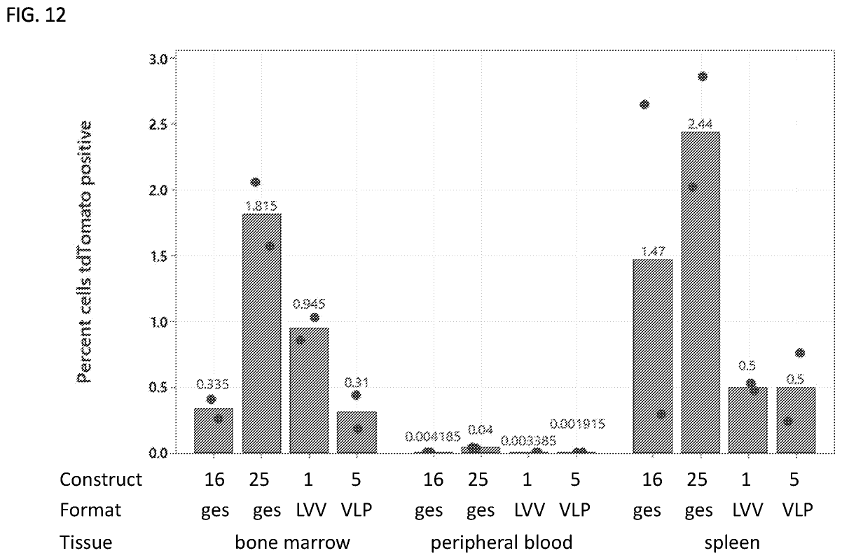

tdTomato cre reporter mice as described in previous examples were injected IV with gesicles (ges), lentiviral vector (LVV), or virus-like particles (VLP), as shown in Table 5. The amount of LVV or VLP injected was based on the functional titer previously determined using HEK293 tdTomato reporter cells. 10 days following injection, bone marrow (BM), peripheral blood (PB), and spleen were collected, and the percentage of tdTomato-expressing cells in each was determined by flow cytometry (FIG. 12).

The spleens of mice injected with gesicles packaging construct 25 at a titer of 6.2×10⁷ vg exhibited robust and widespread expression of tdTomato. Modest tdTomato expression was also observed in non-hepatocyte liver samples across all four groups of mice.

### Dosing and Long-Term In Vivo Studies

The ability of LVV, VLPs, and gesicles, described in Examples 1-3 above, to deliver Cre to the bone marrow, peripheral blood, and spleen of tdTomato Cre reporter mice was compared with different doses and over time.

Construces were intravenously injected into mice to compare the different formats. Costructs and their doses are provided below in Table 6.

TABLE 6
Constructs and formats compared for in vivo lon-term Cre delivery

| | | | Dose per mouse |
|---|---|---|---|
| Construct | | | (Transducing units |
| No. | Name | SEQ ID NO | on 293T cells) |
| 1 | pMA2 CMV-Cre | 47 (nt) | LVV (4.35 × 10⁹) |
| 5 | pMA2 N-term | 51 (nt); 72 (aa) | VLP (3.94 × 10⁸) |
| | GAG-T2A-Cre | | |
| 16 | MS2_{cp}-Cre mRNA; | 62 (nt); 74 (aa) | Gesicles (4.23 × 10⁷) |
| | HIV MA (N-term) | | |
| 25 dose 1 | MA-Cre; | 71 (nt); 75 (aa) | Gesicles (6.06 × 10⁸) |
| | HIV MA (N-term) | | |
| 25 dose 2 | MA-Cre; | 71 (nt); 75 (aa) | Gesicles (7.43 × 10⁸) |
| | HIV MA (N-term) | | |
| 25 dose 3 | MA-Cre; | 71 (nt); 75 (aa) | Gesicles (3.43 × 10⁸) |
| | HIV MA (N-term) | | |

After the mice were injected, 17 days post injection and 223 days post injection the mice were collected and analyzed for percent of cells that are tdTomato positive in the peripheral blood (PB), spleen, bone marrow (BM) and for the 223 days post injection timepoint hematopoetic stem cells (HSCs). The results for 17 days post injection can be seen in Table 7.

TABLE 7
Percent of cells tdTomato⁺ 17 days post injection

| Mouse | Construct | Name | PB | Spleen | BM |
|---|---|---|---|---|---|
| 1 | 1 | pMA2 CMV-Cre | 0.11 | 0.33 | 1.99 |
| 2 | 5 | pMA2 N-term | 0.07 | 2.15 | 12.00 |
| | | GAG-T2A-Cre | | | |
| 4 | 25 dose 1 | MA-Cre; | 1.21 | 2.30 | 21.00 |
| | | HIV MA (N-term) | | | |
| 5 | 25 dose 2 | MA-Cre; | 0.16 | 2.63 | 21.00 |
| | | HIV MA (N-term) | | | |
| 6 | 25 dose 3 | MA-Cre; | 0.15 | 2.54 | 24.00 |
| | | HIV MA (N-term) | | | |
| 19 | 16 | MS2_{cp}-Cre mRNA; | 0.12 | 1.57 | 3.90 |
| | | HIV MA (N-term) | | | |
| 26 | Control (N/A) | | 0.00 | 0.01 | 0.02 |

The results for 223 days post injection can be seen in Table 8.

TABLE 8
Percent of cells tdTomato⁺ 223 days post injection

| Mouse | Construct | Name | PB | Spleen | BM | HSCs |
|---|---|---|---|---|---|---|
| 20 | 1 | pMA2 CMV-Cre | 0.02 | 0.09 | 0.29 | 0.29 |
| 21 | 5 | pMA2 N-term | 0.01 | 1.64 | 0.69 | 0.60 |
| | | GAG-T2A-Cre | | | | |
| 22 | 25 dose 1 | MA-Cre; | 0.01 | 3.25 | 0.32 | 0.27 |
| | | HIV MA (N-term) | | | | |
| 23 | 25 dose 2 | MA-Cre; | 1.29 | 4.01 | 1.42 | 0.85 |
| | | HIV MA (N-term) | | | | |
| 24 | 25 dose 3 | MA-Cre; | 0.87 | 9.12 | 1.48 | 0.75 |
| | | HIV MA (N-term) | | | | |
| 25 | 16 | MS2_{cp}-Cre mRNA; | 0.10 | 4.45 | 1.10 | 0.80 |
| | | HIV MA (N-term) | | | | |
| 27 | Control | | 0.01 | 0.01 | 0.02 | 0.11 |
| | (N/A) | | | | | |

Cells across tissues expressed tdTomato following treatment with all the different formats (i.e., gesicles, LVV, or VLP). The earlier time point of 17 days post injection showed highest expression of tdTomato in the BM for all the formats. At the later timepoint, 223 days post injection, spleen showed the highest tdTomato expression for all of the formats except construct 1.

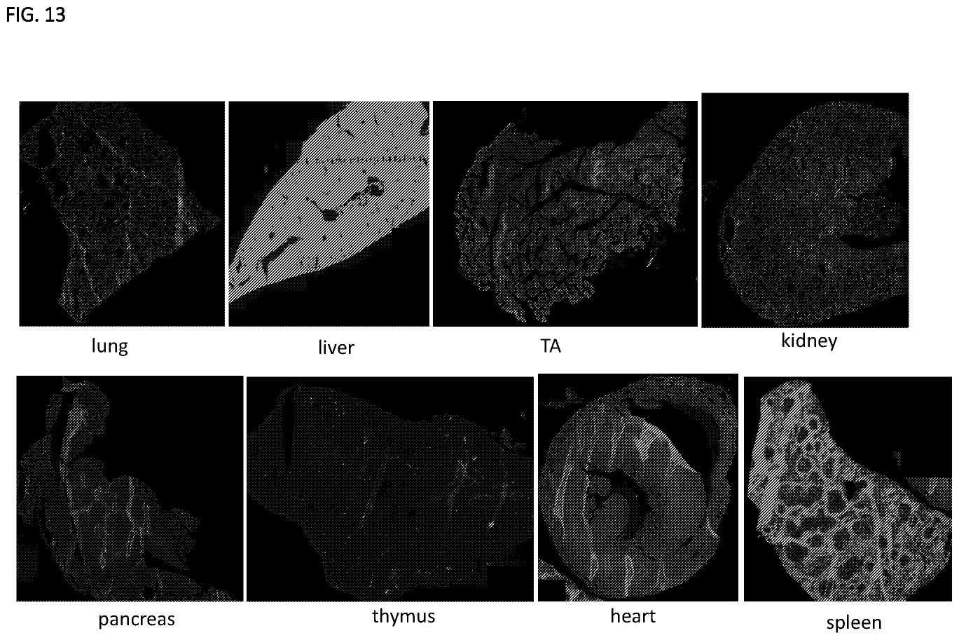

The diversity of tissue that the Cre delivery formats deliver to can be observed in FIG. 13, which displays tdTomato in different tissues after 17 days post injection with construct 25. Liver and spleen show complete transduction and all other tissues (lung, tibialis anterior (TA), kidney, pancreas, thyumus, and heart) have some level of transduction.

### Gesicles Show High Efficiency of Transducing Mouse Primary Activated T Cells

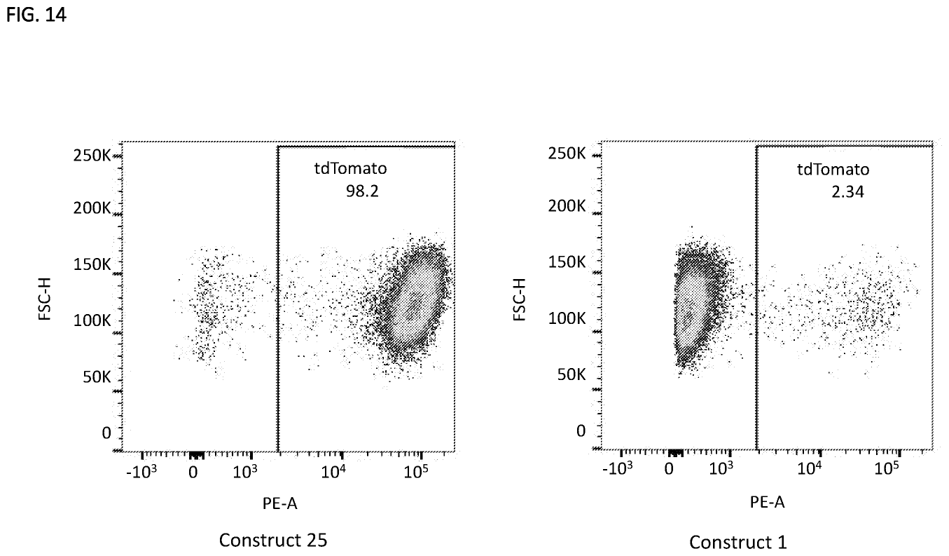

Mouse primary T cells were activated and transduced with Cre using the formats shown in Table 5. Construct 25 was used to carry out various multiplicity of infections (MOI) compared to lentiviral control (construct 1) on primary activated mouse T cells. Cre was delivered which results in the cells becoming positive for tdTomato as described earlier in this Example. The results are shown below in Table 9. The Ma-cre delivery shown with construct 25 showed high efficiency of transduction compared to construct 1. In addition, construct 1 showed a limit where increasing the MOI no longer increased the percent positive for tdTomato, which was low, compared to construct 25. This is shown in FIG. 14, which shows representative flow charts for the highest MOI of each construct.

TABLE 9
MOI comparison

| Construct | MOI | % Positive |
|---|---|---|
| 25 | 1825 | 98.2 |
| 25 | 365 | 91.6 |
| 25 | 73 | 72.5 |
| 25 | 14.6 | 42.2 |
| 25 | 2.92 | 12.3 |
| 25 | 0.584 | 1.54 |
| 1 | 3522.25 | 2.34 |
| 1 | 703 | 2.59 |
| 1 | 141 | 1.83 |
| 1 | 28.1 | 0.82 |
| 1 | 5.62 | 0.25 |
| 1 | 1.12 | 0.046 |

The titer of activated mouse T cells was measured for each of the different constructs which were positive for tdTomato and compared to the titer of 293T cells from previous examples that were positive for tdTomato red. The titer of tdTomato red 293T cells was divided by titer of activated mouse T cells to determine the fold decrease in titer for the in vivo studies. The results are shown in Table 10.

TABLE 10
Fold decrease in titer

| | Titer-activated | Titer-293T | Fold decrease |
|---|---|---|---|
| Construct | mouse T cells | tdTomato | in titer |
| 25 | 5.00 × 10⁸ | 7.32 × 10⁹ | 14.6 |
| 16 | 2.8 × 10⁵ | 4.10 × 10⁶ | 14.6 |
| 1 | 5.94 × 10⁶ | 1.41 × 10¹⁰ | 2373.7 |
| 5 | 1.64 × 10⁶ | 1.09 × 10⁸ | 66.6 |

The lentivirus (construct 1) showed the highest decrease in titer, demonstrating the three different formats of Cre delivery performed better in vivo.

### Example 5: Assessing Delivery Using Modified Constructs

Constructs were modified to deliver different heterologous proteins, use different fusogens, or mutated to assess delivery efficiencies. The new constructs are shown in Table 11a while constructs using viral genomes were expressed with the envelop protein shown in Table 11b. Exemplary heterologous proteins such as Cre with a nuclear localization signal (SEQ ID NO:201), plain Cre (SEQ ID NO: 202), or EGFP (SEQ ID NO: 203) were used.

TABLE 11a
Modified constructs

| | | | SEQ ID |
|---|---|---|---|
| | | SEQ ID with | without |
| Construct | | heterologous | heterologous |
| No. | Name | protein | protein |
| 26 | pMA2 N-term mutated | 141 (nt) | 204 (nt) |
| | MSD GAG-T2A-Cre | | |
| 27 | pMA2 N-term | 142 (nt) | 205 (nt) |
| | GAG-T2A-EGFP | | |
| 28 | pMA2 N-term mutated | 143 (nt) | 204 (nt) |
| | MSD GAG-T2A-EGFP | | |
| 29 | pMA2 N-term | 200 (nt) | 205 (nt) |
| | GAG-T2A-Cre | | |
| 30 | pMA2 N-term mutated | 141 (nt) | 204 (nt) |
| | MSD GAG-T2A-Cre | | |
| 31 | pMA2 N-term | 142 (nt) | 205 (nt) |
| | GAG-T2A-EGFP | | |
| 32 | pMA2 N-term mutated | 143 (nt) | 204 (nt) |
| | MSD GAG-T2A-EGFP | | |
| 33 | MS2_{cp}-EGFP mRNA; | 148 (nt) | 206 (nt) |
| | HIV MA (N-term) | | |
| 34 | MA-EGFP; | 149 (nt) | 207 (nt) |
| | HIV MA (N-term) | | |

TABLE 11b
Envelope protein expressed with each construct

| | | SEQ ID NO | SEQ ID NO |
|---|---|---|---|
| Construct | Envelope | with N-term | without N term |
| No. | protein | Methionine | Methionine |
| 26 | VSV-G | 189 | 199 |
| 27 | VSV-G | 189 | 199 |
| 28 | VSV-G | 189 | 199 |
| 29 | NiV F and | 144 (aa) and | 145 (aa) and |
| | NiV G | 146(aa) | 147(aa) |
| 30 | NiV F and | 144 (aa) and | 145 (aa) and |
| | NiV G | 146(aa) | 147(aa) |
| 31 | NiV F and | 144 (aa) and | 145 (aa) and |
| | NiV G | 146(aa) | 147(aa) |
| 32 | NiV F and | 144 (aa) and | 145 (aa) and |
| | NiV G | 146(aa) | 147(aa) |

### Mutated Major Splice Donor of HIV

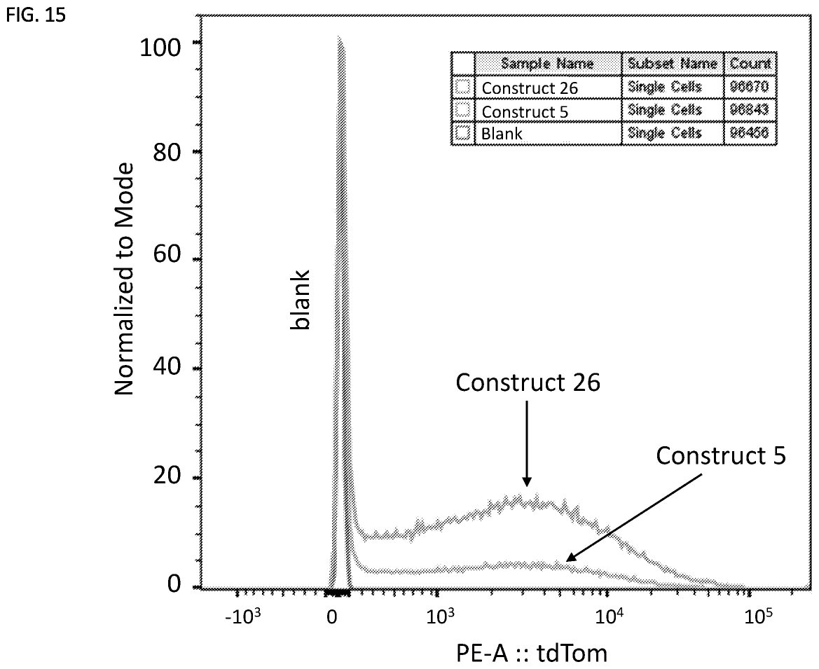

The major splice donor (MSD) in the psi region of HIV also for alternative splicing, which is important for HIV but unwanted in the present constructs as they result in non-functional sequences. The MSD was mutated so that all genomic vector transcripts are functional, full length, and packageable. of construct 5 from the above Examples was mutated to produce construct 26. Construct 26 was used to transduce cells with Cre to produce tdTomato positive cells as described in previous examples and compared to construct 5 to assess efficiency. As seen in FIG. 15, construct 26 shows an increased number of cells positive for tdTomato compared to construct 5, demonstrating increased transduction efficiency. This was performed with titers of VLPs of construct 5 having 2.4×10⁷/mL while construct 26 had 9.2×10⁷/mL.

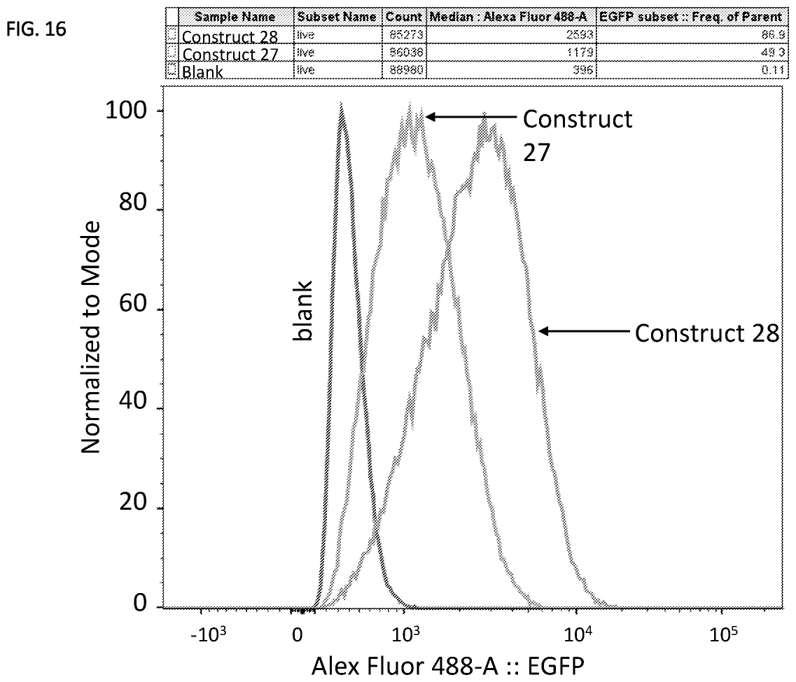

As previous Examples were done with Cre recombinase as the heterologous protein, construct 5 was further modified to no longer deliver Cre but instead deliver EGFP, herein referred to construct 27. The mutated MSD construct was also modified to deliver EGFP, herein referred to as construct 28. The constructs were used to transduce cells to assess the delivery of a different heterologous protein and to confirm the increased transduction using VLP with a mutated major splice donor. The results are shown in FIG. 16, which demonstrates construct 28 had increased cells positive for EGFP compared to construct 27. Both constructs were able to deliver EGFP to target cells This was performed with titers of 1.0×10⁷/mL for construct 27 while construct 28 had a titer of 4.7×10⁷/mL.

### Assessment of Alternative Fusogen

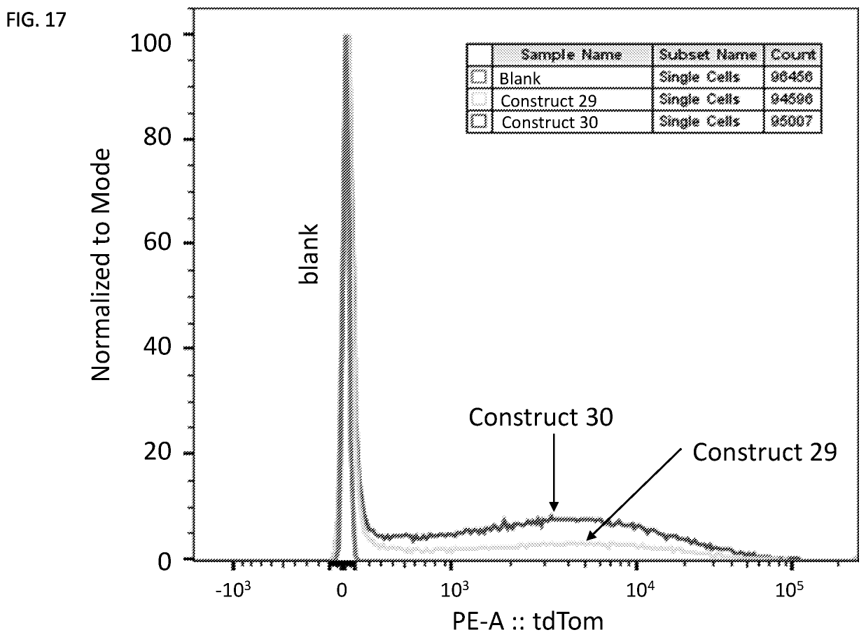

Previous constructs of VLP utilized VSV-G as the viral envelope protein to be used as a fusogen for the VLP particle to transduce a target cell. Construct 5 and construct 26 construct were modified to use NiV-F (SEQ ID NO:145) and NiV-G (SEQ ID NO: 147) as the fusogen rather than VSV-G, generating constructs 29 and 30 respectively. Cells were transduced with each of the constructs and the number of cells positive for tdTomato assessed. The results are shown in FIG. 17. Both constructs showed transduction demonstrating they work with an alternative fusogen, with construct 30 showing increased transduction compared to construct 29, demonstrating the mutated MSD increases transduction with an alternative fusogen. This was performed with a VLP titer for construct 29 of 2.3×10⁷/mL and a VLP titer for construct 30 of 5.0×10⁷/mL

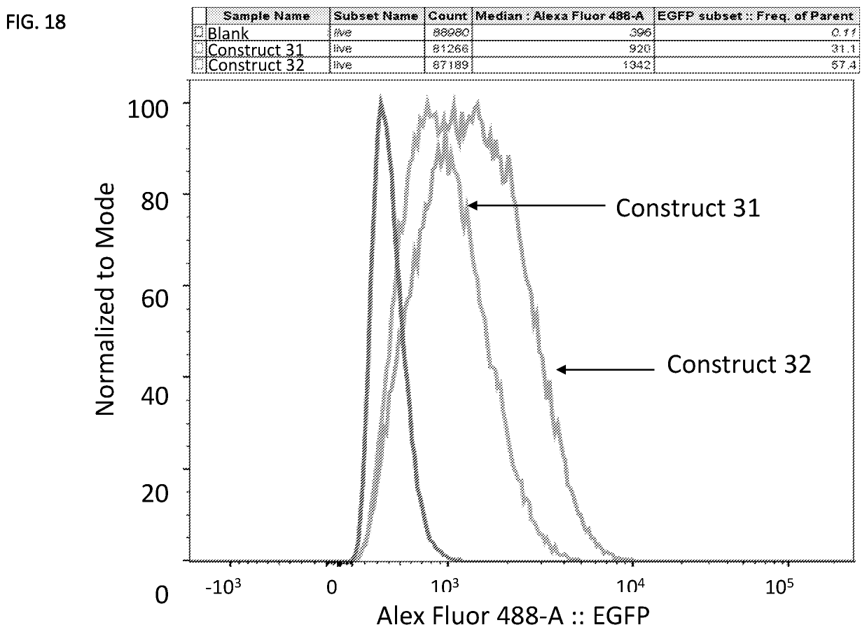

These two constructs were further modified to deliver EGFP to confirm the different fusogen works with an alternative heterologous protein. Construct 29 was modified to construct 31 to deliver EGFP and construct 30 was modified to construct 32 to deliver EGFP. Cells were transduced with each of constructs 31 and 32 and the number of cells positive for EGFP was assessed and are shown in FIG. 18. Both constructs were able to transduce cells, with NiV-F-mutated MSD-EGFP demonstrating an increase in EGFP. This was performed with titers of 1.7×10⁷ for NiV-F-mutated MSD-EGFP 1.7×10⁷ while NiV-F-gag-T2A-EGFP had a titer of 7.2×10⁶.

### Assessment of Delivery of EGFP Using Gesicles

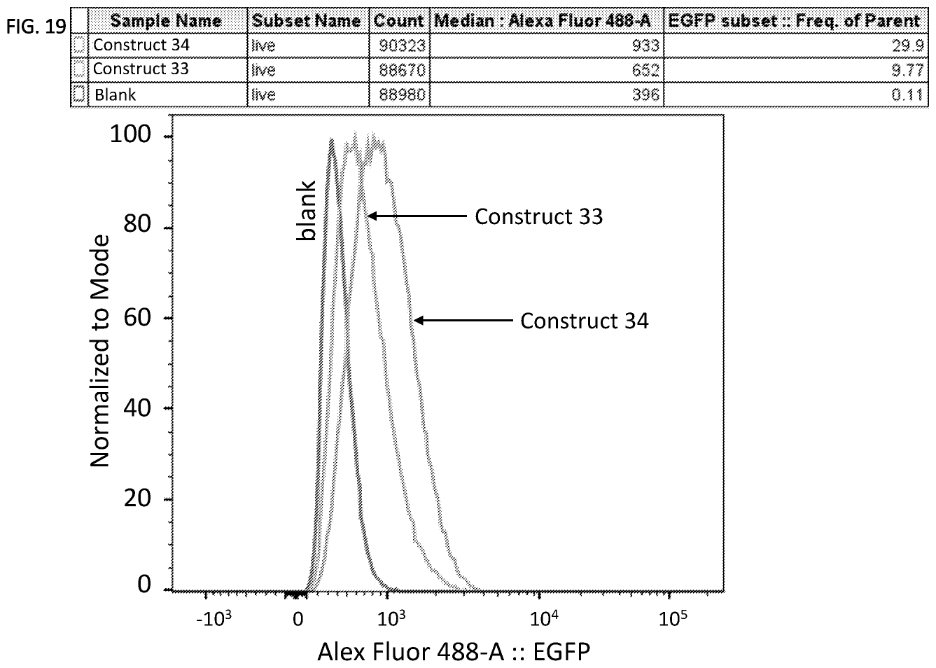

Previous experiments assessing gesicle delivery was done with deliver of Cre to tdTomato cells. To assess the use of gesicles to deliver a different heterologous protein, the constructs were modified to deliver EGFP. Construct 16 and construct 25 were modified to construct 33 and construct 34 respectively. Cells were transduced with both constructs and the results are shown in FIG. 19. Both constructs were able to transduce cells with EGFP, with construct 34 showing higher transduction. Titers of gesicles used to perform this experiment were 2.6×10⁶/mL for construct 34, while 3.1×10⁶/mL were used for construct 33.

### Example 6 Modifications for Tethering mRNA to the Interior of Viral Particles

Different configurations for tethering mRNA to the interior of viral particles were assessed by the use of different binders. The transductions were carried out similar to what is presented in Example 2, while varying the configuration of the constructs. Different numbers of MS2-binding stem loops, fusion of the RNA binding domain (i.e. MS2 coat protein (MS2_{cp})) domain onto the c-terminus of VSV-G, and use of phage lambda N protein (λN) as an RNA tethering protein were assessed.

For the use of phage lambda N protein (λN) as an RNA tethering protein, the MS2 coat protein (MS2_{cp}) as the RNA binding protein was replaced with the phage lambda N protein (λN). Varying numbers of boxB binding sites in the 3′UTR of the Cre-encoding mRNA was used to bind the cre-encoding mRNA to the interior of the pseudotyped viral particles instead of MS2-binding stem loops. In addition, a mutated phage lambda N protein (λN) which was previously described (Austin et al., J. Am. Chem. SOC. (2002) 124:10966-10967, hereby incorporated by reference in its entirety) was also assessed for transduction. The different binding configurations of fusion proteins are shown below in Table 12a.

TABLE 12a
Modified binders for tethering mRNA
to the interior of viral particles

| | | SEQ ID NO: first aa |
|---|---|---|
| | | sequence is with |
| Construct | | Methionine, second aa |
| No. | Name | sequence is without |
| 35 | MA-MS2_{cp} | 62 (nt); 134 (aa) 74 (aa) |
| 36 | MA-td-MS2_{cp} | 150 (nt); 190 (aa); 191 (aa) |
| 37 | VSVG-MS2_{cp} | 151 (nt); 157(aa); 192 (aa) |
| 38 | VSVG-td-MS2_{cp} | 152 (nt); 158 (aa); 193 (aa) |
| 39 | MA- λN | 153 (nt); 159 (aa); 194 (aa) |
| 40 | MA- mutλN | 154 (nt); 160 (aa): 195 (aa) |
| 41 | VSVG- λN | 155 (nt); 161 (aa); 196 (aa) |
| 42 | VSVG- mutλN | 156 (nt); 162 (aa); 197 (aa) |

TABLE 12b
RNA being tethered

| | | SEQ ID of plasmid |
|---|---|---|
| | | with stem loops with |
| Number of | SEQ ID of | Cre as exemplary |
| stem loops | stem loops | payload |
| 1x MS2 | 174 | 163 |
| 2x MS2 | 175 | 164 |
| 6x MS2 | 176 | 165 |
| 12x MS2 | 177 | 166 |
| 24x MS2 | 178 | 167 |
| 1x boxB | 179 | 168 |
| 2x boxB | 180 | 169 |
| 5x boxB | 181 | 170 |
| 10x boxB | 182 | 171 |
| 15x boxB | 183 | 172 |
| 20x boxB | 184 | 173 |

Gesicles were formed using the method as described in Example 2. Briefly, Cre-encoding mRNA was tethered to MS2_{cp} or lambda N protein (wild type or mutant) in budding particles known as gesicles, which are produced by overexpression of VSV-G (SEQ ID NO: 189) in cells (e.g., HEK cells). Gesicles were produced by transfecting cells with a packaging reaction containing: (1) an expression plasmid providing Cre-encoding mRNA (different configurations shown in Table 12b), (2) a transfer plasmid encoding a fusion protein of: (i) a viral matrix (MA) protein and MS2_{cp} (MA-MS2) (construct 35), or a MA protein and a tandem dimer of MS2_{cp} (MA-tdMS2) (construct 36); (ii) a VSV-G protein and MS2_{cp} (VSV-MS2) (construct 37), or a VSV-G protein and a tandem dimer of MS2_{cp} (VSV-tdMS2) (construct 38); (iii) a MA protein and a lambda N protein (MA-LN) (construct 39), or a MA protein and a mutant lambda N protein (MA-LN*) (construct 40); or (iv) a VSV-G protein and a lambda N protein (VSV-LN) (construct 41), or a VSV-G protein and a mutant lambda N protein (VSV-LN*) (construct 42), all of which are shown in Table 12a, and in some cases (3) an envelope plasmid to drive expression of a VSV-G envelope protein. In some cases, constructs were generated to incorporate MADs described in Aoki et al., Gene Therapy (2010) 17:1124-33. Expression plasmid sequences in Table 12b are shown with and without cre. Cre is an exemplary heterologous protein but others could be paired to the different stem loop configurations described. SEQ ID NO: 185 shows an exemplary MS2_{cp} stem loop encoded in DNA while an exemplary RNA sequence for the MS2_{cp} stem loop is set forth in SEQ ID NO: 208. SEQ ID NO: 186 shows an exemplary boxB stem loop. An exemplary tandem repeated MS2_{cp} domain such as used in construct 36 is set forth in SEQ ID NO: 198.

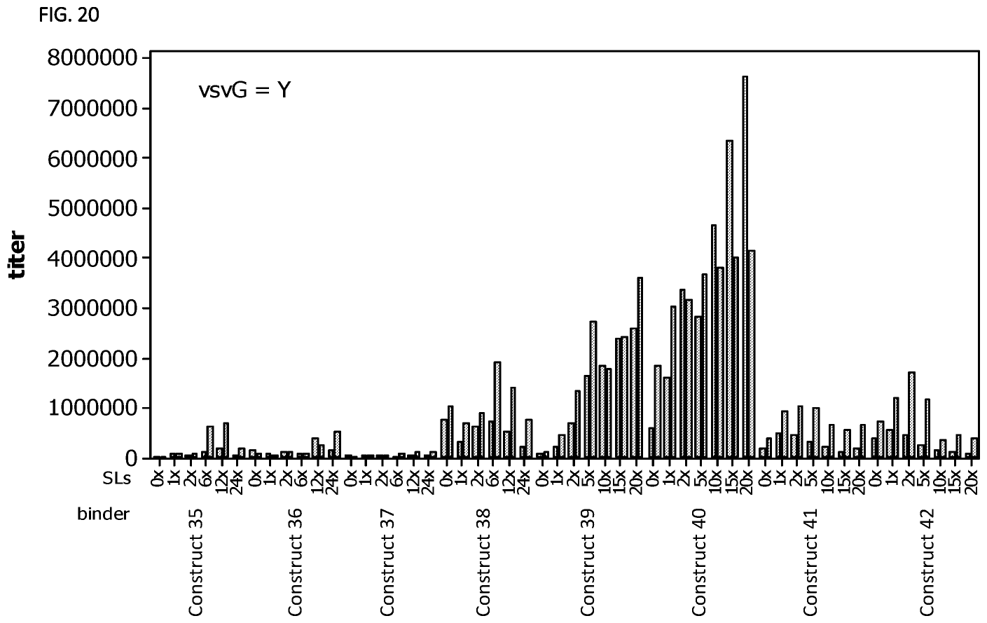

The titers of gesicles formed with each of the different constructs is shown in FIG. 20. The differing number of stem loops (SLs) as binding sites are shown for each construct along the x-axis. Construct 36 was the same as construct 35 but but with a tandem dimer MS2_{cp}. Construct 37 was the fusion of the MS2_{cp} domain onto the c-terminous of VSV-G. Construct 37 was the combination of a tandem dimer of MS2_{cp}, with the MS2_{cp} dimer domain fused onto the c-terminus of VSV-G. Construct 39 was the use of phage lambda N protein by the use of lentiviral transfer plasmid containing a sequence encoding for phage lambda N protein (SEQ ID NO: 187) inserted into the gag-pol construct between the matrix (MA) and capsid (CA) sequences. Construct 40 is the same construct as construct 39 but the previously described mutated phage lambda N protein was used (SEQ ID NO: 188). The domain with the lambda N protein was fused to the VSV-G coat to make mRNA-carrying gesicles with a single protein and are referred to as construct 41 for the wild type lambda N protein or construct 42 if the fusion was done with the mutated lambda N protein. As shown in FIG. 20, the use of lambda N protein for binding to boxB binding sites in the Cre-encoding mRNA lead to high titers. In addition, fusing the tandem repeats of MS2_{cp} to the VSV-G coat (VSV-tdMS2) showed increased gesicle titer formation.

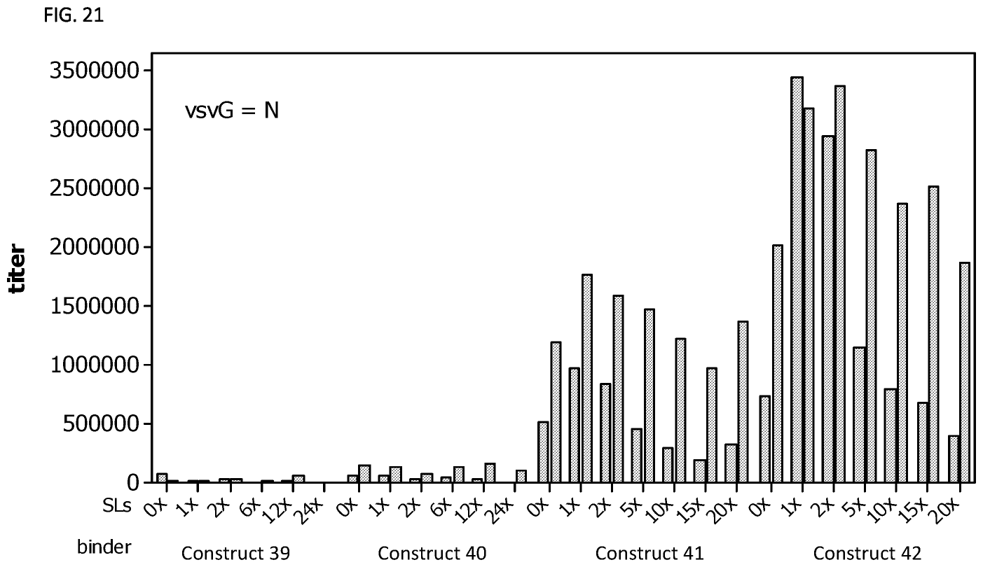

Since the fusion of either MS2_{cp} domain or lambda N protein to the VSV-G envelope protein should reduce the need for a separate plasmid to drive VSV-G envelope protein, the above experiment was repeated for the constructs 39-42 with either MS2_{cp} domain or lambda N protein fused to the VSV-G protein but the packaging reaction no longer contained item (3) an envelope plasmid to drive expression of the VSV-G envelope protein. The results are shown in FIG. 21. The constructs with lambda N protein (either wild type or mutated) fused with VSV-G were capable of producing high titers of gesicles, showing a reduced need for a separate plasmid for gesicle formation.

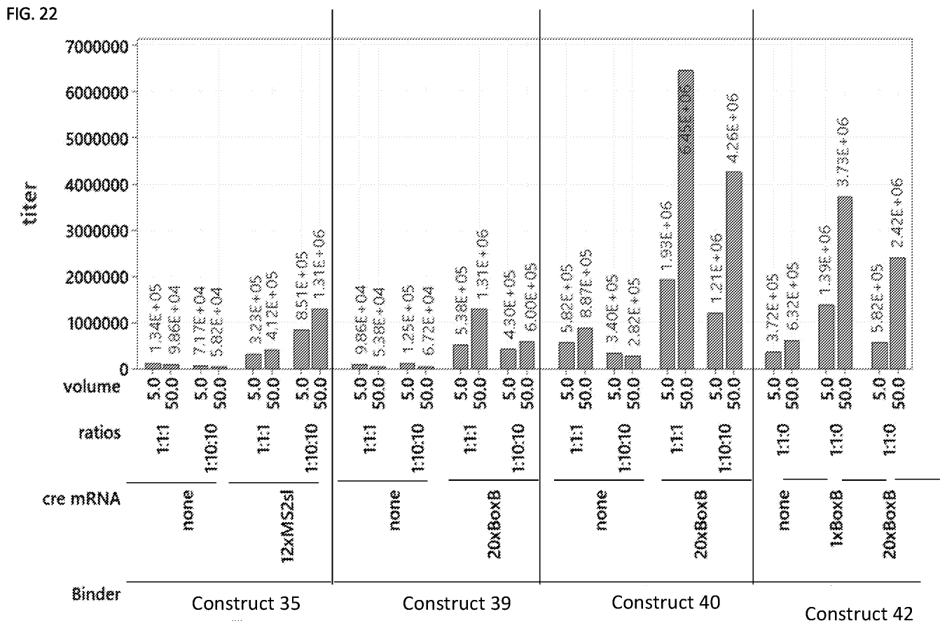

The ratios of the 3 plasmids (RNA binding protein, cre mRNA, and VSV-G envelop plasmids) used to form gesicles with the different constructs was optimized. The results are shown in FIG. 22. The binders used are similar to those described above, either construct 35, construct 39, construct 40, or construct 42. For construct 42 no VSV-G envelop plasmid was added to the packaging reaction since the mutated lambda N binding protein was fused to the c-terminus of VSV-G on the binder plasmid. The three numbers in the ratios in FIG. 22 refer to ratios of the following plasmids, RNA binder:cre mRNA:VSV-G protein to one another respectively. Different volumes of viral supernatant was applied to the reporter cells for titering and were also assessed to find the optimum titers for each construct.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | Description |
|---|---|---|
| 1 | MGPAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN ISCPNPLPFR | (602 aa) |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 2 | MMADSKLVSLNNNLSGKIKDQGKVIKNYYGTMDIKKINDGLLDSKIL | Hendra Virus |
| | GAFNTVIALLGSIIIIVMNIMIIQNYTRTTDNQALIKESLQSVQQQIKALT | G Protein |
| | DKIGTEIGPKVSLIDTSSTITIPANIGLLGSKISQSTSSINENVNDKCKFTL | |
| | PPLKIHECNISCPNPLPFREYRPISQGVSDLVGLPNQICLQKTTSTILKPR | |
| | LISYTLPINTREGVCITDPLLAVDNGFFAYSHLEKIGSCTRGIAKQRIIG | |
| | VGEVLDRGDKVPSMFMTNVWTPPNPSTIHHCSSTYHEDFYYTLCAVS | |
| | HVGDPILNSTSWTESLSLIRLAVRPKSDSGDYNQKYIAITKVERGKYD | |
| | KVMPYGPSGIKQGDTLYFPAVGFLPRTEFQYNDSNCPIIHCKYSKAEN | |
| | CRLSMGVNSKSHYILRSGLLKYNLSLGGDIILQFIEIADNRLTIGSPSKI | |
| | YNSLGQPVFYQASYSWDTMIKLGDVDTVDPLRVQWRNNSVISRPGQS | |
| | QCPRFNVCPEVCWEGTYNDAFLIDRLNWVSAGVYLNSNQTAENPVF | |
| | AVFKDNEILYQVPLAEDDTNAQKTITDCFLLENVIWCISLVEIYDTGDS | |
| | VIRPKLFAVKIPAQCSES | |
| 3 | MADSKLVSLNNNLSGKIKDQGKVIKNYYGTMDIKKINDGLLDSKILG | Hendra Virus |
| | AFNTVIALLGSIIIIVMNIMIIQNYTRTTDNQALIKESLQSVQQQIKALTD | G Protein |
| | KIGTEIGPKVSLIDTSSTITIPANIGLLGSKISQSTSSINENVNDKCKFTLP | without Met |
| | PLKIHECNISCPNPLPFREYRPISQGVSDLVGLPNQICLQKTTSTILKPRL | |
| | ISYTLPINTREGVCITDPLLAVDNGFFAYSHLEKIGSCTRGIAKQRIIGV | |
| | GEVLDRGDKVPSMFMTNVWTPPNPSTIHHCSSTYHEDFYYTLCAVSH | |
| | VGDPILNSTSWTESLSLIRLAVRPKSDSGDYNQKYIAITKVERGKYDK | |
| | VMPYGPSGIKQGDTLYFPAVGFLPRTEFQYNDSNCPIIHCKYSKAENC | |
| | RLSMGVNSKSHYILRSGLLKYNLSLGGDIILQFIEIADNRLTIGSPSKIY | |
| | NSLGQPVFYQASYSWDTMIKLGDVDTVDPLRVQWRNNSVISRPGQSQ | |
| | CPRFNVCPEVCWEGTYNDAFLIDRLNWVSAGVYLNSNQTAENPVFA | |
| | VFKDNEILYQVPLAEDDTNAQKTITDCFLLENVIWCISLVEIYDTGDSV | |
| | IRPKLFAVKIPAQCSES | |
| 4 | MPAENKKVRFENTTSDKGKIPSKVIKSYYGTMDIKKINEGLLDSKILSA | Nipah Virus G |
| | FNTVIALLGSIVIIVMNIMIIQNYTRSTDNQAVIKDALQGIQQQIKGLAD | Protein |
| | KIGTEIGPKVSLIDTSSTITIPANIGLLGSKISQSTASINENVNEKCKFTLP | |
| | PLKIHECNISCPNPLPFREYRPQTEGVSNLVGLPNNICLQKTSNQILKPK | |
| | LISYTLPVVGQSGTCITDPLLAMDEGYFAYSHLERIGSCSRGVSKQRII | |
| | GVGEVLDRGDEVPSLFMTNVWTPPNPNTVYHCSAVYNNEFYYVLCA | |
| | VSTVGDPILNSTYWSGSLMMTRLAVKPKSNGGGYNQHQLALRSIEKG | |
| | RYDKVMPYGPSGIKQGDTLYFPAVGFLVRTEFKYNDSNCPITKCQYS | |
| | KPENCRLSMGIRPNSHYILRSGLLKYNLSDGENPKVVFIEISDQRLSIGS | |
| | PSKIYDSLGQPVFYQASFSWDTMIKFGDVLTVNPLVVNWRNNTVISRP | |
| | GQSQCPRFNTCPEICWEGVYNDAFLIDRINWISAGVFLDSNQTAENPV | |
| | FTVFKDNEILYRAQLASEDTNAQKTITNCFLLKNKIWCISLVEIYDTGD | |
| | NVIRPKLFAVKIPEQCT | |
| 5 | PAENKKVRFENTTSDKGKIPSKVIKSYYGTMDIKKINEGLLDSKILSAF | Nipah Virus G |
| | NTVIALLGSIVIIVMNIMIIQNYTRSTDNQAVIKDALQGIQQQIKGLADK | Protein (No |
| | IGTEIGPKVSLIDTSSTITIPANIGLLGSKISQSTASINENVNEKCKFTLPP | Met) |
| | LKIHECNISCPNPLPFREYRPQTEGVSNLVGLPNNICLQKTSNQILKPKL | |
| | ISYTLPVVGQSGTCITDPLLAMDEGYFAYSHLERIGSCSRGVSKQRIIG | |
| | VGEVLDRGDEVPSLFMTNVWTPPNPNTVYHCSAVYNNEFYYVLCAV | |
| | STVGDPILNSTYWSGSLMMTRLAVKPKSNGGGYNQHQLALRSIEKGR | |
| | YDKVMPYGPSGIKQGDTLYFPAVGFLVRTEFKYNDSNCPITKCQYSKP | |
| | ENCRLSMGIRPNSHYILRSGLLKYNLSDGENPKVVFIEISDQRLSIGSPS | |
| | KIYDSLGQPVFYQASFSWDTMIKFGDVLTVNPLVVNWRNNTVISRPG | |
| | QSQCPRFNTCPEICWEGVYNDAFLIDRINWISAGVFLDSNQTAENPVF | |
| | TVFKDNEILYRAQLASEDTNAQKTITNCFLLKNKIWCISLVEIYDTGDN | |
| | VIRPKLFAVKIPEQCT | |
| 6 | MLSQLQKNYLDNSNQQGDKMNNPDKKLSVNFNPLELDKGQKDLNK | Cedar Virus G |
| | SYYVKNKNYNVSNLLNESLHDIKFCIYCIFSLLIIITIINIITISIVITRLKV | Protein |
| | HEENNGMESPNLQSIQDSLSSLTNMINTEITPRIGILVTATSVTLSSSINY | |
| | VGTKTNQLVNELKDYITKSCGFKVPELKLHECNISCADPKISKSAMYS | |
| | TNAYAELAGPPKIFCKSVSKDPDFRLKQIDYVIPVQQDRSICMNNPLL | |
| | DISDGFFTYIHYEGINSCKKSDSFKVLLSHGEIVDRGDYRPSLYLLSSH | |
| | YHPYSMQVINCVPVTCNQSSFVFCHISNNTKTLDNSDYSSDEYYITYF | |
| | NGIDRPKTKKIPINNMTADNRYIHFTFSGGGGVCLGEEFIIPVTTVINTD | |
| | VFTHDYCESFNCSVQTGKSLKEICSESLRSPINSSRYNLNGIMIISQNN | |
| | MTDFKIQLNGITYNKLSFGSPGRLSKTLGQVLYYQSSMSWDTYLKAG | |
| | FVEKWKPFTPNWMNNTVISRPNQGNCPRYHKCPEICYGGTYNDIAPL | |
| | DLGKDMYVSVILDSDQLAENPEITVFNSTTILYKERVSKDELNTRSTTT | |
| | SCFLFLDEPWCISVLETNRFNGKSIRPEIYSYKIPKYC | |
| 7 | LSQLQKNYLDNSNQQGDKMNNPDKKLSVNFNPLELDKGQKDLNKSY | Cedar Virus G |
| | YVKNKNYNVSNLLNESLHDIKFCIYCIFSLLIIITIINIITISIVITRLKVHEE | Protein (No |
| | NNGMESPNLQSIQDSLSSLTNMINTEITPRIGILVTATSVTLSSSINYVGT | Met) |
| | KTNQLVNELKDYITKSCGFKVPELKLHECNISCADPKISKSAMYSTNA | |
| | YAELAGPPKIFCKSVSKDPDFRLKQIDYVIPVQQDRSICMNNPLLDISD | |
| | GFFTYIHYEGINSCKKSDSFKVLLSHGEIVDRGDYRPSLYLLSSHYHPY | |
| | SMQVINCVPVTCNQSSFVFCHISNNTKTLDNSDYSSDEYYITYFNGIDR | |
| | PKTKKIPINNMTADNRYIHFTFSGGGGVCLGEEFIIPVTTVINTDVFTHD | |
| | YCESFNCSVQTGKSLKEICSESLRSPTNSSRYNLNGIMIISQNNMTDFKI | |
| | QLNGITYNKLSFGSPGRLSKTLGQVLYYQSSMSWDTYLKAGFVEKW | |
| | KPFTPNWMNNTVISRPNQGNCPRYHKCPEICYGGTYNDIAPLDLGKD | |
| | MYVSVILDSDQLAENPEITVFNSTTILYKERVSKDELNTRSTTTSCFLFL | |
| | DEPWCISVLETNRFNGKSIRPEIYSYKIPKYC | |
| 8 | MPQKTVEFINMNSPLERGVSTLSDKKTLNQSKITKQGYFGLGSHSERN | Bat |
| | WKKQKNQNDHYMTVSTMILEILVVLGIMENLIVLTMVYYQNDNINQ | Paramyxovirus |
| | RMAELTSNITVLNLNLNQLTNKIQREIIPRITLIDTATTITIPSAITYILAT | G Protein |
| | LTTRISELLPSINQKCEFKTPTLVLNDCRINCTPPLNPSDGVKMSSLATN | |
| | LVAHGPSPCRNFSSVPTIYYYRIPGLYNRTALDERCILNPRLTISSTKFA | |
| | YVHSEYDKNCTRGFKYYELMTFGEILEGPEKEPRMFSRSFYSPTNAVN | |
| | YHSCTPIVTVNEGYFLCLECTSSDPLYKANLSNSTFHLVILRHNKDEKI | |
| | VSMPSFNLSTDQEYVQIIPAEGGGTAESGNLYFPCIGRLLHKRVTHPLC | |
| | KKSNCSRTDDESCLKSYYNQGSPQHQVVNCLIRIRNAQRDNPTWDVI | |
| | TVDLTNTYPGSRSRIFGSFSKPMLYQSSVSWHTLLQVAEITDLDKYQL | |
| | DWLDTPYISRPGGSECPFGNYCPTVCWEGTYNDVYSLTPNNDLFVTV | |
| | YLKSEQVAENPYFAIFSRDQILKEFPLDAWISSARTTTISCFMFNNEIW | |
| | CIAALEITRLNDDIIRPIYYSFWLPTDCRTPYPHTGKMTRVPLRSTYNY | |
| 9 | PQKTVEFINMNSPLERGVSTLSDKKTLNQSKITKQGYFGLGSHSERNW | Bat |
| | KKQKNQNDHYMTVSTMILEILVVLGIMFNLIVLTMVYYQNDNINQR | Paramyxovirus |
| | MAELTSNITVLNLNLNQLTNKIQREIIPRITLIDTATTITIPSAITYILATLT | G Protein (No |
| | TRISELLPSINQKCEFKTPTLVLNDCRINCTPPLNPSDGVKMSSLATNL | Met) |
| | VAHGPSPCRNFSSVPTIYYYRIPGLYNRTALDERCILNPRLTISSTKFAY | |
| | VHSEYDKNCTRGFKYYELMTFGEILEGPEKEPRMFSRSFYSPTNAVNY | |
| | HSCTPIVTVNEGYFLCLECTSSDPLYKANLSNSTFHLVILRHNKDEKIV | |
| | SMPSFNLSTDQEYVQIIPAEGGGTAESGNLYFPCIGRLLHKRVTHPLCK | |
| | KSNCSRTDDESCLKSYYNQGSPQHQVVNCLIRIRNAQRDNPTWDVIT | |
| | VDLTNTYPGSRSRIFGSFSKPMLYQSSVSWHTLLQVAEITDLDKYQLD | |
| | WLDTPYISRPGGSECPFGNYCPTVCWEGTYNDVYSLTPNNDLFVTVY | |
| | LKSEQVAENPYFAIFSRDQILKEFPLDAWISSARTTTISCFMFNNEIWCI | |
| | AALEITRLNDDIIRPIYYSFWLPTDCRTPYPHTGKMTRVPLRSTYNY | |
| 10 | MATNRDNTITSAEVSQEDKVKKYYGVETAEKVADSISGNKVFILMNT | Mojiang virus, |
| | LLILTGAIITITLNITNLTAAKSQQNMLKIIQDDVNAKLEMFVNLDQLV | Tongguan 1 G |
| | KGEIKPKVSLINTAVSVSIPGQISNLQTKFLQKYVYLEESITKQCTCNPL | Protein |
| | SGIFPTSGPTYPPTDKPDDDTTDDDKVDTTIKPIEYPKPDGCNRTGDHF | |
| | TMEPGANFYTVPNLGPASSNSDECYTNPSFSIGSSIYMFSQEIRKTDCT | |
| | AGEILSIQIVLGRIVDKGQQGPQASPLLVWAVPNPKIINSCAVAAGDE | |
| | MGWVLCSVTLTAASGEPIPHMFDGFWLYKLEPDTEVVSYRITGYAYL | |
| | LDKQYDSVFIGKGGGIQKGNDLYFQMYGLSRNRQSFKALCEHGSCLG | |
| | TGGGGYQVLCDRAVMSFGSEESLITNAYLKVNDLASGKPVIIGQTFPP | |
| | SDSYKGSNGRMYTIGDKYGLYLAPSSWNRYLRFGITPDISVRSTTWLK | |
| | SQDPIMKILSTCTNTDRDMCPEICNTRGYQDIFPLSEDSEYYTYIGITPN | |
| | NGGTKNFVAVRDSDGHIASIDILQNYYSITSATISCFMYKDEIWCIAITE | |
| | GKKQKDNPQRIYAHSYKIRQMCYNMKSATVTVGNAKNITIRRY | |
| 11 | ATNRDNTITSAEVSQEDKVKKYYGVETAEKVADSISGNKVFILMNTL | Mojiang virus, |
| | LILTGAIITITLNITNLTAAKSQQNMLKIIQDDVNAKLEMFVNLDQLVK | Tongguan 1 G |
| | GEIKPKVSLINTAVSVSIPGQISNLQTKFLQKYVYLEESITKQCTCNPLS | (No Met) |
| | GIFPTSGPTYPPTDKPDDDTTDDDKVDTTIKPIEYPKPDGCNRTGDHFT | |
| | MEPGANFYTVPNLGPASSNSDECYTNPSFSIGSSIYMFSQEIRKTDCTA | |
| | GEILSIQIVLGRIVDKGQQGPQASPLLVWAVPNPKIINSCAVAAGDEM | |
| | GWVLCSVTLTAASGEPIPHMFDGFWLYKLEPDTEVVSYRITGYAYLL | |
| | DKQYDSVFIGKGGGIQKGNDLYFQMYGLSRNRQSFKALCEHGSCLGT | |
| | GGGGYQVLCDRAVMSFGSEESLITNAYLKVNDLASGKPVIIGQTFPPS | |
| | DSYKGSNGRMYTIGDKYGLYLAPSSWNRYLRFGITPDISVRSTTWLKS | |
| | QDPIMKILSTCTNTDRDMCPEICNTRGYQDIFPLSEDSEYYTYIGITPNN | |
| | GGTKNFVAVRDSDGHIASIDILQNYYSITSATISCFMYKDEIWCIAITEG | |
| | KKQKDNPQRIYAHSYKIRQMCYNMKSATVTVGNAKNITIRRY | |
| 12 | MKVR FENTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ5 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRENTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QCT | |
| 13 | MSKVIKSYY GTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated Δ20 |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRENTC PEICWEGVYN DAFLIDRINW | |
| | ISAGVELDSN QTAENPVFTV FKDNEILYRA QLASEDTNAQ | |
| | KTITNCFLLK NKIWCISLVE IYDTGDNVIR PKLFAVKIPE QCT | |
| 14 | MSYY GTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated Δ25 |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRENTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QCT | |
| 15 | ILHY EKLSKIGLVK GVTRKYKIKS NPLTKDIVIK MIPNVSNMSQ | Nipah virus |
| | CTGSVMENYK TRLNGILTPI KGALEIYKNQ THDLVGDVRL | NIV-F F0 |
| | AGVIMAGVAI GIATAAQITA GVALYEAMKN ADNINKLKSS | T234 |
| | IESTNEAVVK LQETAEKTVY VLTALQDYIN TNLVPTIDKI | truncation (aa |
| | SCKQTELSLD LALSKYLSDL LFVFGPNLQD PVSNSMTIQA | 525-544) AND |
| | ISQAFGGNYE TLLRTLGYAT EDFDDLLESD SITGQIIYVD | mutation on N- |
| | LSSYYIIVRV YFPILTEIQQ AYIQELLPVS FNNDNSEWIS IVPNFIL VRN | linked |
| | TLISNIEIGF CLITKRSVIC NQDYATPMTN NMRECLTGST | glycosylation |
| | EKCPRELVVS SHVPRFALSN GVLFANCISV TCQCQTTGRA | site |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNTGT | |
| 16 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion |
| | TRLNGILTPI KGALEIYKNN THDLVGDVRL AGVIMAGVAI | glycoprotein |
| | GIATAAQITA GVALYEAMKN ADNINKLKSS IESTNEAVVK | (FcDelta22) at |
| | LQETAEKTVY VLTALQDYIN TNLVPTIDKI SCKQTELSLD | cytoplasmic |
| | LALSKYLSDL LFVFGPNLQD PVSNSMTIQA ISQAFGGNYE | tail |
| | TLLRTLGYAT EDFDDLLESD SITGQIIYVD LSSYYIIVRV | (with signal |
| | YFPILTEIQQ AYIQELLPVS FNNDNSEWIS IVPNFILVRN TLISNIEIGF | sequence) |
| | CLITKRSVIC NQDYATPMTN NMRECLTGST EKCPRELVVS | |
| | SHVPRFALSN GVLFANCISV TCQCQTTGRA ISQSGEQTLL | |
| | MIDNTTCPTA VLGNVIISLG KYLGSVNYNS EGIAIGPPVF | |
| | TDKVDISSQI SSMNQSLQQS KDYIKEAQRL LDTVNPSLIS | |
| | MLSMIILYVL SIASLCIGLI TFISFIIVEK KRNT | |
| 17 | MKKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN ISCPNPLPFR | glycoprotein |
| | EYRPQTEGVS NLVGLPNNIC LQKTSNQILK PKLISYTLPV | Truncated and |
| | VGQSGTCITD PLLAMDEGYF AYSHLERIGS CSRGVSKQRI | mutated |
| | IGVGEVLDRG DEVPSLFMTN VWTPPNPNTV YHCSAVYNNE | (E501A, |
| | FYYVLCAVST VGDPILNSTY WSGSLMMTRL AVKPKSNGGG | W504A, |
| | YNQHQLALRS IEKGRYDKVM PYGPSGIKQG DTLYFPAVGF | Q530A, |
| | LVRTEFKYND SNCPITKCQY SKPENCRLSM GIRPNSHYIL | E533A) NiV G |
| | RSGLLKYNLS DGENPKVVFI EISDQRLSIG SPSKIYDSLG | protein (Gc Δ |
| | QPVFYQASFS WDTMIKFGDV LTVNPLVVNW RNNTVISRPG | 34) |
| | QSQCPRFNTC PAICAEGVYN DAFLIDRINW ISAGVFLDSN | NiVG protein |
| | ATAANPVFTV FKDNEILYRA QLASEDTNAQ KTITNCFLLK | |
| | NKIWCISLVE IYDTGDNVIR PKLFAVKIPE QCT | |
| 18 | KKINEGLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN ISCPNPLPFR | Truncated and |
| | EYRPQTEGVS NLVGLPNNIC LQKTSNQILK PKLISYTLPV | mutated |
| | VGQSGTCITD PLLAMDEGYF AYSHLERIGS CSRGVSKQRI | (E501 A, |
| | IGVGEVLDRG DEVPSLFMTN VWTPPNPNTV YHCSAVYNNE | W504A, |
| | FYYVLCAVST VGDPILNSTY WSGSLMMTRL AVKPKSNGGG | Q530A, |
| | YNQHQLALRS IEKGRYDKVM PYGPSGIKQG DTLYFPAVGF | E533A) NiV G |
| | LVRTEFKYND SNCPITKCQY SKPENCRLSM GIRPNSHYIL | protein (Gc Δ |
| | RSGLLKYNLS DGENPKVVFI EISDQRLSIG SPSKIYDSLG | 34) Without |
| | QPVFYQASFS WDTMIKFGDV LTVNPLVVNW RNNTVISRPG | N-terminal |
| | QSQCPRFNTC PAICAEGVYN DAFLIDRINW ISAGVFLDSN | methionine |
| | ATAANPVFTV FKDNEILYRA QLASEDTNAQ KTITNCFLLK | |
| | NKIWCISLVE IYDTGDNVIR PKLFAVKIPE QCT | |
| 19 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion |
| | TRLNGILTPI KGALEIYKNN THDLVGDVRL AGVIMAGVAI | glycoprotein |
| | GIATAAQITA GVALYEAMKN ADNINKLKSS IESTNEAVVK | (FcDelta22) at |
| | LQETAEKTVY VLTALQDYIN TNLVPTIDKI SCKQTELSLD | cytoplasmic |
| | LALSKYLSDL LFVFGPNLQD PVSNSMTIQA ISQAFGGNYE | tail |
| | TLLRTLGYAT EDFDDLLESD SITGQIIYVD LSSYYIIVRV | (with signal |
| | YFPILTEIQQ AYIQELLPVS FNNDNSEWIS IVPNFILVRN TLISNIEIGF | sequence) |
| | CLITKRSVIC NQDYATPMTN NMRECLTGST EKCPRELVVS | |
| | SHVPRFALSN GVLFANCISV TCQCQTTGRA ISQSGEQTLL | |
| | MIDNTTCPTA VLGNVIISLG KYLGSVNYNS EGIAIGPPVF | |
| | TDKVDISSQI SSMNQSLQQS KDYIKEAQRL LDTVNPSLIS | |
| | MLSMIILYVL SIASLCIGLI TFISFIIVEK KRNT | |
| 20 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NIV-F F0 |
| | AGVIMAGVAI GIATAAQITA GVALYEAMKN ADNINKLKSS | T234 |
| | IESTNEAVVK LQETAEKTVY VLTALQDYIN TNLVPTIDKI | truncation (aa |
| | SCKQTELSLD LALSKYLSDL LFVFGPNLQD PVSNSMTIQA | 525-544) |
| | ISQAFGGNYE TLLRTLGYAT EDFDDLLESD SITGQIIYVD | |
| | LSSYYIIVRV YFPILTEIQQ AYIQELLPVS FNNDNSEWIS IVPNFIL VRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNTGT | |
| 21 | ILHY EKLSKIGLVK GVTRKYKIKS NPLTKDIVIK MIPNVSNMSQ | Truncated |
| | CTGSVMENYK TRLNGILTPI KGALEIYKNN THDLVGDVRL | mature NiV |
| | AGVIMAGVAI GIATAAQITA GVALYEAMKN ADNINKLKSS | fusion |
| | IESTNEAVVK LQETAEKTVY VLTALQDYIN TNLVPTIDKI | glycoprotein |
| | SCKQTELSLD LALSKYLSDL LFVFGPNLQD PVSNSMTIQA | (FcDelta22) at |
| | ISQAFGGNYE TLLRTLGYAT EDFDDLLESD SITGQIIYVD | cytoplasmic |
| | LSSYYIIVRV YFPILTEIQQ AYIQELLPVS FNNDNSEWIS IVPNFIL VRN | tail |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 22 | FNTVIALLGS IVIIVMNIMI IQNYTRSTDN QAVIKDALQG | NivG protein |
| | IQQQIKGLAD KIGTEIGPKV SLIDTSSTIT IPANIGLLGS KISQSTASIN | attachment |
| | ENVNEKCKFT LPPLKIHECN ISCPNPLPFR EYRPQTEGVS | glycoprotein |
| | NLVGLPNNIC LQKTSNQILK PKLISYTLPV VGQSGTCITD | Without |
| | PLLAMDEGYF AYSHLERIGS CSRGVSKQRI IGVGEVLDRG | cytoplasmic |
| | DEVPSLFMTN VWTPPNPNTV YHCSAVYNNE FYYVLCAVST | tail |
| | VGDPILNSTY WSGSLMMTRL AVKPKSNGGG YNQHQLALRS | Uniprot |
| | IEKGRYDKVM PYGPSGIKQG DTLYFPAVGF LVRTEFKYND | Q9IH62 |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRENTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 23 | MMADSKLVSL NNNLSGKIKD QGKVIKNYYG TMDIKKINDG | Hendra virus |
| | LLDSKILGAF | G protein |
| | NTVIALLGSI IIIVMNIMII QNYTRTTDNQ ALIKESLQSV | Uniprot |
| | QQQIKALTDK IGTEIGPKVS LIDTSSTITI PANIGLLGSK ISQSTSSINE | O89343 |
| | NVNDKCKFTL | |
| | PPLKIHECNI SCPNPLPFRE YRPISQGVSD LVGLPNQICL | |
| | QKTTSTILKP RLISYTLPIN TREGVCITDP LLAVDNGFFA | |
| | YSHLEKIGSC TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ PVFYQASYSW DTMIKLGDVD TVDPLRVQWR | |
| | NNSVISRPGQ SQCPRFNVCP | |
| | EVCWEGTYND AFLIDRLNWV SAGVYLNSNQ TAENPVFAVF | |
| | KDNEILYQVP LAEDDTNAQK TITDCFLLEN VIWCISLVEI | |
| | YDTGDSVIRP KLFAVKIPAQ CSES | |
| 24 | MADSKLVSL NNNLSGKIKD QGKVIKNYYG TMDIKKINDG | Hendra virus |
| | LLDSKILGAF | G protein |
| | NTVIALLGSI IIIVMNIMII QNYTRTTDNQ ALIKESLQSV | Uniprot |
| | QQQIKALTDK IGTEIGPKVS LIDTSSTITI PANIGLLGSK ISQSTSSINE | O89343 |
| | NVNDKCKFTL | Without N- |
| | PPLKIHECNI SCPNPLPFRE YRPISQGVSD LVGLPNQICL | terminal |
| | QKTTSTILKP RLISYTLPIN TREGVCITDP LLAVDNGFFA | methionine |
| | YSHLEKIGSC TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ PVFYQASYSW DTMIKLGDVD TVDPLRVQWR | |
| | NNSVISRPGQ SQCPRFNVCP | |
| | EVCWEGTYND AFLIDRLNWV SAGVYLNSNQ TAENPVFAVF | |
| | KDNEILYQVP LAEDDTNAQK TITDCFLLEN VIWCISL VEI | |
| | YDTGDSVIRP KLFAVKIPAQ CSES | |
| 25 | FNTVIALLGSI IIIVMNIMII QNYTRTTDNQ ALIKESLQSV | Hendra virus |
| | QQQIKALTDK | G protein |
| | IGTEIGPKVS LIDTSSTITI PANIGLLGSK ISQSTSSINE NVNDKCKFTL | Uniprot |
| | PPLKIHECNI SCPNPLPFRE YRPISQGVSD LVGLPNQICL | O89343 |
| | QKTTSTILKP | Without |
| | RLISYTLPIN TREGVCITDP LLAVDNGFFA YSHLEKIGSC | cytoplasmic |
| | TRGIAKQRII | tail |
| | GVGEVLDRGD KVPSMFMTNV WTPPNPSTIH HCSSTYHEDF | |
| | YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ | |
| | PVFYQASYSW DTMIKLGDVD TVDPLRVQWR NNSVISRPGQ | |
| | SQCPRFNVCP EVCWEGTYND AFLIDRLNWV SAGVYLNSNQ | |
| | TAENPVFAVF KDNEILYQVP LAEDDTNAQK TITDCFLLEN | |
| | VIWCISLVEI YDTGDSVIRP KLFAVKIPAQ CSES | |
| 26 | FNTVIALLGSI IIIVMNIMII QNYTRTTDNQ ALIKESLQSV | Hendra virus |
| | QQQIKALTDK | G protein |
| | IGTEIGPKVS LIDTSSTITI PANIGLLGSK ISQSTSSINE NVNDKCKFTL | Uniprot |
| | PPLKIHECNI SCPNPLPFRE YRPISQGVSD LVGLPNQICL | O89343 |
| | QKTTSTILKP | Without |
| | RLISYTLPIN TREGVCITDP LLAVDNGFFA YSHLEKIGSC | cytoplasmic |
| | TRGIAKQRII | tail |
| | GVGEVLDRGD KVPSMFMTNV WTPPNPSTIH HCSSTYHEDF | |
| | YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ | |
| | PVFYQASYSW DTMIKLGDVD TVDPLRVQWR NNSVISRPGQ | |
| | SQCPRFNVCP EVCWEGTYND AFLIDRLNWV SAGVYLNSNQ | |
| | TAENPVFAVF KDNEILYQVP LAEDDTNAQK TITDCFLLEN | |
| | VIWCISLVEI YDTGDSVIRP KLFAVKIPAQ CSES | |
| 27 | MGPAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN ISCPNPLPFR | (602 aa) |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEIL YRA QLASEDTNAQ KTITNCFLLK | |
| | NKIWCISLVE IYDTGDNVIR PKLFAVKIPE QC | |
| 28 | MATQEVRLKCLLCGIIVLVLSLEGLGILHYEKLSKIGLVKGITRKYKIK | Hendra virus F |
| | SNPLTKDIVIKMIPNVSNVSKCTGTVMENYKSRLTGILSPIKGAIELYN | Protein |
| | NNTHDLVGDVKLAGVVMAGIAIGIATAAQITAGVALYEAMKNADNI | |
| | NKLKSSIESTNEAVVKLQETAEKTVYVLTALQDYINTNLVPTIDQISCK | |
| | QTELALDLALSKYLSDLLFVFGPNLQDPVSNSMTIQAISQAFGGNYET | |
| | LLRTLGYATEDFDDLLESDSIAGQIVYVDLSSYYIIVRVYFPILTEIQQA | |
| | YVQELLPVSFNNDNSEWISIVPNFVLIRNTLISNIEVKYCLITKKSVICN | |
| | QDYATPMTASVRECLTGSTDKCPRELVVSSHVPRFALSGGVLFANCIS | |
| | VTCQCQTTGRAISQSGEQTLLMIDNTTCTTVVLGNIIISLGKYLGSINY | |
| | NSESIAVGPPVYTDKVDISSQISSMNQSLQQSKDYIKEAQKILDTVNPS | |
| | LISMLSMIILYVLSIAALCIGLITFISFVIVEKKRGNYSRLDDRQVRPVSN | |
| | GDLYYIGT | |
| 29 | ILHYEKLSKIGLVKGITRKYKIKSNPLTKDIVIKMIPNVSNVSKCTGTV | Hendra virus F |
| | MENYKSRLTGILSPIKGAIELYNNNTHDLVGDVKLAGVVMAGIAIGIA | Protein, |
| | TAAQITAGVALYEAMKNADNINKLKSSIESTNEAVVKLQETAEKTVY | Without signal |
| | VLTALQDYINTNLVPTIDQISCKQTELALDLALSKYLSDLLFVFGPNLQ | sequence |
| | DPVSNSMTIQAISQAFGGNYETLLRTLGYATEDFDDLLESDSIAGQIVY | |
| | VDLSSYYIIVRVYFPILTEIQQAYVQELLPVSFNNDNSEWISIVPNFVLIR | |
| | NTLISNIEVKYCLITKKSVICNQDYATPMTASVRECLTGSTDKCPRELV | |
| | VSSHVPRFALSGGVLFANCISVTCQCQTTGRAISQSGEQTLLMIDNTTC | |
| | TTVVLGNIIISLGKYLGSINYNSESIAVGPPVYTDKVDISSQISSMNQSL | |
| | QQSKDYIKEAQKILDTVNPSLISMLSMIILYVLSIAALCIGLITFISFVIVE | |
| | KKRGNYSRLDDRQVRPVSNGDLYYIGT | |
| 30 | MVVILDKRCYCNLLILILMISECSVGILHYEKLSKIGLVKGVTRKYKIK | Nipah virus F |
| | SNPLTKDIVIKMIPNVSNMSQCTGSVMENYKTRLNGILTPIKGALEIYK | Protein |
| | NNTHDLVGDVRLAGVIMAGVAIGIATAAQITAGVALYEAMKNADNI | |
| | NKLKSSIESTNEAVVKLQETAEKTVYVLTALQDYINTNLVPTIDKISCK | |
| | QTELSLDLALSKYLSDLLFVFGPNLQDPVSNSMTIQAISQAFGGNYETL | |
| | LRTLGYATEDFDDLLESDSITGQIIYVDLSSYYIIVRVYFPILTEIQQAYI | |
| | QELLPVSFNNDNSEWISIVPNFILVRNTLISNIEIGFCLITKRSVICNQDY | |
| | ATPMTNNMRECLTGSTEKCPRELVVSSHVPRFALSNGVLFANCISVTC | |
| | QCQTTGRAISQSGEQTLLMIDNTTCPTAVLGNVIISLGKYLGSVNYNSE | |
| | GIAIGPPVFTDKVDISSQISSMNQSLQQSKDYIKEAQRLLDTVNPSLISM | |
| | LSMIILYVLSIASLCIGLITFISFIIVEKKRNTYSRLEDRRVRPTSSGDLYY | |
| | IGT | |
| 31 | ILHYEKLSKIGLVKGVTRKYKIKSNPLTKDIVIKMIPNVSNMSQCTGSV | Nipah virus F |
| | MENYKTRLNGILTPIKGALEIYKNNTHDLVGDVRLAGVIMAGVAIGIA | Protein, |
| | TAAQITAGVALYEAMKNADNINKLKSSIESTNEAVVKLQETAEKTVY | without signal |
| | VLTALQDYINTNLVPTIDKISCKQTELSLDLALSKYLSDLLFVFGPNLQ | sequence |
| | DPVSNSMTIQAISQAFGGNYETLLRTLGYATEDFDDLLESDSITGQIIY | |
| | VDLSSYYIIVRVYFPILTEIQQAYIQELLPVSFNNDNSEWISIVPNFILVR | |
| | NTLISNIEIGFCLITKRSVICNQDYATPMTNNMRECLTGSTEKCPRELV | |
| | VSSHVPRFALSNGVLFANCISVTCQCQTTGRAISQSGEQTLLMIDNTTC | |
| | PTAVLGNVIISLGKYLGSVNYNSEGIAIGPPVFTDKVDISSQISSMNQSL | |
| | QQSKDYIKEAQRLLDTVNPSLISMLSMIILYVLSIASLCIGLITFISFIIVE | |
| | KKRNTYSRLEDRRVRPTSSGDLYYIGT | |
| 32 | MSNKRTTVLIIISYTLFYLNNAAIVGFDFDKLNKIGVVQGRVLNYKIKG | Cedar Virus F |
| | DPMTKDLVLKFIPNIVNITECVREPLSRYNETVRRLLLPIHNMLGLYLN | Protein |
| | NTNAKMTGLMIAGVIMGGIAIGIATAAQITAGFALYEAKKNTENIQKL | |
| | TDSIMKTQDSIDKLTDSVGTSILILNKLQTYINNQLVPNLELLSCRQNKI | |
| | EFDLMLTKYLVDLMTVIGPNINNPVNKDMTIQSLSLLFDGNYDIMMS | |
| | ELGYTPQDFLDLIESKSITGQIIYVDMENLYVVIRTYLPTLIEVPDAQIY | |
| | EFNKITMSSNGGEYLSTIPNFILIRGNYMSNIDVATCYMTKASVICNQD | |
| | YSLPMSQNLRSCYQGETEYCPVEAVIASHSPRFALTNGVIFANCINTIC | |
| | RCQDNGKTITQNINQFVSMIDNSTCNDVMVDKFTIKVGKYMGRKDIN | |
| | NINIQIGPQIIIDKVDLSNEINKMNQSLKDSIFYLREAKRILDSVNISLISP | |
| | SVQLFLIIISVLSFIILLIIIVYLYCKSKHSYKYNKFIDDPDYYNDYKRERI | |
| | NGKASKSNNIYYVGD | |
| 33 | TVLIIISYTLFYLNNAAIVGFDFDKLNKIGVVQGRVLNYKIKGDPMTK | Cedar Virus F |
| | DLVLKFIPNIVNITECVREPLSRYNETVRRLLLPIHNMLGLYLNNTNAK | Protein, |
| | MTGLMIAGVIMGGIAIGIATAAQITAGFALYEAKKNTENIQKLTDSIM | without signal |
| | KTQDSIDKLTDSVGTSILILNKLQTYINNQLVPNLELLSCRQNKIEFDL | sequence |
| | MLTKYLVDLMTVIGPNINNPVNKDMTIQSLSLLFDGNYDIMMSELGY | |
| | TPQDFLDLIESKSITGQIIYVDMENLYVVIRTYLPTLIEVPDAQIYEFNKI | |
| | TMSSNGGEYLSTIPNFILIRGNYMSNIDVATCYMTKASVICNQDYSLP | |
| | MSQNLRSCYQGETEYCPVEAVIASHSPRFALTNGVIFANCINTICRCQD | |
| | NGKTITQNINQFVSMIDNSTCNDVMVDKFTIKVGKYMGRKDINNINIQ | |
| | IGPQIIIDKVDLSNEINKMNQSLKDSIFYLREAKRILDSVNISLISPSVQLF | |
| | LIIISVLSFIILLIIIVYLYCKSKHSYKYNKFIDDPDYYNDYKRERINGKA | |
| | SKSNNIYYVGD | |
| 34 | MALNKNMFSSLFLGYLLVYATTVQSSIHYDSLSKVGVIKGLTYNYKIK | Mojiang virus, |
| | GSPSTKLMVVKLIPNIDSVKNCTQKQYDEYKNLVRKALEPVKMAIDT | Tongguan 1 F |
| | MLNNVKSGNNKYRFAGAIMAGVALGVATAATVTAGIALHRSNENAQ | Protein |
| | AIANMKSAIQNTNEAVKQLQLANKQTLAVIDTIRGEINNNIIPVINQLS | |
| | CDTIGLSVGIRLTQYYSEIITAFGPALQNPVNTRITIQAISSVENGNFDEL | |
| | LKIMGYTSGDLYEILHSELIRGNIIDVDVDAGYIALEIEFPNLTLVPNAV | |
| | VQELMPISYNIDGDEWVTLVPRFVLTRTTLLSNIDTSRCTITDSSVICDN | |
| | DYALPMSHELIGCLQGDTSKCAREKVVSSYVPKFALSDGLVYANCLN | |
| | TICRCMDTDTPISQSLGATVSLLDNKRCSVYQVGDVLISVGSYLGDGE | |
| | YNADNVELGPPIVIDKIDIGNQLAGINQTLQEAEDYIEKSEEFLKGVNP | |
| | SIITLGSMVVLYIFMILIAIVSVIALVLSIKLTVKGNVVRQQFTYTQHVP | |
| | SMENINYVSH | |
| 35 | IHYDSLSKVGVIKGLTYNYKIKGSPSTKLMVVKLIPNIDSVKNCTQKQ | Mojiang virus, |
| | YDEYKNLVRKALEPVKMAIDTMLNNVKSGNNKYRFAGAIMAGVAL | Tongguan 1 F |
| | GVATAATVTAGIALHRSNENAQAIANMKSAIQNTNEAVKQLQLANK | Protein, |
| | QTLAVIDTIRGEINNNIIPVINQLSCDTIGLSVGIRLTQYYSEIITAFGPAL | without signal |
| | QNPVNTRITIQAISSVFNGNFDELLKIMGYTSGDLYEILHSELIRGNIIDV | sequence |
| | DVDAGYIALEIEFPNLTLVPNAVVQELMPISYNIDGDEWVTLVPRFVL | |
| | TRTTLLSNIDTSRCTITDSSVICDNDYALPMSHELIGCLQGDTSKCARE | |
| | KVVSSYVPKFALSDGLVYANCLNTICRCMDTDTPISQSLGATVSLLDN | |
| | KRCSVYQVGDVLISVGSYLGDGEYNADNVELGPPIVIDKIDIGNQLAG | |
| | INQTLQEAEDYIEKSEEFLKGVNPSIITLGSMVVLYIFMILIAIVSVIALV | |
| | LSIKLTVKGNVVRQQFTYTQHVPSMENINYVSH | |
| 36 | MKKKTDNPTISKRGHNHSRGIKSRALLRETDNYSNGLIVENLVRNCH | Bat |
| | HPSKNNLNYTKTQKRDSTIPYRVEERKGHYPKIKHLIDKSYKHIKRGK | Paramyxovirus |
| | RRNGHNGNIITIILLLILILKTQMSEGAIHYETLSKIGLIKGITREYKVKG | F Protein |
| | TPSSKDIVIKLIPNVTGLNKCTNISMENYKEQLDKILIPINNIIELYANST | |
| | KSAPGNARFAGVIIAGVALGVAAAAQITAGIALHEARQNAERINLLKD | |
| | SISATNNAVAELQEATGGIVNVITGMQDYINTNLVPQIDKLQCSQIKTA | |
| | LDISLSQYYSEILTVFGPNLQNPVTTSMSIQAISQSFGGNIDLLLNLLGY | |
| | TANDLLDLLESKSITGQITYINLEHYFMVIRVYYPIMTTISNAYVQELIK | |
| | ISFNVDGSEWVSLVPSYILIRNSYLSNIDISECLITKNSVICRHDFAMPM | |
| | SYTLKECLTGDTEKCPREAVVTSYVPRFAISGGVIYANCLSTTCQCYQ | |
| | TGKVIAQDGSQTLMMIDNQTCSIVRIEEILISTGKYLGSQEYNTMHVSV | |
| | GNPVFTDKLDITSQISNINQSIEQSKFYLDKSKAILDKINLNLIGSVPISIL | |
| | FIIAILSLILSIITFVIVMIIVRRYNKYTPLINSDPSSRRSTIQDVYIIPNPGE | |
| | HSIRSAARSIDRDRD | |
| 37 | SRALLRETDNYSNGLIVENLVRNCHHPSKNNLNYTKTQKRDSTIPYRV | Bat |
| | EERKGHYPKIKHLIDKSYKHIKRGKRRNGHNGNIITIILLLILILKTQMS | Paramyxovirus |
| | EGAIHYETLSKIGLIKGITREYKVKGTPSSKDIVIKLIPNVTGLNKCTNIS | F Protein, |
| | MENYKEQLDKILIPINNIIELYANSTKSAPGNARFAGVIIAGVALGVAA | without signal |
| | AAQITAGIALHEARQNAERINLLKDSISATNNAVAELQEATGGIVNVIT | sequence |
| | GMQDYINTNLVPQIDKLQCSQIKTALDISLSQYYSEILTVFGPNLQNPV | |
| | TTSMSIQAISQSFGGNIDLLLNLLGYTANDLLDLLESKSITGQITYINLE | |
| | HYFMVIRVYYPIMTTISNAYVQELIKISFNVDGSEWVSLVPSYILIRNSY | |
| | LSNIDISECLITKNSVICRHDFAMPMSYTLKECLTGDTEKCPREAVVTS | |
| | YVPRFAISGGVIYANCLSTTCQCYQTGKVIAQDGSQTLMMIDNQTCSI | |
| | VRIEEILISTGKYLGSQEYNTMHVSVGNPVFTDKLDITSQISNINQSIEQ | |
| | SKFYLDKSKAILDKINLNLIGSVPISILFIIAILSLILSIITFVIVMIIVRRYN | |
| | KYTPLINSDPSSRRSTIQDVYIIPNPGEHSIRSAARSIDRDRD | |
| 38 | MVVILDKRCYCNLLILILMISECSVG | signal |
| | | sequence |
| 39 | ILHYEKLSKIGLVKGVTRKYKIKSNPLTKDIVIKMIPNVSNMSQCTGSV | Nipah virus |
| | MENYKTRLNGILTPIKGALEIYKNNTHDLVGDVR | NiV-F F2 (aa |
| | | 27-109) |
| 40 | MVVILDKRCYCNLLILILMISECSVGILHYEKLSKIGLVKGVTRKYKIK | Nipah virus F |
| | SNPLTKDIVIKMIPNVSNMSQCTGSVMENYKTRLNGILTPIKGALEIYK | Protein |
| | NNTHDLVGDVRLAGVIMAGVAIGIATAAQITAGVALYEAMKNADNI | |
| | NKLKSSIESTNEAVVKLQETAEKTVYVLTALQDYINTNLVPTIDKISCK | |
| | QTELSLDLALSKYLSDLLFVFGPNLQDPVSNSMTIQAISQAFGGNYETL | |
| | LRTLGYATEDFDDLLESDSITGQIIYVDLSSYYIIVRVYFPILTEIQQAYI | |
| | QELLPVSFNNDNSEWISIVPNFILVRNTLISNIEIGFCLITKRSVICNQDY | |
| | ATPMTNNMRECLTGSTEKCPRELVVSSHVPRFALSNGVLFANCISVTC | |
| | QCQTTGRAISQSGEQTLLMIDNTTCPTAVLGNVIISLGKYLGSVNYNSE | |
| | GIAIGPPVFTDKVDISSQISSMNQSLQQSKDYIKEAQRLLDTVNPSLISM | |
| | LSMIILYVLSIASLCIGLITFISFIIVEKKRNTYSRLEDRRVRPTSSGDLYY | |
| | IGT | |
| 41 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NiV-F F0 (aa |
| | AGVIMAGVAI GIATAAQITA GVALYEAMKN ADNINKLKSS | 27-546) |
| | IESTNEAVVK LQETAEKTVY VLTALQDYIN TNLVPTIDKI | |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNTYSRLED | |
| | RRVRPTSSGD LYYIGT | |
| 42 | MKKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN ISCPNPLPFR | glycoprotein |
| | EYRPQTEGVS NLVGLPNNIC LQKTSNQILK PKLISYTLPV | Truncated (Gc |
| | VGQSGTCITD PLLAMDEGYF AYSHLERIGS CSRGVSKQRI | Δ34) |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QCT | |
| 43 | MTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated Δ30 |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRENTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QCT | |
| 44 | MGNTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ10 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRENTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 45 | MGKGK IPSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ15 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRENTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QC | |
| 46 | LAGVIMAGVAIGIATAAQITAGVALYEAMKNADNINKLKSSIESTNEA | Nipah virus |
| | VVKLQETAEKTVYVLTALQDYINTNLVPTIDKISCKQTELSLDLALSK | NIV F F1 (aa |
| | YLSDLLFVFGPNLQDPVSNSMTIQAISQAFGGNYETLLRTLGYATEDF | 110-546) |
| | DDLLESDSITGQIIYVDLSSYYIIVRVYFPILTEIQQAYIQELLPVSFNND | |
| | NSEWISIVPNFILVRNTLISNIEIGFCLITKRSVICNQDYATPMTNNMRE | |
| | CLTGSTEKCPRELVVSSHVPRFALSNGVLFANCISVTCQCQTTGRAISQ | |
| | SGEQTLLMIDNTTCPTAVLGNVIISLGKYLGSVNYNSEGIAIGPPVFTD | |
| | KVDISSQISSMNQSLQQSKDYIKEAQRLLDTVNPSLISMLSMIILYVLSI | |
| | ASLCIGLITFISFIIVEKKRNTYSRLEDRRVRPTSSGDLYYIGT | |
| 47 | GGCTTTACACTTTATGCTTCCGGCTCGTATGTTGTGTGGAATTGTGA | pMA2 CMV- |
| | GCGGATAACAATTTCACACAGGAAACAGCTATGACATGATTACGA | Cre |
| | ATTTCACAAATAAAGCATTTTTTTCACTGCATTCTAGTTGTGGTTTG | |
| | TCCAAACTCATCAATGTATCTTATCATGTCTGGATCAACTGGATAA | |
| | CTCAAGCTAACCAAAATCATCCCAAACTTCCCACCCCATACCCTAT | |
| | TACCACTGCCAATTACCTGTGGTTTCATTTACTCTAAACCTGTGATT | |
| | CCTCTGAATTATTTTCATTTTAAAGAAATTGTATTTGTTAAATATGT | |
| | ACTACAAACTTAGTAGTTGGAAGGGCTAATTCACTCCCAAAGAAG | |
| | ACAAGATATCCTTGATCTGTGGATCTACCACACACAAGGCTACTTC | |
| | CCTGATTAGCAGAACTACACACCAGGGCCAGGGGTCAGATATCCA | |
| | CTGACCTTTGGATGGTGCTACAAGCTAGTACCAGTTGAGCCAGATA | |
| | AGGTAGAAGAGGCCAATAAAGGAGAGAACACCAGCTTGTTACACC | |
| | CTGTGAGCCTGCATGGGATGGATGACCCGGAGAGAGAAGTGTTAG | |
| | AGTGGAGGTTTGACAGCCGCCTAGCATTTCATCACGTGGCCCGAG | |
| | AGCTGCATCCGGAGTACTTCAAGAACTGCTGATATCGAGCTTGCTA | |
| | CAAGGGACTTTCCGCTGGGGACTTTCCAGGGAGGCGTGGCCTGGG | |
| | CGGGACTGGGGAGTGGCGAGCCCTCAGATCCTGCATATAAGCAGC | |
| | TGCTTTTTGCCTGTACTGGGTCTCTCTGGTTAGACCAGATCTGAGCC | |
| | TGGGAGCTCTCTGGCTAACTAGGGAACCCACTGCTTAAGCCTCAAT | |
| | AAAGCTTGCCTTGAGTGCTTCAAGTAGTGTGTGCCCGTCTGTTGTG | |
| | TGACTCTGGTAACTAGAGATCCCTCAGACCCTTTTAGTCAGTGTGG | |
| | AAAATCTCTAGCAGTGGCGCCCGAACAGGGACTTGAAAGCGAAAG | |
| | GGAAACCAGAGGAGCTCTCTCGACGCAGGACTCGGCTTGCTGAAG | |
| | CGCGCACGGCAAGAGGCGAGGGGCGGCGACTGCAGAGTACGCCA | |
| | AAAATTTTGACTAGCGGAGGCTAGAAGGAGAGAGATGGGTGCGAG | |
| | AGCGTCAGTATTAAGCGGGGGAGAATTAGATCGCGATGGGAAAAA | |
| | ATTCGGTTAAGGCCAGGGGGAAAGAAAAAATATAAATTAAAACAT | |
| | ATAGTATGGGCAAGCAGGGAGCTAGAACGATTCGCAGTTAATCCT | |
| | GGCCTGTTAGAAACATCAGAAGGCTGTAGACAAATACTGGGACAG | |
| | CTACAACCATCCCTTCAGACAGGATCAGAAGAACTTAGATCATTAT | |
| | ATAATACAGTAGCAACCCTCTATTGTGTGCATCAAAGGATAGAGA | |
| | TAAAAGACACCAAGGAAGCTTTAGACAAGATAGAGGAAGAGCAA | |
| | AACAAAAGTAAGACCACCGCACAGCAAGCGGCCGGCCGCTGATCT | |
| | TCAGACCTGGAGGAGGAGATATGAGGGACAATTGGAGAAGTGAAT | |
| | TATATAAATATAAAGTAGTAAAAATTGAACCATTAGGAGTAGCAC | |
| | CCACCAAGGCAAAGAGAAGAGTGGTGCAGAGAGAAAAAAGAGCA | |
| | GTGGGAATAGGAGCTTTGTTCCTTGGGTTCTTGGGAGCAGCAGGA | |
| | AGCACTATGGGCGCAGCGTCAATGACGCTGACGGTACAGGCCAGA | |
| | CAATTATTGTCTGGTATAGTGCAGCAGCAGAACAATTTGCTGAGGG | |
| | CTATTGAGGCGCAACAGCATCTGTTGCAACTCACAGTCTGGGGCAT | |
| | CAAGCAGCTCCAGGCAAGAATCCTGGCTGTGGAAAGATACCTAAA | |
| | GGATCAACAGCTCCTGGGGATTTGGGGTTGCTCTGGAAAACTCATT | |
| | TGCACCACTGCTGTGCCTTGGAATGCTAGTTGGAGTAATAAATCTC | |
| | TGGAACAGATTTGGAATCACACGACCTGGATGGAGTGGGACAGAG | |
| | AAATTAACAATTACACAAGCTTAATACACTCCTTAATTGAAGAATC | |
| | GCAAAACCAGCAAGAAAAGAATGAACAAGAATTATTGGAATTAG | |
| | ATAAATGGGCAAGTTTGTGGAATTGGTTTAACATAACAAATTGGCT | |
| | GTGGTATATAAAATTATTCATAATGATAGTAGGAGGCTTGGTAGGT | |
| | TTAAGAATAGTTTTTGCTGTACTTTCTATAGTGAATAGAGTTAGGC | |
| | AGGGATATTCACCATTATCGTTTCAGACCCACCTCCCAACCCCGAG | |
| | GGGACCCGACAGGCCCGAAGGAATAGAAGAAGAAGGTGGAGAGA | |
| | GAGACAGAGACAGATCCATTCGATTAGTGAACGGATCTCGACGGT | |
| | ATCGCCGAATTCTCAACAATAGGCAGTATTCATCCACAATTTTAAA | |
| | AGAAAAGGGGGGATTGGGGGGTACAGTGCAGGGGAAAGAATAGT | |
| | AGACATAATAGCAACAGACATACAAACTAAAGAATTACAAAAACA | |
| | AATTACAAAAATTCAAAATTTTCGGGTTTATTACTCGGACAGCAGA | |
| | GATCCAGTTTGGACTAGTGGAGTTCCGCGTTACATAACTTACGCTA | |
| | AATGGCCCGCCTGGCTGACCGCCCAACGACCCCCGCCCATTGACGT | |
| | CAATAATGACGTATGTTCCCATAGTAACGCCAATAGGGACTTTCCA | |
| | TTGACGTCAATGGGTGGAGTATTTACGGTAAACTGCCCACTTGGCA | |
| | GTACATCAAGTGTATCATATGCCAAGTACGCCCCCTATTGACGTCA | |
| | ATGACGGTAAATGGCCCGCCTGGCATTATGCCCAGTACATGACCTT | |
| | ATGGGACTTTCCTACTTGGCAGTACATCTACGTATTAGTCATCGCT | |
| | ATTACCATGGTGATGCGGTTTTGGCAGTACATCAATGGGCGTGGAT | |
| | AGCGGTTTGACTCACGGGGATTTCCAAGTCTCCACCCCATTGACGT | |
| | CAATGGGAGTTTGTTTTGGCACCAAAATCAACGGGACTTTCCAAAA | |
| | TGTCGTAACAACTCCGCCCCATTGACGCAAATGGGCGGTAGGCGT | |
| | GTACGGTGGGAGGTCTATATAAGCAGAGCTCGTTTAGTGAACCGT | |
| | CAGATCGCCTGGAGACGCCATCCACGCTGTTTTGACCTCCATACAA | |
| | GACACCGGCGGCCGCCACCATGGTGCCCAAGAAGAAGAGGAAAG | |
| | TCTCCAACCTGCTGACTGTGCACCAAAACCTGCCTGCCCTCCCTGT | |
| | GGATGCCACCTCTGATGAAGTCAGGAAGAACCTGATGGACATGTT | |
| | CAGGGACAGGCAGGCCTTCTCTGAACACACCTGGAAGATGCTCCT | |
| | GTCTGTGTGCAGATCCTGGGCTGCCTGGTGCAAGCTGAACAACAG | |
| | GAAATGGTTCCCTGCTGAACCTGAGGATGTGAGGGACTACCTCCTG | |
| | TACCTGCAAGCCAGAGGCCTGGCTGTGAAGACCATCCAACAGCAC | |
| | CTGGGCCAGCTCAACATGCTGCACAGGAGATCTGGCCTGCCTCGCC | |
| | CTTCTGACTCCAATGCTGTGTCCCTGGTGATGAGGAGAATCAGAAA | |
| | GGAGAATGTGGATGCTGGGGAGAGAGCCAAGCAGGCCCTGGCCTT | |
| | TGAACGCACTGACTTTGACCAAGTCAGATCCCTGATGGAGAACTCT | |
| | GACAGATGCCAGGACATCAGGAACCTGGCCTTCCTGGGCATTGCC | |
| | TACAACACCCTGCTGCGCATTGCCGAAATTGCCAGAATCAGAGTG | |
| | AAGGACATCTCCCGCACCGATGGTGGGAGAATGCTGATCCACATT | |
| | GGCAGGACCAAGACCCTGGTGTCCACAGCTGGTGTGGAGAAGGCC | |
| | CTGTCCCTGGGGGTTACCAAGCTGGTGGAGAGATGGATCTCTGTGT | |
| | CTGGTGTGGCTGATGACCCCAACAACTACCTGTTCTGCCGGGTCAG | |
| | AAAGAATGGTGTGGCTGCCCCTTCTGCCACCTCCCAACTGTCCACC | |
| | CGGGCCCTGGAAGGGATCTTTGAGGCCACCCACCGCCTGATCTATG | |
| | GTGCCAAGGATGACTCTGGGCAGAGATACCTGGCCTGGTCTGGCC | |
| | ACTCTGCCAGAGTGGGTGCTGCCAGGGACATGGCCAGGGCTGGTG | |
| | TGTCCATCCCTGAAATCATGCAGGCTGGTGGCTGGACCAATGTGAA | |
| | CATTGTGATGAACTACATCAGAAACCTGGACTCTGAGACTGGGGC | |
| | CATGGTGAGGCTGCTCGAGGATGGGGACTGAGGATCCTAATCAAC | |
| | CTCTGGATTACAAAATTTGTGAAAGATTGACTGGTATTCTTAACTA | |
| | TGTTGCTCCTTTTACGCTATGTGGATACGCTGCTTTAATGCCTTTGT | |
| | ATCATGCTATTGCTTCCCGTATGGCTTTCATTTTCTCCTCCTTGTAT | |
| | AAATCCTGGTTGCTGTCTCTTTATGAGGAGTTGTGGCCCGTTGTCA | |
| | GGCAACGTGGCGTGGTGTGCACTGTGTTTGCTGACGCAACCCCCAC | |
| | TGGTTGGGGCATTGCCACCACCTGTCAGCTCCTTTCCGGGACTTTC | |
| | GCTTTCCCCCTCCCTATTGCCACGGCGGAACTCATCGCCGCCTGCC | |
| | TTGCCCGCTGCTGGACAGGGGCTCGGCTGTTGGGCACTGACAATTC | |
| | CGTGGTGTTGTCGGGGAAATCATCGTCCTTTCCTTGGCTGCTCGCC | |
| | TGTGTTGCCACCTGGATTCTGCGCGGGACGTCCTTCTGCTACGTCC | |
| | CTTCGGCCCTCAATCCAGCGGACCTTCCTTCCCGCGGCCTGCTGCC | |
| | GGCTCTGCGGCCTCTTCCGCGTCTTCGCCTTCGCCCTCAGACGAGT | |
| | CGGATCTCCCTTTGGGCCGCCTCCCCGCCTGGTACCTTTAAGACCA | |
| | ATGACTTACAAGGCAGCTGTAGATCTTAGCCACTTTTTAAAAGAAA | |
| | AGGGGGGACTGGAAGGGCTAATTCACTCCCAACGAAGACAAGATC | |
| | ACCTGCAGGACAGGCGCGCCCTGCTTTTTGCTTGTACTGGGTCTCT | |
| | CTGGTTAGACCAGATCTGAGCCTGGGAGCTCTCTGGCTAACTAGGG | |
| | AACCCACTGCTTAAGCCTCAATAAAGCTTGCCTTGAGTGCTTCAAG | |
| | TAGTGTGTGCCCGTCTGTTGTGTGACTCTGGTAACTAGAGATCCCT | |
| | CAGACCCTTTTAGTCAGTGTGGAAAATCTCTAGCACCCGGGCGATT | |
| | AAGGAAAGGGCTAGATCATTCTTGAAGACGAAAGGGCCTCGTGAT | |
| | ACGCCTATTTTTATAGGTTAATGTCATGATAATAATGGTTTCTTAGA | |
| | CGTCAGGTGGCACTTTTCGGGGAAATGTGCGCGGAACCCCTATTTG | |
| | TTTATTTTTCTAAATACATTCAAATATGTATCCGCTCATGAGACAAT | |
| | AACCCTGATAAATGCTTCAATAATATTGAAAAAGGAAGAGTATGA | |
| | GTATTCAACATTTCCGTGTCGCCCTTATTCCCTTTTTTGCGGCATTT | |
| | TGCCTTCCTGTTTTTGCTCACCCAGAAACGCTGGTGAAAGTAAAAG | |
| | ATGCTGAAGATCAGTTGGGTGCACGAGTGGGTTACATCGAACTGG | |
| | ATCTCAACAGCGGTAAGATCCTTGAGAGTTTTCGCCCCGAAGAAC | |
| | GTTTTCCAATGATGAGCACTTTTAAAGTTCTGCTATGTGGCGCGGT | |
| | ATTATCCCGTGTTGACGCCGGGCAAGAGCAACTCGGTCGCCGCAT | |
| | ACACTATTCTCAGAATGACTTGGTTGAGTACTCACCAGTCACAGAA | |
| | AAGCATCTTACGGATGGCATGACAGTAAGAGAATTATGCAGTGCT | |
| | GCCATAACCATGAGTGATAACACTGCGGCCAACTTACTTCTGACAA | |
| | CGATCGGAGGACCGAAGGAGCTAACCGCTTTTTTGCACAACATGG | |
| | GGGATCATGTAACTCGCCTTGATCGTTGGGAACCGGAGCTGAATG | |
| | AAGCCATACCAAACGACGAGCGTGACACCACGATGCCTGTAGCAA | |
| | TGGCAACAACGTTGCGCAAACTATTAACTGGCGAACTACTTACTCT | |
| | AGCTTCCCGGCAACAATTAATAGACTGGATGGAGGCGGATAAAGT | |
| | TGCAGGACCACTTCTGCGCTCGGCCCTTCCGGCTGGCTGGTTTATT | |
| | GCTGATAAATCTGGAGCCGGTGAGCGTGGGTCTCGCGGTATCATTG | |
| | CAGCACTGGGGCCAGATGGTAAGCCCTCCCGTATCGTAGTTATCTA | |
| | CACGACGGGGAGTCAGGCAACTATGGATGAACGAAATAGACAGAT | |
| | CGCTGAGATAGGTGCCTCACTGATTAAGCATTGGTAACTGTCAGAC | |
| | CAAGTTTACTCATATATACTTTAGATTGATTTAAAACTTCATTTTTA | |
| | ATTTAAAAGGATCTAGGTGAAGATCCTTTTTGATAATCTCATGACC | |
| | AAAATCCCTTAACGTGAGTTTTCGTTCCACTGAGCGTCAGACCCCG | |
| | TAGAAAAGATCAAAGGATCTTCTTGAGATCCTTTTTTTCTGCGCGT | |
| | AATCTGCTGCTTGCAAACAAAAAAACCACCGCTACCAGCGGTGGT | |
| | TTGTTTGCCGGATCAAGAGCTACCAACTCTTTTTCCGAAGGTAACT | |
| | GGCTTCAGCAGAGCGCAGATACCAAATACTGTTCTTCTAGTGTAGC | |
| | CGTAGTTAGGCCACCACTTCAAGAACTCTGTAGCACCGCCTACATA | |
| | CCTCGCTCTGCTAATCCTGTTACCAGTGGCTGCTGCCAGTGGCGAT | |
| | AAGTCGTGTCTTACCGGGTTGGACTCAAGACGATAGTTACCGGATA | |
| | AGGCGCAGCGGTCGGGCTGAACGGGGGGTTCGTGCACACAGCCCA | |
| | GCTTGGAGCGAACGACCTACACCGAACTGAGATACCTACAGCGTG | |
| | AGCTATGAGAAAGCGCCACGCTTCCCGAAGGGAGAAAGGCGGAC | |
| | AGGTATCCGGTAAGCGGCAGGGTCGGAACAGGAGAGCGCACGAG | |
| | GGAGCTTCCAGGGGGAAACGCCTGGTATCTTTATAGTCCTGTCGGG | |
| | TTTCGCCACCTCTGACTTGAGCGTCGATTTTTGTGATGCTCGTCAGG | |
| | GGGGCGGAGCCTATGGAAAAACGCCAGCAACGCGGCCTTTTTACG | |
| | GTTCCTGGCCTTTTGCTGGCCTTTTGCTCACATGTTCTTTCCTGCGT | |
| | TATCCCCTGATTCTGTGGATAACCGTATTACCGCCTTTGAGTGAGC | |
| | TGATACCGCTCGCCGCAGCCGAACGACCGAGCGCAGCGAGTCAGT | |
| | GAGCGAGGAAGCGGAAGAGCGCCCAATACGCAAACCGCCTCTCCC | |
| | CGCGCGTTGGCCGATTCATTAATGCAGCAAGCTCATGGCTGACTAA | |
| | TTTTTTTTATTTATGCAGAGGCCGAGGCCGCCTCGGCCTCTGAGCT | |
| | ATTCCAGAAGTAGTGAGGAGGCTTTTTTGGAGGCCTAGGCTTTTGC | |
| | AAAAAGCTCCCCGTGGCACGACAGGTTTCCCGACTGGAAAGCGGG | |
| | CAGTGAGCGCAACGCAATTAATGTGAGTTAGCTCACTCATTAGGC | |
| | ACCCCA | |
| 48 | TGGAAGGGCTAATTCACTCCCAAAGAAGACAAGATATCCTTGATC | pHAGE with |
| | TGTGGATCTACCACACACAAGGCTACTTCCCTGATTAGCAGAACTA | Cre starting at |
| | CACACCAGGGCCAGGGGTCAGATATCCACTGACCTTTGGATGGTG | the GAG start |
| | CTACAAGCTAGTACCAGTTGAGCCAGATAAGGTAGAAGAGGCCAA | codon |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGCAGAGTACGCCAAAAATTTTGACTAGCGG | |
| | AGGCTAGAAGGAGAGAGATGGTGCCCAAGAAGAAGAGGAAAGTC | |
| | TCCAACCTGCTGACTGTGCACCAAAACCTGCCTGCCCTCCCTGTGG | |
| | ATGCCACCTCTGATGAAGTCAGGAAGAACCTGATGGACATGTTCA | |
| | GGGACAGGCAGGCCTTCTCTGAACACACCTGGAAGATGCTCCTGT | |
| | CTGTGTGCAGATCCTGGGCTGCCTGGTGCAAGCTGAACAACAGGA | |
| | AATGGTTCCCTGCTGAACCTGAGGATGTGAGGGACTACCTCCTGTA | |
| | CCTGCAAGCCAGAGGCCTGGCTGTGAAGACCATCCAACAGCACCT | |
| | GGGCCAGCTCAACATGCTGCACAGGAGATCTGGCCTGCCTCGCCCT | |
| | TCTGACTCCAATGCTGTGTCCCTGGTGATGAGGAGAATCAGAAAG | |
| | GAGAATGTGGATGCTGGGGAGAGAGCCAAGCAGGCCCTGGCCTTT | |
| | GAACGCACTGACTTTGACCAAGTCAGATCCCTGATGGAGAACTCT | |
| | GACAGATGCCAGGACATCAGGAACCTGGCCTTCCTGGGCATTGCC | |
| | TACAACACCCTGCTGCGCATTGCCGAAATTGCCAGAATCAGAGTG | |
| | AAGGACATCTCCCGCACCGATGGTGGGAGAATGCTGATCCACATT | |
| | GGCAGGACCAAGACCCTGGTGTCCACAGCTGGTGTGGAGAAGGCC | |
| | CTGTCCCTGGGGGTTACCAAGCTGGTGGAGAGATGGATCTCTGTGT | |
| | CTGGTGTGGCTGATGACCCCAACAACTACCTGTTCTGCCGGGTCAG | |
| | AAAGAATGGTGTGGCTGCCCCTTCTGCCACCTCCCAACTGTCCACC | |
| | CGGGCCCTGGAAGGGATCTTTGAGGCCACCCACCGCCTGATCTATG | |
| | GTGCCAAGGATGACTCTGGGCAGAGATACCTGGCCTGGTCTGGCC | |
| | ACTCTGCCAGAGTGGGTGCTGCCAGGGACATGGCCAGGGCTGGTG | |
| | TGTCCATCCCTGAAATCATGCAGGCTGGTGGCTGGACCAATGTGAA | |
| | CATTGTGATGAACTACATCAGAAACCTGGACTCTGAGACTGGGGC | |
| | CATGGTGAGGCTGCTCGAGGATGGGGACTGAGGATCCTAATCAAC | |
| | CTCTGGATTACAAAATTTGTGAAAGATTGACTGGTATTCTTAACTA | |
| | TGTTGCTCCTTTTACGCTATGTGGATACGCTGCTTTAATGCCTTTGT | |
| | ATCATGCTATTGCTTCCCGTATGGCTTTCATTTTCTCCTCCTTGTAT | |
| | AAATCCTGGTTGCTGTCTCTTTATGAGGAGTTGTGGCCCGTTGTCA | |
| | GGCAACGTGGCGTGGTGTGCACTGTGTTTGCTGACGCAACCCCCAC | |
| | TGGTTGGGGCATTGCCACCACCTGTCAGCTCCTTTCCGGGACTTTC | |
| | GCTTTCCCCCTCCCTATTGCCACGGCGGAACTCATCGCCGCCTGCC | |
| | TTGCCCGCTGCTGGACAGGGGCTCGGCTGTTGGGCACTGACAATTC | |
| | CGTGGTGTTGTCGGGGAAATCATCGTCCTTTCCTTGGCTGCTCGCC | |
| | TGTGTTGCCACCTGGATTCTGCGCGGGACGTCCTTCTGCTACGTCC | |
| | CTTCGGCCCTCAATCCAGCGGACCTTCCTTCCCGCGGCCTGCTGCC | |
| | GGCTCTGCGGCCTCTTCCGCGTCTTCGCCTTCGCCCTCAGACGAGT | |
| | CGGATCTCCCTTTGGGCCGCCTCCCCGCCTGAGATCCTTTAAGACC | |
| | AATGACTTACAAGGCAGCTGTAGATCTTAGCCACTTTTTAAAAGAA | |
| | AAGGGGGGACTGGAAGGGCTAATTCACTCCCAACGAAGACAAGAT | |
| | CTGCTTTTTGCTTGTACTGGGTCTCTCTGGTTAGACCAGATCTGAGC | |
| | CTGGGAGCTCTCTGGCTAACTAGGGAACCCACTGCTTAAGCCTCAA | |
| | TAAAGCTTGCCTTGAGTGCTTCAAGTAGTGTGTGCCCGTCTGTTGT | |
| | GTGACTCTGGTAACTAGAGATCCCTCAGACCCTTTTAGTCAGTGTG | |
| | GAAAATCTCTAGCAGTAGTAGTTCATGTCATCTTATTATTCAGTATT | |
| | TATAACTTGCAAAGAAATGAATATCAGAGAGTGAGAGGCCCGGGT | |
| | TAATTAAGGAAAGGGCTAGATCATTCTTGAAGACGAAAGGGCCTC | |
| | GTGATACGCCTATTTTTATAGGTTAATGTCATGATAATAATGGTTTC | |
| | TTAGACGTCAGGTGGCACTTTTCGGGGAAATGTGCGCGGAACCCCT | |
| | ATTTGTTTATTTTTCTAAATACATTCAAATATGTATCCGCTCATGAG | |
| | ACAATAACCCTGATAAATGCTTCAATAATATTGAAAAAGGAAGAG | |
| | TATGAGTATTCAACATTTCCGTGTCGCCCTTATTCCCTTTTTTGCGG | |
| | CATTTTGCCTTCCTGTTTTTGCTCACCCAGAAACGCTGGTGAAAGT | |
| | AAAAGATGCTGAAGATCAGTTGGGTGCACGAGTGGGTTACATCGA | |
| | ACTGGATCTCAACAGCGGTAAGATCCTTGAGAGTTTTCGCCCCGAA | |
| | GAACGTTTTCCAATGATGAGCACTTTTAAAGTTCTGCTATGTGGCG | |
| | CGGTATTATCCCGTGTTGACGCCGGGCAAGAGCAACTCGGTCGCC | |
| | GCATACACTATTCTCAGAATGACTTGGTTGAGTACTCACCAGTCAC | |
| | AGAAAAGCATCTTACGGATGGCATGACAGTAAGAGAATTATGCAG | |
| | TGCTGCCATAACCATGAGTGATAACACTGCGGCCAACTTACTTCTG | |
| | ACAACGATCGGAGGACCGAAGGAGCTAACCGCTTTTTTGCACAAC | |
| | ATGGGGGATCATGTAACTCGCCTTGATCGTTGGGAACCGGAGCTG | |
| | AATGAAGCCATACCAAACGACGAGCGTGACACCACGATGCCTGTA | |
| | GCAATGGCAACAACGTTGCGCAAACTATTAACTGGCGAACTACTT | |
| | ACTCTAGCTTCCCGGCAACAATTAATAGACTGGATGGAGGCGGAT | |
| | AAAGTTGCAGGACCACTTCTGCGCTCGGCCCTTCCGGCTGGCTGGT | |
| | TTATTGCTGATAAATCTGGAGCCGGTGAGCGTGGGTCTCGCGGTAT | |
| | CATTGCAGCACTGGGGCCAGATGGTAAGCCCTCCCGTATCGTAGTT | |
| | ATCTACACGACGGGGAGTCAGGCAACTATGGATGAACGAAATAGA | |
| | CAGATCGCTGAGATAGGTGCCTCACTGATTAAGCATTGGTAACTGT | |
| | CAGACCAAGTTTACTCATATATACTTTAGATTGATTTAAAACTTCA | |
| | TTTTTAATTTAAAAGGATCTAGGTGAAGATCCTTTTTGATAATCTCA | |
| | TGACCAAAATCCCTTAACGTGAGTTTTCGTTCCACTGAGCGTCAGA | |
| | CCCCGTAGAAAAGATCAAAGGATCTTCTTGAGATCCTTTTTTTCTG | |
| | CGCGTAATCTGCTGCTTGCAAACAAAAAAACCACCGCTACCAGCG | |
| | GTGGTTTGTTTGCCGGATCAAGAGCTACCAACTCTTTTTCCGAAGG | |
| | TAACTGGCTTCAGCAGAGCGCAGATACCAAATACTGTTCTTCTAGT | |
| | GTAGCCGTAGTTAGGCCACCACTTCAAGAACTCTGTAGCACCGCCT | |
| | ACATACCTCGCTCTGCTAATCCTGTTACCAGTGGCTGCTGCCAGTG | |
| | GCGATAAGTCGTGTCTTACCGGGTTGGACTCAAGACGATAGTTACC | |
| | GGATAAGGCGCAGCGGTCGGGCTGAACGGGGGGTTCGTGCACACA | |
| | GCCCAGCTTGGAGCGAACGACCTACACCGAACTGAGATACCTACA | |
| | GCGTGAGCTATGAGAAAGCGCCACGCTTCCCGAAGGGAGAAAGGC | |
| | GGACAGGTATCCGGTAAGCGGCAGGGTCGGAACAGGAGAGCGCA | |
| | CGAGGGAGCTTCCAGGGGGAAACGCCTGGTATCTTTATAGTCCTGT | |
| | CGGGTTTCGCCACCTCTGACTTGAGCGTCGATTTTTGTGATGCTCGT | |
| | CAGGGGGGCGGAGCCTATGGAAAAACGCCAGCAACGCGGCCTTTT | |
| | TACGGTTCCTGGCCTTTTGCTGGCCTTTTGCTCACATGTTCTTTCCT | |
| | GCGTTATCCCCTGATTCTGTGGATAACCGTATTACCGCCTTTGAGT | |
| | GAGCTGATACCGCTCGCCGCAGCCGAACGACCGAGCGCAGCGAGT | |
| | CAGTGAGCGAGGAAGCGGAAGAGCGCCCAATACGCAAACCGCCTC | |
| | TCCCCGCGCGTTGGCCGATTCATTAATGCAGCAAGCTCATGGCTGA | |
| | CTAATTTTTTTTATTTATGCAGAGGCCGAGGCCGCCTCGGCCTCTG | |
| | AGCTATTCCAGAAGTAGTGAGGAGGCTTTTTTGGAGGCCTAGGCTT | |
| | TTGCAAAAAGCTCCCCGTGGCACGACAGGTTTCCCGACTGGAAAG | |
| | CGGGCAGTGAGCGCAACGCAATTAATGTGAGTTAGCTCACTCATT | |
| | AGGCACCCCAGGCTTTACACTTTATGCTTCCGGCTCGTATGTTGTG | |
| | TGGAATTGTGAGCGGATAACAATTTCACACAGGAAACAGCTATGA | |
| | CATGATTACGAATTTCACAAATAAAGCATTTTTTTCACTGCATTCT | |
| | AGTTGTGGTTTGTCCAAACTCATCAATGTATCTTATCATGTCTGGAT | |
| | CAACTGGATAACTCAAGCTAACCAAAATCATCCCAAACTTCCCACC | |
| | CCATACCCTATTACCACTGCCAATTACCTGTGGTTTCATTTACTCTA | |
| | AACCTGTGATTCCTCTGAATTATTTTCATTTTAAAGAAATTGTATTT | |
| | GTTAAATATGTACTACAAACTTAGTAGT | |
| 49 | TGGAAGGGCTAATTCACTCCCAAAGAAGACAAGATATCCTTGATC | pMA2 CMV- |
| | TGTGGATCTACCACACACAAGGCTACTTCCCTGATTAGCAGAACTA | EGFP-IRES- |
| | CACACCAGGGCCAGGGGTCAGATATCCACTGACCTTTGGATGGTG | Cre |
| | CTACAAGCTAGTACCAGTTGAGCCAGATAAGGTAGAAGAGGCCAA | |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGCAGAGTACGCCAAAAATTTTGACTAGCGG | |
| | AGGCTAGAAGGAGAGAGATGGGTGCGAGAGCGTCAGTATTAAGC | |
| | GGGGGAGAATTAGATCGCGATGGGAAAAAATTCGGTTAAGGCCAG | |
| | GGGGAAAGAAAAAATATAAATTAAAACATATAGTATGGGCAAGC | |
| | AGGGAGCTAGAACGATTCGCAGTTAATCCTGGCCTGTTAGAAACA | |
| | TCAGAAGGCTGTAGACAAATACTGGGACAGCTACAACCATCCCTT | |
| | CAGACAGGATCAGAAGAACTTAGATCATTATATAATACAGTAGCA | |
| | ACCCTCTATTGTGTGCATCAAAGGATAGAGATAAAAGACACCAAG | |
| | GAAGCTTTAGACAAGATAGAGGAAGAGCAAAACAAAAGTAAGAC | |
| | CACCGCACAGCAAGCGGCCGGCCGCTGATCTTCAGACCTGGAGGA | |
| | GGAGATATGAGGGACAATTGGAGAAGTGAATTATATAAATATAAA | |
| | GTAGTAAAAATTGAACCATTAGGAGTAGCACCCACCAAGGCAAAG | |
| | AGAAGAGTGGTGCAGAGAGAAAAAAGAGCAGTGGGAATAGGAGC | |
| | TTTGTTCCTTGGGTTCTTGGGAGCAGCAGGAAGCACTATGGGCGCA | |
| | GCGTCAATGACGCTGACGGTACAGGCCAGACAATTATTGTCTGGT | |
| | ATAGTGCAGCAGCAGAACAATTTGCTGAGGGCTATTGAGGCGCAA | |
| | CAGCATCTGTTGCAACTCACAGTCTGGGGCATCAAGCAGCTCCAG | |
| | GCAAGAATCCTGGCTGTGGAAAGATACCTAAAGGATCAACAGCTC | |
| | CTGGGGATTTGGGGTTGCTCTGGAAAACTCATTTGCACCACTGCTG | |
| | TGCCTTGGAATGCTAGTTGGAGTAATAAATCTCTGGAACAGATTTG | |
| | GAATCACACGACCTGGATGGAGTGGGACAGAGAAATTAACAATTA | |
| | CACAAGCTTAATACACTCCTTAATTGAAGAATCGCAAAACCAGCA | |
| | AGAAAAGAATGAACAAGAATTATTGGAATTAGATAAATGGGCAAG | |
| | TTTGTGGAATTGGTTTAACATAACAAATTGGCTGTGGTATATAAAA | |
| | TTATTCATAATGATAGTAGGAGGCTTGGTAGGTTTAAGAATAGTTT | |
| | TTGCTGTACTTTCTATAGTGAATAGAGTTAGGCAGGGATATTCACC | |
| | ATTATCGTTTCAGACCCACCTCCCAACCCCGAGGGGACCCGACAG | |
| | GCCCGAAGGAATAGAAGAAGAAGGTGGAGAGAGAGACAGAGACA | |
| | GATCCATTCGATTAGTGAACGGATCTCGACGGTATCGCCGAATTCT | |
| | CAACAATAGGCAGTATTCATCCACAATTTTAAAAGAAAAGGGGGG | |
| | ATTGGGGGGTACAGTGCAGGGGAAAGAATAGTAGACATAATAGCA | |
| | ACAGACATACAAACTAAAGAATTACAAAAACAAATTACAAAAATT | |
| | CAAAATTTTCGGGTTTATTACTCGGACAGCAGAGATCCAGTTTGGA | |
| | CTAGTGGAGTTCCGCGTTACATAACTTACGCTAAATGGCCCGCCTG | |
| | GCTGACCGCCCAACGACCCCCGCCCATTGACGTCAATAATGACGT | |
| | ATGTTCCCATAGTAACGCCAATAGGGACTTTCCATTGACGTCAATG | |
| | GGTGGAGTATTTACGGTAAACTGCCCACTTGGCAGTACATCAAGTG | |
| | TATCATATGCCAAGTACGCCCCCTATTGACGTCAATGACGGTAAAT | |
| | GGCCCGCCTGGCATTATGCCCAGTACATGACCTTATGGGACTTTCC | |
| | TACTTGGCAGTACATCTACGTATTAGTCATCGCTATTACCATGGTG | |
| | ATGCGGTTTTGGCAGTACATCAATGGGCGTGGATAGCGGTTTGACT | |
| | CACGGGGATTTCCAAGTCTCCACCCCATTGACGTCAATGGGAGTTT | |
| | GTTTTGGCACCAAAATCAACGGGACTTTCCAAAATGTCGTAACAAC | |
| | TCCGCCCCATTGACGCAAATGGGCGGTAGGCGTGTACGGTGGGAG | |
| | GTCTATATAAGCAGAGCTCGTTTAGTGAACCGTCAGATCGCCTGGA | |
| | GACGCCATCCACGCTGTTTTGACCTCCATACAAGACACCGGCGGCC | |
| | GCCATGGTGAGCAAGGGCGAGGAGCTGTTCACCGGGGTGGTGCCC | |
| | ATCCTGGTCGAGCTGGACGGCGACGTAAACGGCCACAAGTTCAGC | |
| | GTGTCCGGCGAGGGCGAGGGCGATGCCACCTACGGCAAGCTGACC | |
| | CTGAAGTTCATCTGCACCACCGGCAAGCTGCCCGTGCCCTGGCCCA | |
| | CCCTCGTGACCACCCTGACCTACGGCGTGCAGTGCTTCAGCCGCTA | |
| | CCCCGACCACATGAAGCAGCACGACTTCTTCAAGTCCGCCATGCCC | |
| | GAAGGCTACGTCCAGGAGCGCACCATCTTCTTCAAGGACGACGGC | |
| | AACTACAAGACCCGCGCCGAGGTGAAGTTCGAGGGCGACACCCTG | |
| | GTGAACCGCATCGAGCTGAAGGGCATCGACTTCAAGGAGGACGGC | |
| | AACATCCTGGGGCACAAGCTGGAGTACAACTACAACAGCCACAAC | |
| | GTCTATATCATGGCCGACAAGCAGAAGAACGGCATCAAGGTGAAC | |
| | TTCAAGATCCGCCACAACATCGAGGACGGCAGCGTGCAGCTCGCC | |
| | GACCACTACCAGCAGAACACCCCCATCGGCGACGGCCCCGTGCTG | |
| | CTGCCCGACAACCACTACCTGAGCACCCAGTCCGCCCTGAGCAAA | |
| | GACCCCAACGAGAAGCGCGATCACATGGTCCTGCTGGAGTTCGTG | |
| | ACCGCCGCCGGGATCACTCTCGGCATGGACGAGCTGTACAAGTAA | |
| | GGATCCCTCCCCCCCCCCTAACGTTACTGGCCGAAGCCGCTTGGAA | |
| | TAAGGCCGGTGTGCGTTTGTCTATATGTTATTTTCCACCATATTGCC | |
| | GTCTTTTGGCAATGTGAGGGCCCGGAAACCTGGCCCTGTCTTCTTG | |
| | ACGAGCATTCCTAGGGGTCTTTCCCCTCTCGCCAAAGGAATGCAAG | |
| | GTCTGTTGAATGTCGTGAAGGAAGCAGTTCCTCTGGAAGCTTCTTG | |
| | AAGACAAACAACGTCTGTAGCGACCCTTTGCAGGCAGCGGAACCC | |
| | CCCACCTGGCGACAGGTGCCTCTGCGGCCAAAAGCCACGTGTATA | |
| | AGATACACCTGCAAAGGCGGCACAACCCCAGTGCCACGTTGTGAG | |
| | TTGGATAGTTGTGGAAAGAGTCAAATGGCTCTCCTCAAGCGTATTC | |
| | AACAAGGGGCTGAAGGATGCCCAGAAGGTACCCCATTGTATGGGA | |
| | TCTGATCTGGGGCCTCGGTGCACATGCTTTACATGTGTTTAGTCGA | |
| | GGTTAAAAAACGTCTAGGCCCCCCGAACCACGGGGACGTGGTTTT | |
| | CCTTTGAAAAACACGATGATAATATGGCCACAACCATGGTGCCCA | |
| | AGAAGAAGAGGAAAGTCTCCAACCTGCTGACTGTGCACCAAAACC | |
| | TGCCTGCCCTCCCTGTGGATGCCACCTCTGATGAAGTCAGGAAGAA | |
| | CCTGATGGACATGTTCAGGGACAGGCAGGCCTTCTCTGAACACAC | |
| | CTGGAAGATGCTCCTGTCTGTGTGCAGATCCTGGGCTGCCTGGTGC | |
| | AAGCTGAACAACAGGAAATGGTTCCCTGCTGAACCTGAGGATGTG | |
| | AGGGACTACCTCCTGTACCTGCAAGCCAGAGGCCTGGCTGTGAAG | |
| | ACCATCCAACAGCACCTGGGCCAGCTCAACATGCTGCACAGGAGA | |
| | TCTGGCCTGCCTCGCCCTTCTGACTCCAATGCTGTGTCCCTGGTGAT | |
| | GAGGAGAATCAGAAAGGAGAATGTGGATGCTGGGGAGAGAGCCA | |
| | AGCAGGCCCTGGCCTTTGAACGCACTGACTTTGACCAAGTCAGATC | |
| | CCTGATGGAGAACTCTGACAGATGCCAGGACATCAGGAACCTGGC | |
| | CTTCCTGGGCATTGCCTACAACACCCTGCTGCGCATTGCCGAAATT | |
| | GCCAGAATCAGAGTGAAGGACATCTCCCGCACCGATGGTGGGAGA | |
| | ATGCTGATCCACATTGGCAGGACCAAGACCCTGGTGTCCACAGCT | |
| | GGTGTGGAGAAGGCCCTGTCCCTGGGGGTTACCAAGCTGGTGGAG | |
| | AGATGGATCTCTGTGTCTGGTGTGGCTGATGACCCCAACAACTACC | |
| | TGTTCTGCCGGGTCAGAAAGAATGGTGTGGCTGCCCCTTCTGCCAC | |
| | CTCCCAACTGTCCACCCGGGCCCTGGAAGGGATCTTTGAGGCCACC | |
| | CACCGCCTGATCTATGGTGCCAAGGATGACTCTGGGCAGAGATAC | |
| | CTGGCCTGGTCTGGCCACTCTGCCAGAGTGGGTGCTGCCAGGGAC | |
| | ATGGCCAGGGCTGGTGTGTCCATCCCTGAAATCATGCAGGCTGGTG | |
| | GCTGGACCAATGTGAACATTGTGATGAACTACATCAGAAACCTGG | |
| | ACTCTGAGACTGGGGCCATGGTGAGGCTGCTCGAGGATGGGGACT | |
| | GAATCGATAGATCCTAATCAACCTCTGGATTACAAAATTTGTGAAA | |
| | GATTGACTGGTATTCTTAACTATGTTGCTCCTTTTACGCTATGTGGA | |
| | TACGCTGCTTTAATGCCTTTGTATCATGCTATTGCTTCCCGTATGGC | |
| | TTTCATTTTCTCCTCCTTGTATAAATCCTGGTTGCTGTCTCTTTATGA | |
| | GGAGTTGTGGCCCGTTGTCAGGCAACGTGGCGTGGTGTGCACTGTG | |
| | TTTGCTGACGCAACCCCCACTGGTTGGGGCATTGCCACCACCTGTC | |
| | AGCTCCTTTCCGGGACTTTCGCTTTCCCCCTCCCTATTGCCACGGCG | |
| | GAACTCATCGCCGCCTGCCTTGCCCGCTGCTGGACAGGGGCTCGGC | |
| | TGTTGGGCACTGACAATTCCGTGGTGTTGTCGGGGAAATCATCGTC | |
| | CTTTCCTTGGCTGCTCGCCTGTGTTGCCACCTGGATTCTGCGCGGG | |
| | ACGTCCTTCTGCTACGTCCCTTCGGCCCTCAATCCAGCGGACCTTC | |
| | CTTCCCGCGGCCTGCTGCCGGCTCTGCGGCCTCTTCCGCGTCTTCGC | |
| | CTTCGCCCTCAGACGAGTCGGATCTCCCTTTGGGCCGCCTCCCCGC | |
| | CTGGTACCTTTAAGACCAATGACTTACAAGGCAGCTGTAGATCTTA | |
| | GCCACTTTTTAAAAGAAAAGGGGGGACTGGAAGGGCTAATTCACT | |
| | CCCAACGAAGACAAGATCACCTGCAGGACAGGCGCGCCCTGCTTT | |
| | TTGCTTGTACTGGGTCTCTCTGGTTAGACCAGATCTGAGCCTGGGA | |
| | GCTCTCTGGCTAACTAGGGAACCCACTGCTTAAGCCTCAATAAAGC | |
| | TTGCCTTGAGTGCTTCAAGTAGTGTGTGCCCGTCTGTTGTGTGACTC | |
| | TGGTAACTAGAGATCCCTCAGACCCTTTTAGTCAGTGTGGAAAATC | |
| | TCTAGCACCCGGGCGATTAAGGAAAGGGCTAGATCATTCTTGAAG | |
| | ACGAAAGGGCCTCGTGATACGCCTATTTTTATAGGTTAATGTCATG | |
| | ATAATAATGGTTTCTTAGACGTCAGGTGGCACTTTTCGGGGAAATG | |
| | TGCGCGGAACCCCTATTTGTTTATTTTTCTAAATACATTCAAATATG | |
| | TATCCGCTCATGAGACAATAACCCTGATAAATGCTTCAATAATATT | |
| | GAAAAAGGAAGAGTATGAGTATTCAACATTTCCGTGTCGCCCTTAT | |
| | TCCCTTTTTTGCGGCATTTTGCCTTCCTGTTTTTGCTCACCCAGAAA | |
| | CGCTGGTGAAAGTAAAAGATGCTGAAGATCAGTTGGGTGCACGAG | |
| | TGGGTTACATCGAACTGGATCTCAACAGCGGTAAGATCCTTGAGA | |
| | GTTTTCGCCCCGAAGAACGTTTTCCAATGATGAGCACTTTTAAAGT | |
| | TCTGCTATGTGGCGCGGTATTATCCCGTGTTGACGCCGGGCAAGAG | |
| | CAACTCGGTCGCCGCATACACTATTCTCAGAATGACTTGGTTGAGT | |
| | ACTCACCAGTCACAGAAAAGCATCTTACGGATGGCATGACAGTAA | |
| | GAGAATTATGCAGTGCTGCCATAACCATGAGTGATAACACTGCGG | |
| | CCAACTTACTTCTGACAACGATCGGAGGACCGAAGGAGCTAACCG | |
| | CTTTTTTGCACAACATGGGGGATCATGTAACTCGCCTTGATCGTTG | |
| | GGAACCGGAGCTGAATGAAGCCATACCAAACGACGAGCGTGACAC | |
| | CACGATGCCTGTAGCAATGGCAACAACGTTGCGCAAACTATTAAC | |
| | TGGCGAACTACTTACTCTAGCTTCCCGGCAACAATTAATAGACTGG | |
| | ATGGAGGCGGATAAAGTTGCAGGACCACTTCTGCGCTCGGCCCTTC | |
| | CGGCTGGCTGGTTTATTGCTGATAAATCTGGAGCCGGTGAGCGTGG | |
| | GTCTCGCGGTATCATTGCAGCACTGGGGCCAGATGGTAAGCCCTCC | |
| | CGTATCGTAGTTATCTACACGACGGGGAGTCAGGCAACTATGGAT | |
| | GAACGAAATAGACAGATCGCTGAGATAGGTGCCTCACTGATTAAG | |
| | CATTGGTAACTGTCAGACCAAGTTTACTCATATATACTTTAGATTG | |
| | ATTTAAAACTTCATTTTTAATTTAAAAGGATCTAGGTGAAGATCCT | |
| | TTTTGATAATCTCATGACCAAAATCCCTTAACGTGAGTTTTCGTTCC | |
| | ACTGAGCGTCAGACCCCGTAGAAAAGATCAAAGGATCTTCTTGAG | |
| | ATCCTTTTTTTCTGCGCGTAATCTGCTGCTTGCAAACAAAAAAACC | |
| | ACCGCTACCAGCGGTGGTTTGTTTGCCGGATCAAGAGCTACCAACT | |
| | CTTTTTCCGAAGGTAACTGGCTTCAGCAGAGCGCAGATACCAAATA | |
| | CTGTTCTTCTAGTGTAGCCGTAGTTAGGCCACCACTTCAAGAACTC | |
| | TGTAGCACCGCCTACATACCTCGCTCTGCTAATCCTGTTACCAGTG | |
| | GCTGCTGCCAGTGGCGATAAGTCGTGTCTTACCGGGTTGGACTCAA | |
| | GACGATAGTTACCGGATAAGGCGCAGCGGTCGGGCTGAACGGGGG | |
| | GTTCGTGCACACAGCCCAGCTTGGAGCGAACGACCTACACCGAAC | |
| | TGAGATACCTACAGCGTGAGCTATGAGAAAGCGCCACGCTTCCCG | |
| | AAGGGAGAAAGGCGGACAGGTATCCGGTAAGCGGCAGGGTCGGA | |
| | ACAGGAGAGCGCACGAGGGAGCTTCCAGGGGGAAACGCCTGGTAT | |
| | CTTTATAGTCCTGTCGGGTTTCGCCACCTCTGACTTGAGCGTCGATT | |
| | TTTGTGATGCTCGTCAGGGGGGCGGAGCCTATGGAAAAACGCCAG | |
| | CAACGCGGCCTTTTTACGGTTCCTGGCCTTTTGCTGGCCTTTTGCTC | |
| | ACATGTTCTTTCCTGCGTTATCCCCTGATTCTGTGGATAACCGTATT | |
| | ACCGCCTTTGAGTGAGCTGATACCGCTCGCCGCAGCCGAACGACC | |
| | GAGCGCAGCGAGTCAGTGAGCGAGGAAGCGGAAGAGCGCCCAAT | |
| | ACGCAAACCGCCTCTCCCCGCGCGTTGGCCGATTCATTAATGCAGC | |
| | AAGCTCATGGCTGACTAATTTTTTTTATTTATGCAGAGGCCGAGGC | |
| | CGCCTCGGCCTCTGAGCTATTCCAGAAGTAGTGAGGAGGCTTTTTT | |
| | GGAGGCCTAGGCTTTTGCAAAAAGCTCCCCGTGGCACGACAGGTT | |
| | TCCCGACTGGAAAGCGGGCAGTGAGCGCAACGCAATTAATGTGAG | |
| | TTAGCTCACTCATTAGGCACCCCAGGCTTTACACTTTATGCTTCCG | |
| | GCTCGTATGTTGTGTGGAATTGTGAGCGGATAACAATTTCACACAG | |
| | GAAACAGCTATGACATGATTACGAATTTCACAAATAAAGCATTTTT | |
| | TTCACTGCATTCTAGTTGTGGTTTGTCCAAACTCATCAATGTATCTT | |
| | ATCATGTCTGGATCAACTGGATAACTCAAGCTAACCAAAATCATCC | |
| | CAAACTTCCCACCCCATACCCTATTACCACTGCCAATTACCTGTGG | |
| | TTTCATTTACTCTAAACCTGTGATTCCTCTGAATTATTTTCATTTTA | |
| | AAGAAATTGTATTTGTTAAATATGTACTACAAACTTAGTAGT | |
| 50 | TGGAAGGGCTAATTCACTCCCAAAGAAGACAAGATATCCTTGATC | pMA2 IRES- |
| | TGTGGATCTACCACACACAAGGCTACTTCCCTGATTAGCAGAACTA | Cre (no |
| | CACACCAGGGCCAGGGGTCAGATATCCACTGACCTTTGGATGGTG | promoter) |
| | CTACAAGCTAGTACCAGTTGAGCCAGATAAGGTAGAAGAGGCCAA | |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGCAGAGTACGCCAAAAATTTTGACTAGCGG | |
| | AGGCTAGAAGGAGAGAGATGGGTGCGAGAGCGTCAGTATTAAGC | |
| | GGGGGAGAATTAGATCGCGATGGGAAAAAATTCGGTTAAGGCCAG | |
| | GGGGAAAGAAAAAATATAAATTAAAACATATAGTATGGGCAAGC | |
| | AGGGAGCTAGAACGATTCGCAGTTAATCCTGGCCTGTTAGAAACA | |
| | TCAGAAGGCTGTAGACAAATACTGGGACAGCTACAACCATCCCTT | |
| | CAGACAGGATCAGAAGAACTTAGATCATTATATAATACAGTAGCA | |
| | ACCCTCTATTGTGTGCATCAAAGGATAGAGATAAAAGACACCAAG | |
| | GAAGCTTTAGACAAGATAGAGGAAGAGCAAAACAAAAGTAAGAC | |
| | CACCGCACAGCAAGCGGCCGGCCGCTGATCTTCAGACCTGGAGGA | |
| | GGAGATATGAGGGACAATTGGAGAAGTGAATTATATAAATATAAA | |
| | GTAGTAAAAATTGAACCATTAGGAGTAGCACCCACCAAGGCAAAG | |
| | AGAAGAGTGGTGCAGAGAGAAAAAAGAGCAGTGGGAATAGGAGC | |
| | TTTGTTCCTTGGGTTCTTGGGAGCAGCAGGAAGCACTATGGGCGCA | |
| | GCGTCAATGACGCTGACGGTACAGGCCAGACAATTATTGTCTGGT | |
| | ATAGTGCAGCAGCAGAACAATTTGCTGAGGGCTATTGAGGCGCAA | |
| | CAGCATCTGTTGCAACTCACAGTCTGGGGCATCAAGCAGCTCCAG | |
| | GCAAGAATCCTGGCTGTGGAAAGATACCTAAAGGATCAACAGCTC | |
| | CTGGGGATTTGGGGTTGCTCTGGAAAACTCATTTGCACCACTGCTG | |
| | TGCCTTGGAATGCTAGTTGGAGTAATAAATCTCTGGAACAGATTTG | |
| | GAATCACACGACCTGGATGGAGTGGGACAGAGAAATTAACAATTA | |
| | CACAAGCTTAATACACTCCTTAATTGAAGAATCGCAAAACCAGCA | |
| | AGAAAAGAATGAACAAGAATTATTGGAATTAGATAAATGGGCAAG | |
| | TTTGTGGAATTGGTTTAACATAACAAATTGGCTGTGGTATATAAAA | |
| | TTATTCATAATGATAGTAGGAGGCTTGGTAGGTTTAAGAATAGTTT | |
| | TTGCTGTACTTTCTATAGTGAATAGAGTTAGGCAGGGATATTCACC | |
| | ATTATCGTTTCAGACCCACCTCCCAACCCCGAGGGGACCCGACAG | |
| | GCCCGAAGGAATAGAAGAAGAAGGTGGAGAGAGAGACAGAGACA | |
| | GATCCATTCGATTAGTGAACGGATCTCGACGGTATCGCCGAATTCT | |
| | CAACAATAGGCAGTATTCATCCACAATTTTAAAAGAAAAGGGGGG | |
| | ATTGGGGGGTACAGTGCAGGGGAAAGAATAGTAGACATAATAGCA | |
| | ACAGACATACAAACTAAAGAATTACAAAAACAAATTACAAAAATT | |
| | CAAAATTTTCGGGTTTATTACTCGGACAGCAGAGATCCAGTTTGGA | |
| | CTAGGATCCCTCCCCCCCCCCTAACGTTACTGGCCGAAGCCGCTTG | |
| | GAATAAGGCCGGTGTGCGTTTGTCTATATGTTATTTTCCACCATATT | |
| | GCCGTCTTTTGGCAATGTGAGGGCCCGGAAACCTGGCCCTGTCTTC | |
| | TTGACGAGCATTCCTAGGGGTCTTTCCCCTCTCGCCAAAGGAATGC | |
| | AAGGTCTGTTGAATGTCGTGAAGGAAGCAGTTCCTCTGGAAGCTTC | |
| | TTGAAGACAAACAACGTCTGTAGCGACCCTTTGCAGGCAGCGGAA | |
| | CCCCCCACCTGGCGACAGGTGCCTCTGCGGCCAAAAGCCACGTGT | |
| | ATAAGATACACCTGCAAAGGCGGCACAACCCCAGTGCCACGTTGT | |
| | GAGTTGGATAGTTGTGGAAAGAGTCAAATGGCTCTCCTCAAGCGT | |
| | ATTCAACAAGGGGCTGAAGGATGCCCAGAAGGTACCCCATTGTAT | |
| | GGGATCTGATCTGGGGCCTCGGTGCACATGCTTTACATGTGTTTAG | |
| | TCGAGGTTAAAAAACGTCTAGGCCCCCCGAACCACGGGGACGTGG | |
| | TTTTCCTTTGAAAAACACGATGATAATATGGCCACAACCATGGTGC | |
| | CCAAGAAGAAGAGGAAAGTCTCCAACCTGCTGACTGTGCACCAAA | |
| | ACCTGCCTGCCCTCCCTGTGGATGCCACCTCTGATGAAGTCAGGAA | |
| | GAACCTGATGGACATGTTCAGGGACAGGCAGGCCTTCTCTGAACA | |
| | CACCTGGAAGATGCTCCTGTCTGTGTGCAGATCCTGGGCTGCCTGG | |
| | TGCAAGCTGAACAACAGGAAATGGTTCCCTGCTGAACCTGAGGAT | |
| | GTGAGGGACTACCTCCTGTACCTGCAAGCCAGAGGCCTGGCTGTG | |
| | AAGACCATCCAACAGCACCTGGGCCAGCTCAACATGCTGCACAGG | |
| | AGATCTGGCCTGCCTCGCCCTTCTGACTCCAATGCTGTGTCCCTGG | |
| | TGATGAGGAGAATCAGAAAGGAGAATGTGGATGCTGGGGAGAGA | |
| | GCCAAGCAGGCCCTGGCCTTTGAACGCACTGACTTTGACCAAGTCA | |
| | GATCCCTGATGGAGAACTCTGACAGATGCCAGGACATCAGGAACC | |
| | TGGCCTTCCTGGGCATTGCCTACAACACCCTGCTGCGCATTGCCGA | |
| | AATTGCCAGAATCAGAGTGAAGGACATCTCCCGCACCGATGGTGG | |
| | GAGAATGCTGATCCACATTGGCAGGACCAAGACCCTGGTGTCCAC | |
| | AGCTGGTGTGGAGAAGGCCCTGTCCCTGGGGGTTACCAAGCTGGT | |
| | GGAGAGATGGATCTCTGTGTCTGGTGTGGCTGATGACCCCAACAA | |
| | CTACCTGTTCTGCCGGGTCAGAAAGAATGGTGTGGCTGCCCCTTCT | |
| | GCCACCTCCCAACTGTCCACCCGGGCCCTGGAAGGGATCTTTGAGG | |
| | CCACCCACCGCCTGATCTATGGTGCCAAGGATGACTCTGGGCAGA | |
| | GATACCTGGCCTGGTCTGGCCACTCTGCCAGAGTGGGTGCTGCCAG | |
| | GGACATGGCCAGGGCTGGTGTGTCCATCCCTGAAATCATGCAGGC | |
| | TGGTGGCTGGACCAATGTGAACATTGTGATGAACTACATCAGAAA | |
| | CCTGGACTCTGAGACTGGGGCCATGGTGAGGCTGCTCGAGGATGG | |
| | GGACTGAATCGATAGATCCTAATCAACCTCTGGATTACAAAATTTG | |
| | TGAAAGATTGACTGGTATTCTTAACTATGTTGCTCCTTTTACGCTAT | |
| | GTGGATACGCTGCTTTAATGCCTTTGTATCATGCTATTGCTTCCCGT | |
| | ATGGCTTTCATTTTCTCCTCCTTGTATAAATCCTGGTTGCTGTCTCT | |
| | TTATGAGGAGTTGTGGCCCGTTGTCAGGCAACGTGGCGTGGTGTGC | |
| | ACTGTGTTTGCTGACGCAACCCCCACTGGTTGGGGCATTGCCACCA | |
| | CCTGTCAGCTCCTTTCCGGGACTTTCGCTTTCCCCCTCCCTATTGCC | |
| | ACGGCGGAACTCATCGCCGCCTGCCTTGCCCGCTGCTGGACAGGG | |
| | GCTCGGCTGTTGGGCACTGACAATTCCGTGGTGTTGTCGGGGAAAT | |
| | CATCGTCCTTTCCTTGGCTGCTCGCCTGTGTTGCCACCTGGATTCTG | |
| | CGCGGGACGTCCTTCTGCTACGTCCCTTCGGCCCTCAATCCAGCGG | |
| | ACCTTCCTTCCCGCGGCCTGCTGCCGGCTCTGCGGCCTCTTCCGCGT | |
| | CTTCGCCTTCGCCCTCAGACGAGTCGGATCTCCCTTTGGGCCGCCT | |
| | CCCCGCCTGGTACCTTTAAGACCAATGACTTACAAGGCAGCTGTAG | |
| | ATCTTAGCCACTTTTTAAAAGAAAAGGGGGGACTGGAAGGGCTAA | |
| | TTCACTCCCAACGAAGACAAGATCACCTGCAGGACAGGCGCGCCC | |
| | TGCTTTTTGCTTGTACTGGGTCTCTCTGGTTAGACCAGATCTGAGCC | |
| | TGGGAGCTCTCTGGCTAACTAGGGAACCCACTGCTTAAGCCTCAAT | |
| | AAAGCTTGCCTTGAGTGCTTCAAGTAGTGTGTGCCCGTCTGTTGTG | |
| | TGACTCTGGTAACTAGAGATCCCTCAGACCCTTTTAGTCAGTGTGG | |
| | AAAATCTCTAGCACCCGGGCGATTAAGGAAAGGGCTAGATCATTC | |
| | TTGAAGACGAAAGGGCCTCGTGATACGCCTATTTTTATAGGTTAAT | |
| | GTCATGATAATAATGGTTTCTTAGACGTCAGGTGGCACTTTTCGGG | |
| | GAAATGTGCGCGGAACCCCTATTTGTTTATTTTTCTAAATACATTC | |
| | AAATATGTATCCGCTCATGAGACAATAACCCTGATAAATGCTTCAA | |
| | TAATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTGTTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGCGGCCTTTTTACGGTTCCTGGCCTTTTGCTGG | |
| | CCTTTTGCTCACATGTTCTTTCCTGCGTTATCCCCTGATTCTGTGGA | |
| | TAACCGTATTACCGCCTTTGAGTGAGCTGATACCGCTCGCCGCAGC | |
| | CGAACGACCGAGCGCAGCGAGTCAGTGAGCGAGGAAGCGGAAGA | |
| | GCGCCCAATACGCAAACCGCCTCTCCCCGCGCGTTGGCCGATTCAT | |
| | TAATGCAGCAAGCTCATGGCTGACTAATTTTTTTTATTTATGCAGA | |
| | GGCCGAGGCCGCCTCGGCCTCTGAGCTATTCCAGAAGTAGTGAGG | |
| | AGGCTTTTTTGGAGGCCTAGGCTTTTGCAAAAAGCTCCCCGTGGCA | |
| | CGACAGGTTTCCCGACTGGAAAGCGGGCAGTGAGCGCAACGCAAT | |
| | TAATGTGAGTTAGCTCACTCATTAGGCACCCCAGGCTTTACACTTT | |
| | ATGCTTCCGGCTCGTATGTTGTGTGGAATTGTGAGCGGATAACAAT | |
| | TTCACACAGGAAACAGCTATGACATGATTACGAATTTCACAAATA | |
| | AAGCATTTTTTTCACTGCATTCTAGTTGTGGTTTGTCCAAACTCATC | |
| | AATGTATCTTATCATGTCTGGATCAACTGGATAACTCAAGCTAACC | |
| | AAAATCATCCCAAACTTCCCACCCCATACCCTATTACCACTGCCAA | |
| | TTACCTGTGGTTTCATTTACTCTAAACCTGTGATTCCTCTGAATTAT | |
| | TTTCATTTTAAAGAAATTGTATTTGTTAAATATGTACTACAAACTTA | |
| | GTAGT | |
| 51 | TGGAAGGGCTAATTCACTCCCAAAGAAGACAAGATATCCTTGATC | pMA2 N-term |
| | TGTGGATCTACCACACACAAGGCTACTTCCCTGATTAGCAGAACTA | GAG-T2A-Cre |
| | CACACCAGGGCCAGGGGTCAGATATCCACTGACCTTTGGATGGTG | (no promoter) |
| | CTACAAGCTAGTACCAGTTGAGCCAGATAAGGTAGAAGAGGCCAA | |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGGTGAGTACGCCAAAAATTTTGACTAGCGG | |
| | AGGCTAGAAGGAGAGAGATGGGTGCGAGAGCGTCGGTATTAAGC | |
| | GGGGGAGAATTAGATAAATGGGAAAAAATTCGGTTAAGGCCAGG | |
| | GGGAAAGAAACAATATAAACTAAAACATATAGTATGGGCAAGCA | |
| | GGGAGCTAGAACGATTCGCAGTTAATCCTGGCCTTTTAGAGACATC | |
| | AGAAGGCTGTAGACAAATACTGGGACAGCTACAACCATCCCTTCA | |
| | GACAGGATCAGAAGAACTTAGATCATTATATAATACAATAGCAGT | |
| | CCTCTATTGTGTGCATCAAAGGATAGATGTAAAAGACACCAAGGA | |
| | AGCCTTAGATAAGATAGAGGAAGAGCAAAACAAAAGTAAGAAAA | |
| | AGGCACAGCAAGCAGGTAGTGGCGAGGGCAGAGGAAGTCTTCTAA | |
| | CATGCGGTGACGTGGAGGAGAATCCCGGCCCTATGGTGCCCAAGA | |
| | AGAAGAGGAAAGTCTCCAACCTGCTGACTGTGCACCAAAACCTGC | |
| | CTGCCCTCCCTGTGGATGCCACCTCTGATGAAGTCAGGAAGAACCT | |
| | GATGGACATGTTCAGGGACAGGCAGGCCTTCTCTGAACACACCTG | |
| | GAAGATGCTCCTGTCTGTGTGCAGATCCTGGGCTGCCTGGTGCAAG | |
| | CTGAACAACAGGAAATGGTTCCCTGCTGAACCTGAGGATGTGAGG | |
| | GACTACCTCCTGTACCTGCAAGCCAGAGGCCTGGCTGTGAAGACC | |
| | ATCCAACAGCACCTGGGCCAGCTCAACATGCTGCACAGGAGATCT | |
| | GGCCTGCCTCGCCCTTCTGACTCCAATGCTGTGTCCCTGGTGATGA | |
| | GGAGAATCAGAAAGGAGAATGTGGATGCTGGGGAGAGAGCCAAG | |
| | CAGGCCCTGGCCTTTGAACGCACTGACTTTGACCAAGTCAGATCCC | |
| | TGATGGAGAACTCTGACAGATGCCAGGACATCAGGAACCTGGCCT | |
| | TCCTGGGCATTGCCTACAACACCCTGCTGCGCATTGCCGAAATTGC | |
| | CAGAATCAGAGTGAAGGACATCTCCCGCACCGATGGTGGGAGAAT | |
| | GCTGATCCACATTGGCAGGACCAAGACCCTGGTGTCCACAGCTGG | |
| | TGTGGAGAAGGCCCTGTCCCTGGGGGTTACCAAGCTGGTGGAGAG | |
| | ATGGATCTCTGTGTCTGGTGTGGCTGATGACCCCAACAACTACCTG | |
| | TTCTGCCGGGTCAGAAAGAATGGTGTGGCTGCCCCTTCTGCCACCT | |
| | CCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCTAATCAACCTCTGGATTACAAAATTTGTGAAAGATTGAC | |
| | TGGTATTCTTAACTATGTTGCTCCTTTTACGCTATGTGGATACGCTG | |
| | CTTTAATGCCTTTGTATCATGCTATTGCTTCCCGTATGGCTTTCATT | |
| | TTCTCCTCCTTGTATAAATCCTGGTTGCTGTCTCTTTATGAGGAGTT | |
| | GTGGCCCGTTGTCAGGCAACGTGGCGTGGTGTGCACTGTGTTTGCT | |
| | GACGCAACCCCCACTGGTTGGGGCATTGCCACCACCTGTCAGCTCC | |
| | TTTCCGGGACTTTCGCTTTCCCCCTCCCTATTGCCACGGCGGAACTC | |
| | ATCGCCGCCTGCCTTGCCCGCTGCTGGACAGGGGCTCGGCTGTTGG | |
| | GCACTGACAATTCCGTGGTGTTGTCGGGGAAATCATCGTCCTTTCC | |
| | TTGGCTGCTCGCCTGTGTTGCCACCTGGATTCTGCGCGGGACGTCC | |
| | TTCTGCTACGTCCCTTCGGCCCTCAATCCAGCGGACCTTCCTTCCCG | |
| | CGGCCTGCTGCCGGCTCTGCGGCCTCTTCCGCGTCTTCGCCTTCGCC | |
| | CTCAGACGAGTCGGATCTCCCTTTGGGCCGCCTCCCCGCCTGAGAT | |
| | CCTTTAAGACCAATGACTTACAAGGCAGCTGTAGATCTTAGCCACT | |
| | TTTTAAAAGAAAAGGGGGGACTGGAAGGGCTAATTCACTCCCAAC | |
| | GAAGACAAGATCTGCTTTTTGCTTGTACTGGGTCTCTCTGGTTAGA | |
| | CCAGATCTGAGCCTGGGAGCTCTCTGGCTAACTAGGGAACCCACT | |
| | GCTTAAGCCTCAATAAAGCTTGCCTTGAGTGCTTCAAGTAGTGTGT | |
| | GCCCGTCTGTTGTGTGACTCTGGTAACTAGAGATCCCTCAGACCCT | |
| | TTTAGTCAGTGTGGAAAATCTCTAGCAGTAGTAGTTCATGTCATCT | |
| | TATTATTCAGTATTTATAACTTGCAAAGAAATGAATATCAGAGAGT | |
| | GAGAGGCCCGGGTTAATTAAGGAAAGGGCTAGATCATTCTTGAAG | |
| | ACGAAAGGGCCTCGTGATACGCCTATTTTTATAGGTTAATGTCATG | |
| | ATAATAATGGTTTCTTAGACGTCAGGTGGCACTTTTCGGGGAAATG | |
| | TGCGCGGAACCCCTATTTGTTTATTTTTCTAAATACATTCAAATATG | |
| | TATCCGCTCATGAGACAATAACCCTGATAAATGCTTCAATAATATT | |
| | GAAAAAGGAAGAGTATGAGTATTCAACATTTCCGTGTCGCCCTTAT | |
| | TCCCTTTTTTGCGGCATTTTGCCTTCCTGTTTTTGCTCACCCAGAAA | |
| | CGCTGGTGAAAGTAAAAGATGCTGAAGATCAGTTGGGTGCACGAG | |
| | TGGGTTACATCGAACTGGATCTCAACAGCGGTAAGATCCTTGAGA | |
| | GTTTTCGCCCCGAAGAACGTTTTCCAATGATGAGCACTTTTAAAGT | |
| | TCTGCTATGTGGCGCGGTATTATCCCGTGTTGACGCCGGGCAAGAG | |
| | CAACTCGGTCGCCGCATACACTATTCTCAGAATGACTTGGTTGAGT | |
| | ACTCACCAGTCACAGAAAAGCATCTTACGGATGGCATGACAGTAA | |
| | GAGAATTATGCAGTGCTGCCATAACCATGAGTGATAACACTGCGG | |
| | CCAACTTACTTCTGACAACGATCGGAGGACCGAAGGAGCTAACCG | |
| | CTTTTTTGCACAACATGGGGGATCATGTAACTCGCCTTGATCGTTG | |
| | GGAACCGGAGCTGAATGAAGCCATACCAAACGACGAGCGTGACAC | |
| | CACGATGCCTGTAGCAATGGCAACAACGTTGCGCAAACTATTAAC | |
| | TGGCGAACTACTTACTCTAGCTTCCCGGCAACAATTAATAGACTGG | |
| | ATGGAGGCGGATAAAGTTGCAGGACCACTTCTGCGCTCGGCCCTTC | |
| | CGGCTGGCTGGTTTATTGCTGATAAATCTGGAGCCGGTGAGCGTGG | |
| | GTCTCGCGGTATCATTGCAGCACTGGGGCCAGATGGTAAGCCCTCC | |
| | CGTATCGTAGTTATCTACACGACGGGGAGTCAGGCAACTATGGAT | |
| | GAACGAAATAGACAGATCGCTGAGATAGGTGCCTCACTGATTAAG | |
| | CATTGGTAACTGTCAGACCAAGTTTACTCATATATACTTTAGATTG | |
| | ATTTAAAACTTCATTTTTAATTTAAAAGGATCTAGGTGAAGATCCT | |
| | TTTTGATAATCTCATGACCAAAATCCCTTAACGTGAGTTTTCGTTCC | |
| | ACTGAGCGTCAGACCCCGTAGAAAAGATCAAAGGATCTTCTTGAG | |
| | ATCCTTTTTTTCTGCGCGTAATCTGCTGCTTGCAAACAAAAAAACC | |
| | ACCGCTACCAGCGGTGGTTTGTTTGCCGGATCAAGAGCTACCAACT | |
| | CTTTTTCCGAAGGTAACTGGCTTCAGCAGAGCGCAGATACCAAATA | |
| | CTGTTCTTCTAGTGTAGCCGTAGTTAGGCCACCACTTCAAGAACTC | |
| | TGTAGCACCGCCTACATACCTCGCTCTGCTAATCCTGTTACCAGTG | |
| | GCTGCTGCCAGTGGCGATAAGTCGTGTCTTACCGGGTTGGACTCAA | |
| | GACGATAGTTACCGGATAAGGCGCAGCGGTCGGGCTGAACGGGGG | |
| | GTTCGTGCACACAGCCCAGCTTGGAGCGAACGACCTACACCGAAC | |
| | TGAGATACCTACAGCGTGAGCTATGAGAAAGCGCCACGCTTCCCG | |
| | AAGGGAGAAAGGCGGACAGGTATCCGGTAAGCGGCAGGGTCGGA | |
| | ACAGGAGAGCGCACGAGGGAGCTTCCAGGGGGAAACGCCTGGTAT | |
| | CTTTATAGTCCTGTCGGGTTTCGCCACCTCTGACTTGAGCGTCGATT | |
| | TTTGTGATGCTCGTCAGGGGGGCGGAGCCTATGGAAAAACGCCAG | |
| | CAACGCGGCCTTTTTACGGTTCCTGGCCTTTTGCTGGCCTTTTGCTC | |
| | ACATGTTCTTTCCTGCGTTATCCCCTGATTCTGTGGATAACCGTATT | |
| | ACCGCCTTTGAGTGAGCTGATACCGCTCGCCGCAGCCGAACGACC | |
| | GAGCGCAGCGAGTCAGTGAGCGAGGAAGCGGAAGAGCGCCCAAT | |
| | ACGCAAACCGCCTCTCCCCGCGCGTTGGCCGATTCATTAATGCAGC | |
| | AAGCTCATGGCTGACTAATTTTTTTTATTTATGCAGAGGCCGAGGC | |
| | CGCCTCGGCCTCTGAGCTATTCCAGAAGTAGTGAGGAGGCTTTTTT | |
| | GGAGGCCTAGGCTTTTGCAAAAAGCTCCCCGTGGCACGACAGGTT | |
| | TCCCGACTGGAAAGCGGGCAGTGAGCGCAACGCAATTAATGTGAG | |
| | TTAGCTCACTCATTAGGCACCCCAGGCTTTACACTTTATGCTTCCG | |
| | GCTCGTATGTTGTGTGGAATTGTGAGCGGATAACAATTTCACACAG | |
| | GAAACAGCTATGACATGATTACGAATTTCACAAATAAAGCATTTTT | |
| | TTCACTGCATTCTAGTTGTGGTTTGTCCAAACTCATCAATGTATCTT | |
| | ATCATGTCTGGATCAACTGGATAACTCAAGCTAACCAAAATCATCC | |
| | CAAACTTCCCACCCCATACCCTATTACCACTGCCAATTACCTGTGG | |
| | TTTCATTTACTCTAAACCTGTGATTCCTCTGAATTATTTTCATTTTA | |
| | AAGAAATTGTATTTGTTAAATATGTACTACAAACTTAGTAGT | |
| 52 | GARASVLSGGELDKWEKIRLRPGGKKQYKLKHIVWASRELERFAVNP | N-term gag |
| | GLLETSEGCRQILGQLQPSLQTGSEELRSLYNTIAVLYCVHQRIDVKDT | (no Met) |
| | KEALDKIEEEQNKSKKKAQQA | |
| 53 | AGCTTGGCCCATTGCATACGTTGTATCCATATCATAATATGTACAT | MA-MS2_{cp}- |
| | TTATATTGGCTCATGTCCAACATTACCGCCATGTTGACATTGATTAT | CA gagpol |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCTAGACTGCCATGGGCGCCCGCGCCTCCGTG | |
| | CTGTCCGGCGGCGAGCTGGACAAGTGGGAGAAGATCCGCCTGCGC | |
| | CCCGGCGGCAAGAAGCAGTACAAGCTGAAGCACATCGTGTGGGCC | |
| | TCCCGCGAGCTGGAGCGCTTCGCCGTGAACCCCGGCCTGCTGGAG | |
| | ACCTCCGAGGGCTGCCGCCAGATCCTGGGCCAGCTGCAGCCCTCCC | |
| | TGCAAACCGGCTCCGAGGAGCTGCGCTCCCTGTACAACACCATCG | |
| | CCGTGCTGTACTGCGTGCACCAGCGCATCGACGTGAAGGACACCA | |
| | AGGAGGCCCTGGACAAGATCGAGGAGGAGCAGAACAAGTCCAAG | |
| | AAGAAGGCCCAGCAGGCCGCCGCCGACACCGGCAACAACTCCCAG | |
| | GTGTCCCAGAACTACCCCATCGTGCAGATGGCCAGCAATTTTACGC | |
| | AATTCGTGCTCGTGGACAACGGCGGCACGGGCGACGTGACCGTGG | |
| | CCCCCAGCAACTTCGCCAATGGCATCGCCGAATGGATCAGCAGCA | |
| | ACAGCAGGAGCCAGGCGTATAAAGTTACGTGCAGCGTCAGACAGA | |
| | GCAGCGCCCAGAACAGGAAATATACGATCAAGGTCGAGGTTCCCA | |
| | AGGGAGCTTGGAGGAGCTATCTTAATATGGAGCTGACCATCCCCA | |
| | TCTTCGCGACAAATTCAGACTGCGAGCTCATCGTGAAGGCAATGC | |
| | AGGGCCTCTTGAAAGATGGCAACCCCATCCCAAGCGCAATCGCGG | |
| | CCAACTCAGGAATCTACTCCCAGAACTACCCCATCGTGCAGAACCT | |
| | GCAGGGCCAGATGGTGCACCAGGCCATCTCCCCCCGCACCCTGAA | |
| | CGCCTGGGTGAAGGTGGTGGAGGAGAAGGCCTTCTCCCCCGAAGT | |
| | CATCCCCATGTTCTCCGCCCTGTCCGAGGGCGCCACCCCCCAGGAC | |
| | CTGAACACCATGCTGAACACCGTGGGCGGCCACCAGGCCGCCATG | |
| | CAGATGCTGAAGGAGACCATCAACGAGGAGGCCGCCGAGTGGGA | |
| | CCGCCTGCACCCCGTGCACGCCGGCCCCATCGCCCCCGGCCAGATG | |
| | CGCGAGCCCCGCGGCTCCGACATCGCCGGCACCACCTCCACCCTGC | |
| | AAGAGCAGATCGGCTGGATGACCCACAACCCCCCCATCCCCGTGG | |
| | GCGAGATCTACAAGCGCTGGATCATCCTGGGCCTGAACAAGATCG | |
| | TGCGCATGTACTCCCCCACCTCCATCCTGGACATCCGCCAGGGCCC | |
| | CAAGGAGCCCTTCCGCGACTACGTGGACCGCTTCTACAAGACCCTG | |
| | CGCGCCGAGCAGGCCTCCCAGGAGGTAAAGAACTGGATGACCGAG | |
| | ACCCTGCTGGTGCAGAACGCCAACCCCGACTGCAAGACCATCCTG | |
| | AAGGCCCTGGGCCCCGGCGCCACCCTGGAGGAGATGATGACCGCC | |
| | TGCCAGGGCGTGGGCGGCCCCGGCCACAAGGCCCGCGTGCTGGCC | |
| | GAGGCCATGTCCCAAGTCACCAACCCCGCCACCATCATGATCCAG | |
| | AAGGGCAACTTCCGCAACCAGCGCAAGACCGTGAAGTGCTTCAAC | |
| | TGCGGCAAGGAGGGCCACATCGCCAAGAACTGCCGCGCCCCCCGC | |
| | AAGAAGGGCTGCTGGAAGTGCGGCAAGGAGGGCCACCAGATGAA | |
| | AGATTGTACTGAGAGACAGGCTAATTTTTTAGGGAAGATCTGGCCT | |
| | TCCCACAAGGGAAGGCCAGGGAATTTTCTTCAGAGCAGACCAGAG | |
| | CCAACAGCCCCACCAGAAGAGAGCTTCAGGTTTGGGGAAGAGACA | |
| | ACAACTCCCTCTCAGAAGCAGGAGCCGATAGACAAGGAACTGTAT | |
| | CCTTTAGCTTCCCTCAGATCACTCTTTGGCAGCGACCCCTCGTCAC | |
| | AATAAAGATCGGTGGCCAGCTGAAGGAGGCCCTGCTGGACACCGG | |
| | CGCCGACGACACCGTGCTGGAGGAGATGAACCTGCCCGGCCGCTG | |
| | GAAGCCCAAGATGATCGGCGGCATCGGCGGCTTCATCAAAGTCCG | |
| | CCAGTACGACCAGATCCTGATCGAGATCTGCGGCCACAAGGCCAT | |
| | CGGCACCGTGCTGGTGGGCCCCACCCCCGTGAACATCATCGGCCG | |
| | CAACCTGCTGACCCAGATCGGCTGCACCCTGAACTTCCCCATCTCC | |
| | CCCATCGAGACCGTGCCCGTGAAGCTGAAGCCCGGCATGGACGGC | |
| | CCCAAAGTCAAGCAGTGGCCCCTGACCGAGGAGAAGATCAAGGCC | |
| | CTGGTGGAGATCTGCACCGAGATGGAGAAGGAGGGCAAGATCTCC | |
| | AAGATCGGCCCCGAGAACCCCTACAACACCCCCGTGTTCGCCATC | |
| | AAGAAGAAGGACTCCACCAAGTGGCGCAAGCTGGTGGACTTCCGC | |
| | GAGCTGAACAAGCGCACCCAGGACTTCTGGGAGGTGCAGCTGGGC | |
| | ATCCCCCACCCCGCCGGCCTGAAGCAGAAGAAGTCCGTGACCGTG | |
| | CTGGACGTGGGCGACGCCTACTTCTCCGTGCCCCTGGACAAGGACT | |
| | TCCGCAAGTACACCGCCTTCACCATCCCCTCCATCAACAACGAGAC | |
| | CCCCGGCATCCGCTACCAGTACAACGTGCTGCCCCAGGGCTGGAA | |
| | GGGCTCCCCCGCCATCTTCCAGTGCTCCATGACCAAGATCCTGGAG | |
| | CCCTTCCGCAAGCAGAACCCCGACATCGTGATCTACCAGTACATGG | |
| | ACGACCTGTACGTGGGCTCCGACCTGGAGATCGGCCAGCACCGCA | |
| | CCAAGATCGAGGAGCTGCGCCAGCACCTGCTGCGCTGGGGCTTCA | |
| | CCACCCCCGACAAGAAGCACCAGAAGGAGCCCCCCTTCCTGTGGA | |
| | TGGGCTACGAGCTGCACCCCGACAAGTGGACCGTGCAGCCCATCG | |
| | TGCTGCCCGAGAAGGACTCCTGGACCGTGAACGACATCCAGAAGC | |
| | TGGTGGGCAAGCTGAACTGGGCCTCCCAGATCTACGCCGGCATCA | |
| | AAGTCCGCCAGCTGTGCAAGCTGCTGCGCGGCACCAAGGCCCTGA | |
| | CCGAGGTGGTGCCCCTGACCGAGGAGGCCGAGCTGGAGCTGGCCG | |
| | AGAACCGCGAGATCCTGAAGGAGCCCGTGCACGGCGTGTACTACG | |
| | ACCCCTCCAAGGACCTGATCGCCGAGATCCAGAAGCAGGGCCAGG | |
| | GCCAGTGGACCTACCAGATCTACCAGGAGCCCTTCAAGAACCTGA | |
| | AGACCGGCAAATACGCCCGCATGAAGGGCGCCCACACCAACGACG | |
| | TGAAGCAGCTGACCGAGGCCGTGCAGAAGATCGCCACCGAGTCCA | |
| | TCGTGATCTGGGGCAAGACTCCCAAGTTCAAGCTGCCCATCCAGA | |
| | AGGAGACCTGGGAGGCCTGGTGGACCGAGTACTGGCAGGCCACCT | |
| | GGATCCCCGAGTGGGAGTTCGTGAACACCCCCCCCCTGGTGAAGC | |
| | TGTGGTACCAGCTGGAGAAGGAGCCCATCATCGGCGCCGAGACCT | |
| | TCTACGTGGACGGCGCCGCCAACCGCGAGACCAAGCTGGGCAAGG | |
| | CCGGCTACGTGACCGACCGCGGCCGCCAGAAGGTGGTGCCCCTGA | |
| | CCGACACCACCAACCAGAAGACCGAGCTGCAGGCCATCCACCTGG | |
| | CCCTGCAAGACTCCGGCCTGGAGGTGAACATCGTGACCGACTCCC | |
| | AGTATGCATTGGGCATCATCCAGGCCCAGCCCGACAAGTCCGAGT | |
| | CCGAGCTGGTGTCCCAGATCATCGAGCAGCTGATCAAGAAGGAGA | |
| | AGGTGTACCTGGCCTGGGTGCCCGCCCACAAGGGCATCGGCGGCA | |
| | ACGAGCAGGTGGACAAGCTGGTGTCCGCCGGCATCCGCAAGGTGC | |
| | TGTTCCTGGACGGCATCGACAAGGCCCAGGAGGAGCACGAGAAGT | |
| | ACCACTCCAACTGGCGCGCCATGGCCTCCGACTTCAACCTGCCCCC | |
| | CGTGGTGGCCAAGGAGATCGTGGCCTCCTGCGACAAGTGCCAGCT | |
| | GAAGGGCGAGGCCATGCACGGCCAGGTGGACTGCTCCCCCGGCAT | |
| | CTGGCAGCTGGACTGCACCCACCTGGAGGGCAAGGTGATCCTGGT | |
| | GGCCGTGCACGTGGCCTCCGGCTACATCGAGGCCGAGGTGATCCC | |
| | CGCCGAGACCGGCCAGGAGACCGCCTACTTCCTGCTGAAGCTGGC | |
| | CGGCCGCTGGCCCGTGAAGACCGTGCACACCGACAACGGCTCCAA | |
| | CTTCACCTCCACCACCGTGAAGGCCGCCTGCTGGTGGGCCGGCATC | |
| | AAGCAGGAGTTCGGCATCCCCTACAACCCCCAGTCCCAGGGCGTG | |
| | ATCGAGTCCATGAACAAGGAGCTGAAGAAGATCATCGGCCAAGTC | |
| | CGCGACCAGGCCGAGCACCTGAAGACCGCCGTGCAGATGGCCGTG | |
| | TTCATCCACAACTTCAAGCGCAAGGGCGGCATCGGCGGCTACTCC | |
| | GCCGGCGAGCGCATCGTGGACATCATCGCCACCGACATCCAGACC | |
| | AAGGAGCTGCAGAAGCAGATCACCAAGATCCAGAACTTCCGCGTG | |
| | TACTACCGCGACTCCCGCGACCCCGTGTGGAAGGGCCCCGCCAAG | |
| | CTGCTGTGGAAGGGCGAGGGCGCCGTGGTGATCCAGGACAACTCC | |
| | GACATCAAGGTGGTGCCCCGCCGCAAGGCCAAGATCATCCGCGAC | |
| | TACGGCAAGCAGATGGCCGGCGACGACTGCGTGGCCTCCCGCCAG | |
| | GACGAGGACTAACACATGGAAAAGATTAGTAAAACACCATAGGCC | |
| | GCTCTAGAGGATCCAAGCTTATCGATACCGTCGACCTCGAGGGCCC | |
| | AGATCTAATTCACCCCACCAGTGCAGGCTGCCTATCAGAAAGTGGT | |
| | GGCTGGTGTGGCTAATGCCCTGGCCCACAAGTATCACTAAGCTCGC | |
| | TTTCTTGCTGTCCAATTTCTATTAAAGGTTCCTTTGTTCCCTAAGTC | |
| | CAACTACTAAACTGGGGGATATTATGAAGGGCCTTGAGCATCTGG | |
| | ATTCTGCCTAATAAAAAACATTTATTTTCATTGCAATGATGTATTTA | |
| | AATTATTTCTGAATATTTTACTAAAAAGGGAATGTGGGAGGTCAGT | |
| | GCATTTAAAACATAAAGAAATGAAGAGCTAGTTCAAACCTTGGGA | |
| | AAATACACTATATCTTAAACTCCATGAAAGAAGGTGAGGCTGCAA | |
| | ACAGCTAATGCACATTGGCAACAGCCCCTGATGCCTATGCCTTATT | |
| | CATCCCTCAGAAAAGGATTCAAGTAGAGGCTTGATTTGGAGGTTA | |
| | AAGTTTTGCTATGCTGTATTTTACATTACTTATTGTTTTAGCTGTCC | |
| | TCATGAATGTCTTTTCACTACCCATTTGCTTATCCTGCATCTCTCAG | |
| | CCTTGACTCCACTCAGTTCTCTTGCTTAGAGATACCACCTTTCCCCT | |
| | GAAGTGTTCCTTCCATGTTTTACGGCGAGATGGTTTCTCCTCGCCTG | |
| | GCCACTCAGCCTTAGTTGTCTCTGTTGTCTTATAGAGGTCTACTTGA | |
| | AGAAGGAAAAACAGGGGGCATGGTTTGACTGTCCTGTGAGCCCTT | |
| | CTTCCCTGCCTCCCCCACTCACAGTGACCCGGAATCCCTCGACATG | |
| | GCAGTCTAGATCATTCTTGAAGACGAAAGGGCCTCGTGATACGCCT | |
| | ATTTTTATAGGTTAATGTCATGATAATAATGGTTTCTTAGACGTCA | |
| | GGTGGCACTTTTCGGGGAAATGTGCGCGGAACCCCTATTTGTTTAT | |
| | TTTTCTAAATACATTCAAATATGTATCCGCTCATGAGACAATAACC | |
| | CTGATAAATGCTTCAATAATATTGAAAAAGGAAGAGTATGAGTAT | |
| | TCAACATTTCCGTGTCGCCCTTATTCCCTTTTTTGCGGCATTTTGCC | |
| | TTCCTGTTTTTGCTCACCCAGAAACGCTGGTGAAAGTAAAAGATGC | |
| | TGAAGATCAGTTGGGTGCACGAGTGGGTTACATCGAACTGGATCT | |
| | CAACAGCGGTAAGATCCTTGAGAGTTTTCGCCCCGAAGAACGTTTT | |
| | CCAATGATGAGCACTTTTAAAGTTCTGCTATGTGGCGCGGTATTAT | |
| | CCCGTATTGACGCCGGGCAAGAGCAACTCGGTCGCCGCATACACT | |
| | ATTCTCAGAATGACTTGGTTGAGTACTCACCAGTCACAGAAAAGC | |
| | ATCTTACGGATGGCATGACAGTAAGAGAATTATGCAGTGCTGCCA | |
| | TAACCATGAGTGATAACACTGCGGCCAACTTACTTCTGACAACGAT | |
| | CGGAGGACCGAAGGAGCTAACCGCTTTTTTGCACAACATGGGGGA | |
| | TCATGTAACTCGCCTTGATCGTTGGGAACCGGAGCTGAATGAAGCC | |
| | ATACCAAACGACGAGCGTGACACCACGATGCCTGTAGCAATGGCA | |
| | ACAACGTTGCGCAAACTATTAACTGGCGAACTACTTACTCTAGCTT | |
| | CCCGGCAACAATTAATAGACTGGATGGAGGCGGATAAAGTTGCAG | |
| | GACCACTTCTGCGCTCGGCCCTTCCGGCTGGCTGGTTTATTGCTGA | |
| | TAAATCTGGAGCCGGTGAGCGTGGGTCTCGCGGTATCATTGCAGC | |
| | ACTGGGGCCAGATGGTAAGCCCTCCCGTATCGTAGTTATCTACACG | |
| | ACGGGGAGTCAGGCAACTATGGATGAACGAAATAGACAGATCGCT | |
| | GAGATAGGTGCCTCACTGATTAAGCATTGGTAACTGTCAGACCAA | |
| | GTTTACTCATATATACTTTAGATTGATTTAAAACTTCATTTTTAATT | |
| | TAAAAGGATCTAGGTGAAGATCCTTTTTGATAATCTCATGACCAAA | |
| | ATCCCTTAACGTGAGTTTTCGTTCCACTGAGCGTCAGACCCCGTAG | |
| | AAAAGATCAAAGGATCTTCTTGAGATCCTTTTTTTCTGCGCGTAAT | |
| | CTGCTGCTTGCAAACAAAAAAACCACCGCTACCAGCGGTGGTTTGT | |
| | TTGCCGGATCAAGAGCTACCAACTCTTTTTCCGAAGGTAACTGGCT | |
| | TCAGCAGAGCGCAGATACCAAATACTGTTCTTCTAGTGTAGCCGTA | |
| | GTTAGGCCACCACTTCAAGAACTCTGTAGCACCGCCTACATACCTC | |
| | GCTCTGCTAATCCTGTTACCAGTGGCTGCTGCCAGTGGCGATAAGT | |
| | CGTGTCTTACCGGGTTGGACTCAAGACGATAGTTACCGGATAAGG | |
| | CGCAGCGGTCGGGCTGAACGGGGGGTTCGTGCACACAGCCCAGCT | |
| | TGGAGCGAACGACCTACACCGAACTGAGATACCTACAGCGTGAGC | |
| | TATGAGAAAGCGCCACGCTTCCCGAAGGGAGAAAGGCGGACAGGT | |
| | ATCCGGTAAGCGGCAGGGTCGGAACAGGAGAGCGCACGAGGGAG | |
| | CTTCCAGGGGGAAACGCCTGGTATCTTTATAGTCCTGTCGGGTTTC | |
| | GCCACCTCTGACTTGAGCGTCGATTTTTGTGATGCTCGTCAGGGGG | |
| | GCGGAGCCTATGGAAAAACGCCAGCAACGGAGATGCGCCGCGTGC | |
| | GGCTGCTGGAGATGGCGGACGCGATGGATATGTTCTGCCAAGGGT | |
| | TGGTTTGCGCATTCACAGTTCTCCGCAAGAATTGATTGGCTCCAAT | |
| | TCTTGGAGTGGTGAATCCGTTAGCGAGGTGCCGCCGGCTTCCATTC | |
| | AGGTCGAGGTGGCCCGGCTCCATGCACCGCGACGCAACGCGGGGA | |
| | GGCAGACAAGGTATAGGGCGGCGCCTACAATCCATGCCAACCCGT | |
| | TCCATGTGCTCGCCGAGGCGGCATAAATCGCCGTGACGATCAGCG | |
| | GTCCAATGATCGAAGTTAGGCTGGTAAGAGCCGCGAGCGATCCTT | |
| | GAAGCTGTCCCTGATGGTCGTCATCTACCTGCCTGGACAGCATGGC | |
| | CTGCAACGCGGGCATCCCGATGCCGCCGGAAGCGAGAAGAATCAT | |
| | AATGGGGAAGGCCATCCAGCCTCGCGTCGGGGAGCTTTTTGCAAA | |
| | AGCCTAGGCCTCCAAAAAAGCCTCCTCACTACTTCTGGAATAGCTC | |
| | AGAGGCCGAGGCGGCCTCGGCCTCTGCATAAATAAAAAAAATTAG | |
| | TCAGCCATG | |
| 54 | AGCTTGGCCCATTGCATACGTTGTATCCATATCATAATATGTACAT | N-term PH |
| | TTATATTGGCTCATGTCCAACATTACCGCCATGTTGACATTGATTAT | domain |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGACAGCGGG | |
| | AGGGATTTCCTCACGCTGCACGGTTTGCAGGACGACGAGGACCTC | |
| | CAAGCTTTGCTCAAAGGCAGTCAACTTCTCAAGGTCAAGAGCAGC | |
| | TCCTGGAGGCGCGAACGGTTTTACAAGTTGCAAGAAGATTGCAAA | |
| | ACAATTTGGCAAGAGAGCCGGAAAGTCATGAGAACTCCCGAGAGC | |
| | CAACTGTTCAGCATCGAGGACATCCAAGAAGTCAGGATGGGCCAT | |
| | AGGACCGAGGGCTTGGAAAAATTCGCCAGGGACGTGCCCGAAGAC | |
| | CGATGCTTTAGCATCGTGTTCAAAGATCAGAGGAACACGTTGGACT | |
| | TGATCGCCCCCAGTCCGGCTGATGCCCAGCATTGGGTTCTGGGGCT | |
| | CCATAAGATCATCCATCATAGCGGCAGCATGGACCAGAGGCAGAA | |
| | ACTCCAACATTGGATTCATAGTTGTCTTAGGAAAGCCGACAAGAA | |
| | CAAGGACAACAAGATGAGCTTCAAGGAGTTGCAGAATTTTCTTAA | |
| | AGAGCTGAATATCCAGTCCCAGAACTACCCCATCGTGCAGATGGC | |
| | CAGCAATTTTACGCAATTCGTGCTCGTGGACAACGGCGGCACGGG | |
| | CGACGTGACCGTGGCCCCCAGCAACTTCGCCAATGGCATCGCCGA | |
| | ATGGATCAGCAGCAACAGCAGGAGCCAGGCGTATAAAGTTACGTG | |
| | CAGCGTCAGACAGAGCAGCGCCCAGAACAGGAAATATACGATCAA | |
| | GGTCGAGGTTCCCAAGGGAGCTTGGAGGAGCTATCTTAATATGGA | |
| | GCTGACCATCCCCATCTTCGCGACAAATTCAGACTGCGAGCTCATC | |
| | GTGAAGGCAATGCAGGGCCTCTTGAAAGATGGCAACCCCATCCCA | |
| | AGCGCAATCGCGGCCAACTCAGGAATCTACTAAGGATCCAAGCTT | |
| | ATCGATACCGTCGACCTCGAGGGCCCAGATCTAATTCACCCCACCA | |
| | GTGCAGGCTGCCTATCAGAAAGTGGTGGCTGGTGTGGCTAATGCC | |
| | CTGGCCCACAAGTATCACTAAGCTCGCTTTCTTGCTGTCCAATTTCT | |
| | ATTAAAGGTTCCTTTGTTCCCTAAGTCCAACTACTAAACTGGGGGA | |
| | TATTATGAAGGGCCTTGAGCATCTGGATTCTGCCTAATAAAAAACA | |
| | TTTATTTTCATTGCAATGATGTATTTAAATTATTTCTGAATATTTTA | |
| | CTAAAAAGGGAATGTGGGAGGTCAGTGCATTTAAAACATAAAGAA | |
| | ATGAAGAGCTAGTTCAAACCTTGGGAAAATACACTATATCTTAAA | |
| | CTCCATGAAAGAAGGTGAGGCTGCAAACAGCTAATGCACATTGGC | |
| | AACAGCCCCTGATGCCTATGCCTTATTCATCCCTCAGAAAAGGATT | |
| | CAAGTAGAGGCTTGATTTGGAGGTTAAAGTTTTGCTATGCTGTATT | |
| | TTACATTACTTATTGTTTTAGCTGTCCTCATGAATGTCTTTTCACTA | |
| | CCCATTTGCTTATCCTGCATCTCTCAGCCTTGACTCCACTCAGTTCT | |
| | CTTGCTTAGAGATACCACCTTTCCCCTGAAGTGTTCCTTCCATGTTT | |
| | TACGGCGAGATGGTTTCTCCTCGCCTGGCCACTCAGCCTTAGTTGT | |
| | CTCTGTTGTCTTATAGAGGTCTACTTGAAGAAGGAAAAACAGGGG | |
| | GCATGGTTTGACTGTCCTGTGAGCCCTTCTTCCCTGCCTCCCCCACT | |
| | CACAGTGACCCGGAATCCCTCGACATGGCAGTCTAGATCATTCTTG | |
| | AAGACGAAAGGGCCTCGTGATACGCCTATTTTTATAGGTTAATGTC | |
| | ATGATAATAATGGTTTCTTAGACGTCAGGTGGCACTTTTCGGGGAA | |
| | ATGTGCGCGGAACCCCTATTTGTTTATTTTTCTAAATACATTCAAAT | |
| | ATGTATCCGCTCATGAGACAATAACCCTGATAAATGCTTCAATAAT | |
| | ATTGAAAAAGGAAGAGTATGAGTATTCAACATTTCCGTGTCGCCCT | |
| | TATTCCCTTTTTTGCGGCATTTTGCCTTCCTGTTTTTGCTCACCCAG | |
| | AAACGCTGGTGAAAGTAAAAGATGCTGAAGATCAGTTGGGTGCAC | |
| | GAGTGGGTTACATCGAACTGGATCTCAACAGCGGTAAGATCCTTG | |
| | AGAGTTTTCGCCCCGAAGAACGTTTTCCAATGATGAGCACTTTTAA | |
| | AGTTCTGCTATGTGGCGCGGTATTATCCCGTATTGACGCCGGGCAA | |
| | GAGCAACTCGGTCGCCGCATACACTATTCTCAGAATGACTTGGTTG | |
| | AGTACTCACCAGTCACAGAAAAGCATCTTACGGATGGCATGACAG | |
| | TAAGAGAATTATGCAGTGCTGCCATAACCATGAGTGATAACACTG | |
| | CGGCCAACTTACTTCTGACAACGATCGGAGGACCGAAGGAGCTAA | |
| | CCGCTTTTTTGCACAACATGGGGGATCATGTAACTCGCCTTGATCG | |
| | TTGGGAACCGGAGCTGAATGAAGCCATACCAAACGACGAGCGTGA | |
| | CACCACGATGCCTGTAGCAATGGCAACAACGTTGCGCAAACTATT | |
| | AACTGGCGAACTACTTACTCTAGCTTCCCGGCAACAATTAATAGAC | |
| | TGGATGGAGGCGGATAAAGTTGCAGGACCACTTCTGCGCTCGGCC | |
| | CTTCCGGCTGGCTGGTTTATTGCTGATAAATCTGGAGCCGGTGAGC | |
| | GTGGGTCTCGCGGTATCATTGCAGCACTGGGGCCAGATGGTAAGC | |
| | CCTCCCGTATCGTAGTTATCTACACGACGGGGAGTCAGGCAACTAT | |
| | GGATGAACGAAATAGACAGATCGCTGAGATAGGTGCCTCACTGAT | |
| | TAAGCATTGGTAACTGTCAGACCAAGTTTACTCATATATACTTTAG | |
| | ATTGATTTAAAACTTCATTTTTAATTTAAAAGGATCTAGGTGAAGA | |
| | TCCTTTTTGATAATCTCATGACCAAAATCCCTTAACGTGAGTTTTCG | |
| | TTCCACTGAGCGTCAGACCCCGTAGAAAAGATCAAAGGATCTTCTT | |
| | GAGATCCTTTTTTTCTGCGCGTAATCTGCTGCTTGCAAACAAAAAA | |
| | ACCACCGCTACCAGCGGTGGTTTGTTTGCCGGATCAAGAGCTACCA | |
| | ACTCTTTTTCCGAAGGTAACTGGCTTCAGCAGAGCGCAGATACCAA | |
| | ATACTGTTCTTCTAGTGTAGCCGTAGTTAGGCCACCACTTCAAGAA | |
| | CTCTGTAGCACCGCCTACATACCTCGCTCTGCTAATCCTGTTACCA | |
| | GTGGCTGCTGCCAGTGGCGATAAGTCGTGTCTTACCGGGTTGGACT | |
| | CAAGACGATAGTTACCGGATAAGGCGCAGCGGTCGGGCTGAACGG | |
| | GGGGTTCGTGCACACAGCCCAGCTTGGAGCGAACGACCTACACCG | |
| | AACTGAGATACCTACAGCGTGAGCTATGAGAAAGCGCCACGCTTC | |
| | CCGAAGGGAGAAAGGCGGACAGGTATCCGGTAAGCGGCAGGGTC | |
| | GGAACAGGAGAGCGCACGAGGGAGCTTCCAGGGGGAAACGCCTG | |
| | GTATCTTTATAGTCCTGTCGGGTTTCGCCACCTCTGACTTGAGCGTC | |
| | GATTTTTGTGATGCTCGTCAGGGGGGCGGAGCCTATGGAAAAACG | |
| | CCAGCAACGGATGCGCCGCGTGCGGCTGCTGGAGATGGCGGACGC | |
| | GATGGATATGTTCTGCCAAGGGTTGGTTTGCGCATTCACAGTTCTC | |
| | CGCAAGAATTGATTGGCTCCAATTCTTGGAGTGGTGAATCCGTTAG | |
| | CGAGGTGCCGCCGGCTTCCATTCAGGTCGAGGTGGCCCGGCTCCAT | |
| | GCACCGCGACGCAACGCGGGGAGGCAGACAAGGTATAGGGCGGC | |
| | GCCTACAATCCATGCCAACCCGTTCCATGTGCTCGCCGAGGCGGCA | |
| | TAAATCCCCGTGACGATCAGCGGTCCAATGATCGAAGTTAGGCTG | |
| | GTAAGAGCCGCGAGCGATCCTTGAAGCTGTCCCTGATGGTCGTCAT | |
| | CTACCTGCCTGGACAGCATGGCCTGCAACGCGGGCATCCCGATGC | |
| | CGCCGGAAGCGAGAAGAATCATAATGGGGAAGGCCATCCAGCCTC | |
| | GCGTCGGGGAGCTTTTTGCAAAAGCCTAGGCCTCCAAAAAAGCCT | |
| | CCTCACTACTTCTGGAATAGCTCAGAGGCCGAGGCGGCCTCGGCCT | |
| | CTGCATAAATAAAAAAAATTAGTCAGCCATG | |
| 55 | AGCTTGGCCCATTGCATACGTTGTATCCATATCATAATATGTACAT | C-term PH |
| | TTATATTGGCTCATGTCCAACATTACCGCCATGTTGACATTGATTAT | domain |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGCCAGCAAT | |
| | TTTACGCAATTCGTGCTCGTGGACAACGGCGGCACGGGCGACGTG | |
| | ACCGTGGCCCCCAGCAACTTCGCCAATGGCATCGCCGAATGGATC | |
| | AGCAGCAACAGCAGGAGCCAGGCGTATAAAGTTACGTGCAGCGTC | |
| | AGACAGAGCAGCGCCCAGAACAGGAAATATACGATCAAGGTCGA | |
| | GGTTCCCAAGGGAGCTTGGAGGAGCTATCTTAATATGGAGCTGAC | |
| | CATCCCCATCTTCGCGACAAATTCAGACTGCGAGCTCATCGTGAAG | |
| | GCAATGCAGGGCCTCTTGAAAGATGGCAACCCCATCCCAAGCGCA | |
| | ATCGCGGCCAACTCAGGAATCTACTCCCAGAACTACCCCATCGTGC | |
| | AGATGGACAGCGGGAGGGATTTCCTCACGCTGCACGGTTTGCAGG | |
| | ACGACGAGGACCTCCAAGCTTTGCTCAAAGGCAGTCAACTTCTCA | |
| | AGGTCAAGAGCAGCTCCTGGAGGCGCGAACGGTTTTACAAGTTGC | |
| | AAGAAGATTGCAAAACAATTTGGCAAGAGAGCCGGAAAGTCATGA | |
| | GAACTCCCGAGAGCCAACTGTTCAGCATCGAGGACATCCAAGAAG | |
| | TCAGGATGGGCCATAGGACCGAGGGCTTGGAAAAATTCGCCAGGG | |
| | ACGTGCCCGAAGACCGATGCTTTAGCATCGTGTTCAAAGATCAGA | |
| | GGAACACGTTGGACTTGATCGCCCCCAGTCCGGCTGATGCCCAGC | |
| | ATTGGGTTCTGGGGCTCCATAAGATCATCCATCATAGCGGCAGCAT | |
| | GGACCAGAGGCAGAAACTCCAACATTGGATTCATAGTTGTCTTAG | |
| | GAAAGCCGACAAGAACAAGGACAACAAGATGAGCTTCAAGGAGT | |
| | TGCAGAATTTTCTTAAAGAGCTGAATATCCAGTAAGGATCCAAGCT | |
| | TATCGATACCGTCGACCTCGAGGGCCCAGATCTAATTCACCCCACC | |
| | AGTGCAGGCTGCCTATCAGAAAGTGGTGGCTGGTGTGGCTAATGC | |
| | CCTGGCCCACAAGTATCACTAAGCTCGCTTTCTTGCTGTCCAATTTC | |
| | TATTAAAGGTTCCTTTGTTCCCTAAGTCCAACTACTAAACTGGGGG | |
| | ATATTATGAAGGGCCTTGAGCATCTGGATTCTGCCTAATAAAAAAC | |
| | ATTTATTTTCATTGCAATGATGTATTTAAATTATTTCTGAATATTTT | |
| | ACTAAAAAGGGAATGTGGGAGGTCAGTGCATTTAAAACATAAAGA | |
| | AATGAAGAGCTAGTTCAAACCTTGGGAAAATACACTATATCTTAA | |
| | ACTCCATGAAAGAAGGTGAGGCTGCAAACAGCTAATGCACATTGG | |
| | CAACAGCCCCTGATGCCTATGCCTTATTCATCCCTCAGAAAAGGAT | |
| | TCAAGTAGAGGCTTGATTTGGAGGTTAAAGTTTTGCTATGCTGTAT | |
| | TTTACATTACTTATTGTTTTAGCTGTCCTCATGAATGTCTTTTCACT | |
| | ACCCATTTGCTTATCCTGCATCTCTCAGCCTTGACTCCACTCAGTTC | |
| | TCTTGCTTAGAGATACCACCTTTCCCCTGAAGTGTTCCTTCCATGTT | |
| | TTACGGCGAGATGGTTTCTCCTCGCCTGGCCACTCAGCCTTAGTTG | |
| | TCTCTGTTGTCTTATAGAGGTCTACTTGAAGAAGGAAAAACAGGG | |
| | GGCATGGTTTGACTGTCCTGTGAGCCCTTCTTCCCTGCCTCCCCCAC | |
| | TCACAGTGACCCGGAATCCCTCGACATGGCAGTCTAGATCATTCTT | |
| | GAAGACGAAAGGGCCTCGTGATACGCCTATTTTTATAGGTTAATGT | |
| | CATGATAATAATGGTTTCTTAGACGTCAGGTGGCACTTTTCGGGGA | |
| | AATGTGCGCGGAACCCCTATTTGTTTATTTTTCTAAATACATTCAA | |
| | ATATGTATCCGCTCATGAGACAATAACCCTGATAAATGCTTCAATA | |
| | ATATTGAAAAAGGAAGAGTATGAGTATTCAACATTTCCGTGTCGCC | |
| | CTTATTCCCTTTTTTGCGGCATTTTGCCTTCCTGTTTTTGCTCACCCA | |
| | GAAACGCTGGTGAAAGTAAAAGATGCTGAAGATCAGTTGGGTGCA | |
| | CGAGTGGGTTACATCGAACTGGATCTCAACAGCGGTAAGATCCTT | |
| | GAGAGTTTTCGCCCCGAAGAACGTTTTCCAATGATGAGCACTTTTA | |
| | AAGTTCTGCTATGTGGCGCGGTATTATCCCGTATTGACGCCGGGCA | |
| | AGAGCAACTCGGTCGCCGCATACACTATTCTCAGAATGACTTGGTT | |
| | GAGTACTCACCAGTCACAGAAAAGCATCTTACGGATGGCATGACA | |
| | GTAAGAGAATTATGCAGTGCTGCCATAACCATGAGTGATAACACT | |
| | GCGGCCAACTTACTTCTGACAACGATCGGAGGACCGAAGGAGCTA | |
| | ACCGCTTTTTTGCACAACATGGGGGATCATGTAACTCGCCTTGATC | |
| | GTTGGGAACCGGAGCTGAATGAAGCCATACCAAACGACGAGCGTG | |
| | ACACCACGATGCCTGTAGCAATGGCAACAACGTTGCGCAAACTAT | |
| | TAACTGGCGAACTACTTACTCTAGCTTCCCGGCAACAATTAATAGA | |
| | CTGGATGGAGGCGGATAAAGTTGCAGGACCACTTCTGCGCTCGGC | |
| | CCTTCCGGCTGGCTGGTTTATTGCTGATAAATCTGGAGCCGGTGAG | |
| | CGTGGGTCTCGCGGTATCATTGCAGCACTGGGGCCAGATGGTAAG | |
| | CCCTCCCGTATCGTAGTTATCTACACGACGGGGAGTCAGGCAACTA | |
| | TGGATGAACGAAATAGACAGATCGCTGAGATAGGTGCCTCACTGA | |
| | TTAAGCATTGGTAACTGTCAGACCAAGTTTACTCATATATACTTTA | |
| | GATTGATTTAAAACTTCATTTTTAATTTAAAAGGATCTAGGTGAAG | |
| | ATCCTTTTTGATAATCTCATGACCAAAATCCCTTAACGTGAGTTTTC | |
| | GTTCCACTGAGCGTCAGACCCCGTAGAAAAGATCAAAGGATCTTC | |
| | TTGAGATCCTTTTTTTCTGCGCGTAATCTGCTGCTTGCAAACAAAA | |
| | AAACCACCGCTACCAGCGGTGGTTTGTTTGCCGGATCAAGAGCTAC | |
| | CAACTCTTTTTCCGAAGGTAACTGGCTTCAGCAGAGCGCAGATACC | |
| | AAATACTGTTCTTCTAGTGTAGCCGTAGTTAGGCCACCACTTCAAG | |
| | AACTCTGTAGCACCGCCTACATACCTCGCTCTGCTAATCCTGTTAC | |
| | CAGTGGCTGCTGCCAGTGGCGATAAGTCGTGTCTTACCGGGTTGGA | |
| | CTCAAGACGATAGTTACCGGATAAGGCGCAGCGGTCGGGCTGAAC | |
| | GGGGGGTTCGTGCACACAGCCCAGCTTGGAGCGAACGACCTACAC | |
| | CGAACTGAGATACCTACAGCGTGAGCTATGAGAAAGCGCCACGCT | |
| | TCCCGAAGGGAGAAAGGCGGACAGGTATCCGGTAAGCGGCAGGG | |
| | TCGGAACAGGAGAGCGCACGAGGGAGCTTCCAGGGGGAAACGCC | |
| | TGGTATCTTTATAGTCCTGTCGGGTTTCGCCACCTCTGACTTGAGCG | |
| | TCGATTTTTGTGATGCTCGTCAGGGGGGCGGAGCCTATGGAAAAA | |
| | CGCCAGCAACGGATGCGCCGCGTGCGGCTGCTGGAGATGGCGGAC | |
| | GCGATGGATATGTTCTGCCAAGGGTTGGTTTGCGCATTCACAGTTC | |
| | TCCGCAAGAATTGATTGGCTCCAATTCTTGGAGTGGTGAATCCGTT | |
| | AGCGAGGTGCCGCCGGCTTCCATTCAGGTCGAGGTGGCCCGGCTC | |
| | CATGCACCGCGACGCAACGCGGGGAGGCAGACAAGGTATAGGGC | |
| | GGCGCCTACAATCCATGCCAACCCGTTCCATGTGCTCGCCGAGGCG | |
| | GCATAAATCCCCGTGACGATCAGCGGTCCAATGATCGAAGTTAGG | |
| | CTGGTAAGAGCCGCGAGCGATCCTTGAAGCTGTCCCTGATGGTCGT | |
| | CATCTACCTGCCTGGACAGCATGGCCTGCAACGCGGGCATCCCGAT | |
| | GCCGCCGGAAGCGAGAAGAATCATAATGGGGAAGGCCATCCAGCC | |
| | TCGCGTCGGGGAGCTTTTTGCAAAAGCCTAGGCCTCCAAAAAAGC | |
| | CTCCTCACTACTTCTGGAATAGCTCAGAGGCCGAGGCGGCCTCGGC | |
| | CTCTGCATAAATAAAAAAAATTAGTCAGCCATG | |
| 56 | AGCTTGGCCCATTGCATACGTTGTATCCATATCATAATATGTACAT | Myr (n-term; |
| | TTATATTGGCTCATGTCCAACATTACCGCCATGTTGACATTGATTAT | lyn pal⁻) |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCATC | |
| | AAATCAAAGAGGAAGGATAATCTGAATGACGATGAATCCCAGAAC | |
| | TACCCCATCGTGCAGATGGCCAGCAATTTTACGCAATTCGTGCTCG | |
| | TGGACAACGGCGGCACGGGCGACGTGACCGTGGCCCCCAGCAACT | |
| | TCGCCAATGGCATCGCCGAATGGATCAGCAGCAACAGCAGGAGCC | |
| | AGGCGTATAAAGTTACGTGCAGCGTCAGACAGAGCAGCGCCCAGA | |
| | ACAGGAAATATACGATCAAGGTCGAGGTTCCCAAGGGAGCTTGGA | |
| | GGAGCTATCTTAATATGGAGCTGACCATCCCCATCTTCGCGACAAA | |
| | TTCAGACTGCGAGCTCATCGTGAAGGCAATGCAGGGCCTCTTGAA | |
| | AGATGGCAACCCCATCCCAAGCGCAATCGCGGCCAACTCAGGAAT | |
| | CTACTAAGGATCCAAGCTTATCGATACCGTCGACCTCGAGGGCCCA | |
| | GATCTAATTCACCCCACCAGTGCAGGCTGCCTATCAGAAAGTGGTG | |
| | GCTGGTGTGGCTAATGCCCTGGCCCACAAGTATCACTAAGCTCGCT | |
| | TTCTTGCTGTCCAATTTCTATTAAAGGTTCCTTTGTTCCCTAAGTCC | |
| | AACTACTAAACTGGGGGATATTATGAAGGGCCTTGAGCATCTGGA | |
| | TTCTGCCTAATAAAAAACATTTATTTTCATTGCAATGATGTATTTAA | |
| | ATTATTTCTGAATATTTTACTAAAAAGGGAATGTGGGAGGTCAGTG | |
| | CATTTAAAACATAAAGAAATGAAGAGCTAGTTCAAACCTTGGGAA | |
| | AATACACTATATCTTAAACTCCATGAAAGAAGGTGAGGCTGCAAA | |
| | CAGCTAATGCACATTGGCAACAGCCCCTGATGCCTATGCCTTATTC | |
| | ATCCCTCAGAAAAGGATTCAAGTAGAGGCTTGATTTGGAGGTTAA | |
| | AGTTTTGCTATGCTGTATTTTACATTACTTATTGTTTTAGCTGTCCTC | |
| | ATGAATGTCTTTTCACTACCCATTTGCTTATCCTGCATCTCTCAGCC | |
| | TTGACTCCACTCAGTTCTCTTGCTTAGAGATACCACCTTTCCCCTGA | |
| | AGTGTTCCTTCCATGTTTTACGGCGAGATGGTTTCTCCTCGCCTGGC | |
| | CACTCAGCCTTAGTTGTCTCTGTTGTCTTATAGAGGTCTACTTGAAG | |
| | AAGGAAAAACAGGGGGCATGGTTTGACTGTCCTGTGAGCCCTTCTT | |
| | CCCTGCCTCCCCCACTCACAGTGACCCGGAATCCCTCGACATGGCA | |
| | GTCTAGATCATTCTTGAAGACGAAAGGGCCTCGTGATACGCCTATT | |
| | TTTATAGGTTAATGTCATGATAATAATGGTTTCTTAGACGTCAGGT | |
| | GGCACTTTTCGGGGAAATGTGCGCGGAACCCCTATTTGTTTATTTT | |
| | TCTAAATACATTCAAATATGTATCCGCTCATGAGACAATAACCCTG | |
| | ATAAATGCTTCAATAATATTGAAAAAGGAAGAGTATGAGTATTCA | |
| | ACATTTCCGTGTCGCCCTTATTCCCTTTTTTGCGGCATTTTGCCTTC | |
| | CTGTTTTTGCTCACCCAGAAACGCTGGTGAAAGTAAAAGATGCTGA | |
| | AGATCAGTTGGGTGCACGAGTGGGTTACATCGAACTGGATCTCAA | |
| | CAGCGGTAAGATCCTTGAGAGTTTTCGCCCCGAAGAACGTTTTCCA | |
| | ATGATGAGCACTTTTAAAGTTCTGCTATGTGGCGCGGTATTATCCC | |
| | GTATTGACGCCGGGCAAGAGCAACTCGGTCGCCGCATACACTATT | |
| | CTCAGAATGACTTGGTTGAGTACTCACCAGTCACAGAAAAGCATCT | |
| | TACGGATGGCATGACAGTAAGAGAATTATGCAGTGCTGCCATAAC | |
| | CATGAGTGATAACACTGCGGCCAACTTACTTCTGACAACGATCGG | |
| | AGGACCGAAGGAGCTAACCGCTTTTTTGCACAACATGGGGGATCA | |
| | TGTAACTCGCCTTGATCGTTGGGAACCGGAGCTGAATGAAGCCAT | |
| | ACCAAACGACGAGCGTGACACCACGATGCCTGTAGCAATGGCAAC | |
| | AACGTTGCGCAAACTATTAACTGGCGAACTACTTACTCTAGCTTCC | |
| | CGGCAACAATTAATAGACTGGATGGAGGCGGATAAAGTTGCAGGA | |
| | CCACTTCTGCGCTCGGCCCTTCCGGCTGGCTGGTTTATTGCTGATA | |
| | AATCTGGAGCCGGTGAGCGTGGGTCTCGCGGTATCATTGCAGCACT | |
| | GGGGCCAGATGGTAAGCCCTCCCGTATCGTAGTTATCTACACGACG | |
| | GGGAGTCAGGCAACTATGGATGAACGAAATAGACAGATCGCTGAG | |
| | ATAGGTGCCTCACTGATTAAGCATTGGTAACTGTCAGACCAAGTTT | |
| | ACTCATATATACTTTAGATTGATTTAAAACTTCATTTTTAATTTAAA | |
| | AGGATCTAGGTGAAGATCCTTTTTGATAATCTCATGACCAAAATCC | |
| | CTTAACGTGAGTTTTCGTTCCACTGAGCGTCAGACCCCGTAGAAAA | |
| | GATCAAAGGATCTTCTTGAGATCCTTTTTTTCTGCGCGTAATCTGCT | |
| | GCTTGCAAACAAAAAAACCACCGCTACCAGCGGTGGTTTGTTTGCC | |
| | GGATCAAGAGCTACCAACTCTTTTTCCGAAGGTAACTGGCTTCAGC | |
| | AGAGCGCAGATACCAAATACTGTTCTTCTAGTGTAGCCGTAGTTAG | |
| | GCCACCACTTCAAGAACTCTGTAGCACCGCCTACATACCTCGCTCT | |
| | GCTAATCCTGTTACCAGTGGCTGCTGCCAGTGGCGATAAGTCGTGT | |
| | CTTACCGGGTTGGACTCAAGACGATAGTTACCGGATAAGGCGCAG | |
| | CGGTCGGGCTGAACGGGGGGTTCGTGCACACAGCCCAGCTTGGAG | |
| | CGAACGACCTACACCGAACTGAGATACCTACAGCGTGAGCTATGA | |
| | GAAAGCGCCACGCTTCCCGAAGGGAGAAAGGCGGACAGGTATCCG | |
| | GTAAGCGGCAGGGTCGGAACAGGAGAGCGCACGAGGGAGCTTCC | |
| | AGGGGGAAACGCCTGGTATCTTTATAGTCCTGTCGGGTTTCGCCAC | |
| | CTCTGACTTGAGCGTCGATTTTTGTGATGCTCGTCAGGGGGGCGGA | |
| | GCCTATGGAAAAACGCCAGCAACGGATGCGCCGCGTGCGGCTGCT | |
| | GGAGATGGCGGACGCGATGGATATGTTCTGCCAAGGGTTGGTTTG | |
| | CGCATTCACAGTTCTCCGCAAGAATTGATTGGCTCCAATTCTTGGA | |
| | GTGGTGAATCCGTTAGCGAGGTGCCGCCGGCTTCCATTCAGGTCGA | |
| | GGTGGCCCGGCTCCATGCACCGCGACGCAACGCGGGGAGGCAGAC | |
| | AAGGTATAGGGCGGCGCCTACAATCCATGCCAACCCGTTCCATGT | |
| | GCTCGCCGAGGCGGCATAAATCCCCGTGACGATCAGCGGTCCAAT | |
| | GATCGAAGTTAGGCTGGTAAGAGCCGCGAGCGATCCTTGAAGCTG | |
| | TCCCTGATGGTCGTCATCTACCTGCCTGGACAGCATGGCCTGCAAC | |
| | GCGGGCATCCCGATGCCGCCGGAAGCGAGAAGAATCATAATGGGG | |
| | AAGGCCATCCAGCCTCGCGTCGGGGAGCTTTTTGCAAAAGCCTAG | |
| | GCCTCCAAAAAAGCCTCCTCACTACTTCTGGAATAGCTCAGAGGCC | |
| | GAGGCGGCCTCGGCCTCTGCATAAATAAAAAAAATTAGTCAGCCA | |
| | TG | |
| 57 | AGCTTGGCCCATTGCATACGTTGTATCCATATCATAATATGTACAT | Single Pal, |
| | TTATATTGGCTCATGTCCAACATTACCGCCATGTTGACATTGATTAT | from GNA12 |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGTCCGGGGTG | |
| | GTGCGGACCCTCAGCCGCTGCCTGCTGCCGGCCGAGGCCGGCTCCC | |
| | AGAACTACCCCATCGTGCAGATGGCCAGCAATTTTACGCAATTCGT | |
| | GCTCGTGGACAACGGCGGCACGGGCGACGTGACCGTGGCCCCCAG | |
| | CAACTTCGCCAATGGCATCGCCGAATGGATCAGCAGCAACAGCAG | |
| | GAGCCAGGCGTATAAAGTTACGTGCAGCGTCAGACAGAGCAGCGC | |
| | CCAGAACAGGAAATATACGATCAAGGTCGAGGTTCCCAAGGGAGC | |
| | TTGGAGGAGCTATCTTAATATGGAGCTGACCATCCCCATCTTCGCG | |
| | ACAAATTCAGACTGCGAGCTCATCGTGAAGGCAATGCAGGGCCTC | |
| | TTGAAAGATGGCAACCCCATCCCAAGCGCAATCGCGGCCAACTCA | |
| | GGAATCTACTAAGGATCCAAGCTTATCGATACCGTCGACCTCGAG | |
| | GGCCCAGATCTAATTCACCCCACCAGTGCAGGCTGCCTATCAGAA | |
| | AGTGGTGGCTGGTGTGGCTAATGCCCTGGCCCACAAGTATCACTAA | |
| | GCTCGCTTTCTTGCTGTCCAATTTCTATTAAAGGTTCCTTTGTTCCC | |
| | TAAGTCCAACTACTAAACTGGGGGATATTATGAAGGGCCTTGAGC | |
| | ATCTGGATTCTGCCTAATAAAAAACATTTATTTTCATTGCAATGAT | |
| | GTATTTAAATTATTTCTGAATATTTTACTAAAAAGGGAATGTGGGA | |
| | GGTCAGTGCATTTAAAACATAAAGAAATGAAGAGCTAGTTCAAAC | |
| | CTTGGGAAAATACACTATATCTTAAACTCCATGAAAGAAGGTGAG | |
| | GCTGCAAACAGCTAATGCACATTGGCAACAGCCCCTGATGCCTAT | |
| | GCCTTATTCATCCCTCAGAAAAGGATTCAAGTAGAGGCTTGATTTG | |
| | GAGGTTAAAGTTTTGCTATGCTGTATTTTACATTACTTATTGTTTTA | |
| | GCTGTCCTCATGAATGTCTTTTCACTACCCATTTGCTTATCCTGCAT | |
| | CTCTCAGCCTTGACTCCACTCAGTTCTCTTGCTTAGAGATACCACCT | |
| | TTCCCCTGAAGTGTTCCTTCCATGTTTTACGGCGAGATGGTTTCTCC | |
| | TCGCCTGGCCACTCAGCCTTAGTTGTCTCTGTTGTCTTATAGAGGTC | |
| | TACTTGAAGAAGGAAAAACAGGGGGCATGGTTTGACTGTCCTGTG | |
| | AGCCCTTCTTCCCTGCCTCCCCCACTCACAGTGACCCGGAATCCCT | |
| | CGACATGGCAGTCTAGATCATTCTTGAAGACGAAAGGGCCTCGTG | |
| | ATACGCCTATTTTTATAGGTTAATGTCATGATAATAATGGTTTCTTA | |
| | GACGTCAGGTGGCACTTTTCGGGGAAATGTGCGCGGAACCCCTATT | |
| | TGTTTATTTTTCTAAATACATTCAAATATGTATCCGCTCATGAGACA | |
| | ATAACCCTGATAAATGCTTCAATAATATTGAAAAAGGAAGAGTAT | |
| | GAGTATTCAACATTTCCGTGTCGCCCTTATTCCCTTTTTTGCGGCAT | |
| | TTTGCCTTCCTGTTTTTGCTCACCCAGAAACGCTGGTGAAAGTAAA | |
| | AGATGCTGAAGATCAGTTGGGTGCACGAGTGGGTTACATCGAACT | |
| | GGATCTCAACAGCGGTAAGATCCTTGAGAGTTTTCGCCCCGAAGA | |
| | ACGTTTTCCAATGATGAGCACTTTTAAAGTTCTGCTATGTGGCGCG | |
| | GTATTATCCCGTATTGACGCCGGGCAAGAGCAACTCGGTCGCCGC | |
| | ATACACTATTCTCAGAATGACTTGGTTGAGTACTCACCAGTCACAG | |
| | AAAAGCATCTTACGGATGGCATGACAGTAAGAGAATTATGCAGTG | |
| | CTGCCATAACCATGAGTGATAACACTGCGGCCAACTTACTTCTGAC | |
| | AACGATCGGAGGACCGAAGGAGCTAACCGCTTTTTTGCACAACAT | |
| | GGGGGATCATGTAACTCGCCTTGATCGTTGGGAACCGGAGCTGAA | |
| | TGAAGCCATACCAAACGACGAGCGTGACACCACGATGCCTGTAGC | |
| | AATGGCAACAACGTTGCGCAAACTATTAACTGGCGAACTACTTACT | |
| | CTAGCTTCCCGGCAACAATTAATAGACTGGATGGAGGCGGATAAA | |
| | GTTGCAGGACCACTTCTGCGCTCGGCCCTTCCGGCTGGCTGGTTTA | |
| | TTGCTGATAAATCTGGAGCCGGTGAGCGTGGGTCTCGCGGTATCAT | |
| | TGCAGCACTGGGGCCAGATGGTAAGCCCTCCCGTATCGTAGTTATC | |
| | TACACGACGGGGAGTCAGGCAACTATGGATGAACGAAATAGACAG | |
| | ATCGCTGAGATAGGTGCCTCACTGATTAAGCATTGGTAACTGTCAG | |
| | ACCAAGTTTACTCATATATACTTTAGATTGATTTAAAACTTCATTTT | |
| | TAATTTAAAAGGATCTAGGTGAAGATCCTTTTTGATAATCTCATGA | |
| | CCAAAATCCCTTAACGTGAGTTTTCGTTCCACTGAGCGTCAGACCC | |
| | CGTAGAAAAGATCAAAGGATCTTCTTGAGATCCTTTTTTTCTGCGC | |
| | GTAATCTGCTGCTTGCAAACAAAAAAACCACCGCTACCAGCGGTG | |
| | GTTTGTTTGCCGGATCAAGAGCTACCAACTCTTTTTCCGAAGGTAA | |
| | CTGGCTTCAGCAGAGCGCAGATACCAAATACTGTTCTTCTAGTGTA | |
| | GCCGTAGTTAGGCCACCACTTCAAGAACTCTGTAGCACCGCCTACA | |
| | TACCTCGCTCTGCTAATCCTGTTACCAGTGGCTGCTGCCAGTGGCG | |
| | ATAAGTCGTGTCTTACCGGGTTGGACTCAAGACGATAGTTACCGGA | |
| | TAAGGCGCAGCGGTCGGGCTGAACGGGGGGTTCGTGCACACAGCC | |
| | CAGCTTGGAGCGAACGACCTACACCGAACTGAGATACCTACAGCG | |
| | TGAGCTATGAGAAAGCGCCACGCTTCCCGAAGGGAGAAAGGCGGA | |
| | CAGGTATCCGGTAAGCGGCAGGGTCGGAACAGGAGAGCGCACGA | |
| | GGGAGCTTCCAGGGGGAAACGCCTGGTATCTTTATAGTCCTGTCGG | |
| | GTTTCGCCACCTCTGACTTGAGCGTCGATTTTTGTGATGCTCGTCAG | |
| | GGGGGCGGAGCCTATGGAAAAACGCCAGCAACGGATGCGCCGCGT | |
| | GCGGCTGCTGGAGATGGCGGACGCGATGGATATGTTCTGCCAAGG | |
| | GTTGGTTTGCGCATTCACAGTTCTCCGCAAGAATTGATTGGCTCCA | |
| | ATTCTTGGAGTGGTGAATCCGTTAGCGAGGTGCCGCCGGCTTCCAT | |
| | TCAGGTCGAGGTGGCCCGGCTCCATGCACCGCGACGCAACGCGGG | |
| | GAGGCAGACAAGGTATAGGGCGGCGCCTACAATCCATGCCAACCC | |
| | GTTCCATGTGCTCGCCGAGGCGGCATAAATCCCCGTGACGATCAGC | |
| | GGTCCAATGATCGAAGTTAGGCTGGTAAGAGCCGCGAGCGATCCT | |
| | TGAAGCTGTCCCTGATGGTCGTCATCTACCTGCCTGGACAGCATGG | |
| | CCTGCAACGCGGGCATCCCGATGCCGCCGGAAGCGAGAAGAATCA | |
| | TAATGGGGAAGGCCATCCAGCCTCGCGTCGGGGAGCTTTTTGCAA | |
| | AAGCCTAGGCCTCCAAAAAAGCCTCCTCACTACTTCTGGAATAGCT | |
| | CAGAGGCCGAGGCGGCCTCGGCCTCTGCATAAATAAAAAAAATTA | |
| | GTCAGCCATG | |
| 58 | AGCTTGGCCCATTGCATACGTTGTATCCATATCATAATATGTACAT | Double Pal, |
| | TTATATTGGCTCATGTCCAACATTACCGCCATGTTGACATTGATTAT | from GNA13 |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGCGGACTTC | |
| | CTGCCGTCGCGGTCCGTGCTGTCCGTGTGCTTCCCCGGCTGCCTGC | |
| | TGACGAGTTCCCAGAACTACCCCATCGTGCAGATGGCCAGCAATTT | |
| | TACGCAATTCGTGCTCGTGGACAACGGCGGCACGGGCGACGTGAC | |
| | CGTGGCCCCCAGCAACTTCGCCAATGGCATCGCCGAATGGATCAG | |
| | CAGCAACAGCAGGAGCCAGGCGTATAAAGTTACGTGCAGCGTCAG | |
| | ACAGAGCAGCGCCCAGAACAGGAAATATACGATCAAGGTCGAGGT | |
| | TCCCAAGGGAGCTTGGAGGAGCTATCTTAATATGGAGCTGACCAT | |
| | CCCCATCTTCGCGACAAATTCAGACTGCGAGCTCATCGTGAAGGCA | |
| | ATGCAGGGCCTCTTGAAAGATGGCAACCCCATCCCAAGCGCAATC | |
| | GCGGCCAACTCAGGAATCTACTAAGGATCCAAGCTTATCGATACC | |
| | GTCGACCTCGAGGGCCCAGATCTAATTCACCCCACCAGTGCAGGCT | |
| | GCCTATCAGAAAGTGGTGGCTGGTGTGGCTAATGCCCTGGCCCAC | |
| | AAGTATCACTAAGCTCGCTTTCTTGCTGTCCAATTTCTATTAAAGGT | |
| | TCCTTTGTTCCCTAAGTCCAACTACTAAACTGGGGGATATTATGAA | |
| | GGGCCTTGAGCATCTGGATTCTGCCTAATAAAAAACATTTATTTTC | |
| | ATTGCAATGATGTATTTAAATTATTTCTGAATATTTTACTAAAAAG | |
| | GGAATGTGGGAGGTCAGTGCATTTAAAACATAAAGAAATGAAGAG | |
| | CTAGTTCAAACCTTGGGAAAATACACTATATCTTAAACTCCATGAA | |
| | AGAAGGTGAGGCTGCAAACAGCTAATGCACATTGGCAACAGCCCC | |
| | TGATGCCTATGCCTTATTCATCCCTCAGAAAAGGATTCAAGTAGAG | |
| | GCTTGATTTGGAGGTTAAAGTTTTGCTATGCTGTATTTTACATTACT | |
| | TATTGTTTTAGCTGTCCTCATGAATGTCTTTTCACTACCCATTTGCT | |
| | TATCCTGCATCTCTCAGCCTTGACTCCACTCAGTTCTCTTGCTTAGA | |
| | GATACCACCTTTCCCCTGAAGTGTTCCTTCCATGTTTTACGGCGAG | |
| | ATGGTTTCTCCTCGCCTGGCCACTCAGCCTTAGTTGTCTCTGTTGTC | |
| | TTATAGAGGTCTACTTGAAGAAGGAAAAACAGGGGGCATGGTTTG | |
| | ACTGTCCTGTGAGCCCTTCTTCCCTGCCTCCCCCACTCACAGTGACC | |
| | CGGAATCCCTCGACATGGCAGTCTAGATCATTCTTGAAGACGAAA | |
| | GGGCCTCGTGATACGCCTATTTTTATAGGTTAATGTCATGATAATA | |
| | ATGGTTTCTTAGACGTCAGGTGGCACTTTTCGGGGAAATGTGCGCG | |
| | GAACCCCTATTTGTTTATTTTTCTAAATACATTCAAATATGTATCCG | |
| | CTCATGAGACAATAACCCTGATAAATGCTTCAATAATATTGAAAA | |
| | AGGAAGAGTATGAGTATTCAACATTTCCGTGTCGCCCTTATTCCCT | |
| | TTTTTGCGGCATTTTGCCTTCCTGTTTTTGCTCACCCAGAAACGCTG | |
| | GTGAAAGTAAAAGATGCTGAAGATCAGTTGGGTGCACGAGTGGGT | |
| | TACATCGAACTGGATCTCAACAGCGGTAAGATCCTTGAGAGTTTTC | |
| | GCCCCGAAGAACGTTTTCCAATGATGAGCACTTTTAAAGTTCTGCT | |
| | ATGTGGCGCGGTATTATCCCGTATTGACGCCGGGCAAGAGCAACT | |
| | CGGTCGCCGCATACACTATTCTCAGAATGACTTGGTTGAGTACTCA | |
| | CCAGTCACAGAAAAGCATCTTACGGATGGCATGACAGTAAGAGAA | |
| | TTATGCAGTGCTGCCATAACCATGAGTGATAACACTGCGGCCAACT | |
| | TACTTCTGACAACGATCGGAGGACCGAAGGAGCTAACCGCTTTTTT | |
| | GCACAACATGGGGGATCATGTAACTCGCCTTGATCGTTGGGAACC | |
| | GGAGCTGAATGAAGCCATACCAAACGACGAGCGTGACACCACGAT | |
| | GCCTGTAGCAATGGCAACAACGTTGCGCAAACTATTAACTGGCGA | |
| | ACTACTTACTCTAGCTTCCCGGCAACAATTAATAGACTGGATGGAG | |
| | GCGGATAAAGTTGCAGGACCACTTCTGCGCTCGGCCCTTCCGGCTG | |
| | GCTGGTTTATTGCTGATAAATCTGGAGCCGGTGAGCGTGGGTCTCG | |
| | CGGTATCATTGCAGCACTGGGGCCAGATGGTAAGCCCTCCCGTATC | |
| | GTAGTTATCTACACGACGGGGAGTCAGGCAACTATGGATGAACGA | |
| | AATAGACAGATCGCTGAGATAGGTGCCTCACTGATTAAGCATTGG | |
| | TAACTGTCAGACCAAGTTTACTCATATATACTTTAGATTGATTTAA | |
| | AACTTCATTTTTAATTTAAAAGGATCTAGGTGAAGATCCTTTTTGAT | |
| | AATCTCATGACCAAAATCCCTTAACGTGAGTTTTCGTTCCACTGAG | |
| | CGTCAGACCCCGTAGAAAAGATCAAAGGATCTTCTTGAGATCCTTT | |
| | TTTTCTGCGCGTAATCTGCTGCTTGCAAACAAAAAAACCACCGCTA | |
| | CCAGCGGTGGTTTGTTTGCCGGATCAAGAGCTACCAACTCTTTTTC | |
| | CGAAGGTAACTGGCTTCAGCAGAGCGCAGATACCAAATACTGTTC | |
| | TTCTAGTGTAGCCGTAGTTAGGCCACCACTTCAAGAACTCTGTAGC | |
| | ACCGCCTACATACCTCGCTCTGCTAATCCTGTTACCAGTGGCTGCT | |
| | GCCAGTGGCGATAAGTCGTGTCTTACCGGGTTGGACTCAAGACGA | |
| | TAGTTACCGGATAAGGCGCAGCGGTCGGGCTGAACGGGGGGTTCG | |
| | TGCACACAGCCCAGCTTGGAGCGAACGACCTACACCGAACTGAGA | |
| | TACCTACAGCGTGAGCTATGAGAAAGCGCCACGCTTCCCGAAGGG | |
| | AGAAAGGCGGACAGGTATCCGGTAAGCGGCAGGGTCGGAACAGG | |
| | AGAGCGCACGAGGGAGCTTCCAGGGGGAAACGCCTGGTATCTTTA | |
| | TAGTCCTGTCGGGTTTCGCCACCTCTGACTTGAGCGTCGATTTTTGT | |
| | GATGCTCGTCAGGGGGGCGGAGCCTATGGAAAAACGCCAGCAACG | |
| | GATGCGCCGCGTGCGGCTGCTGGAGATGGCGGACGCGATGGATAT | |
| | GTTCTGCCAAGGGTTGGTTTGCGCATTCACAGTTCTCCGCAAGAAT | |
| | TGATTGGCTCCAATTCTTGGAGTGGTGAATCCGTTAGCGAGGTGCC | |
| | GCCGGCTTCCATTCAGGTCGAGGTGGCCCGGCTCCATGCACCGCGA | |
| | CGCAACGCGGGGAGGCAGACAAGGTATAGGGCGGCGCCTACAATC | |
| | CATGCCAACCCGTTCCATGTGCTCGCCGAGGCGGCATAAATCCCCG | |
| | TGACGATCAGCGGTCCAATGATCGAAGTTAGGCTGGTAAGAGCCG | |
| | CGAGCGATCCTTGAAGCTGTCCCTGATGGTCGTCATCTACCTGCCT | |
| | GGACAGCATGGCCTGCAACGCGGGCATCCCGATGCCGCCGGAAGC | |
| | GAGAAGAATCATAATGGGGAAGGCCATCCAGCCTCGCGTCGGGGA | |
| | GCTTTTTGCAAAAGCCTAGGCCTCCAAAAAAGCCTCCTCACTACTT | |
| | CTGGAATAGCTCAGAGGCCGAGGCGGCCTCGGCCTCTGCATAAAT | |
| | AAAAAAAATTAGTCAGCCATG | |
| 59 | AGCTTGGCCCATTGCATACGTTGTATCCATATCATAATATGTACAT | Triple Pal |
| | TTATATTGGCTCATGTCCAACATTACCGCCATGTTGACATTGATTAT | from GNA15 |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGCCCGCTCG | |
| | CTGACCTGGCGCTGCTGCCCCTGGTGCCTGACGGAGGATGAGAAG | |
| | GCCGCCGCCTCCCAGAACTACCCCATCGTGCAGATGGCCAGCAATT | |
| | TTACGCAATTCGTGCTCGTGGACAACGGCGGCACGGGCGACGTGA | |
| | CCGTGGCCCCCAGCAACTTCGCCAATGGCATCGCCGAATGGATCA | |
| | GCAGCAACAGCAGGAGCCAGGCGTATAAAGTTACGTGCAGCGTCA | |
| | GACAGAGCAGCGCCCAGAACAGGAAATATACGATCAAGGTCGAG | |
| | GTTCCCAAGGGAGCTTGGAGGAGCTATCTTAATATGGAGCTGACC | |
| | ATCCCCATCTTCGCGACAAATTCAGACTGCGAGCTCATCGTGAAGG | |
| | CAATGCAGGGCCTCTTGAAAGATGGCAACCCCATCCCAAGCGCAA | |
| | TCGCGGCCAACTCAGGAATCTACTAAGGATCCAAGCTTATCGATAC | |
| | CGTCGACCTCGAGGGCCCAGATCTAATTCACCCCACCAGTGCAGG | |
| | CTGCCTATCAGAAAGTGGTGGCTGGTGTGGCTAATGCCCTGGCCCA | |
| | CAAGTATCACTAAGCTCGCTTTCTTGCTGTCCAATTTCTATTAAAG | |
| | GTTCCTTTGTTCCCTAAGTCCAACTACTAAACTGGGGGATATTATG | |
| | AAGGGCCTTGAGCATCTGGATTCTGCCTAATAAAAAACATTTATTT | |
| | TCATTGCAATGATGTATTTAAATTATTTCTGAATATTTTACTAAAAA | |
| | GGGAATGTGGGAGGTCAGTGCATTTAAAACATAAAGAAATGAAGA | |
| | GCTAGTTCAAACCTTGGGAAAATACACTATATCTTAAACTCCATGA | |
| | AAGAAGGTGAGGCTGCAAACAGCTAATGCACATTGGCAACAGCCC | |
| | CTGATGCCTATGCCTTATTCATCCCTCAGAAAAGGATTCAAGTAGA | |
| | GGCTTGATTTGGAGGTTAAAGTTTTGCTATGCTGTATTTTACATTAC | |
| | TTATTGTTTTAGCTGTCCTCATGAATGTCTTTTCACTACCCATTTGC | |
| | TTATCCTGCATCTCTCAGCCTTGACTCCACTCAGTTCTCTTGCTTAG | |
| | AGATACCACCTTTCCCCTGAAGTGTTCCTTCCATGTTTTACGGCGA | |
| | GATGGTTTCTCCTCGCCTGGCCACTCAGCCTTAGTTGTCTCTGTTGT | |
| | CTTATAGAGGTCTACTTGAAGAAGGAAAAACAGGGGGCATGGTTT | |
| | GACTGTCCTGTGAGCCCTTCTTCCCTGCCTCCCCCACTCACAGTGA | |
| | CCCGGAATCCCTCGACATGGCAGTCTAGATCATTCTTGAAGACGAA | |
| | AGGGCCTCGTGATACGCCTATTTTTATAGGTTAATGTCATGATAAT | |
| | AATGGTTTCTTAGACGTCAGGTGGCACTTTTCGGGGAAATGTGCGC | |
| | GGAACCCCTATTTGTTTATTTTTCTAAATACATTCAAATATGTATCC | |
| | GCTCATGAGACAATAACCCTGATAAATGCTTCAATAATATTGAAA | |
| | AAGGAAGAGTATGAGTATTCAACATTTCCGTGTCGCCCTTATTCCC | |
| | TTTTTTGCGGCATTTTGCCTTCCTGTTTTTGCTCACCCAGAAACGCT | |
| | GGTGAAAGTAAAAGATGCTGAAGATCAGTTGGGTGCACGAGTGGG | |
| | TTACATCGAACTGGATCTCAACAGCGGTAAGATCCTTGAGAGTTTT | |
| | CGCCCCGAAGAACGTTTTCCAATGATGAGCACTTTTAAAGTTCTGC | |
| | TATGTGGCGCGGTATTATCCCGTATTGACGCCGGGCAAGAGCAACT | |
| | CGGTCGCCGCATACACTATTCTCAGAATGACTTGGTTGAGTACTCA | |
| | CCAGTCACAGAAAAGCATCTTACGGATGGCATGACAGTAAGAGAA | |
| | TTATGCAGTGCTGCCATAACCATGAGTGATAACACTGCGGCCAACT | |
| | TACTTCTGACAACGATCGGAGGACCGAAGGAGCTAACCGCTTTTTT | |
| | GCACAACATGGGGGATCATGTAACTCGCCTTGATCGTTGGGAACC | |
| | GGAGCTGAATGAAGCCATACCAAACGACGAGCGTGACACCACGAT | |
| | GCCTGTAGCAATGGCAACAACGTTGCGCAAACTATTAACTGGCGA | |
| | ACTACTTACTCTAGCTTCCCGGCAACAATTAATAGACTGGATGGAG | |
| | GCGGATAAAGTTGCAGGACCACTTCTGCGCTCGGCCCTTCCGGCTG | |
| | GCTGGTTTATTGCTGATAAATCTGGAGCCGGTGAGCGTGGGTCTCG | |
| | CGGTATCATTGCAGCACTGGGGCCAGATGGTAAGCCCTCCCGTATC | |
| | GTAGTTATCTACACGACGGGGAGTCAGGCAACTATGGATGAACGA | |
| | AATAGACAGATCGCTGAGATAGGTGCCTCACTGATTAAGCATTGG | |
| | TAACTGTCAGACCAAGTTTACTCATATATACTTTAGATTGATTTAA | |
| | AACTTCATTTTTAATTTAAAAGGATCTAGGTGAAGATCCTTTTTGAT | |
| | AATCTCATGACCAAAATCCCTTAACGTGAGTTTTCGTTCCACTGAG | |
| | CGTCAGACCCCGTAGAAAAGATCAAAGGATCTTCTTGAGATCCTTT | |
| | TTTTCTGCGCGTAATCTGCTGCTTGCAAACAAAAAAACCACCGCTA | |
| | CCAGCGGTGGTTTGTTTGCCGGATCAAGAGCTACCAACTCTTTTTC | |
| | CGAAGGTAACTGGCTTCAGCAGAGCGCAGATACCAAATACTGTTC | |
| | TTCTAGTGTAGCCGTAGTTAGGCCACCACTTCAAGAACTCTGTAGC | |
| | ACCGCCTACATACCTCGCTCTGCTAATCCTGTTACCAGTGGCTGCT | |
| | GCCAGTGGCGATAAGTCGTGTCTTACCGGGTTGGACTCAAGACGA | |
| | TAGTTACCGGATAAGGCGCAGCGGTCGGGCTGAACGGGGGGTTCG | |
| | TGCACACAGCCCAGCTTGGAGCGAACGACCTACACCGAACTGAGA | |
| | TACCTACAGCGTGAGCTATGAGAAAGCGCCACGCTTCCCGAAGGG | |
| | AGAAAGGCGGACAGGTATCCGGTAAGCGGCAGGGTCGGAACAGG | |
| | AGAGCGCACGAGGGAGCTTCCAGGGGGAAACGCCTGGTATCTTTA | |
| | TAGTCCTGTCGGGTTTCGCCACCTCTGACTTGAGCGTCGATTTTTGT | |
| | GATGCTCGTCAGGGGGGCGGAGCCTATGGAAAAACGCCAGCAACG | |
| | GATGCGCCGCGTGCGGCTGCTGGAGATGGCGGACGCGATGGATAT | |
| | GTTCTGCCAAGGGTTGGTTTGCGCATTCACAGTTCTCCGCAAGAAT | |
| | TGATTGGCTCCAATTCTTGGAGTGGTGAATCCGTTAGCGAGGTGCC | |
| | GCCGGCTTCCATTCAGGTCGAGGTGGCCCGGCTCCATGCACCGCGA | |
| | CGCAACGCGGGGAGGCAGACAAGGTATAGGGCGGCGCCTACAATC | |
| | CATGCCAACCCGTTCCATGTGCTCGCCGAGGCGGCATAAATCCCCG | |
| | TGACGATCAGCGGTCCAATGATCGAAGTTAGGCTGGTAAGAGCCG | |
| | CGAGCGATCCTTGAAGCTGTCCCTGATGGTCGTCATCTACCTGCCT | |
| | GGACAGCATGGCCTGCAACGCGGGCATCCCGATGCCGCCGGAAGC | |
| | GAGAAGAATCATAATGGGGAAGGCCATCCAGCCTCGCGTCGGGGA | |
| | GCTTTTTGCAAAAGCCTAGGCCTCCAAAAAAGCCTCCTCACTACTT | |
| | CTGGAATAGCTCAGAGGCCGAGGCGGCCTCGGCCTCTGCATAAAT | |
| | AAAAAAAATTAGTCAGCCATG | |
| 60 | AGCTTGGCCCATTGCATACGTTGTATCCATATCATAATATGTACAT | Myr-pal (lyn), |
| | TTATATTGGCTCATGTCCAACATTACCGCCATGTTGACATTGATTAT | N-term |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCTGCATC | |
| | AAATCAAAGAGGAAGGATAATCTGAATGACGATGAATCCCAGAAC | |
| | TACCCCATCGTGCAGATGGCCAGCAATTTTACGCAATTCGTGCTCG | |
| | TGGACAACGGCGGCACGGGCGACGTGACCGTGGCCCCCAGCAACT | |
| | TCGCCAATGGCATCGCCGAATGGATCAGCAGCAACAGCAGGAGCC | |
| | AGGCGTATAAAGTTACGTGCAGCGTCAGACAGAGCAGCGCCCAGA | |
| | ACAGGAAATATACGATCAAGGTCGAGGTTCCCAAGGGAGCTTGGA | |
| | GGAGCTATCTTAATATGGAGCTGACCATCCCCATCTTCGCGACAAA | |
| | TTCAGACTGCGAGCTCATCGTGAAGGCAATGCAGGGCCTCTTGAA | |
| | AGATGGCAACCCCATCCCAAGCGCAATCGCGGCCAACTCAGGAAT | |
| | CTACTAAGGATCCAAGCTTATCGATACCGTCGACCTCGAGGGCCCA | |
| | GATCTAATTCACCCCACCAGTGCAGGCTGCCTATCAGAAAGTGGTG | |
| | GCTGGTGTGGCTAATGCCCTGGCCCACAAGTATCACTAAGCTCGCT | |
| | TTCTTGCTGTCCAATTTCTATTAAAGGTTCCTTTGTTCCCTAAGTCC | |
| | AACTACTAAACTGGGGGATATTATGAAGGGCCTTGAGCATCTGGA | |
| | TTCTGCCTAATAAAAAACATTTATTTTCATTGCAATGATGTATTTAA | |
| | ATTATTTCTGAATATTTTACTAAAAAGGGAATGTGGGAGGTCAGTG | |
| | CATTTAAAACATAAAGAAATGAAGAGCTAGTTCAAACCTTGGGAA | |
| | AATACACTATATCTTAAACTCCATGAAAGAAGGTGAGGCTGCAAA | |
| | CAGCTAATGCACATTGGCAACAGCCCCTGATGCCTATGCCTTATTC | |
| | ATCCCTCAGAAAAGGATTCAAGTAGAGGCTTGATTTGGAGGTTAA | |
| | AGTTTTGCTATGCTGTATTTTACATTACTTATTGTTTTAGCTGTCCTC | |
| | ATGAATGTCTTTTCACTACCCATTTGCTTATCCTGCATCTCTCAGCC | |
| | TTGACTCCACTCAGTTCTCTTGCTTAGAGATACCACCTTTCCCCTGA | |
| | AGTGTTCCTTCCATGTTTTACGGCGAGATGGTTTCTCCTCGCCTGGC | |
| | CACTCAGCCTTAGTTGTCTCTGTTGTCTTATAGAGGTCTACTTGAAG | |
| | AAGGAAAAACAGGGGGCATGGTTTGACTGTCCTGTGAGCCCTTCTT | |
| | CCCTGCCTCCCCCACTCACAGTGACCCGGAATCCCTCGACATGGCA | |
| | GTCTAGATCATTCTTGAAGACGAAAGGGCCTCGTGATACGCCTATT | |
| | TTTATAGGTTAATGTCATGATAATAATGGTTTCTTAGACGTCAGGT | |
| | GGCACTTTTCGGGGAAATGTGCGCGGAACCCCTATTTGTTTATTTT | |
| | TCTAAATACATTCAAATATGTATCCGCTCATGAGACAATAACCCTG | |
| | ATAAATGCTTCAATAATATTGAAAAAGGAAGAGTATGAGTATTCA | |
| | ACATTTCCGTGTCGCCCTTATTCCCTTTTTTGCGGCATTTTGCCTTC | |
| | CTGTTTTTGCTCACCCAGAAACGCTGGTGAAAGTAAAAGATGCTGA | |
| | AGATCAGTTGGGTGCACGAGTGGGTTACATCGAACTGGATCTCAA | |
| | CAGCGGTAAGATCCTTGAGAGTTTTCGCCCCGAAGAACGTTTTCCA | |
| | ATGATGAGCACTTTTAAAGTTCTGCTATGTGGCGCGGTATTATCCC | |
| | GTATTGACGCCGGGCAAGAGCAACTCGGTCGCCGCATACACTATT | |
| | CTCAGAATGACTTGGTTGAGTACTCACCAGTCACAGAAAAGCATCT | |
| | TACGGATGGCATGACAGTAAGAGAATTATGCAGTGCTGCCATAAC | |
| | CATGAGTGATAACACTGCGGCCAACTTACTTCTGACAACGATCGG | |
| | AGGACCGAAGGAGCTAACCGCTTTTTTGCACAACATGGGGGATCA | |
| | TGTAACTCGCCTTGATCGTTGGGAACCGGAGCTGAATGAAGCCAT | |
| | ACCAAACGACGAGCGTGACACCACGATGCCTGTAGCAATGGCAAC | |
| | AACGTTGCGCAAACTATTAACTGGCGAACTACTTACTCTAGCTTCC | |
| | CGGCAACAATTAATAGACTGGATGGAGGCGGATAAAGTTGCAGGA | |
| | CCACTTCTGCGCTCGGCCCTTCCGGCTGGCTGGTTTATTGCTGATA | |
| | AATCTGGAGCCGGTGAGCGTGGGTCTCGCGGTATCATTGCAGCACT | |
| | GGGGCCAGATGGTAAGCCCTCCCGTATCGTAGTTATCTACACGACG | |
| | GGGAGTCAGGCAACTATGGATGAACGAAATAGACAGATCGCTGAG | |
| | ATAGGTGCCTCACTGATTAAGCATTGGTAACTGTCAGACCAAGTTT | |
| | ACTCATATATACTTTAGATTGATTTAAAACTTCATTTTTAATTTAAA | |
| | AGGATCTAGGTGAAGATCCTTTTTGATAATCTCATGACCAAAATCC | |
| | CTTAACGTGAGTTTTCGTTCCACTGAGCGTCAGACCCCGTAGAAAA | |
| | GATCAAAGGATCTTCTTGAGATCCTTTTTTTCTGCGCGTAATCTGCT | |
| | GCTTGCAAACAAAAAAACCACCGCTACCAGCGGTGGTTTGTTTGCC | |
| | GGATCAAGAGCTACCAACTCTTTTTCCGAAGGTAACTGGCTTCAGC | |
| | AGAGCGCAGATACCAAATACTGTTCTTCTAGTGTAGCCGTAGTTAG | |
| | GCCACCACTTCAAGAACTCTGTAGCACCGCCTACATACCTCGCTCT | |
| | GCTAATCCTGTTACCAGTGGCTGCTGCCAGTGGCGATAAGTCGTGT | |
| | CTTACCGGGTTGGACTCAAGACGATAGTTACCGGATAAGGCGCAG | |
| | CGGTCGGGCTGAACGGGGGGTTCGTGCACACAGCCCAGCTTGGAG | |
| | CGAACGACCTACACCGAACTGAGATACCTACAGCGTGAGCTATGA | |
| | GAAAGCGCCACGCTTCCCGAAGGGAGAAAGGCGGACAGGTATCCG | |
| | GTAAGCGGCAGGGTCGGAACAGGAGAGCGCACGAGGGAGCTTCC | |
| | AGGGGGAAACGCCTGGTATCTTTATAGTCCTGTCGGGTTTCGCCAC | |
| | CTCTGACTTGAGCGTCGATTTTTGTGATGCTCGTCAGGGGGGCGGA | |
| | GCCTATGGAAAAACGCCAGCAACGGATGCGCCGCGTGCGGCTGCT | |
| | GGAGATGGCGGACGCGATGGATATGTTCTGCCAAGGGTTGGTTTG | |
| | CGCATTCACAGTTCTCCGCAAGAATTGATTGGCTCCAATTCTTGGA | |
| | GTGGTGAATCCGTTAGCGAGGTGCCGCCGGCTTCCATTCAGGTCGA | |
| | GGTGGCCCGGCTCCATGCACCGCGACGCAACGCGGGGAGGCAGAC | |
| | AAGGTATAGGGCGGCGCCTACAATCCATGCCAACCCGTTCCATGT | |
| | GCTCGCCGAGGCGGCATAAATCCCCGTGACGATCAGCGGTCCAAT | |
| | GATCGAAGTTAGGCTGGTAAGAGCCGCGAGCGATCCTTGAAGCTG | |
| | TCCCTGATGGTCGTCATCTACCTGCCTGGACAGCATGGCCTGCAAC | |
| | GCGGGCATCCCGATGCCGCCGGAAGCGAGAAGAATCATAATGGGG | |
| | AAGGCCATCCAGCCTCGCGTCGGGGAGCTTTTTGCAAAAGCCTAG | |
| | GCCTCCAAAAAAGCCTCCTCACTACTTCTGGAATAGCTCAGAGGCC | |
| | GAGGCGGCCTCGGCCTCTGCATAAATAAAAAAAATTAGTCAGCCA | |
| | TG | |
| 61 | AGCTTGGCCCATTGCATACGTTGTATCCATATCATAATATGTACAT | Farnesyl, c- |
| | TTATATTGGCTCATGTCCAACATTACCGCCATGTTGACATTGATTAT | term, from |
| | TGACTAGTTATTAATAGTAATCAATTACGGGGTCATTAGTTCATAG | HRAS |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGCCAGCAAT | |
| | TTTACGCAATTCGTGCTCGTGGACAACGGCGGCACGGGCGACGTG | |
| | ACCGTGGCCCCCAGCAACTTCGCCAATGGCATCGCCGAATGGATC | |
| | AGCAGCAACAGCAGGAGCCAGGCGTATAAAGTTACGTGCAGCGTC | |
| | AGACAGAGCAGCGCCCAGAACAGGAAATATACGATCAAGGTCGA | |
| | GGTTCCCAAGGGAGCTTGGAGGAGCTATCTTAATATGGAGCTGAC | |
| | CATCCCCATCTTCGCGACAAATTCAGACTGCGAGCTCATCGTGAAG | |
| | GCAATGCAGGGCCTCTTGAAAGATGGCAACCCCATCCCAAGCGCA | |
| | ATCGCGGCCAACTCAGGAATCTACTCCCAGAACTACCCCATCGTGC | |
| | AGAAGCTGAACCCTCCTGATGAGAGTGGCCCCGGCTGCATGAGCT | |
| | GCAAGTGTGTGCTCTCCTGATAAGGATCCAAGCTTATCGATACCGT | |
| | CGACCTCGAGGGCCCAGATCTAATTCACCCCACCAGTGCAGGCTG | |
| | CCTATCAGAAAGTGGTGGCTGGTGTGGCTAATGCCCTGGCCCACA | |
| | AGTATCACTAAGCTCGCTTTCTTGCTGTCCAATTTCTATTAAAGGTT | |
| | CCTTTGTTCCCTAAGTCCAACTACTAAACTGGGGGATATTATGAAG | |
| | GGCCTTGAGCATCTGGATTCTGCCTAATAAAAAACATTTATTTTCA | |
| | TTGCAATGATGTATTTAAATTATTTCTGAATATTTTACTAAAAAGG | |
| | GAATGTGGGAGGTCAGTGCATTTAAAACATAAAGAAATGAAGAGC | |
| | TAGTTCAAACCTTGGGAAAATACACTATATCTTAAACTCCATGAAA | |
| | GAAGGTGAGGCTGCAAACAGCTAATGCACATTGGCAACAGCCCCT | |
| | GATGCCTATGCCTTATTCATCCCTCAGAAAAGGATTCAAGTAGAGG | |
| | CTTGATTTGGAGGTTAAAGTTTTGCTATGCTGTATTTTACATTACTT | |
| | ATTGTTTTAGCTGTCCTCATGAATGTCTTTTCACTACCCATTTGCTT | |
| | ATCCTGCATCTCTCAGCCTTGACTCCACTCAGTTCTCTTGCTTAGAG | |
| | ATACCACCTTTCCCCTGAAGTGTTCCTTCCATGTTTTACGGCGAGAT | |
| | GGTTTCTCCTCGCCTGGCCACTCAGCCTTAGTTGTCTCTGTTGTCTT | |
| | ATAGAGGTCTACTTGAAGAAGGAAAAACAGGGGGCATGGTTTGAC | |
| | TGTCCTGTGAGCCCTTCTTCCCTGCCTCCCCCACTCACAGTGACCCG | |
| | GAATCCCTCGACATGGCAGTCTAGATCATTCTTGAAGACGAAAGG | |
| | GCCTCGTGATACGCCTATTTTTATAGGTTAATGTCATGATAATAAT | |
| | GGTTTCTTAGACGTCAGGTGGCACTTTTCGGGGAAATGTGCGCGGA | |
| | ACCCCTATTTGTTTATTTTTCTAAATACATTCAAATATGTATCCGCT | |
| | CATGAGACAATAACCCTGATAAATGCTTCAATAATATTGAAAAAG | |
| | GAAGAGTATGAGTATTCAACATTTCCGTGTCGCCCTTATTCCCTTTT | |
| | TTGCGGCATTTTGCCTTCCTGTTTTTGCTCACCCAGAAACGCTGGTG | |
| | AAAGTAAAAGATGCTGAAGATCAGTTGGGTGCACGAGTGGGTTAC | |
| | ATCGAACTGGATCTCAACAGCGGTAAGATCCTTGAGAGTTTTCGCC | |
| | CCGAAGAACGTTTTCCAATGATGAGCACTTTTAAAGTTCTGCTATG | |
| | TGGCGCGGTATTATCCCGTATTGACGCCGGGCAAGAGCAACTCGG | |
| | TCGCCGCATACACTATTCTCAGAATGACTTGGTTGAGTACTCACCA | |
| | GTCACAGAAAAGCATCTTACGGATGGCATGACAGTAAGAGAATTA | |
| | TGCAGTGCTGCCATAACCATGAGTGATAACACTGCGGCCAACTTAC | |
| | TTCTGACAACGATCGGAGGACCGAAGGAGCTAACCGCTTTTTTGCA | |
| | CAACATGGGGGATCATGTAACTCGCCTTGATCGTTGGGAACCGGA | |
| | GCTGAATGAAGCCATACCAAACGACGAGCGTGACACCACGATGCC | |
| | TGTAGCAATGGCAACAACGTTGCGCAAACTATTAACTGGCGAACT | |
| | ACTTACTCTAGCTTCCCGGCAACAATTAATAGACTGGATGGAGGCG | |
| | GATAAAGTTGCAGGACCACTTCTGCGCTCGGCCCTTCCGGCTGGCT | |
| | GGTTTATTGCTGATAAATCTGGAGCCGGTGAGCGTGGGTCTCGCGG | |
| | TATCATTGCAGCACTGGGGCCAGATGGTAAGCCCTCCCGTATCGTA | |
| | GTTATCTACACGACGGGGAGTCAGGCAACTATGGATGAACGAAAT | |
| | AGACAGATCGCTGAGATAGGTGCCTCACTGATTAAGCATTGGTAA | |
| | CTGTCAGACCAAGTTTACTCATATATACTTTAGATTGATTTAAAAC | |
| | TTCATTTTTAATTTAAAAGGATCTAGGTGAAGATCCTTTTTGATAAT | |
| | CTCATGACCAAAATCCCTTAACGTGAGTTTTCGTTCCACTGAGCGT | |
| | CAGACCCCGTAGAAAAGATCAAAGGATCTTCTTGAGATCCTTTTTT | |
| | TCTGCGCGTAATCTGCTGCTTGCAAACAAAAAAACCACCGCTACCA | |
| | GCGGTGGTTTGTTTGCCGGATCAAGAGCTACCAACTCTTTTTCCGA | |
| | AGGTAACTGGCTTCAGCAGAGCGCAGATACCAAATACTGTTCTTCT | |
| | AGTGTAGCCGTAGTTAGGCCACCACTTCAAGAACTCTGTAGCACCG | |
| | CCTACATACCTCGCTCTGCTAATCCTGTTACCAGTGGCTGCTGCCA | |
| | GTGGCGATAAGTCGTGTCTTACCGGGTTGGACTCAAGACGATAGTT | |
| | ACCGGATAAGGCGCAGCGGTCGGGCTGAACGGGGGGTTCGTGCAC | |
| | ACAGCCCAGCTTGGAGCGAACGACCTACACCGAACTGAGATACCT | |
| | ACAGCGTGAGCTATGAGAAAGCGCCACGCTTCCCGAAGGGAGAAA | |
| | GGCGGACAGGTATCCGGTAAGCGGCAGGGTCGGAACAGGAGAGC | |
| | GCACGAGGGAGCTTCCAGGGGGAAACGCCTGGTATCTTTATAGTC | |
| | CTGTCGGGTTTCGCCACCTCTGACTTGAGCGTCGATTTTTGTGATGC | |
| | TCGTCAGGGGGGCGGAGCCTATGGAAAAACGCCAGCAACGGATGC | |
| | GCCGCGTGCGGCTGCTGGAGATGGCGGACGCGATGGATATGTTCT | |
| | GCCAAGGGTTGGTTTGCGCATTCACAGTTCTCCGCAAGAATTGATT | |
| | GGCTCCAATTCTTGGAGTGGTGAATCCGTTAGCGAGGTGCCGCCGG | |
| | CTTCCATTCAGGTCGAGGTGGCCCGGCTCCATGCACCGCGACGCAA | |
| | CGCGGGGAGGCAGACAAGGTATAGGGCGGCGCCTACAATCCATGC | |
| | CAACCCGTTCCATGTGCTCGCCGAGGCGGCATAAATCCCCGTGACG | |
| | ATCAGCGGTCCAATGATCGAAGTTAGGCTGGTAAGAGCCGCGAGC | |
| | GATCCTTGAAGCTGTCCCTGATGGTCGTCATCTACCTGCCTGGACA | |
| | GCATGGCCTGCAACGCGGGCATCCCGATGCCGCCGGAAGCGAGAA | |
| | GAATCATAATGGGGAAGGCCATCCAGCCTCGCGTCGGGGAGCTTT | |
| | TTGCAAAAGCCTAGGCCTCCAAAAAAGCCTCCTCACTACTTCTGGA | |
| | ATAGCTCAGAGGCCGAGGCGGCCTCGGCCTCTGCATAAATAAAAA | |
| | AAATTAGTCAGCCATG | |
| 62 | AGCTTGGCCCATTGCATACGTTGTATCCATATCATAATATGTACAT | HIV MA (N- |
| | TTATATTGGCTCATGTCCAACATTACCGCCATGTTGACATTGATTAT | term) |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCCGC | |
| | GCCTCCGTGCTGTCCGGCGGCGAGCTGGACAAGTGGGAGAAGATC | |
| | CGCCTGCGCCCCGGCGGCAAGAAGCAGTACAAGCTGAAGCACATC | |
| | GTGTGGGCCTCCCGCGAGCTGGAGCGCTTCGCCGTGAACCCCGGC | |
| | CTGCTGGAAACCTCCGAGGGCTGCCGCCAGATCCTGGGCCAGCTG | |
| | CAGCCCTCCCTGCAAACCGGCTCCGAGGAGCTGCGCTCCCTGTACA | |
| | ACACCATCGCCGTGCTGTACTGCGTGCACCAGCGCATCGACGTGA | |
| | AGGACACCAAGGAGGCCCTGGACAAGATCGAGGAGGAGCAGAAC | |
| | AAGTCCAAGAAGAAGGCCCAGCAGGCCGCCGCCGACACCGGCAA | |
| | CAACTCCCAGGTGTCCCAGAACTACCCCATCGTGCAGATGGCCAG | |
| | CAATTTTACGCAATTCGTGCTCGTGGACAACGGCGGCACGGGCGA | |
| | CGTGACCGTGGCCCCCAGCAACTTCGCCAATGGCATCGCCGAATG | |
| | GATCAGCAGCAACAGCAGGAGCCAGGCGTATAAAGTTACGTGCAG | |
| | CGTCAGACAGAGCAGCGCCCAGAACAGGAAATATACGATCAAGGT | |
| | CGAGGTTCCCAAGGGAGCTTGGAGGAGCTATCTTAATATGGAGCT | |
| | GACCATCCCCATCTTCGCGACAAATTCAGACTGCGAGCTCATCGTG | |
| | AAGGCAATGCAGGGCCTCTTGAAAGATGGCAACCCCATCCCAAGC | |
| | GCAATCGCGGCCAACTCAGGAATCTACTAAGGATCCAAGCTTATC | |
| | GATACCGTCGACCTCGAGGGCCCAGATCTAATTCACCCCACCAGTG | |
| | CAGGCTGCCTATCAGAAAGTGGTGGCTGGTGTGGCTAATGCCCTG | |
| | GCCCACAAGTATCACTAAGCTCGCTTTCTTGCTGTCCAATTTCTATT | |
| | AAAGGTTCCTTTGTTCCCTAAGTCCAACTACTAAACTGGGGGATAT | |
| | TATGAAGGGCCTTGAGCATCTGGATTCTGCCTAATAAAAAACATTT | |
| | ATTTTCATTGCAATGATGTATTTAAATTATTTCTGAATATTTTACTA | |
| | AAAAGGGAATGTGGGAGGTCAGTGCATTTAAAACATAAAGAAATG | |
| | AAGAGCTAGTTCAAACCTTGGGAAAATACACTATATCTTAAACTCC | |
| | ATGAAAGAAGGTGAGGCTGCAAACAGCTAATGCACATTGGCAACA | |
| | GCCCCTGATGCCTATGCCTTATTCATCCCTCAGAAAAGGATTCAAG | |
| | TAGAGGCTTGATTTGGAGGTTAAAGTTTTGCTATGCTGTATTTTAC | |
| | ATTACTTATTGTTTTAGCTGTCCTCATGAATGTCTTTTCACTACCCA | |
| | TTTGCTTATCCTGCATCTCTCAGCCTTGACTCCACTCAGTTCTCTTG | |
| | CTTAGAGATACCACCTTTCCCCTGAAGTGTTCCTTCCATGTTTTACG | |
| | GCGAGATGGTTTCTCCTCGCCTGGCCACTCAGCCTTAGTTGTCTCT | |
| | GTTGTCTTATAGAGGTCTACTTGAAGAAGGAAAAACAGGGGGCAT | |
| | GGTTTGACTGTCCTGTGAGCCCTTCTTCCCTGCCTCCCCCACTCACA | |
| | GTGACCCGGAATCCCTCGACATGGCAGTCTAGATCATTCTTGAAGA | |
| | CGAAAGGGCCTCGTGATACGCCTATTTTTATAGGTTAATGTCATGA | |
| | TAATAATGGTTTCTTAGACGTCAGGTGGCACTTTTCGGGGAAATGT | |
| | GCGCGGAACCCCTATTTGTTTATTTTTCTAAATACATTCAAATATGT | |
| | ATCCGCTCATGAGACAATAACCCTGATAAATGCTTCAATAATATTG | |
| | AAAAAGGAAGAGTATGAGTATTCAACATTTCCGTGTCGCCCTTATT | |
| | CCCTTTTTTGCGGCATTTTGCCTTCCTGTTTTTGCTCACCCAGAAAC | |
| | GCTGGTGAAAGTAAAAGATGCTGAAGATCAGTTGGGTGCACGAGT | |
| | GGGTTACATCGAACTGGATCTCAACAGCGGTAAGATCCTTGAGAG | |
| | TTTTCGCCCCGAAGAACGTTTTCCAATGATGAGCACTTTTAAAGTT | |
| | CTGCTATGTGGCGCGGTATTATCCCGTATTGACGCCGGGCAAGAGC | |
| | AACTCGGTCGCCGCATACACTATTCTCAGAATGACTTGGTTGAGTA | |
| | CTCACCAGTCACAGAAAAGCATCTTACGGATGGCATGACAGTAAG | |
| | AGAATTATGCAGTGCTGCCATAACCATGAGTGATAACACTGCGGC | |
| | CAACTTACTTCTGACAACGATCGGAGGACCGAAGGAGCTAACCGC | |
| | TTTTTTGCACAACATGGGGGATCATGTAACTCGCCTTGATCGTTGG | |
| | GAACCGGAGCTGAATGAAGCCATACCAAACGACGAGCGTGACACC | |
| | ACGATGCCTGTAGCAATGGCAACAACGTTGCGCAAACTATTAACT | |
| | GGCGAACTACTTACTCTAGCTTCCCGGCAACAATTAATAGACTGGA | |
| | TGGAGGCGGATAAAGTTGCAGGACCACTTCTGCGCTCGGCCCTTCC | |
| | GGCTGGCTGGTTTATTGCTGATAAATCTGGAGCCGGTGAGCGTGGG | |
| | TCTCGCGGTATCATTGCAGCACTGGGGCCAGATGGTAAGCCCTCCC | |
| | GTATCGTAGTTATCTACACGACGGGGAGTCAGGCAACTATGGATG | |
| | AACGAAATAGACAGATCGCTGAGATAGGTGCCTCACTGATTAAGC | |
| | ATTGGTAACTGTCAGACCAAGTTTACTCATATATACTTTAGATTGA | |
| | TTTAAAACTTCATTTTTAATTTAAAAGGATCTAGGTGAAGATCCTTT | |
| | TTGATAATCTCATGACCAAAATCCCTTAACGTGAGTTTTCGTTCCA | |
| | CTGAGCGTCAGACCCCGTAGAAAAGATCAAAGGATCTTCTTGAGA | |
| | TCCTTTTTTTCTGCGCGTAATCTGCTGCTTGCAAACAAAAAAACCA | |
| | CCGCTACCAGCGGTGGTTTGTTTGCCGGATCAAGAGCTACCAACTC | |
| | TTTTTCCGAAGGTAACTGGCTTCAGCAGAGCGCAGATACCAAATAC | |
| | TGTTCTTCTAGTGTAGCCGTAGTTAGGCCACCACTTCAAGAACTCT | |
| | GTAGCACCGCCTACATACCTCGCTCTGCTAATCCTGTTACCAGTGG | |
| | CTGCTGCCAGTGGCGATAAGTCGTGTCTTACCGGGTTGGACTCAAG | |
| | ACGATAGTTACCGGATAAGGCGCAGCGGTCGGGCTGAACGGGGGG | |
| | TTCGTGCACACAGCCCAGCTTGGAGCGAACGACCTACACCGAACT | |
| | GAGATACCTACAGCGTGAGCTATGAGAAAGCGCCACGCTTCCCGA | |
| | AGGGAGAAAGGCGGACAGGTATCCGGTAAGCGGCAGGGTCGGAA | |
| | CAGGAGAGCGCACGAGGGAGCTTCCAGGGGGAAACGCCTGGTATC | |
| | TTTATAGTCCTGTCGGGTTTCGCCACCTCTGACTTGAGCGTCGATTT | |
| | TTGTGATGCTCGTCAGGGGGGCGGAGCCTATGGAAAAACGCCAGC | |
| | AACGGATGCGCCGCGTGCGGCTGCTGGAGATGGCGGACGCGATGG | |
| | ATATGTTCTGCCAAGGGTTGGTTTGCGCATTCACAGTTCTCCGCAA | |
| | GAATTGATTGGCTCCAATTCTTGGAGTGGTGAATCCGTTAGCGAGG | |
| | TGCCGCCGGCTTCCATTCAGGTCGAGGTGGCCCGGCTCCATGCACC | |
| | GCGACGCAACGCGGGGAGGCAGACAAGGTATAGGGCGGCGCCTA | |
| | CAATCCATGCCAACCCGTTCCATGTGCTCGCCGAGGCGGCATAAAT | |
| | CCCCGTGACGATCAGCGGTCCAATGATCGAAGTTAGGCTGGTAAG | |
| | AGCCGCGAGCGATCCTTGAAGCTGTCCCTGATGGTCGTCATCTACC | |
| | TGCCTGGACAGCATGGCCTGCAACGCGGGCATCCCGATGCCGCCG | |
| | GAAGCGAGAAGAATCATAATGGGGAAGGCCATCCAGCCTCGCGTC | |
| | GGGGAGCTTTTTGCAAAAGCCTAGGCCTCCAAAAAAGCCTCCTCA | |
| | CTACTTCTGGAATAGCTCAGAGGCCGAGGCGGCCTCGGCCTCTGCA | |
| | TAAATAAAAAAAATTAGTCAGCCATGAGCTTGGCCCATTGCATAC | |
| | GTTGTATCCATATCATAATATGTACATTTATATTGGCTCATGTCCAA | |
| | CATTACCGCCATGTTGACATTGATTATTGACTAGTTATTAATAGTA | |
| | ATCAATTACGGGGTCATTAGTTCATAGCCCATATATGGAGTTCCGC | |
| | GTTACATAACTTACGGTAAATGGCCCGCCTGGCTGACCGCCCAACG | |
| | ACCCCCGCCCATTGACGTCAATAATGACGTATGTTCCCATAGTAAC | |
| | GCCAATAGGGACTTTCCATTGACGTCAATGGGTGGAGTATTTACGG | |
| | TAAACTGCCCACTTGGCAGTACATCAAGTGTATCATATGCCAAGTA | |
| | CGCCCCCTATTGACGTCAATGACGGTAAATGGCCCGCCTGGCATTA | |
| | TGCCCAGTACATGACCTTATGGGACTTTCCTACTTGGCAGTACATC | |
| | TACGTATTAGTCATCGCTATTACCATGGTGATGCGGTTTTGGCAGT | |
| | ACATCAATGGGCGTGGATAGCGGTTTGACTCACGGGGATTTCCAA | |
| | GTCTCCACCCCATTGACGTCAATGGGAGTTTGTTTTGGCACCAAAA | |
| | TCAACGGGACTTTCCAAAATGTCGTAACAACTCCGCCCCATTGACG | |
| | CAAATGGGCGGTAGGCGTGTACGGTGGGAGGTCTATATAAGCAGA | |
| | GCTCGTTTAGTGAACCGTCAGATCGCCTGGAGACGCCATCCACGCT | |
| | GTTTTGACCTCCATAGAAGACACCGGGACCGATCCAGCCTCCCCTC | |
| | GAAGCTGATCCTGAGAACTTCAGGGTGAGTCTATGGGACCCTTGAT | |
| | GTTTTCTTTCCCCTTCTTTTCTATGGTTAAGTTCATGTCATAGGAAG | |
| | GGGAGAAGTAACAGGGTACACATATTGACCAAATCAGGGTAATTT | |
| | TGCATTTGTAATTTTAAAAAATGCTTTCTTCTTTTAATATACTTTTTT | |
| | GTTTATCTTATTTCTAATACTTTCCCTAATCTCTTTCTTTCAGGGCA | |
| | ATAATGATACAATGTATCATGCCTCTTTGCACCATTCTAAAGAATA | |
| | ACAGTGATAATTTCTGGGTTAAGGCAATAGCAATATTTCTGCATAT | |
| | AAATATTTCTGCATATAAATTGTAACTGATGTAAGAGGTTTCATAT | |
| | TGCTAATAGCAGCTACAATCCAGCTACCATTCTGCTTTTATTTTATG | |
| | GTTGGGATAAGGCTGGATTATTCTGAGTCCAAGCTAGGCCCTTTTG | |
| | CTAATCATGTTCATACCTCTTATCTTCCTCCCACAGCTCCTGGGCAA | |
| | CGTGCTGGTCTGTGTGCTGGCCCATCACTTTGGCAAAGAATTCCGC | |
| | GGGCGGCCGCCACCATGGACAGCGGGAGGGATTTCCTCACGCTGC | |
| | ACGGTTTGCAGGACGACGAGGACCTCCAAGCTTTGCTCAAAGGCA | |
| | GTCAACTTCTCAAGGTCAAGAGCAGCTCCTGGAGGCGCGAACGGT | |
| | TTTACAAGTTGCAAGAAGATTGCAAAACAATTTGGCAAGAGAGCC | |
| | GGAAAGTCATGAGAACTCCCGAGAGCCAACTGTTCAGCATCGAGG | |
| | ACATCCAAGAAGTCAGGATGGGCCATAGGACCGAGGGCTTGGAAA | |
| | AATTCGCCAGGGACGTGCCCGAAGACCGATGCTTTAGCATCGTGTT | |
| | CAAAGATCAGAGGAACACGTTGGACTTGATCGCCCCCAGTCCGGC | |
| | TGATGCCCAGCATTGGGTTCTGGGGCTCCATAAGATCATCCATCAT | |
| | AGCGGCAGCATGGACCAGAGGCAGAAACTCCAACATTGGATTCAT | |
| | AGTTGTCTTAGGAAAGCCGACAAGAACAAGGACAACAAGATGAGC | |
| | TTCAAGGAGTTGCAGAATTTTCTTAAAGAGCTGAATATCCAGTCCC | |
| | AGAACTACCCCATCGTGCAGATGTCCAATTTACTGACCGTACACCA | |
| | AAATTTGCCTGCATTACCGGTCGATGCAACGAGTGATGAGGTTCGC | |
| | AAGAACCTGATGGACATGTTCAGGGATCGCCAGGCGTTTTCTGAG | |
| | CATACCTGGAAAATGCTTCTGTCCGTTTGCCGGTCGTGGGCGGCAT | |
| | GGTGCAAGTTGAATAACCGGAAATGGTTTCCCGCAGAACCTGAAG | |
| | ATGTTCGCGATTATCTTCTATATCTTCAGGCGCGCGGTCTGGCAGT | |
| | AAAAACTATCCAGCAACATTTGGGCCAGCTAAACATGCTTCATCGT | |
| | CGGTCCGGGCTGCCACGACCAAGTGACAGCAATGCTGTTTCACTG | |
| | GTTATGCGGCGGATCCGAAAAGAAAACGTTGATGCCGGTGAACGT | |
| | GCAAAACAGGCTCTAGCGTTCGAACGCACTGATTTCGACCAGGTTC | |
| | GTTCACTCATGGAAAATAGCGATCGCTGCCAGGATATACGTAATCT | |
| | GGCATTTCTGGGGATTGCTTATAACACCCTGTTACGTATAGCCGAA | |
| | ATTGCCAGGATCAGGGTTAAAGATATCTCACGTACTGACGGTGGG | |
| | AGAATGTTAATCCATATTGGCAGAACGAAAACGCTGGTTAGCACC | |
| | GCAGGTGTAGAGAAGGCACTTAGCCTGGGGGTAACTAAACTGGTC | |
| | GAGCGATGGATTTCCGTCTCTGGTGTAGCTGATGATCCGAATAACT | |
| | ACCTGTTTTGCCGGGTCAGAAAAAATGGTGTTGCCGCGCCATCTGC | |
| | CACCAGCCAGCTATCAACTCGCGCCCTGGAAGGGATTTTTGAAGC | |
| | AACTCATCGATTGATTTACGGCGCTAAGGATGACTCTGGTCAGAGA | |
| | TACCTGGCCTGGTCTGGACACAGTGCCCGTGTCGGAGCCGCGCGA | |
| | GATATGGCCCGCGCTGGAGTTTCAATACCGGAGATCATGCAAGCT | |
| | GGTGGCTGGACCAATGTAAATATTGTCATGAACTATATCCGTAACC | |
| | TGGATAGTGAAACAGGGGCAATGGTGCGCCTGCTGGAAGATGGCG | |
| | ATTAAGGATCCAAGCTTATCGATACCGTCGACCTCGAGGGCCCAG | |
| | ATCTAATTCACCCCACCAGTGCAGGCTGCCTATCAGAAAGTGGTGG | |
| | CTGGTGTGGCTAATGCCCTGGCCCACAAGTATCACTAAGCTCGCTT | |
| | TCTTGCTGTCCAATTTCTATTAAAGGTTCCTTTGTTCCCTAAGTCCA | |
| | ACTACTAAACTGGGGGATATTATGAAGGGCCTTGAGCATCTGGATT | |
| | CTGCCTAATAAAAAACATTTATTTTCATTGCAATGATGTATTTAAA | |
| | TTATTTCTGAATATTTTACTAAAAAGGGAATGTGGGAGGTCAGTGC | |
| | ATTTAAAACATAAAGAAATGAAGAGCTAGTTCAAACCTTGGGAAA | |
| | ATACACTATATCTTAAACTCCATGAAAGAAGGTGAGGCTGCAAAC | |
| | AGCTAATGCACATTGGCAACAGCCCCTGATGCCTATGCCTTATTCA | |
| | TCCCTCAGAAAAGGATTCAAGTAGAGGCTTGATTTGGAGGTTAAA | |
| | GTTTTGCTATGCTGTATTTTACATTACTTATTGTTTTAGCTGTCCTCA | |
| | TGAATGTCTTTTCACTACCCATTTGCTTATCCTGCATCTCTCAGCCT | |
| | TGACTCCACTCAGTTCTCTTGCTTAGAGATACCACCTTTCCCCTGAA | |
| | GTGTTCCTTCCATGTTTTACGGCGAGATGGTTTCTCCTCGCCTGGCC | |
| | ACTCAGCCTTAGTTGTCTCTGTTGTCTTATAGAGGTCTACTTGAAG | |
| | AAGGAAAAACAGGGGGCATGGTTTGACTGTCCTGTGAGCCCTTCTT | |
| | CCCTGCCTCCCCCACTCACAGTGACCCGGAATCCCTCGACATGGCA | |
| | GTCTAGATCATTCTTGAAGACGAAAGGGCCTCGTGATACGCCTATT | |
| | TTTATAGGTTAATGTCATGATAATAATGGTTTCTTAGACGTCAGGT | |
| | GGCACTTTTCGGGGAAATGTGCGCGGAACCCCTATTTGTTTATTTT | |
| | TCTAAATACATTCAAATATGTATCCGCTCATGAGACAATAACCCTG | |
| | ATAAATGCTTCAATAATATTGAAAAAGGAAGAGTATGAGTATTCA | |
| | ACATTTCCGTGTCGCCCTTATTCCCTTTTTTGCGGCATTTTGCCTTC | |
| | CTGTTTTTGCTCACCCAGAAACGCTGGTGAAAGTAAAAGATGCTGA | |
| | AGATCAGTTGGGTGCACGAGTGGGTTACATCGAACTGGATCTCAA | |
| | CAGCGGTAAGATCCTTGAGAGTTTTCGCCCCGAAGAACGTTTTCCA | |
| | ATGATGAGCACTTTTAAAGTTCTGCTATGTGGCGCGGTATTATCCC | |
| | GTATTGACGCCGGGCAAGAGCAACTCGGTCGCCGCATACACTATT | |
| | CTCAGAATGACTTGGTTGAGTACTCACCAGTCACAGAAAAGCATCT | |
| | TACGGATGGCATGACAGTAAGAGAATTATGCAGTGCTGCCATAAC | |
| | CATGAGTGATAACACTGCGGCCAACTTACTTCTGACAACGATCGG | |
| | AGGACCGAAGGAGCTAACCGCTTTTTTGCACAACATGGGGGATCA | |
| | TGTAACTCGCCTTGATCGTTGGGAACCGGAGCTGAATGAAGCCAT | |
| | ACCAAACGACGAGCGTGACACCACGATGCCTGTAGCAATGGCAAC | |
| | AACGTTGCGCAAACTATTAACTGGCGAACTACTTACTCTAGCTTCC | |
| | CGGCAACAATTAATAGACTGGATGGAGGCGGATAAAGTTGCAGGA | |
| | CCACTTCTGCGCTCGGCCCTTCCGGCTGGCTGGTTTATTGCTGATA | |
| | AATCTGGAGCCGGTGAGCGTGGGTCTCGCGGTATCATTGCAGCACT | |
| | GGGGCCAGATGGTAAGCCCTCCCGTATCGTAGTTATCTACACGACG | |
| | GGGAGTCAGGCAACTATGGATGAACGAAATAGACAGATCGCTGAG | |
| | ATAGGTGCCTCACTGATTAAGCATTGGTAACTGTCAGACCAAGTTT | |
| | ACTCATATATACTTTAGATTGATTTAAAACTTCATTTTTAATTTAAA | |
| | AGGATCTAGGTGAAGATCCTTTTTGATAATCTCATGACCAAAATCC | |
| | CTTAACGTGAGTTTTCGTTCCACTGAGCGTCAGACCCCGTAGAAAA | |
| | GATCAAAGGATCTTCTTGAGATCCTTTTTTTCTGCGCGTAATCTGCT | |
| | GCTTGCAAACAAAAAAACCACCGCTACCAGCGGTGGTTTGTTTGCC | |
| | GGATCAAGAGCTACCAACTCTTTTTCCGAAGGTAACTGGCTTCAGC | |
| | AGAGCGCAGATACCAAATACTGTTCTTCTAGTGTAGCCGTAGTTAG | |
| | GCCACCACTTCAAGAACTCTGTAGCACCGCCTACATACCTCGCTCT | |
| | GCTAATCCTGTTACCAGTGGCTGCTGCCAGTGGCGATAAGTCGTGT | |
| | CTTACCGGGTTGGACTCAAGACGATAGTTACCGGATAAGGCGCAG | |
| | CGGTCGGGCTGAACGGGGGGTTCGTGCACACAGCCCAGCTTGGAG | |
| | CGAACGACCTACACCGAACTGAGATACCTACAGCGTGAGCTATGA | |
| | GAAAGCGCCACGCTTCCCGAAGGGAGAAAGGCGGACAGGTATCCG | |
| | GTAAGCGGCAGGGTCGGAACAGGAGAGCGCACGAGGGAGCTTCC | |
| | AGGGGGAAACGCCTGGTATCTTTATAGTCCTGTCGGGTTTCGCCAC | |
| | CTCTGACTTGAGCGTCGATTTTTGTGATGCTCGTCAGGGGGGCGGA | |
| | GCCTATGGAAAAACGCCAGCAACGGATGCGCCGCGTGCGGCTGCT | |
| | GGAGATGGCGGACGCGATGGATATGTTCTGCCAAGGGTTGGTTTG | |
| | CGCATTCACAGTTCTCCGCAAGAATTGATTGGCTCCAATTCTTGGA | |
| | GTGGTGAATCCGTTAGCGAGGTGCCGCCGGCTTCCATTCAGGTCGA | |
| | GGTGGCCCGGCTCCATGCACCGCGACGCAACGCGGGGAGGCAGAC | |
| | AAGGTATAGGGCGGCGCCTACAATCCATGCCAACCCGTTCCATGT | |
| | GCTCGCCGAGGCGGCATAAATCCCCGTGACGATCAGCGGTCCAAT | |
| | GATCGAAGTTAGGCTGGTAAGAGCCGCGAGCGATCCTTGAAGCTG | |
| | TCCCTGATGGTCGTCATCTACCTGCCTGGACAGCATGGCCTGCAAC | |
| | GCGGGCATCCCGATGCCGCCGGAAGCGAGAAGAATCATAATGGGG | |
| | AAGGCCATCCAGCCTCGCGTCGGGGAGCTTTTTGCAAAAGCCTAG | |
| | GCCTCCAAAAAAGCCTCCTCACTACTTCTGGAATAGCTCAGAGGCC | |
| | GAGGCGGCCTCGGCCTCTGCATAAATAAAAAAAATTAGTCAGCCA | |
| | TG | |
| 63 | AGCTTGGCCCATTGCATACGTTGTATCCATATCATAATATGTACAT | N-term PH |
| | TTATATTGGCTCATGTCCAACATTACCGCCATGTTGACATTGATTAT | domain |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGACAGCGGG | |
| | AGGGATTTCCTCACGCTGCACGGTTTGCAGGACGACGAGGACCTC | |
| | CAAGCTTTGCTCAAAGGCAGTCAACTTCTCAAGGTCAAGAGCAGC | |
| | TCCTGGAGGCGCGAACGGTTTTACAAGTTGCAAGAAGATTGCAAA | |
| | ACAATTTGGCAAGAGAGCCGGAAAGTCATGAGAACTCCCGAGAGC | |
| | CAACTGTTCAGCATCGAGGACATCCAAGAAGTCAGGATGGGCCAT | |
| | AGGACCGAGGGCTTGGAAAAATTCGCCAGGGACGTGCCCGAAGAC | |
| | CGATGCTTTAGCATCGTGTTCAAAGATCAGAGGAACACGTTGGACT | |
| | TGATCGCCCCCAGTCCGGCTGATGCCCAGCATTGGGTTCTGGGGCT | |
| | CCATAAGATCATCCATCATAGCGGCAGCATGGACCAGAGGCAGAA | |
| | ACTCCAACATTGGATTCATAGTTGTCTTAGGAAAGCCGACAAGAA | |
| | CAAGGACAACAAGATGAGCTTCAAGGAGTTGCAGAATTTTCTTAA | |
| | AGAGCTGAATATCCAGTCCCAGAACTACCCCATCGTGCAGATGTCC | |
| | AATTTACTGACCGTACACCAAAATTTGCCTGCATTACCGGTCGATG | |
| | CAACGAGTGATGAGGTTCGCAAGAACCTGATGGACATGTTCAGGG | |
| | ATCGCCAGGCGTTTTCTGAGCATACCTGGAAAATGCTTCTGTCCGT | |
| | TTGCCGGTCGTGGGCGGCATGGTGCAAGTTGAATAACCGGAAATG | |
| | GTTTCCCGCAGAACCTGAAGATGTTCGCGATTATCTTCTATATCTTC | |
| | AGGCGCGCGGTCTGGCAGTAAAAACTATCCAGCAACATTTGGGCC | |
| | AGCTAAACATGCTTCATCGTCGGTCCGGGCTGCCACGACCAAGTG | |
| | ACAGCAATGCTGTTTCACTGGTTATGCGGCGGATCCGAAAAGAAA | |
| | ACGTTGATGCCGGTGAACGTGCAAAACAGGCTCTAGCGTTCGAAC | |
| | GCACTGATTTCGACCAGGTTCGTTCACTCATGGAAAATAGCGATCG | |
| | CTGCCAGGATATACGTAATCTGGCATTTCTGGGGATTGCTTATAAC | |
| | ACCCTGTTACGTATAGCCGAAATTGCCAGGATCAGGGTTAAAGAT | |
| | ATCTCACGTACTGACGGTGGGAGAATGTTAATCCATATTGGCAGA | |
| | ACGAAAACGCTGGTTAGCACCGCAGGTGTAGAGAAGGCACTTAGC | |
| | CTGGGGGTAACTAAACTGGTCGAGCGATGGATTTCCGTCTCTGGTG | |
| | TAGCTGATGATCCGAATAACTACCTGTTTTGCCGGGTCAGAAAAAA | |
| | TGGTGTTGCCGCGCCATCTGCCACCAGCCAGCTATCAACTCGCGCC | |
| | CTGGAAGGGATTTTTGAAGCAACTCATCGATTGATTTACGGCGCTA | |
| | AGGATGACTCTGGTCAGAGATACCTGGCCTGGTCTGGACACAGTG | |
| | CCCGTGTCGGAGCCGCGCGAGATATGGCCCGCGCTGGAGTTTCAA | |
| | TACCGGAGATCATGCAAGCTGGTGGCTGGACCAATGTAAATATTG | |
| | TCATGAACTATATCCGTAACCTGGATAGTGAAACAGGGGCAATGG | |
| | TGCGCCTGCTGGAAGATGGCGATTAAGGATCCAAGCTTATCGATA | |
| | CCGTCGACCTCGAGGGCCCAGATCTAATTCACCCCACCAGTGCAG | |
| | GCTGCCTATCAGAAAGTGGTGGCTGGTGTGGCTAATGCCCTGGCCC | |
| | ACAAGTATCACTAAGCTCGCTTTCTTGCTGTCCAATTTCTATTAAA | |
| | GGTTCCTTTGTTCCCTAAGTCCAACTACTAAACTGGGGGATATTAT | |
| | GAAGGGCCTTGAGCATCTGGATTCTGCCTAATAAAAAACATTTATT | |
| | TTCATTGCAATGATGTATTTAAATTATTTCTGAATATTTTACTAAAA | |
| | AGGGAATGTGGGAGGTCAGTGCATTTAAAACATAAAGAAATGAAG | |
| | AGCTAGTTCAAACCTTGGGAAAATACACTATATCTTAAACTCCATG | |
| | AAAGAAGGTGAGGCTGCAAACAGCTAATGCACATTGGCAACAGCC | |
| | CCTGATGCCTATGCCTTATTCATCCCTCAGAAAAGGATTCAAGTAG | |
| | AGGCTTGATTTGGAGGTTAAAGTTTTGCTATGCTGTATTTTACATTA | |
| | CTTATTGTTTTAGCTGTCCTCATGAATGTCTTTTCACTACCCATTTG | |
| | CTTATCCTGCATCTCTCAGCCTTGACTCCACTCAGTTCTCTTGCTTA | |
| | GAGATACCACCTTTCCCCTGAAGTGTTCCTTCCATGTTTTACGGCG | |
| | AGATGGTTTCTCCTCGCCTGGCCACTCAGCCTTAGTTGTCTCTGTTG | |
| | TCTTATAGAGGTCTACTTGAAGAAGGAAAAACAGGGGGCATGGTT | |
| | TGACTGTCCTGTGAGCCCTTCTTCCCTGCCTCCCCCACTCACAGTGA | |
| | CCCGGAATCCCTCGACATGGCAGTCTAGATCATTCTTGAAGACGAA | |
| | AGGGCCTCGTGATACGCCTATTTTTATAGGTTAATGTCATGATAAT | |
| | AATGGTTTCTTAGACGTCAGGTGGCACTTTTCGGGGAAATGTGCGC | |
| | GGAACCCCTATTTGTTTATTTTTCTAAATACATTCAAATATGTATCC | |
| | GCTCATGAGACAATAACCCTGATAAATGCTTCAATAATATTGAAA | |
| | AAGGAAGAGTATGAGTATTCAACATTTCCGTGTCGCCCTTATTCCC | |
| | TTTTTTGCGGCATTTTGCCTTCCTGTTTTTGCTCACCCAGAAACGCT | |
| | GGTGAAAGTAAAAGATGCTGAAGATCAGTTGGGTGCACGAGTGGG | |
| | TTACATCGAACTGGATCTCAACAGCGGTAAGATCCTTGAGAGTTTT | |
| | CGCCCCGAAGAACGTTTTCCAATGATGAGCACTTTTAAAGTTCTGC | |
| | TATGTGGCGCGGTATTATCCCGTATTGACGCCGGGCAAGAGCAACT | |
| | CGGTCGCCGCATACACTATTCTCAGAATGACTTGGTTGAGTACTCA | |
| | CCAGTCACAGAAAAGCATCTTACGGATGGCATGACAGTAAGAGAA | |
| | TTATGCAGTGCTGCCATAACCATGAGTGATAACACTGCGGCCAACT | |
| | TACTTCTGACAACGATCGGAGGACCGAAGGAGCTAACCGCTTTTTT | |
| | GCACAACATGGGGGATCATGTAACTCGCCTTGATCGTTGGGAACC | |
| | GGAGCTGAATGAAGCCATACCAAACGACGAGCGTGACACCACGAT | |
| | GCCTGTAGCAATGGCAACAACGTTGCGCAAACTATTAACTGGCGA | |
| | ACTACTTACTCTAGCTTCCCGGCAACAATTAATAGACTGGATGGAG | |
| | GCGGATAAAGTTGCAGGACCACTTCTGCGCTCGGCCCTTCCGGCTG | |
| | GCTGGTTTATTGCTGATAAATCTGGAGCCGGTGAGCGTGGGTCTCG | |
| | CGGTATCATTGCAGCACTGGGGCCAGATGGTAAGCCCTCCCGTATC | |
| | GTAGTTATCTACACGACGGGGAGTCAGGCAACTATGGATGAACGA | |
| | AATAGACAGATCGCTGAGATAGGTGCCTCACTGATTAAGCATTGG | |
| | TAACTGTCAGACCAAGTTTACTCATATATACTTTAGATTGATTTAA | |
| | AACTTCATTTTTAATTTAAAAGGATCTAGGTGAAGATCCTTTTTGAT | |
| | AATCTCATGACCAAAATCCCTTAACGTGAGTTTTCGTTCCACTGAG | |
| | CGTCAGACCCCGTAGAAAAGATCAAAGGATCTTCTTGAGATCCTTT | |
| | TTTTCTGCGCGTAATCTGCTGCTTGCAAACAAAAAAACCACCGCTA | |
| | CCAGCGGTGGTTTGTTTGCCGGATCAAGAGCTACCAACTCTTTTTC | |
| | CGAAGGTAACTGGCTTCAGCAGAGCGCAGATACCAAATACTGTTC | |
| | TTCTAGTGTAGCCGTAGTTAGGCCACCACTTCAAGAACTCTGTAGC | |
| | ACCGCCTACATACCTCGCTCTGCTAATCCTGTTACCAGTGGCTGCT | |
| | GCCAGTGGCGATAAGTCGTGTCTTACCGGGTTGGACTCAAGACGA | |
| | TAGTTACCGGATAAGGCGCAGCGGTCGGGCTGAACGGGGGGTTCG | |
| | TGCACACAGCCCAGCTTGGAGCGAACGACCTACACCGAACTGAGA | |
| | TACCTACAGCGTGAGCTATGAGAAAGCGCCACGCTTCCCGAAGGG | |
| | AGAAAGGCGGACAGGTATCCGGTAAGCGGCAGGGTCGGAACAGG | |
| | AGAGCGCACGAGGGAGCTTCCAGGGGGAAACGCCTGGTATCTTTA | |
| | TAGTCCTGTCGGGTTTCGCCACCTCTGACTTGAGCGTCGATTTTTGT | |
| | GATGCTCGTCAGGGGGGCGGAGCCTATGGAAAAACGCCAGCAACG | |
| | GATGCGCCGCGTGCGGCTGCTGGAGATGGCGGACGCGATGGATAT | |
| | GTTCTGCCAAGGGTTGGTTTGCGCATTCACAGTTCTCCGCAAGAAT | |
| | TGATTGGCTCCAATTCTTGGAGTGGTGAATCCGTTAGCGAGGTGCC | |
| | GCCGGCTTCCATTCAGGTCGAGGTGGCCCGGCTCCATGCACCGCGA | |
| | CGCAACGCGGGGAGGCAGACAAGGTATAGGGCGGCGCCTACAATC | |
| | CATGCCAACCCGTTCCATGTGCTCGCCGAGGCGGCATAAATCCCCG | |
| | TGACGATCAGCGGTCCAATGATCGAAGTTAGGCTGGTAAGAGCCG | |
| | CGAGCGATCCTTGAAGCTGTCCCTGATGGTCGTCATCTACCTGCCT | |
| | GGACAGCATGGCCTGCAACGCGGGCATCCCGATGCCGCCGGAAGC | |
| | GAGAAGAATCATAATGGGGAAGGCCATCCAGCCTCGCGTCGGGGA | |
| | GCTTTTTGCAAAAGCCTAGGCCTCCAAAAAAGCCTCCTCACTACTT | |
| | CTGGAATAGCTCAGAGGCCGAGGCGGCCTCGGCCTCTGCATAAAT | |
| | AAAAAAAATTAGTCAGCCATG | |
| 64 | AGCTTGGCCCATTGCATACGTTGTATCCATATCATAATATGTACAT | C-term PH |
| | TTATATTGGCTCATGTCCAACATTACCGCCATGTTGACATTGATTAT | domain |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGTCCAATTTAC | |
| | TGACCGTACACCAAAATTTGCCTGCATTACCGGTCGATGCAACGAG | |
| | TGATGAGGTTCGCAAGAACCTGATGGACATGTTCAGGGATCGCCA | |
| | GGCGTTTTCTGAGCATACCTGGAAAATGCTTCTGTCCGTTTGCCGG | |
| | TCGTGGGCGGCATGGTGCAAGTTGAATAACCGGAAATGGTTTCCC | |
| | GCAGAACCTGAAGATGTTCGCGATTATCTTCTATATCTTCAGGCGC | |
| | GCGGTCTGGCAGTAAAAACTATCCAGCAACATTTGGGCCAGCTAA | |
| | ACATGCTTCATCGTCGGTCCGGGCTGCCACGACCAAGTGACAGCA | |
| | ATGCTGTTTCACTGGTTATGCGGCGGATCCGAAAAGAAAACGTTG | |
| | ATGCCGGTGAACGTGCAAAACAGGCTCTAGCGTTCGAACGCACTG | |
| | ATTTCGACCAGGTTCGTTCACTCATGGAAAATAGCGATCGCTGCCA | |
| | GGATATACGTAATCTGGCATTTCTGGGGATTGCTTATAACACCCTG | |
| | TTACGTATAGCCGAAATTGCCAGGATCAGGGTTAAAGATATCTCAC | |
| | GTACTGACGGTGGGAGAATGTTAATCCATATTGGCAGAACGAAAA | |
| | CGCTGGTTAGCACCGCAGGTGTAGAGAAGGCACTTAGCCTGGGGG | |
| | TAACTAAACTGGTCGAGCGATGGATTTCCGTCTCTGGTGTAGCTGA | |
| | TGATCCGAATAACTACCTGTTTTGCCGGGTCAGAAAAAATGGTGTT | |
| | GCCGCGCCATCTGCCACCAGCCAGCTATCAACTCGCGCCCTGGAA | |
| | GGGATTTTTGAAGCAACTCATCGATTGATTTACGGCGCTAAGGATG | |
| | ACTCTGGTCAGAGATACCTGGCCTGGTCTGGACACAGTGCCCGTGT | |
| | CGGAGCCGCGCGAGATATGGCCCGCGCTGGAGTTTCAATACCGGA | |
| | GATCATGCAAGCTGGTGGCTGGACCAATGTAAATATTGTCATGAA | |
| | CTATATCCGTAACCTGGATAGTGAAACAGGGGCAATGGTGCGCCT | |
| | GCTGGAAGATGGCGATTCCCAGAACTACCCCATCGTGCAGATGGA | |
| | CAGCGGGAGGGATTTCCTCACGCTGCACGGTTTGCAGGACGACGA | |
| | GGACCTCCAAGCTTTGCTCAAAGGCAGTCAACTTCTCAAGGTCAAG | |
| | AGCAGCTCCTGGAGGCGCGAACGGTTTTACAAGTTGCAAGAAGAT | |
| | TGCAAAACAATTTGGCAAGAGAGCCGGAAAGTCATGAGAACTCCC | |
| | GAGAGCCAACTGTTCAGCATCGAGGACATCCAAGAAGTCAGGATG | |
| | GGCCATAGGACCGAGGGCTTGGAAAAATTCGCCAGGGACGTGCCC | |
| | GAAGACCGATGCTTTAGCATCGTGTTCAAAGATCAGAGGAACACG | |
| | TTGGACTTGATCGCCCCCAGTCCGGCTGATGCCCAGCATTGGGTTC | |
| | TGGGGCTCCATAAGATCATCCATCATAGCGGCAGCATGGACCAGA | |
| | GGCAGAAACTCCAACATTGGATTCATAGTTGTCTTAGGAAAGCCG | |
| | ACAAGAACAAGGACAACAAGATGAGCTTCAAGGAGTTGCAGAATT | |
| | TTCTTAAAGAGCTGAATATCCAGTAAGGATCCAAGCTTATCGATAC | |
| | CGTCGACCTCGAGGGCCCAGATCTAATTCACCCCACCAGTGCAGG | |
| | CTGCCTATCAGAAAGTGGTGGCTGGTGTGGCTAATGCCCTGGCCCA | |
| | CAAGTATCACTAAGCTCGCTTTCTTGCTGTCCAATTTCTATTAAAG | |
| | GTTCCTTTGTTCCCTAAGTCCAACTACTAAACTGGGGGATATTATG | |
| | AAGGGCCTTGAGCATCTGGATTCTGCCTAATAAAAAACATTTATTT | |
| | TCATTGCAATGATGTATTTAAATTATTTCTGAATATTTTACTAAAAA | |
| | GGGAATGTGGGAGGTCAGTGCATTTAAAACATAAAGAAATGAAGA | |
| | GCTAGTTCAAACCTTGGGAAAATACACTATATCTTAAACTCCATGA | |
| | AAGAAGGTGAGGCTGCAAACAGCTAATGCACATTGGCAACAGCCC | |
| | CTGATGCCTATGCCTTATTCATCCCTCAGAAAAGGATTCAAGTAGA | |
| | GGCTTGATTTGGAGGTTAAAGTTTTGCTATGCTGTATTTTACATTAC | |
| | TTATTGTTTTAGCTGTCCTCATGAATGTCTTTTCACTACCCATTTGC | |
| | TTATCCTGCATCTCTCAGCCTTGACTCCACTCAGTTCTCTTGCTTAG | |
| | AGATACCACCTTTCCCCTGAAGTGTTCCTTCCATGTTTTACGGCGA | |
| | GATGGTTTCTCCTCGCCTGGCCACTCAGCCTTAGTTGTCTCTGTTGT | |
| | CTTATAGAGGTCTACTTGAAGAAGGAAAAACAGGGGGCATGGTTT | |
| | GACTGTCCTGTGAGCCCTTCTTCCCTGCCTCCCCCACTCACAGTGA | |
| | CCCGGAATCCCTCGACATGGCAGTCTAGATCATTCTTGAAGACGAA | |
| | AGGGCCTCGTGATACGCCTATTTTTATAGGTTAATGTCATGATAAT | |
| | AATGGTTTCTTAGACGTCAGGTGGCACTTTTCGGGGAAATGTGCGC | |
| | GGAACCCCTATTTGTTTATTTTTCTAAATACATTCAAATATGTATCC | |
| | GCTCATGAGACAATAACCCTGATAAATGCTTCAATAATATTGAAA | |
| | AAGGAAGAGTATGAGTATTCAACATTTCCGTGTCGCCCTTATTCCC | |
| | TTTTTTGCGGCATTTTGCCTTCCTGTTTTTGCTCACCCAGAAACGCT | |
| | GGTGAAAGTAAAAGATGCTGAAGATCAGTTGGGTGCACGAGTGGG | |
| | TTACATCGAACTGGATCTCAACAGCGGTAAGATCCTTGAGAGTTTT | |
| | CGCCCCGAAGAACGTTTTCCAATGATGAGCACTTTTAAAGTTCTGC | |
| | TATGTGGCGCGGTATTATCCCGTATTGACGCCGGGCAAGAGCAACT | |
| | CGGTCGCCGCATACACTATTCTCAGAATGACTTGGTTGAGTACTCA | |
| | CCAGTCACAGAAAAGCATCTTACGGATGGCATGACAGTAAGAGAA | |
| | TTATGCAGTGCTGCCATAACCATGAGTGATAACACTGCGGCCAACT | |
| | TACTTCTGACAACGATCGGAGGACCGAAGGAGCTAACCGCTTTTTT | |
| | GCACAACATGGGGGATCATGTAACTCGCCTTGATCGTTGGGAACC | |
| | GGAGCTGAATGAAGCCATACCAAACGACGAGCGTGACACCACGAT | |
| | GCCTGTAGCAATGGCAACAACGTTGCGCAAACTATTAACTGGCGA | |
| | ACTACTTACTCTAGCTTCCCGGCAACAATTAATAGACTGGATGGAG | |
| | GCGGATAAAGTTGCAGGACCACTTCTGCGCTCGGCCCTTCCGGCTG | |
| | GCTGGTTTATTGCTGATAAATCTGGAGCCGGTGAGCGTGGGTCTCG | |
| | CGGTATCATTGCAGCACTGGGGCCAGATGGTAAGCCCTCCCGTATC | |
| | GTAGTTATCTACACGACGGGGAGTCAGGCAACTATGGATGAACGA | |
| | AATAGACAGATCGCTGAGATAGGTGCCTCACTGATTAAGCATTGG | |
| | TAACTGTCAGACCAAGTTTACTCATATATACTTTAGATTGATTTAA | |
| | AACTTCATTTTTAATTTAAAAGGATCTAGGTGAAGATCCTTTTTGAT | |
| | AATCTCATGACCAAAATCCCTTAACGTGAGTTTTCGTTCCACTGAG | |
| | CGTCAGACCCCGTAGAAAAGATCAAAGGATCTTCTTGAGATCCTTT | |
| | TTTTCTGCGCGTAATCTGCTGCTTGCAAACAAAAAAACCACCGCTA | |
| | CCAGCGGTGGTTTGTTTGCCGGATCAAGAGCTACCAACTCTTTTTC | |
| | CGAAGGTAACTGGCTTCAGCAGAGCGCAGATACCAAATACTGTTC | |
| | TTCTAGTGTAGCCGTAGTTAGGCCACCACTTCAAGAACTCTGTAGC | |
| | ACCGCCTACATACCTCGCTCTGCTAATCCTGTTACCAGTGGCTGCT | |
| | GCCAGTGGCGATAAGTCGTGTCTTACCGGGTTGGACTCAAGACGA | |
| | TAGTTACCGGATAAGGCGCAGCGGTCGGGCTGAACGGGGGGTTCG | |
| | TGCACACAGCCCAGCTTGGAGCGAACGACCTACACCGAACTGAGA | |
| | TACCTACAGCGTGAGCTATGAGAAAGCGCCACGCTTCCCGAAGGG | |
| | AGAAAGGCGGACAGGTATCCGGTAAGCGGCAGGGTCGGAACAGG | |
| | AGAGCGCACGAGGGAGCTTCCAGGGGGAAACGCCTGGTATCTTTA | |
| | TAGTCCTGTCGGGTTTCGCCACCTCTGACTTGAGCGTCGATTTTTGT | |
| | GATGCTCGTCAGGGGGGCGGAGCCTATGGAAAAACGCCAGCAACG | |
| | GATGCGCCGCGTGCGGCTGCTGGAGATGGCGGACGCGATGGATAT | |
| | GTTCTGCCAAGGGTTGGTTTGCGCATTCACAGTTCTCCGCAAGAAT | |
| | TGATTGGCTCCAATTCTTGGAGTGGTGAATCCGTTAGCGAGGTGCC | |
| | GCCGGCTTCCATTCAGGTCGAGGTGGCCCGGCTCCATGCACCGCGA | |
| | CGCAACGCGGGGAGGCAGACAAGGTATAGGGCGGCGCCTACAATC | |
| | CATGCCAACCCGTTCCATGTGCTCGCCGAGGCGGCATAAATCCCCG | |
| | TGACGATCAGCGGTCCAATGATCGAAGTTAGGCTGGTAAGAGCCG | |
| | CGAGCGATCCTTGAAGCTGTCCCTGATGGTCGTCATCTACCTGCCT | |
| | GGACAGCATGGCCTGCAACGCGGGCATCCCGATGCCGCCGGAAGC | |
| | GAGAAGAATCATAATGGGGAAGGCCATCCAGCCTCGCGTCGGGGA | |
| | GCTTTTTGCAAAAGCCTAGGCCTCCAAAAAAGCCTCCTCACTACTT | |
| | CTGGAATAGCTCAGAGGCCGAGGCGGCCTCGGCCTCTGCATAAAT | |
| | AAAAAAAATTAGTCAGCCATG | |
| 65 | AGCTTGGCCCATTGCATACGTTGTATCCATATCATAATATGTACAT | Myr (n-term; |
| | TTATATTGGCTCATGTCCAACATTACCGCCATGTTGACATTGATTAT | lyn pal⁻) |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCATC | |
| | AAATCAAAGAGGAAGGATAATCTGAATGACGATGAATCCCAGAAC | |
| | TACCCCATCGTGCAGATGTCCAATTTACTGACCGTACACCAAAATT | |
| | TGCCTGCATTACCGGTCGATGCAACGAGTGATGAGGTTCGCAAGA | |
| | ACCTGATGGACATGTTCAGGGATCGCCAGGCGTTTTCTGAGCATAC | |
| | CTGGAAAATGCTTCTGTCCGTTTGCCGGTCGTGGGCGGCATGGTGC | |
| | AAGTTGAATAACCGGAAATGGTTTCCCGCAGAACCTGAAGATGTT | |
| | CGCGATTATCTTCTATATCTTCAGGCGCGCGGTCTGGCAGTAAAAA | |
| | CTATCCAGCAACATTTGGGCCAGCTAAACATGCTTCATCGTCGGTC | |
| | CGGGCTGCCACGACCAAGTGACAGCAATGCTGTTTCACTGGTTATG | |
| | CGGCGGATCCGAAAAGAAAACGTTGATGCCGGTGAACGTGCAAAA | |
| | CAGGCTCTAGCGTTCGAACGCACTGATTTCGACCAGGTTCGTTCAC | |
| | TCATGGAAAATAGCGATCGCTGCCAGGATATACGTAATCTGGCATT | |
| | TCTGGGGATTGCTTATAACACCCTGTTACGTATAGCCGAAATTGCC | |
| | AGGATCAGGGTTAAAGATATCTCACGTACTGACGGTGGGAGAATG | |
| | TTAATCCATATTGGCAGAACGAAAACGCTGGTTAGCACCGCAGGT | |
| | GTAGAGAAGGCACTTAGCCTGGGGGTAACTAAACTGGTCGAGCGA | |
| | TGGATTTCCGTCTCTGGTGTAGCTGATGATCCGAATAACTACCTGT | |
| | TTTGCCGGGTCAGAAAAAATGGTGTTGCCGCGCCATCTGCCACCAG | |
| | CCAGCTATCAACTCGCGCCCTGGAAGGGATTTTTGAAGCAACTCAT | |
| | CGATTGATTTACGGCGCTAAGGATGACTCTGGTCAGAGATACCTGG | |
| | CCTGGTCTGGACACAGTGCCCGTGTCGGAGCCGCGCGAGATATGG | |
| | CCCGCGCTGGAGTTTCAATACCGGAGATCATGCAAGCTGGTGGCT | |
| | GGACCAATGTAAATATTGTCATGAACTATATCCGTAACCTGGATAG | |
| | TGAAACAGGGGCAATGGTGCGCCTGCTGGAAGATGGCGATTAAGG | |
| | ATCCAAGCTTATCGATACCGTCGACCTCGAGGGCCCAGATCTAATT | |
| | CACCCCACCAGTGCAGGCTGCCTATCAGAAAGTGGTGGCTGGTGT | |
| | GGCTAATGCCCTGGCCCACAAGTATCACTAAGCTCGCTTTCTTGCT | |
| | GTCCAATTTCTATTAAAGGTTCCTTTGTTCCCTAAGTCCAACTACTA | |
| | AACTGGGGGATATTATGAAGGGCCTTGAGCATCTGGATTCTGCCTA | |
| | ATAAAAAACATTTATTTTCATTGCAATGATGTATTTAAATTATTTCT | |
| | GAATATTTTACTAAAAAGGGAATGTGGGAGGTCAGTGCATTTAAA | |
| | ACATAAAGAAATGAAGAGCTAGTTCAAACCTTGGGAAAATACACT | |
| | ATATCTTAAACTCCATGAAAGAAGGTGAGGCTGCAAACAGCTAAT | |
| | GCACATTGGCAACAGCCCCTGATGCCTATGCCTTATTCATCCCTCA | |
| | GAAAAGGATTCAAGTAGAGGCTTGATTTGGAGGTTAAAGTTTTGCT | |
| | ATGCTGTATTTTACATTACTTATTGTTTTAGCTGTCCTCATGAATGT | |
| | CTTTTCACTACCCATTTGCTTATCCTGCATCTCTCAGCCTTGACTCC | |
| | ACTCAGTTCTCTTGCTTAGAGATACCACCTTTCCCCTGAAGTGTTCC | |
| | TTCCATGTTTTACGGCGAGATGGTTTCTCCTCGCCTGGCCACTCAG | |
| | CCTTAGTTGTCTCTGTTGTCTTATAGAGGTCTACTTGAAGAAGGAA | |
| | AAACAGGGGGCATGGTTTGACTGTCCTGTGAGCCCTTCTTCCCTGC | |
| | CTCCCCCACTCACAGTGACCCGGAATCCCTCGACATGGCAGTCTAG | |
| | ATCATTCTTGAAGACGAAAGGGCCTCGTGATACGCCTATTTTTATA | |
| | GGTTAATGTCATGATAATAATGGTTTCTTAGACGTCAGGTGGCACT | |
| | TTTCGGGGAAATGTGCGCGGAACCCCTATTTGTTTATTTTTCTAAAT | |
| | ACATTCAAATATGTATCCGCTCATGAGACAATAACCCTGATAAATG | |
| | CTTCAATAATATTGAAAAAGGAAGAGTATGAGTATTCAACATTTCC | |
| | GTGTCGCCCTTATTCCCTTTTTTGCGGCATTTTGCCTTCCTGTTTTTG | |
| | CTCACCCAGAAACGCTGGTGAAAGTAAAAGATGCTGAAGATCAGT | |
| | TGGGTGCACGAGTGGGTTACATCGAACTGGATCTCAACAGCGGTA | |
| | AGATCCTTGAGAGTTTTCGCCCCGAAGAACGTTTTCCAATGATGAG | |
| | CACTTTTAAAGTTCTGCTATGTGGCGCGGTATTATCCCGTATTGAC | |
| | GCCGGGCAAGAGCAACTCGGTCGCCGCATACACTATTCTCAGAAT | |
| | GACTTGGTTGAGTACTCACCAGTCACAGAAAAGCATCTTACGGAT | |
| | GGCATGACAGTAAGAGAATTATGCAGTGCTGCCATAACCATGAGT | |
| | GATAACACTGCGGCCAACTTACTTCTGACAACGATCGGAGGACCG | |
| | AAGGAGCTAACCGCTTTTTTGCACAACATGGGGGATCATGTAACTC | |
| | GCCTTGATCGTTGGGAACCGGAGCTGAATGAAGCCATACCAAACG | |
| | ACGAGCGTGACACCACGATGCCTGTAGCAATGGCAACAACGTTGC | |
| | GCAAACTATTAACTGGCGAACTACTTACTCTAGCTTCCCGGCAACA | |
| | ATTAATAGACTGGATGGAGGCGGATAAAGTTGCAGGACCACTTCT | |
| | GCGCTCGGCCCTTCCGGCTGGCTGGTTTATTGCTGATAAATCTGGA | |
| | GCCGGTGAGCGTGGGTCTCGCGGTATCATTGCAGCACTGGGGCCA | |
| | GATGGTAAGCCCTCCCGTATCGTAGTTATCTACACGACGGGGAGTC | |
| | AGGCAACTATGGATGAACGAAATAGACAGATCGCTGAGATAGGTG | |
| | CCTCACTGATTAAGCATTGGTAACTGTCAGACCAAGTTTACTCATA | |
| | TATACTTTAGATTGATTTAAAACTTCATTTTTAATTTAAAAGGATCT | |
| | AGGTGAAGATCCTTTTTGATAATCTCATGACCAAAATCCCTTAACG | |
| | TGAGTTTTCGTTCCACTGAGCGTCAGACCCCGTAGAAAAGATCAAA | |
| | GGATCTTCTTGAGATCCTTTTTTTCTGCGCGTAATCTGCTGCTTGCA | |
| | AACAAAAAAACCACCGCTACCAGCGGTGGTTTGTTTGCCGGATCA | |
| | AGAGCTACCAACTCTTTTTCCGAAGGTAACTGGCTTCAGCAGAGCG | |
| | CAGATACCAAATACTGTTCTTCTAGTGTAGCCGTAGTTAGGCCACC | |
| | ACTTCAAGAACTCTGTAGCACCGCCTACATACCTCGCTCTGCTAAT | |
| | CCTGTTACCAGTGGCTGCTGCCAGTGGCGATAAGTCGTGTCTTACC | |
| | GGGTTGGACTCAAGACGATAGTTACCGGATAAGGCGCAGCGGTCG | |
| | GGCTGAACGGGGGGTTCGTGCACACAGCCCAGCTTGGAGCGAACG | |
| | ACCTACACCGAACTGAGATACCTACAGCGTGAGCTATGAGAAAGC | |
| | GCCACGCTTCCCGAAGGGAGAAAGGCGGACAGGTATCCGGTAAGC | |
| | GGCAGGGTCGGAACAGGAGAGCGCACGAGGGAGCTTCCAGGGGG | |
| | AAACGCCTGGTATCTTTATAGTCCTGTCGGGTTTCGCCACCTCTGA | |
| | CTTGAGCGTCGATTTTTGTGATGCTCGTCAGGGGGGCGGAGCCTAT | |
| | GGAAAAACGCCAGCAACGGATGCGCCGCGTGCGGCTGCTGGAGAT | |
| | GGCGGACGCGATGGATATGTTCTGCCAAGGGTTGGTTTGCGCATTC | |
| | ACAGTTCTCCGCAAGAATTGATTGGCTCCAATTCTTGGAGTGGTGA | |
| | ATCCGTTAGCGAGGTGCCGCCGGCTTCCATTCAGGTCGAGGTGGCC | |
| | CGGCTCCATGCACCGCGACGCAACGCGGGGAGGCAGACAAGGTAT | |
| | AGGGCGGCGCCTACAATCCATGCCAACCCGTTCCATGTGCTCGCCG | |
| | AGGCGGCATAAATCCCCGTGACGATCAGCGGTCCAATGATCGAAG | |
| | TTAGGCTGGTAAGAGCCGCGAGCGATCCTTGAAGCTGTCCCTGATG | |
| | GTCGTCATCTACCTGCCTGGACAGCATGGCCTGCAACGCGGGCATC | |
| | CCGATGCCGCCGGAAGCGAGAAGAATCATAATGGGGAAGGCCATC | |
| | CAGCCTCGCGTCGGGGAGCTTTTTGCAAAAGCCTAGGCCTCCAAA | |
| | AAAGCCTCCTCACTACTTCTGGAATAGCTCAGAGGCCGAGGCGGC | |
| | CTCGGCCTCTGCATAAATAAAAAAAATTAGTCAGCCATG | |
| 66 | AGCTTGGCCCATTGCATACGTTGTATCCATATCATAATATGTACAT | Single Pal, |
| | TTATATTGGCTCATGTCCAACATTACCGCCATGTTGACATTGATTAT | from GNA12 |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGTCCGGGGTG | |
| | GTGCGGACCCTCAGCCGCTGCCTGCTGCCGGCCGAGGCCGGCTCCC | |
| | AGAACTACCCCATCGTGCAGATGTCCAATTTACTGACCGTACACCA | |
| | AAATTTGCCTGCATTACCGGTCGATGCAACGAGTGATGAGGTTCGC | |
| | AAGAACCTGATGGACATGTTCAGGGATCGCCAGGCGTTTTCTGAG | |
| | CATACCTGGAAAATGCTTCTGTCCGTTTGCCGGTCGTGGGCGGCAT | |
| | GGTGCAAGTTGAATAACCGGAAATGGTTTCCCGCAGAACCTGAAG | |
| | ATGTTCGCGATTATCTTCTATATCTTCAGGCGCGCGGTCTGGCAGT | |
| | AAAAACTATCCAGCAACATTTGGGCCAGCTAAACATGCTTCATCGT | |
| | CGGTCCGGGCTGCCACGACCAAGTGACAGCAATGCTGTTTCACTG | |
| | GTTATGCGGCGGATCCGAAAAGAAAACGTTGATGCCGGTGAACGT | |
| | GCAAAACAGGCTCTAGCGTTCGAACGCACTGATTTCGACCAGGTTC | |
| | GTTCACTCATGGAAAATAGCGATCGCTGCCAGGATATACGTAATCT | |
| | GGCATTTCTGGGGATTGCTTATAACACCCTGTTACGTATAGCCGAA | |
| | ATTGCCAGGATCAGGGTTAAAGATATCTCACGTACTGACGGTGGG | |
| | AGAATGTTAATCCATATTGGCAGAACGAAAACGCTGGTTAGCACC | |
| | GCAGGTGTAGAGAAGGCACTTAGCCTGGGGGTAACTAAACTGGTC | |
| | GAGCGATGGATTTCCGTCTCTGGTGTAGCTGATGATCCGAATAACT | |
| | ACCTGTTTTGCCGGGTCAGAAAAAATGGTGTTGCCGCGCCATCTGC | |
| | CACCAGCCAGCTATCAACTCGCGCCCTGGAAGGGATTTTTGAAGC | |
| | AACTCATCGATTGATTTACGGCGCTAAGGATGACTCTGGTCAGAGA | |
| | TACCTGGCCTGGTCTGGACACAGTGCCCGTGTCGGAGCCGCGCGA | |
| | GATATGGCCCGCGCTGGAGTTTCAATACCGGAGATCATGCAAGCT | |
| | GGTGGCTGGACCAATGTAAATATTGTCATGAACTATATCCGTAACC | |
| | TGGATAGTGAAACAGGGGCAATGGTGCGCCTGCTGGAAGATGGCG | |
| | ATTAAGGATCCAAGCTTATCGATACCGTCGACCTCGAGGGCCCAG | |
| | ATCTAATTCACCCCACCAGTGCAGGCTGCCTATCAGAAAGTGGTGG | |
| | CTGGTGTGGCTAATGCCCTGGCCCACAAGTATCACTAAGCTCGCTT | |
| | TCTTGCTGTCCAATTTCTATTAAAGGTTCCTTTGTTCCCTAAGTCCA | |
| | ACTACTAAACTGGGGGATATTATGAAGGGCCTTGAGCATCTGGATT | |
| | CTGCCTAATAAAAAACATTTATTTTCATTGCAATGATGTATTTAAA | |
| | TTATTTCTGAATATTTTACTAAAAAGGGAATGTGGGAGGTCAGTGC | |
| | ATTTAAAACATAAAGAAATGAAGAGCTAGTTCAAACCTTGGGAAA | |
| | ATACACTATATCTTAAACTCCATGAAAGAAGGTGAGGCTGCAAAC | |
| | AGCTAATGCACATTGGCAACAGCCCCTGATGCCTATGCCTTATTCA | |
| | TCCCTCAGAAAAGGATTCAAGTAGAGGCTTGATTTGGAGGTTAAA | |
| | GTTTTGCTATGCTGTATTTTACATTACTTATTGTTTTAGCTGTCCTCA | |
| | TGAATGTCTTTTCACTACCCATTTGCTTATCCTGCATCTCTCAGCCT | |
| | TGACTCCACTCAGTTCTCTTGCTTAGAGATACCACCTTTCCCCTGAA | |
| | GTGTTCCTTCCATGTTTTACGGCGAGATGGTTTCTCCTCGCCTGGCC | |
| | ACTCAGCCTTAGTTGTCTCTGTTGTCTTATAGAGGTCTACTTGAAG | |
| | AAGGAAAAACAGGGGGCATGGTTTGACTGTCCTGTGAGCCCTTCTT | |
| | CCCTGCCTCCCCCACTCACAGTGACCCGGAATCCCTCGACATGGCA | |
| | GTCTAGATCATTCTTGAAGACGAAAGGGCCTCGTGATACGCCTATT | |
| | TTTATAGGTTAATGTCATGATAATAATGGTTTCTTAGACGTCAGGT | |
| | GGCACTTTTCGGGGAAATGTGCGCGGAACCCCTATTTGTTTATTTT | |
| | TCTAAATACATTCAAATATGTATCCGCTCATGAGACAATAACCCTG | |
| | ATAAATGCTTCAATAATATTGAAAAAGGAAGAGTATGAGTATTCA | |
| | ACATTTCCGTGTCGCCCTTATTCCCTTTTTTGCGGCATTTTGCCTTC | |
| | CTGTTTTTGCTCACCCAGAAACGCTGGTGAAAGTAAAAGATGCTGA | |
| | AGATCAGTTGGGTGCACGAGTGGGTTACATCGAACTGGATCTCAA | |
| | CAGCGGTAAGATCCTTGAGAGTTTTCGCCCCGAAGAACGTTTTCCA | |
| | ATGATGAGCACTTTTAAAGTTCTGCTATGTGGCGCGGTATTATCCC | |
| | GTATTGACGCCGGGCAAGAGCAACTCGGTCGCCGCATACACTATT | |
| | CTCAGAATGACTTGGTTGAGTACTCACCAGTCACAGAAAAGCATCT | |
| | TACGGATGGCATGACAGTAAGAGAATTATGCAGTGCTGCCATAAC | |
| | CATGAGTGATAACACTGCGGCCAACTTACTTCTGACAACGATCGG | |
| | AGGACCGAAGGAGCTAACCGCTTTTTTGCACAACATGGGGGATCA | |
| | TGTAACTCGCCTTGATCGTTGGGAACCGGAGCTGAATGAAGCCAT | |
| | ACCAAACGACGAGCGTGACACCACGATGCCTGTAGCAATGGCAAC | |
| | AACGTTGCGCAAACTATTAACTGGCGAACTACTTACTCTAGCTTCC | |
| | CGGCAACAATTAATAGACTGGATGGAGGCGGATAAAGTTGCAGGA | |
| | CCACTTCTGCGCTCGGCCCTTCCGGCTGGCTGGTTTATTGCTGATA | |
| | AATCTGGAGCCGGTGAGCGTGGGTCTCGCGGTATCATTGCAGCACT | |
| | GGGGCCAGATGGTAAGCCCTCCCGTATCGTAGTTATCTACACGACG | |
| | GGGAGTCAGGCAACTATGGATGAACGAAATAGACAGATCGCTGAG | |
| | ATAGGTGCCTCACTGATTAAGCATTGGTAACTGTCAGACCAAGTTT | |
| | ACTCATATATACTTTAGATTGATTTAAAACTTCATTTTTAATTTAAA | |
| | AGGATCTAGGTGAAGATCCTTTTTGATAATCTCATGACCAAAATCC | |
| | CTTAACGTGAGTTTTCGTTCCACTGAGCGTCAGACCCCGTAGAAAA | |
| | GATCAAAGGATCTTCTTGAGATCCTTTTTTTCTGCGCGTAATCTGCT | |
| | GCTTGCAAACAAAAAAACCACCGCTACCAGCGGTGGTTTGTTTGCC | |
| | GGATCAAGAGCTACCAACTCTTTTTCCGAAGGTAACTGGCTTCAGC | |
| | AGAGCGCAGATACCAAATACTGTTCTTCTAGTGTAGCCGTAGTTAG | |
| | GCCACCACTTCAAGAACTCTGTAGCACCGCCTACATACCTCGCTCT | |
| | GCTAATCCTGTTACCAGTGGCTGCTGCCAGTGGCGATAAGTCGTGT | |
| | CTTACCGGGTTGGACTCAAGACGATAGTTACCGGATAAGGCGCAG | |
| | CGGTCGGGCTGAACGGGGGGTTCGTGCACACAGCCCAGCTTGGAG | |
| | CGAACGACCTACACCGAACTGAGATACCTACAGCGTGAGCTATGA | |
| | GAAAGCGCCACGCTTCCCGAAGGGAGAAAGGCGGACAGGTATCCG | |
| | GTAAGCGGCAGGGTCGGAACAGGAGAGCGCACGAGGGAGCTTCC | |
| | AGGGGGAAACGCCTGGTATCTTTATAGTCCTGTCGGGTTTCGCCAC | |
| | CTCTGACTTGAGCGTCGATTTTTGTGATGCTCGTCAGGGGGGCGGA | |
| | GCCTATGGAAAAACGCCAGCAACGGATGCGCCGCGTGCGGCTGCT | |
| | GGAGATGGCGGACGCGATGGATATGTTCTGCCAAGGGTTGGTTTG | |
| | CGCATTCACAGTTCTCCGCAAGAATTGATTGGCTCCAATTCTTGGA | |
| | GTGGTGAATCCGTTAGCGAGGTGCCGCCGGCTTCCATTCAGGTCGA | |
| | GGTGGCCCGGCTCCATGCACCGCGACGCAACGCGGGGAGGCAGAC | |
| | AAGGTATAGGGCGGCGCCTACAATCCATGCCAACCCGTTCCATGT | |
| | GCTCGCCGAGGCGGCATAAATCCCCGTGACGATCAGCGGTCCAAT | |
| | GATCGAAGTTAGGCTGGTAAGAGCCGCGAGCGATCCTTGAAGCTG | |
| | TCCCTGATGGTCGTCATCTACCTGCCTGGACAGCATGGCCTGCAAC | |
| | GCGGGCATCCCGATGCCGCCGGAAGCGAGAAGAATCATAATGGGG | |
| | AAGGCCATCCAGCCTCGCGTCGGGGAGCTTTTTGCAAAAGCCTAG | |
| | GCCTCCAAAAAAGCCTCCTCACTACTTCTGGAATAGCTCAGAGGCC | |
| | GAGGCGGCCTCGGCCTCTGCATAAATAAAAAAAATTAGTCAGCCA | |
| | TG | |
| 67 | AGCTTGGCCCATTGCATACGTTGTATCCATATCATAATATGTACAT | Double Pal, |
| | TTATATTGGCTCATGTCCAACATTACCGCCATGTTGACATTGATTAT | from GNA13 |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGCGGACTTC | |
| | CTGCCGTCGCGGTCCGTGCTGTCCGTGTGCTTCCCCGGCTGCCTGC | |
| | TGACGAGTTCCCAGAACTACCCCATCGTGCAGATGTCCAATTTACT | |
| | GACCGTACACCAAAATTTGCCTGCATTACCGGTCGATGCAACGAGT | |
| | GATGAGGTTCGCAAGAACCTGATGGACATGTTCAGGGATCGCCAG | |
| | GCGTTTTCTGAGCATACCTGGAAAATGCTTCTGTCCGTTTGCCGGT | |
| | CGTGGGCGGCATGGTGCAAGTTGAATAACCGGAAATGGTTTCCCG | |
| | CAGAACCTGAAGATGTTCGCGATTATCTTCTATATCTTCAGGCGCG | |
| | CGGTCTGGCAGTAAAAACTATCCAGCAACATTTGGGCCAGCTAAA | |
| | CATGCTTCATCGTCGGTCCGGGCTGCCACGACCAAGTGACAGCAAT | |
| | GCTGTTTCACTGGTTATGCGGCGGATCCGAAAAGAAAACGTTGAT | |
| | GCCGGTGAACGTGCAAAACAGGCTCTAGCGTTCGAACGCACTGAT | |
| | TTCGACCAGGTTCGTTCACTCATGGAAAATAGCGATCGCTGCCAGG | |
| | ATATACGTAATCTGGCATTTCTGGGGATTGCTTATAACACCCTGTT | |
| | ACGTATAGCCGAAATTGCCAGGATCAGGGTTAAAGATATCTCACG | |
| | TACTGACGGTGGGAGAATGTTAATCCATATTGGCAGAACGAAAAC | |
| | GCTGGTTAGCACCGCAGGTGTAGAGAAGGCACTTAGCCTGGGGGT | |
| | AACTAAACTGGTCGAGCGATGGATTTCCGTCTCTGGTGTAGCTGAT | |
| | GATCCGAATAACTACCTGTTTTGCCGGGTCAGAAAAAATGGTGTTG | |
| | CCGCGCCATCTGCCACCAGCCAGCTATCAACTCGCGCCCTGGAAG | |
| | GGATTTTTGAAGCAACTCATCGATTGATTTACGGCGCTAAGGATGA | |
| | CTCTGGTCAGAGATACCTGGCCTGGTCTGGACACAGTGCCCGTGTC | |
| | GGAGCCGCGCGAGATATGGCCCGCGCTGGAGTTTCAATACCGGAG | |
| | ATCATGCAAGCTGGTGGCTGGACCAATGTAAATATTGTCATGAACT | |
| | ATATCCGTAACCTGGATAGTGAAACAGGGGCAATGGTGCGCCTGC | |
| | TGGAAGATGGCGATTAAGGATCCAAGCTTATCGATACCGTCGACC | |
| | TCGAGGGCCCAGATCTAATTCACCCCACCAGTGCAGGCTGCCTATC | |
| | AGAAAGTGGTGGCTGGTGTGGCTAATGCCCTGGCCCACAAGTATC | |
| | ACTAAGCTCGCTTTCTTGCTGTCCAATTTCTATTAAAGGTTCCTTTG | |
| | TTCCCTAAGTCCAACTACTAAACTGGGGGATATTATGAAGGGCCTT | |
| | GAGCATCTGGATTCTGCCTAATAAAAAACATTTATTTTCATTGCAA | |
| | TGATGTATTTAAATTATTTCTGAATATTTTACTAAAAAGGGAATGT | |
| | GGGAGGTCAGTGCATTTAAAACATAAAGAAATGAAGAGCTAGTTC | |
| | AAACCTTGGGAAAATACACTATATCTTAAACTCCATGAAAGAAGG | |
| | TGAGGCTGCAAACAGCTAATGCACATTGGCAACAGCCCCTGATGC | |
| | CTATGCCTTATTCATCCCTCAGAAAAGGATTCAAGTAGAGGCTTGA | |
| | TTTGGAGGTTAAAGTTTTGCTATGCTGTATTTTACATTACTTATTGT | |
| | TTTAGCTGTCCTCATGAATGTCTTTTCACTACCCATTTGCTTATCCT | |
| | GCATCTCTCAGCCTTGACTCCACTCAGTTCTCTTGCTTAGAGATACC | |
| | ACCTTTCCCCTGAAGTGTTCCTTCCATGTTTTACGGCGAGATGGTTT | |
| | CTCCTCGCCTGGCCACTCAGCCTTAGTTGTCTCTGTTGTCTTATAGA | |
| | GGTCTACTTGAAGAAGGAAAAACAGGGGGCATGGTTTGACTGTCC | |
| | TGTGAGCCCTTCTTCCCTGCCTCCCCCACTCACAGTGACCCGGAAT | |
| | CCCTCGACATGGCAGTCTAGATCATTCTTGAAGACGAAAGGGCCTC | |
| | GTGATACGCCTATTTTTATAGGTTAATGTCATGATAATAATGGTTTC | |
| | TTAGACGTCAGGTGGCACTTTTCGGGGAAATGTGCGCGGAACCCCT | |
| | ATTTGTTTATTTTTCTAAATACATTCAAATATGTATCCGCTCATGAG | |
| | ACAATAACCCTGATAAATGCTTCAATAATATTGAAAAAGGAAGAG | |
| | TATGAGTATTCAACATTTCCGTGTCGCCCTTATTCCCTTTTTTGCGG | |
| | CATTTTGCCTTCCTGTTTTTGCTCACCCAGAAACGCTGGTGAAAGT | |
| | AAAAGATGCTGAAGATCAGTTGGGTGCACGAGTGGGTTACATCGA | |
| | ACTGGATCTCAACAGCGGTAAGATCCTTGAGAGTTTTCGCCCCGAA | |
| | GAACGTTTTCCAATGATGAGCACTTTTAAAGTTCTGCTATGTGGCG | |
| | CGGTATTATCCCGTATTGACGCCGGGCAAGAGCAACTCGGTCGCC | |
| | GCATACACTATTCTCAGAATGACTTGGTTGAGTACTCACCAGTCAC | |
| | AGAAAAGCATCTTACGGATGGCATGACAGTAAGAGAATTATGCAG | |
| | TGCTGCCATAACCATGAGTGATAACACTGCGGCCAACTTACTTCTG | |
| | ACAACGATCGGAGGACCGAAGGAGCTAACCGCTTTTTTGCACAAC | |
| | ATGGGGGATCATGTAACTCGCCTTGATCGTTGGGAACCGGAGCTG | |
| | AATGAAGCCATACCAAACGACGAGCGTGACACCACGATGCCTGTA | |
| | GCAATGGCAACAACGTTGCGCAAACTATTAACTGGCGAACTACTT | |
| | ACTCTAGCTTCCCGGCAACAATTAATAGACTGGATGGAGGCGGAT | |
| | AAAGTTGCAGGACCACTTCTGCGCTCGGCCCTTCCGGCTGGCTGGT | |
| | TTATTGCTGATAAATCTGGAGCCGGTGAGCGTGGGTCTCGCGGTAT | |
| | CATTGCAGCACTGGGGCCAGATGGTAAGCCCTCCCGTATCGTAGTT | |
| | ATCTACACGACGGGGAGTCAGGCAACTATGGATGAACGAAATAGA | |
| | CAGATCGCTGAGATAGGTGCCTCACTGATTAAGCATTGGTAACTGT | |
| | CAGACCAAGTTTACTCATATATACTTTAGATTGATTTAAAACTTCA | |
| | TTTTTAATTTAAAAGGATCTAGGTGAAGATCCTTTTTGATAATCTCA | |
| | TGACCAAAATCCCTTAACGTGAGTTTTCGTTCCACTGAGCGTCAGA | |
| | CCCCGTAGAAAAGATCAAAGGATCTTCTTGAGATCCTTTTTTTCTG | |
| | CGCGTAATCTGCTGCTTGCAAACAAAAAAACCACCGCTACCAGCG | |
| | GTGGTTTGTTTGCCGGATCAAGAGCTACCAACTCTTTTTCCGAAGG | |
| | TAACTGGCTTCAGCAGAGCGCAGATACCAAATACTGTTCTTCTAGT | |
| | GTAGCCGTAGTTAGGCCACCACTTCAAGAACTCTGTAGCACCGCCT | |
| | ACATACCTCGCTCTGCTAATCCTGTTACCAGTGGCTGCTGCCAGTG | |
| | GCGATAAGTCGTGTCTTACCGGGTTGGACTCAAGACGATAGTTACC | |
| | GGATAAGGCGCAGCGGTCGGGCTGAACGGGGGGTTCGTGCACACA | |
| | GCCCAGCTTGGAGCGAACGACCTACACCGAACTGAGATACCTACA | |
| | GCGTGAGCTATGAGAAAGCGCCACGCTTCCCGAAGGGAGAAAGGC | |
| | GGACAGGTATCCGGTAAGCGGCAGGGTCGGAACAGGAGAGCGCA | |
| | CGAGGGAGCTTCCAGGGGGAAACGCCTGGTATCTTTATAGTCCTGT | |
| | CGGGTTTCGCCACCTCTGACTTGAGCGTCGATTTTTGTGATGCTCGT | |
| | CAGGGGGGCGGAGCCTATGGAAAAACGCCAGCAACGGATGCGCC | |
| | GCGTGCGGCTGCTGGAGATGGCGGACGCGATGGATATGTTCTGCC | |
| | AAGGGTTGGTTTGCGCATTCACAGTTCTCCGCAAGAATTGATTGGC | |
| | TCCAATTCTTGGAGTGGTGAATCCGTTAGCGAGGTGCCGCCGGCTT | |
| | CCATTCAGGTCGAGGTGGCCCGGCTCCATGCACCGCGACGCAACG | |
| | CGGGGAGGCAGACAAGGTATAGGGCGGCGCCTACAATCCATGCCA | |
| | ACCCGTTCCATGTGCTCGCCGAGGCGGCATAAATCCCCGTGACGAT | |
| | CAGCGGTCCAATGATCGAAGTTAGGCTGGTAAGAGCCGCGAGCGA | |
| | TCCTTGAAGCTGTCCCTGATGGTCGTCATCTACCTGCCTGGACAGC | |
| | ATGGCCTGCAACGCGGGCATCCCGATGCCGCCGGAAGCGAGAAGA | |
| | ATCATAATGGGGAAGGCCATCCAGCCTCGCGTCGGGGAGCTTTTTG | |
| | CAAAAGCCTAGGCCTCCAAAAAAGCCTCCTCACTACTTCTGGAATA | |
| | GCTCAGAGGCCGAGGCGGCCTCGGCCTCTGCATAAATAAAAAAAA | |
| | TTAGTCAGCCATG | |
| 68 | AGCTTGGCCCATTGCATACGTTGTATCCATATCATAATATGTACAT | Triple Pal |
| | TTATATTGGCTCATGTCCAACATTACCGCCATGTTGACATTGATTAT | from GNA15 |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGCCCGCTCG | |
| | CTGACCTGGCGCTGCTGCCCCTGGTGCCTGACGGAGGATGAGAAG | |
| | GCCGCCGCCTCCCAGAACTACCCCATCGTGCAGATGTCCAATTTAC | |
| | TGACCGTACACCAAAATTTGCCTGCATTACCGGTCGATGCAACGAG | |
| | TGATGAGGTTCGCAAGAACCTGATGGACATGTTCAGGGATCGCCA | |
| | GGCGTTTTCTGAGCATACCTGGAAAATGCTTCTGTCCGTTTGCCGG | |
| | TCGTGGGCGGCATGGTGCAAGTTGAATAACCGGAAATGGTTTCCC | |
| | GCAGAACCTGAAGATGTTCGCGATTATCTTCTATATCTTCAGGCGC | |
| | GCGGTCTGGCAGTAAAAACTATCCAGCAACATTTGGGCCAGCTAA | |
| | ACATGCTTCATCGTCGGTCCGGGCTGCCACGACCAAGTGACAGCA | |
| | ATGCTGTTTCACTGGTTATGCGGCGGATCCGAAAAGAAAACGTTG | |
| | ATGCCGGTGAACGTGCAAAACAGGCTCTAGCGTTCGAACGCACTG | |
| | ATTTCGACCAGGTTCGTTCACTCATGGAAAATAGCGATCGCTGCCA | |
| | GGATATACGTAATCTGGCATTTCTGGGGATTGCTTATAACACCCTG | |
| | TTACGTATAGCCGAAATTGCCAGGATCAGGGTTAAAGATATCTCAC | |
| | GTACTGACGGTGGGAGAATGTTAATCCATATTGGCAGAACGAAAA | |
| | CGCTGGTTAGCACCGCAGGTGTAGAGAAGGCACTTAGCCTGGGGG | |
| | TAACTAAACTGGTCGAGCGATGGATTTCCGTCTCTGGTGTAGCTGA | |
| | TGATCCGAATAACTACCTGTTTTGCCGGGTCAGAAAAAATGGTGTT | |
| | GCCGCGCCATCTGCCACCAGCCAGCTATCAACTCGCGCCCTGGAA | |
| | GGGATTTTTGAAGCAACTCATCGATTGATTTACGGCGCTAAGGATG | |
| | ACTCTGGTCAGAGATACCTGGCCTGGTCTGGACACAGTGCCCGTGT | |
| | CGGAGCCGCGCGAGATATGGCCCGCGCTGGAGTTTCAATACCGGA | |
| | GATCATGCAAGCTGGTGGCTGGACCAATGTAAATATTGTCATGAA | |
| | CTATATCCGTAACCTGGATAGTGAAACAGGGGCAATGGTGCGCCT | |
| | GCTGGAAGATGGCGATTAAGGATCCAAGCTTATCGATACCGTCGA | |
| | CCTCGAGGGCCCAGATCTAATTCACCCCACCAGTGCAGGCTGCCTA | |
| | TCAGAAAGTGGTGGCTGGTGTGGCTAATGCCCTGGCCCACAAGTA | |
| | TCACTAAGCTCGCTTTCTTGCTGTCCAATTTCTATTAAAGGTTCCTT | |
| | TGTTCCCTAAGTCCAACTACTAAACTGGGGGATATTATGAAGGGCC | |
| | TTGAGCATCTGGATTCTGCCTAATAAAAAACATTTATTTTCATTGC | |
| | AATGATGTATTTAAATTATTTCTGAATATTTTACTAAAAAGGGAAT | |
| | GTGGGAGGTCAGTGCATTTAAAACATAAAGAAATGAAGAGCTAGT | |
| | TCAAACCTTGGGAAAATACACTATATCTTAAACTCCATGAAAGAA | |
| | GGTGAGGCTGCAAACAGCTAATGCACATTGGCAACAGCCCCTGAT | |
| | GCCTATGCCTTATTCATCCCTCAGAAAAGGATTCAAGTAGAGGCTT | |
| | GATTTGGAGGTTAAAGTTTTGCTATGCTGTATTTTACATTACTTATT | |
| | GTTTTAGCTGTCCTCATGAATGTCTTTTCACTACCCATTTGCTTATC | |
| | CTGCATCTCTCAGCCTTGACTCCACTCAGTTCTCTTGCTTAGAGATA | |
| | CCACCTTTCCCCTGAAGTGTTCCTTCCATGTTTTACGGCGAGATGGT | |
| | TTCTCCTCGCCTGGCCACTCAGCCTTAGTTGTCTCTGTTGTCTTATA | |
| | GAGGTCTACTTGAAGAAGGAAAAACAGGGGGCATGGTTTGACTGT | |
| | CCTGTGAGCCCTTCTTCCCTGCCTCCCCCACTCACAGTGACCCGGA | |
| | ATCCCTCGACATGGCAGTCTAGATCATTCTTGAAGACGAAAGGGC | |
| | CTCGTGATACGCCTATTTTTATAGGTTAATGTCATGATAATAATGG | |
| | TTTCTTAGACGTCAGGTGGCACTTTTCGGGGAAATGTGCGCGGAAC | |
| | CCCTATTTGTTTATTTTTCTAAATACATTCAAATATGTATCCGCTCA | |
| | TGAGACAATAACCCTGATAAATGCTTCAATAATATTGAAAAAGGA | |
| | AGAGTATGAGTATTCAACATTTCCGTGTCGCCCTTATTCCCTTTTTT | |
| | GCGGCATTTTGCCTTCCTGTTTTTGCTCACCCAGAAACGCTGGTGA | |
| | AAGTAAAAGATGCTGAAGATCAGTTGGGTGCACGAGTGGGTTACA | |
| | TCGAACTGGATCTCAACAGCGGTAAGATCCTTGAGAGTTTTCGCCC | |
| | CGAAGAACGTTTTCCAATGATGAGCACTTTTAAAGTTCTGCTATGT | |
| | GGCGCGGTATTATCCCGTATTGACGCCGGGCAAGAGCAACTCGGT | |
| | CGCCGCATACACTATTCTCAGAATGACTTGGTTGAGTACTCACCAG | |
| | TCACAGAAAAGCATCTTACGGATGGCATGACAGTAAGAGAATTAT | |
| | GCAGTGCTGCCATAACCATGAGTGATAACACTGCGGCCAACTTACT | |
| | TCTGACAACGATCGGAGGACCGAAGGAGCTAACCGCTTTTTTGCA | |
| | CAACATGGGGGATCATGTAACTCGCCTTGATCGTTGGGAACCGGA | |
| | GCTGAATGAAGCCATACCAAACGACGAGCGTGACACCACGATGCC | |
| | TGTAGCAATGGCAACAACGTTGCGCAAACTATTAACTGGCGAACT | |
| | ACTTACTCTAGCTTCCCGGCAACAATTAATAGACTGGATGGAGGCG | |
| | GATAAAGTTGCAGGACCACTTCTGCGCTCGGCCCTTCCGGCTGGCT | |
| | GGTTTATTGCTGATAAATCTGGAGCCGGTGAGCGTGGGTCTCGCGG | |
| | TATCATTGCAGCACTGGGGCCAGATGGTAAGCCCTCCCGTATCGTA | |
| | GTTATCTACACGACGGGGAGTCAGGCAACTATGGATGAACGAAAT | |
| | AGACAGATCGCTGAGATAGGTGCCTCACTGATTAAGCATTGGTAA | |
| | CTGTCAGACCAAGTTTACTCATATATACTTTAGATTGATTTAAAAC | |
| | TTCATTTTTAATTTAAAAGGATCTAGGTGAAGATCCTTTTTGATAAT | |
| | CTCATGACCAAAATCCCTTAACGTGAGTTTTCGTTCCACTGAGCGT | |
| | CAGACCCCGTAGAAAAGATCAAAGGATCTTCTTGAGATCCTTTTTT | |
| | TCTGCGCGTAATCTGCTGCTTGCAAACAAAAAAACCACCGCTACCA | |
| | GCGGTGGTTTGTTTGCCGGATCAAGAGCTACCAACTCTTTTTCCGA | |
| | AGGTAACTGGCTTCAGCAGAGCGCAGATACCAAATACTGTTCTTCT | |
| | AGTGTAGCCGTAGTTAGGCCACCACTTCAAGAACTCTGTAGCACCG | |
| | CCTACATACCTCGCTCTGCTAATCCTGTTACCAGTGGCTGCTGCCA | |
| | GTGGCGATAAGTCGTGTCTTACCGGGTTGGACTCAAGACGATAGTT | |
| | ACCGGATAAGGCGCAGCGGTCGGGCTGAACGGGGGGTTCGTGCAC | |
| | ACAGCCCAGCTTGGAGCGAACGACCTACACCGAACTGAGATACCT | |
| | ACAGCGTGAGCTATGAGAAAGCGCCACGCTTCCCGAAGGGAGAAA | |
| | GGCGGACAGGTATCCGGTAAGCGGCAGGGTCGGAACAGGAGAGC | |
| | GCACGAGGGAGCTTCCAGGGGGAAACGCCTGGTATCTTTATAGTC | |
| | CTGTCGGGTTTCGCCACCTCTGACTTGAGCGTCGATTTTTGTGATGC | |
| | TCGTCAGGGGGGCGGAGCCTATGGAAAAACGCCAGCAACGGATGC | |
| | GCCGCGTGCGGCTGCTGGAGATGGCGGACGCGATGGATATGTTCT | |
| | GCCAAGGGTTGGTTTGCGCATTCACAGTTCTCCGCAAGAATTGATT | |
| | GGCTCCAATTCTTGGAGTGGTGAATCCGTTAGCGAGGTGCCGCCGG | |
| | CTTCCATTCAGGTCGAGGTGGCCCGGCTCCATGCACCGCGACGCAA | |
| | CGCGGGGAGGCAGACAAGGTATAGGGCGGCGCCTACAATCCATGC | |
| | CAACCCGTTCCATGTGCTCGCCGAGGCGGCATAAATCCCCGTGACG | |
| | ATCAGCGGTCCAATGATCGAAGTTAGGCTGGTAAGAGCCGCGAGC | |
| | GATCCTTGAAGCTGTCCCTGATGGTCGTCATCTACCTGCCTGGACA | |
| | GCATGGCCTGCAACGCGGGCATCCCGATGCCGCCGGAAGCGAGAA | |
| | GAATCATAATGGGGAAGGCCATCCAGCCTCGCGTCGGGGAGCTTT | |
| | TTGCAAAAGCCTAGGCCTCCAAAAAAGCCTCCTCACTACTTCTGGA | |
| | ATAGCTCAGAGGCCGAGGCGGCCTCGGCCTCTGCATAAATAAAAA | |
| | AAATTAGTCAGCCATG | |
| 69 | AGCTTGGCCCATTGCATACGTTGTATCCATATCATAATATGTACAT | Myr-pal (lyn), |
| | TTATATTGGCTCATGTCCAACATTACCGCCATGTTGACATTGATTAT | N-term |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCTGCATC | |
| | AAATCAAAGAGGAAGGATAATCTGAATGACGATGAATCCCAGAAC | |
| | TACCCCATCGTGCAGATGTCCAATTTACTGACCGTACACCAAAATT | |
| | TGCCTGCATTACCGGTCGATGCAACGAGTGATGAGGTTCGCAAGA | |
| | ACCTGATGGACATGTTCAGGGATCGCCAGGCGTTTTCTGAGCATAC | |
| | CTGGAAAATGCTTCTGTCCGTTTGCCGGTCGTGGGCGGCATGGTGC | |
| | AAGTTGAATAACCGGAAATGGTTTCCCGCAGAACCTGAAGATGTT | |
| | CGCGATTATCTTCTATATCTTCAGGCGCGCGGTCTGGCAGTAAAAA | |
| | CTATCCAGCAACATTTGGGCCAGCTAAACATGCTTCATCGTCGGTC | |
| | CGGGCTGCCACGACCAAGTGACAGCAATGCTGTTTCACTGGTTATG | |
| | CGGCGGATCCGAAAAGAAAACGTTGATGCCGGTGAACGTGCAAAA | |
| | CAGGCTCTAGCGTTCGAACGCACTGATTTCGACCAGGTTCGTTCAC | |
| | TCATGGAAAATAGCGATCGCTGCCAGGATATACGTAATCTGGCATT | |
| | TCTGGGGATTGCTTATAACACCCTGTTACGTATAGCCGAAATTGCC | |
| | AGGATCAGGGTTAAAGATATCTCACGTACTGACGGTGGGAGAATG | |
| | TTAATCCATATTGGCAGAACGAAAACGCTGGTTAGCACCGCAGGT | |
| | GTAGAGAAGGCACTTAGCCTGGGGGTAACTAAACTGGTCGAGCGA | |
| | TGGATTTCCGTCTCTGGTGTAGCTGATGATCCGAATAACTACCTGT | |
| | TTTGCCGGGTCAGAAAAAATGGTGTTGCCGCGCCATCTGCCACCAG | |
| | CCAGCTATCAACTCGCGCCCTGGAAGGGATTTTTGAAGCAACTCAT | |
| | CGATTGATTTACGGCGCTAAGGATGACTCTGGTCAGAGATACCTGG | |
| | CCTGGTCTGGACACAGTGCCCGTGTCGGAGCCGCGCGAGATATGG | |
| | CCCGCGCTGGAGTTTCAATACCGGAGATCATGCAAGCTGGTGGCT | |
| | GGACCAATGTAAATATTGTCATGAACTATATCCGTAACCTGGATAG | |
| | TGAAACAGGGGCAATGGTGCGCCTGCTGGAAGATGGCGATTAAGG | |
| | ATCCAAGCTTATCGATACCGTCGACCTCGAGGGCCCAGATCTAATT | |
| | CACCCCACCAGTGCAGGCTGCCTATCAGAAAGTGGTGGCTGGTGT | |
| | GGCTAATGCCCTGGCCCACAAGTATCACTAAGCTCGCTTTCTTGCT | |
| | GTCCAATTTCTATTAAAGGTTCCTTTGTTCCCTAAGTCCAACTACTA | |
| | AACTGGGGGATATTATGAAGGGCCTTGAGCATCTGGATTCTGCCTA | |
| | ATAAAAAACATTTATTTTCATTGCAATGATGTATTTAAATTATTTCT | |
| | GAATATTTTACTAAAAAGGGAATGTGGGAGGTCAGTGCATTTAAA | |
| | ACATAAAGAAATGAAGAGCTAGTTCAAACCTTGGGAAAATACACT | |
| | ATATCTTAAACTCCATGAAAGAAGGTGAGGCTGCAAACAGCTAAT | |
| | GCACATTGGCAACAGCCCCTGATGCCTATGCCTTATTCATCCCTCA | |
| | GAAAAGGATTCAAGTAGAGGCTTGATTTGGAGGTTAAAGTTTTGCT | |
| | ATGCTGTATTTTACATTACTTATTGTTTTAGCTGTCCTCATGAATGT | |
| | CTTTTCACTACCCATTTGCTTATCCTGCATCTCTCAGCCTTGACTCC | |
| | ACTCAGTTCTCTTGCTTAGAGATACCACCTTTCCCCTGAAGTGTTCC | |
| | TTCCATGTTTTACGGCGAGATGGTTTCTCCTCGCCTGGCCACTCAG | |
| | CCTTAGTTGTCTCTGTTGTCTTATAGAGGTCTACTTGAAGAAGGAA | |
| | AAACAGGGGGCATGGTTTGACTGTCCTGTGAGCCCTTCTTCCCTGC | |
| | CTCCCCCACTCACAGTGACCCGGAATCCCTCGACATGGCAGTCTAG | |
| | ATCATTCTTGAAGACGAAAGGGCCTCGTGATACGCCTATTTTTATA | |
| | GGTTAATGTCATGATAATAATGGTTTCTTAGACGTCAGGTGGCACT | |
| | TTTCGGGGAAATGTGCGCGGAACCCCTATTTGTTTATTTTTCTAAAT | |
| | ACATTCAAATATGTATCCGCTCATGAGACAATAACCCTGATAAATG | |
| | CTTCAATAATATTGAAAAAGGAAGAGTATGAGTATTCAACATTTCC | |
| | GTGTCGCCCTTATTCCCTTTTTTGCGGCATTTTGCCTTCCTGTTTTTG | |
| | CTCACCCAGAAACGCTGGTGAAAGTAAAAGATGCTGAAGATCAGT | |
| | TGGGTGCACGAGTGGGTTACATCGAACTGGATCTCAACAGCGGTA | |
| | AGATCCTTGAGAGTTTTCGCCCCGAAGAACGTTTTCCAATGATGAG | |
| | CACTTTTAAAGTTCTGCTATGTGGCGCGGTATTATCCCGTATTGAC | |
| | GCCGGGCAAGAGCAACTCGGTCGCCGCATACACTATTCTCAGAAT | |
| | GACTTGGTTGAGTACTCACCAGTCACAGAAAAGCATCTTACGGAT | |
| | GGCATGACAGTAAGAGAATTATGCAGTGCTGCCATAACCATGAGT | |
| | GATAACACTGCGGCCAACTTACTTCTGACAACGATCGGAGGACCG | |
| | AAGGAGCTAACCGCTTTTTTGCACAACATGGGGGATCATGTAACTC | |
| | GCCTTGATCGTTGGGAACCGGAGCTGAATGAAGCCATACCAAACG | |
| | ACGAGCGTGACACCACGATGCCTGTAGCAATGGCAACAACGTTGC | |
| | GCAAACTATTAACTGGCGAACTACTTACTCTAGCTTCCCGGCAACA | |
| | ATTAATAGACTGGATGGAGGCGGATAAAGTTGCAGGACCACTTCT | |
| | GCGCTCGGCCCTTCCGGCTGGCTGGTTTATTGCTGATAAATCTGGA | |
| | GCCGGTGAGCGTGGGTCTCGCGGTATCATTGCAGCACTGGGGCCA | |
| | GATGGTAAGCCCTCCCGTATCGTAGTTATCTACACGACGGGGAGTC | |
| | AGGCAACTATGGATGAACGAAATAGACAGATCGCTGAGATAGGTG | |
| | CCTCACTGATTAAGCATTGGTAACTGTCAGACCAAGTTTACTCATA | |
| | TATACTTTAGATTGATTTAAAACTTCATTTTTAATTTAAAAGGATCT | |
| | AGGTGAAGATCCTTTTTGATAATCTCATGACCAAAATCCCTTAACG | |
| | TGAGTTTTCGTTCCACTGAGCGTCAGACCCCGTAGAAAAGATCAAA | |
| | GGATCTTCTTGAGATCCTTTTTTTCTGCGCGTAATCTGCTGCTTGCA | |
| | AACAAAAAAACCACCGCTACCAGCGGTGGTTTGTTTGCCGGATCA | |
| | AGAGCTACCAACTCTTTTTCCGAAGGTAACTGGCTTCAGCAGAGCG | |
| | CAGATACCAAATACTGTTCTTCTAGTGTAGCCGTAGTTAGGCCACC | |
| | ACTTCAAGAACTCTGTAGCACCGCCTACATACCTCGCTCTGCTAAT | |
| | CCTGTTACCAGTGGCTGCTGCCAGTGGCGATAAGTCGTGTCTTACC | |
| | GGGTTGGACTCAAGACGATAGTTACCGGATAAGGCGCAGCGGTCG | |
| | GGCTGAACGGGGGGTTCGTGCACACAGCCCAGCTTGGAGCGAACG | |
| | ACCTACACCGAACTGAGATACCTACAGCGTGAGCTATGAGAAAGC | |
| | GCCACGCTTCCCGAAGGGAGAAAGGCGGACAGGTATCCGGTAAGC | |
| | GGCAGGGTCGGAACAGGAGAGCGCACGAGGGAGCTTCCAGGGGG | |
| | AAACGCCTGGTATCTTTATAGTCCTGTCGGGTTTCGCCACCTCTGA | |
| | CTTGAGCGTCGATTTTTGTGATGCTCGTCAGGGGGGCGGAGCCTAT | |
| | GGAAAAACGCCAGCAACGGATGCGCCGCGTGCGGCTGCTGGAGAT | |
| | GGCGGACGCGATGGATATGTTCTGCCAAGGGTTGGTTTGCGCATTC | |
| | ACAGTTCTCCGCAAGAATTGATTGGCTCCAATTCTTGGAGTGGTGA | |
| | ATCCGTTAGCGAGGTGCCGCCGGCTTCCATTCAGGTCGAGGTGGCC | |
| | CGGCTCCATGCACCGCGACGCAACGCGGGGAGGCAGACAAGGTAT | |
| | AGGGCGGCGCCTACAATCCATGCCAACCCGTTCCATGTGCTCGCCG | |
| | AGGCGGCATAAATCCCCGTGACGATCAGCGGTCCAATGATCGAAG | |
| | TTAGGCTGGTAAGAGCCGCGAGCGATCCTTGAAGCTGTCCCTGATG | |
| | GTCGTCATCTACCTGCCTGGACAGCATGGCCTGCAACGCGGGCATC | |
| | CCGATGCCGCCGGAAGCGAGAAGAATCATAATGGGGAAGGCCATC | |
| | CAGCCTCGCGTCGGGGAGCTTTTTGCAAAAGCCTAGGCCTCCAAA | |
| | AAAGCCTCCTCACTACTTCTGGAATAGCTCAGAGGCCGAGGCGGC | |
| | CTCGGCCTCTGCATAAATAAAAAAAATTAGTCAGCCATG | |
| 70 | AGCTTGGCCCATTGCATACGTTGTATCCATATCATAATATGTACAT | Farnesyl, c- |
| | TTATATTGGCTCATGTCCAACATTACCGCCATGTTGACATTGATTAT | term, from |
| | TGACTAGTTATTAATAGTAATCAATTACGGGGTCATTAGTTCATAG | HRAS |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGTCCAATTTAC | |
| | TGACCGTACACCAAAATTTGCCTGCATTACCGGTCGATGCAACGAG | |
| | TGATGAGGTTCGCAAGAACCTGATGGACATGTTCAGGGATCGCCA | |
| | GGCGTTTTCTGAGCATACCTGGAAAATGCTTCTGTCCGTTTGCCGG | |
| | TCGTGGGCGGCATGGTGCAAGTTGAATAACCGGAAATGGTTTCCC | |
| | GCAGAACCTGAAGATGTTCGCGATTATCTTCTATATCTTCAGGCGC | |
| | GCGGTCTGGCAGTAAAAACTATCCAGCAACATTTGGGCCAGCTAA | |
| | ACATGCTTCATCGTCGGTCCGGGCTGCCACGACCAAGTGACAGCA | |
| | ATGCTGTTTCACTGGTTATGCGGCGGATCCGAAAAGAAAACGTTG | |
| | ATGCCGGTGAACGTGCAAAACAGGCTCTAGCGTTCGAACGCACTG | |
| | ATTTCGACCAGGTTCGTTCACTCATGGAAAATAGCGATCGCTGCCA | |
| | GGATATACGTAATCTGGCATTTCTGGGGATTGCTTATAACACCCTG | |
| | TTACGTATAGCCGAAATTGCCAGGATCAGGGTTAAAGATATCTCAC | |
| | GTACTGACGGTGGGAGAATGTTAATCCATATTGGCAGAACGAAAA | |
| | CGCTGGTTAGCACCGCAGGTGTAGAGAAGGCACTTAGCCTGGGGG | |
| | TAACTAAACTGGTCGAGCGATGGATTTCCGTCTCTGGTGTAGCTGA | |
| | TGATCCGAATAACTACCTGTTTTGCCGGGTCAGAAAAAATGGTGTT | |
| | GCCGCGCCATCTGCCACCAGCCAGCTATCAACTCGCGCCCTGGAA | |
| | GGGATTTTTGAAGCAACTCATCGATTGATTTACGGCGCTAAGGATG | |
| | ACTCTGGTCAGAGATACCTGGCCTGGTCTGGACACAGTGCCCGTGT | |
| | CGGAGCCGCGCGAGATATGGCCCGCGCTGGAGTTTCAATACCGGA | |
| | GATCATGCAAGCTGGTGGCTGGACCAATGTAAATATTGTCATGAA | |
| | CTATATCCGTAACCTGGATAGTGAAACAGGGGCAATGGTGCGCCT | |
| | GCTGGAAGATGGCGATTCCCAGAACTACCCCATCGTGCAGAAGCT | |
| | GAACCCTCCTGATGAGAGTGGCCCCGGCTGCATGAGCTGCAAGTG | |
| | TGTGCTCTCCTGATAAGGATCCAAGCTTATCGATACCGTCGACCTC | |
| | GAGGGCCCAGATCTAATTCACCCCACCAGTGCAGGCTGCCTATCA | |
| | GAAAGTGGTGGCTGGTGTGGCTAATGCCCTGGCCCACAAGTATCA | |
| | CTAAGCTCGCTTTCTTGCTGTCCAATTTCTATTAAAGGTTCCTTTGT | |
| | TCCCTAAGTCCAACTACTAAACTGGGGGATATTATGAAGGGCCTTG | |
| | AGCATCTGGATTCTGCCTAATAAAAAACATTTATTTTCATTGCAAT | |
| | GATGTATTTAAATTATTTCTGAATATTTTACTAAAAAGGGAATGTG | |
| | GGAGGTCAGTGCATTTAAAACATAAAGAAATGAAGAGCTAGTTCA | |
| | AACCTTGGGAAAATACACTATATCTTAAACTCCATGAAAGAAGGT | |
| | GAGGCTGCAAACAGCTAATGCACATTGGCAACAGCCCCTGATGCC | |
| | TATGCCTTATTCATCCCTCAGAAAAGGATTCAAGTAGAGGCTTGAT | |
| | TTGGAGGTTAAAGTTTTGCTATGCTGTATTTTACATTACTTATTGTT | |
| | TTAGCTGTCCTCATGAATGTCTTTTCACTACCCATTTGCTTATCCTG | |
| | CATCTCTCAGCCTTGACTCCACTCAGTTCTCTTGCTTAGAGATACCA | |
| | CCTTTCCCCTGAAGTGTTCCTTCCATGTTTTACGGCGAGATGGTTTC | |
| | TCCTCGCCTGGCCACTCAGCCTTAGTTGTCTCTGTTGTCTTATAGAG | |
| | GTCTACTTGAAGAAGGAAAAACAGGGGGCATGGTTTGACTGTCCT | |
| | GTGAGCCCTTCTTCCCTGCCTCCCCCACTCACAGTGACCCGGAATC | |
| | CCTCGACATGGCAGTCTAGATCATTCTTGAAGACGAAAGGGCCTC | |
| | GTGATACGCCTATTTTTATAGGTTAATGTCATGATAATAATGGTTTC | |
| | TTAGACGTCAGGTGGCACTTTTCGGGGAAATGTGCGCGGAACCCCT | |
| | ATTTGTTTATTTTTCTAAATACATTCAAATATGTATCCGCTCATGAG | |
| | ACAATAACCCTGATAAATGCTTCAATAATATTGAAAAAGGAAGAG | |
| | TATGAGTATTCAACATTTCCGTGTCGCCCTTATTCCCTTTTTTGCGG | |
| | CATTTTGCCTTCCTGTTTTTGCTCACCCAGAAACGCTGGTGAAAGT | |
| | AAAAGATGCTGAAGATCAGTTGGGTGCACGAGTGGGTTACATCGA | |
| | ACTGGATCTCAACAGCGGTAAGATCCTTGAGAGTTTTCGCCCCGAA | |
| | GAACGTTTTCCAATGATGAGCACTTTTAAAGTTCTGCTATGTGGCG | |
| | CGGTATTATCCCGTATTGACGCCGGGCAAGAGCAACTCGGTCGCC | |
| | GCATACACTATTCTCAGAATGACTTGGTTGAGTACTCACCAGTCAC | |
| | AGAAAAGCATCTTACGGATGGCATGACAGTAAGAGAATTATGCAG | |
| | TGCTGCCATAACCATGAGTGATAACACTGCGGCCAACTTACTTCTG | |
| | ACAACGATCGGAGGACCGAAGGAGCTAACCGCTTTTTTGCACAAC | |
| | ATGGGGGATCATGTAACTCGCCTTGATCGTTGGGAACCGGAGCTG | |
| | AATGAAGCCATACCAAACGACGAGCGTGACACCACGATGCCTGTA | |
| | GCAATGGCAACAACGTTGCGCAAACTATTAACTGGCGAACTACTT | |
| | ACTCTAGCTTCCCGGCAACAATTAATAGACTGGATGGAGGCGGAT | |
| | AAAGTTGCAGGACCACTTCTGCGCTCGGCCCTTCCGGCTGGCTGGT | |
| | TTATTGCTGATAAATCTGGAGCCGGTGAGCGTGGGTCTCGCGGTAT | |
| | CATTGCAGCACTGGGGCCAGATGGTAAGCCCTCCCGTATCGTAGTT | |
| | ATCTACACGACGGGGAGTCAGGCAACTATGGATGAACGAAATAGA | |
| | CAGATCGCTGAGATAGGTGCCTCACTGATTAAGCATTGGTAACTGT | |
| | CAGACCAAGTTTACTCATATATACTTTAGATTGATTTAAAACTTCA | |
| | TTTTTAATTTAAAAGGATCTAGGTGAAGATCCTTTTTGATAATCTCA | |
| | TGACCAAAATCCCTTAACGTGAGTTTTCGTTCCACTGAGCGTCAGA | |
| | CCCCGTAGAAAAGATCAAAGGATCTTCTTGAGATCCTTTTTTTCTG | |
| | CGCGTAATCTGCTGCTTGCAAACAAAAAAACCACCGCTACCAGCG | |
| | GTGGTTTGTTTGCCGGATCAAGAGCTACCAACTCTTTTTCCGAAGG | |
| | TAACTGGCTTCAGCAGAGCGCAGATACCAAATACTGTTCTTCTAGT | |
| | GTAGCCGTAGTTAGGCCACCACTTCAAGAACTCTGTAGCACCGCCT | |
| | ACATACCTCGCTCTGCTAATCCTGTTACCAGTGGCTGCTGCCAGTG | |
| | GCGATAAGTCGTGTCTTACCGGGTTGGACTCAAGACGATAGTTACC | |
| | GGATAAGGCGCAGCGGTCGGGCTGAACGGGGGGTTCGTGCACACA | |
| | GCCCAGCTTGGAGCGAACGACCTACACCGAACTGAGATACCTACA | |
| | GCGTGAGCTATGAGAAAGCGCCACGCTTCCCGAAGGGAGAAAGGC | |
| | GGACAGGTATCCGGTAAGCGGCAGGGTCGGAACAGGAGAGCGCA | |
| | CGAGGGAGCTTCCAGGGGGAAACGCCTGGTATCTTTATAGTCCTGT | |
| | CGGGTTTCGCCACCTCTGACTTGAGCGTCGATTTTTGTGATGCTCGT | |
| | CAGGGGGGCGGAGCCTATGGAAAAACGCCAGCAACGGATGCGCC | |
| | GCGTGCGGCTGCTGGAGATGGCGGACGCGATGGATATGTTCTGCC | |
| | AAGGGTTGGTTTGCGCATTCACAGTTCTCCGCAAGAATTGATTGGC | |
| | TCCAATTCTTGGAGTGGTGAATCCGTTAGCGAGGTGCCGCCGGCTT | |
| | CCATTCAGGTCGAGGTGGCCCGGCTCCATGCACCGCGACGCAACG | |
| | CGGGGAGGCAGACAAGGTATAGGGCGGCGCCTACAATCCATGCCA | |
| | ACCCGTTCCATGTGCTCGCCGAGGCGGCATAAATCCCCGTGACGAT | |
| | CAGCGGTCCAATGATCGAAGTTAGGCTGGTAAGAGCCGCGAGCGA | |
| | TCCTTGAAGCTGTCCCTGATGGTCGTCATCTACCTGCCTGGACAGC | |
| | ATGGCCTGCAACGCGGGCATCCCGATGCCGCCGGAAGCGAGAAGA | |
| | ATCATAATGGGGAAGGCCATCCAGCCTCGCGTCGGGGAGCTTTTTG | |
| | CAAAAGCCTAGGCCTCCAAAAAAGCCTCCTCACTACTTCTGGAATA | |
| | GCTCAGAGGCCGAGGCGGCCTCGGCCTCTGCATAAATAAAAAAAA | |
| | TTAGTCAGCCATG | |
| 71 | AGCTTGGCCCATTGCATACGTTGTATCCATATCATAATATGTACAT | HIV MA (N- |
| | TTATATTGGCTCATGTCCAACATTACCGCCATGTTGACATTGATTAT | term) |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCCGC | |
| | GCCTCCGTGCTGTCCGGCGGCGAGCTGGACAAGTGGGAGAAGATC | |
| | CGCCTGCGCCCCGGCGGCAAGAAGCAGTACAAGCTGAAGCACATC | |
| | GTGTGGGCCTCCCGCGAGCTGGAGCGCTTCGCCGTGAACCCCGGC | |
| | CTGCTGGAAACCTCCGAGGGCTGCCGCCAGATCCTGGGCCAGCTG | |
| | CAGCCCTCCCTGCAAACCGGCTCCGAGGAGCTGCGCTCCCTGTACA | |
| | ACACCATCGCCGTGCTGTACTGCGTGCACCAGCGCATCGACGTGA | |
| | AGGACACCAAGGAGGCCCTGGACAAGATCGAGGAGGAGCAGAAC | |
| | AAGTCCAAGAAGAAGGCCCAGCAGGCCGCCGCCGACACCGGCAA | |
| | CAACTCCCAGGTGTCCCAGAACTACCCCATCGTGCAGATGTCCAAT | |
| | TTACTGACCGTACACCAAAATTTGCCTGCATTACCGGTCGATGCAA | |
| | CGAGTGATGAGGTTCGCAAGAACCTGATGGACATGTTCAGGGATC | |
| | GCCAGGCGTTTTCTGAGCATACCTGGAAAATGCTTCTGTCCGTTTG | |
| | CCGGTCGTGGGCGGCATGGTGCAAGTTGAATAACCGGAAATGGTT | |
| | TCCCGCAGAACCTGAAGATGTTCGCGATTATCTTCTATATCTTCAG | |
| | GCGCGCGGTCTGGCAGTAAAAACTATCCAGCAACATTTGGGCCAG | |
| | CTAAACATGCTTCATCGTCGGTCCGGGCTGCCACGACCAAGTGACA | |
| | GCAATGCTGTTTCACTGGTTATGCGGCGGATCCGAAAAGAAAACG | |
| | TTGATGCCGGTGAACGTGCAAAACAGGCTCTAGCGTTCGAACGCA | |
| | CTGATTTCGACCAGGTTCGTTCACTCATGGAAAATAGCGATCGCTG | |
| | CCAGGATATACGTAATCTGGCATTTCTGGGGATTGCTTATAACACC | |
| | CTGTTACGTATAGCCGAAATTGCCAGGATCAGGGTTAAAGATATCT | |
| | CACGTACTGACGGTGGGAGAATGTTAATCCATATTGGCAGAACGA | |
| | AAACGCTGGTTAGCACCGCAGGTGTAGAGAAGGCACTTAGCCTGG | |
| | GGGTAACTAAACTGGTCGAGCGATGGATTTCCGTCTCTGGTGTAGC | |
| | TGATGATCCGAATAACTACCTGTTTTGCCGGGTCAGAAAAAATGGT | |
| | GTTGCCGCGCCATCTGCCACCAGCCAGCTATCAACTCGCGCCCTGG | |
| | AAGGGATTTTTGAAGCAACTCATCGATTGATTTACGGCGCTAAGGA | |
| | TGACTCTGGTCAGAGATACCTGGCCTGGTCTGGACACAGTGCCCGT | |
| | GTCGGAGCCGCGCGAGATATGGCCCGCGCTGGAGTTTCAATACCG | |
| | GAGATCATGCAAGCTGGTGGCTGGACCAATGTAAATATTGTCATG | |
| | AACTATATCCGTAACCTGGATAGTGAAACAGGGGCAATGGTGCGC | |
| | CTGCTGGAAGATGGCGATTAAGGATCCAAGCTTATCGATACCGTC | |
| | GACCTCGAGGGCCCAGATCTAATTCACCCCACCAGTGCAGGCTGC | |
| | CTATCAGAAAGTGGTGGCTGGTGTGGCTAATGCCCTGGCCCACAA | |
| | GTATCACTAAGCTCGCTTTCTTGCTGTCCAATTTCTATTAAAGGTTC | |
| | CTTTGTTCCCTAAGTCCAACTACTAAACTGGGGGATATTATGAAGG | |
| | GCCTTGAGCATCTGGATTCTGCCTAATAAAAAACATTTATTTTCAT | |
| | TGCAATGATGTATTTAAATTATTTCTGAATATTTTACTAAAAAGGG | |
| | AATGTGGGAGGTCAGTGCATTTAAAACATAAAGAAATGAAGAGCT | |
| | AGTTCAAACCTTGGGAAAATACACTATATCTTAAACTCCATGAAAG | |
| | AAGGTGAGGCTGCAAACAGCTAATGCACATTGGCAACAGCCCCTG | |
| | ATGCCTATGCCTTATTCATCCCTCAGAAAAGGATTCAAGTAGAGGC | |
| | TTGATTTGGAGGTTAAAGTTTTGCTATGCTGTATTTTACATTACTTA | |
| | TTGTTTTAGCTGTCCTCATGAATGTCTTTTCACTACCCATTTGCTTA | |
| | TCCTGCATCTCTCAGCCTTGACTCCACTCAGTTCTCTTGCTTAGAGA | |
| | TACCACCTTTCCCCTGAAGTGTTCCTTCCATGTTTTACGGCGAGATG | |
| | GTTTCTCCTCGCCTGGCCACTCAGCCTTAGTTGTCTCTGTTGTCTTA | |
| | TAGAGGTCTACTTGAAGAAGGAAAAACAGGGGGCATGGTTTGACT | |
| | GTCCTGTGAGCCCTTCTTCCCTGCCTCCCCCACTCACAGTGACCCG | |
| | GAATCCCTCGACATGGCAGTCTAGATCATTCTTGAAGACGAAAGG | |
| | GCCTCGTGATACGCCTATTTTTATAGGTTAATGTCATGATAATAAT | |
| | GGTTTCTTAGACGTCAGGTGGCACTTTTCGGGGAAATGTGCGCGGA | |
| | ACCCCTATTTGTTTATTTTTCTAAATACATTCAAATATGTATCCGCT | |
| | CATGAGACAATAACCCTGATAAATGCTTCAATAATATTGAAAAAG | |
| | GAAGAGTATGAGTATTCAACATTTCCGTGTCGCCCTTATTCCCTTTT | |
| | TTGCGGCATTTTGCCTTCCTGTTTTTGCTCACCCAGAAACGCTGGTG | |
| | AAAGTAAAAGATGCTGAAGATCAGTTGGGTGCACGAGTGGGTTAC | |
| | ATCGAACTGGATCTCAACAGCGGTAAGATCCTTGAGAGTTTTCGCC | |
| | CCGAAGAACGTTTTCCAATGATGAGCACTTTTAAAGTTCTGCTATG | |
| | TGGCGCGGTATTATCCCGTATTGACGCCGGGCAAGAGCAACTCGG | |
| | TCGCCGCATACACTATTCTCAGAATGACTTGGTTGAGTACTCACCA | |
| | GTCACAGAAAAGCATCTTACGGATGGCATGACAGTAAGAGAATTA | |
| | TGCAGTGCTGCCATAACCATGAGTGATAACACTGCGGCCAACTTAC | |
| | TTCTGACAACGATCGGAGGACCGAAGGAGCTAACCGCTTTTTTGCA | |
| | CAACATGGGGGATCATGTAACTCGCCTTGATCGTTGGGAACCGGA | |
| | GCTGAATGAAGCCATACCAAACGACGAGCGTGACACCACGATGCC | |
| | TGTAGCAATGGCAACAACGTTGCGCAAACTATTAACTGGCGAACT | |
| | ACTTACTCTAGCTTCCCGGCAACAATTAATAGACTGGATGGAGGCG | |
| | GATAAAGTTGCAGGACCACTTCTGCGCTCGGCCCTTCCGGCTGGCT | |
| | GGTTTATTGCTGATAAATCTGGAGCCGGTGAGCGTGGGTCTCGCGG | |
| | TATCATTGCAGCACTGGGGCCAGATGGTAAGCCCTCCCGTATCGTA | |
| | GTTATCTACACGACGGGGAGTCAGGCAACTATGGATGAACGAAAT | |
| | AGACAGATCGCTGAGATAGGTGCCTCACTGATTAAGCATTGGTAA | |
| | CTGTCAGACCAAGTTTACTCATATATACTTTAGATTGATTTAAAAC | |
| | TTCATTTTTAATTTAAAAGGATCTAGGTGAAGATCCTTTTTGATAAT | |
| | CTCATGACCAAAATCCCTTAACGTGAGTTTTCGTTCCACTGAGCGT | |
| | CAGACCCCGTAGAAAAGATCAAAGGATCTTCTTGAGATCCTTTTTT | |
| | TCTGCGCGTAATCTGCTGCTTGCAAACAAAAAAACCACCGCTACCA | |
| | GCGGTGGTTTGTTTGCCGGATCAAGAGCTACCAACTCTTTTTCCGA | |
| | AGGTAACTGGCTTCAGCAGAGCGCAGATACCAAATACTGTTCTTCT | |
| | AGTGTAGCCGTAGTTAGGCCACCACTTCAAGAACTCTGTAGCACCG | |
| | CCTACATACCTCGCTCTGCTAATCCTGTTACCAGTGGCTGCTGCCA | |
| | GTGGCGATAAGTCGTGTCTTACCGGGTTGGACTCAAGACGATAGTT | |
| | ACCGGATAAGGCGCAGCGGTCGGGCTGAACGGGGGGTTCGTGCAC | |
| | ACAGCCCAGCTTGGAGCGAACGACCTACACCGAACTGAGATACCT | |
| | ACAGCGTGAGCTATGAGAAAGCGCCACGCTTCCCGAAGGGAGAAA | |
| | GGCGGACAGGTATCCGGTAAGCGGCAGGGTCGGAACAGGAGAGC | |
| | GCACGAGGGAGCTTCCAGGGGGAAACGCCTGGTATCTTTATAGTC | |
| | CTGTCGGGTTTCGCCACCTCTGACTTGAGCGTCGATTTTTGTGATGC | |
| | TCGTCAGGGGGGCGGAGCCTATGGAAAAACGCCAGCAACGGATGC | |
| | GCCGCGTGCGGCTGCTGGAGATGGCGGACGCGATGGATATGTTCT | |
| | GCCAAGGGTTGGTTTGCGCATTCACAGTTCTCCGCAAGAATTGATT | |
| | GGCTCCAATTCTTGGAGTGGTGAATCCGTTAGCGAGGTGCCGCCGG | |
| | CTTCCATTCAGGTCGAGGTGGCCCGGCTCCATGCACCGCGACGCAA | |
| | CGCGGGGAGGCAGACAAGGTATAGGGCGGCGCCTACAATCCATGC | |
| | CAACCCGTTCCATGTGCTCGCCGAGGCGGCATAAATCCCCGTGACG | |
| | ATCAGCGGTCCAATGATCGAAGTTAGGCTGGTAAGAGCCGCGAGC | |
| | GATCCTTGAAGCTGTCCCTGATGGTCGTCATCTACCTGCCTGGACA | |
| | GCATGGCCTGCAACGCGGGCATCCCGATGCCGCCGGAAGCGAGAA | |
| | GAATCATAATGGGGAAGGCCATCCAGCCTCGCGTCGGGGAGCTTT | |
| | TTGCAAAAGCCTAGGCCTCCAAAAAAGCCTCCTCACTACTTCTGGA | |
| | ATAGCTCAGAGGCCGAGGCGGCCTCGGCCTCTGCATAAATAAAAA | |
| | AAATTAGTCAGCCATG | |
| 72 | GARASVLSGGELDKWEKIRLRPGGKKQYKLKHIVWASRELERFAVNP | pMA2 N-term |
| | GLLETSEGCRQILGQLQPSLQTGSEELRSLYNTIAVLYCVHQRIDVKDT | GAG-T2A-Cre |
| | KEALDKIEEEQNKSKKKAQQAGSGEGRGSLLTCGDVEENPGPMVPKK | (no promoter; |
| | KRKVSNLLTVHQNLPALPVDATSDEVRKNLMDMFRDRQAFSEHTWK | no Met) |
| | MLLSVCRSWAAWCKLNNRKWFPAEPEDVRDYLLYLQARGLAVKTIQ | |
| | QHLGQLNMLHRRSGLPRPSDSNAVSLVMRRIRKENVDAGERAKQAL | |
| | AFERTDFDQVRSLMENSDRCQDIRNLAFLGIAYNTLLRIAEIARIRVKD | |
| | ISRTDGGRMLIHIGRTKTLVSTAGVEKALSLGVTKLVERWISVSGVAD | |
| | DPNNYLFCRVRKNGVAAPSATSQLSTRALEGIFEATHRLIYGAKDDSG | |
| | QRYLAWSGHSARVGAARDMARAGVSIPEIMQAGGWTNVNIVMNYIR | |
| | NLDSETGAMVRLLEDGD | |
| 73 | GARASVLSGGELDKWEKIRLRPGGKKQYKLKHIVWASRELERFAVNP | MA-MS2_{cp}- |
| | GLLETSEGCRQILGQLQPSLQTGSEELRSLYNTIAVLYCVHQRIDVKDT | CA gagpol (no |
| | KEALDKIEEEQNKSKKKAQQAAADTGNNSQVSQNYPIVQMASNFTQF | Met) |
| | VLVDNGGTGDVTVAPSNFANGIAEWISSNSRSQAYKVTCSVRQSSAQ | |
| | NRKYTIKVEVPKGAWRSYLNMELTIPIFATNSDCELIVKAMQGLLKD | |
| | GNPIPSAIAANSGIYSQNYPIVQNLQGQMVHQAISPRTLNAWVKVVEE | |
| | KAFSPEVIPMFSALSEGATPQDLNTMLNTVGGHQAAMQMLKETINEE | |
| | AAEWDRLHPVHAGPIAPGQMREPRGSDIAGTTSTLQEQIGWMTHNPPI | |
| | PVGEIYKRWIILGLNKIVRMYSPTSILDIRQGPKEPFRDYVDRFYKTLR | |
| | AEQASQEVKNWMTETLLVQNANPDCKTILKALGPGATLEEMMTACQ | |
| | GVGGPGHKARVLAEAMSQVTNPATIMIQKGNFRNQRKTVKCFNCGK | |
| | EGHIAKNCRAPRKKGCWKCGKEGHQMKDCTERQANFLGKIWPSHK | |
| | GRPGNFLQSRPEPTAPPEESFRFGEETTTPSQKQEPIDKELYPLASLRSL | |
| | FGSDPSSQ | |
| 74 | GARASVLSGGELDKWEKIRLRPGGKKQYKLKHIVWASRELERFAVNP | HIV MA (N- |
| | GLLETSEGCRQILGQLQPSLQTGSEELRSLYNTIAVLYCVHQRIDVKDT | term)-MS2_{cp} |
| | KEALDKIEEEQNKSKKKAQQAAADTGNNSQVSQNYPIVQMASNFTQF | (no Met) |
| | VLVDNGGTGDVTVAPSNFANGIAEWISSNSRSQAYKVTCSVRQSSAQ | |
| | NRKYTIKVEVPKGAWRSYLNMELTIPIFATNSDCELIVKAMQGLLKD | |
| | GNPIPSAIAANSGIY | |
| 75 | GARASVLSGGELDKWEKIRLRPGGKKQYKLKHIVWASRELERFAVNP | HIV MA (N- |
| | GLLETSEGCRQILGQLQPSLQTGSEELRSLYNTIAVLYCVHQRIDVKDT | term)-Cre (no |
| | KEALDKIEEEQNKSKKKAQQAAADTGNNSQVSQNYPIVQMSNLLTV | Met) |
| | HQNLPALPVDATSDEVRKNLMDMFRDRQAFSEHTWKMLLSVCRSW | |
| | AAWCKLNNRKWFPAEPEDVRDYLLYLQARGLAVKTIQQHLGQLNM | |
| | LHRRSGLPRPSDSNAVSLVMRRIRKENVDAGERAKQALAFERTDFDQ | |
| | VRSLMENSDRCQDIRNLAFLGIAYNTLLRIAEIARIRVKDISRTDGGRM | |
| | LIHIGRTKTLVSTAGVEKALSLGVTKLVERWISVSGVADDPNNYLFCR | |
| | VRKNGVAAPSATSQLSTRALEGIFEATHRLIYGAKDDSGQRYLAWSG | |
| | HSARVGAARDMARAGVSIPEIMQAGGWTNVNIVMNYIRNLDSETGA | |
| | MVRLLEDGD | |
| 76 | GSGEGRGSLLTCGDVEENPGP | T2A |
| 77 | GARASVLSGGELDKWEKIRLRPGGKKQYKLKHIVWASRELERFAVNP | gag-T2A (no |
| | GLLETSEGCRQILGQLQPSLQTGSEELRSLYNTIAVLYCVHQRIDVKDT | Met) |
| | KEALDKIEEEQNKSKKKAQQAGSGEGRGSLLTCGDVEENPGP | |
| 78 | GARASVLSGGELDKWEKIRLRPGGKKQYKLKHIVWASRELERFAVNP | HIV MA- |
| | GLLETSEGCRQILGQLQPSLQTGSEELRSLYNTIAVLYCVHQRIDVKDT | cleavage site |
| | KEALDKIEEEQNKSKKKAQQAAADTGNNSQVSQNYPIVQ | (no Met) |
| 79 | ASNFTQFVLVDNGGTGDVTVAPSNFANGIAEWISSNSRSQAYKVTCS | MS2_{cp} (no |
| | VRQSSAQNRKYTIKVEVPKGAWRSYLNMELTIPIFATNSDCELIVKAM | Met) |
| | QGLLKDGNPIPSAIAANSGIY | |
| 80 | QVQLVQSGAEVKKPGASVKVSCKASGGTFSSYAISWVRQAPGQGLE | CD8 scFv |
| | WMGIIDPSDGNTNYAQNFQGRVTMTRDTSTSTVYMELSSLRSEDTAV | |
| | YYCAKERAAAGYYYYMDVWGQGTTVTVSSGGGGSGGGGSGGGGS | |
| | DIQMTQSPSSLSASVGDRVTITCRASQSISSYLNWYQQKPGKAPKLLIY | |
| | AASSLQSGVPSRFSGSGSGTDFTLTISSLQPEDFATYYCQQSYSTPLTFG | |
| | GGTKVEIKR | |
| 81 | QVQLVQSGAEVKKPGASVKVSCKASGYTFTDYYIQWVRQAPGQGLE | CD8 scFv |
| | WMGWINPNSGGTSYAQKFQGRVTMTRDTSTSTVYMELSSLRSEDTA | |
| | VYYCAKEGDYYYGMDAWGQGTMVTVSSGGGGSGGGGSGGGGSDI | |
| | VMTQSPLSLPVTPGEPASISCRSSQSLLHSNGYNYLDWYLQKPGQSPQ | |
| | LLIYLGSNRASGVPDRFSGSGSGTDFTLKISRVEAEDVGVYYCMQGLQ | |
| | TPHTFGQGTKVEIKR | |
| 82 | QVQLVQSGAEVKKPGASVKVSCKASGYTFTSYYMHWVRQAPGQGL | CD8 scFv |
| | EWMGGFDPEDGETIYAQKFQGRVTMTRDTSTSTVYMELSSLRSEDTA | |
| | VYYCARDQGWGMDVWGQGTTVTVSSGGGGSGGGGSGGGGSDIQM | |
| | TQSPSSLSASVGDRVTITCRASQSISSYLNWYQQKPGKAPKLLIYAASS | |
| | LQSGVPSRFSGSGSGTDFTLTISSLQPEDFATYYCQQTYSTPYTFGQGT | |
| | KLEIKR | |
| 83 | QVQLVQSGAEVKKPGASVKVSCKASGYTFTNHYMHWVRQAPGQGL | CD8 scFv |
| | EWMGWMNPNSGNTGYAQKFQGRVTMTRDTSTSTVYMELSSLRSED | |
| | TAVYYCASSESGSDLDYWGQGTLVTVSSGGGGSGGGGSGGGGSDIQ | |
| | MTQSPSSLSASVGDRVTITCRASQTIGNYVNWYQQKPGKAPKLLIYG | |
| | ASNLHTGVPSRFSGSGSGTDFTLTISSLQPEDFATYYCQQTYSAPLTFG | |
| | GGTKVEIKR | |
| 84 | QVQLVESGGGLVQAGGSLRLSCAASGRTFSGYVMGWFRQAPGKQRK | CD8 VHH |
| | FVAAISRGGLSTSYADSVKGRFTISRDNAKNTVFLQMNTLKPEDTAVY | |
| | YCAADRSDLYEITAASNIDSWGQGTLVTVSS | |
| 85 | SYAIS | CDR-H1 |
| 86 | IIDPSDGNTNYAQNFQG | CDR-H2 |
| 87 | ERAAAGYYYYMDV | CDR-H3 |
| 88 | RASQSISSYLN | CDR-L1 |
| 89 | AASSLQS | CDR-L2 |
| 90 | QQSYSTPLT | CDR-L3 |
| 91 | QVQLVQSGAEVKKPGASVKVSCKASGGTFSSYAISWVRQAPGQGLE | VH |
| | WMGIIDPSDGNTNYAQNFQGRVTMTRDTSTSTVYMELSSLRSEDTAV | |
| | YYCAKERAAAGYYYYMDVWGQGTTVTVSS | |
| 92 | DIQMTQSPSSLSASVGDRVTITCRASQSISSYLNWYQQKPGKAPKLLIY | VL |
| | AASSLQSGVPSRFSGSGSGTDFTLTISSLQPEDFATYYCQQSYSTPLTFG | |
| | GGTKVEIKR | |
| 93 | DYYIQ | CDR-H1 |
| 94 | WINPNSGGTSYAQKFQG | CDR-H2 |
| 95 | EGDYYYGMDA | CDR-H3 |
| 96 | RSSQSLLHSNGYNYLD | CDR-L1 |
| 97 | LGSNRAS | CDR-L2 |
| 98 | MQGLQTPHT | CDR-L3 |
| 99 | QVQLVQSGAEVKKPGASVKVSCKASGYTFTDYYIQWVRQAPGQGLE | VH |
| | WMGWINPNSGGTSYAQKFQGRVTMTRDTSTSTVYMELSSLRSEDTA | |
| | VYYCAKEGDYYYGMDAWGQGTMVTVSS | |
| 100 | DIVMTQSPLSLPVTPGEPASISCRSSQSLLHSNGYNYLDWYLQKPGQSP | VL |
| | QLLIYLGSNRASGVPDRFSGSGSGTDFTLKISRVEAEDVGVYYCMQGL | |
| | QTPHTFGQGTKVEIKR | |
| 101 | SYYMH | CDR-H1 |
| 102 | GFDPEDGETIYAQKFQG | CDR-H2 |
| 103 | DQGWGMDV | CDR-H3 |
| 104 | QQTYSTPYT | CDR-L3 |
| 105 | QVQLVQSGAEVKKPGASVKVSCKASGYTFTSYYMHWVRQAPGQGL | VH |
| | EWMGGFDPEDGETIYAQKFQGRVTMTRDTSTSTVYMELSSLRSEDTA | |
| | VYYCARDQGWGMDVWGQGTTVTVSS | |
| 106 | DIQMTQSPSSLSASVGDRVTITCRASQSISSYLNWYQQKPGKAPKLLIY | VL |
| | AASSLQSGVPSRFSGSGSGTDFTLTISSLQPEDFATYYCQQTYSTPYTF | |
| | GQGTKLEIKR | |
| 107 | NHYMH | CDR-H1 |
| 108 | WMNPNSGNTGYAQKFQG | CDR-H2 |
| 109 | SESGSDLDY | CDR-H3 |
| 110 | RASQTIGNYVN | CDR-L1 |
| 111 | GASNLHT | CDR-L2 |
| 112 | QQTYSAPLT | CDR-L3 |
| 113 | QVQLVQSGAEVKKPGASVKVSCKASGYTFTNHYMHWVRQAPGQGL | VH |
| | EWMGWMNPNSGNTGYAQKFQGRVTMTRDTSTSTVYMELSSLRSED | |
| | TAVYYCASSESGSDLDYWGQGTLVTVSS | |
| 114 | DIQMTQSPSSLSASVGDRVTITCRASQTIGNYVNWYQQKPGKAPKLLI | VL |
| | YGASNLHTGVPSRFSGSGSGTDFTLTISSLQPEDFATYYCQQTYSAPLT | |
| | FGGGTKVEIKR | |
| 115 | GYVMG | CDR-H1 |
| 116 | AISRGGLSTSYADSVKG | CDR-H2 |
| 117 | DRSDLYEITAASNIDS | CDR-H3 |
| 118 | RYTMH | CD3 CDR-H1 |
| 119 | YINPSRGYTNYNQKFKD | CD3 CDR-H2 |
| 120 | YYDDHYCLDY | CD3 CDR-H3 |
| 121 | RASSSVSYMN | CD3 CDR-L1 |
| 122 | DTSKVAS | CD3 CDR-L2 |
| 123 | QQWSSNPLT | CD3 CDR-L3 |
| 124 | DIKLQQSGAELARPGASVKMSCKTSGYTFTRYTMHWVKQRPGQGLE | CD3 VH |
| | WIGYINPSRGYTNYNQKFKDKATLTTDKSSSTAYMQLSSLTSEDSAV | |
| | YYCARYYDDHYCLDYWGQGTTLTVSSVE | |
| 125 | DIQLTQSPAIMSASPGEKVTMTCRASSSVSYMNWYQQKSGTSPKRWI | CD3 VL |
| | YDTSKVASGVPYRFSGSGSGTSYSLTISSMEAEDAATYYCQQWSSNPL | |
| | TFGAGTKLELK | |
| 126 | DIKLQQSGAELARPGASVKMSCKTSGYTFTRYTMHWVKQRPGQGLE | CD3 scFv |
| | WIGYINPSRGYTNYNQKFKDKATLTTDKSSSTAYMQLSSLTSEDSAV | |
| | YYCARYYDDHYCLDYWGQGTTLTVSSVEGGSGGSGGSGGSGGVDDI | |
| | QLTQSPAIMSASPGEKVTMTCRASSSVSYMNWYQQKSGTSPKRWIYD | |
| | TSKVASGVPYRFSGSGSGTSYSLTISSMEAEDAATYYCQQWSSNPLTF | |
| | GAGTKLELK | |
| 127 | GARASVLSGGELDKWEKIRLRPGGKKQYKLKHIVWASRELERFAVNP | HIV MA (no |
| | GLLETSEGCRQILGQLQPSLQTGSEELRSLYNTIAVLYCVHQRIDVKDT | Met) |
| | KEALDKIEEEQNKSKKKAQQAAADTGNNSQVSQNY | |
| 128 | SQNYPIVQ | cleavage site |
| 129 | PIVQNLQGQMVHQAISPRTLNAWVKVVEEKAFSPEVIPMFSAL | CA |
| | SEGATPQDLNTMLNTVGGHQAAMQMLKETINEEAAEWDRLHPVHA | |
| | GPIAPGQMREPRGSDIAGTTSTLQEQIGWMTHNPPIPVGEIYKRWIILG | |
| | LNKIVRMYSPTSILDIRQGPKEPFRDYVDRFYKTLRAEQASQEVKNW | |
| | MTETLLVQNANPDCKTILKALGPGATLEEMMTACQGVGGPGHKARV | |
| | LAEAMSQVTNPATIMIQKGNFRNQRKTVKCFNCGKEGHIAKNCRAPR | |
| | KKGCWKCGKEGHQMKDCTERQANFLGKIWPSHKGRPGNFLQSRPEP | |
| | TAPPEESFRFGEETTTPSQKQEPIDKELYPLASLRSLFGSDPSSQ | |
| 130 | SQNYPIVQNLQGQMVHQAISPRTLNAWVKVVEEKAFSPEVIPMFSAL | CA-cleavage |
| | SEGATPQDLNTMLNTVGGHQAAMQMLKETINEEAAEWDRLHPVHA | site |
| | GPIAPGQMREPRGSDIAGTTSTLQEQIGWMTHNPPIPVGEIYKRWIILG | |
| | LNKIVRMYSPTSILDIRQGPKEPFRDYVDRFYKTLRAEQASQEVKNW | |
| | MTETLLVQNANPDCKTILKALGPGATLEEMMTACQGVGGPGHKARV | |
| | LAEAMSQVTNPATIMIQKGNFRNQRKTVKCFNCGKEGHIAKNCRAPR | |
| | KKGCWKCGKEGHQMKDCTERQANFLGKIWPSHKGRPGNFLQSRPEP | |
| | TAPPEESFRFGEETTTPSQKQEPIDKELYPLASLRSLFGSDPSSQ | |
| 131 | MGARASVLSGGELDKWEKIRLRPGGKKQYKLKHIVWASRELERFAV | N-term gag |
| | NPGLLETSEGCRQILGQLQPSLQTGSEELRSLYNTIAVLYCVHQRIDVK | |
| | DTKEALDKIEEEQNKSKKKAQQA | |
| 132 | MGARASVLSGGELDKWEKIRLRPGGKKQYKLKHIVWASRELERFAV | pMA2 N-term |
| | NPGLLETSEGCRQILGQLQPSLQTGSEELRSLYNTIAVLYCVHQRIDVK | GAG-T2A-Cre |
| | DTKEALDKIEEEQNKSKKKAQQAGSGEGRGSLLTCGDVEENPGPMVP | (no promoter) |
| | KKKRKVSNLLTVHQNLPALPVDATSDEVRKNLMDMFRDRQAFSEHT | |
| | WKMLLSVCRSWAAWCKLNNRKWFPAEPEDVRDYLLYLQARGLAVK | |
| | TIQQHLGQLNMLHRRSGLPRPSDSNAVSLVMRRIRKENVDAGERAKQ | |
| | ALAFERTDFDQVRSLMENSDRCQDIRNLAFLGIAYNTLLRIAEIARIRV | |
| | KDISRTDGGRMLIHIGRTKTLVSTAGVEKALSLGVTKLVERWISVSGV | |
| | ADDPNNYLFCRVRKNGVAAPSATSQLSTRALEGIFEATHRLIYGAKD | |
| | DSGQRYLAWSGHSARVGAARDMARAGVSIPEIMQAGGWTNVNIVM | |
| | NYIRNLDSETGAMVRLLEDGD | |
| 133 | MGARASVLSGGELDKWEKIRLRPGGKKQYKLKHIVWASRELERFAV | MA-MS2_{cp}- |
| | NPGLLETSEGCRQILGQLQPSLQTGSEELRSLYNTIAVLYCVHQRIDVK | CA gagpol |
| | DTKEALDKIEEEQNKSKKKAQQAAADTGNNSQVSQNYPIVQMASNF | |
| | TQFVLVDNGGTGDVTVAPSNFANGIAEWISSNSRSQAYKVTCSVRQS | |
| | SAQNRKYTIKVEVPKGAWRSYLNMELTIPIFATNSDCELIVKAMQGLL | |
| | KDGNPIPSAIAANSGIYSQNYPIVQNLQGQMVHQAISPRTLNAWVKVV | |
| | EEKAFSPEVIPMFSALSEGATPQDLNTMLNTVGGHQAAMQMLKETIN | |
| | EEAAEWDRLHPVHAGPIAPGQMREPRGSDIAGTTSTLQEQIGWMTHN | |
| | PPIPVGEIYKRWIILGLNKIVRMYSPTSILDIRQGPKEPFRDYVDRFYKT | |
| | LRAEQASQEVKNWMTETLLVQNANPDCKTILKALGPGATLEEMMTA | |
| | CQGVGGPGHKARVLAEAMSQVTNPATIMIQKGNFRNQRKTVKCFNC | |
| | GKEGHIAKNCRAPRKKGCWKCGKEGHQMKDCTERQANFLGKIWPS | |
| | HKGRPGNFLQSRPEPTAPPEESFRFGEETTTPSQKQEPIDKELYPLASLR | |
| | SLFGSDPSSQ | |
| 134 | MGARASVLSGGELDKWEKIRLRPGGKKQYKLKHIVWASRELERFAV | HIV MA (N- |
| | NPGLLETSEGCRQILGQLQPSLQTGSEELRSLYNTIAVLYCVHQRIDVK | term)-MS2_{cp} |
| | DTKEALDKIEEEQNKSKKKAQQAAADTGNNSQVSQNYPIVQMASNF | |
| | TQFVLVDNGGTGDVTVAPSNFANGIAEWISSNSRSQAYKVTCSVRQS | |
| | SAQNRKYTIKVEVPKGAWRSYLNMELTIPIFATNSDCELIVKAMQGLL | |
| | KDGNPIPSAIAANSGIY | |
| 135 | MGARASVLSGGELDKWEKIRLRPGGKKQYKLKHIVWASRELERFAV | HIV MA (N- |
| | NPGLLETSEGCRQILGQLQPSLQTGSEELRSLYNTIAVLYCVHQRIDVK | term)-Cre |
| | DTKEALDKIEEEQNKSKKKAQQAAADTGNNSQVSQNYPIVQMSNLLT | |
| | VHQNLPALPVDATSDEVRKNLMDMFRDRQAFSEHTWKMLLSVCRS | |
| | WAAWCKLNNRKWFPAEPEDVRDYLLYLQARGLAVKTIQQHLGQLN | |
| | MLHRRSGLPRPSDSNAVSLVMRRIRKENVDAGERAKQALAFERTDFD | |
| | QVRSLMENSDRCQDIRNLAFLGIAYNTLLRIAEIARIRVKDISRTDGGR | |
| | MLIHIGRTKTLVSTAGVEKALSLGVTKLVERWISVSGVADDPNNYLFC | |
| | RVRKNGVAAPSATSQLSTRALEGIFEATHRLIYGAKDDSGQRYLAWS | |
| | GHSARVGAARDMARAGVSIPEIMQAGGWTNVNIVMNYIRNLDSETG | |
| | AMVRLLEDGD | |
| 136 | MGARASVLSGGELDKWEKIRLRPGGKKQYKLKHIVWASRELERFAV | gag-T2A |
| | NPGLLETSEGCRQILGQLQPSLQTGSEELRSLYNTIAVLYCVHQRIDVK | |
| | DTKEALDKIEEEQNKSKKKAQQAGSGEGRGSLLTCGDVEENPGP | |
| 137 | MGARASVLSGGELDKWEKIRLRPGGKKQYKLKHIVWASRELERFAV | HIV MA- |
| | NPGLLETSEGCRQILGQLQPSLQTGSEELRSLYNTIAVLYCVHQRIDVK | cleavage site |
| | DTKEALDKIEEEQNKSKKKAQQAAADTGNNSQVSQNYPIVQ | |
| 138 | MASNFTQFVLVDNGGTGDVTVAPSNFANGIAEWISSNSRSQAYKVTC | MS2_{cp} |
| | SVRQSSAQNRKYTIKVEVPKGAWRSYLNMELTIPIFATNSDCELIVKA | |
| | MQGLLKDGNPIPSAIAANSGIY | |
| 139 | MGARASVLSGGELDKWEKIRLRPGGKKQYKLKHIVWASRELERFAV | HIV MA |
| | NPGLLETSEGCRQILGQLQPSLQTGSEELRSLYNTIAVLYCVHQRIDVK | |
| | DTKEALDKIEEEQNKSKKKAQQAAADTGNNSQVSQNY | |
| 140 | PIVQ | cleavage site |
| 141 | TGGAAGGGCTAATTCACTCCCAAAGAAGACAAGATATCCTTGATC | N-term |
| | TGTGGATCTACCACACACAAGGCTACTTCCCTGATTAGCAGAACTA | mutated MSD |
| | CACACCAGGGCCAGGGGTCAGATATCCACTGACCTTTGGATGGTG | GAG-T2A-Cre |
| | CTACAAGCTAGTACCAGTTGAGCCAGATAAGGTAGAAGAGGCCAA | |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGCAGAGTACGCCAAAAATTTTGACTAGCGG | |
| | AGGCTAGAAGGAGAGAGATGGGTGCGAGAGCGTCGGTATTAAGC | |
| | GGGGGAGAATTAGATAAATGGGAAAAAATTCGGTTAAGGCCAGG | |
| | GGGAAAGAAACAATATAAACTAAAACATATAGTATGGGCAAGCA | |
| | GGGAGCTAGAACGATTCGCAGTTAATCCTGGCCTTTTAGAGACATC | |
| | AGAAGGCTGTAGACAAATACTGGGACAGCTACAACCATCCCTTCA | |
| | GACAGGATCAGAAGAACTTAGATCATTATATAATACAATAGCAGT | |
| | CCTCTATTGTGTGCATCAAAGGATAGATGTAAAAGACACCAAGGA | |
| | AGCCTTAGATAAGATAGAGGAAGAGCAAAACAAAAGTAAGAAAA | |
| | AGGCACAGCAAGCAGGTAGTGGCGAGGGCAGAGGAAGTCTTCTAA | |
| | CATGCGGTGACGTGGAGGAGAATCCCGGCCCTATGGTGCCCAAGA | |
| | AGAAGAGGAAAGTCTCCAACCTGCTGACTGTGCACCAAAACCTGC | |
| | CTGCCCTCCCTGTGGATGCCACCTCTGATGAAGTCAGGAAGAACCT | |
| | GATGGACATGTTCAGGGACAGGCAGGCCTTCTCTGAACACACCTG | |
| | GAAGATGCTCCTGTCTGTGTGCAGATCCTGGGCTGCCTGGTGCAAG | |
| | CTGAACAACAGGAAATGGTTCCCTGCTGAACCTGAGGATGTGAGG | |
| | GACTACCTCCTGTACCTGCAAGCCAGAGGCCTGGCTGTGAAGACC | |
| | ATCCAACAGCACCTGGGCCAGCTCAACATGCTGCACAGGAGATCT | |
| | GGCCTGCCTCGCCCTTCTGACTCCAATGCTGTGTCCCTGGTGATGA | |
| | GGAGAATCAGAAAGGAGAATGTGGATGCTGGGGAGAGAGCCAAG | |
| | CAGGCCCTGGCCTTTGAACGCACTGACTTTGACCAAGTCAGATCCC | |
| | TGATGGAGAACTCTGACAGATGCCAGGACATCAGGAACCTGGCCT | |
| | TCCTGGGCATTGCCTACAACACCCTGCTGCGCATTGCCGAAATTGC | |
| | CAGAATCAGAGTGAAGGACATCTCCCGCACCGATGGTGGGAGAAT | |
| | GCTGATCCACATTGGCAGGACCAAGACCCTGGTGTCCACAGCTGG | |
| | TGTGGAGAAGGCCCTGTCCCTGGGGGTTACCAAGCTGGTGGAGAG | |
| | ATGGATCTCTGTGTCTGGTGTGGCTGATGACCCCAACAACTACCTG | |
| | TTCTGCCGGGTCAGAAAGAATGGTGTGGCTGCCCCTTCTGCCACCT | |
| | CCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCTAATCAACCTCTGGATTACAAAATTTGTGAAAGATTGAC | |
| | TGGTATTCTTAACTATGTTGCTCCTTTTACGCTATGTGGATACGCTG | |
| | CTTTAATGCCTTTGTATCATGCTATTGCTTCCCGTATGGCTTTCATT | |
| | TTCTCCTCCTTGTATAAATCCTGGTTGCTGTCTCTTTATGAGGAGTT | |
| | GTGGCCCGTTGTCAGGCAACGTGGCGTGGTGTGCACTGTGTTTGCT | |
| | GACGCAACCCCCACTGGTTGGGGCATTGCCACCACCTGTCAGCTCC | |
| | TTTCCGGGACTTTCGCTTTCCCCCTCCCTATTGCCACGGCGGAACTC | |
| | ATCGCCGCCTGCCTTGCCCGCTGCTGGACAGGGGCTCGGCTGTTGG | |
| | GCACTGACAATTCCGTGGTGTTGTCGGGGAAATCATCGTCCTTTCC | |
| | TTGGCTGCTCGCCTGTGTTGCCACCTGGATTCTGCGCGGGACGTCC | |
| | TTCTGCTACGTCCCTTCGGCCCTCAATCCAGCGGACCTTCCTTCCCG | |
| | CGGCCTGCTGCCGGCTCTGCGGCCTCTTCCGCGTCTTCGCCTTCGCC | |
| | CTCAGACGAGTCGGATCTCCCTTTGGGCCGCCTCCCCGCCTGAGAT | |
| | CCTTTAAGACCAATGACTTACAAGGCAGCTGTAGATCTTAGCCACT | |
| | TTTTAAAAGAAAAGGGGGGACTGGAAGGGCTAATTCACTCCCAAC | |
| | GAAGACAAGATCTGCTTTTTGCTTGTACTGGGTCTCTCTGGTTAGA | |
| | CCAGATCTGAGCCTGGGAGCTCTCTGGCTAACTAGGGAACCCACT | |
| | GCTTAAGCCTCAATAAAGCTTGCCTTGAGTGCTTCAAGTAGTGTGT | |
| | GCCCGTCTGTTGTGTGACTCTGGTAACTAGAGATCCCTCAGACCCT | |
| | TTTAGTCAGTGTGGAAAATCTCTAGCAGTAGTAGTTCATGTCATCT | |
| | TATTATTCAGTATTTATAACTTGCAAAGAAATGAATATCAGAGAGT | |
| | GAGAGGCCCGGGTTAATTAAGGAAAGGGCTAGATCATTCTTGAAG | |
| | ACGAAAGGGCCTCGTGATACGCCTATTTTTATAGGTTAATGTCATG | |
| | ATAATAATGGTTTCTTAGACGTCAGGTGGCACTTTTCGGGGAAATG | |
| | TGCGCGGAACCCCTATTTGTTTATTTTTCTAAATACATTCAAATATG | |
| | TATCCGCTCATGAGACAATAACCCTGATAAATGCTTCAATAATATT | |
| | GAAAAAGGAAGAGTATGAGTATTCAACATTTCCGTGTCGCCCTTAT | |
| | TCCCTTTTTTGCGGCATTTTGCCTTCCTGTTTTTGCTCACCCAGAAA | |
| | CGCTGGTGAAAGTAAAAGATGCTGAAGATCAGTTGGGTGCACGAG | |
| | TGGGTTACATCGAACTGGATCTCAACAGCGGTAAGATCCTTGAGA | |
| | GTTTTCGCCCCGAAGAACGTTTTCCAATGATGAGCACTTTTAAAGT | |
| | TCTGCTATGTGGCGCGGTATTATCCCGTGTTGACGCCGGGCAAGAG | |
| | CAACTCGGTCGCCGCATACACTATTCTCAGAATGACTTGGTTGAGT | |
| | ACTCACCAGTCACAGAAAAGCATCTTACGGATGGCATGACAGTAA | |
| | GAGAATTATGCAGTGCTGCCATAACCATGAGTGATAACACTGCGG | |
| | CCAACTTACTTCTGACAACGATCGGAGGACCGAAGGAGCTAACCG | |
| | CTTTTTTGCACAACATGGGGGATCATGTAACTCGCCTTGATCGTTG | |
| | GGAACCGGAGCTGAATGAAGCCATACCAAACGACGAGCGTGACAC | |
| | CACGATGCCTGTAGCAATGGCAACAACGTTGCGCAAACTATTAAC | |
| | TGGCGAACTACTTACTCTAGCTTCCCGGCAACAATTAATAGACTGG | |
| | ATGGAGGCGGATAAAGTTGCAGGACCACTTCTGCGCTCGGCCCTTC | |
| | CGGCTGGCTGGTTTATTGCTGATAAATCTGGAGCCGGTGAGCGTGG | |
| | GTCTCGCGGTATCATTGCAGCACTGGGGCCAGATGGTAAGCCCTCC | |
| | CGTATCGTAGTTATCTACACGACGGGGAGTCAGGCAACTATGGAT | |
| | GAACGAAATAGACAGATCGCTGAGATAGGTGCCTCACTGATTAAG | |
| | CATTGGTAACTGTCAGACCAAGTTTACTCATATATACTTTAGATTG | |
| | ATTTAAAACTTCATTTTTAATTTAAAAGGATCTAGGTGAAGATCCT | |
| | TTTTGATAATCTCATGACCAAAATCCCTTAACGTGAGTTTTCGTTCC | |
| | ACTGAGCGTCAGACCCCGTAGAAAAGATCAAAGGATCTTCTTGAG | |
| | ATCCTTTTTTTCTGCGCGTAATCTGCTGCTTGCAAACAAAAAAACC | |
| | ACCGCTACCAGCGGTGGTTTGTTTGCCGGATCAAGAGCTACCAACT | |
| | CTTTTTCCGAAGGTAACTGGCTTCAGCAGAGCGCAGATACCAAATA | |
| | CTGTTCTTCTAGTGTAGCCGTAGTTAGGCCACCACTTCAAGAACTC | |
| | TGTAGCACCGCCTACATACCTCGCTCTGCTAATCCTGTTACCAGTG | |
| | GCTGCTGCCAGTGGCGATAAGTCGTGTCTTACCGGGTTGGACTCAA | |
| | GACGATAGTTACCGGATAAGGCGCAGCGGTCGGGCTGAACGGGGG | |
| | GTTCGTGCACACAGCCCAGCTTGGAGCGAACGACCTACACCGAAC | |
| | TGAGATACCTACAGCGTGAGCTATGAGAAAGCGCCACGCTTCCCG | |
| | AAGGGAGAAAGGCGGACAGGTATCCGGTAAGCGGCAGGGTCGGA | |
| | ACAGGAGAGCGCACGAGGGAGCTTCCAGGGGGAAACGCCTGGTAT | |
| | CTTTATAGTCCTGTCGGGTTTCGCCACCTCTGACTTGAGCGTCGATT | |
| | TTTGTGATGCTCGTCAGGGGGGCGGAGCCTATGGAAAAACGCCAG | |
| | CAACGCGGCCTTTTTACGGTTCCTGGCCTTTTGCTGGCCTTTTGCTC | |
| | ACATGTTCTTTCCTGCGTTATCCCCTGATTCTGTGGATAACCGTATT | |
| | ACCGCCTTTGAGTGAGCTGATACCGCTCGCCGCAGCCGAACGACC | |
| | GAGCGCAGCGAGTCAGTGAGCGAGGAAGCGGAAGAGCGCCCAAT | |
| | ACGCAAACCGCCTCTCCCCGCGCGTTGGCCGATTCATTAATGCAGC | |
| | AAGCTCATGGCTGACTAATTTTTTTTATTTATGCAGAGGCCGAGGC | |
| | CGCCTCGGCCTCTGAGCTATTCCAGAAGTAGTGAGGAGGCTTTTTT | |
| | GGAGGCCTAGGCTTTTGCAAAAAGCTCCCCGTGGCACGACAGGTT | |
| | TCCCGACTGGAAAGCGGGCAGTGAGCGCAACGCAATTAATGTGAG | |
| | TTAGCTCACTCATTAGGCACCCCAGGCTTTACACTTTATGCTTCCG | |
| | GCTCGTATGTTGTGTGGAATTGTGAGCGGATAACAATTTCACACAG | |
| | GAAACAGCTATGACATGATTACGAATTTCACAAATAAAGCATTTTT | |
| | TTCACTGCATTCTAGTTGTGGTTTGTCCAAACTCATCAATGTATCTT | |
| | ATCATGTCTGGATCAACTGGATAACTCAAGCTAACCAAAATCATCC | |
| | CAAACTTCCCACCCCATACCCTATTACCACTGCCAATTACCTGTGG | |
| | TTTCATTTACTCTAAACCTGTGATTCCTCTGAATTATTTTCATTTTA | |
| | AAGAAATTGTATTTGTTAAATATGTACTACAAACTTAGTAGT | |
| 142 | TGGAAGGGCTAATTCACTCCCAAAGAAGACAAGATATCCTTGATC | GAG-T2A- |
| | TGTGGATCTACCACACACAAGGCTACTTCCCTGATTAGCAGAACTA | EGFP |
| | CACACCAGGGCCAGGGGTCAGATATCCACTGACCTTTGGATGGTG | |
| | CTACAAGCTAGTACCAGTTGAGCCAGATAAGGTAGAAGAGGCCAA | |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGGTGAGTACGCCAAAAATTTTGACTAGCGG | |
| | AGGCTAGAAGGAGAGAGATGGGTGCGAGAGCGTCGGTATTAAGC | |
| | GGGGGAGAATTAGATAAATGGGAAAAAATTCGGTTAAGGCCAGG | |
| | GGGAAAGAAACAATATAAACTAAAACATATAGTATGGGCAAGCA | |
| | GGGAGCTAGAACGATTCGCAGTTAATCCTGGCCTTTTAGAGACATC | |
| | AGAAGGCTGTAGACAAATACTGGGACAGCTACAACCATCCCTTCA | |
| | GACAGGATCAGAAGAACTTAGATCATTATATAATACAATAGCAGT | |
| | CCTCTATTGTGTGCATCAAAGGATAGATGTAAAAGACACCAAGGA | |
| | AGCCTTAGATAAGATAGAGGAAGAGCAAAACAAAAGTAAGAAAA | |
| | AGGCACAGCAAGCAGGTAGTGGCGAGGGCAGAGGAAGTCTTCTAA | |
| | CATGCGGTGACGTGGAGGAGAATCCCGGCCCTATGGTGAGCAAGG | |
| | GCGAGGAGCTGTTCACCGGGGTGGTGCCCATCCTGGTCGAGCTGG | |
| | ACGGCGACGTAAACGGCCACAAGTTCAGCGTGTCCGGCGAGGGCG | |
| | AGGGCGATGCCACCTACGGCAAGCTGACCCTGAAGTTCATCTGCA | |
| | CCACCGGCAAGCTGCCCGTGCCCTGGCCCACCCTCGTGACCACCCT | |
| | GACCTACGGCGTGCAGTGCTTCAGCCGCTACCCCGACCACATGAA | |
| | GCAGCACGACTTCTTCAAGTCCGCCATGCCCGAAGGCTACGTCCAG | |
| | GAGCGCACCATCTTCTTCAAGGACGACGGCAACTACAAGACCCGC | |
| | GCCGAGGTGAAGTTCGAGGGCGACACCCTGGTGAACCGCATCGAG | |
| | CTGAAGGGCATCGACTTCAAGGAGGACGGCAACATCCTGGGGCAC | |
| | AAGCTGGAGTACAACTACAACAGCCACAACGTCTATATCATGGCC | |
| | GACAAGCAGAAGAACGGCATCAAGGTGAACTTCAAGATCCGCCAC | |
| | AACATCGAGGACGGCAGCGTGCAGCTCGCCGACCACTACCAGCAG | |
| | AACACCCCCATCGGCGACGGCCCCGTGCTGCTGCCCGACAACCAC | |
| | TACCTGAGCACCCAGTCCGCCCTGAGCAAAGACCCCAACGAGAAG | |
| | CGCGATCACATGGTCCTGCTGGAGTTCGTGACCGCCGCCGGGATCA | |
| | CTCTCGGCATGGACGAGCTGTACAAGTAAGGATCCTAATCAACCTC | |
| | TGGATTACAAAATTTGTGAAAGATTGACTGGTATTCTTAACTATGT | |
| | TGCTCCTTTTACGCTATGTGGATACGCTGCTTTAATGCCTTTGTATC | |
| | ATGCTATTGCTTCCCGTATGGCTTTCATTTTCTCCTCCTTGTATAAA | |
| | TCCTGGTTGCTGTCTCTTTATGAGGAGTTGTGGCCCGTTGTCAGGC | |
| | AACGTGGCGTGGTGTGCACTGTGTTTGCTGACGCAACCCCCACTGG | |
| | TTGGGGCATTGCCACCACCTGTCAGCTCCTTTCCGGGACTTTCGCTT | |
| | TCCCCCTCCCTATTGCCACGGCGGAACTCATCGCCGCCTGCCTTGC | |
| | CCGCTGCTGGACAGGGGCTCGGCTGTTGGGCACTGACAATTCCGTG | |
| | GTGTTGTCGGGGAAATCATCGTCCTTTCCTTGGCTGCTCGCCTGTGT | |
| | TGCCACCTGGATTCTGCGCGGGACGTCCTTCTGCTACGTCCCTTCG | |
| | GCCCTCAATCCAGCGGACCTTCCTTCCCGCGGCCTGCTGCCGGCTC | |
| | TGCGGCCTCTTCCGCGTCTTCGCCTTCGCCCTCAGACGAGTCGGAT | |
| | CTCCCTTTGGGCCGCCTCCCCGCCTGAGATCCTTTAAGACCAATGA | |
| | CTTACAAGGCAGCTGTAGATCTTAGCCACTTTTTAAAAGAAAAGG | |
| | GGGGACTGGAAGGGCTAATTCACTCCCAACGAAGACAAGATCTGC | |
| | TTTTTGCTTGTACTGGGTCTCTCTGGTTAGACCAGATCTGAGCCTGG | |
| | GAGCTCTCTGGCTAACTAGGGAACCCACTGCTTAAGCCTCAATAAA | |
| | GCTTGCCTTGAGTGCTTCAAGTAGTGTGTGCCCGTCTGTTGTGTGA | |
| | CTCTGGTAACTAGAGATCCCTCAGACCCTTTTAGTCAGTGTGGAAA | |
| | ATCTCTAGCAGTAGTAGTTCATGTCATCTTATTATTCAGTATTTATA | |
| | ACTTGCAAAGAAATGAATATCAGAGAGTGAGAGGCCCGGGTTAAT | |
| | TAAGGAAAGGGCTAGATCATTCTTGAAGACGAAAGGGCCTCGTGA | |
| | TACGCCTATTTTTATAGGTTAATGTCATGATAATAATGGTTTCTTAG | |
| | ACGTCAGGTGGCACTTTTCGGGGAAATGTGCGCGGAACCCCTATTT | |
| | GTTTATTTTTCTAAATACATTCAAATATGTATCCGCTCATGAGACA | |
| | ATAACCCTGATAAATGCTTCAATAATATTGAAAAAGGAAGAGTAT | |
| | GAGTATTCAACATTTCCGTGTCGCCCTTATTCCCTTTTTTGCGGCAT | |
| | TTTGCCTTCCTGTTTTTGCTCACCCAGAAACGCTGGTGAAAGTAAA | |
| | AGATGCTGAAGATCAGTTGGGTGCACGAGTGGGTTACATCGAACT | |
| | GGATCTCAACAGCGGTAAGATCCTTGAGAGTTTTCGCCCCGAAGA | |
| | ACGTTTTCCAATGATGAGCACTTTTAAAGTTCTGCTATGTGGCGCG | |
| | GTATTATCCCGTGTTGACGCCGGGCAAGAGCAACTCGGTCGCCGC | |
| | ATACACTATTCTCAGAATGACTTGGTTGAGTACTCACCAGTCACAG | |
| | AAAAGCATCTTACGGATGGCATGACAGTAAGAGAATTATGCAGTG | |
| | CTGCCATAACCATGAGTGATAACACTGCGGCCAACTTACTTCTGAC | |
| | AACGATCGGAGGACCGAAGGAGCTAACCGCTTTTTTGCACAACAT | |
| | GGGGGATCATGTAACTCGCCTTGATCGTTGGGAACCGGAGCTGAA | |
| | TGAAGCCATACCAAACGACGAGCGTGACACCACGATGCCTGTAGC | |
| | AATGGCAACAACGTTGCGCAAACTATTAACTGGCGAACTACTTACT | |
| | CTAGCTTCCCGGCAACAATTAATAGACTGGATGGAGGCGGATAAA | |
| | GTTGCAGGACCACTTCTGCGCTCGGCCCTTCCGGCTGGCTGGTTTA | |
| | TTGCTGATAAATCTGGAGCCGGTGAGCGTGGGTCTCGCGGTATCAT | |
| | TGCAGCACTGGGGCCAGATGGTAAGCCCTCCCGTATCGTAGTTATC | |
| | TACACGACGGGGAGTCAGGCAACTATGGATGAACGAAATAGACAG | |
| | ATCGCTGAGATAGGTGCCTCACTGATTAAGCATTGGTAACTGTCAG | |
| | ACCAAGTTTACTCATATATACTTTAGATTGATTTAAAACTTCATTTT | |
| | TAATTTAAAAGGATCTAGGTGAAGATCCTTTTTGATAATCTCATGA | |
| | CCAAAATCCCTTAACGTGAGTTTTCGTTCCACTGAGCGTCAGACCC | |
| | CGTAGAAAAGATCAAAGGATCTTCTTGAGATCCTTTTTTTCTGCGC | |
| | GTAATCTGCTGCTTGCAAACAAAAAAACCACCGCTACCAGCGGTG | |
| | GTTTGTTTGCCGGATCAAGAGCTACCAACTCTTTTTCCGAAGGTAA | |
| | CTGGCTTCAGCAGAGCGCAGATACCAAATACTGTTCTTCTAGTGTA | |
| | GCCGTAGTTAGGCCACCACTTCAAGAACTCTGTAGCACCGCCTACA | |
| | TACCTCGCTCTGCTAATCCTGTTACCAGTGGCTGCTGCCAGTGGCG | |
| | ATAAGTCGTGTCTTACCGGGTTGGACTCAAGACGATAGTTACCGGA | |
| | TAAGGCGCAGCGGTCGGGCTGAACGGGGGGTTCGTGCACACAGCC | |
| | CAGCTTGGAGCGAACGACCTACACCGAACTGAGATACCTACAGCG | |
| | TGAGCTATGAGAAAGCGCCACGCTTCCCGAAGGGAGAAAGGCGGA | |
| | CAGGTATCCGGTAAGCGGCAGGGTCGGAACAGGAGAGCGCACGA | |
| | GGGAGCTTCCAGGGGGAAACGCCTGGTATCTTTATAGTCCTGTCGG | |
| | GTTTCGCCACCTCTGACTTGAGCGTCGATTTTTGTGATGCTCGTCAG | |
| | GGGGGCGGAGCCTATGGAAAAACGCCAGCAACGCGGCCTTTTTAC | |
| | GGTTCCTGGCCTTTTGCTGGCCTTTTGCTCACATGTTCTTTCCTGCG | |
| | TTATCCCCTGATTCTGTGGATAACCGTATTACCGCCTTTGAGTGAG | |
| | CTGATACCGCTCGCCGCAGCCGAACGACCGAGCGCAGCGAGTCAG | |
| | TGAGCGAGGAAGCGGAAGAGCGCCCAATACGCAAACCGCCTCTCC | |
| | CCGCGCGTTGGCCGATTCATTAATGCAGCAAGCTCATGGCTGACTA | |
| | ATTTTTTTTATTTATGCAGAGGCCGAGGCCGCCTCGGCCTCTGAGC | |
| | TATTCCAGAAGTAGTGAGGAGGCTTTTTTGGAGGCCTAGGCTTTTG | |
| | CAAAAAGCTCCCCGTGGCACGACAGGTTTCCCGACTGGAAAGCGG | |
| | GCAGTGAGCGCAACGCAATTAATGTGAGTTAGCTCACTCATTAGG | |
| | CACCCCAGGCTTTACACTTTATGCTTCCGGCTCGTATGTTGTGTGG | |
| | AATTGTGAGCGGATAACAATTTCACACAGGAAACAGCTATGACAT | |
| | GATTACGAATTTCACAAATAAAGCATTTTTTTCACTGCATTCTAGTT | |
| | GTGGTTTGTCCAAACTCATCAATGTATCTTATCATGTCTGGATCAA | |
| | CTGGATAACTCAAGCTAACCAAAATCATCCCAAACTTCCCACCCCA | |
| | TACCCTATTACCACTGCCAATTACCTGTGGTTTCATTTACTCTAAAC | |
| | CTGTGATTCCTCTGAATTATTTTCATTTTAAAGAAATTGTATTTGTT | |
| | AAATATGTACTACAAACTTAGTAGT | |
| 143 | TGGAAGGGCTAATTCACTCCCAAAGAAGACAAGATATCCTTGATC | N-term |
| | TGTGGATCTACCACACACAAGGCTACTTCCCTGATTAGCAGAACTA | mutated MSD |
| | CACACCAGGGCCAGGGGTCAGATATCCACTGACCTTTGGATGGTG | GAG-T2A- |
| | CTACAAGCTAGTACCAGTTGAGCCAGATAAGGTAGAAGAGGCCAA | EGFP |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGGTGAGTACGCCAAAAATTTTGACTAGCGG | |
| | AGGCTAGAAGGAGAGAGATGGGTGCGAGAGCGTCGGTATTAAGC | |
| | GGGGGAGAATTAGATAAATGGGAAAAAATTCGGTTAAGGCCAGG | |
| | GGGAAAGAAACAATATAAACTAAAACATATAGTATGGGCAAGCA | |
| | GGGAGCTAGAACGATTCGCAGTTAATCCTGGCCTTTTAGAGACATC | |
| | AGAAGGCTGTAGACAAATACTGGGACAGCTACAACCATCCCTTCA | |
| | GACAGGATCAGAAGAACTTAGATCATTATATAATACAATAGCAGT | |
| | CCTCTATTGTGTGCATCAAAGGATAGATGTAAAAGACACCAAGGA | |
| | AGCCTTAGATAAGATAGAGGAAGAGCAAAACAAAAGTAAGAAAA | |
| | AGGCACAGCAAGCAGGTAGTGGCGAGGGCAGAGGAAGTCTTCTAA | |
| | CATGCGGTGACGTGGAGGAGAATCCCGGCCCTATGGTGAGCAAGG | |
| | GCGAGGAGCTGTTCACCGGGGTGGTGCCCATCCTGGTCGAGCTGG | |
| | ACGGCGACGTAAACGGCCACAAGTTCAGCGTGTCCGGCGAGGGCG | |
| | AGGGCGATGCCACCTACGGCAAGCTGACCCTGAAGTTCATCTGCA | |
| | CCACCGGCAAGCTGCCCGTGCCCTGGCCCACCCTCGTGACCACCCT | |
| | GACCTACGGCGTGCAGTGCTTCAGCCGCTACCCCGACCACATGAA | |
| | GCAGCACGACTTCTTCAAGTCCGCCATGCCCGAAGGCTACGTCCAG | |
| | GAGCGCACCATCTTCTTCAAGGACGACGGCAACTACAAGACCCGC | |
| | GCCGAGGTGAAGTTCGAGGGCGACACCCTGGTGAACCGCATCGAG | |
| | CTGAAGGGCATCGACTTCAAGGAGGACGGCAACATCCTGGGGCAC | |
| | AAGCTGGAGTACAACTACAACAGCCACAACGTCTATATCATGGCC | |
| | GACAAGCAGAAGAACGGCATCAAGGTGAACTTCAAGATCCGCCAC | |
| | AACATCGAGGACGGCAGCGTGCAGCTCGCCGACCACTACCAGCAG | |
| | AACACCCCCATCGGCGACGGCCCCGTGCTGCTGCCCGACAACCAC | |
| | TACCTGAGCACCCAGTCCGCCCTGAGCAAAGACCCCAACGAGAAG | |
| | CGCGATCACATGGTCCTGCTGGAGTTCGTGACCGCCGCCGGGATCA | |
| | CTCTCGGCATGGACGAGCTGTACAAGTAAGGATCCTAATCAACCTC | |
| | TGGATTACAAAATTTGTGAAAGATTGACTGGTATTCTTAACTATGT | |
| | TGCTCCTTTTACGCTATGTGGATACGCTGCTTTAATGCCTTTGTATC | |
| | ATGCTATTGCTTCCCGTATGGCTTTCATTTTCTCCTCCTTGTATAAA | |
| | TCCTGGTTGCTGTCTCTTTATGAGGAGTTGTGGCCCGTTGTCAGGC | |
| | AACGTGGCGTGGTGTGCACTGTGTTTGCTGACGCAACCCCCACTGG | |
| | TTGGGGCATTGCCACCACCTGTCAGCTCCTTTCCGGGACTTTCGCTT | |
| | TCCCCCTCCCTATTGCCACGGCGGAACTCATCGCCGCCTGCCTTGC | |
| | CCGCTGCTGGACAGGGGCTCGGCTGTTGGGCACTGACAATTCCGTG | |
| | GTGTTGTCGGGGAAATCATCGTCCTTTCCTTGGCTGCTCGCCTGTGT | |
| | TGCCACCTGGATTCTGCGCGGGACGTCCTTCTGCTACGTCCCTTCG | |
| | GCCCTCAATCCAGCGGACCTTCCTTCCCGCGGCCTGCTGCCGGCTC | |
| | TGCGGCCTCTTCCGCGTCTTCGCCTTCGCCCTCAGACGAGTCGGAT | |
| | CTCCCTTTGGGCCGCCTCCCCGCCTGAGATCCTTTAAGACCAATGA | |
| | CTTACAAGGCAGCTGTAGATCTTAGCCACTTTTTAAAAGAAAAGG | |
| | GGGGACTGGAAGGGCTAATTCACTCCCAACGAAGACAAGATCTGC | |
| | TTTTTGCTTGTACTGGGTCTCTCTGGTTAGACCAGATCTGAGCCTGG | |
| | GAGCTCTCTGGCTAACTAGGGAACCCACTGCTTAAGCCTCAATAAA | |
| | GCTTGCCTTGAGTGCTTCAAGTAGTGTGTGCCCGTCTGTTGTGTGA | |
| | CTCTGGTAACTAGAGATCCCTCAGACCCTTTTAGTCAGTGTGGAAA | |
| | ATCTCTAGCAGTAGTAGTTCATGTCATCTTATTATTCAGTATTTATA | |
| | ACTTGCAAAGAAATGAATATCAGAGAGTGAGAGGCCCGGGTTAAT | |
| | TAAGGAAAGGGCTAGATCATTCTTGAAGACGAAAGGGCCTCGTGA | |
| | TACGCCTATTTTTATAGGTTAATGTCATGATAATAATGGTTTCTTAG | |
| | ACGTCAGGTGGCACTTTTCGGGGAAATGTGCGCGGAACCCCTATTT | |
| | GTTTATTTTTCTAAATACATTCAAATATGTATCCGCTCATGAGACA | |
| | ATAACCCTGATAAATGCTTCAATAATATTGAAAAAGGAAGAGTAT | |
| | GAGTATTCAACATTTCCGTGTCGCCCTTATTCCCTTTTTTGCGGCAT | |
| | TTTGCCTTCCTGTTTTTGCTCACCCAGAAACGCTGGTGAAAGTAAA | |
| | AGATGCTGAAGATCAGTTGGGTGCACGAGTGGGTTACATCGAACT | |
| | GGATCTCAACAGCGGTAAGATCCTTGAGAGTTTTCGCCCCGAAGA | |
| | ACGTTTTCCAATGATGAGCACTTTTAAAGTTCTGCTATGTGGCGCG | |
| | GTATTATCCCGTGTTGACGCCGGGCAAGAGCAACTCGGTCGCCGC | |
| | ATACACTATTCTCAGAATGACTTGGTTGAGTACTCACCAGTCACAG | |
| | AAAAGCATCTTACGGATGGCATGACAGTAAGAGAATTATGCAGTG | |
| | CTGCCATAACCATGAGTGATAACACTGCGGCCAACTTACTTCTGAC | |
| | AACGATCGGAGGACCGAAGGAGCTAACCGCTTTTTTGCACAACAT | |
| | GGGGGATCATGTAACTCGCCTTGATCGTTGGGAACCGGAGCTGAA | |
| | TGAAGCCATACCAAACGACGAGCGTGACACCACGATGCCTGTAGC | |
| | AATGGCAACAACGTTGCGCAAACTATTAACTGGCGAACTACTTACT | |
| | CTAGCTTCCCGGCAACAATTAATAGACTGGATGGAGGCGGATAAA | |
| | GTTGCAGGACCACTTCTGCGCTCGGCCCTTCCGGCTGGCTGGTTTA | |
| | TTGCTGATAAATCTGGAGCCGGTGAGCGTGGGTCTCGCGGTATCAT | |
| | TGCAGCACTGGGGCCAGATGGTAAGCCCTCCCGTATCGTAGTTATC | |
| | TACACGACGGGGAGTCAGGCAACTATGGATGAACGAAATAGACAG | |
| | ATCGCTGAGATAGGTGCCTCACTGATTAAGCATTGGTAACTGTCAG | |
| | ACCAAGTTTACTCATATATACTTTAGATTGATTTAAAACTTCATTTT | |
| | TAATTTAAAAGGATCTAGGTGAAGATCCTTTTTGATAATCTCATGA | |
| | CCAAAATCCCTTAACGTGAGTTTTCGTTCCACTGAGCGTCAGACCC | |
| | CGTAGAAAAGATCAAAGGATCTTCTTGAGATCCTTTTTTTCTGCGC | |
| | GTAATCTGCTGCTTGCAAACAAAAAAACCACCGCTACCAGCGGTG | |
| | GTTTGTTTGCCGGATCAAGAGCTACCAACTCTTTTTCCGAAGGTAA | |
| | CTGGCTTCAGCAGAGCGCAGATACCAAATACTGTTCTTCTAGTGTA | |
| | GCCGTAGTTAGGCCACCACTTCAAGAACTCTGTAGCACCGCCTACA | |
| | TACCTCGCTCTGCTAATCCTGTTACCAGTGGCTGCTGCCAGTGGCG | |
| | ATAAGTCGTGTCTTACCGGGTTGGACTCAAGACGATAGTTACCGGA | |
| | TAAGGCGCAGCGGTCGGGCTGAACGGGGGGTTCGTGCACACAGCC | |
| | CAGCTTGGAGCGAACGACCTACACCGAACTGAGATACCTACAGCG | |
| | TGAGCTATGAGAAAGCGCCACGCTTCCCGAAGGGAGAAAGGCGGA | |
| | CAGGTATCCGGTAAGCGGCAGGGTCGGAACAGGAGAGCGCACGA | |
| | GGGAGCTTCCAGGGGGAAACGCCTGGTATCTTTATAGTCCTGTCGG | |
| | GTTTCGCCACCTCTGACTTGAGCGTCGATTTTTGTGATGCTCGTCAG | |
| | GGGGGCGGAGCCTATGGAAAAACGCCAGCAACGCGGCCTTTTTAC | |
| | GGTTCCTGGCCTTTTGCTGGCCTTTTGCTCACATGTTCTTTCCTGCG | |
| | TTATCCCCTGATTCTGTGGATAACCGTATTACCGCCTTTGAGTGAG | |
| | CTGATACCGCTCGCCGCAGCCGAACGACCGAGCGCAGCGAGTCAG | |
| | TGAGCGAGGAAGCGGAAGAGCGCCCAATACGCAAACCGCCTCTCC | |
| | CCGCGCGTTGGCCGATTCATTAATGCAGCAAGCTCATGGCTGACTA | |
| | ATTTTTTTTATTTATGCAGAGGCCGAGGCCGCCTCGGCCTCTGAGC | |
| | TATTCCAGAAGTAGTGAGGAGGCTTTTTTGGAGGCCTAGGCTTTTG | |
| | CAAAAAGCTCCCCGTGGCACGACAGGTTTCCCGACTGGAAAGCGG | |
| | GCAGTGAGCGCAACGCAATTAATGTGAGTTAGCTCACTCATTAGG | |
| | CACCCCAGGCTTTACACTTTATGCTTCCGGCTCGTATGTTGTGTGG | |
| | AATTGTGAGCGGATAACAATTTCACACAGGAAACAGCTATGACAT | |
| | GATTACGAATTTCACAAATAAAGCATTTTTTTCACTGCATTCTAGTT | |
| | GTGGTTTGTCCAAACTCATCAATGTATCTTATCATGTCTGGATCAA | |
| | CTGGATAACTCAAGCTAACCAAAATCATCCCAAACTTCCCACCCCA | |
| | TACCCTATTACCACTGCCAATTACCTGTGGTTTCATTTACTCTAAAC | |
| | CTGTGATTCCTCTGAATTATTTTCATTTTAAAGAAATTGTATTTGTT | |
| | AAATATGTACTACAAACTTAGTAGT | |
| 144 | MVVILDKRCYCNLLILILMISECSVGILHYEKLSKIGLVKGVTRKYKIK | NiV F for VLP |
| | SNPLTKDIVIKMIPNVSNMSQCTGSVMENYKTRLNGILTPIKGALEIYK | |
| | NNTHDLVGDVRLAGVIMAGVAIGIATAAQITAGVALYEAMKNADNI | |
| | NKLKSSIESTNEAVVKLQETAEKTVYVLTALQDYINTNLVPTIDKISCK | |
| | QTELSLDLALSKYLSDLLFVFGPNLQDPVSNSMTIQAISQAFGGNYETL | |
| | LRTLGYATEDFDDLLESDSITGQIIYVDLSSYYIIVRVYFPILTEIQQAYI | |
| | QELLPVSFNNDNSEWISIVPNFILVRNTLISNIEIGFCLITKRSVICNQDY | |
| | ATPMTNNMRECLTGSTEKCPRELVVSSHVPRFALSNGVLFANCISVTC | |
| | QCQTTGRAISQSGEQTLLMIDNTTCPTAVLGNVIISLGKYLGSVNYNSE | |
| | GIAIGPPVFTDKVDISSQISSMNQSLQQSKDYIKEAQRLLDTVNPSLISM | |
| | LSMIILYVLSIASLCIGLITFISFIIVEKKRNT | |
| 145 | VVILDKRCYCNLLILILMISECSVGILHYEKLSKIGLVKGVTRKYKIKSN | NiV F for VLP |
| | PLTKDIVIKMIPNVSNMSQCTGSVMENYKTRLNGILTPIKGALEIYKNN | without N term |
| | THDLVGDVRLAGVIMAGVAIGIATAAQITAGVALYEAMKNADNINKL | Methionine |
| | KSSIESTNEAVVKLQETAEKTVYVLTALQDYINTNLVPTIDKISCKQTE | |
| | LSLDLALSKYLSDLLFVFGPNLQDPVSNSMTIQAISQAFGGNYETLLRT | |
| | LGYATEDFDDLLESDSITGQIIYVDLSSYYIIVRVYFPILTEIQQAYIQEL | |
| | LPVSFNNDNSEWISIVPNFILVRNTLISNIEIGFCLITKRSVICNQDYATP | |
| | MTNNMRECLTGSTEKCPRELVVSSHVPRFALSNGVLFANCISVTCQC | |
| | QTTGRAISQSGEQTLLMIDNTTCPTAVLGNVIISLGKYLGSVNYNSEGI | |
| | AIGPPVFTDKVDISSQISSMNQSLQQSKDYIKEAQRLLDTVNPSLISMLS | |
| | MIILYVLSIASLCIGLITFISFIIVEKKRNT | |
| 146 | MKKINEGLLDSKILSAFNTVIALLGSIVIIVMNIMIIQNYTRSTDNQAVI | Niv G for VLP |
| | KDALQGIQQQIKGLADKIGTEIGPKVSLIDTSSTITIPANIGLLGSKISQS | |
| | TASINENVNEKCKFTLPPLKIHECNISCPNPLPFREYRPQTEGVSNLVGL | |
| | PNNICLQKTSNQILKPKLISYTLPVVGQSGTCITDPLLAMDEGYFAYSH | |
| | LERIGSCSRGVSKQRIIGVGEVLDRGDEVPSLFMTNVWTPPNPNTVYH | |
| | CSAVYNNEFYYVLCAVSTVGDPILNSTYWSGSLMMTRLAVKPKSNG | |
| | GGYNQHQLALRSIEKGRYDKVMPYGPSGIKQGDTLYFPAVGFLVRTE | |
| | FKYNDSNCPITKCQYSKPENCRLSMGIRPNSHYILRSGLLKYNLSDGE | |
| | NPKVVFIEISDQRLSIGSPSKIYDSLGQPVFYQASFSWDTMIKFGDVLT | |
| | VNPLVVNWRNNTVISRPGQSQCPRFNTCPEICWEGVYNDAFLIDRINW | |
| | ISAGVFLDSNQTAENPVFTVFKDNEILYRAQLASEDTNAQKTITNCFLL | |
| | KNKIWCISLVEIYDTGDNVIRPKLFAVKIPEQCT | |
| 147 | KKINEGLLDSKILSAFNTVIALLGSIVIIVMNIMIIQNYTRSTDNQAVIKD | Niv G for VLP |
| | ALQGIQQQIKGLADKIGTEIGPKVSLIDTSSTITIPANIGLLGSKISQSTAS | without N term |
| | INENVNEKCKFTLPPLKIHECNISCPNPLPFREYRPQTEGVSNLVGLPN | Methionine |
| | NICLQKTSNQILKPKLISYTLPVVGQSGTCITDPLLAMDEGYFAYSHLE | |
| | RIGSCSRGVSKQRIIGVGEVLDRGDEVPSLFMTNVWTPPNPNTVYHCS | |
| | AVYNNEFYYVLCAVSTVGDPILNSTYWSGSLMMTRLAVKPKSNGGG | |
| | YNQHQLALRSIEKGRYDKVMPYGPSGIKQGDTLYFPAVGFLVRTEFK | |
| | YNDSNCPITKCQYSKPENCRLSMGIRPNSHYILRSGLLKYNLSDGENP | |
| | KVVFIEISDQRLSIGSPSKIYDSLGQPVFYQASFSWDTMIKFGDVLTVN | |
| | PLVVNWRNNTVISRPGQSQCPRFNTCPEICWEGVYNDAFLIDRINWIS | |
| | AGVFLDSNQTAENPVFTVFKDNEILYRAQLASEDTNAQKTITNCFLLK | |
| | NKIWCISLVEIYDTGDNVIRPKLFAVKIPEQCT | |
| 148 | AGCTTGGCCCATTGCATACGTTGTATCCATATCATAATATGTACAT | MS2_{cp}-EGFP |
| | TTATATTGGCTCATGTCCAACATTACCGCCATGTTGACATTGATTAT | mRNA; |
| | TGACTAGTTATTAATAGTAATCAATTACGGGGTCATTAGTTCATAG | HIV MA (N- |
| | CCCATATATGGAGTTCCGCGTTACATAACTTACGGTAAATGGCCCG | term) |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCATGGTGAGCAAGGGC | |
| | GAGGAGCTGTTCACCGGGGTGGTGCCCATCCTGGTCGAGCTGGAC | |
| | GGCGACGTAAACGGCCACAAGTTCAGCGTGTCCGGCGAGGGCGAG | |
| | GGCGATGCCACCTACGGCAAGCTGACCCTGAAGTTCATCTGCACC | |
| | ACCGGCAAGCTGCCCGTGCCCTGGCCCACCCTCGTGACCACCCTGA | |
| | CCTACGGCGTGCAGTGCTTCAGCCGCTACCCCGACCACATGAAGC | |
| | AGCACGACTTCTTCAAGTCCGCCATGCCCGAAGGCTACGTCCAGG | |
| | AGCGCACCATCTTCTTCAAGGACGACGGCAACTACAAGACCCGCG | |
| | CCGAGGTGAAGTTCGAGGGCGACACCCTGGTGAACCGCATCGAGC | |
| | TGAAGGGCATCGACTTCAAGGAGGACGGCAACATCCTGGGGCACA | |
| | AGCTGGAGTACAACTACAACAGCCACAACGTCTATATCATGGCCG | |
| | ACAAGCAGAAGAACGGCATCAAGGTGAACTTCAAGATCCGCCACA | |
| | ACATCGAGGACGGCAGCGTGCAGCTCGCCGACCACTACCAGCAGA | |
| | ACACCCCCATCGGCGACGGCCCCGTGCTGCTGCCCGACAACCACT | |
| | ACCTGAGCACCCAGTCCGCCCTGAGCAAAGACCCCAACGAGAAGC | |
| | GCGATCACATGGTCCTGCTGGAGTTCGTGACCGCCGCCGGGATCAC | |
| | TCTCGGCATGGACGAGCTGTACAAGTAAGGATCCAACCTACAAAC | |
| | GGGTGGAGGATCACCCCACCCGACACTTCACAATCAAGGGGTACA | |
| | ATACACAAGGGTGGAGGAACACCCCACCCTCCAGACACATTACAC | |
| | AGAAATCCAATCAAACAGAAGCACCATCAGGGCTTCTGCTACCAA | |
| | ATTTATCTCAAAAAACTACAACAAGGAATCACCATCAGGGATTCC | |
| | CTGTGCAATATACGTCAAACGAGGGCCACGACGGGAGGACGATCA | |
| | CGCCTCCCGAATATCGGCATGTCTGGCTTTCGAATTCAGTGCGTGG | |
| | AGCATCAGCCCACGCAGCCAATCAGAGTCGAATACAAGTCGACTT | |
| | TCGCGAAGAGCATCAGCCTTCGCGCCATTCTTACACAAACCACACT | |
| | CTCCCCTACAGGAACAGCATCAGCGTTCCTGCCCAGTACCCAACTC | |
| | AAGAAAATTTATGTCCCCATGCAGCATCAGCGCATGGGCCCCAAG | |
| | AATACATCCCCAACAAAATCACATCCGAGCACCAACAGGGCTCGG | |
| | AGTGTTGTTTCTTGTCCAACTGGACAAACCCTCCATGGACCATCAG | |
| | GCCATGGACTCTCACCAACAAGACAAAAACTACTCTTCTCGAAGC | |
| | AGCATCAGCGCTTCGAAACACTCGACCTCGAGGGCCCAGATCTAA | |
| | TTCACCCCACCAGTGCAGGCTGCCTATCAGAAAGTGGTGGCTGGTG | |
| | TGGCTAATGCCCTGGCCCACAAGTATCACTAAGCTCGCTTTCTTGC | |
| | TGTCCAATTTCTATTAAAGGTTCCTTTGTTCCCTAAGTCCAACTACT | |
| | AAACTGGGGGATATTATGAAGGGCCTTGAGCATCTGGATTCTGCCT | |
| | AATAAAAAACATTTATTTTCATTGCAATGATGTATTTAAATTATTTC | |
| | TGAATATTTTACTAAAAAGGGAATGTGGGAGGTCAGTGCATTTAA | |
| | AACATAAAGAAATGAAGAGCTAGTTCAAACCTTGGGAAAATACAC | |
| | TATATCTTAAACTCCATGAAAGAAGGTGAGGCTGCAAACAGCTAA | |
| | TGCACATTGGCAACAGCCCCTGATGCCTATGCCTTATTCATCCCTC | |
| | AGAAAAGGATTCAAGTAGAGGCTTGATTTGGAGGTTAAAGTTTTG | |
| | CTATGCTGTATTTTACATTACTTATTGTTTTAGCTGTCCTCATGAAT | |
| | GTCTTTTCACTACCCATTTGCTTATCCTGCATCTCTCAGCCTTGACT | |
| | CCACTCAGTTCTCTTGCTTAGAGATACCACCTTTCCCCTGAAGTGTT | |
| | CCTTCCATGTTTTACGGCGAGATGGTTTCTCCTCGCCTGGCCACTCA | |
| | GCCTTAGTTGTCTCTGTTGTCTTATAGAGGTCTACTTGAAGAAGGA | |
| | AAAACAGGGGGCATGGTTTGACTGTCCTGTGAGCCCTTCTTCCCTG | |
| | CCTCCCCCACTCACAGTGACCCGGAATCCCTCGACATGGCAGTCTA | |
| | GATCATTCTTGAAGACGAAAGGGCCTCGTGATACGCCTATTTTTAT | |
| | AGGTTAATGTCATGATAATAATGGTTTCTTAGACGTCAGGTGGCAC | |
| | TTTTCGGGGAAATGTGCGCGGAACCCCTATTTGTTTATTTTTCTAAA | |
| | TACATTCAAATATGTATCCGCTCATGAGACAATAACCCTGATAAAT | |
| | GCTTCAATAATATTGAAAAAGGAAGAGTATGAGTATTCAACATTTC | |
| | CGTGTCGCCCTTATTCCCTTTTTTGCGGCATTTTGCCTTCCTGTTTTT | |
| | GCTCACCCAGAAACGCTGGTGAAAGTAAAAGATGCTGAAGATCAG | |
| | TTGGGTGCACGAGTGGGTTACATCGAACTGGATCTCAACAGCGGT | |
| | AAGATCCTTGAGAGTTTTCGCCCCGAAGAACGTTTTCCAATGATGA | |
| | GCACTTTTAAAGTTCTGCTATGTGGCGCGGTATTATCCCGTATTGA | |
| | CGCCGGGCAAGAGCAACTCGGTCGCCGCATACACTATTCTCAGAA | |
| | TGACTTGGTTGAGTACTCACCAGTCACAGAAAAGCATCTTACGGAT | |
| | GGCATGACAGTAAGAGAATTATGCAGTGCTGCCATAACCATGAGT | |
| | GATAACACTGCGGCCAACTTACTTCTGACAACGATCGGAGGACCG | |
| | AAGGAGCTAACCGCTTTTTTGCACAACATGGGGGATCATGTAACTC | |
| | GCCTTGATCGTTGGGAACCGGAGCTGAATGAAGCCATACCAAACG | |
| | ACGAGCGTGACACCACGATGCCTGTAGCAATGGCAACAACGTTGC | |
| | GCAAACTATTAACTGGCGAACTACTTACTCTAGCTTCCCGGCAACA | |
| | ATTAATAGACTGGATGGAGGCGGATAAAGTTGCAGGACCACTTCT | |
| | GCGCTCGGCCCTTCCGGCTGGCTGGTTTATTGCTGATAAATCTGGA | |
| | GCCGGTGAGCGTGGGTCTCGCGGTATCATTGCAGCACTGGGGCCA | |
| | GATGGTAAGCCCTCCCGTATCGTAGTTATCTACACGACGGGGAGTC | |
| | AGGCAACTATGGATGAACGAAATAGACAGATCGCTGAGATAGGTG | |
| | CCTCACTGATTAAGCATTGGTAACTGTCAGACCAAGTTTACTCATA | |
| | TATACTTTAGATTGATTTAAAACTTCATTTTTAATTTAAAAGGATCT | |
| | AGGTGAAGATCCTTTTTGATAATCTCATGACCAAAATCCCTTAACG | |
| | TGAGTTTTCGTTCCACTGAGCGTCAGACCCCGTAGAAAAGATCAAA | |
| | GGATCTTCTTGAGATCCTTTTTTTCTGCGCGTAATCTGCTGCTTGCA | |
| | AACAAAAAAACCACCGCTACCAGCGGTGGTTTGTTTGCCGGATCA | |
| | AGAGCTACCAACTCTTTTTCCGAAGGTAACTGGCTTCAGCAGAGCG | |
| | CAGATACCAAATACTGTTCTTCTAGTGTAGCCGTAGTTAGGCCACC | |
| | ACTTCAAGAACTCTGTAGCACCGCCTACATACCTCGCTCTGCTAAT | |
| | CCTGTTACCAGTGGCTGCTGCCAGTGGCGATAAGTCGTGTCTTACC | |
| | GGGTTGGACTCAAGACGATAGTTACCGGATAAGGCGCAGCGGTCG | |
| | GGCTGAACGGGGGGTTCGTGCACACAGCCCAGCTTGGAGCGAACG | |
| | ACCTACACCGAACTGAGATACCTACAGCGTGAGCTATGAGAAAGC | |
| | GCCACGCTTCCCGAAGGGAGAAAGGCGGACAGGTATCCGGTAAGC | |
| | GGCAGGGTCGGAACAGGAGAGCGCACGAGGGAGCTTCCAGGGGG | |
| | AAACGCCTGGTATCTTTATAGTCCTGTCGGGTTTCGCCACCTCTGA | |
| | CTTGAGCGTCGATTTTTGTGATGCTCGTCAGGGGGGCGGAGCCTAT | |
| | GGAAAAACGCCAGCAACGGATGCGCCGCGTGCGGCTGCTGGAGAT | |
| | GGCGGACGCGATGGATATGTTCTGCCAAGGGTTGGTTTGCGCATTC | |
| | ACAGTTCTCCGCAAGAATTGATTGGCTCCAATTCTTGGAGTGGTGA | |
| | ATCCGTTAGCGAGGTGCCGCCGGCTTCCATTCAGGTCGAGGTGGCC | |
| | CGGCTCCATGCACCGCGACGCAACGCGGGGAGGCAGACAAGGTAT | |
| | AGGGCGGCGCCTACAATCCATGCCAACCCGTTCCATGTGCTCGCCG | |
| | AGGCGGCATAAATCCCCGTGACGATCAGCGGTCCAATGATCGAAG | |
| | TTAGGCTGGTAAGAGCCGCGAGCGATCCTTGAAGCTGTCCCTGATG | |
| | GTCGTCATCTACCTGCCTGGACAGCATGGCCTGCAACGCGGGCATC | |
| | CCGATGCCGCCGGAAGCGAGAAGAATCATAATGGGGAAGGCCATC | |
| | CAGCCTCGCGTCGGGGAGCTTTTTGCAAAAGCCTAGGCCTCCAAA | |
| | AAAGCCTCCTCACTACTTCTGGAATAGCTCAGAGGCCGAGGCGGC | |
| | CTCGGCCTCTGCATAAATAAAAAAAATTAGTCAGCCATG | |
| 149 | AGCTTGGCCCATTGCATACGTTGTATCCATATCATAATATGTACAT | MA-EGFP; |
| | TTATATTGGCTCATGTCCAACATTACCGCCATGTTGACATTGATTAT | HIV MA (N- |
| | TGACTAGTTATTAATAGTAATCAATTACGGGGTCATTAGTTCATAG | term) |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCCGC | |
| | GCCTCCGTGCTGTCCGGCGGCGAGCTGGACAAGTGGGAGAAGATC | |
| | CGCCTGCGCCCCGGCGGCAAGAAGCAGTACAAGCTGAAGCACATC | |
| | GTGTGGGCCTCCCGCGAGCTGGAGCGCTTCGCCGTGAACCCCGGC | |
| | CTGCTGGAAACCTCCGAGGGCTGCCGCCAGATCCTGGGCCAGCTG | |
| | CAGCCCTCCCTGCAAACCGGCTCCGAGGAGCTGCGCTCCCTGTACA | |
| | ACACCATCGCCGTGCTGTACTGCGTGCACCAGCGCATCGACGTGA | |
| | AGGACACCAAGGAGGCCCTGGACAAGATCGAGGAGGAGCAGAAC | |
| | AAGTCCAAGAAGAAGGCCCAGCAGGCCGCCGCCGACACCGGCAA | |
| | CAACTCCCAGGTGTCCCAGAACTACCCCATCGTGCAGATGGTGAG | |
| | CAAGGGCGAGGAGCTGTTCACCGGGGTGGTGCCCATCCTGGTCGA | |
| | GCTGGACGGCGACGTAAACGGCCACAAGTTCAGCGTGTCCGGCGA | |
| | GGGCGAGGGCGATGCCACCTACGGCAAGCTGACCCTGAAGTTCAT | |
| | CTGCACCACCGGCAAGCTGCCCGTGCCCTGGCCCACCCTCGTGACC | |
| | ACCCTGACCTACGGCGTGCAGTGCTTCAGCCGCTACCCCGACCACA | |
| | TGAAGCAGCACGACTTCTTCAAGTCCGCCATGCCCGAAGGCTACGT | |
| | CCAGGAGCGCACCATCTTCTTCAAGGACGACGGCAACTACAAGAC | |
| | CCGCGCCGAGGTGAAGTTCGAGGGCGACACCCTGGTGAACCGCAT | |
| | CGAGCTGAAGGGCATCGACTTCAAGGAGGACGGCAACATCCTGGG | |
| | GCACAAGCTGGAGTACAACTACAACAGCCACAACGTCTATATCAT | |
| | GGCCGACAAGCAGAAGAACGGCATCAAGGTGAACTTCAAGATCCG | |
| | CCACAACATCGAGGACGGCAGCGTGCAGCTCGCCGACCACTACCA | |
| | GCAGAACACCCCCATCGGCGACGGCCCCGTGCTGCTGCCCGACAA | |
| | CCACTACCTGAGCACCCAGTCCGCCCTGAGCAAAGACCCCAACGA | |
| | GAAGCGCGATCACATGGTCCTGCTGGAGTTCGTGACCGCCGCCGG | |
| | GATCACTCTCGGCATGGACGAGCTGTACAAGTAAGGATCCAAGCT | |
| | TATCGATACCGTCGACCTCGAGGGCCCAGATCTAATTCACCCCACC | |
| | AGTGCAGGCTGCCTATCAGAAAGTGGTGGCTGGTGTGGCTAATGC | |
| | CCTGGCCCACAAGTATCACTAAGCTCGCTTTCTTGCTGTCCAATTTC | |
| | TATTAAAGGTTCCTTTGTTCCCTAAGTCCAACTACTAAACTGGGGG | |
| | ATATTATGAAGGGCCTTGAGCATCTGGATTCTGCCTAATAAAAAAC | |
| | ATTTATTTTCATTGCAATGATGTATTTAAATTATTTCTGAATATTTT | |
| | ACTAAAAAGGGAATGTGGGAGGTCAGTGCATTTAAAACATAAAGA | |
| | AATGAAGAGCTAGTTCAAACCTTGGGAAAATACACTATATCTTAA | |
| | ACTCCATGAAAGAAGGTGAGGCTGCAAACAGCTAATGCACATTGG | |
| | CAACAGCCCCTGATGCCTATGCCTTATTCATCCCTCAGAAAAGGAT | |
| | TCAAGTAGAGGCTTGATTTGGAGGTTAAAGTTTTGCTATGCTGTAT | |
| | TTTACATTACTTATTGTTTTAGCTGTCCTCATGAATGTCTTTTCACT | |
| | ACCCATTTGCTTATCCTGCATCTCTCAGCCTTGACTCCACTCAGTTC | |
| | TCTTGCTTAGAGATACCACCTTTCCCCTGAAGTGTTCCTTCCATGTT | |
| | TTACGGCGAGATGGTTTCTCCTCGCCTGGCCACTCAGCCTTAGTTG | |
| | TCTCTGTTGTCTTATAGAGGTCTACTTGAAGAAGGAAAAACAGGG | |
| | GGCATGGTTTGACTGTCCTGTGAGCCCTTCTTCCCTGCCTCCCCCAC | |
| | TCACAGTGACCCGGAATCCCTCGACATGGCAGTCTAGATCATTCTT | |
| | GAAGACGAAAGGGCCTCGTGATACGCCTATTTTTATAGGTTAATGT | |
| | CATGATAATAATGGTTTCTTAGACGTCAGGTGGCACTTTTCGGGGA | |
| | AATGTGCGCGGAACCCCTATTTGTTTATTTTTCTAAATACATTCAA | |
| | ATATGTATCCGCTCATGAGACAATAACCCTGATAAATGCTTCAATA | |
| | ATATTGAAAAAGGAAGAGTATGAGTATTCAACATTTCCGTGTCGCC | |
| | CTTATTCCCTTTTTTGCGGCATTTTGCCTTCCTGTTTTTGCTCACCCA | |
| | GAAACGCTGGTGAAAGTAAAAGATGCTGAAGATCAGTTGGGTGCA | |
| | CGAGTGGGTTACATCGAACTGGATCTCAACAGCGGTAAGATCCTT | |
| | GAGAGTTTTCGCCCCGAAGAACGTTTTCCAATGATGAGCACTTTTA | |
| | AAGTTCTGCTATGTGGCGCGGTATTATCCCGTATTGACGCCGGGCA | |
| | AGAGCAACTCGGTCGCCGCATACACTATTCTCAGAATGACTTGGTT | |
| | GAGTACTCACCAGTCACAGAAAAGCATCTTACGGATGGCATGACA | |
| | GTAAGAGAATTATGCAGTGCTGCCATAACCATGAGTGATAACACT | |
| | GCGGCCAACTTACTTCTGACAACGATCGGAGGACCGAAGGAGCTA | |
| | ACCGCTTTTTTGCACAACATGGGGGATCATGTAACTCGCCTTGATC | |
| | GTTGGGAACCGGAGCTGAATGAAGCCATACCAAACGACGAGCGTG | |
| | ACACCACGATGCCTGTAGCAATGGCAACAACGTTGCGCAAACTAT | |
| | TAACTGGCGAACTACTTACTCTAGCTTCCCGGCAACAATTAATAGA | |
| | CTGGATGGAGGCGGATAAAGTTGCAGGACCACTTCTGCGCTCGGC | |
| | CCTTCCGGCTGGCTGGTTTATTGCTGATAAATCTGGAGCCGGTGAG | |
| | CGTGGGTCTCGCGGTATCATTGCAGCACTGGGGCCAGATGGTAAG | |
| | CCCTCCCGTATCGTAGTTATCTACACGACGGGGAGTCAGGCAACTA | |
| | TGGATGAACGAAATAGACAGATCGCTGAGATAGGTGCCTCACTGA | |
| | TTAAGCATTGGTAACTGTCAGACCAAGTTTACTCATATATACTTTA | |
| | GATTGATTTAAAACTTCATTTTTAATTTAAAAGGATCTAGGTGAAG | |
| | ATCCTTTTTGATAATCTCATGACCAAAATCCCTTAACGTGAGTTTTC | |
| | GTTCCACTGAGCGTCAGACCCCGTAGAAAAGATCAAAGGATCTTC | |
| | TTGAGATCCTTTTTTTCTGCGCGTAATCTGCTGCTTGCAAACAAAA | |
| | AAACCACCGCTACCAGCGGTGGTTTGTTTGCCGGATCAAGAGCTAC | |
| | CAACTCTTTTTCCGAAGGTAACTGGCTTCAGCAGAGCGCAGATACC | |
| | AAATACTGTTCTTCTAGTGTAGCCGTAGTTAGGCCACCACTTCAAG | |
| | AACTCTGTAGCACCGCCTACATACCTCGCTCTGCTAATCCTGTTAC | |
| | CAGTGGCTGCTGCCAGTGGCGATAAGTCGTGTCTTACCGGGTTGGA | |
| | CTCAAGACGATAGTTACCGGATAAGGCGCAGCGGTCGGGCTGAAC | |
| | GGGGGGTTCGTGCACACAGCCCAGCTTGGAGCGAACGACCTACAC | |
| | CGAACTGAGATACCTACAGCGTGAGCTATGAGAAAGCGCCACGCT | |
| | TCCCGAAGGGAGAAAGGCGGACAGGTATCCGGTAAGCGGCAGGG | |
| | TCGGAACAGGAGAGCGCACGAGGGAGCTTCCAGGGGGAAACGCC | |
| | TGGTATCTTTATAGTCCTGTCGGGTTTCGCCACCTCTGACTTGAGCG | |
| | TCGATTTTTGTGATGCTCGTCAGGGGGGCGGAGCCTATGGAAAAA | |
| | CGCCAGCAACGGAGATGCGCCGCGTGCGGCTGCTGGAGATGGCGG | |
| | ACGCGATGGATATGTTCTGCCAAGGGTTGGTTTGCGCATTCACAGT | |
| | TCTCCGCAAGAATTGATTGGCTCCAATTCTTGGAGTGGTGAATCCG | |
| | TTAGCGAGGTGCCGCCGGCTTCCATTCAGGTCGAGGTGGCCCGGCT | |
| | CCATGCACCGCGACGCAACGCGGGGAGGCAGACAAGGTATAGGG | |
| | CGGCGCCTACAATCCATGCCAACCCGTTCCATGTGCTCGCCGAGGC | |
| | GGCATAAATCGCCGTGACGATCAGCGGTCCAATGATCGAAGTTAG | |
| | GCTGGTAAGAGCCGCGAGCGATCCTTGAAGCTGTCCCTGATGGTC | |
| | GTCATCTACCTGCCTGGACAGCATGGCCTGCAACGCGGGCATCCCG | |
| | ATGCCGCCGGAAGCGAGAAGAATCATAATGGGGAAGGCCATCCAG | |
| | CCTCGCGTCGGGGAGCTTTTTGCAAAAGCCTAGGCCTCCAAAAAA | |
| | GCCTCCTCACTACTTCTGGAATAGCTCAGAGGCCGAGGCGGCCTCG | |
| | GCCTCTGCATAAATAAAAAAAATTAGTCAGCCATG | |
| 150 | AGCTTGGCCCATTGCATACGTTGTATCCATATCATAATATGTACAT | MA-tdMS2 |
| | TTATATTGGCTCATGTCCAACATTACCGCCATGTTGACATTGATTAT | |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCCGC | |
| | GCCTCCGTGCTGTCCGGCGGCGAGCTGGACAAGTGGGAGAAGATC | |
| | CGCCTGCGCCCCGGCGGCAAGAAGCAGTACAAGCTGAAGCACATC | |
| | GTGTGGGCCTCCCGCGAGCTGGAGCGCTTCGCCGTGAACCCCGGC | |
| | CTGCTGGAAACCTCCGAGGGCTGCCGCCAGATCCTGGGCCAGCTG | |
| | CAGCCCTCCCTGCAAACCGGCTCCGAGGAGCTGCGCTCCCTGTACA | |
| | ACACCATCGCCGTGCTGTACTGCGTGCACCAGCGCATCGACGTGA | |
| | AGGACACCAAGGAGGCCCTGGACAAGATCGAGGAGGAGCAGAAC | |
| | AAGTCCAAGAAGAAGGCCCAGCAGGCCGCCGCCGACACCGGCAA | |
| | CAACTCCCAGGTGTCCCAGAACTACCCCATCGTGCAGATGGCTTCT | |
| | AACTTTACTCAGTTCGTTCTCGTCGACAATGGCGGAACTGGCGACG | |
| | TGACTGTCGCCCCAAGCAACTTCGCTAACGGGATCGCTGAATGGAT | |
| | CAGCTCTAACTCGCGTTCACAGGCTTACAAAGTAACCTGTAGCGTT | |
| | CGTCAGAGCTCTGCGCAGAATCGCAAATACACCATCAAAGTCGAG | |
| | GTGCCTAAAGGCGCCTGGCGTTCGTACTTAAATATGGAACTAACCA | |
| | TTCCAATTTTCGCCACGAATTCCGACTGCGAGCTTATTGTTAAGGC | |
| | AATGCAAGGTCTCCTAAAAGATGGAAACCCGATTCCCTCAGCAAT | |
| | CGCAGCAAACTCCGGCATCTACGCCATGGCCAGCAACTTCACCCA | |
| | GTTCGTGCTGGTGGACAACGGCGGCACCGGCGACGTGACCGTGGC | |
| | CCCCAGCAACTTCGCCAACGGCATCGCCGAGTGGATCAGCAGCAA | |
| | CAGCAGAAGCCAGGCCTACAAGGTGACATGCAGCGTGAGACAGA | |
| | GCAGCGCCCAGAACAGAAAGTACACCATCAAGGTGGAGGTGCCCA | |
| | AGGGCGCCTGGAGAAGCTACCTGAACATGGAGCTGACCATCCCCA | |
| | TCTTCGCCACCAACAGCGACTGCGAGCTGATCGTGAAGGCCATGC | |
| | AGGGCCTGCTGAAGGACGGCAACCCCATCCCCAGCGCCATCGCCG | |
| | CCAACAGCGGCATCTACTAAGGATCCAAGCTTATCGATACCGTCG | |
| | ACCTCGAGGGCCCAGATCTAATTCACCCCACCAGTGCAGGCTGCCT | |
| | ATCAGAAAGTGGTGGCTGGTGTGGCTAATGCCCTGGCCCACAAGT | |
| | ATCACTAAGCTCGCTTTCTTGCTGTCCAATTTCTATTAAAGGTTCCT | |
| | TTGTTCCCTAAGTCCAACTACTAAACTGGGGGATATTATGAAGGGC | |
| | CTTGAGCATCTGGATTCTGCCTAATAAAAAACATTTATTTTCATTG | |
| | CAATGATGTATTTAAATTATTTCTGAATATTTTACTAAAAAGGGAA | |
| | TGTGGGAGGTCAGTGCATTTAAAACATAAAGAAATGAAGAGCTAG | |
| | TTCAAACCTTGGGAAAATACACTATATCTTAAACTCCATGAAAGAA | |
| | GGTGAGGCTGCAAACAGCTAATGCACATTGGCAACAGCCCCTGAT | |
| | GCCTATGCCTTATTCATCCCTCAGAAAAGGATTCAAGTAGAGGCTT | |
| | GATTTGGAGGTTAAAGTTTTGCTATGCTGTATTTTACATTACTTATT | |
| | GTTTTAGCTGTCCTCATGAATGTCTTTTCACTACCCATTTGCTTATC | |
| | CTGCATCTCTCAGCCTTGACTCCACTCAGTTCTCTTGCTTAGAGATA | |
| | CCACCTTTCCCCTGAAGTGTTCCTTCCATGTTTTACGGCGAGATGGT | |
| | TTCTCCTCGCCTGGCCACTCAGCCTTAGTTGTCTCTGTTGTCTTATA | |
| | GAGGTCTACTTGAAGAAGGAAAAACAGGGGGCATGGTTTGACTGT | |
| | CCTGTGAGCCCTTCTTCCCTGCCTCCCCCACTCACAGTGACCCGGA | |
| | ATCCCTCGACATGGCAGTCTAGATCATTCTTGAAGACGAAAGGGC | |
| | CTCGTGATACGCCTATTTTTATAGGTTAATGTCATGATAATAATGG | |
| | TTTCTTAGACGTCAGGTGGCACTTTTCGGGGAAATGTGCGCGGAAC | |
| | CCCTATTTGTTTATTTTTCTAAATACATTCAAATATGTATCCGCTCA | |
| | TGAGACAATAACCCTGATAAATGCTTCAATAATATTGAAAAAGGA | |
| | AGAGTATGAGTATTCAACATTTCCGTGTCGCCCTTATTCCCTTTTTT | |
| | GCGGCATTTTGCCTTCCTGTTTTTGCTCACCCAGAAACGCTGGTGA | |
| | AAGTAAAAGATGCTGAAGATCAGTTGGGTGCACGAGTGGGTTACA | |
| | TCGAACTGGATCTCAACAGCGGTAAGATCCTTGAGAGTTTTCGCCC | |
| | CGAAGAACGTTTTCCAATGATGAGCACTTTTAAAGTTCTGCTATGT | |
| | GGCGCGGTATTATCCCGTATTGACGCCGGGCAAGAGCAACTCGGT | |
| | CGCCGCATACACTATTCTCAGAATGACTTGGTTGAGTACTCACCAG | |
| | TCACAGAAAAGCATCTTACGGATGGCATGACAGTAAGAGAATTAT | |
| | GCAGTGCTGCCATAACCATGAGTGATAACACTGCGGCCAACTTACT | |
| | TCTGACAACGATCGGAGGACCGAAGGAGCTAACCGCTTTTTTGCA | |
| | CAACATGGGGGATCATGTAACTCGCCTTGATCGTTGGGAACCGGA | |
| | GCTGAATGAAGCCATACCAAACGACGAGCGTGACACCACGATGCC | |
| | TGTAGCAATGGCAACAACGTTGCGCAAACTATTAACTGGCGAACT | |
| | ACTTACTCTAGCTTCCCGGCAACAATTAATAGACTGGATGGAGGCG | |
| | GATAAAGTTGCAGGACCACTTCTGCGCTCGGCCCTTCCGGCTGGCT | |
| | GGTTTATTGCTGATAAATCTGGAGCCGGTGAGCGTGGGTCTCGCGG | |
| | TATCATTGCAGCACTGGGGCCAGATGGTAAGCCCTCCCGTATCGTA | |
| | GTTATCTACACGACGGGGAGTCAGGCAACTATGGATGAACGAAAT | |
| | AGACAGATCGCTGAGATAGGTGCCTCACTGATTAAGCATTGGTAA | |
| | CTGTCAGACCAAGTTTACTCATATATACTTTAGATTGATTTAAAAC | |
| | TTCATTTTTAATTTAAAAGGATCTAGGTGAAGATCCTTTTTGATAAT | |
| | CTCATGACCAAAATCCCTTAACGTGAGTTTTCGTTCCACTGAGCGT | |
| | CAGACCCCGTAGAAAAGATCAAAGGATCTTCTTGAGATCCTTTTTT | |
| | TCTGCGCGTAATCTGCTGCTTGCAAACAAAAAAACCACCGCTACCA | |
| | GCGGTGGTTTGTTTGCCGGATCAAGAGCTACCAACTCTTTTTCCGA | |
| | AGGTAACTGGCTTCAGCAGAGCGCAGATACCAAATACTGTTCTTCT | |
| | AGTGTAGCCGTAGTTAGGCCACCACTTCAAGAACTCTGTAGCACCG | |
| | CCTACATACCTCGCTCTGCTAATCCTGTTACCAGTGGCTGCTGCCA | |
| | GTGGCGATAAGTCGTGTCTTACCGGGTTGGACTCAAGACGATAGTT | |
| | ACCGGATAAGGCGCAGCGGTCGGGCTGAACGGGGGGTTCGTGCAC | |
| | ACAGCCCAGCTTGGAGCGAACGACCTACACCGAACTGAGATACCT | |
| | ACAGCGTGAGCTATGAGAAAGCGCCACGCTTCCCGAAGGGAGAAA | |
| | GGCGGACAGGTATCCGGTAAGCGGCAGGGTCGGAACAGGAGAGC | |
| | GCACGAGGGAGCTTCCAGGGGGAAACGCCTGGTATCTTTATAGTC | |
| | CTGTCGGGTTTCGCCACCTCTGACTTGAGCGTCGATTTTTGTGATGC | |
| | TCGTCAGGGGGGCGGAGCCTATGGAAAAACGCCAGCAACGGATGC | |
| | GCCGCGTGCGGCTGCTGGAGATGGCGGACGCGATGGATATGTTCT | |
| | GCCAAGGGTTGGTTTGCGCATTCACAGTTCTCCGCAAGAATTGATT | |
| | GGCTCCAATTCTTGGAGTGGTGAATCCGTTAGCGAGGTGCCGCCGG | |
| | CTTCCATTCAGGTCGAGGTGGCCCGGCTCCATGCACCGCGACGCAA | |
| | CGCGGGGAGGCAGACAAGGTATAGGGCGGCGCCTACAATCCATGC | |
| | CAACCCGTTCCATGTGCTCGCCGAGGCGGCATAAATCCCCGTGACG | |
| | ATCAGCGGTCCAATGATCGAAGTTAGGCTGGTAAGAGCCGCGAGC | |
| | GATCCTTGAAGCTGTCCCTGATGGTCGTCATCTACCTGCCTGGACA | |
| | GCATGGCCTGCAACGCGGGCATCCCGATGCCGCCGGAAGCGAGAA | |
| | GAATCATAATGGGGAAGGCCATCCAGCCTCGCGTCGGGGAGCTTT | |
| | TTGCAAAAGCCTAGGCCTCCAAAAAAGCCTCCTCACTACTTCTGGA | |
| | ATAGCTCAGAGGCCGAGGCGGCCTCGGCCTCTGCATAAATAAAAA | |
| | AAATTAGTCAGCCATG | |
| 151 | AGCTTGGCCCATTGCATACGTTGTATCCATATCATAATATGTACAT | VSVG-MS2_{cp} |
| | TTATATTGGCTCATGTCCAACATTACCGCCATGTTGACATTGATTAT | |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGAAGTGCCTTT | |
| | TGTACTTAGCCTTTTTATTCATTGGGGTGAATTGCAAGTTCACCATA | |
| | GTTTTTCCACACAACCAAAAAGGAAACTGGAAAAATGTTCCTTCTA | |
| | ATTACCATTATTGCCCGTCAAGCTCAGATTTAAATTGGCATAATGA | |
| | CTTAATAGGCACAGCCTTACAAGTCAAAATGCCCAAGAGTCACAA | |
| | GGCTATTCAAGCAGACGGTTGGATGTGTCATGCTTCCAAATGGGTC | |
| | ACTACTTGTGATTTCCGCTGGTATGGACCGAAGTATATAACACATT | |
| | CCATCCGATCCTTCACTCCATCTGTAGAACAATGCAAGGAAAGCAT | |
| | TGAACAAACGAAACAAGGAACTTGGCTGAATCCAGGCTTCCCTCC | |
| | TCAAAGTTGTGGATATGCAACTGTGACGGATGCCGAAGCAGTGAT | |
| | TGTCCAGGTGACTCCTCACCATGTGCTGGTTGATGAATACACAGGA | |
| | GAATGGGTTGATTCACAGTTCATCAACGGAAAATGCAGCAATTAC | |
| | ATATGCCCCACTGTCCATAACTCTACAACCTGGCATTCTGACTATA | |
| | AGGTCAAAGGGCTATGTGATTCTAACCTCATTTCCATGGACATCAC | |
| | CTTCTTCTCAGAGGACGGAGAGCTATCATCCCTGGGAAAGGAGGG | |
| | CACAGGGTTCAGAAGTAACTACTTTGCTTATGAAACTGGAGGCAA | |
| | GGCCTGCAAAATGCAATACTGCAAGCATTGGGGAGTCAGACTCCC | |
| | ATCAGGTGTCTGGTTCGAGATGGCTGATAAGGATCTCTTTGCTGCA | |
| | GCCAGATTCCCTGAATGCCCAGAAGGGTCAAGTATCTCTGCTCCAT | |
| | CTCAGACCTCAGTGGATGTAAGTCTAATTCAGGACGTTGAGAGGA | |
| | TCTTGGATTATTCCCTCTGCCAAGAAACCTGGAGCAAAATCAGAGC | |
| | GGGTCTTCCAATCTCTCCAGTGGATCTCAGCTATCTTGCTCCTAAA | |
| | AACCCAGGAACCGGTCCTGCTTTCACCATAATCAATGGTACCCTAA | |
| | AATACTTTGAGACCAGATACATCAGAGTCGATATTGCTGCTCCAAT | |
| | CCTCTCAAGAATGGTCGGAATGATCAGTGGAACTACCACAGAAAG | |
| | GGAACTGTGGGATGACTGGGCACCATATGAAGACGTGGAAATTGG | |
| | ACCCAATGGAGTTCTGAGGACCAGTTCAGGATATAAGTTTCCTTTA | |
| | TACATGATTGGACATGGTATGTTGGACTCCGATCTTCATCTTAGCT | |
| | CAAAGGCTCAGGTGTTCGAACATCCTCACATTCAAGACGCTGCTTC | |
| | GCAACTTCCTGATGATGAGAGTTTATTTTTTGGTGATACTGGGCTA | |
| | TCCAAAAATCCAATCGAGCTTGTAGAAGGTTGGTTCAGTAGTTGGA | |
| | AAAGCTCTATTGCCTCTTTTTTCTTTATCATAGGGTTAATCATTGGA | |
| | CTATTCTTGGTTCTCCGAGTTGGTATCCATCTTTGCATTAAATTAAA | |
| | GCACACCAAGAAAAGACAGATTTATACAGACATAGAGATGAACCG | |
| | ACTTGGAAAGATGGCCAGCAATTTTACGCAATTCGTGCTCGTGGAC | |
| | AACGGCGGCACGGGCGACGTGACCGTGGCCCCCAGCAACTTCGCC | |
| | AATGGCATCGCCGAATGGATCAGCAGCAACAGCAGGAGCCAGGCG | |
| | TATAAAGTTACGTGCAGCGTCAGACAGAGCAGCGCCCAGAACAGG | |
| | AAATATACGATCAAGGTCGAGGTTCCCAAGGGAGCTTGGAGGAGC | |
| | TATCTTAATATGGAGCTGACCATCCCCATCTTCGCGACAAATTCAG | |
| | ACTGCGAGCTCATCGTGAAGGCAATGCAGGGCCTCTTGAAAGATG | |
| | GCAACCCCATCCCAAGCGCAATCGCGGCCAACTCAGGAATCTACT | |
| | AAGGATCCAAGCTTATCGATACCGTCGACCTCGAGGGCCCAGATC | |
| | TAATTCACCCCACCAGTGCAGGCTGCCTATCAGAAAGTGGTGGCTG | |
| | GTGTGGCTAATGCCCTGGCCCACAAGTATCACTAAGCTCGCTTTCT | |
| | TGCTGTCCAATTTCTATTAAAGGTTCCTTTGTTCCCTAAGTCCAACT | |
| | ACTAAACTGGGGGATATTATGAAGGGCCTTGAGCATCTGGATTCTG | |
| | CCTAATAAAAAACATTTATTTTCATTGCAATGATGTATTTAAATTAT | |
| | TTCTGAATATTTTACTAAAAAGGGAATGTGGGAGGTCAGTGCATTT | |
| | AAAACATAAAGAAATGAAGAGCTAGTTCAAACCTTGGGAAAATAC | |
| | ACTATATCTTAAACTCCATGAAAGAAGGTGAGGCTGCAAACAGCT | |
| | AATGCACATTGGCAACAGCCCCTGATGCCTATGCCTTATTCATCCC | |
| | TCAGAAAAGGATTCAAGTAGAGGCTTGATTTGGAGGTTAAAGTTTT | |
| | GCTATGCTGTATTTTACATTACTTATTGTTTTAGCTGTCCTCATGAA | |
| | TGTCTTTTCACTACCCATTTGCTTATCCTGCATCTCTCAGCCTTGAC | |
| | TCCACTCAGTTCTCTTGCTTAGAGATACCACCTTTCCCCTGAAGTGT | |
| | TCCTTCCATGTTTTACGGCGAGATGGTTTCTCCTCGCCTGGCCACTC | |
| | AGCCTTAGTTGTCTCTGTTGTCTTATAGAGGTCTACTTGAAGAAGG | |
| | AAAAACAGGGGGCATGGTTTGACTGTCCTGTGAGCCCTTCTTCCCT | |
| | GCCTCCCCCACTCACAGTGACCCGGAATCCCTCGACATGGCAGTCT | |
| | AGATCATTCTTGAAGACGAAAGGGCCTCGTGATACGCCTATTTTTA | |
| | TAGGTTAATGTCATGATAATAATGGTTTCTTAGACGTCAGGTGGCA | |
| | CTTTTCGGGGAAATGTGCGCGGAACCCCTATTTGTTTATTTTTCTAA | |
| | ATACATTCAAATATGTATCCGCTCATGAGACAATAACCCTGATAAA | |
| | TGCTTCAATAATATTGAAAAAGGAAGAGTATGAGTATTCAACATTT | |
| | CCGTGTCGCCCTTATTCCCTTTTTTGCGGCATTTTGCCTTCCTGTTTT | |
| | TGCTCACCCAGAAACGCTGGTGAAAGTAAAAGATGCTGAAGATCA | |
| | GTTGGGTGCACGAGTGGGTTACATCGAACTGGATCTCAACAGCGG | |
| | TAAGATCCTTGAGAGTTTTCGCCCCGAAGAACGTTTTCCAATGATG | |
| | AGCACTTTTAAAGTTCTGCTATGTGGCGCGGTATTATCCCGTATTG | |
| | ACGCCGGGCAAGAGCAACTCGGTCGCCGCATACACTATTCTCAGA | |
| | ATGACTTGGTTGAGTACTCACCAGTCACAGAAAAGCATCTTACGG | |
| | ATGGCATGACAGTAAGAGAATTATGCAGTGCTGCCATAACCATGA | |
| | GTGATAACACTGCGGCCAACTTACTTCTGACAACGATCGGAGGAC | |
| | CGAAGGAGCTAACCGCTTTTTTGCACAACATGGGGGATCATGTAA | |
| | CTCGCCTTGATCGTTGGGAACCGGAGCTGAATGAAGCCATACCAA | |
| | ACGACGAGCGTGACACCACGATGCCTGTAGCAATGGCAACAACGT | |
| | TGCGCAAACTATTAACTGGCGAACTACTTACTCTAGCTTCCCGGCA | |
| | ACAATTAATAGACTGGATGGAGGCGGATAAAGTTGCAGGACCACT | |
| | TCTGCGCTCGGCCCTTCCGGCTGGCTGGTTTATTGCTGATAAATCT | |
| | GGAGCCGGTGAGCGTGGGTCTCGCGGTATCATTGCAGCACTGGGG | |
| | CCAGATGGTAAGCCCTCCCGTATCGTAGTTATCTACACGACGGGGA | |
| | GTCAGGCAACTATGGATGAACGAAATAGACAGATCGCTGAGATAG | |
| | GTGCCTCACTGATTAAGCATTGGTAACTGTCAGACCAAGTTTACTC | |
| | ATATATACTTTAGATTGATTTAAAACTTCATTTTTAATTTAAAAGGA | |
| | TCTAGGTGAAGATCCTTTTTGATAATCTCATGACCAAAATCCCTTA | |
| | ACGTGAGTTTTCGTTCCACTGAGCGTCAGACCCCGTAGAAAAGATC | |
| | AAAGGATCTTCTTGAGATCCTTTTTTTCTGCGCGTAATCTGCTGCTT | |
| | GCAAACAAAAAAACCACCGCTACCAGCGGTGGTTTGTTTGCCGGA | |
| | TCAAGAGCTACCAACTCTTTTTCCGAAGGTAACTGGCTTCAGCAGA | |
| | GCGCAGATACCAAATACTGTTCTTCTAGTGTAGCCGTAGTTAGGCC | |
| | ACCACTTCAAGAACTCTGTAGCACCGCCTACATACCTCGCTCTGCT | |
| | AATCCTGTTACCAGTGGCTGCTGCCAGTGGCGATAAGTCGTGTCTT | |
| | ACCGGGTTGGACTCAAGACGATAGTTACCGGATAAGGCGCAGCGG | |
| | TCGGGCTGAACGGGGGGTTCGTGCACACAGCCCAGCTTGGAGCGA | |
| | ACGACCTACACCGAACTGAGATACCTACAGCGTGAGCTATGAGAA | |
| | AGCGCCACGCTTCCCGAAGGGAGAAAGGCGGACAGGTATCCGGTA | |
| | AGCGGCAGGGTCGGAACAGGAGAGCGCACGAGGGAGCTTCCAGG | |
| | GGGAAACGCCTGGTATCTTTATAGTCCTGTCGGGTTTCGCCACCTC | |
| | TGACTTGAGCGTCGATTTTTGTGATGCTCGTCAGGGGGGCGGAGCC | |
| | TATGGAAAAACGCCAGCAACGGAGATGCGCCGCGTGCGGCTGCTG | |
| | GAGATGGCGGACGCGATGGATATGTTCTGCCAAGGGTTGGTTTGC | |
| | GCATTCACAGTTCTCCGCAAGAATTGATTGGCTCCAATTCTTGGAG | |
| | TGGTGAATCCGTTAGCGAGGTGCCGCCGGCTTCCATTCAGGTCGAG | |
| | GTGGCCCGGCTCCATGCACCGCGACGCAACGCGGGGAGGCAGACA | |
| | AGGTATAGGGCGGCGCCTACAATCCATGCCAACCCGTTCCATGTGC | |
| | TCGCCGAGGCGGCATAAATCGCCGTGACGATCAGCGGTCCAATGA | |
| | TCGAAGTTAGGCTGGTAAGAGCCGCGAGCGATCCTTGAAGCTGTC | |
| | CCTGATGGTCGTCATCTACCTGCCTGGACAGCATGGCCTGCAACGC | |
| | GGGCATCCCGATGCCGCCGGAAGCGAGAAGAATCATAATGGGGAA | |
| | GGCCATCCAGCCTCGCGTCGGGGAGCTTTTTGCAAAAGCCTAGGCC | |
| | TCCAAAAAAGCCTCCTCACTACTTCTGGAATAGCTCAGAGGCCGA | |
| | GGCGGCCTCGGCCTCTGCATAAATAAAAAAAATTAGTCAGCCATG | |
| 152 | AGCTTGGCCCATTGCATACGTTGTATCCATATCATAATATGTACAT | VSVG-td- |
| | TTATATTGGCTCATGTCCAACATTACCGCCATGTTGACATTGATTAT | MS2_{cp} |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGAAGTGCCTTT | |
| | TGTACTTAGCCTTTTTATTCATTGGGGTGAATTGCAAGTTCACCATA | |
| | GTTTTTCCACACAACCAAAAAGGAAACTGGAAAAATGTTCCTTCTA | |
| | ATTACCATTATTGCCCGTCAAGCTCAGATTTAAATTGGCATAATGA | |
| | CTTAATAGGCACAGCCTTACAAGTCAAAATGCCCAAGAGTCACAA | |
| | GGCTATTCAAGCAGACGGTTGGATGTGTCATGCTTCCAAATGGGTC | |
| | ACTACTTGTGATTTCCGCTGGTATGGACCGAAGTATATAACACATT | |
| | CCATCCGATCCTTCACTCCATCTGTAGAACAATGCAAGGAAAGCAT | |
| | TGAACAAACGAAACAAGGAACTTGGCTGAATCCAGGCTTCCCTCC | |
| | TCAAAGTTGTGGATATGCAACTGTGACGGATGCCGAAGCAGTGAT | |
| | TGTCCAGGTGACTCCTCACCATGTGCTGGTTGATGAATACACAGGA | |
| | GAATGGGTTGATTCACAGTTCATCAACGGAAAATGCAGCAATTAC | |
| | ATATGCCCCACTGTCCATAACTCTACAACCTGGCATTCTGACTATA | |
| | AGGTCAAAGGGCTATGTGATTCTAACCTCATTTCCATGGACATCAC | |
| | CTTCTTCTCAGAGGACGGAGAGCTATCATCCCTGGGAAAGGAGGG | |
| | CACAGGGTTCAGAAGTAACTACTTTGCTTATGAAACTGGAGGCAA | |
| | GGCCTGCAAAATGCAATACTGCAAGCATTGGGGAGTCAGACTCCC | |
| | ATCAGGTGTCTGGTTCGAGATGGCTGATAAGGATCTCTTTGCTGCA | |
| | GCCAGATTCCCTGAATGCCCAGAAGGGTCAAGTATCTCTGCTCCAT | |
| | CTCAGACCTCAGTGGATGTAAGTCTAATTCAGGACGTTGAGAGGA | |
| | TCTTGGATTATTCCCTCTGCCAAGAAACCTGGAGCAAAATCAGAGC | |
| | GGGTCTTCCAATCTCTCCAGTGGATCTCAGCTATCTTGCTCCTAAA | |
| | AACCCAGGAACCGGTCCTGCTTTCACCATAATCAATGGTACCCTAA | |
| | AATACTTTGAGACCAGATACATCAGAGTCGATATTGCTGCTCCAAT | |
| | CCTCTCAAGAATGGTCGGAATGATCAGTGGAACTACCACAGAAAG | |
| | GGAACTGTGGGATGACTGGGCACCATATGAAGACGTGGAAATTGG | |
| | ACCCAATGGAGTTCTGAGGACCAGTTCAGGATATAAGTTTCCTTTA | |
| | TACATGATTGGACATGGTATGTTGGACTCCGATCTTCATCTTAGCT | |
| | CAAAGGCTCAGGTGTTCGAACATCCTCACATTCAAGACGCTGCTTC | |
| | GCAACTTCCTGATGATGAGAGTTTATTTTTTGGTGATACTGGGCTA | |
| | TCCAAAAATCCAATCGAGCTTGTAGAAGGTTGGTTCAGTAGTTGGA | |
| | AAAGCTCTATTGCCTCTTTTTTCTTTATCATAGGGTTAATCATTGGA | |
| | CTATTCTTGGTTCTCCGAGTTGGTATCCATCTTTGCATTAAATTAAA | |
| | GCACACCAAGAAAAGACAGATTTATACAGACATAGAGATGAACCG | |
| | ACTTGGAAAGATGGCTTCTAACTTTACTCAGTTCGTTCTCGTCGAC | |
| | AATGGCGGAACTGGCGACGTGACTGTCGCCCCAAGCAACTTCGCT | |
| | AACGGGATCGCTGAATGGATCAGCTCTAACTCGCGTTCACAGGCTT | |
| | ACAAAGTAACCTGTAGCGTTCGTCAGAGCTCTGCGCAGAATCGCA | |
| | AATACACCATCAAAGTCGAGGTGCCTAAAGGCGCCTGGCGTTCGT | |
| | ACTTAAATATGGAACTAACCATTCCAATTTTCGCCACGAATTCCGA | |
| | CTGCGAGCTTATTGTTAAGGCAATGCAAGGTCTCCTAAAAGATGG | |
| | AAACCCGATTCCCTCAGCAATCGCAGCAAACTCCGGCATCTACGCC | |
| | ATGGCCAGCAACTTCACCCAGTTCGTGCTGGTGGACAACGGCGGC | |
| | ACCGGCGACGTGACCGTGGCCCCCAGCAACTTCGCCAACGGCATC | |
| | GCCGAGTGGATCAGCAGCAACAGCAGAAGCCAGGCCTACAAGGTG | |
| | ACCTGCAGCGTGAGACAGAGCAGCGCCCAGAACAGAAAGTACACC | |
| | ATCAAGGTGGAGGTGCCCAAGGGCGCCTGGAGAAGCTACCTGAAC | |
| | ATGGAGCTGACCATCCCCATCTTCGCCACCAACAGCGACTGCGAG | |
| | CTGATCGTGAAGGCCATGCAGGGCCTGCTGAAGGACGGCAACCCC | |
| | ATCCCCAGCGCCATCGCCGCCAACAGCGGCATCTACTAAGGATCC | |
| | AAGCTTATCGATACCGTCGACCTCGAGGGCCCAGATCTAATTCACC | |
| | CCACCAGTGCAGGCTGCCTATCAGAAAGTGGTGGCTGGTGTGGCT | |
| | AATGCCCTGGCCCACAAGTATCACTAAGCTCGCTTTCTTGCTGTCC | |
| | AATTTCTATTAAAGGTTCCTTTGTTCCCTAAGTCCAACTACTAAACT | |
| | GGGGGATATTATGAAGGGCCTTGAGCATCTGGATTCTGCCTAATAA | |
| | AAAACATTTATTTTCATTGCAATGATGTATTTAAATTATTTCTGAAT | |
| | ATTTTACTAAAAAGGGAATGTGGGAGGTCAGTGCATTTAAAACAT | |
| | AAAGAAATGAAGAGCTAGTTCAAACCTTGGGAAAATACACTATAT | |
| | CTTAAACTCCATGAAAGAAGGTGAGGCTGCAAACAGCTAATGCAC | |
| | ATTGGCAACAGCCCCTGATGCCTATGCCTTATTCATCCCTCAGAAA | |
| | AGGATTCAAGTAGAGGCTTGATTTGGAGGTTAAAGTTTTGCTATGC | |
| | TGTATTTTACATTACTTATTGTTTTAGCTGTCCTCATGAATGTCTTTT | |
| | CACTACCCATTTGCTTATCCTGCATCTCTCAGCCTTGACTCCACTCA | |
| | GTTCTCTTGCTTAGAGATACCACCTTTCCCCTGAAGTGTTCCTTCCA | |
| | TGTTTTACGGCGAGATGGTTTCTCCTCGCCTGGCCACTCAGCCTTA | |
| | GTTGTCTCTGTTGTCTTATAGAGGTCTACTTGAAGAAGGAAAAACA | |
| | GGGGGCATGGTTTGACTGTCCTGTGAGCCCTTCTTCCCTGCCTCCC | |
| | CCACTCACAGTGACCCGGAATCCCTCGACATGGCAGTCTAGATCAT | |
| | TCTTGAAGACGAAAGGGCCTCGTGATACGCCTATTTTTATAGGTTA | |
| | ATGTCATGATAATAATGGTTTCTTAGACGTCAGGTGGCACTTTTCG | |
| | GGGAAATGTGCGCGGAACCCCTATTTGTTTATTTTTCTAAATACAT | |
| | TCAAATATGTATCCGCTCATGAGACAATAACCCTGATAAATGCTTC | |
| | AATAATATTGAAAAAGGAAGAGTATGAGTATTCAACATTTCCGTGT | |
| | CGCCCTTATTCCCTTTTTTGCGGCATTTTGCCTTCCTGTTTTTGCTCA | |
| | CCCAGAAACGCTGGTGAAAGTAAAAGATGCTGAAGATCAGTTGGG | |
| | TGCACGAGTGGGTTACATCGAACTGGATCTCAACAGCGGTAAGAT | |
| | CCTTGAGAGTTTTCGCCCCGAAGAACGTTTTCCAATGATGAGCACT | |
| | TTTAAAGTTCTGCTATGTGGCGCGGTATTATCCCGTATTGACGCCG | |
| | GGCAAGAGCAACTCGGTCGCCGCATACACTATTCTCAGAATGACTT | |
| | GGTTGAGTACTCACCAGTCACAGAAAAGCATCTTACGGATGGCAT | |
| | GACAGTAAGAGAATTATGCAGTGCTGCCATAACCATGAGTGATAA | |
| | CACTGCGGCCAACTTACTTCTGACAACGATCGGAGGACCGAAGGA | |
| | GCTAACCGCTTTTTTGCACAACATGGGGGATCATGTAACTCGCCTT | |
| | GATCGTTGGGAACCGGAGCTGAATGAAGCCATACCAAACGACGAG | |
| | CGTGACACCACGATGCCTGTAGCAATGGCAACAACGTTGCGCAAA | |
| | CTATTAACTGGCGAACTACTTACTCTAGCTTCCCGGCAACAATTAA | |
| | TAGACTGGATGGAGGCGGATAAAGTTGCAGGACCACTTCTGCGCT | |
| | CGGCCCTTCCGGCTGGCTGGTTTATTGCTGATAAATCTGGAGCCGG | |
| | TGAGCGTGGGTCTCGCGGTATCATTGCAGCACTGGGGCCAGATGG | |
| | TAAGCCCTCCCGTATCGTAGTTATCTACACGACGGGGAGTCAGGCA | |
| | ACTATGGATGAACGAAATAGACAGATCGCTGAGATAGGTGCCTCA | |
| | CTGATTAAGCATTGGTAACTGTCAGACCAAGTTTACTCATATATAC | |
| | TTTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGAGATGCGCCGCGTGCGGCTGCTGGAGATGG | |
| | CGGACGCGATGGATATGTTCTGCCAAGGGTTGGTTTGCGCATTCAC | |
| | AGTTCTCCGCAAGAATTGATTGGCTCCAATTCTTGGAGTGGTGAAT | |
| | CCGTTAGCGAGGTGCCGCCGGCTTCCATTCAGGTCGAGGTGGCCCG | |
| | GCTCCATGCACCGCGACGCAACGCGGGGAGGCAGACAAGGTATAG | |
| | GGCGGCGCCTACAATCCATGCCAACCCGTTCCATGTGCTCGCCGAG | |
| | GCGGCATAAATCGCCGTGACGATCAGCGGTCCAATGATCGAAGTT | |
| | AGGCTGGTAAGAGCCGCGAGCGATCCTTGAAGCTGTCCCTGATGG | |
| | TCGTCATCTACCTGCCTGGACAGCATGGCCTGCAACGCGGGCATCC | |
| | CGATGCCGCCGGAAGCGAGAAGAATCATAATGGGGAAGGCCATCC | |
| | AGCCTCGCGTCGGGGAGCTTTTTGCAAAAGCCTAGGCCTCCAAAA | |
| | AAGCCTCCTCACTACTTCTGGAATAGCTCAGAGGCCGAGGCGGCCT | |
| | CGGCCTCTGCATAAATAAAAAAAATTAGTCAGCCATG | |
| 153 | AGCTTGGCCCATTGCATACGTTGTATCCATATCATAATATGTACAT | MA (N-term)- |
| | TTATATTGGCTCATGTCCAACATTACCGCCATGTTGACATTGATTAT | λN |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCCGC | |
| | GCCTCCGTGCTGTCCGGCGGCGAGCTGGACAAGTGGGAGAAGATC | |
| | CGCCTGCGCCCCGGCGGCAAGAAGCAGTACAAGCTGAAGCACATC | |
| | GTGTGGGCCTCCCGCGAGCTGGAGCGCTTCGCCGTGAACCCCGGC | |
| | CTGCTGGAAACCTCCGAGGGCTGCCGCCAGATCCTGGGCCAGCTG | |
| | CAGCCCTCCCTGCAAACCGGCTCCGAGGAGCTGCGCTCCCTGTACA | |
| | ACACCATCGCCGTGCTGTACTGCGTGCACCAGCGCATCGACGTGA | |
| | AGGACACCAAGGAGGCCCTGGACAAGATCGAGGAGGAGCAGAAC | |
| | AAGTCCAAGAAGAAGGCCCAGCAGGCCGCCGCCGACACCGGCAA | |
| | CAACTCCCAGGTGTCCCAGAACTACCCCATCGTGCAGATGGACGC | |
| | CCAGACCAGGAGGAGGGAAAGGAGGGCAGAGAAGCAGGCCCAAT | |
| | GGAAGGCCGCCAACTAAGGATCCAAGCTTATCGATACCGTCGACC | |
| | TCGAGGGCCCAGATCTAATTCACCCCACCAGTGCAGGCTGCCTATC | |
| | AGAAAGTGGTGGCTGGTGTGGCTAATGCCCTGGCCCACAAGTATC | |
| | ACTAAGCTCGCTTTCTTGCTGTCCAATTTCTATTAAAGGTTCCTTTG | |
| | TTCCCTAAGTCCAACTACTAAACTGGGGGATATTATGAAGGGCCTT | |
| | GAGCATCTGGATTCTGCCTAATAAAAAACATTTATTTTCATTGCAA | |
| | TGATGTATTTAAATTATTTCTGAATATTTTACTAAAAAGGGAATGT | |
| | GGGAGGTCAGTGCATTTAAAACATAAAGAAATGAAGAGCTAGTTC | |
| | AAACCTTGGGAAAATACACTATATCTTAAACTCCATGAAAGAAGG | |
| | TGAGGCTGCAAACAGCTAATGCACATTGGCAACAGCCCCTGATGC | |
| | CTATGCCTTATTCATCCCTCAGAAAAGGATTCAAGTAGAGGCTTGA | |
| | TTTGGAGGTTAAAGTTTTGCTATGCTGTATTTTACATTACTTATTGT | |
| | TTTAGCTGTCCTCATGAATGTCTTTTCACTACCCATTTGCTTATCCT | |
| | GCATCTCTCAGCCTTGACTCCACTCAGTTCTCTTGCTTAGAGATACC | |
| | ACCTTTCCCCTGAAGTGTTCCTTCCATGTTTTACGGCGAGATGGTTT | |
| | CTCCTCGCCTGGCCACTCAGCCTTAGTTGTCTCTGTTGTCTTATAGA | |
| | GGTCTACTTGAAGAAGGAAAAACAGGGGGCATGGTTTGACTGTCC | |
| | TGTGAGCCCTTCTTCCCTGCCTCCCCCACTCACAGTGACCCGGAAT | |
| | CCCTCGACATGGCAGTCTAGATCATTCTTGAAGACGAAAGGGCCTC | |
| | GTGATACGCCTATTTTTATAGGTTAATGTCATGATAATAATGGTTTC | |
| | TTAGACGTCAGGTGGCACTTTTCGGGGAAATGTGCGCGGAACCCCT | |
| | ATTTGTTTATTTTTCTAAATACATTCAAATATGTATCCGCTCATGAG | |
| | ACAATAACCCTGATAAATGCTTCAATAATATTGAAAAAGGAAGAG | |
| | TATGAGTATTCAACATTTCCGTGTCGCCCTTATTCCCTTTTTTGCGG | |
| | CATTTTGCCTTCCTGTTTTTGCTCACCCAGAAACGCTGGTGAAAGT | |
| | AAAAGATGCTGAAGATCAGTTGGGTGCACGAGTGGGTTACATCGA | |
| | ACTGGATCTCAACAGCGGTAAGATCCTTGAGAGTTTTCGCCCCGAA | |
| | GAACGTTTTCCAATGATGAGCACTTTTAAAGTTCTGCTATGTGGCG | |
| | CGGTATTATCCCGTATTGACGCCGGGCAAGAGCAACTCGGTCGCC | |
| | GCATACACTATTCTCAGAATGACTTGGTTGAGTACTCACCAGTCAC | |
| | AGAAAAGCATCTTACGGATGGCATGACAGTAAGAGAATTATGCAG | |
| | TGCTGCCATAACCATGAGTGATAACACTGCGGCCAACTTACTTCTG | |
| | ACAACGATCGGAGGACCGAAGGAGCTAACCGCTTTTTTGCACAAC | |
| | ATGGGGGATCATGTAACTCGCCTTGATCGTTGGGAACCGGAGCTG | |
| | AATGAAGCCATACCAAACGACGAGCGTGACACCACGATGCCTGTA | |
| | GCAATGGCAACAACGTTGCGCAAACTATTAACTGGCGAACTACTT | |
| | ACTCTAGCTTCCCGGCAACAATTAATAGACTGGATGGAGGCGGAT | |
| | AAAGTTGCAGGACCACTTCTGCGCTCGGCCCTTCCGGCTGGCTGGT | |
| | TTATTGCTGATAAATCTGGAGCCGGTGAGCGTGGGTCTCGCGGTAT | |
| | CATTGCAGCACTGGGGCCAGATGGTAAGCCCTCCCGTATCGTAGTT | |
| | ATCTACACGACGGGGAGTCAGGCAACTATGGATGAACGAAATAGA | |
| | CAGATCGCTGAGATAGGTGCCTCACTGATTAAGCATTGGTAACTGT | |
| | CAGACCAAGTTTACTCATATATACTTTAGATTGATTTAAAACTTCA | |
| | TTTTTAATTTAAAAGGATCTAGGTGAAGATCCTTTTTGATAATCTCA | |
| | TGACCAAAATCCCTTAACGTGAGTTTTCGTTCCACTGAGCGTCAGA | |
| | CCCCGTAGAAAAGATCAAAGGATCTTCTTGAGATCCTTTTTTTCTG | |
| | CGCGTAATCTGCTGCTTGCAAACAAAAAAACCACCGCTACCAGCG | |
| | GTGGTTTGTTTGCCGGATCAAGAGCTACCAACTCTTTTTCCGAAGG | |
| | TAACTGGCTTCAGCAGAGCGCAGATACCAAATACTGTTCTTCTAGT | |
| | GTAGCCGTAGTTAGGCCACCACTTCAAGAACTCTGTAGCACCGCCT | |
| | ACATACCTCGCTCTGCTAATCCTGTTACCAGTGGCTGCTGCCAGTG | |
| | GCGATAAGTCGTGTCTTACCGGGTTGGACTCAAGACGATAGTTACC | |
| | GGATAAGGCGCAGCGGTCGGGCTGAACGGGGGGTTCGTGCACACA | |
| | GCCCAGCTTGGAGCGAACGACCTACACCGAACTGAGATACCTACA | |
| | GCGTGAGCTATGAGAAAGCGCCACGCTTCCCGAAGGGAGAAAGGC | |
| | GGACAGGTATCCGGTAAGCGGCAGGGTCGGAACAGGAGAGCGCA | |
| | CGAGGGAGCTTCCAGGGGGAAACGCCTGGTATCTTTATAGTCCTGT | |
| | CGGGTTTCGCCACCTCTGACTTGAGCGTCGATTTTTGTGATGCTCGT | |
| | CAGGGGGGCGGAGCCTATGGAAAAACGCCAGCAACGGATGCGCC | |
| | GCGTGCGGCTGCTGGAGATGGCGGACGCGATGGATATGTTCTGCC | |
| | AAGGGTTGGTTTGCGCATTCACAGTTCTCCGCAAGAATTGATTGGC | |
| | TCCAATTCTTGGAGTGGTGAATCCGTTAGCGAGGTGCCGCCGGCTT | |
| | CCATTCAGGTCGAGGTGGCCCGGCTCCATGCACCGCGACGCAACG | |
| | CGGGGAGGCAGACAAGGTATAGGGCGGCGCCTACAATCCATGCCA | |
| | ACCCGTTCCATGTGCTCGCCGAGGCGGCATAAATCCCCGTGACGAT | |
| | CAGCGGTCCAATGATCGAAGTTAGGCTGGTAAGAGCCGCGAGCGA | |
| | TCCTTGAAGCTGTCCCTGATGGTCGTCATCTACCTGCCTGGACAGC | |
| | ATGGCCTGCAACGCGGGCATCCCGATGCCGCCGGAAGCGAGAAGA | |
| | ATCATAATGGGGAAGGCCATCCAGCCTCGCGTCGGGGAGCTTTTTG | |
| | CAAAAGCCTAGGCCTCCAAAAAAGCCTCCTCACTACTTCTGGAATA | |
| | GCTCAGAGGCCGAGGCGGCCTCGGCCTCTGCATAAATAAAAAAAA | |
| | TTAGTCAGCCATG | |
| 154 | AGCTTGGCCCATTGCATACGTTGTATCCATATCATAATATGTACAT | MA (N-term)- |
| | TTATATTGGCTCATGTCCAACATTACCGCCATGTTGACATTGATTAT | mutλN |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCCGC | |
| | GCCTCCGTGCTGTCCGGCGGCGAGCTGGACAAGTGGGAGAAGATC | |
| | CGCCTGCGCCCCGGCGGCAAGAAGCAGTACAAGCTGAAGCACATC | |
| | GTGTGGGCCTCCCGCGAGCTGGAGCGCTTCGCCGTGAACCCCGGC | |
| | CTGCTGGAAACCTCCGAGGGCTGCCGCCAGATCCTGGGCCAGCTG | |
| | CAGCCCTCCCTGCAAACCGGCTCCGAGGAGCTGCGCTCCCTGTACA | |
| | ACACCATCGCCGTGCTGTACTGCGTGCACCAGCGCATCGACGTGA | |
| | AGGACACCAAGGAGGCCCTGGACAAGATCGAGGAGGAGCAGAAC | |
| | AAGTCCAAGAAGAAGGCCCAGCAGGCCGCCGCCGACACCGGCAA | |
| | CAACTCCCAGGTGTCCCAGAACTACCCCATCGTGCAGGGAAACGC | |
| | CCGGACCAGGAGGAGGGAAAGGAGGGCAGAGAAGCAGGCCCAAT | |
| | GGAAGGCCGCCAACTAAGGATCCAAGCTTATCGATACCGTCGACC | |
| | TCGAGGGCCCAGATCTAATTCACCCCACCAGTGCAGGCTGCCTATC | |
| | AGAAAGTGGTGGCTGGTGTGGCTAATGCCCTGGCCCACAAGTATC | |
| | ACTAAGCTCGCTTTCTTGCTGTCCAATTTCTATTAAAGGTTCCTTTG | |
| | TTCCCTAAGTCCAACTACTAAACTGGGGGATATTATGAAGGGCCTT | |
| | GAGCATCTGGATTCTGCCTAATAAAAAACATTTATTTTCATTGCAA | |
| | TGATGTATTTAAATTATTTCTGAATATTTTACTAAAAAGGGAATGT | |
| | GGGAGGTCAGTGCATTTAAAACATAAAGAAATGAAGAGCTAGTTC | |
| | AAACCTTGGGAAAATACACTATATCTTAAACTCCATGAAAGAAGG | |
| | TGAGGCTGCAAACAGCTAATGCACATTGGCAACAGCCCCTGATGC | |
| | CTATGCCTTATTCATCCCTCAGAAAAGGATTCAAGTAGAGGCTTGA | |
| | TTTGGAGGTTAAAGTTTTGCTATGCTGTATTTTACATTACTTATTGT | |
| | TTTAGCTGTCCTCATGAATGTCTTTTCACTACCCATTTGCTTATCCT | |
| | GCATCTCTCAGCCTTGACTCCACTCAGTTCTCTTGCTTAGAGATACC | |
| | ACCTTTCCCCTGAAGTGTTCCTTCCATGTTTTACGGCGAGATGGTTT | |
| | CTCCTCGCCTGGCCACTCAGCCTTAGTTGTCTCTGTTGTCTTATAGA | |
| | GGTCTACTTGAAGAAGGAAAAACAGGGGGCATGGTTTGACTGTCC | |
| | TGTGAGCCCTTCTTCCCTGCCTCCCCCACTCACAGTGACCCGGAAT | |
| | CCCTCGACATGGCAGTCTAGATCATTCTTGAAGACGAAAGGGCCTC | |
| | GTGATACGCCTATTTTTATAGGTTAATGTCATGATAATAATGGTTTC | |
| | TTAGACGTCAGGTGGCACTTTTCGGGGAAATGTGCGCGGAACCCCT | |
| | ATTTGTTTATTTTTCTAAATACATTCAAATATGTATCCGCTCATGAG | |
| | ACAATAACCCTGATAAATGCTTCAATAATATTGAAAAAGGAAGAG | |
| | TATGAGTATTCAACATTTCCGTGTCGCCCTTATTCCCTTTTTTGCGG | |
| | CATTTTGCCTTCCTGTTTTTGCTCACCCAGAAACGCTGGTGAAAGT | |
| | AAAAGATGCTGAAGATCAGTTGGGTGCACGAGTGGGTTACATCGA | |
| | ACTGGATCTCAACAGCGGTAAGATCCTTGAGAGTTTTCGCCCCGAA | |
| | GAACGTTTTCCAATGATGAGCACTTTTAAAGTTCTGCTATGTGGCG | |
| | CGGTATTATCCCGTATTGACGCCGGGCAAGAGCAACTCGGTCGCC | |
| | GCATACACTATTCTCAGAATGACTTGGTTGAGTACTCACCAGTCAC | |
| | AGAAAAGCATCTTACGGATGGCATGACAGTAAGAGAATTATGCAG | |
| | TGCTGCCATAACCATGAGTGATAACACTGCGGCCAACTTACTTCTG | |
| | ACAACGATCGGAGGACCGAAGGAGCTAACCGCTTTTTTGCACAAC | |
| | ATGGGGGATCATGTAACTCGCCTTGATCGTTGGGAACCGGAGCTG | |
| | AATGAAGCCATACCAAACGACGAGCGTGACACCACGATGCCTGTA | |
| | GCAATGGCAACAACGTTGCGCAAACTATTAACTGGCGAACTACTT | |
| | ACTCTAGCTTCCCGGCAACAATTAATAGACTGGATGGAGGCGGAT | |
| | AAAGTTGCAGGACCACTTCTGCGCTCGGCCCTTCCGGCTGGCTGGT | |
| | TTATTGCTGATAAATCTGGAGCCGGTGAGCGTGGGTCTCGCGGTAT | |
| | CATTGCAGCACTGGGGCCAGATGGTAAGCCCTCCCGTATCGTAGTT | |
| | ATCTACACGACGGGGAGTCAGGCAACTATGGATGAACGAAATAGA | |
| | CAGATCGCTGAGATAGGTGCCTCACTGATTAAGCATTGGTAACTGT | |
| | CAGACCAAGTTTACTCATATATACTTTAGATTGATTTAAAACTTCA | |
| | TTTTTAATTTAAAAGGATCTAGGTGAAGATCCTTTTTGATAATCTCA | |
| | TGACCAAAATCCCTTAACGTGAGTTTTCGTTCCACTGAGCGTCAGA | |
| | CCCCGTAGAAAAGATCAAAGGATCTTCTTGAGATCCTTTTTTTCTG | |
| | CGCGTAATCTGCTGCTTGCAAACAAAAAAACCACCGCTACCAGCG | |
| | GTGGTTTGTTTGCCGGATCAAGAGCTACCAACTCTTTTTCCGAAGG | |
| | TAACTGGCTTCAGCAGAGCGCAGATACCAAATACTGTTCTTCTAGT | |
| | GTAGCCGTAGTTAGGCCACCACTTCAAGAACTCTGTAGCACCGCCT | |
| | ACATACCTCGCTCTGCTAATCCTGTTACCAGTGGCTGCTGCCAGTG | |
| | GCGATAAGTCGTGTCTTACCGGGTTGGACTCAAGACGATAGTTACC | |
| | GGATAAGGCGCAGCGGTCGGGCTGAACGGGGGGTTCGTGCACACA | |
| | GCCCAGCTTGGAGCGAACGACCTACACCGAACTGAGATACCTACA | |
| | GCGTGAGCTATGAGAAAGCGCCACGCTTCCCGAAGGGAGAAAGGC | |
| | GGACAGGTATCCGGTAAGCGGCAGGGTCGGAACAGGAGAGCGCA | |
| | CGAGGGAGCTTCCAGGGGGAAACGCCTGGTATCTTTATAGTCCTGT | |
| | CGGGTTTCGCCACCTCTGACTTGAGCGTCGATTTTTGTGATGCTCGT | |
| | CAGGGGGGCGGAGCCTATGGAAAAACGCCAGCAACGGATGCGCC | |
| | GCGTGCGGCTGCTGGAGATGGCGGACGCGATGGATATGTTCTGCC | |
| | AAGGGTTGGTTTGCGCATTCACAGTTCTCCGCAAGAATTGATTGGC | |
| | TCCAATTCTTGGAGTGGTGAATCCGTTAGCGAGGTGCCGCCGGCTT | |
| | CCATTCAGGTCGAGGTGGCCCGGCTCCATGCACCGCGACGCAACG | |
| | CGGGGAGGCAGACAAGGTATAGGGCGGCGCCTACAATCCATGCCA | |
| | ACCCGTTCCATGTGCTCGCCGAGGCGGCATAAATCCCCGTGACGAT | |
| | CAGCGGTCCAATGATCGAAGTTAGGCTGGTAAGAGCCGCGAGCGA | |
| | TCCTTGAAGCTGTCCCTGATGGTCGTCATCTACCTGCCTGGACAGC | |
| | ATGGCCTGCAACGCGGGCATCCCGATGCCGCCGGAAGCGAGAAGA | |
| | ATCATAATGGGGAAGGCCATCCAGCCTCGCGTCGGGGAGCTTTTTG | |
| | CAAAAGCCTAGGCCTCCAAAAAAGCCTCCTCACTACTTCTGGAATA | |
| | GCTCAGAGGCCGAGGCGGCCTCGGCCTCTGCATAAATAAAAAAAA | |
| | TTAGTCAGCCATG | |
| 155 | AGCTTGGCCCATTGCATACGTTGTATCCATATCATAATATGTACAT | VSVG-λN |
| | TTATATTGGCTCATGTCCAACATTACCGCCATGTTGACATTGATTAT | |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGAAGTGCCTTT | |
| | TGTACTTAGCCTTTTTATTCATTGGGGTGAATTGCAAGTTCACCATA | |
| | GTTTTTCCACACAACCAAAAAGGAAACTGGAAAAATGTTCCTTCTA | |
| | ATTACCATTATTGCCCGTCAAGCTCAGATTTAAATTGGCATAATGA | |
| | CTTAATAGGCACAGCCTTACAAGTCAAAATGCCCAAGAGTCACAA | |
| | GGCTATTCAAGCAGACGGTTGGATGTGTCATGCTTCCAAATGGGTC | |
| | ACTACTTGTGATTTCCGCTGGTATGGACCGAAGTATATAACACATT | |
| | CCATCCGATCCTTCACTCCATCTGTAGAACAATGCAAGGAAAGCAT | |
| | TGAACAAACGAAACAAGGAACTTGGCTGAATCCAGGCTTCCCTCC | |
| | TCAAAGTTGTGGATATGCAACTGTGACGGATGCCGAAGCAGTGAT | |
| | TGTCCAGGTGACTCCTCACCATGTGCTGGTTGATGAATACACAGGA | |
| | GAATGGGTTGATTCACAGTTCATCAACGGAAAATGCAGCAATTAC | |
| | ATATGCCCCACTGTCCATAACTCTACAACCTGGCATTCTGACTATA | |
| | AGGTCAAAGGGCTATGTGATTCTAACCTCATTTCCATGGACATCAC | |
| | CTTCTTCTCAGAGGACGGAGAGCTATCATCCCTGGGAAAGGAGGG | |
| | CACAGGGTTCAGAAGTAACTACTTTGCTTATGAAACTGGAGGCAA | |
| | GGCCTGCAAAATGCAATACTGCAAGCATTGGGGAGTCAGACTCCC | |
| | ATCAGGTGTCTGGTTCGAGATGGCTGATAAGGATCTCTTTGCTGCA | |
| | GCCAGATTCCCTGAATGCCCAGAAGGGTCAAGTATCTCTGCTCCAT | |
| | CTCAGACCTCAGTGGATGTAAGTCTAATTCAGGACGTTGAGAGGA | |
| | TCTTGGATTATTCCCTCTGCCAAGAAACCTGGAGCAAAATCAGAGC | |
| | GGGTCTTCCAATCTCTCCAGTGGATCTCAGCTATCTTGCTCCTAAA | |
| | AACCCAGGAACCGGTCCTGCTTTCACCATAATCAATGGTACCCTAA | |
| | AATACTTTGAGACCAGATACATCAGAGTCGATATTGCTGCTCCAAT | |
| | CCTCTCAAGAATGGTCGGAATGATCAGTGGAACTACCACAGAAAG | |
| | GGAACTGTGGGATGACTGGGCACCATATGAAGACGTGGAAATTGG | |
| | ACCCAATGGAGTTCTGAGGACCAGTTCAGGATATAAGTTTCCTTTA | |
| | TACATGATTGGACATGGTATGTTGGACTCCGATCTTCATCTTAGCT | |
| | CAAAGGCTCAGGTGTTCGAACATCCTCACATTCAAGACGCTGCTTC | |
| | GCAACTTCCTGATGATGAGAGTTTATTTTTTGGTGATACTGGGCTA | |
| | TCCAAAAATCCAATCGAGCTTGTAGAAGGTTGGTTCAGTAGTTGGA | |
| | AAAGCTCTATTGCCTCTTTTTTCTTTATCATAGGGTTAATCATTGGA | |
| | CTATTCTTGGTTCTCCGAGTTGGTATCCATCTTTGCATTAAATTAAA | |
| | GCACACCAAGAAAAGACAGATTTATACAGACATAGAGATGAACCG | |
| | ACTTGGAAAGATGGACGCCCAGACCAGGAGGAGGGAAAGGAGGG | |
| | CAGAGAAGCAGGCCCAATGGAAGGCCGCCAACTAAGGATCCAAG | |
| | CTTATCGATACCGTCGACCTCGAGGGCCCAGATCTAATTCACCCCA | |
| | CCAGTGCAGGCTGCCTATCAGAAAGTGGTGGCTGGTGTGGCTAAT | |
| | GCCCTGGCCCACAAGTATCACTAAGCTCGCTTTCTTGCTGTCCAAT | |
| | TTCTATTAAAGGTTCCTTTGTTCCCTAAGTCCAACTACTAAACTGG | |
| | GGGATATTATGAAGGGCCTTGAGCATCTGGATTCTGCCTAATAAAA | |
| | AACATTTATTTTCATTGCAATGATGTATTTAAATTATTTCTGAATAT | |
| | TTTACTAAAAAGGGAATGTGGGAGGTCAGTGCATTTAAAACATAA | |
| | AGAAATGAAGAGCTAGTTCAAACCTTGGGAAAATACACTATATCT | |
| | TAAACTCCATGAAAGAAGGTGAGGCTGCAAACAGCTAATGCACAT | |
| | TGGCAACAGCCCCTGATGCCTATGCCTTATTCATCCCTCAGAAAAG | |
| | GATTCAAGTAGAGGCTTGATTTGGAGGTTAAAGTTTTGCTATGCTG | |
| | TATTTTACATTACTTATTGTTTTAGCTGTCCTCATGAATGTCTTTTCA | |
| | CTACCCATTTGCTTATCCTGCATCTCTCAGCCTTGACTCCACTCAGT | |
| | TCTCTTGCTTAGAGATACCACCTTTCCCCTGAAGTGTTCCTTCCATG | |
| | TTTTACGGCGAGATGGTTTCTCCTCGCCTGGCCACTCAGCCTTAGTT | |
| | GTCTCTGTTGTCTTATAGAGGTCTACTTGAAGAAGGAAAAACAGG | |
| | GGGCATGGTTTGACTGTCCTGTGAGCCCTTCTTCCCTGCCTCCCCCA | |
| | CTCACAGTGACCCGGAATCCCTCGACATGGCAGTCTAGATCATTCT | |
| | TGAAGACGAAAGGGCCTCGTGATACGCCTATTTTTATAGGTTAATG | |
| | TCATGATAATAATGGTTTCTTAGACGTCAGGTGGCACTTTTCGGGG | |
| | AAATGTGCGCGGAACCCCTATTTGTTTATTTTTCTAAATACATTCA | |
| | AATATGTATCCGCTCATGAGACAATAACCCTGATAAATGCTTCAAT | |
| | AATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTATTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGAGATGCGCCGCGTGCGGCTGCTGGAGATGG | |
| | CGGACGCGATGGATATGTTCTGCCAAGGGTTGGTTTGCGCATTCAC | |
| | AGTTCTCCGCAAGAATTGATTGGCTCCAATTCTTGGAGTGGTGAAT | |
| | CCGTTAGCGAGGTGCCGCCGGCTTCCATTCAGGTCGAGGTGGCCCG | |
| | GCTCCATGCACCGCGACGCAACGCGGGGAGGCAGACAAGGTATAG | |
| | GGCGGCGCCTACAATCCATGCCAACCCGTTCCATGTGCTCGCCGAG | |
| | GCGGCATAAATCGCCGTGACGATCAGCGGTCCAATGATCGAAGTT | |
| | AGGCTGGTAAGAGCCGCGAGCGATCCTTGAAGCTGTCCCTGATGG | |
| | TCGTCATCTACCTGCCTGGACAGCATGGCCTGCAACGCGGGCATCC | |
| | CGATGCCGCCGGAAGCGAGAAGAATCATAATGGGGAAGGCCATCC | |
| | AGCCTCGCGTCGGGGAGCTTTTTGCAAAAGCCTAGGCCTCCAAAA | |
| | AAGCCTCCTCACTACTTCTGGAATAGCTCAGAGGCCGAGGCGGCCT | |
| | CGGCCTCTGCATAAATAAAAAAAATTAGTCAGCCATG | |
| 156 | AGCTTGGCCCATTGCATACGTTGTATCCATATCATAATATGTACAT | VSVG-mutλN |
| | TTATATTGGCTCATGTCCAACATTACCGCCATGTTGACATTGATTAT | |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGAAGTGCCTTT | |
| | TGTACTTAGCCTTTTTATTCATTGGGGTGAATTGCAAGTTCACCATA | |
| | GTTTTTCCACACAACCAAAAAGGAAACTGGAAAAATGTTCCTTCTA | |
| | ATTACCATTATTGCCCGTCAAGCTCAGATTTAAATTGGCATAATGA | |
| | CTTAATAGGCACAGCCTTACAAGTCAAAATGCCCAAGAGTCACAA | |
| | GGCTATTCAAGCAGACGGTTGGATGTGTCATGCTTCCAAATGGGTC | |
| | ACTACTTGTGATTTCCGCTGGTATGGACCGAAGTATATAACACATT | |
| | CCATCCGATCCTTCACTCCATCTGTAGAACAATGCAAGGAAAGCAT | |
| | TGAACAAACGAAACAAGGAACTTGGCTGAATCCAGGCTTCCCTCC | |
| | TCAAAGTTGTGGATATGCAACTGTGACGGATGCCGAAGCAGTGAT | |
| | TGTCCAGGTGACTCCTCACCATGTGCTGGTTGATGAATACACAGGA | |
| | GAATGGGTTGATTCACAGTTCATCAACGGAAAATGCAGCAATTAC | |
| | ATATGCCCCACTGTCCATAACTCTACAACCTGGCATTCTGACTATA | |
| | AGGTCAAAGGGCTATGTGATTCTAACCTCATTTCCATGGACATCAC | |
| | CTTCTTCTCAGAGGACGGAGAGCTATCATCCCTGGGAAAGGAGGG | |
| | CACAGGGTTCAGAAGTAACTACTTTGCTTATGAAACTGGAGGCAA | |
| | GGCCTGCAAAATGCAATACTGCAAGCATTGGGGAGTCAGACTCCC | |
| | ATCAGGTGTCTGGTTCGAGATGGCTGATAAGGATCTCTTTGCTGCA | |
| | GCCAGATTCCCTGAATGCCCAGAAGGGTCAAGTATCTCTGCTCCAT | |
| | CTCAGACCTCAGTGGATGTAAGTCTAATTCAGGACGTTGAGAGGA | |
| | TCTTGGATTATTCCCTCTGCCAAGAAACCTGGAGCAAAATCAGAGC | |
| | GGGTCTTCCAATCTCTCCAGTGGATCTCAGCTATCTTGCTCCTAAA | |
| | AACCCAGGAACCGGTCCTGCTTTCACCATAATCAATGGTACCCTAA | |
| | AATACTTTGAGACCAGATACATCAGAGTCGATATTGCTGCTCCAAT | |
| | CCTCTCAAGAATGGTCGGAATGATCAGTGGAACTACCACAGAAAG | |
| | GGAACTGTGGGATGACTGGGCACCATATGAAGACGTGGAAATTGG | |
| | ACCCAATGGAGTTCTGAGGACCAGTTCAGGATATAAGTTTCCTTTA | |
| | TACATGATTGGACATGGTATGTTGGACTCCGATCTTCATCTTAGCT | |
| | CAAAGGCTCAGGTGTTCGAACATCCTCACATTCAAGACGCTGCTTC | |
| | GCAACTTCCTGATGATGAGAGTTTATTTTTTGGTGATACTGGGCTA | |
| | TCCAAAAATCCAATCGAGCTTGTAGAAGGTTGGTTCAGTAGTTGGA | |
| | AAAGCTCTATTGCCTCTTTTTTCTTTATCATAGGGTTAATCATTGGA | |
| | CTATTCTTGGTTCTCCGAGTTGGTATCCATCTTTGCATTAAATTAAA | |
| | GCACACCAAGAAAAGACAGATTTATACAGACATAGAGATGAACCG | |
| | ACTTGGAAAGGGAAACGCCCGGACCAGGAGGAGGGAAAGGAGGG | |
| | CAGAGAAGCAGGCCCAATGGAAGGCCGCCAACTAAGGATCCAAG | |
| | CTTATCGATACCGTCGACCTCGAGGGCCCAGATCTAATTCACCCCA | |
| | CCAGTGCAGGCTGCCTATCAGAAAGTGGTGGCTGGTGTGGCTAAT | |
| | GCCCTGGCCCACAAGTATCACTAAGCTCGCTTTCTTGCTGTCCAAT | |
| | TTCTATTAAAGGTTCCTTTGTTCCCTAAGTCCAACTACTAAACTGG | |
| | GGGATATTATGAAGGGCCTTGAGCATCTGGATTCTGCCTAATAAAA | |
| | AACATTTATTTTCATTGCAATGATGTATTTAAATTATTTCTGAATAT | |
| | TTTACTAAAAAGGGAATGTGGGAGGTCAGTGCATTTAAAACATAA | |
| | AGAAATGAAGAGCTAGTTCAAACCTTGGGAAAATACACTATATCT | |
| | TAAACTCCATGAAAGAAGGTGAGGCTGCAAACAGCTAATGCACAT | |
| | TGGCAACAGCCCCTGATGCCTATGCCTTATTCATCCCTCAGAAAAG | |
| | GATTCAAGTAGAGGCTTGATTTGGAGGTTAAAGTTTTGCTATGCTG | |
| | TATTTTACATTACTTATTGTTTTAGCTGTCCTCATGAATGTCTTTTCA | |
| | CTACCCATTTGCTTATCCTGCATCTCTCAGCCTTGACTCCACTCAGT | |
| | TCTCTTGCTTAGAGATACCACCTTTCCCCTGAAGTGTTCCTTCCATG | |
| | TTTTACGGCGAGATGGTTTCTCCTCGCCTGGCCACTCAGCCTTAGTT | |
| | GTCTCTGTTGTCTTATAGAGGTCTACTTGAAGAAGGAAAAACAGG | |
| | GGGCATGGTTTGACTGTCCTGTGAGCCCTTCTTCCCTGCCTCCCCCA | |
| | CTCACAGTGACCCGGAATCCCTCGACATGGCAGTCTAGATCATTCT | |
| | TGAAGACGAAAGGGCCTCGTGATACGCCTATTTTTATAGGTTAATG | |
| | TCATGATAATAATGGTTTCTTAGACGTCAGGTGGCACTTTTCGGGG | |
| | AAATGTGCGCGGAACCCCTATTTGTTTATTTTTCTAAATACATTCA | |
| | AATATGTATCCGCTCATGAGACAATAACCCTGATAAATGCTTCAAT | |
| | AATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTATTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGAGATGCGCCGCGTGCGGCTGCTGGAGATGG | |
| | CGGACGCGATGGATATGTTCTGCCAAGGGTTGGTTTGCGCATTCAC | |
| | AGTTCTCCGCAAGAATTGATTGGCTCCAATTCTTGGAGTGGTGAAT | |
| | CCGTTAGCGAGGTGCCGCCGGCTTCCATTCAGGTCGAGGTGGCCCG | |
| | GCTCCATGCACCGCGACGCAACGCGGGGAGGCAGACAAGGTATAG | |
| | GGCGGCGCCTACAATCCATGCCAACCCGTTCCATGTGCTCGCCGAG | |
| | GCGGCATAAATCGCCGTGACGATCAGCGGTCCAATGATCGAAGTT | |
| | AGGCTGGTAAGAGCCGCGAGCGATCCTTGAAGCTGTCCCTGATGG | |
| | TCGTCATCTACCTGCCTGGACAGCATGGCCTGCAACGCGGGCATCC | |
| | CGATGCCGCCGGAAGCGAGAAGAATCATAATGGGGAAGGCCATCC | |
| | AGCCTCGCGTCGGGGAGCTTTTTGCAAAAGCCTAGGCCTCCAAAA | |
| | AAGCCTCCTCACTACTTCTGGAATAGCTCAGAGGCCGAGGCGGCCT | |
| | CGGCCTCTGCATAAATAAAAAAAATTAGTCAGCCATG | |
| 157 | MKCLLYLAFLFIGVNCKFTIVFPHNQKGNWKNVPSNYHYCPSSSDLN | VSVG-MS2_{cp} |
| | WHNDLIGTALQVKMPKSHKAIQADGWMCHASKWVTTCDFRWYGPK | aa |
| | YITHSIRSFTPSVEQCKESIEQTKQGTWLNPGFPPQSCGYATVTDAEAV | |
| | IVQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKMASNFTQFVLVDNGGT | |
| | GDVTVAPSNFANGIAEWISSNSRSQAYKVTCSVRQSSAQNRKYTIKVE | |
| | VPKGAWRSYLNMELTIPIFATNSDCELIVKAMQGLLKDGNPIPSAIAA | |
| | NSGIY | |
| 158 | MKCLLYLAFLFIGVNCKFTIVFPHNQKGNWKNVPSNYHYCPSSSDLN | VSVG-td- |
| | WHNDLIGTALQVKMPKSHKAIQADGWMCHASKWVTTCDFRWYGPK | MS2_{cp} |
| | YITHSIRSFTPSVEQCKESIEQTKQGTWLNPGFPPQSCGYATVIDAEAV | |
| | IVQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKMASNFTQFVLVDNGGT | |
| | GDVTVAPSNFANGIAEWISSNSRSQAYKVTCSVRQSSAQNRKYTIKVE | |
| | VPKGAWRSYLNMELTIPIFATNSDCELIVKAMQGLLKDGNPIPSAIAA | |
| | NSGIYAMASNFTQFVLVDNGGTGDVTVAPSNFANGIAEWISSNSRSQ | |
| | AYKVTCSVRQSSAQNRKYTIKVEVPKGAWRSYLNMELTIPIFATNSDC | |
| | ELIVKAMQGLLKDGNPIPSAIAANSGIY | |
| 159 | MGARASVLSGGELDKWEKIRLRPGGKKQYKLKHIVWASRELERFAV | MA (N-term)- |
| | NPGLLETSEGCRQILGQLQPSLQTGSEELRSLYNTIAVLYCVHQRIDVK | λN |
| | DTKEALDKIEEEQNKSKKKAQQAAADTGNNSQVSQNYPIVQMDAQT | |
| | RRRERRAEKQAQWKAAN | |
| 160 | MGARASVLSGGELDKWEKIRLRPGGKKQYKLKHIVWASRELERFAV | MA (N-term)- |
| | NPGLLETSEGCRQILGQLQPSLQTGSEELRSLYNTIAVLYCVHQRIDVK | mutλN |
| | DTKEALDKIEEEQNKSKKKAQQAAADTGNNSQVSQNYPIVQGNART | |
| | RRRERRAEKQAQWKAAN | |
| 161 | MKCLLYLAFLFIGVNCKFTIVFPHNQKGNWKNVPSNYHYCPSSSDLN | VSVG-λN |
| | WHNDLIGTALQVKMPKSHKAIQADGWMCHASKWVTTCDFRWYGPK | |
| | YITHSIRSFTPSVEQCKESIEQTKQGTWLNPGFPPQSCGYATVTDAEAV | |
| | IVQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKMDAQTRRRERRAEKQA | |
| | QWKAAN | |
| 162 | MKCLLYLAFLFIGVNCKFTIVFPHNQKGNWKNVPSNYHYCPSSSDLN | VSVG-mutλN |
| | WHNDLIGTALQVKMPKSHKAIQADGWMCHASKWVTTCDFRWYGPK | |
| | YITHSIRSFTPSVEQCKESIEQTKQGTWLNPGFPPQSCGYATVTDAEAV | |
| | IVQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKGNARTRRRERRAEKQA | |
| | QWKAAN | |
| 163 | AGCTTGGCCCATTGCATACGTTGTATCCATATCATAATATGTACAT | 1x MS2 with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCTAAGGTACCTAATTGCCTAGAAAACATGAGGATCACCC | |
| | ATGTCTGCAGGTCGACTCTAGAAAGTCGACCTCGAGGGCCCAGAT | |
| | CTAATTCACCCCACCAGTGCAGGCTGCCTATCAGAAAGTGGTGGCT | |
| | GGTGTGGCTAATGCCCTGGCCCACAAGTATCACTAAGCTCGCTTTC | |
| | TTGCTGTCCAATTTCTATTAAAGGTTCCTTTGTTCCCTAAGTCCAAC | |
| | TACTAAACTGGGGGATATTATGAAGGGCCTTGAGCATCTGGATTCT | |
| | GCCTAATAAAAAACATTTATTTTCATTGCAATGATGTATTTAAATT | |
| | ATTTCTGAATATTTTACTAAAAAGGGAATGTGGGAGGTCAGTGCAT | |
| | TTAAAACATAAAGAAATGAAGAGCTAGTTCAAACCTTGGGAAAAT | |
| | ACACTATATCTTAAACTCCATGAAAGAAGGTGAGGCTGCAAACAG | |
| | CTAATGCACATTGGCAACAGCCCCTGATGCCTATGCCTTATTCATC | |
| | CCTCAGAAAAGGATTCAAGTAGAGGCTTGATTTGGAGGTTAAAGT | |
| | TTTGCTATGCTGTATTTTACATTACTTATTGTTTTAGCTGTCCTCATG | |
| | AATGTCTTTTCACTACCCATTTGCTTATCCTGCATCTCTCAGCCTTG | |
| | ACTCCACTCAGTTCTCTTGCTTAGAGATACCACCTTTCCCCTGAAGT | |
| | GTTCCTTCCATGTTTTACGGCGAGATGGTTTCTCCTCGCCTGGCCAC | |
| | TCAGCCTTAGTTGTCTCTGTTGTCTTATAGAGGTCTACTTGAAGAA | |
| | GGAAAAACAGGGGGCATGGTTTGACTGTCCTGTGAGCCCTTCTTCC | |
| | CTGCCTCCCCCACTCACAGTGACCCGGAATCCCTCGACATGGCAGT | |
| | CTAGATCATTCTTGAAGACGAAAGGGCCTCGTGATACGCCTATTTT | |
| | TATAGGTTAATGTCATGATAATAATGGTTTCTTAGACGTCAGGTGG | |
| | CACTTTTCGGGGAAATGTGCGCGGAACCCCTATTTGTTTATTTTTCT | |
| | AAATACATTCAAATATGTATCCGCTCATGAGACAATAACCCTGATA | |
| | AATGCTTCAATAATATTGAAAAAGGAAGAGTATGAGTATTCAACA | |
| | TTTCCGTGTCGCCCTTATTCCCTTTTTTGCGGCATTTTGCCTTCCTGT | |
| | TTTTGCTCACCCAGAAACGCTGGTGAAAGTAAAAGATGCTGAAGA | |
| | TCAGTTGGGTGCACGAGTGGGTTACATCGAACTGGATCTCAACAG | |
| | CGGTAAGATCCTTGAGAGTTTTCGCCCCGAAGAACGTTTTCCAATG | |
| | ATGAGCACTTTTAAAGTTCTGCTATGTGGCGCGGTATTATCCCGTA | |
| | TTGACGCCGGGCAAGAGCAACTCGGTCGCCGCATACACTATTCTCA | |
| | GAATGACTTGGTTGAGTACTCACCAGTCACAGAAAAGCATCTTAC | |
| | GGATGGCATGACAGTAAGAGAATTATGCAGTGCTGCCATAACCAT | |
| | GAGTGATAACACTGCGGCCAACTTACTTCTGACAACGATCGGAGG | |
| | ACCGAAGGAGCTAACCGCTTTTTTGCACAACATGGGGGATCATGT | |
| | AACTCGCCTTGATCGTTGGGAACCGGAGCTGAATGAAGCCATACC | |
| | AAACGACGAGCGTGACACCACGATGCCTGTAGCAATGGCAACAAC | |
| | GTTGCGCAAACTATTAACTGGCGAACTACTTACTCTAGCTTCCCGG | |
| | CAACAATTAATAGACTGGATGGAGGCGGATAAAGTTGCAGGACCA | |
| | CTTCTGCGCTCGGCCCTTCCGGCTGGCTGGTTTATTGCTGATAAATC | |
| | TGGAGCCGGTGAGCGTGGGTCTCGCGGTATCATTGCAGCACTGGG | |
| | GCCAGATGGTAAGCCCTCCCGTATCGTAGTTATCTACACGACGGGG | |
| | AGTCAGGCAACTATGGATGAACGAAATAGACAGATCGCTGAGATA | |
| | GGTGCCTCACTGATTAAGCATTGGTAACTGTCAGACCAAGTTTACT | |
| | CATATATACTTTAGATTGATTTAAAACTTCATTTTTAATTTAAAAGG | |
| | ATCTAGGTGAAGATCCTTTTTGATAATCTCATGACCAAAATCCCTT | |
| | AACGTGAGTTTTCGTTCCACTGAGCGTCAGACCCCGTAGAAAAGAT | |
| | CAAAGGATCTTCTTGAGATCCTTTTTTTCTGCGCGTAATCTGCTGCT | |
| | TGCAAACAAAAAAACCACCGCTACCAGCGGTGGTTTGTTTGCCGG | |
| | ATCAAGAGCTACCAACTCTTTTTCCGAAGGTAACTGGCTTCAGCAG | |
| | AGCGCAGATACCAAATACTGTTCTTCTAGTGTAGCCGTAGTTAGGC | |
| | CACCACTTCAAGAACTCTGTAGCACCGCCTACATACCTCGCTCTGC | |
| | TAATCCTGTTACCAGTGGCTGCTGCCAGTGGCGATAAGTCGTGTCT | |
| | TACCGGGTTGGACTCAAGACGATAGTTACCGGATAAGGCGCAGCG | |
| | GTCGGGCTGAACGGGGGGTTCGTGCACACAGCCCAGCTTGGAGCG | |
| | AACGACCTACACCGAACTGAGATACCTACAGCGTGAGCTATGAGA | |
| | AAGCGCCACGCTTCCCGAAGGGAGAAAGGCGGACAGGTATCCGGT | |
| | AAGCGGCAGGGTCGGAACAGGAGAGCGCACGAGGGAGCTTCCAG | |
| | GGGGAAACGCCTGGTATCTTTATAGTCCTGTCGGGTTTCGCCACCT | |
| | CTGACTTGAGCGTCGATTTTTGTGATGCTCGTCAGGGGGGCGGAGC | |
| | CTATGGAAAAACGCCAGCAACGGATGCGCCGCGTGCGGCTGCTGG | |
| | AGATGGCGGACGCGATGGATATGTTCTGCCAAGGGTTGGTTTGCG | |
| | CATTCACAGTTCTCCGCAAGAATTGATTGGCTCCAATTCTTGGAGT | |
| | GGTGAATCCGTTAGCGAGGTGCCGCCGGCTTCCATTCAGGTCGAG | |
| | GTGGCCCGGCTCCATGCACCGCGACGCAACGCGGGGAGGCAGACA | |
| | AGGTATAGGGCGGCGCCTACAATCCATGCCAACCCGTTCCATGTGC | |
| | TCGCCGAGGCGGCATAAATCCCCGTGACGATCAGCGGTCCAATGA | |
| | TCGAAGTTAGGCTGGTAAGAGCCGCGAGCGATCCTTGAAGCTGTC | |
| | CCTGATGGTCGTCATCTACCTGCCTGGACAGCATGGCCTGCAACGC | |
| | GGGCATCCCGATGCCGCCGGAAGCGAGAAGAATCATAATGGGGAA | |
| | GGCCATCCAGCCTCGCGTCGGGGAGCTTTTTGCAAAAGCCTAGGCC | |
| | TCCAAAAAAGCCTCCTCACTACTTCTGGAATAGCTCAGAGGCCGA | |
| | GGCGGCCTCGGCCTCTGCATAAATAAAAAAAATTAGTCAGCCATG | |
| 164 | AGCTTGGCCCATTGCATACGTTGTATCCATATCATAATATGTACAT | 2x MS2 with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCTAAGGTACCTAATTGCCTAGAAAACATGAGGATCACCC | |
| | ATGTCTGCAGGTCGACTCTAGAAAACATGAGGATCACCCATGTCTG | |
| | CAGTATTCCCGGGTTCATTAGATCCGTCGACCTCGAGGGCCCAGAT | |
| | CTAATTCACCCCACCAGTGCAGGCTGCCTATCAGAAAGTGGTGGCT | |
| | GGTGTGGCTAATGCCCTGGCCCACAAGTATCACTAAGCTCGCTTTC | |
| | TTGCTGTCCAATTTCTATTAAAGGTTCCTTTGTTCCCTAAGTCCAAC | |
| | TACTAAACTGGGGGATATTATGAAGGGCCTTGAGCATCTGGATTCT | |
| | GCCTAATAAAAAACATTTATTTTCATTGCAATGATGTATTTAAATT | |
| | ATTTCTGAATATTTTACTAAAAAGGGAATGTGGGAGGTCAGTGCAT | |
| | TTAAAACATAAAGAAATGAAGAGCTAGTTCAAACCTTGGGAAAAT | |
| | ACACTATATCTTAAACTCCATGAAAGAAGGTGAGGCTGCAAACAG | |
| | CTAATGCACATTGGCAACAGCCCCTGATGCCTATGCCTTATTCATC | |
| | CCTCAGAAAAGGATTCAAGTAGAGGCTTGATTTGGAGGTTAAAGT | |
| | TTTGCTATGCTGTATTTTACATTACTTATTGTTTTAGCTGTCCTCATG | |
| | AATGTCTTTTCACTACCCATTTGCTTATCCTGCATCTCTCAGCCTTG | |
| | ACTCCACTCAGTTCTCTTGCTTAGAGATACCACCTTTCCCCTGAAGT | |
| | GTTCCTTCCATGTTTTACGGCGAGATGGTTTCTCCTCGCCTGGCCAC | |
| | TCAGCCTTAGTTGTCTCTGTTGTCTTATAGAGGTCTACTTGAAGAA | |
| | GGAAAAACAGGGGGCATGGTTTGACTGTCCTGTGAGCCCTTCTTCC | |
| | CTGCCTCCCCCACTCACAGTGACCCGGAATCCCTCGACATGGCAGT | |
| | CTAGATCATTCTTGAAGACGAAAGGGCCTCGTGATACGCCTATTTT | |
| | TATAGGTTAATGTCATGATAATAATGGTTTCTTAGACGTCAGGTGG | |
| | CACTTTTCGGGGAAATGTGCGCGGAACCCCTATTTGTTTATTTTTCT | |
| | AAATACATTCAAATATGTATCCGCTCATGAGACAATAACCCTGATA | |
| | AATGCTTCAATAATATTGAAAAAGGAAGAGTATGAGTATTCAACA | |
| | TTTCCGTGTCGCCCTTATTCCCTTTTTTGCGGCATTTTGCCTTCCTGT | |
| | TTTTGCTCACCCAGAAACGCTGGTGAAAGTAAAAGATGCTGAAGA | |
| | TCAGTTGGGTGCACGAGTGGGTTACATCGAACTGGATCTCAACAG | |
| | CGGTAAGATCCTTGAGAGTTTTCGCCCCGAAGAACGTTTTCCAATG | |
| | ATGAGCACTTTTAAAGTTCTGCTATGTGGCGCGGTATTATCCCGTA | |
| | TTGACGCCGGGCAAGAGCAACTCGGTCGCCGCATACACTATTCTCA | |
| | GAATGACTTGGTTGAGTACTCACCAGTCACAGAAAAGCATCTTAC | |
| | GGATGGCATGACAGTAAGAGAATTATGCAGTGCTGCCATAACCAT | |
| | GAGTGATAACACTGCGGCCAACTTACTTCTGACAACGATCGGAGG | |
| | ACCGAAGGAGCTAACCGCTTTTTTGCACAACATGGGGGATCATGT | |
| | AACTCGCCTTGATCGTTGGGAACCGGAGCTGAATGAAGCCATACC | |
| | AAACGACGAGCGTGACACCACGATGCCTGTAGCAATGGCAACAAC | |
| | GTTGCGCAAACTATTAACTGGCGAACTACTTACTCTAGCTTCCCGG | |
| | CAACAATTAATAGACTGGATGGAGGCGGATAAAGTTGCAGGACCA | |
| | CTTCTGCGCTCGGCCCTTCCGGCTGGCTGGTTTATTGCTGATAAATC | |
| | TGGAGCCGGTGAGCGTGGGTCTCGCGGTATCATTGCAGCACTGGG | |
| | GCCAGATGGTAAGCCCTCCCGTATCGTAGTTATCTACACGACGGGG | |
| | AGTCAGGCAACTATGGATGAACGAAATAGACAGATCGCTGAGATA | |
| | GGTGCCTCACTGATTAAGCATTGGTAACTGTCAGACCAAGTTTACT | |
| | CATATATACTTTAGATTGATTTAAAACTTCATTTTTAATTTAAAAGG | |
| | ATCTAGGTGAAGATCCTTTTTGATAATCTCATGACCAAAATCCCTT | |
| | AACGTGAGTTTTCGTTCCACTGAGCGTCAGACCCCGTAGAAAAGAT | |
| | CAAAGGATCTTCTTGAGATCCTTTTTTTCTGCGCGTAATCTGCTGCT | |
| | TGCAAACAAAAAAACCACCGCTACCAGCGGTGGTTTGTTTGCCGG | |
| | ATCAAGAGCTACCAACTCTTTTTCCGAAGGTAACTGGCTTCAGCAG | |
| | AGCGCAGATACCAAATACTGTTCTTCTAGTGTAGCCGTAGTTAGGC | |
| | CACCACTTCAAGAACTCTGTAGCACCGCCTACATACCTCGCTCTGC | |
| | TAATCCTGTTACCAGTGGCTGCTGCCAGTGGCGATAAGTCGTGTCT | |
| | TACCGGGTTGGACTCAAGACGATAGTTACCGGATAAGGCGCAGCG | |
| | GTCGGGCTGAACGGGGGGTTCGTGCACACAGCCCAGCTTGGAGCG | |
| | AACGACCTACACCGAACTGAGATACCTACAGCGTGAGCTATGAGA | |
| | AAGCGCCACGCTTCCCGAAGGGAGAAAGGCGGACAGGTATCCGGT | |
| | AAGCGGCAGGGTCGGAACAGGAGAGCGCACGAGGGAGCTTCCAG | |
| | GGGGAAACGCCTGGTATCTTTATAGTCCTGTCGGGTTTCGCCACCT | |
| | CTGACTTGAGCGTCGATTTTTGTGATGCTCGTCAGGGGGGCGGAGC | |
| | CTATGGAAAAACGCCAGCAACGGATGCGCCGCGTGCGGCTGCTGG | |
| | AGATGGCGGACGCGATGGATATGTTCTGCCAAGGGTTGGTTTGCG | |
| | CATTCACAGTTCTCCGCAAGAATTGATTGGCTCCAATTCTTGGAGT | |
| | GGTGAATCCGTTAGCGAGGTGCCGCCGGCTTCCATTCAGGTCGAG | |
| | GTGGCCCGGCTCCATGCACCGCGACGCAACGCGGGGAGGCAGACA | |
| | AGGTATAGGGCGGCGCCTACAATCCATGCCAACCCGTTCCATGTGC | |
| | TCGCCGAGGCGGCATAAATCCCCGTGACGATCAGCGGTCCAATGA | |
| | TCGAAGTTAGGCTGGTAAGAGCCGCGAGCGATCCTTGAAGCTGTC | |
| | CCTGATGGTCGTCATCTACCTGCCTGGACAGCATGGCCTGCAACGC | |
| | GGGCATCCCGATGCCGCCGGAAGCGAGAAGAATCATAATGGGGAA | |
| | GGCCATCCAGCCTCGCGTCGGGGAGCTTTTTGCAAAAGCCTAGGCC | |
| | TCCAAAAAAGCCTCCTCACTACTTCTGGAATAGCTCAGAGGCCGA | |
| | GGCGGCCTCGGCCTCTGCATAAATAAAAAAAATTAGTCAGCCATG | |
| | AGCTTGGCCCATTGCATACGTTGTATCCATATCATAATATGTACAT | |
| | TTATATTGGCTCATGTCCAACATTACCGCCATGTTGACATTGATTAT | |
| | TGACTAGTTATTAATAGTAATCAATTACGGGGTCATTAGTTCATAG | |
| 165 | CCCATATATGGAGTTCCGCGTTACATAACTTACGGTAAATGGCCCG | 6x MS2 with |
| | CCTGGCTGACCGCCCAACGACCCCCGCCCATTGACGTCAATAATGA | Cre |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAACCTACAAACGGGTGGAGGATCACCCCACCCGACACT | |
| | TCACAATCAAGGGGTACAATACACAAGGGTGGAGGAACACCCCAC | |
| | CCTCCAGACACATTACACAGAAATCCAATCAAACAGAAGCACCAT | |
| | CAGGGCTTCTGCTACCAAATTTATCTCAAAAAACTACAACAAGGA | |
| | ATCACCATCAGGGATTCCCTGTGCAATATACGTCAAACGAGGGCC | |
| | ACGACGGGAGGACGATCACGCCTCCCGAATATCGGCATGTCTGGC | |
| | TTTCGAATTCAGTGCGTGGAGCATCAGCCCACGCAGCCAATCAGA | |
| | GTCGAATACAAGTCGACCTCGAGGGCCCAGATCTAATTCACCCCA | |
| | CCAGTGCAGGCTGCCTATCAGAAAGTGGTGGCTGGTGTGGCTAAT | |
| | GCCCTGGCCCACAAGTATCACTAAGCTCGCTTTCTTGCTGTCCAAT | |
| | TTCTATTAAAGGTTCCTTTGTTCCCTAAGTCCAACTACTAAACTGG | |
| | GGGATATTATGAAGGGCCTTGAGCATCTGGATTCTGCCTAATAAAA | |
| | AACATTTATTTTCATTGCAATGATGTATTTAAATTATTTCTGAATAT | |
| | TTTACTAAAAAGGGAATGTGGGAGGTCAGTGCATTTAAAACATAA | |
| | AGAAATGAAGAGCTAGTTCAAACCTTGGGAAAATACACTATATCT | |
| | TAAACTCCATGAAAGAAGGTGAGGCTGCAAACAGCTAATGCACAT | |
| | TGGCAACAGCCCCTGATGCCTATGCCTTATTCATCCCTCAGAAAAG | |
| | GATTCAAGTAGAGGCTTGATTTGGAGGTTAAAGTTTTGCTATGCTG | |
| | TATTTTACATTACTTATTGTTTTAGCTGTCCTCATGAATGTCTTTTCA | |
| | CTACCCATTTGCTTATCCTGCATCTCTCAGCCTTGACTCCACTCAGT | |
| | TCTCTTGCTTAGAGATACCACCTTTCCCCTGAAGTGTTCCTTCCATG | |
| | TTTTACGGCGAGATGGTTTCTCCTCGCCTGGCCACTCAGCCTTAGTT | |
| | GTCTCTGTTGTCTTATAGAGGTCTACTTGAAGAAGGAAAAACAGG | |
| | GGGCATGGTTTGACTGTCCTGTGAGCCCTTCTTCCCTGCCTCCCCCA | |
| | CTCACAGTGACCCGGAATCCCTCGACATGGCAGTCTAGATCATTCT | |
| | TGAAGACGAAAGGGCCTCGTGATACGCCTATTTTTATAGGTTAATG | |
| | TCATGATAATAATGGTTTCTTAGACGTCAGGTGGCACTTTTCGGGG | |
| | AAATGTGCGCGGAACCCCTATTTGTTTATTTTTCTAAATACATTCA | |
| | AATATGTATCCGCTCATGAGACAATAACCCTGATAAATGCTTCAAT | |
| | AATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTATTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGATGCGCCGCGTGCGGCTGCTGGAGATGGCG | |
| | GACGCGATGGATATGTTCTGCCAAGGGTTGGTTTGCGCATTCACAG | |
| | TTCTCCGCAAGAATTGATTGGCTCCAATTCTTGGAGTGGTGAATCC | |
| | GTTAGCGAGGTGCCGCCGGCTTCCATTCAGGTCGAGGTGGCCCGG | |
| | CTCCATGCACCGCGACGCAACGCGGGGAGGCAGACAAGGTATAGG | |
| | GCGGCGCCTACAATCCATGCCAACCCGTTCCATGTGCTCGCCGAGG | |
| | CGGCATAAATCCCCGTGACGATCAGCGGTCCAATGATCGAAGTTA | |
| | GGCTGGTAAGAGCCGCGAGCGATCCTTGAAGCTGTCCCTGATGGT | |
| | CGTCATCTACCTGCCTGGACAGCATGGCCTGCAACGCGGGCATCCC | |
| | GATGCCGCCGGAAGCGAGAAGAATCATAATGGGGAAGGCCATCCA | |
| | GCCTCGCGTCGGGGAGCTTTTTGCAAAAGCCTAGGCCTCCAAAAA | |
| | AGCCTCCTCACTACTTCTGGAATAGCTCAGAGGCCGAGGCGGCCTC | |
| | GGCCTCTGCATAAATAAAAAAAATTAGTCAGCCATG | |
| 166 | AGCTTGGCCCATTGCATACGTTGTATCCATATCATAATATGTACAT | 12x MS2 with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAACCTACAAACGGGTGGAGGATCACCCCACCCGACACT | |
| | TCACAATCAAGGGGTACAATACACAAGGGTGGAGGAACACCCCAC | |
| | CCTCCAGACACATTACACAGAAATCCAATCAAACAGAAGCACCAT | |
| | CAGGGCTTCTGCTACCAAATTTATCTCAAAAAACTACAACAAGGA | |
| | ATCACCATCAGGGATTCCCTGTGCAATATACGTCAAACGAGGGCC | |
| | ACGACGGGAGGACGATCACGCCTCCCGAATATCGGCATGTCTGGC | |
| | TTTCGAATTCAGTGCGTGGAGCATCAGCCCACGCAGCCAATCAGA | |
| | GTCGAATACAAGTCGACTTTCGCGAAGAGCATCAGCCTTCGCGCC | |
| | ATTCTTACACAAACCACACTCTCCCCTACAGGAACAGCATCAGCGT | |
| | TCCTGCCCAGTACCCAACTCAAGAAAATTTATGTCCCCATGCAGCA | |
| | TCAGCGCATGGGCCCCAAGAATACATCCCCAACAAAATCACATCC | |
| | GAGCACCAACAGGGCTCGGAGTGTTGTTTCTTGTCCAACTGGACAA | |
| | ACCCTCCATGGACCATCAGGCCATGGACTCTCACCAACAAGACAA | |
| | AAACTACTCTTCTCGAAGCAGCATCAGCGCTTCGAAACACTCGACC | |
| | TCGAGGGCCCAGATCTAATTCACCCCACCAGTGCAGGCTGCCTATC | |
| | AGAAAGTGGTGGCTGGTGTGGCTAATGCCCTGGCCCACAAGTATC | |
| | ACTAAGCTCGCTTTCTTGCTGTCCAATTTCTATTAAAGGTTCCTTTG | |
| | TTCCCTAAGTCCAACTACTAAACTGGGGGATATTATGAAGGGCCTT | |
| | GAGCATCTGGATTCTGCCTAATAAAAAACATTTATTTTCATTGCAA | |
| | TGATGTATTTAAATTATTTCTGAATATTTTACTAAAAAGGGAATGT | |
| | GGGAGGTCAGTGCATTTAAAACATAAAGAAATGAAGAGCTAGTTC | |
| | AAACCTTGGGAAAATACACTATATCTTAAACTCCATGAAAGAAGG | |
| | TGAGGCTGCAAACAGCTAATGCACATTGGCAACAGCCCCTGATGC | |
| | CTATGCCTTATTCATCCCTCAGAAAAGGATTCAAGTAGAGGCTTGA | |
| | TTTGGAGGTTAAAGTTTTGCTATGCTGTATTTTACATTACTTATTGT | |
| | TTTAGCTGTCCTCATGAATGTCTTTTCACTACCCATTTGCTTATCCT | |
| | GCATCTCTCAGCCTTGACTCCACTCAGTTCTCTTGCTTAGAGATACC | |
| | ACCTTTCCCCTGAAGTGTTCCTTCCATGTTTTACGGCGAGATGGTTT | |
| | CTCCTCGCCTGGCCACTCAGCCTTAGTTGTCTCTGTTGTCTTATAGA | |
| | GGTCTACTTGAAGAAGGAAAAACAGGGGGCATGGTTTGACTGTCC | |
| | TGTGAGCCCTTCTTCCCTGCCTCCCCCACTCACAGTGACCCGGAAT | |
| | CCCTCGACATGGCAGTCTAGATCATTCTTGAAGACGAAAGGGCCTC | |
| | GTGATACGCCTATTTTTATAGGTTAATGTCATGATAATAATGGTTTC | |
| | TTAGACGTCAGGTGGCACTTTTCGGGGAAATGTGCGCGGAACCCCT | |
| | ATTTGTTTATTTTTCTAAATACATTCAAATATGTATCCGCTCATGAG | |
| | ACAATAACCCTGATAAATGCTTCAATAATATTGAAAAAGGAAGAG | |
| | TATGAGTATTCAACATTTCCGTGTCGCCCTTATTCCCTTTTTTGCGG | |
| | CATTTTGCCTTCCTGTTTTTGCTCACCCAGAAACGCTGGTGAAAGT | |
| | AAAAGATGCTGAAGATCAGTTGGGTGCACGAGTGGGTTACATCGA | |
| | ACTGGATCTCAACAGCGGTAAGATCCTTGAGAGTTTTCGCCCCGAA | |
| | GAACGTTTTCCAATGATGAGCACTTTTAAAGTTCTGCTATGTGGCG | |
| | CGGTATTATCCCGTATTGACGCCGGGCAAGAGCAACTCGGTCGCC | |
| | GCATACACTATTCTCAGAATGACTTGGTTGAGTACTCACCAGTCAC | |
| | AGAAAAGCATCTTACGGATGGCATGACAGTAAGAGAATTATGCAG | |
| | TGCTGCCATAACCATGAGTGATAACACTGCGGCCAACTTACTTCTG | |
| | ACAACGATCGGAGGACCGAAGGAGCTAACCGCTTTTTTGCACAAC | |
| | ATGGGGGATCATGTAACTCGCCTTGATCGTTGGGAACCGGAGCTG | |
| | AATGAAGCCATACCAAACGACGAGCGTGACACCACGATGCCTGTA | |
| | GCAATGGCAACAACGTTGCGCAAACTATTAACTGGCGAACTACTT | |
| | ACTCTAGCTTCCCGGCAACAATTAATAGACTGGATGGAGGCGGAT | |
| | AAAGTTGCAGGACCACTTCTGCGCTCGGCCCTTCCGGCTGGCTGGT | |
| | TTATTGCTGATAAATCTGGAGCCGGTGAGCGTGGGTCTCGCGGTAT | |
| | CATTGCAGCACTGGGGCCAGATGGTAAGCCCTCCCGTATCGTAGTT | |
| | ATCTACACGACGGGGAGTCAGGCAACTATGGATGAACGAAATAGA | |
| | CAGATCGCTGAGATAGGTGCCTCACTGATTAAGCATTGGTAACTGT | |
| | CAGACCAAGTTTACTCATATATACTTTAGATTGATTTAAAACTTCA | |
| | TTTTTAATTTAAAAGGATCTAGGTGAAGATCCTTTTTGATAATCTCA | |
| | TGACCAAAATCCCTTAACGTGAGTTTTCGTTCCACTGAGCGTCAGA | |
| | CCCCGTAGAAAAGATCAAAGGATCTTCTTGAGATCCTTTTTTTCTG | |
| | CGCGTAATCTGCTGCTTGCAAACAAAAAAACCACCGCTACCAGCG | |
| | GTGGTTTGTTTGCCGGATCAAGAGCTACCAACTCTTTTTCCGAAGG | |
| | TAACTGGCTTCAGCAGAGCGCAGATACCAAATACTGTTCTTCTAGT | |
| | GTAGCCGTAGTTAGGCCACCACTTCAAGAACTCTGTAGCACCGCCT | |
| | ACATACCTCGCTCTGCTAATCCTGTTACCAGTGGCTGCTGCCAGTG | |
| | GCGATAAGTCGTGTCTTACCGGGTTGGACTCAAGACGATAGTTACC | |
| | GGATAAGGCGCAGCGGTCGGGCTGAACGGGGGGTTCGTGCACACA | |
| | GCCCAGCTTGGAGCGAACGACCTACACCGAACTGAGATACCTACA | |
| | GCGTGAGCTATGAGAAAGCGCCACGCTTCCCGAAGGGAGAAAGGC | |
| | GGACAGGTATCCGGTAAGCGGCAGGGTCGGAACAGGAGAGCGCA | |
| | CGAGGGAGCTTCCAGGGGGAAACGCCTGGTATCTTTATAGTCCTGT | |
| | CGGGTTTCGCCACCTCTGACTTGAGCGTCGATTTTTGTGATGCTCGT | |
| | CAGGGGGGCGGAGCCTATGGAAAAACGCCAGCAACGGATGCGCC | |
| | GCGTGCGGCTGCTGGAGATGGCGGACGCGATGGATATGTTCTGCC | |
| | AAGGGTTGGTTTGCGCATTCACAGTTCTCCGCAAGAATTGATTGGC | |
| | TCCAATTCTTGGAGTGGTGAATCCGTTAGCGAGGTGCCGCCGGCTT | |
| | CCATTCAGGTCGAGGTGGCCCGGCTCCATGCACCGCGACGCAACG | |
| | CGGGGAGGCAGACAAGGTATAGGGCGGCGCCTACAATCCATGCCA | |
| | ACCCGTTCCATGTGCTCGCCGAGGCGGCATAAATCCCCGTGACGAT | |
| | CAGCGGTCCAATGATCGAAGTTAGGCTGGTAAGAGCCGCGAGCGA | |
| | TCCTTGAAGCTGTCCCTGATGGTCGTCATCTACCTGCCTGGACAGC | |
| | ATGGCCTGCAACGCGGGCATCCCGATGCCGCCGGAAGCGAGAAGA | |
| | ATCATAATGGGGAAGGCCATCCAGCCTCGCGTCGGGGAGCTTTTTG | |
| | CAAAAGCCTAGGCCTCCAAAAAAGCCTCCTCACTACTTCTGGAATA | |
| | GCTCAGAGGCCGAGGCGGCCTCGGCCTCTGCATAAATAAAAAAAA | |
| | TTAGTCAGCCATG | |
| | AGCTTGGCCCATTGCATACGTTGTATCCATATCATAATATGTACAT | 24x MS2 with |
| 167 | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAACCTACAAACGGGTGGAGGATCACCCCACCCGACACT | |
| | TCACAATCAAGGGGTACAATACACAAGGGTGGAGGAACACCCCAC | |
| | CCTCCAGACACATTACACAGAAATCCAATCAAACAGAAGCACCAT | |
| | CAGGGCTTCTGCTACCAAATTTATCTCAAAAAACTACAACAAGGA | |
| | ATCACCATCAGGGATTCCCTGTGCAATATACGTCAAACGAGGGCC | |
| | ACGACGGGAGGACGATCACGCCTCCCGAATATCGGCATGTCTGGC | |
| | TTTCGAATTCAGTGCGTGGAGCATCAGCCCACGCAGCCAATCAGA | |
| | GTCGAATACAAGTCGACTTTCGCGAAGAGCATCAGCCTTCGCGCC | |
| | ATTCTTACACAAACCACACTCTCCCCTACAGGAACAGCATCAGCGT | |
| | TCCTGCCCAGTACCCAACTCAAGAAAATTTATGTCCCCATGCAGCA | |
| | TCAGCGCATGGGCCCCAAGAATACATCCCCAACAAAATCACATCC | |
| | GAGCACCAACAGGGCTCGGAGTGTTGTTTCTTGTCCAACTGGACAA | |
| | ACCCTCCATGGACCATCAGGCCATGGACTCTCACCAACAAGACAA | |
| | AAACTACTCTTCTCGAAGCAGCATCAGCGCTTCGAAACACTCGAGC | |
| | ATACATTGTGCCTATTTCTTGGGTGGACGATCACGCCACCCATGCT | |
| | CTCACGAATTTCAAAACACGGACAAGGACGAGCACCACCAGGGCT | |
| | CGTCGTTCCACGTCCAATACGATTACTTACCTTTCGGGATCACGAT | |
| | CACGGATCCCGCAGCTACATCACTTCCACTCAGGACATTCAAGCAT | |
| | GCACGATCACGGCATGCTCCACAAGTCTCAACCACAGAAACTACC | |
| | AAATGGGTTCAGCACCAGCGAACCCACTCCTACCTCAAACCTCTTC | |
| | CCACAAAACTGGCAAGCAGGATCACCGCTTGCCCATTCCAACATA | |
| | CCAAATCAAAAACAATTACTGGTACAGCATCAGCGTACCAGCCCA | |
| | CATCTCTCACTACTATCAAAAACCAAACCGTTCAGCAACAGCGAA | |
| | CGGTACACACGGAAAAATCAACTGGTTTACAAATACGAAAGACGA | |
| | TCACGCTTTCGTCCAGCGCAAACTATTACGAAAAACATCCGACGG | |
| | GAAGAGCAACAGCCTTCCCGCGGCGGAAAACCTCACAAAAACACG | |
| | ACAAACGGATGCACGAACACGGCATCCGCCGACAACCCACAAACT | |
| | TACAACCAGGCAAACGGTGCAGGATCACCGCACCGTACATCAAAC | |
| | ACCTCAGATCTCATGTCGACCTCGAGGGCCCAGATCTAATTCACCC | |
| | CACCAGTGCAGGCTGCCTATCAGAAAGTGGTGGCTGGTGTGGCTA | |
| | ATGCCCTGGCCCACAAGTATCACTAAGCTCGCTTTCTTGCTGTCCA | |
| | ATTTCTATTAAAGGTTCCTTTGTTCCCTAAGTCCAACTACTAAACTG | |
| | GGGGATATTATGAAGGGCCTTGAGCATCTGGATTCTGCCTAATAAA | |
| | AAACATTTATTTTCATTGCAATGATGTATTTAAATTATTTCTGAATA | |
| | TTTTACTAAAAAGGGAATGTGGGAGGTCAGTGCATTTAAAACATA | |
| | AAGAAATGAAGAGCTAGTTCAAACCTTGGGAAAATACACTATATC | |
| | TTAAACTCCATGAAAGAAGGTGAGGCTGCAAACAGCTAATGCACA | |
| | TTGGCAACAGCCCCTGATGCCTATGCCTTATTCATCCCTCAGAAAA | |
| | GGATTCAAGTAGAGGCTTGATTTGGAGGTTAAAGTTTTGCTATGCT | |
| | GTATTTTACATTACTTATTGTTTTAGCTGTCCTCATGAATGTCTTTTC | |
| | ACTACCCATTTGCTTATCCTGCATCTCTCAGCCTTGACTCCACTCAG | |
| | TTCTCTTGCTTAGAGATACCACCTTTCCCCTGAAGTGTTCCTTCCAT | |
| | GTTTTACGGCGAGATGGTTTCTCCTCGCCTGGCCACTCAGCCTTAG | |
| | TTGTCTCTGTTGTCTTATAGAGGTCTACTTGAAGAAGGAAAAACAG | |
| | GGGGCATGGTTTGACTGTCCTGTGAGCCCTTCTTCCCTGCCTCCCCC | |
| | ACTCACAGTGACCCGGAATCCCTCGACATGGCAGTCTAGATCATTC | |
| | TTGAAGACGAAAGGGCCTCGTGATACGCCTATTTTTATAGGTTAAT | |
| | GTCATGATAATAATGGTTTCTTAGACGTCAGGTGGCACTTTTCGGG | |
| | GAAATGTGCGCGGAACCCCTATTTGTTTATTTTTCTAAATACATTC | |
| | AAATATGTATCCGCTCATGAGACAATAACCCTGATAAATGCTTCAA | |
| | TAATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTATTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGATGCGCCGCGTGCGGCTGCTGGAGATGGCG | |
| | GACGCGATGGATATGTTCTGCCAAGGGTTGGTTTGCGCATTCACAG | |
| | TTCTCCGCAAGAATTGATTGGCTCCAATTCTTGGAGTGGTGAATCC | |
| | GTTAGCGAGGTGCCGCCGGCTTCCATTCAGGTCGAGGTGGCCCGG | |
| | CTCCATGCACCGCGACGCAACGCGGGGAGGCAGACAAGGTATAGG | |
| | GCGGCGCCTACAATCCATGCCAACCCGTTCCATGTGCTCGCCGAGG | |
| | CGGCATAAATCCCCGTGACGATCAGCGGTCCAATGATCGAAGTTA | |
| | GGCTGGTAAGAGCCGCGAGCGATCCTTGAAGCTGTCCCTGATGGT | |
| | CGTCATCTACCTGCCTGGACAGCATGGCCTGCAACGCGGGCATCCC | |
| | GATGCCGCCGGAAGCGAGAAGAATCATAATGGGGAAGGCCATCCA | |
| | GCCTCGCGTCGGGGAGCTTTTTGCAAAAGCCTAGGCCTCCAAAAA | |
| | AGCCTCCTCACTACTTCTGGAATAGCTCAGAGGCCGAGGCGGCCTC | |
| | GGCCTCTGCATAAATAAAAAAAATTAGTCAGCCATG | |
| 168 | AGCTTGGCCCATTGCATACGTTGTATCCATATCATAATATGTACAT | 1x boxB with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAAGTCCAACTACTAAACTGGGGATTCCTGGGCCCTGAA | |
| | GAAGGGCCCCTCGTCGACCTCGAGGGCCCAGATCTAATTCACCCC | |
| | ACCAGTGCAGGCTGCCTATCAGAAAGTGGTGGCTGGTGTGGCTAA | |
| | TGCCCTGGCCCACAAGTATCACTAAGCTCGCTTTCTTGCTGTCCAA | |
| | TTTCTATTAAAGGTTCCTTTGTTCCCTAAGTCCAACTACTAAACTGG | |
| | GGGATATTATGAAGGGCCTTGAGCATCTGGATTCTGCCTAATAAAA | |
| | AACATTTATTTTCATTGCAATGATGTATTTAAATTATTTCTGAATAT | |
| | TTTACTAAAAAGGGAATGTGGGAGGTCAGTGCATTTAAAACATAA | |
| | AGAAATGAAGAGCTAGTTCAAACCTTGGGAAAATACACTATATCT | |
| | TAAACTCCATGAAAGAAGGTGAGGCTGCAAACAGCTAATGCACAT | |
| | TGGCAACAGCCCCTGATGCCTATGCCTTATTCATCCCTCAGAAAAG | |
| | GATTCAAGTAGAGGCTTGATTTGGAGGTTAAAGTTTTGCTATGCTG | |
| | TATTTTACATTACTTATTGTTTTAGCTGTCCTCATGAATGTCTTTTCA | |
| | CTACCCATTTGCTTATCCTGCATCTCTCAGCCTTGACTCCACTCAGT | |
| | TCTCTTGCTTAGAGATACCACCTTTCCCCTGAAGTGTTCCTTCCATG | |
| | TTTTACGGCGAGATGGTTTCTCCTCGCCTGGCCACTCAGCCTTAGTT | |
| | GTCTCTGTTGTCTTATAGAGGTCTACTTGAAGAAGGAAAAACAGG | |
| | GGGCATGGTTTGACTGTCCTGTGAGCCCTTCTTCCCTGCCTCCCCCA | |
| | CTCACAGTGACCCGGAATCCCTCGACATGGCAGTCTAGATCATTCT | |
| | TGAAGACGAAAGGGCCTCGTGATACGCCTATTTTTATAGGTTAATG | |
| | TCATGATAATAATGGTTTCTTAGACGTCAGGTGGCACTTTTCGGGG | |
| | AAATGTGCGCGGAACCCCTATTTGTTTATTTTTCTAAATACATTCA | |
| | AATATGTATCCGCTCATGAGACAATAACCCTGATAAATGCTTCAAT | |
| | AATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTATTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGATGCGCCGCGTGCGGCTGCTGGAGATGGCG | |
| | GACGCGATGGATATGTTCTGCCAAGGGTTGGTTTGCGCATTCACAG | |
| | TTCTCCGCAAGAATTGATTGGCTCCAATTCTTGGAGTGGTGAATCC | |
| | GTTAGCGAGGTGCCGCCGGCTTCCATTCAGGTCGAGGTGGCCCGG | |
| | CTCCATGCACCGCGACGCAACGCGGGGAGGCAGACAAGGTATAGG | |
| | GCGGCGCCTACAATCCATGCCAACCCGTTCCATGTGCTCGCCGAGG | |
| | CGGCATAAATCCCCGTGACGATCAGCGGTCCAATGATCGAAGTTA | |
| | GGCTGGTAAGAGCCGCGAGCGATCCTTGAAGCTGTCCCTGATGGT | |
| | CGTCATCTACCTGCCTGGACAGCATGGCCTGCAACGCGGGCATCCC | |
| | GATGCCGCCGGAAGCGAGAAGAATCATAATGGGGAAGGCCATCCA | |
| | GCCTCGCGTCGGGGAGCTTTTTGCAAAAGCCTAGGCCTCCAAAAA | |
| | AGCCTCCTCACTACTTCTGGAATAGCTCAGAGGCCGAGGCGGCCTC | |
| | GGCCTCTGCATAAATAAAAAAAATTAGTCAGCCATG | |
| 169 | AGCTTGGCCCATTGCATACGTTGTATCCATATCATAATATGTACAT | 2x boxB with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAAGTCCAACTACTAAACTGGGGATTCCTGGGCCCTGAA | |
| | GAAGGGCCCCTCGACTAAGTCCAACTACTAAACTGGGCCCTGAAG | |
| | AAGGGCCCATATAGTCGACCTCGAGGGCCCAGATCTAATTCACCC | |
| | CACCAGTGCAGGCTGCCTATCAGAAAGTGGTGGCTGGTGTGGCTA | |
| | ATGCCCTGGCCCACAAGTATCACTAAGCTCGCTTTCTTGCTGTCCA | |
| | ATTTCTATTAAAGGTTCCTTTGTTCCCTAAGTCCAACTACTAAACTG | |
| | GGGGATATTATGAAGGGCCTTGAGCATCTGGATTCTGCCTAATAAA | |
| | AAACATTTATTTTCATTGCAATGATGTATTTAAATTATTTCTGAATA | |
| | TTTTACTAAAAAGGGAATGTGGGAGGTCAGTGCATTTAAAACATA | |
| | AAGAAATGAAGAGCTAGTTCAAACCTTGGGAAAATACACTATATC | |
| | TTAAACTCCATGAAAGAAGGTGAGGCTGCAAACAGCTAATGCACA | |
| | TTGGCAACAGCCCCTGATGCCTATGCCTTATTCATCCCTCAGAAAA | |
| | GGATTCAAGTAGAGGCTTGATTTGGAGGTTAAAGTTTTGCTATGCT | |
| | GTATTTTACATTACTTATTGTTTTAGCTGTCCTCATGAATGTCTTTTC | |
| | ACTACCCATTTGCTTATCCTGCATCTCTCAGCCTTGACTCCACTCAG | |
| | TTCTCTTGCTTAGAGATACCACCTTTCCCCTGAAGTGTTCCTTCCAT | |
| | GTTTTACGGCGAGATGGTTTCTCCTCGCCTGGCCACTCAGCCTTAG | |
| | TTGTCTCTGTTGTCTTATAGAGGTCTACTTGAAGAAGGAAAAACAG | |
| | GGGGCATGGTTTGACTGTCCTGTGAGCCCTTCTTCCCTGCCTCCCCC | |
| | ACTCACAGTGACCCGGAATCCCTCGACATGGCAGTCTAGATCATTC | |
| | TTGAAGACGAAAGGGCCTCGTGATACGCCTATTTTTATAGGTTAAT | |
| | GTCATGATAATAATGGTTTCTTAGACGTCAGGTGGCACTTTTCGGG | |
| | GAAATGTGCGCGGAACCCCTATTTGTTTATTTTTCTAAATACATTC | |
| | AAATATGTATCCGCTCATGAGACAATAACCCTGATAAATGCTTCAA | |
| | TAATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTATTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGATGCGCCGCGTGCGGCTGCTGGAGATGGCG | |
| | GACGCGATGGATATGTTCTGCCAAGGGTTGGTTTGCGCATTCACAG | |
| | TTCTCCGCAAGAATTGATTGGCTCCAATTCTTGGAGTGGTGAATCC | |
| | GTTAGCGAGGTGCCGCCGGCTTCCATTCAGGTCGAGGTGGCCCGG | |
| | CTCCATGCACCGCGACGCAACGCGGGGAGGCAGACAAGGTATAGG | |
| | GCGGCGCCTACAATCCATGCCAACCCGTTCCATGTGCTCGCCGAGG | |
| | CGGCATAAATCCCCGTGACGATCAGCGGTCCAATGATCGAAGTTA | |
| | GGCTGGTAAGAGCCGCGAGCGATCCTTGAAGCTGTCCCTGATGGT | |
| | CGTCATCTACCTGCCTGGACAGCATGGCCTGCAACGCGGGCATCCC | |
| | GATGCCGCCGGAAGCGAGAAGAATCATAATGGGGAAGGCCATCCA | |
| | GCCTCGCGTCGGGGAGCTTTTTGCAAAAGCCTAGGCCTCCAAAAA | |
| | AGCCTCCTCACTACTTCTGGAATAGCTCAGAGGCCGAGGCGGCCTC | |
| | GGCCTCTGCATAAATAAAAAAAATTAGTCAGCCATG | |
| 170 | AGCTTGGCCCATTGCATACGTTGTATCCATATCATAATATGTACAT | 5x boxB with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAAGTCCAACTACTAAACTGGGGATTCCTGGGCCCTGAA | |
| | GAAGGGCCCCTCGACTAAGTCCAACTACTAAACTGGGCCCTGAAG | |
| | AAGGGCCCATATAGGGCCCTGAAGAAGGGCCCTATCGAGGATATT | |
| | ATCTCGACTAAGTCCAACTACTAAACTGGGCCCTGAAGAAGGGCC | |
| | CATATAGGGCCCTGAAGAAGGGCCCTATCGAGGATATTATCAATC | |
| | TTCCTGCTCAGTGTCGACCTCGAGGGCCCAGATCTAATTCACCCCA | |
| | CCAGTGCAGGCTGCCTATCAGAAAGTGGTGGCTGGTGTGGCTAAT | |
| | GCCCTGGCCCACAAGTATCACTAAGCTCGCTTTCTTGCTGTCCAAT | |
| | TTCTATTAAAGGTTCCTTTGTTCCCTAAGTCCAACTACTAAACTGG | |
| | GGGATATTATGAAGGGCCTTGAGCATCTGGATTCTGCCTAATAAAA | |
| | AACATTTATTTTCATTGCAATGATGTATTTAAATTATTTCTGAATAT | |
| | TTTACTAAAAAGGGAATGTGGGAGGTCAGTGCATTTAAAACATAA | |
| | AGAAATGAAGAGCTAGTTCAAACCTTGGGAAAATACACTATATCT | |
| | TAAACTCCATGAAAGAAGGTGAGGCTGCAAACAGCTAATGCACAT | |
| | TGGCAACAGCCCCTGATGCCTATGCCTTATTCATCCCTCAGAAAAG | |
| | GATTCAAGTAGAGGCTTGATTTGGAGGTTAAAGTTTTGCTATGCTG | |
| | TATTTTACATTACTTATTGTTTTAGCTGTCCTCATGAATGTCTTTTCA | |
| | CTACCCATTTGCTTATCCTGCATCTCTCAGCCTTGACTCCACTCAGT | |
| | TCTCTTGCTTAGAGATACCACCTTTCCCCTGAAGTGTTCCTTCCATG | |
| | TTTTACGGCGAGATGGTTTCTCCTCGCCTGGCCACTCAGCCTTAGTT | |
| | GTCTCTGTTGTCTTATAGAGGTCTACTTGAAGAAGGAAAAACAGG | |
| | GGGCATGGTTTGACTGTCCTGTGAGCCCTTCTTCCCTGCCTCCCCCA | |
| | CTCACAGTGACCCGGAATCCCTCGACATGGCAGTCTAGATCATTCT | |
| | TGAAGACGAAAGGGCCTCGTGATACGCCTATTTTTATAGGTTAATG | |
| | TCATGATAATAATGGTTTCTTAGACGTCAGGTGGCACTTTTCGGGG | |
| | AAATGTGCGCGGAACCCCTATTTGTTTATTTTTCTAAATACATTCA | |
| | AATATGTATCCGCTCATGAGACAATAACCCTGATAAATGCTTCAAT | |
| | AATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTATTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGATGCGCCGCGTGCGGCTGCTGGAGATGGCG | |
| | GACGCGATGGATATGTTCTGCCAAGGGTTGGTTTGCGCATTCACAG | |
| | TTCTCCGCAAGAATTGATTGGCTCCAATTCTTGGAGTGGTGAATCC | |
| | GTTAGCGAGGTGCCGCCGGCTTCCATTCAGGTCGAGGTGGCCCGG | |
| | CTCCATGCACCGCGACGCAACGCGGGGAGGCAGACAAGGTATAGG | |
| | GCGGCGCCTACAATCCATGCCAACCCGTTCCATGTGCTCGCCGAGG | |
| | CGGCATAAATCCCCGTGACGATCAGCGGTCCAATGATCGAAGTTA | |
| | GGCTGGTAAGAGCCGCGAGCGATCCTTGAAGCTGTCCCTGATGGT | |
| | CGTCATCTACCTGCCTGGACAGCATGGCCTGCAACGCGGGCATCCC | |
| | GATGCCGCCGGAAGCGAGAAGAATCATAATGGGGAAGGCCATCCA | |
| | GCCTCGCGTCGGGGAGCTTTTTGCAAAAGCCTAGGCCTCCAAAAA | |
| | AGCCTCCTCACTACTTCTGGAATAGCTCAGAGGCCGAGGCGGCCTC | |
| | GGCCTCTGCATAAATAAAAAAAATTAGTCAGCCATG | |
| 171 | AGCTTGGCCCATTGCATACGTTGTATCCATATCATAATATGTACAT | 10x boxB with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAAGTCCAACTACTAAACTGGGGATTCCTGGGCCCTGAA | |
| | GAAGGGCCCCTCGACTAAGTCCAACTACTAAACTGGGCCCTGAAG | |
| | AAGGGCCCATATAGGGCCCTGAAGAAGGGCCCTATCGAGGATATT | |
| | ATCTCGACTAAGTCCAACTACTAAACTGGGCCCTGAAGAAGGGCC | |
| | CATATAGGGCCCTGAAGAAGGGCCCTATCGAGGATATTATCAATC | |
| | TTCCTGCTCAGTAAGCAAGTCCAACTACTAAACTGGGGATTCCTGG | |
| | GCCCTGAAGAAGGGCCCCTCGACTAAGTCCAACTACTAAACTGGG | |
| | CCCTGAAGAAGGGCCCATATAGGGCCCTGAAGAAGGGCCCTATCG | |
| | AGGATATTATCTCGACTAAGTCCAACTACTAAACTGGGCCCTGAAG | |
| | AAGGGCCCATATAGGGCCCTGAAGAAGGGCCCTATCGAGGATATT | |
| | ATCAATCTTCCTGCTCAGTGTCGACCTCGAGGGCCCAGATCTAATT | |
| | CACCCCACCAGTGCAGGCTGCCTATCAGAAAGTGGTGGCTGGTGT | |
| | GGCTAATGCCCTGGCCCACAAGTATCACTAAGCTCGCTTTCTTGCT | |
| | GTCCAATTTCTATTAAAGGTTCCTTTGTTCCCTAAGTCCAACTACTA | |
| | AACTGGGGGATATTATGAAGGGCCTTGAGCATCTGGATTCTGCCTA | |
| | ATAAAAAACATTTATTTTCATTGCAATGATGTATTTAAATTATTTCT | |
| | GAATATTTTACTAAAAAGGGAATGTGGGAGGTCAGTGCATTTAAA | |
| | ACATAAAGAAATGAAGAGCTAGTTCAAACCTTGGGAAAATACACT | |
| | ATATCTTAAACTCCATGAAAGAAGGTGAGGCTGCAAACAGCTAAT | |
| | GCACATTGGCAACAGCCCCTGATGCCTATGCCTTATTCATCCCTCA | |
| | GAAAAGGATTCAAGTAGAGGCTTGATTTGGAGGTTAAAGTTTTGCT | |
| | ATGCTGTATTTTACATTACTTATTGTTTTAGCTGTCCTCATGAATGT | |
| | CTTTTCACTACCCATTTGCTTATCCTGCATCTCTCAGCCTTGACTCC | |
| | ACTCAGTTCTCTTGCTTAGAGATACCACCTTTCCCCTGAAGTGTTCC | |
| | TTCCATGTTTTACGGCGAGATGGTTTCTCCTCGCCTGGCCACTCAG | |
| | CCTTAGTTGTCTCTGTTGTCTTATAGAGGTCTACTTGAAGAAGGAA | |
| | AAACAGGGGGCATGGTTTGACTGTCCTGTGAGCCCTTCTTCCCTGC | |
| | CTCCCCCACTCACAGTGACCCGGAATCCCTCGACATGGCAGTCTAG | |
| | ATCATTCTTGAAGACGAAAGGGCCTCGTGATACGCCTATTTTTATA | |
| | GGTTAATGTCATGATAATAATGGTTTCTTAGACGTCAGGTGGCACT | |
| | TTTCGGGGAAATGTGCGCGGAACCCCTATTTGTTTATTTTTCTAAAT | |
| | ACATTCAAATATGTATCCGCTCATGAGACAATAACCCTGATAAATG | |
| | CTTCAATAATATTGAAAAAGGAAGAGTATGAGTATTCAACATTTCC | |
| | GTGTCGCCCTTATTCCCTTTTTTGCGGCATTTTGCCTTCCTGTTTTTG | |
| | CTCACCCAGAAACGCTGGTGAAAGTAAAAGATGCTGAAGATCAGT | |
| | TGGGTGCACGAGTGGGTTACATCGAACTGGATCTCAACAGCGGTA | |
| | AGATCCTTGAGAGTTTTCGCCCCGAAGAACGTTTTCCAATGATGAG | |
| | CACTTTTAAAGTTCTGCTATGTGGCGCGGTATTATCCCGTATTGAC | |
| | GCCGGGCAAGAGCAACTCGGTCGCCGCATACACTATTCTCAGAAT | |
| | GACTTGGTTGAGTACTCACCAGTCACAGAAAAGCATCTTACGGAT | |
| | GGCATGACAGTAAGAGAATTATGCAGTGCTGCCATAACCATGAGT | |
| | GATAACACTGCGGCCAACTTACTTCTGACAACGATCGGAGGACCG | |
| | AAGGAGCTAACCGCTTTTTTGCACAACATGGGGGATCATGTAACTC | |
| | GCCTTGATCGTTGGGAACCGGAGCTGAATGAAGCCATACCAAACG | |
| | ACGAGCGTGACACCACGATGCCTGTAGCAATGGCAACAACGTTGC | |
| | GCAAACTATTAACTGGCGAACTACTTACTCTAGCTTCCCGGCAACA | |
| | ATTAATAGACTGGATGGAGGCGGATAAAGTTGCAGGACCACTTCT | |
| | GCGCTCGGCCCTTCCGGCTGGCTGGTTTATTGCTGATAAATCTGGA | |
| | GCCGGTGAGCGTGGGTCTCGCGGTATCATTGCAGCACTGGGGCCA | |
| | GATGGTAAGCCCTCCCGTATCGTAGTTATCTACACGACGGGGAGTC | |
| | AGGCAACTATGGATGAACGAAATAGACAGATCGCTGAGATAGGTG | |
| | CCTCACTGATTAAGCATTGGTAACTGTCAGACCAAGTTTACTCATA | |
| | TATACTTTAGATTGATTTAAAACTTCATTTTTAATTTAAAAGGATCT | |
| | AGGTGAAGATCCTTTTTGATAATCTCATGACCAAAATCCCTTAACG | |
| | TGAGTTTTCGTTCCACTGAGCGTCAGACCCCGTAGAAAAGATCAAA | |
| | GGATCTTCTTGAGATCCTTTTTTTCTGCGCGTAATCTGCTGCTTGCA | |
| | AACAAAAAAACCACCGCTACCAGCGGTGGTTTGTTTGCCGGATCA | |
| | AGAGCTACCAACTCTTTTTCCGAAGGTAACTGGCTTCAGCAGAGCG | |
| | CAGATACCAAATACTGTTCTTCTAGTGTAGCCGTAGTTAGGCCACC | |
| | ACTTCAAGAACTCTGTAGCACCGCCTACATACCTCGCTCTGCTAAT | |
| | CCTGTTACCAGTGGCTGCTGCCAGTGGCGATAAGTCGTGTCTTACC | |
| | GGGTTGGACTCAAGACGATAGTTACCGGATAAGGCGCAGCGGTCG | |
| | GGCTGAACGGGGGGTTCGTGCACACAGCCCAGCTTGGAGCGAACG | |
| | ACCTACACCGAACTGAGATACCTACAGCGTGAGCTATGAGAAAGC | |
| | GCCACGCTTCCCGAAGGGAGAAAGGCGGACAGGTATCCGGTAAGC | |
| | GGCAGGGTCGGAACAGGAGAGCGCACGAGGGAGCTTCCAGGGGG | |
| | AAACGCCTGGTATCTTTATAGTCCTGTCGGGTTTCGCCACCTCTGA | |
| | CTTGAGCGTCGATTTTTGTGATGCTCGTCAGGGGGGCGGAGCCTAT | |
| | GGAAAAACGCCAGCAACGGATGCGCCGCGTGCGGCTGCTGGAGAT | |
| | GGCGGACGCGATGGATATGTTCTGCCAAGGGTTGGTTTGCGCATTC | |
| | ACAGTTCTCCGCAAGAATTGATTGGCTCCAATTCTTGGAGTGGTGA | |
| | ATCCGTTAGCGAGGTGCCGCCGGCTTCCATTCAGGTCGAGGTGGCC | |
| | CGGCTCCATGCACCGCGACGCAACGCGGGGAGGCAGACAAGGTAT | |
| | AGGGCGGCGCCTACAATCCATGCCAACCCGTTCCATGTGCTCGCCG | |
| | AGGCGGCATAAATCCCCGTGACGATCAGCGGTCCAATGATCGAAG | |
| | TTAGGCTGGTAAGAGCCGCGAGCGATCCTTGAAGCTGTCCCTGATG | |
| | GTCGTCATCTACCTGCCTGGACAGCATGGCCTGCAACGCGGGCATC | |
| | CCGATGCCGCCGGAAGCGAGAAGAATCATAATGGGGAAGGCCATC | |
| | CAGCCTCGCGTCGGGGAGCTTTTTGCAAAAGCCTAGGCCTCCAAA | |
| | AAAGCCTCCTCACTACTTCTGGAATAGCTCAGAGGCCGAGGCGGC | |
| | CTCGGCCTCTGCATAAATAAAAAAAATTAGTCAGCCATG | |
| 172 | AGCTTGGCCCATTGCATACGTTGTATCCATATCATAATATGTACAT | 15x boxB with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAAGTCCAACTACTAAACTGGGGATTCCTGGGCCCTGAA | |
| | GAAGGGCCCCTCGACTAAGTCCAACTACTAAACTGGGCCCTGAAG | |
| | AAGGGCCCATATAGGGCCCTGAAGAAGGGCCCTATCGAGGATATT | |
| | ATCTCGACTAAGTCCAACTACTAAACTGGGCCCTGAAGAAGGGCC | |
| | CATATAGGGCCCTGAAGAAGGGCCCTATCGAGGATATTATCAATC | |
| | TTCCTGCTCAGTAAGCAAGTCCAACTACTAAACTGGGGATTCCTGG | |
| | GCCCTGAAGAAGGGCCCCTCGACTAAGTCCAACTACTAAACTGGG | |
| | CCCTGAAGAAGGGCCCATATAGGGCCCTGAAGAAGGGCCCTATCG | |
| | AGGATATTATCTCGACTAAGTCCAACTACTAAACTGGGCCCTGAAG | |
| | AAGGGCCCATATAGGGCCCTGAAGAAGGGCCCTATCGAGGATATT | |
| | ATCAATCTTCCTGCTCAGTGAAAAAGTCCAACTACTAAACTGGGGA | |
| | TTCCTGGGCCCTGAAGAAGGGCCCCTCGACTAAGTCCAACTACTAA | |
| | ACTGGGCCCTGAAGAAGGGCCCATATAGGGCCCTGAAGAAGGGCC | |
| | CTATCGAGGATATTATCTCGACTAAGTCCAACTACTAAACTGGGCC | |
| | CTGAAGAAGGGCCCATATAGGGCCCTGAAGAAGGGCCCTATCGAG | |
| | GATATTATCAATCTTCCTGCTCAGTGTCGACCTCGAGGGCCCAGAT | |
| | CTAATTCACCCCACCAGTGCAGGCTGCCTATCAGAAAGTGGTGGCT | |
| | GGTGTGGCTAATGCCCTGGCCCACAAGTATCACTAAGCTCGCTTTC | |
| | TTGCTGTCCAATTTCTATTAAAGGTTCCTTTGTTCCCTAAGTCCAAC | |
| | TACTAAACTGGGGGATATTATGAAGGGCCTTGAGCATCTGGATTCT | |
| | GCCTAATAAAAAACATTTATTTTCATTGCAATGATGTATTTAAATT | |
| | ATTTCTGAATATTTTACTAAAAAGGGAATGTGGGAGGTCAGTGCAT | |
| | TTAAAACATAAAGAAATGAAGAGCTAGTTCAAACCTTGGGAAAAT | |
| | ACACTATATCTTAAACTCCATGAAAGAAGGTGAGGCTGCAAACAG | |
| | CTAATGCACATTGGCAACAGCCCCTGATGCCTATGCCTTATTCATC | |
| | CCTCAGAAAAGGATTCAAGTAGAGGCTTGATTTGGAGGTTAAAGT | |
| | TTTGCTATGCTGTATTTTACATTACTTATTGTTTTAGCTGTCCTCATG | |
| | AATGTCTTTTCACTACCCATTTGCTTATCCTGCATCTCTCAGCCTTG | |
| | ACTCCACTCAGTTCTCTTGCTTAGAGATACCACCTTTCCCCTGAAGT | |
| | GTTCCTTCCATGTTTTACGGCGAGATGGTTTCTCCTCGCCTGGCCAC | |
| | TCAGCCTTAGTTGTCTCTGTTGTCTTATAGAGGTCTACTTGAAGAA | |
| | GGAAAAACAGGGGGCATGGTTTGACTGTCCTGTGAGCCCTTCTTCC | |
| | CTGCCTCCCCCACTCACAGTGACCCGGAATCCCTCGACATGGCAGT | |
| | CTAGATCATTCTTGAAGACGAAAGGGCCTCGTGATACGCCTATTTT | |
| | TATAGGTTAATGTCATGATAATAATGGTTTCTTAGACGTCAGGTGG | |
| | CACTTTTCGGGGAAATGTGCGCGGAACCCCTATTTGTTTATTTTTCT | |
| | AAATACATTCAAATATGTATCCGCTCATGAGACAATAACCCTGATA | |
| | AATGCTTCAATAATATTGAAAAAGGAAGAGTATGAGTATTCAACA | |
| | TTTCCGTGTCGCCCTTATTCCCTTTTTTGCGGCATTTTGCCTTCCTGT | |
| | TTTTGCTCACCCAGAAACGCTGGTGAAAGTAAAAGATGCTGAAGA | |
| | TCAGTTGGGTGCACGAGTGGGTTACATCGAACTGGATCTCAACAG | |
| | CGGTAAGATCCTTGAGAGTTTTCGCCCCGAAGAACGTTTTCCAATG | |
| | ATGAGCACTTTTAAAGTTCTGCTATGTGGCGCGGTATTATCCCGTA | |
| | TTGACGCCGGGCAAGAGCAACTCGGTCGCCGCATACACTATTCTCA | |
| | GAATGACTTGGTTGAGTACTCACCAGTCACAGAAAAGCATCTTAC | |
| | GGATGGCATGACAGTAAGAGAATTATGCAGTGCTGCCATAACCAT | |
| | GAGTGATAACACTGCGGCCAACTTACTTCTGACAACGATCGGAGG | |
| | ACCGAAGGAGCTAACCGCTTTTTTGCACAACATGGGGGATCATGT | |
| | AACTCGCCTTGATCGTTGGGAACCGGAGCTGAATGAAGCCATACC | |
| | AAACGACGAGCGTGACACCACGATGCCTGTAGCAATGGCAACAAC | |
| | GTTGCGCAAACTATTAACTGGCGAACTACTTACTCTAGCTTCCCGG | |
| | CAACAATTAATAGACTGGATGGAGGCGGATAAAGTTGCAGGACCA | |
| | CTTCTGCGCTCGGCCCTTCCGGCTGGCTGGTTTATTGCTGATAAATC | |
| | TGGAGCCGGTGAGCGTGGGTCTCGCGGTATCATTGCAGCACTGGG | |
| | GCCAGATGGTAAGCCCTCCCGTATCGTAGTTATCTACACGACGGGG | |
| | AGTCAGGCAACTATGGATGAACGAAATAGACAGATCGCTGAGATA | |
| | GGTGCCTCACTGATTAAGCATTGGTAACTGTCAGACCAAGTTTACT | |
| | CATATATACTTTAGATTGATTTAAAACTTCATTTTTAATTTAAAAGG | |
| | ATCTAGGTGAAGATCCTTTTTGATAATCTCATGACCAAAATCCCTT | |
| | AACGTGAGTTTTCGTTCCACTGAGCGTCAGACCCCGTAGAAAAGAT | |
| | CAAAGGATCTTCTTGAGATCCTTTTTTTCTGCGCGTAATCTGCTGCT | |
| | TGCAAACAAAAAAACCACCGCTACCAGCGGTGGTTTGTTTGCCGG | |
| | ATCAAGAGCTACCAACTCTTTTTCCGAAGGTAACTGGCTTCAGCAG | |
| | AGCGCAGATACCAAATACTGTTCTTCTAGTGTAGCCGTAGTTAGGC | |
| | CACCACTTCAAGAACTCTGTAGCACCGCCTACATACCTCGCTCTGC | |
| | TAATCCTGTTACCAGTGGCTGCTGCCAGTGGCGATAAGTCGTGTCT | |
| | TACCGGGTTGGACTCAAGACGATAGTTACCGGATAAGGCGCAGCG | |
| | GTCGGGCTGAACGGGGGGTTCGTGCACACAGCCCAGCTTGGAGCG | |
| | AACGACCTACACCGAACTGAGATACCTACAGCGTGAGCTATGAGA | |
| | AAGCGCCACGCTTCCCGAAGGGAGAAAGGCGGACAGGTATCCGGT | |
| | AAGCGGCAGGGTCGGAACAGGAGAGCGCACGAGGGAGCTTCCAG | |
| | GGGGAAACGCCTGGTATCTTTATAGTCCTGTCGGGTTTCGCCACCT | |
| | CTGACTTGAGCGTCGATTTTTGTGATGCTCGTCAGGGGGGCGGAGC | |
| | CTATGGAAAAACGCCAGCAACGGATGCGCCGCGTGCGGCTGCTGG | |
| | AGATGGCGGACGCGATGGATATGTTCTGCCAAGGGTTGGTTTGCG | |
| | CATTCACAGTTCTCCGCAAGAATTGATTGGCTCCAATTCTTGGAGT | |
| | GGTGAATCCGTTAGCGAGGTGCCGCCGGCTTCCATTCAGGTCGAG | |
| | GTGGCCCGGCTCCATGCACCGCGACGCAACGCGGGGAGGCAGACA | |
| | AGGTATAGGGCGGCGCCTACAATCCATGCCAACCCGTTCCATGTGC | |
| | TCGCCGAGGCGGCATAAATCCCCGTGACGATCAGCGGTCCAATGA | |
| | TCGAAGTTAGGCTGGTAAGAGCCGCGAGCGATCCTTGAAGCTGTC | |
| | CCTGATGGTCGTCATCTACCTGCCTGGACAGCATGGCCTGCAACGC | |
| | GGGCATCCCGATGCCGCCGGAAGCGAGAAGAATCATAATGGGGAA | |
| | GGCCATCCAGCCTCGCGTCGGGGAGCTTTTTGCAAAAGCCTAGGCC | |
| | TCCAAAAAAGCCTCCTCACTACTTCTGGAATAGCTCAGAGGCCGA | |
| | GGCGGCCTCGGCCTCTGCATAAATAAAAAAAATTAGTCAGCCATG | |
| 173 | AGCTTGGCCCATTGCATACGTTGTATCCATATCATAATATGTACAT | 20x boxB with |
| | TTATATTGGCTCATGTCCAACATTACCGCCATGTTGACATTGATTAT | Cre |
| | TGACTAGTTATTAATAGTAATCAATTACGGGGTCATTAGTTCATAG | |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGTGCCCAAG | |
| | AAGAAGAGGAAAGTCTCCAACCTGCTGACTGTGCACCAAAACCTG | |
| | CCTGCCCTCCCTGTGGATGCCACCTCTGATGAAGTCAGGAAGAACC | |
| | TGATGGACATGTTCAGGGACAGGCAGGCCTTCTCTGAACACACCT | |
| | GGAAGATGCTCCTGTCTGTGTGCAGATCCTGGGCTGCCTGGTGCAA | |
| | GCTGAACAACAGGAAATGGTTCCCTGCTGAACCTGAGGATGTGAG | |
| | GGACTACCTCCTGTACCTGCAAGCCAGAGGCCTGGCTGTGAAGAC | |
| | CATCCAACAGCACCTGGGCCAGCTCAACATGCTGCACAGGAGATC | |
| | TGGCCTGCCTCGCCCTTCTGACTCCAATGCTGTGTCCCTGGTGATG | |
| | AGGAGAATCAGAAAGGAGAATGTGGATGCTGGGGAGAGAGCCAA | |
| | GCAGGCCCTGGCCTTTGAACGCACTGACTTTGACCAAGTCAGATCC | |
| | CTGATGGAGAACTCTGACAGATGCCAGGACATCAGGAACCTGGCC | |
| | TTCCTGGGCATTGCCTACAACACCCTGCTGCGCATTGCCGAAATTG | |
| | CCAGAATCAGAGTGAAGGACATCTCCCGCACCGATGGTGGGAGAA | |
| | TGCTGATCCACATTGGCAGGACCAAGACCCTGGTGTCCACAGCTG | |
| | GTGTGGAGAAGGCCCTGTCCCTGGGGGTTACCAAGCTGGTGGAGA | |
| | GATGGATCTCTGTGTCTGGTGTGGCTGATGACCCCAACAACTACCT | |
| | GTTCTGCCGGGTCAGAAAGAATGGTGTGGCTGCCCCTTCTGCCACC | |
| | TCCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCAAGTCCAACTACTAAACTGGGGATTCCTGGGCCCTGAA | |
| | GAAGGGCCCCTCGACTAAGTCCAACTACTAAACTGGGCCCTGAAG | |
| | AAGGGCCCATATAGGGCCCTGAAGAAGGGCCCTATCGAGGATATT | |
| | ATCTCGACTAAGTCCAACTACTAAACTGGGCCCTGAAGAAGGGCC | |
| | CATATAGGGCCCTGAAGAAGGGCCCTATCGAGGATATTATCAATC | |
| | TTCCTGCTCAGTAAGCAAGTCCAACTACTAAACTGGGGATTCCTGG | |
| | GCCCTGAAGAAGGGCCCCTCGACTAAGTCCAACTACTAAACTGGG | |
| | CCCTGAAGAAGGGCCCATATAGGGCCCTGAAGAAGGGCCCTATCG | |
| | AGGATATTATCTCGACTAAGTCCAACTACTAAACTGGGCCCTGAAG | |
| | AAGGGCCCATATAGGGCCCTGAAGAAGGGCCCTATCGAGGATATT | |
| | ATCAATCTTCCTGCTCAGTGAAAAAGTCCAACTACTAAACTGGGGA | |
| | TTCCTGGGCCCTGAAGAAGGGCCCCTCGACTAAGTCCAACTACTAA | |
| | ACTGGGCCCTGAAGAAGGGCCCATATAGGGCCCTGAAGAAGGGCC | |
| | CTATCGAGGATATTATCTCGACTAAGTCCAACTACTAAACTGGGCC | |
| | CTGAAGAAGGGCCCATATAGGGCCCTGAAGAAGGGCCCTATCGAG | |
| | GATATTATCAATCTTCCTGCTCAGTTCCCAAGTCCAACTACTAAAC | |
| | TGGGGATTCCTGGGCCCTGAAGAAGGGCCCCTCGACTAAGTCCAA | |
| | CTACTAAACTGGGCCCTGAAGAAGGGCCCATATAGGGCCCTGAAG | |
| | AAGGGCCCTATCGAGGATATTATCTCGACTAAGTCCAACTACTAAA | |
| | CTGGGCCCTGAAGAAGGGCCCATATAGGGCCCTGAAGAAGGGCCC | |
| | TATCGAGGATATTATCAATCTTCCTGCTCAGTGTCGACCTCGAGGG | |
| | CCCAGATCTAATTCACCCCACCAGTGCAGGCTGCCTATCAGAAAGT | |
| | GGTGGCTGGTGTGGCTAATGCCCTGGCCCACAAGTATCACTAAGCT | |
| | CGCTTTCTTGCTGTCCAATTTCTATTAAAGGTTCCTTTGTTCCCTAA | |
| | GTCCAACTACTAAACTGGGGGATATTATGAAGGGCCTTGAGCATCT | |
| | GGATTCTGCCTAATAAAAAACATTTATTTTCATTGCAATGATGTAT | |
| | TTAAATTATTTCTGAATATTTTACTAAAAAGGGAATGTGGGAGGTC | |
| | AGTGCATTTAAAACATAAAGAAATGAAGAGCTAGTTCAAACCTTG | |
| | GGAAAATACACTATATCTTAAACTCCATGAAAGAAGGTGAGGCTG | |
| | CAAACAGCTAATGCACATTGGCAACAGCCCCTGATGCCTATGCCTT | |
| | ATTCATCCCTCAGAAAAGGATTCAAGTAGAGGCTTGATTTGGAGGT | |
| | TAAAGTTTTGCTATGCTGTATTTTACATTACTTATTGTTTTAGCTGT | |
| | CCTCATGAATGTCTTTTCACTACCCATTTGCTTATCCTGCATCTCTC | |
| | AGCCTTGACTCCACTCAGTTCTCTTGCTTAGAGATACCACCTTTCCC | |
| | CTGAAGTGTTCCTTCCATGTTTTACGGCGAGATGGTTTCTCCTCGCC | |
| | TGGCCACTCAGCCTTAGTTGTCTCTGTTGTCTTATAGAGGTCTACTT | |
| | GAAGAAGGAAAAACAGGGGGCATGGTTTGACTGTCCTGTGAGCCC | |
| | TTCTTCCCTGCCTCCCCCACTCACAGTGACCCGGAATCCCTCGACA | |
| | TGGCAGTCTAGATCATTCTTGAAGACGAAAGGGCCTCGTGATACG | |
| | CCTATTTTTATAGGTTAATGTCATGATAATAATGGTTTCTTAGACGT | |
| | CAGGTGGCACTTTTCGGGGAAATGTGCGCGGAACCCCTATTTGTTT | |
| | ATTTTTCTAAATACATTCAAATATGTATCCGCTCATGAGACAATAA | |
| | CCCTGATAAATGCTTCAATAATATTGAAAAAGGAAGAGTATGAGT | |
| | ATTCAACATTTCCGTGTCGCCCTTATTCCCTTTTTTGCGGCATTTTG | |
| | CCTTCCTGTTTTTGCTCACCCAGAAACGCTGGTGAAAGTAAAAGAT | |
| | GCTGAAGATCAGTTGGGTGCACGAGTGGGTTACATCGAACTGGAT | |
| | CTCAACAGCGGTAAGATCCTTGAGAGTTTTCGCCCCGAAGAACGTT | |
| | TTCCAATGATGAGCACTTTTAAAGTTCTGCTATGTGGCGCGGTATT | |
| | ATCCCGTATTGACGCCGGGCAAGAGCAACTCGGTCGCCGCATACA | |
| | CTATTCTCAGAATGACTTGGTTGAGTACTCACCAGTCACAGAAAAG | |
| | CATCTTACGGATGGCATGACAGTAAGAGAATTATGCAGTGCTGCC | |
| | ATAACCATGAGTGATAACACTGCGGCCAACTTACTTCTGACAACG | |
| | ATCGGAGGACCGAAGGAGCTAACCGCTTTTTTGCACAACATGGGG | |
| | GATCATGTAACTCGCCTTGATCGTTGGGAACCGGAGCTGAATGAA | |
| | GCCATACCAAACGACGAGCGTGACACCACGATGCCTGTAGCAATG | |
| | GCAACAACGTTGCGCAAACTATTAACTGGCGAACTACTTACTCTAG | |
| | CTTCCCGGCAACAATTAATAGACTGGATGGAGGCGGATAAAGTTG | |
| | CAGGACCACTTCTGCGCTCGGCCCTTCCGGCTGGCTGGTTTATTGC | |
| | TGATAAATCTGGAGCCGGTGAGCGTGGGTCTCGCGGTATCATTGCA | |
| | GCACTGGGGCCAGATGGTAAGCCCTCCCGTATCGTAGTTATCTACA | |
| | CGACGGGGAGTCAGGCAACTATGGATGAACGAAATAGACAGATCG | |
| | CTGAGATAGGTGCCTCACTGATTAAGCATTGGTAACTGTCAGACCA | |
| | AGTTTACTCATATATACTTTAGATTGATTTAAAACTTCATTTTTAAT | |
| | TTAAAAGGATCTAGGTGAAGATCCTTTTTGATAATCTCATGACCAA | |
| | AATCCCTTAACGTGAGTTTTCGTTCCACTGAGCGTCAGACCCCGTA | |
| | GAAAAGATCAAAGGATCTTCTTGAGATCCTTTTTTTCTGCGCGTAA | |
| | TCTGCTGCTTGCAAACAAAAAAACCACCGCTACCAGCGGTGGTTTG | |
| | TTTGCCGGATCAAGAGCTACCAACTCTTTTTCCGAAGGTAACTGGC | |
| | TTCAGCAGAGCGCAGATACCAAATACTGTTCTTCTAGTGTAGCCGT | |
| | AGTTAGGCCACCACTTCAAGAACTCTGTAGCACCGCCTACATACCT | |
| | CGCTCTGCTAATCCTGTTACCAGTGGCTGCTGCCAGTGGCGATAAG | |
| | TCGTGTCTTACCGGGTTGGACTCAAGACGATAGTTACCGGATAAGG | |
| | CGCAGCGGTCGGGCTGAACGGGGGGTTCGTGCACACAGCCCAGCT | |
| | TGGAGCGAACGACCTACACCGAACTGAGATACCTACAGCGTGAGC | |
| | TATGAGAAAGCGCCACGCTTCCCGAAGGGAGAAAGGCGGACAGGT | |
| | ATCCGGTAAGCGGCAGGGTCGGAACAGGAGAGCGCACGAGGGAG | |
| | CTTCCAGGGGGAAACGCCTGGTATCTTTATAGTCCTGTCGGGTTTC | |
| | GCCACCTCTGACTTGAGCGTCGATTTTTGTGATGCTCGTCAGGGGG | |
| | GCGGAGCCTATGGAAAAACGCCAGCAACGGATGCGCCGCGTGCGG | |
| | CTGCTGGAGATGGCGGACGCGATGGATATGTTCTGCCAAGGGTTG | |
| | GTTTGCGCATTCACAGTTCTCCGCAAGAATTGATTGGCTCCAATTC | |
| | TTGGAGTGGTGAATCCGTTAGCGAGGTGCCGCCGGCTTCCATTCAG | |
| | GTCGAGGTGGCCCGGCTCCATGCACCGCGACGCAACGCGGGGAGG | |
| | CAGACAAGGTATAGGGCGGCGCCTACAATCCATGCCAACCCGTTC | |
| | CATGTGCTCGCCGAGGCGGCATAAATCCCCGTGACGATCAGCGGT | |
| | CCAATGATCGAAGTTAGGCTGGTAAGAGCCGCGAGCGATCCTTGA | |
| | AGCTGTCCCTGATGGTCGTCATCTACCTGCCTGGACAGCATGGCCT | |
| | GCAACGCGGGCATCCCGATGCCGCCGGAAGCGAGAAGAATCATAA | |
| | TGGGGAAGGCCATCCAGCCTCGCGTCGGGGAGCTTTTTGCAAAAG | |
| | CCTAGGCCTCCAAAAAAGCCTCCTCACTACTTCTGGAATAGCTCAG | |
| | AGGCCGAGGCGGCCTCGGCCTCTGCATAAATAAAAAAAATTAGTC | |
| | AGCCATG | |
| 174 | GGATCCTAAGGTACCTAATTGCCTAGAAAACATGAGGATCACCCA | 1x MS2 |
| | TGTCTGCAGGTCGAC | without Cre |
| 175 | GGATCCTAAGGTACCTAATTGCCTAGAAAACATGAGGATCACCCA | 2x MS2 |
| | TGTCTGCAGGTCGACTCTAGAAAACATGAGGATCACCCATGTCTGC | without Cre |
| | AGTATTCCCGGGTTCATTAGATCCGTCGAC | |
| 176 | GGATCCAACCTACAAACGGGTGGAGGATCACCCCACCCGACACTT | 6x MS2 |
| | CACAATCAAGGGGTACAATACACAAGGGTGGAGGAACACCCCACC | without Cre |
| | CTCCAGACACATTACACAGAAATCCAATCAAACAGAAGCACCATC | |
| | AGGGCTTCTGCTACCAAATTTATCTCAAAAAACTACAACAAGGAA | |
| | TCACCATCAGGGATTCCCTGTGCAATATACGTCAAACGAGGGCCA | |
| | CGACGGGAGGACGATCACGCCTCCCGAATATCGGCATGTCTGGCT | |
| | TTCGAATTCAGTGCGTGGAGCATCAGCCCACGCAGCCAATCAGAG | |
| | TCGAATACAAGTCGAC | |
| 177 | GGATCCAACCTACAAACGGGTGGAGGATCACCCCACCCGACACTT | 12x MS2 |
| | CACAATCAAGGGGTACAATACACAAGGGTGGAGGAACACCCCACC | without Cre |
| | CTCCAGACACATTACACAGAAATCCAATCAAACAGAAGCACCATC | |
| | AGGGCTTCTGCTACCAAATTTATCTCAAAAAACTACAACAAGGAA | |
| | TCACCATCAGGGATTCCCTGTGCAATATACGTCAAACGAGGGCCA | |
| | CGACGGGAGGACGATCACGCCTCCCGAATATCGGCATGTCTGGCT | |
| | TTCGAATTCAGTGCGTGGAGCATCAGCCCACGCAGCCAATCAGAG | |
| | TCGAATACAAGTCGACTTTCGCGAAGAGCATCAGCCTTCGCGCCAT | |
| | TCTTACACAAACCACACTCTCCCCTACAGGAACAGCATCAGCGTTC | |
| | CTGCCCAGTACCCAACTCAAGAAAATTTATGTCCCCATGCAGCATC | |
| | AGCGCATGGGCCCCAAGAATACATCCCCAACAAAATCACATCCGA | |
| | GCACCAACAGGGCTCGGAGTGTTGTTTCTTGTCCAACTGGACAAAC | |
| | CCTCCATGGACCATCAGGCCATGGACTCTCACCAACAAGACAAAA | |
| | ACTACTCTTCTCGAAGCAGCATCAGCGCTTCGAAACACTCGACCTC | |
| | GAG | |
| 178 | GGATCCAACCTACAAACGGGTGGAGGATCACCCCACCCGACACTT | 24x MS2 |
| | CACAATCAAGGGGTACAATACACAAGGGTGGAGGAACACCCCACC | without Cre |
| | CTCCAGACACATTACACAGAAATCCAATCAAACAGAAGCACCATC | |
| | AGGGCTTCTGCTACCAAATTTATCTCAAAAAACTACAACAAGGAA | |
| | TCACCATCAGGGATTCCCTGTGCAATATACGTCAAACGAGGGCCA | |
| | CGACGGGAGGACGATCACGCCTCCCGAATATCGGCATGTCTGGCT | |
| | TTCGAATTCAGTGCGTGGAGCATCAGCCCACGCAGCCAATCAGAG | |
| | TCGAATACAAGTCGACTTTCGCGAAGAGCATCAGCCTTCGCGCCAT | |
| | TCTTACACAAACCACACTCTCCCCTACAGGAACAGCATCAGCGTTC | |
| | CTGCCCAGTACCCAACTCAAGAAAATTTATGTCCCCATGCAGCATC | |
| | AGCGCATGGGCCCCAAGAATACATCCCCAACAAAATCACATCCGA | |
| | GCACCAACAGGGCTCGGAGTGTTGTTTCTTGTCCAACTGGACAAAC | |
| | CCTCCATGGACCATCAGGCCATGGACTCTCACCAACAAGACAAAA | |
| | ACTACTCTTCTCGAAGCAGCATCAGCGCTTCGAAACACTCGAGCAT | |
| | ACATTGTGCCTATTTCTTGGGTGGACGATCACGCCACCCATGCTCT | |
| | CACGAATTTCAAAACACGGACAAGGACGAGCACCACCAGGGCTCG | |
| | TCGTTCCACGTCCAATACGATTACTTACCTTTCGGGATCACGATCA | |
| | CGGATCCCGCAGCTACATCACTTCCACTCAGGACATTCAAGCATGC | |
| | ACGATCACGGCATGCTCCACAAGTCTCAACCACAGAAACTACCAA | |
| | ATGGGTTCAGCACCAGCGAACCCACTCCTACCTCAAACCTCTTCCC | |
| | ACAAAACTGGCAAGCAGGATCACCGCTTGCCCATTCCAACATACC | |
| | AAATCAAAAACAATTACTGGTACAGCATCAGCGTACCAGCCCACA | |
| | TCTCTCACTACTATCAAAAACCAAACCGTTCAGCAACAGCGAACG | |
| | GTACACACGGAAAAATCAACTGGTTTACAAATACGAAAGACGATC | |
| | ACGCTTTCGTCCAGCGCAAACTATTACGAAAAACATCCGACGGGA | |
| | AGAGCAACAGCCTTCCCGCGGCGGAAAACCTCACAAAAACACGAC | |
| | AAACGGATGCACGAACACGGCATCCGCCGACAACCCACAAACTTA | |
| | CAACCAGGCAAACGGTGCAGGATCACCGCACCGTACATCAAACAC | |
| | CTCAGATCTCATGTCGA | |
| 179 | AAGTCCAACTACTAAACTGGGGATTCCTGGGCCCTGAAGAAGGGC | 1x boxB |
| | CCCTCGTCGAC | without Cre |
| 180 | AAGTCCAACTACTAAACTGGGGATTCCTGGGCCCTGAAGAAGGGC | 2x boxB |
| | CCCTCGACTAAGTCCAACTACTAAACTGGGCCCTGAAGAAGGGCC | without Cre |
| | CATATAGTCGAC | |
| 181 | AAGTCCAACTACTAAACTGGGGATTCCTGGGCCCTGAAGAAGGGC | 5x boxB |
| | CCCTCGACTAAGTCCAACTACTAAACTGGGCCCTGAAGAAGGGCC | without Cre |
| | CATATAGGGCCCTGAAGAAGGGCCCTATCGAGGATATTATCTCGA | |
| | CTAAGTCCAACTACTAAACTGGGCCCTGAAGAAGGGCCCATATAG | |
| | GGCCCTGAAGAAGGGCCCTATCGAGGATATTATCAATCTTCCTGCT | |
| | CAGTGTCGAC | |
| 182 | AAGTCCAACTACTAAACTGGGGATTCCTGGGCCCTGAAGAAGGGC | 10x boxB |
| | CCCTCGACTAAGTCCAACTACTAAACTGGGCCCTGAAGAAGGGCC | without Cre |
| | CATATAGGGCCCTGAAGAAGGGCCCTATCGAGGATATTATCTCGA | |
| | CTAAGTCCAACTACTAAACTGGGCCCTGAAGAAGGGCCCATATAG | |
| | GGCCCTGAAGAAGGGCCCTATCGAGGATATTATCAATCTTCCTGCT | |
| | CAGTAAGCAAGTCCAACTACTAAACTGGGGATTCCTGGGCCCTGA | |
| | AGAAGGGCCCCTCGACTAAGTCCAACTACTAAACTGGGCCCTGAA | |
| | GAAGGGCCCATATAGGGCCCTGAAGAAGGGCCCTATCGAGGATAT | |
| | TATCTCGACTAAGTCCAACTACTAAACTGGGCCCTGAAGAAGGGC | |
| | CCATATAGGGCCCTGAAGAAGGGCCCTATCGAGGATATTATCAAT | |
| | CTTCCTGCTCAGTGTCGAC | |
| 183 | AAGTCCAACTACTAAACTGGGGATTCCTGGGCCCTGAAGAAGGGC | 15x boxB |
| | CCCTCGACTAAGTCCAACTACTAAACTGGGCCCTGAAGAAGGGCC | without Cre |
| | CATATAGGGCCCTGAAGAAGGGCCCTATCGAGGATATTATCTCGA | |
| | CTAAGTCCAACTACTAAACTGGGCCCTGAAGAAGGGCCCATATAG | |
| | GGCCCTGAAGAAGGGCCCTATCGAGGATATTATCAATCTTCCTGCT | |
| | CAGTAAGCAAGTCCAACTACTAAACTGGGGATTCCTGGGCCCTGA | |
| | AGAAGGGCCCCTCGACTAAGTCCAACTACTAAACTGGGCCCTGAA | |
| | GAAGGGCCCATATAGGGCCCTGAAGAAGGGCCCTATCGAGGATAT | |
| | TATCTCGACTAAGTCCAACTACTAAACTGGGCCCTGAAGAAGGGC | |
| | CCATATAGGGCCCTGAAGAAGGGCCCTATCGAGGATATTATCAAT | |
| | CTTCCTGCTCAGTGAAAAAGTCCAACTACTAAACTGGGGATTCCTG | |
| | GGCCCTGAAGAAGGGCCCCTCGACTAAGTCCAACTACTAAACTGG | |
| | GCCCTGAAGAAGGGCCCATATAGGGCCCTGAAGAAGGGCCCTATC | |
| | GAGGATATTATCTCGACTAAGTCCAACTACTAAACTGGGCCCTGAA | |
| | GAAGGGCCCATATAGGGCCCTGAAGAAGGGCCCTATCGAGGATAT | |
| | TATCAATCTTCCTGCTCAGTGTCGAC | |
| 184 | AAGTCCAACTACTAAACTGGGGATTCCTGGGCCCTGAAGAAGGGC | 20x boxB |
| | CCCTCGACTAAGTCCAACTACTAAACTGGGCCCTGAAGAAGGGCC | without Cre |
| | CATATAGGGCCCTGAAGAAGGGCCCTATCGAGGATATTATCTCGA | |
| | CTAAGTCCAACTACTAAACTGGGCCCTGAAGAAGGGCCCATATAG | |
| | GGCCCTGAAGAAGGGCCCTATCGAGGATATTATCAATCTTCCTGCT | |
| | CAGTAAGCAAGTCCAACTACTAAACTGGGGATTCCTGGGCCCTGA | |
| | AGAAGGGCCCCTCGACTAAGTCCAACTACTAAACTGGGCCCTGAA | |
| | GAAGGGCCCATATAGGGCCCTGAAGAAGGGCCCTATCGAGGATAT | |
| | TATCTCGACTAAGTCCAACTACTAAACTGGGCCCTGAAGAAGGGC | |
| | CCATATAGGGCCCTGAAGAAGGGCCCTATCGAGGATATTATCAAT | |
| | CTTCCTGCTCAGTGAAAAAGTCCAACTACTAAACTGGGGATTCCTG | |
| | GGCCCTGAAGAAGGGCCCCTCGACTAAGTCCAACTACTAAACTGG | |
| | GCCCTGAAGAAGGGCCCATATAGGGCCCTGAAGAAGGGCCCTATC | |
| | GAGGATATTATCTCGACTAAGTCCAACTACTAAACTGGGCCCTGAA | |
| | GAAGGGCCCATATAGGGCCCTGAAGAAGGGCCCTATCGAGGATAT | |
| | TATCAATCTTCCTGCTCAGTTCCCAAGTCCAACTACTAAACTGGGG | |
| | ATTCCTGGGCCCTGAAGAAGGGCCCCTCGACTAAGTCCAACTACTA | |
| | AACTGGGCCCTGAAGAAGGGCCCATATAGGGCCCTGAAGAAGGGC | |
| | CCTATCGAGGATATTATCTCGACTAAGTCCAACTACTAAACTGGGC | |
| | CCTGAAGAAGGGCCCATATAGGGCCCTGAAGAAGGGCCCTATCGA | |
| | GGATATTATCAATCTTCCTGCTCAGTGTCGAC | |
| 185 | X₁X₂X₃X₄AX₅X₆AX₇PAX₈X₉X₁₀X₁₁X₁₂X₁₃ | MS2 stem loop |
| | | (the following |
| | | pairs are |
| | | complementary |
| | | X₁ and X₁₃, |
| | | X₂ and X₁₂, X₃ |
| | | and X₁₁, X₄ |
| | | and X₁₀, X₅ |
| | | and X₉, X₆ and |
| | | X₈ and P is a |
| | | pyrimidine) |
| 186 | gggccctgaagaagggcc | BoxB stem |
| | | loop |
| 187 | MDAQTRRRERRAEKQAQWKAAN | Phage lambda |
| | | N protein |
| 188 | MDAQTRRRERRAEKQAQWKAAN | Mutated phage |
| | | lambda N |
| | | protein |
| 189 | MKCLLYLAFLFIGVNCKFTIVFPHNQKGNWKNVPSNYHYCPSSSDLN | VSV-G |
| | WHNDLIGTALQVKMPKSHKAIQADGWMCHASKWVTTCDFRWYGPK | |
| | YITHSIRSFTPSVEQCKESIEQTKQGTWLNPGFPPQSCGYATVTDAEAV | |
| | IVQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKM | |
| 190 | MGARASVLSGGELDKWEKIRLRPGGKKQYKLKHIVWASRELERFAV | MA-td-MS2_{cp} |
| | NPGLLETSEGCRQILGQLQPSLQTGSEELRSLYNTIAVLYCVHQRIDVK | (aa) |
| | DTKEALDKIEEEQNKSKKKAQQAAADTGNNSQVSQNYPIVQMASNF | |
| | TQFVLVDNGGTGDVTVAPSNFANGIAEWISSNSRSQAYKVTCSVRQS | |
| | SAQNRKYTIKVEVPKGAWRSYLNMELTIPIFATNSDCELIVKAMQGLL | |
| | KDGNPIPSAIAANSGIYAMASNFTQFVLVDNGGTGDVTVAPSNFANGI | |
| | AEWISSNSRSQAYKVTCSVRQSSAQNRKYTIKVEVPKGAWRSYLNME | |
| | LTIPIFATNSDCELIVKAMQGLLKDGNPIPSAIAANSGIY | |
| 191 | GARASVLSGGELDKWEKIRLRPGGKKQYKLKHIVWASRELERFAVNP | MA-td-MS2_{cp} |
| | GLLETSEGCRQILGQLQPSLQTGSEELRSLYNTIAVLYCVHQRIDVKDT | no methionine |
| | KEALDKIEEEQNKSKKKAQQAAADTGNNSQVSQNYPIVQMASNFTQF | (aa) |
| | VLVDNGGTGDVTVAPSNFANGIAEWISSNSRSQAYKVTCSVRQSSAQ | |
| | NRKYTIKVEVPKGAWRSYLNMELTIPIFATNSDCELIVKAMQGLLKD | |
| | GNPIPSAIAANSGIYAMASNFTQFVLVDNGGTGDVTVAPSNFANGIAE | |
| | WISSNSRSQAYKVTCSVRQSSAQNRKYTIKVEVPKGAWRSYLNMELT | |
| | IPIFATNSDCELIVKAMQGLLKDGNPIPSAIAANSGIY | |
| 192 | KCLLYLAFLFIGVNCKFTIVFPHNQKGNWKNVPSNYHYCPSSSDLNW | VSVG-MS2_{cp} |
| | HNDLIGTALQVKMPKSHKAIQADGWMCHASKWVTTCDFRWYGPKY | no methionine |
| | ITHSIRSFTPSVEQCKESIEQTKQGTWLNPGFPPQSCGYATVTDAEAVI | aa |
| | VQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKMASNFTQFVLVDNGGT | |
| | GDVTVAPSNFANGIAEWISSNSRSQAYKVTCSVRQSSAQNRKYTIKVE | |
| | VPKGAWRSYLNMELTIPIFATNSDCELIVKAMQGLLKDGNPIPSAIAA | |
| | NSGIY | |
| 193 | KCLLYLAFLFIGVNCKFTIVFPHNQKGNWKNVPSNYHYCPSSSDLNW | VSVG-td- |
| | HNDLIGTALQVKMPKSHKAIQADGWMCHASKWVTTCDFRWYGPKY | MS2_{cp} no |
| | ITHSIRSFTPSVEQCKESIEQTKQGTWLNPGFPPQSCGYATVTDAEAVI | methionine |
| | VQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKMASNFTQFVLVDNGGT | |
| | GDVTVAPSNFANGIAEWISSNSRSQAYKVTCSVRQSSAQNRKYTIKVE | |
| | VPKGAWRSYLNMELTIPIFATNSDCELIVKAMQGLLKDGNPIPSAIAA | |
| | NSGIYAMASNFTQFVLVDNGGTGDVTVAPSNFANGIAEWISSNSRSQ | |
| | AYKVTCSVRQSSAQNRKYTIKVEVPKGAWRSYLNMELTIPIFATNSDC | |
| | ELIVKAMQGLLKDGNPIPSAIAANSGIY | |
| 194 | GARASVLSGGELDKWEKIRLRPGGKKQYKLKHIVWASRELERFAVNP | MA-λN no |
| | GLLETSEGCRQILGQLQPSLQTGSEELRSLYNTIAVLYCVHQRIDVKDT | methionine |
| | KEALDKIEEEQNKSKKKAQQAAADTGNNSQVSQNYPIVQMDAQTRR | |
| | RERRAEKQAQWKAAN | |
| 195 | GARASVLSGGELDKWEKIRLRPGGKKQYKLKHIVWASRELERFAVNP | MA-mutAN no |
| | GLLETSEGCRQILGQLQPSLQTGSEELRSLYNTIAVLYCVHQRIDVKDT | methionine |
| | KEALDKIEEEQNKSKKKAQQAAADTGNNSQVSQNYPIVQGNARTRR | |
| | RERRAEKQAQWKAAN | |
| 196 | KCLLYLAFLFIGVNCKFTIVFPHNQKGNWKNVPSNYHYCPSSSDLNW | VSVG-λN no |
| | HNDLIGTALQVKMPKSHKAIQADGWMCHASKWVTTCDFRWYGPKY | methionine |
| | ITHSIRSFTPSVEQCKESIEQTKQGTWLNPGFPPQSCGYATVTDAEAVI | |
| | VQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKMDAQTRRRERRAEKQA | |
| | QWKAAN | |
| 197 | KCLLYLAFLFIGVNCKFTIVFPHNQKGNWKNVPSNYHYCPSSSDLNW | VSVG-mutλN |
| | HNDLIGTALQVKMPKSHKAIQADGWMCHASKWVTTCDFRWYGPKY | no methionine |
| | ITHSIRSFTPSVEQCKESIEQTKQGTWLNPGFPPQSCGYATVTDAEAVI | |
| | VQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKGNARTRRRERRAEKQA | |
| | QWKAAN | |
| 198 | MASNFTQFVLVDNGGTGDVTVAPSNFANGIAEWISSNSRSQAYKVTC | tdMS2 |
| | SVRQSSAQNRKYTIKVEVPKGAWRSYLNMELTIPIFATNSDCELIVKA | |
| | MQGLLKDGNPIPSAIAANSGIYAMASNFTQFVLVDNGGTGDVTVAPS | |
| | NFANGIAEWISSNSRSQAYKVTCSVRQSSAQNRKYTIKVEVPKGAWR | |
| | SYLNMELTIPIFATNSDCELIVKAMQGLLKDGNPIPSAIAANSGIY | |
| 199 | KCLLYLAFLFIGVNCKFTIVFPHNQKGNWKNVPSNYHYCPSSSDLNW | VSV-G |
| | HNDLIGTALQVKMPKSHKAIQADGWMCHASKWVTTCDFRWYGPKY | |
| | ITHSIRSFTPSVEQCKESIEQTKQGTWLNPGFPPQSCGYATVTDAEAVI | |
| | VQVTPHHVLVDEYTGEWVDSQFINGKCSNYICPTVHNSTTWHSDYK | |
| | VKGLCDSNLISMDITFFSEDGELSSLGKEGTGFRSNYFAYETGGKACK | |
| | MQYCKHWGVRLPSGVWFEMADKDLFAAARFPECPEGSSISAPSQTSV | |
| | DVSLIQDVERILDYSLCQETWSKIRAGLPISPVDLSYLAPKNPGTGPAF | |
| | TIINGTLKYFETRYIRVDIAAPILSRMVGMISGTTTERELWDDWAPYED | |
| | VEIGPNGVLRTSSGYKFPLYMIGHGMLDSDLHLSSKAQVFEHPHIQDA | |
| | ASQLPDDESLFFGDTGLSKNPIELVEGWFSSWKSSIASFFFIIGLIIGLFL | |
| | VLRVGIHLCIKLKHTKKRQIYTDIEMNRLGKM | |
| 200 | TGGAAGGGCTAATTCACTCCCAAAGAAGACAAGATATCCTTGATC | GAG-T2A- |
| | TGTGGATCTACCACACACAAGGCTACTTCCCTGATTAGCAGAACTA | EGFP |
| | CACACCAGGGCCAGGGGTCAGATATCCACTGACCTTTGGATGGTG | |
| | CTACAAGCTAGTACCAGTTGAGCCAGATAAGGTAGAAGAGGCCAA | |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGGTGAGTACGCCAAAAATTTTGACTAGCGG | |
| | AGGCTAGAAGGAGAGAGATGGGTGCGAGAGCGTCGGTATTAAGC | |
| | GGGGGAGAATTAGATAAATGGGAAAAAATTCGGTTAAGGCCAGG | |
| | GGGAAAGAAACAATATAAACTAAAACATATAGTATGGGCAAGCA | |
| | GGGAGCTAGAACGATTCGCAGTTAATCCTGGCCTTTTAGAGACATC | |
| | AGAAGGCTGTAGACAAATACTGGGACAGCTACAACCATCCCTTCA | |
| | GACAGGATCAGAAGAACTTAGATCATTATATAATACAATAGCAGT | |
| | CCTCTATTGTGTGCATCAAAGGATAGATGTAAAAGACACCAAGGA | |
| | AGCCTTAGATAAGATAGAGGAAGAGCAAAACAAAAGTAAGAAAA | |
| | AGGCACAGCAAGCAGGTAGTGGCGAGGGCAGAGGAAGTCTTCTAA | |
| | CATGCGGTGACGTGGAGGAGAATCCCGGCCCTATGGTGCCCAAGA | |
| | AGAAGAGGAAAGTCTCCAACCTGCTGACTGTGCACCAAAACCTGC | |
| | CTGCCCTCCCTGTGGATGCCACCTCTGATGAAGTCAGGAAGAACCT | |
| | GATGGACATGTTCAGGGACAGGCAGGCCTTCTCTGAACACACCTG | |
| | GAAGATGCTCCTGTCTGTGTGCAGATCCTGGGCTGCCTGGTGCAAG | |
| | CTGAACAACAGGAAATGGTTCCCTGCTGAACCTGAGGATGTGAGG | |
| | GACTACCTCCTGTACCTGCAAGCCAGAGGCCTGGCTGTGAAGACC | |
| | ATCCAACAGCACCTGGGCCAGCTCAACATGCTGCACAGGAGATCT | |
| | GGCCTGCCTCGCCCTTCTGACTCCAATGCTGTGTCCCTGGTGATGA | |
| | GGAGAATCAGAAAGGAGAATGTGGATGCTGGGGAGAGAGCCAAG | |
| | CAGGCCCTGGCCTTTGAACGCACTGACTTTGACCAAGTCAGATCCC | |
| | TGATGGAGAACTCTGACAGATGCCAGGACATCAGGAACCTGGCCT | |
| | TCCTGGGCATTGCCTACAACACCCTGCTGCGCATTGCCGAAATTGC | |
| | CAGAATCAGAGTGAAGGACATCTCCCGCACCGATGGTGGGAGAAT | |
| | GCTGATCCACATTGGCAGGACCAAGACCCTGGTGTCCACAGCTGG | |
| | TGTGGAGAAGGCCCTGTCCCTGGGGGTTACCAAGCTGGTGGAGAG | |
| | ATGGATCTCTGTGTCTGGTGTGGCTGATGACCCCAACAACTACCTG | |
| | TTCTGCCGGGTCAGAAAGAATGGTGTGGCTGCCCCTTCTGCCACCT | |
| | CCCAACTGTCCACCCGGGCCCTGGAAGGGATCTTTGAGGCCACCC | |
| | ACCGCCTGATCTATGGTGCCAAGGATGACTCTGGGCAGAGATACC | |
| | TGGCCTGGTCTGGCCACTCTGCCAGAGTGGGTGCTGCCAGGGACAT | |
| | GGCCAGGGCTGGTGTGTCCATCCCTGAAATCATGCAGGCTGGTGG | |
| | CTGGACCAATGTGAACATTGTGATGAACTACATCAGAAACCTGGA | |
| | CTCTGAGACTGGGGCCATGGTGAGGCTGCTCGAGGATGGGGACTG | |
| | AGGATCCTAATCAACCTCTGGATTACAAAATTTGTGAAAGATTGAC | |
| | TGGTATTCTTAACTATGTTGCTCCTTTTACGCTATGTGGATACGCTG | |
| | CTTTAATGCCTTTGTATCATGCTATTGCTTCCCGTATGGCTTTCATT | |
| | TTCTCCTCCTTGTATAAATCCTGGTTGCTGTCTCTTTATGAGGAGTT | |
| | GTGGCCCGTTGTCAGGCAACGTGGCGTGGTGTGCACTGTGTTTGCT | |
| | GACGCAACCCCCACTGGTTGGGGCATTGCCACCACCTGTCAGCTCC | |
| | TTTCCGGGACTTTCGCTTTCCCCCTCCCTATTGCCACGGCGGAACTC | |
| | ATCGCCGCCTGCCTTGCCCGCTGCTGGACAGGGGCTCGGCTGTTGG | |
| | GCACTGACAATTCCGTGGTGTTGTCGGGGAAATCATCGTCCTTTCC | |
| | TTGGCTGCTCGCCTGTGTTGCCACCTGGATTCTGCGCGGGACGTCC | |
| | TTCTGCTACGTCCCTTCGGCCCTCAATCCAGCGGACCTTCCTTCCCG | |
| | CGGCCTGCTGCCGGCTCTGCGGCCTCTTCCGCGTCTTCGCCTTCGCC | |
| | CTCAGACGAGTCGGATCTCCCTTTGGGCCGCCTCCCCGCCTGAGAT | |
| | CCTTTAAGACCAATGACTTACAAGGCAGCTGTAGATCTTAGCCACT | |
| | TTTTAAAAGAAAAGGGGGGACTGGAAGGGCTAATTCACTCCCAAC | |
| | GAAGACAAGATCTGCTTTTTGCTTGTACTGGGTCTCTCTGGTTAGA | |
| | CCAGATCTGAGCCTGGGAGCTCTCTGGCTAACTAGGGAACCCACT | |
| | GCTTAAGCCTCAATAAAGCTTGCCTTGAGTGCTTCAAGTAGTGTGT | |
| | GCCCGTCTGTTGTGTGACTCTGGTAACTAGAGATCCCTCAGACCCT | |
| | TTTAGTCAGTGTGGAAAATCTCTAGCAGTAGTAGTTCATGTCATCT | |
| | TATTATTCAGTATTTATAACTTGCAAAGAAATGAATATCAGAGAGT | |
| | GAGAGGCCCGGGTTAATTAAGGAAAGGGCTAGATCATTCTTGAAG | |
| | ACGAAAGGGCCTCGTGATACGCCTATTTTTATAGGTTAATGTCATG | |
| | ATAATAATGGTTTCTTAGACGTCAGGTGGCACTTTTCGGGGAAATG | |
| | TGCGCGGAACCCCTATTTGTTTATTTTTCTAAATACATTCAAATATG | |
| | TATCCGCTCATGAGACAATAACCCTGATAAATGCTTCAATAATATT | |
| | GAAAAAGGAAGAGTATGAGTATTCAACATTTCCGTGTCGCCCTTAT | |
| | TCCCTTTTTTGCGGCATTTTGCCTTCCTGTTTTTGCTCACCCAGAAA | |
| | CGCTGGTGAAAGTAAAAGATGCTGAAGATCAGTTGGGTGCACGAG | |
| | TGGGTTACATCGAACTGGATCTCAACAGCGGTAAGATCCTTGAGA | |
| | GTTTTCGCCCCGAAGAACGTTTTCCAATGATGAGCACTTTTAAAGT | |
| | TCTGCTATGTGGCGCGGTATTATCCCGTGTTGACGCCGGGCAAGAG | |
| | CAACTCGGTCGCCGCATACACTATTCTCAGAATGACTTGGTTGAGT | |
| | ACTCACCAGTCACAGAAAAGCATCTTACGGATGGCATGACAGTAA | |
| | GAGAATTATGCAGTGCTGCCATAACCATGAGTGATAACACTGCGG | |
| | CCAACTTACTTCTGACAACGATCGGAGGACCGAAGGAGCTAACCG | |
| | CTTTTTTGCACAACATGGGGGATCATGTAACTCGCCTTGATCGTTG | |
| | GGAACCGGAGCTGAATGAAGCCATACCAAACGACGAGCGTGACAC | |
| | CACGATGCCTGTAGCAATGGCAACAACGTTGCGCAAACTATTAAC | |
| | TGGCGAACTACTTACTCTAGCTTCCCGGCAACAATTAATAGACTGG | |
| | ATGGAGGCGGATAAAGTTGCAGGACCACTTCTGCGCTCGGCCCTTC | |
| | CGGCTGGCTGGTTTATTGCTGATAAATCTGGAGCCGGTGAGCGTGG | |
| | GTCTCGCGGTATCATTGCAGCACTGGGGCCAGATGGTAAGCCCTCC | |
| | CGTATCGTAGTTATCTACACGACGGGGAGTCAGGCAACTATGGAT | |
| | GAACGAAATAGACAGATCGCTGAGATAGGTGCCTCACTGATTAAG | |
| | CATTGGTAACTGTCAGACCAAGTTTACTCATATATACTTTAGATTG | |
| | ATTTAAAACTTCATTTTTAATTTAAAAGGATCTAGGTGAAGATCCT | |
| | TTTTGATAATCTCATGACCAAAATCCCTTAACGTGAGTTTTCGTTCC | |
| | ACTGAGCGTCAGACCCCGTAGAAAAGATCAAAGGATCTTCTTGAG | |
| | ATCCTTTTTTTCTGCGCGTAATCTGCTGCTTGCAAACAAAAAAACC | |
| | ACCGCTACCAGCGGTGGTTTGTTTGCCGGATCAAGAGCTACCAACT | |
| | CTTTTTCCGAAGGTAACTGGCTTCAGCAGAGCGCAGATACCAAATA | |
| | CTGTTCTTCTAGTGTAGCCGTAGTTAGGCCACCACTTCAAGAACTC | |
| | TGTAGCACCGCCTACATACCTCGCTCTGCTAATCCTGTTACCAGTG | |
| | GCTGCTGCCAGTGGCGATAAGTCGTGTCTTACCGGGTTGGACTCAA | |
| | GACGATAGTTACCGGATAAGGCGCAGCGGTCGGGCTGAACGGGGG | |
| | GTTCGTGCACACAGCCCAGCTTGGAGCGAACGACCTACACCGAAC | |
| | TGAGATACCTACAGCGTGAGCTATGAGAAAGCGCCACGCTTCCCG | |
| | AAGGGAGAAAGGCGGACAGGTATCCGGTAAGCGGCAGGGTCGGA | |
| | ACAGGAGAGCGCACGAGGGAGCTTCCAGGGGGAAACGCCTGGTAT | |
| | CTTTATAGTCCTGTCGGGTTTCGCCACCTCTGACTTGAGCGTCGATT | |
| | TTTGTGATGCTCGTCAGGGGGGCGGAGCCTATGGAAAAACGCCAG | |
| | CAACGCGGCCTTTTTACGGTTCCTGGCCTTTTGCTGGCCTTTTGCTC | |
| | ACATGTTCTTTCCTGCGTTATCCCCTGATTCTGTGGATAACCGTATT | |
| | ACCGCCTTTGAGTGAGCTGATACCGCTCGCCGCAGCCGAACGACC | |
| | GAGCGCAGCGAGTCAGTGAGCGAGGAAGCGGAAGAGCGCCCAAT | |
| | ACGCAAACCGCCTCTCCCCGCGCGTTGGCCGATTCATTAATGCAGC | |
| | AAGCTCATGGCTGACTAATTTTTTTTATTTATGCAGAGGCCGAGGC | |
| | CGCCTCGGCCTCTGAGCTATTCCAGAAGTAGTGAGGAGGCTTTTTT | |
| | GGAGGCCTAGGCTTTTGCAAAAAGCTCCCCGTGGCACGACAGGTT | |
| | TCCCGACTGGAAAGCGGGCAGTGAGCGCAACGCAATTAATGTGAG | |
| | TTAGCTCACTCATTAGGCACCCCAGGCTTTACACTTTATGCTTCCG | |
| | GCTCGTATGTTGTGTGGAATTGTGAGCGGATAACAATTTCACACAG | |
| | GAAACAGCTATGACATGATTACGAATTTCACAAATAAAGCATTTTT | |
| | TTCACTGCATTCTAGTTGTGGTTTGTCCAAACTCATCAATGTATCTT | |
| | ATCATGTCTGGATCAACTGGATAACTCAAGCTAACCAAAATCATCC | |
| | CAAACTTCCCACCCCATACCCTATTACCACTGCCAATTACCTGTGG | |
| | TTTCATTTACTCTAAACCTGTGATTCCTCTGAATTATTTTCATTTTA | |
| | AAGAAATTGTATTTGTTAAATATGTACTACAAACTTAGTAGT | |
| 201 | MVPKKKRKVSNLLTVHQNLPALPVDATSDEVRKNLMDMFRDRQAFS | Nuclear |
| | EHTWKMLLSVCRSWAAWCKLNNRKWFPAEPEDVRDYLLYLQARGL | localization |
| | AVKTIQQHLGQLNMLHRRSGLPRPSDSNAVSLVMRRIRKENVDAGER | signal Cre |
| | AKQALAFERTDFDQVRSLMENSDRCQDIRNLAFLGIAYNTLLRIAEIA | |
| | RIRVKDISRTDGGRMLIHIGRTKTLVSTAGVEKALSLGVTKLVERWIS | |
| | VSGVADDPNNYLFCRVRKNGVAAPSATSQLSTRALEGIFEATHRLIYG | |
| | AKDDSGQRYLAWSGHSARVGAARDMARAGVSIPEIMQAGGWTNVN | |
| | IVMNYIRNLDSETGAMVRLLEDGD | |
| 202 | MSNLLTVHQNLPALPVDATSDEVRKNLMDMFRDRQAFSEHTWKML | Cre |
| | LSVCRSWAAWCKLNNRKWFPAEPEDVRDYLLYLQARGLAVKTIQQH | |
| | LGQLNMLHRRSGLPRPSDSNAVSLVMRRIRKENVDAGERAKQALAFE | |
| | RTDFDQVRSLMENSDRCQDIRNLAFLGIAYNTLLRIAEIARIRVKDISR | |
| | TDGGRMLIHIGRTKTLVSTAGVEKALSLGVTKLVERWISVSGVADDP | |
| | NNYLFCRVRKNGVAAPSATSQLSTRALEGIFEATHRLIYGAKDDSGQR | |
| | YLAWSGHSARVGAARDMARAGVSIPEIMQAGGWTNVNIVMNYIRNL | |
| | DSETGAMVRLLEDGD | |
| 203 | MVSKGEELFTGVVPILVELDGDVNGHKFSVSGEGEGDATYGKLTLKF | EGFP |
| | ICTTGKLPVPWPTLVTTLTYGVQCFSRYPDHMKQHDFFKSAMPEGYV | |
| | QERTIFFKDDGNYKTRAEVKFEGDTLVNRIELKGIDFKEDGNILGHKL | |
| | EYNYNSHNVYIMADKQKNGIKVNFKIRHNIEDGSVQLADHYQQNTPI | |
| | GDGPVLLPDNHYLSTQSALSKDPNEKRDHMVLLEFVTAAGITLGMDE | |
| | LYK | |
| 204 | TGGAAGGGCTAATTCACTCCCAAAGAAGACAAGATATCCTTGATC | pMA2 N-term |
| | TGTGGATCTACCACACACAAGGCTACTTCCCTGATTAGCAGAACTA | mutated MSD |
| | CACACCAGGGCCAGGGGTCAGATATCCACTGACCTTTGGATGGTG | GAG-T2A |
| | CTACAAGCTAGTACCAGTTGAGCCAGATAAGGTAGAAGAGGCCAA | |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGCAGAGTACGCCAAAAATTTTGACTAGCGG | |
| | AGGCTAGAAGGAGAGAGATGGGTGCGAGAGCGTCGGTATTAAGC | |
| | GGGGGAGAATTAGATAAATGGGAAAAAATTCGGTTAAGGCCAGG | |
| | GGGAAAGAAACAATATAAACTAAAACATATAGTATGGGCAAGCA | |
| | GGGAGCTAGAACGATTCGCAGTTAATCCTGGCCTTTTAGAGACATC | |
| | AGAAGGCTGTAGACAAATACTGGGACAGCTACAACCATCCCTTCA | |
| | GACAGGATCAGAAGAACTTAGATCATTATATAATACAATAGCAGT | |
| | CCTCTATTGTGTGCATCAAAGGATAGATGTAAAAGACACCAAGGA | |
| | AGCCTTAGATAAGATAGAGGAAGAGCAAAACAAAAGTAAGAAAA | |
| | AGGCACAGCAAGCAGGTAGTGGCGAGGGCAGAGGAAGTCTTCTAA | |
| | CATGCGGTGACGTGGAGGAGAATCCCGGCCCTTGAGGATCCTAAT | |
| | CAACCTCTGGATTACAAAATTTGTGAAAGATTGACTGGTATTCTTA | |
| | ACTATGTTGCTCCTTTTACGCTATGTGGATACGCTGCTTTAATGCCT | |
| | TTGTATCATGCTATTGCTTCCCGTATGGCTTTCATTTTCTCCTCCTTG | |
| | TATAAATCCTGGTTGCTGTCTCTTTATGAGGAGTTGTGGCCCGTTGT | |
| | CAGGCAACGTGGCGTGGTGTGCACTGTGTTTGCTGACGCAACCCCC | |
| | ACTGGTTGGGGCATTGCCACCACCTGTCAGCTCCTTTCCGGGACTT | |
| | TCGCTTTCCCCCTCCCTATTGCCACGGCGGAACTCATCGCCGCCTG | |
| | CCTTGCCCGCTGCTGGACAGGGGCTCGGCTGTTGGGCACTGACAAT | |
| | TCCGTGGTGTTGTCGGGGAAATCATCGTCCTTTCCTTGGCTGCTCG | |
| | CCTGTGTTGCCACCTGGATTCTGCGCGGGACGTCCTTCTGCTACGT | |
| | CCCTTCGGCCCTCAATCCAGCGGACCTTCCTTCCCGCGGCCTGCTG | |
| | CCGGCTCTGCGGCCTCTTCCGCGTCTTCGCCTTCGCCCTCAGACGA | |
| | GTCGGATCTCCCTTTGGGCCGCCTCCCCGCCTGAGATCCTTTAAGA | |
| | CCAATGACTTACAAGGCAGCTGTAGATCTTAGCCACTTTTTAAAAG | |
| | AAAAGGGGGGACTGGAAGGGCTAATTCACTCCCAACGAAGACAA | |
| | GATCTGCTTTTTGCTTGTACTGGGTCTCTCTGGTTAGACCAGATCTG | |
| | AGCCTGGGAGCTCTCTGGCTAACTAGGGAACCCACTGCTTAAGCCT | |
| | CAATAAAGCTTGCCTTGAGTGCTTCAAGTAGTGTGTGCCCGTCTGT | |
| | TGTGTGACTCTGGTAACTAGAGATCCCTCAGACCCTTTTAGTCAGT | |
| | GTGGAAAATCTCTAGCAGTAGTAGTTCATGTCATCTTATTATTCAG | |
| | TATTTATAACTTGCAAAGAAATGAATATCAGAGAGTGAGAGGCCC | |
| | GGGTTAATTAAGGAAAGGGCTAGATCATTCTTGAAGACGAAAGGG | |
| | CCTCGTGATACGCCTATTTTTATAGGTTAATGTCATGATAATAATG | |
| | GTTTCTTAGACGTCAGGTGGCACTTTTCGGGGAAATGTGCGCGGAA | |
| | CCCCTATTTGTTTATTTTTCTAAATACATTCAAATATGTATCCGCTC | |
| | ATGAGACAATAACCCTGATAAATGCTTCAATAATATTGAAAAAGG | |
| | AAGAGTATGAGTATTCAACATTTCCGTGTCGCCCTTATTCCCTTTTT | |
| | TGCGGCATTTTGCCTTCCTGTTTTTGCTCACCCAGAAACGCTGGTG | |
| | AAAGTAAAAGATGCTGAAGATCAGTTGGGTGCACGAGTGGGTTAC | |
| | ATCGAACTGGATCTCAACAGCGGTAAGATCCTTGAGAGTTTTCGCC | |
| | CCGAAGAACGTTTTCCAATGATGAGCACTTTTAAAGTTCTGCTATG | |
| | TGGCGCGGTATTATCCCGTGTTGACGCCGGGCAAGAGCAACTCGG | |
| | TCGCCGCATACACTATTCTCAGAATGACTTGGTTGAGTACTCACCA | |
| | GTCACAGAAAAGCATCTTACGGATGGCATGACAGTAAGAGAATTA | |
| | TGCAGTGCTGCCATAACCATGAGTGATAACACTGCGGCCAACTTAC | |
| | TTCTGACAACGATCGGAGGACCGAAGGAGCTAACCGCTTTTTTGCA | |
| | CAACATGGGGGATCATGTAACTCGCCTTGATCGTTGGGAACCGGA | |
| | GCTGAATGAAGCCATACCAAACGACGAGCGTGACACCACGATGCC | |
| | TGTAGCAATGGCAACAACGTTGCGCAAACTATTAACTGGCGAACT | |
| | ACTTACTCTAGCTTCCCGGCAACAATTAATAGACTGGATGGAGGCG | |
| | GATAAAGTTGCAGGACCACTTCTGCGCTCGGCCCTTCCGGCTGGCT | |
| | GGTTTATTGCTGATAAATCTGGAGCCGGTGAGCGTGGGTCTCGCGG | |
| | TATCATTGCAGCACTGGGGCCAGATGGTAAGCCCTCCCGTATCGTA | |
| | GTTATCTACACGACGGGGAGTCAGGCAACTATGGATGAACGAAAT | |
| | AGACAGATCGCTGAGATAGGTGCCTCACTGATTAAGCATTGGTAA | |
| | CTGTCAGACCAAGTTTACTCATATATACTTTAGATTGATTTAAAAC | |
| | TTCATTTTTAATTTAAAAGGATCTAGGTGAAGATCCTTTTTGATAAT | |
| | CTCATGACCAAAATCCCTTAACGTGAGTTTTCGTTCCACTGAGCGT | |
| | CAGACCCCGTAGAAAAGATCAAAGGATCTTCTTGAGATCCTTTTTT | |
| | TCTGCGCGTAATCTGCTGCTTGCAAACAAAAAAACCACCGCTACCA | |
| | GCGGTGGTTTGTTTGCCGGATCAAGAGCTACCAACTCTTTTTCCGA | |
| | AGGTAACTGGCTTCAGCAGAGCGCAGATACCAAATACTGTTCTTCT | |
| | AGTGTAGCCGTAGTTAGGCCACCACTTCAAGAACTCTGTAGCACCG | |
| | CCTACATACCTCGCTCTGCTAATCCTGTTACCAGTGGCTGCTGCCA | |
| | GTGGCGATAAGTCGTGTCTTACCGGGTTGGACTCAAGACGATAGTT | |
| | ACCGGATAAGGCGCAGCGGTCGGGCTGAACGGGGGGTTCGTGCAC | |
| | ACAGCCCAGCTTGGAGCGAACGACCTACACCGAACTGAGATACCT | |
| | ACAGCGTGAGCTATGAGAAAGCGCCACGCTTCCCGAAGGGAGAAA | |
| | GGCGGACAGGTATCCGGTAAGCGGCAGGGTCGGAACAGGAGAGC | |
| | GCACGAGGGAGCTTCCAGGGGGAAACGCCTGGTATCTTTATAGTC | |
| | CTGTCGGGTTTCGCCACCTCTGACTTGAGCGTCGATTTTTGTGATGC | |
| | TCGTCAGGGGGGCGGAGCCTATGGAAAAACGCCAGCAACGCGGCC | |
| | TTTTTACGGTTCCTGGCCTTTTGCTGGCCTTTTGCTCACATGTTCTTT | |
| | CCTGCGTTATCCCCTGATTCTGTGGATAACCGTATTACCGCCTTTGA | |
| | GTGAGCTGATACCGCTCGCCGCAGCCGAACGACCGAGCGCAGCGA | |
| | GTCAGTGAGCGAGGAAGCGGAAGAGCGCCCAATACGCAAACCGC | |
| | CTCTCCCCGCGCGTTGGCCGATTCATTAATGCAGCAAGCTCATGGC | |
| | TGACTAATTTTTTTTATTTATGCAGAGGCCGAGGCCGCCTCGGCCT | |
| | CTGAGCTATTCCAGAAGTAGTGAGGAGGCTTTTTTGGAGGCCTAGG | |
| | CTTTTGCAAAAAGCTCCCCGTGGCACGACAGGTTTCCCGACTGGAA | |
| | AGCGGGCAGTGAGCGCAACGCAATTAATGTGAGTTAGCTCACTCA | |
| | TTAGGCACCCCAGGCTTTACACTTTATGCTTCCGGCTCGTATGTTGT | |
| | GTGGAATTGTGAGCGGATAACAATTTCACACAGGAAACAGCTATG | |
| | ACATGATTACGAATTTCACAAATAAAGCATTTTTTTCACTGCATTC | |
| | TAGTTGTGGTTTGTCCAAACTCATCAATGTATCTTATCATGTCTGGA | |
| | TCAACTGGATAACTCAAGCTAACCAAAATCATCCCAAACTTCCCAC | |
| | CCCATACCCTATTACCACTGCCAATTACCTGTGGTTTCATTTACTCT | |
| | AAACCTGTGATTCCTCTGAATTATTTTCATTTTAAAGAAATTGTATT | |
| | TGTTAAATATGTACTACAAACTTAGTAGT | |
| 205 | TGGAAGGGCTAATTCACTCCCAAAGAAGACAAGATATCCTTGATC | pMA2 N-term |
| | TGTGGATCTACCACACACAAGGCTACTTCCCTGATTAGCAGAACTA | GAG-T2A |
| | CACACCAGGGCCAGGGGTCAGATATCCACTGACCTTTGGATGGTG | |
| | CTACAAGCTAGTACCAGTTGAGCCAGATAAGGTAGAAGAGGCCAA | |
| | TAAAGGAGAGAACACCAGCTTGTTACACCCTGTGAGCCTGCATGG | |
| | GATGGATGACCCGGAGAGAGAAGTGTTAGAGTGGAGGTTTGACAG | |
| | CCGCCTAGCATTTCATCACGTGGCCCGAGAGCTGCATCCGGAGTAC | |
| | TTCAAGAACTGCTGATATCGAGCTTGCTACAAGGGACTTTCCGCTG | |
| | GGGACTTTCCAGGGAGGCGTGGCCTGGGCGGGACTGGGGAGTGGC | |
| | GAGCCCTCAGATCCTGCATATAAGCAGCTGCTTTTTGCCTGTACTG | |
| | GGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGA | |
| | GATCCCTCAGACCCTTTTAGTCAGTGTGGAAAATCTCTAGCAGTGG | |
| | CGCCCGAACAGGGACTTGAAAGCGAAAGGGAAACCAGAGGAGCT | |
| | CTCTCGACGCAGGACTCGGCTTGCTGAAGCGCGCACGGCAAGAGG | |
| | CGAGGGGCGGCGACTGGTGAGTACGCCAAAAATTTTGACTAGCGG | |
| | AGGCTAGAAGGAGAGAGATGGGTGCGAGAGCGTCGGTATTAAGC | |
| | GGGGGAGAATTAGATAAATGGGAAAAAATTCGGTTAAGGCCAGG | |
| | GGGAAAGAAACAATATAAACTAAAACATATAGTATGGGCAAGCA | |
| | GGGAGCTAGAACGATTCGCAGTTAATCCTGGCCTTTTAGAGACATC | |
| | AGAAGGCTGTAGACAAATACTGGGACAGCTACAACCATCCCTTCA | |
| | GACAGGATCAGAAGAACTTAGATCATTATATAATACAATAGCAGT | |
| | CCTCTATTGTGTGCATCAAAGGATAGATGTAAAAGACACCAAGGA | |
| | AGCCTTAGATAAGATAGAGGAAGAGCAAAACAAAAGTAAGAAAA | |
| | AGGCACAGCAAGCAGGTAGTGGCGAGGGCAGAGGAAGTCTTCTAA | |
| | CATGCGGTGACGTGGAGGAGAATCCCGGCCCTTGAGGATCCTAAT | |
| | CAACCTCTGGATTACAAAATTTGTGAAAGATTGACTGGTATTCTTA | |
| | ACTATGTTGCTCCTTTTACGCTATGTGGATACGCTGCTTTAATGCCT | |
| | TTGTATCATGCTATTGCTTCCCGTATGGCTTTCATTTTCTCCTCCTTG | |
| | TATAAATCCTGGTTGCTGTCTCTTTATGAGGAGTTGTGGCCCGTTGT | |
| | CAGGCAACGTGGCGTGGTGTGCACTGTGTTTGCTGACGCAACCCCC | |
| | ACTGGTTGGGGCATTGCCACCACCTGTCAGCTCCTTTCCGGGACTT | |
| | TCGCTTTCCCCCTCCCTATTGCCACGGCGGAACTCATCGCCGCCTG | |
| | CCTTGCCCGCTGCTGGACAGGGGCTCGGCTGTTGGGCACTGACAAT | |
| | TCCGTGGTGTTGTCGGGGAAATCATCGTCCTTTCCTTGGCTGCTCG | |
| | CCTGTGTTGCCACCTGGATTCTGCGCGGGACGTCCTTCTGCTACGT | |
| | CCCTTCGGCCCTCAATCCAGCGGACCTTCCTTCCCGCGGCCTGCTG | |
| | CCGGCTCTGCGGCCTCTTCCGCGTCTTCGCCTTCGCCCTCAGACGA | |
| | GTCGGATCTCCCTTTGGGCCGCCTCCCCGCCTGAGATCCTTTAAGA | |
| | CCAATGACTTACAAGGCAGCTGTAGATCTTAGCCACTTTTTAAAAG | |
| | AAAAGGGGGGACTGGAAGGGCTAATTCACTCCCAACGAAGACAA | |
| | GATCTGCTTTTTGCTTGTACTGGGTCTCTCTGGTTAGACCAGATCTG | |
| | AGCCTGGGAGCTCTCTGGCTAACTAGGGAACCCACTGCTTAAGCCT | |
| | CAATAAAGCTTGCCTTGAGTGCTTCAAGTAGTGTGTGCCCGTCTGT | |
| | TGTGTGACTCTGGTAACTAGAGATCCCTCAGACCCTTTTAGTCAGT | |
| | GTGGAAAATCTCTAGCAGTAGTAGTTCATGTCATCTTATTATTCAG | |
| | TATTTATAACTTGCAAAGAAATGAATATCAGAGAGTGAGAGGCCC | |
| | GGGTTAATTAAGGAAAGGGCTAGATCATTCTTGAAGACGAAAGGG | |
| | CCTCGTGATACGCCTATTTTTATAGGTTAATGTCATGATAATAATG | |
| | GTTTCTTAGACGTCAGGTGGCACTTTTCGGGGAAATGTGCGCGGAA | |
| | CCCCTATTTGTTTATTTTTCTAAATACATTCAAATATGTATCCGCTC | |
| | ATGAGACAATAACCCTGATAAATGCTTCAATAATATTGAAAAAGG | |
| | AAGAGTATGAGTATTCAACATTTCCGTGTCGCCCTTATTCCCTTTTT | |
| | TGCGGCATTTTGCCTTCCTGTTTTTGCTCACCCAGAAACGCTGGTG | |
| | AAAGTAAAAGATGCTGAAGATCAGTTGGGTGCACGAGTGGGTTAC | |
| | ATCGAACTGGATCTCAACAGCGGTAAGATCCTTGAGAGTTTTCGCC | |
| | CCGAAGAACGTTTTCCAATGATGAGCACTTTTAAAGTTCTGCTATG | |
| | TGGCGCGGTATTATCCCGTGTTGACGCCGGGCAAGAGCAACTCGG | |
| | TCGCCGCATACACTATTCTCAGAATGACTTGGTTGAGTACTCACCA | |
| | GTCACAGAAAAGCATCTTACGGATGGCATGACAGTAAGAGAATTA | |
| | TGCAGTGCTGCCATAACCATGAGTGATAACACTGCGGCCAACTTAC | |
| | TTCTGACAACGATCGGAGGACCGAAGGAGCTAACCGCTTTTTTGCA | |
| | CAACATGGGGGATCATGTAACTCGCCTTGATCGTTGGGAACCGGA | |
| | GCTGAATGAAGCCATACCAAACGACGAGCGTGACACCACGATGCC | |
| | TGTAGCAATGGCAACAACGTTGCGCAAACTATTAACTGGCGAACT | |
| | ACTTACTCTAGCTTCCCGGCAACAATTAATAGACTGGATGGAGGCG | |
| | GATAAAGTTGCAGGACCACTTCTGCGCTCGGCCCTTCCGGCTGGCT | |
| | GGTTTATTGCTGATAAATCTGGAGCCGGTGAGCGTGGGTCTCGCGG | |
| | TATCATTGCAGCACTGGGGCCAGATGGTAAGCCCTCCCGTATCGTA | |
| | GTTATCTACACGACGGGGAGTCAGGCAACTATGGATGAACGAAAT | |
| | AGACAGATCGCTGAGATAGGTGCCTCACTGATTAAGCATTGGTAA | |
| | CTGTCAGACCAAGTTTACTCATATATACTTTAGATTGATTTAAAAC | |
| | TTCATTTTTAATTTAAAAGGATCTAGGTGAAGATCCTTTTTGATAAT | |
| | CTCATGACCAAAATCCCTTAACGTGAGTTTTCGTTCCACTGAGCGT | |
| | CAGACCCCGTAGAAAAGATCAAAGGATCTTCTTGAGATCCTTTTTT | |
| | TCTGCGCGTAATCTGCTGCTTGCAAACAAAAAAACCACCGCTACCA | |
| | GCGGTGGTTTGTTTGCCGGATCAAGAGCTACCAACTCTTTTTCCGA | |
| | AGGTAACTGGCTTCAGCAGAGCGCAGATACCAAATACTGTTCTTCT | |
| | AGTGTAGCCGTAGTTAGGCCACCACTTCAAGAACTCTGTAGCACCG | |
| | CCTACATACCTCGCTCTGCTAATCCTGTTACCAGTGGCTGCTGCCA | |
| | GTGGCGATAAGTCGTGTCTTACCGGGTTGGACTCAAGACGATAGTT | |
| | ACCGGATAAGGCGCAGCGGTCGGGCTGAACGGGGGGTTCGTGCAC | |
| | ACAGCCCAGCTTGGAGCGAACGACCTACACCGAACTGAGATACCT | |
| | ACAGCGTGAGCTATGAGAAAGCGCCACGCTTCCCGAAGGGAGAAA | |
| | GGCGGACAGGTATCCGGTAAGCGGCAGGGTCGGAACAGGAGAGC | |
| | GCACGAGGGAGCTTCCAGGGGGAAACGCCTGGTATCTTTATAGTC | |
| | CTGTCGGGTTTCGCCACCTCTGACTTGAGCGTCGATTTTTGTGATGC | |
| | TCGTCAGGGGGGCGGAGCCTATGGAAAAACGCCAGCAACGCGGCC | |
| | TTTTTACGGTTCCTGGCCTTTTGCTGGCCTTTTGCTCACATGTTCTTT | |
| | CCTGCGTTATCCCCTGATTCTGTGGATAACCGTATTACCGCCTTTGA | |
| | GTGAGCTGATACCGCTCGCCGCAGCCGAACGACCGAGCGCAGCGA | |
| | GTCAGTGAGCGAGGAAGCGGAAGAGCGCCCAATACGCAAACCGC | |
| | CTCTCCCCGCGCGTTGGCCGATTCATTAATGCAGCAAGCTCATGGC | |
| | TGACTAATTTTTTTTATTTATGCAGAGGCCGAGGCCGCCTCGGCCT | |
| | CTGAGCTATTCCAGAAGTAGTGAGGAGGCTTTTTTGGAGGCCTAGG | |
| | CTTTTGCAAAAAGCTCCCCGTGGCACGACAGGTTTCCCGACTGGAA | |
| | AGCGGGCAGTGAGCGCAACGCAATTAATGTGAGTTAGCTCACTCA | |
| | TTAGGCACCCCAGGCTTTACACTTTATGCTTCCGGCTCGTATGTTGT | |
| | GTGGAATTGTGAGCGGATAACAATTTCACACAGGAAACAGCTATG | |
| | ACATGATTACGAATTTCACAAATAAAGCATTTTTTTCACTGCATTC | |
| | TAGTTGTGGTTTGTCCAAACTCATCAATGTATCTTATCATGTCTGGA | |
| | TCAACTGGATAACTCAAGCTAACCAAAATCATCCCAAACTTCCCAC | |
| | CCCATACCCTATTACCACTGCCAATTACCTGTGGTTTCATTTACTCT | |
| | AAACCTGTGATTCCTCTGAATTATTTTCATTTTAAAGAAATTGTATT | |
| | TGTTAAATATGTACTACAAACTTAGTAGT | |
| 206 | AGCTTGGCCCATTGCATACGTTGTATCCATATCATAATATGTACAT | MS2_{cp} mRNA; |
| | TTATATTGGCTCATGTCCAACATTACCGCCATGTTGACATTGATTAT | HIV MA (N- |
| | TGACTAGTTATTAATAGTAATCAATTACGGGGTCATTAGTTCATAG | term) |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCAGGATCCAACCTACA | |
| | AACGGGTGGAGGATCACCCCACCCGACACTTCACAATCAAGGGGT | |
| | ACAATACACAAGGGTGGAGGAACACCCCACCCTCCAGACACATTA | |
| | CACAGAAATCCAATCAAACAGAAGCACCATCAGGGCTTCTGCTAC | |
| | CAAATTTATCTCAAAAAACTACAACAAGGAATCACCATCAGGGAT | |
| | TCCCTGTGCAATATACGTCAAACGAGGGCCACGACGGGAGGACGA | |
| | TCACGCCTCCCGAATATCGGCATGTCTGGCTTTCGAATTCAGTGCG | |
| | TGGAGCATCAGCCCACGCAGCCAATCAGAGTCGAATACAAGTCGA | |
| | CTTTCGCGAAGAGCATCAGCCTTCGCGCCATTCTTACACAAACCAC | |
| | ACTCTCCCCTACAGGAACAGCATCAGCGTTCCTGCCCAGTACCCAA | |
| | CTCAAGAAAATTTATGTCCCCATGCAGCATCAGCGCATGGGCCCCA | |
| | AGAATACATCCCCAACAAAATCACATCCGAGCACCAACAGGGCTC | |
| | GGAGTGTTGTTTCTTGTCCAACTGGACAAACCCTCCATGGACCATC | |
| | AGGCCATGGACTCTCACCAACAAGACAAAAACTACTCTTCTCGAA | |
| | GCAGCATCAGCGCTTCGAAACACTCGACCTCGAGGGCCCAGATCT | |
| | AATTCACCCCACCAGTGCAGGCTGCCTATCAGAAAGTGGTGGCTG | |
| | GTGTGGCTAATGCCCTGGCCCACAAGTATCACTAAGCTCGCTTTCT | |
| | TGCTGTCCAATTTCTATTAAAGGTTCCTTTGTTCCCTAAGTCCAACT | |
| | ACTAAACTGGGGGATATTATGAAGGGCCTTGAGCATCTGGATTCTG | |
| | CCTAATAAAAAACATTTATTTTCATTGCAATGATGTATTTAAATTAT | |
| | TTCTGAATATTTTACTAAAAAGGGAATGTGGGAGGTCAGTGCATTT | |
| | AAAACATAAAGAAATGAAGAGCTAGTTCAAACCTTGGGAAAATAC | |
| | ACTATATCTTAAACTCCATGAAAGAAGGTGAGGCTGCAAACAGCT | |
| | AATGCACATTGGCAACAGCCCCTGATGCCTATGCCTTATTCATCCC | |
| | TCAGAAAAGGATTCAAGTAGAGGCTTGATTTGGAGGTTAAAGTTTT | |
| | GCTATGCTGTATTTTACATTACTTATTGTTTTAGCTGTCCTCATGAA | |
| | TGTCTTTTCACTACCCATTTGCTTATCCTGCATCTCTCAGCCTTGAC | |
| | TCCACTCAGTTCTCTTGCTTAGAGATACCACCTTTCCCCTGAAGTGT | |
| | TCCTTCCATGTTTTACGGCGAGATGGTTTCTCCTCGCCTGGCCACTC | |
| | AGCCTTAGTTGTCTCTGTTGTCTTATAGAGGTCTACTTGAAGAAGG | |
| | AAAAACAGGGGGCATGGTTTGACTGTCCTGTGAGCCCTTCTTCCCT | |
| | GCCTCCCCCACTCACAGTGACCCGGAATCCCTCGACATGGCAGTCT | |
| | AGATCATTCTTGAAGACGAAAGGGCCTCGTGATACGCCTATTTTTA | |
| | TAGGTTAATGTCATGATAATAATGGTTTCTTAGACGTCAGGTGGCA | |
| | CTTTTCGGGGAAATGTGCGCGGAACCCCTATTTGTTTATTTTTCTAA | |
| | ATACATTCAAATATGTATCCGCTCATGAGACAATAACCCTGATAAA | |
| | TGCTTCAATAATATTGAAAAAGGAAGAGTATGAGTATTCAACATTT | |
| | CCGTGTCGCCCTTATTCCCTTTTTTGCGGCATTTTGCCTTCCTGTTTT | |
| | TGCTCACCCAGAAACGCTGGTGAAAGTAAAAGATGCTGAAGATCA | |
| | GTTGGGTGCACGAGTGGGTTACATCGAACTGGATCTCAACAGCGG | |
| | TAAGATCCTTGAGAGTTTTCGCCCCGAAGAACGTTTTCCAATGATG | |
| | AGCACTTTTAAAGTTCTGCTATGTGGCGCGGTATTATCCCGTATTG | |
| | ACGCCGGGCAAGAGCAACTCGGTCGCCGCATACACTATTCTCAGA | |
| | ATGACTTGGTTGAGTACTCACCAGTCACAGAAAAGCATCTTACGG | |
| | ATGGCATGACAGTAAGAGAATTATGCAGTGCTGCCATAACCATGA | |
| | GTGATAACACTGCGGCCAACTTACTTCTGACAACGATCGGAGGAC | |
| | CGAAGGAGCTAACCGCTTTTTTGCACAACATGGGGGATCATGTAA | |
| | CTCGCCTTGATCGTTGGGAACCGGAGCTGAATGAAGCCATACCAA | |
| | ACGACGAGCGTGACACCACGATGCCTGTAGCAATGGCAACAACGT | |
| | TGCGCAAACTATTAACTGGCGAACTACTTACTCTAGCTTCCCGGCA | |
| | ACAATTAATAGACTGGATGGAGGCGGATAAAGTTGCAGGACCACT | |
| | TCTGCGCTCGGCCCTTCCGGCTGGCTGGTTTATTGCTGATAAATCT | |
| | GGAGCCGGTGAGCGTGGGTCTCGCGGTATCATTGCAGCACTGGGG | |
| | CCAGATGGTAAGCCCTCCCGTATCGTAGTTATCTACACGACGGGGA | |
| | GTCAGGCAACTATGGATGAACGAAATAGACAGATCGCTGAGATAG | |
| | GTGCCTCACTGATTAAGCATTGGTAACTGTCAGACCAAGTTTACTC | |
| | ATATATACTTTAGATTGATTTAAAACTTCATTTTTAATTTAAAAGGA | |
| | TCTAGGTGAAGATCCTTTTTGATAATCTCATGACCAAAATCCCTTA | |
| | ACGTGAGTTTTCGTTCCACTGAGCGTCAGACCCCGTAGAAAAGATC | |
| | AAAGGATCTTCTTGAGATCCTTTTTTTCTGCGCGTAATCTGCTGCTT | |
| | GCAAACAAAAAAACCACCGCTACCAGCGGTGGTTTGTTTGCCGGA | |
| | TCAAGAGCTACCAACTCTTTTTCCGAAGGTAACTGGCTTCAGCAGA | |
| | GCGCAGATACCAAATACTGTTCTTCTAGTGTAGCCGTAGTTAGGCC | |
| | ACCACTTCAAGAACTCTGTAGCACCGCCTACATACCTCGCTCTGCT | |
| | AATCCTGTTACCAGTGGCTGCTGCCAGTGGCGATAAGTCGTGTCTT | |
| | ACCGGGTTGGACTCAAGACGATAGTTACCGGATAAGGCGCAGCGG | |
| | TCGGGCTGAACGGGGGGTTCGTGCACACAGCCCAGCTTGGAGCGA | |
| | ACGACCTACACCGAACTGAGATACCTACAGCGTGAGCTATGAGAA | |
| | AGCGCCACGCTTCCCGAAGGGAGAAAGGCGGACAGGTATCCGGTA | |
| | AGCGGCAGGGTCGGAACAGGAGAGCGCACGAGGGAGCTTCCAGG | |
| | GGGAAACGCCTGGTATCTTTATAGTCCTGTCGGGTTTCGCCACCTC | |
| | TGACTTGAGCGTCGATTTTTGTGATGCTCGTCAGGGGGGCGGAGCC | |
| | TATGGAAAAACGCCAGCAACGGATGCGCCGCGTGCGGCTGCTGGA | |
| | GATGGCGGACGCGATGGATATGTTCTGCCAAGGGTTGGTTTGCGC | |
| | ATTCACAGTTCTCCGCAAGAATTGATTGGCTCCAATTCTTGGAGTG | |
| | GTGAATCCGTTAGCGAGGTGCCGCCGGCTTCCATTCAGGTCGAGGT | |
| | GGCCCGGCTCCATGCACCGCGACGCAACGCGGGGAGGCAGACAAG | |
| | GTATAGGGCGGCGCCTACAATCCATGCCAACCCGTTCCATGTGCTC | |
| | GCCGAGGCGGCATAAATCCCCGTGACGATCAGCGGTCCAATGATC | |
| | GAAGTTAGGCTGGTAAGAGCCGCGAGCGATCCTTGAAGCTGTCCC | |
| | TGATGGTCGTCATCTACCTGCCTGGACAGCATGGCCTGCAACGCGG | |
| | GCATCCCGATGCCGCCGGAAGCGAGAAGAATCATAATGGGGAAGG | |
| | CCATCCAGCCTCGCGTCGGGGAGCTTTTTGCAAAAGCCTAGGCCTC | |
| | CAAAAAAGCCTCCTCACTACTTCTGGAATAGCTCAGAGGCCGAGG | |
| | CGGCCTCGGCCTCTGCATAAATAAAAAAAATTAGTCAGCCATG | |
| 207 | AGCTTGGCCCATTGCATACGTTGTATCCATATCATAATATGTACAT | MA; |
| | TTATATTGGCTCATGTCCAACATTACCGCCATGTTGACATTGATTAT | HIV MA (N- |
| | TGACTAGTTATTAATAGTAATCAATTACGGGGTCATTAGTTCATAG | term) |
| | CCCATATATGGAGTTCCGCGTTACATAACTTACGGTAAATGGCCCG | |
| | CCTGGCTGACCGCCCAACGACCCCCGCCCATTGACGTCAATAATGA | |
| | CGTATGTTCCCATAGTAACGCCAATAGGGACTTTCCATTGACGTCA | |
| | ATGGGTGGAGTATTTACGGTAAACTGCCCACTTGGCAGTACATCAA | |
| | GTGTATCATATGCCAAGTACGCCCCCTATTGACGTCAATGACGGTA | |
| | AATGGCCCGCCTGGCATTATGCCCAGTACATGACCTTATGGGACTT | |
| | TCCTACTTGGCAGTACATCTACGTATTAGTCATCGCTATTACCATG | |
| | GTGATGCGGTTTTGGCAGTACATCAATGGGCGTGGATAGCGGTTTG | |
| | ACTCACGGGGATTTCCAAGTCTCCACCCCATTGACGTCAATGGGAG | |
| | TTTGTTTTGGCACCAAAATCAACGGGACTTTCCAAAATGTCGTAAC | |
| | AACTCCGCCCCATTGACGCAAATGGGCGGTAGGCGTGTACGGTGG | |
| | GAGGTCTATATAAGCAGAGCTCGTTTAGTGAACCGTCAGATCGCCT | |
| | GGAGACGCCATCCACGCTGTTTTGACCTCCATAGAAGACACCGGG | |
| | ACCGATCCAGCCTCCCCTCGAAGCTGATCCTGAGAACTTCAGGGTG | |
| | AGTCTATGGGACCCTTGATGTTTTCTTTCCCCTTCTTTTCTATGGTT | |
| | AAGTTCATGTCATAGGAAGGGGAGAAGTAACAGGGTACACATATT | |
| | GACCAAATCAGGGTAATTTTGCATTTGTAATTTTAAAAAATGCTTT | |
| | CTTCTTTTAATATACTTTTTTGTTTATCTTATTTCTAATACTTTCCCT | |
| | AATCTCTTTCTTTCAGGGCAATAATGATACAATGTATCATGCCTCTT | |
| | TGCACCATTCTAAAGAATAACAGTGATAATTTCTGGGTTAAGGCAA | |
| | TAGCAATATTTCTGCATATAAATATTTCTGCATATAAATTGTAACT | |
| | GATGTAAGAGGTTTCATATTGCTAATAGCAGCTACAATCCAGCTAC | |
| | CATTCTGCTTTTATTTTATGGTTGGGATAAGGCTGGATTATTCTGAG | |
| | TCCAAGCTAGGCCCTTTTGCTAATCATGTTCATACCTCTTATCTTCC | |
| | TCCCACAGCTCCTGGGCAACGTGCTGGTCTGTGTGCTGGCCCATCA | |
| | CTTTGGCAAAGAATTCCGCGGGCGGCCGCCACCATGGGCGCCCGC | |
| | GCCTCCGTGCTGTCCGGCGGCGAGCTGGACAAGTGGGAGAAGATC | |
| | CGCCTGCGCCCCGGCGGCAAGAAGCAGTACAAGCTGAAGCACATC | |
| | GTGTGGGCCTCCCGCGAGCTGGAGCGCTTCGCCGTGAACCCCGGC | |
| | CTGCTGGAAACCTCCGAGGGCTGCCGCCAGATCCTGGGCCAGCTG | |
| | CAGCCCTCCCTGCAAACCGGCTCCGAGGAGCTGCGCTCCCTGTACA | |
| | ACACCATCGCCGTGCTGTACTGCGTGCACCAGCGCATCGACGTGA | |
| | AGGACACCAAGGAGGCCCTGGACAAGATCGAGGAGGAGCAGAAC | |
| | AAGTCCAAGAAGAAGGCCCAGCAGGCCGCCGCCGACACCGGCAA | |
| | CAACTCCCAGGTGTCCCAGAACTACCCCATCGTGCAGAGGATCCA | |
| | AGCTTATCGATACCGTCGACCTCGAGGGCCCAGATCTAATTCACCC | |
| | CACCAGTGCAGGCTGCCTATCAGAAAGTGGTGGCTGGTGTGGCTA | |
| | ATGCCCTGGCCCACAAGTATCACTAAGCTCGCTTTCTTGCTGTCCA | |
| | ATTTCTATTAAAGGTTCCTTTGTTCCCTAAGTCCAACTACTAAACTG | |
| | GGGGATATTATGAAGGGCCTTGAGCATCTGGATTCTGCCTAATAAA | |
| | AAACATTTATTTTCATTGCAATGATGTATTTAAATTATTTCTGAATA | |
| | TTTTACTAAAAAGGGAATGTGGGAGGTCAGTGCATTTAAAACATA | |
| | AAGAAATGAAGAGCTAGTTCAAACCTTGGGAAAATACACTATATC | |
| | TTAAACTCCATGAAAGAAGGTGAGGCTGCAAACAGCTAATGCACA | |
| | TTGGCAACAGCCCCTGATGCCTATGCCTTATTCATCCCTCAGAAAA | |
| | GGATTCAAGTAGAGGCTTGATTTGGAGGTTAAAGTTTTGCTATGCT | |
| | GTATTTTACATTACTTATTGTTTTAGCTGTCCTCATGAATGTCTTTTC | |
| | ACTACCCATTTGCTTATCCTGCATCTCTCAGCCTTGACTCCACTCAG | |
| | TTCTCTTGCTTAGAGATACCACCTTTCCCCTGAAGTGTTCCTTCCAT | |
| | GTTTTACGGCGAGATGGTTTCTCCTCGCCTGGCCACTCAGCCTTAG | |
| | TTGTCTCTGTTGTCTTATAGAGGTCTACTTGAAGAAGGAAAAACAG | |
| | GGGGCATGGTTTGACTGTCCTGTGAGCCCTTCTTCCCTGCCTCCCCC | |
| | ACTCACAGTGACCCGGAATCCCTCGACATGGCAGTCTAGATCATTC | |
| | TTGAAGACGAAAGGGCCTCGTGATACGCCTATTTTTATAGGTTAAT | |
| | GTCATGATAATAATGGTTTCTTAGACGTCAGGTGGCACTTTTCGGG | |
| | GAAATGTGCGCGGAACCCCTATTTGTTTATTTTTCTAAATACATTC | |
| | AAATATGTATCCGCTCATGAGACAATAACCCTGATAAATGCTTCAA | |
| | TAATATTGAAAAAGGAAGAGTATGAGTATTCAACATTTCCGTGTCG | |
| | CCCTTATTCCCTTTTTTGCGGCATTTTGCCTTCCTGTTTTTGCTCACC | |
| | CAGAAACGCTGGTGAAAGTAAAAGATGCTGAAGATCAGTTGGGTG | |
| | CACGAGTGGGTTACATCGAACTGGATCTCAACAGCGGTAAGATCC | |
| | TTGAGAGTTTTCGCCCCGAAGAACGTTTTCCAATGATGAGCACTTT | |
| | TAAAGTTCTGCTATGTGGCGCGGTATTATCCCGTATTGACGCCGGG | |
| | CAAGAGCAACTCGGTCGCCGCATACACTATTCTCAGAATGACTTGG | |
| | TTGAGTACTCACCAGTCACAGAAAAGCATCTTACGGATGGCATGA | |
| | CAGTAAGAGAATTATGCAGTGCTGCCATAACCATGAGTGATAACA | |
| | CTGCGGCCAACTTACTTCTGACAACGATCGGAGGACCGAAGGAGC | |
| | TAACCGCTTTTTTGCACAACATGGGGGATCATGTAACTCGCCTTGA | |
| | TCGTTGGGAACCGGAGCTGAATGAAGCCATACCAAACGACGAGCG | |
| | TGACACCACGATGCCTGTAGCAATGGCAACAACGTTGCGCAAACT | |
| | ATTAACTGGCGAACTACTTACTCTAGCTTCCCGGCAACAATTAATA | |
| | GACTGGATGGAGGCGGATAAAGTTGCAGGACCACTTCTGCGCTCG | |
| | GCCCTTCCGGCTGGCTGGTTTATTGCTGATAAATCTGGAGCCGGTG | |
| | AGCGTGGGTCTCGCGGTATCATTGCAGCACTGGGGCCAGATGGTA | |
| | AGCCCTCCCGTATCGTAGTTATCTACACGACGGGGAGTCAGGCAA | |
| | CTATGGATGAACGAAATAGACAGATCGCTGAGATAGGTGCCTCAC | |
| | TGATTAAGCATTGGTAACTGTCAGACCAAGTTTACTCATATATACT | |
| | TTAGATTGATTTAAAACTTCATTTTTAATTTAAAAGGATCTAGGTG | |
| | AAGATCCTTTTTGATAATCTCATGACCAAAATCCCTTAACGTGAGT | |
| | TTTCGTTCCACTGAGCGTCAGACCCCGTAGAAAAGATCAAAGGAT | |
| | CTTCTTGAGATCCTTTTTTTCTGCGCGTAATCTGCTGCTTGCAAACA | |
| | AAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTT | |
| | CAAGAACTCTGTAGCACCGCCTACATACCTCGCTCTGCTAATCCTG | |
| | TTACCAGTGGCTGCTGCCAGTGGCGATAAGTCGTGTCTTACCGGGT | |
| | TGGACTCAAGACGATAGTTACCGGATAAGGCGCAGCGGTCGGGCT | |
| | GAACGGGGGGTTCGTGCACACAGCCCAGCTTGGAGCGAACGACCT | |
| | ACACCGAACTGAGATACCTACAGCGTGAGCTATGAGAAAGCGCCA | |
| | CGCTTCCCGAAGGGAGAAAGGCGGACAGGTATCCGGTAAGCGGCA | |
| | GGGTCGGAACAGGAGAGCGCACGAGGGAGCTTCCAGGGGGAAAC | |
| | GCCTGGTATCTTTATAGTCCTGTCGGGTTTCGCCACCTCTGACTTGA | |
| | GCGTCGATTTTTGTGATGCTCGTCAGGGGGGCGGAGCCTATGGAA | |
| | AAACGCCAGCAACGGAGATGCGCCGCGTGCGGCTGCTGGAGATGG | |
| | CGGACGCGATGGATATGTTCTGCCAAGGGTTGGTTTGCGCATTCAC | |
| | AGTTCTCCGCAAGAATTGATTGGCTCCAATTCTTGGAGTGGTGAAT | |
| | CCGTTAGCGAGGTGCCGCCGGCTTCCATTCAGGTCGAGGTGGCCCG | |
| | GCTCCATGCACCGCGACGCAACGCGGGGAGGCAGACAAGGTATAG | |
| | GGCGGCGCCTACAATCCATGCCAACCCGTTCCATGTGCTCGCCGAG | |
| | GCGGCATAAATCGCCGTGACGATCAGCGGTCCAATGATCGAAGTT | |
| | AGGCTGGTAAGAGCCGCGAGCGATCCTTGAAGCTGTCCCTGATGG | |
| | TCGTCATCTACCTGCCTGGACAGCATGGCCTGCAACGCGGGCATCC | |
| | CGATGCCGCCGGAAGCGAGAAGAATCATAATGGGGAAGGCCATCC | |
| | AGCCTCGCGTCGGGGAGCTTTTTGCAAAAGCCTAGGCCTCCAAAA | |
| | AAGCCTCCTCACTACTTCTGGAATAGCTCAGAGGCCGAGGCGGCCT | |
| | CGGCCTCTGCATAAATAAAAAAAATTAGTCAGCCATG | |
| 208 | N₁R₂N₃D₄S₅AS₆S₇ANCAS₇′S₆′S₅′N₄′N₃′Y₂′N₁′ | MS2 stem loop |
| | | configuration, |
| | | Nis any |
| | | nucleotide, S |
| | | represents C or |
| | | G; D |
| | | represents A, |
| | | G, or U; R |
| | | represents A or |
| | | G; Y |
| | | represents C or |
| | | U. Apostrophe |
| | | indicates |
| | | nucleotide is |
| | | complementar |
| | | y to the |
| | | nucleotide |
| | | with same |
| | | subscript |
| | | number |

What is claimed:

1. A lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a RNA sequence encoding a gag protein or portion thereof comprising at least a gag start codon; a RNA sequence encoding a heterologous protein that is operably linked to the RNA sequence encoding a gag protein or portion thereof; and a poly-A tail,
wherein each of the R element of the 5′ LTR, the U5 element of the 5′ LTR, and the RNA sequence encoding a gag protein or portion thereof is retroviral.

2. A lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a gag 5′ untranslated region (UTR) or portion thereof comprising at least three nucleotides; a RNA sequence encoding a heterologous protein that is operably linked to the gag 5′ UTR or a portion thereof; and a poly-A tail,
wherein each of the R element of the 5′ LTR, the U5 element of the 5′ LTR, and the gag 5′ UTR or portion thereof is retroviral.

3. The lipid particle of claim 1 or claim 2, wherein the RNA comprises a retroviral packaging sequence that is 3′ to the 5′ LTR.

4. A lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a retroviral packaging sequence; a gag start codon; a RNA sequence encoding a heterologous protein; and a poly-A tail,
wherein each of the R element of the 5′ LTR, the U5 element of the 5′ LTR, and the gag start codon is retroviral.

5. The lipid particle of any of claims 1-4, further comprising a U3 element of a 5′ LTR.

6. The lipid particle of any of claims 1-5, wherein the RNA comprises a polyadenylation site.

7. The lipid particle of claim 6, wherein the RNA comprises a 3′ long terminal repeat (3′ LTR), and the polyadenylation site is located within the 3′ LTR.

8. The lipid particle of any of claims 1-7, wherein the RNA comprises a mutated primer binding site (PBS).

9. The lipid particle of any of claims 3-8, wherein the retroviral packaging sequence is selected from the group comprising HIV psi, MLV psi, SNV E, or a portion of any thereof.

10. The lipid particle of any of claims 3-9, wherein the retroviral packaging sequence comprises stem-loop 1 (SL1) of HIV psi.

11. The lipid particle of any of claims 3-10, wherein the retroviral packaging sequence comprises stem-loop 2 (SL2) of HIV psi.

12. The lipid particle of any of claims 3-11, wherein the retroviral packaging sequence comprises stem-loop 3 (SL3) of HIV psi.

13. The lipid particle of any of claims 3-12, wherein the retroviral packaging sequence comprises stem-loop 4 (SL4) of HIV psi.

14. The lipid particle of any one of claims 3-13, wherein the retroviral packaging sequence is HIV psi.

15. The lipid particle of any one of claims 3-14, wherein the retroviral packaging sequence comprises a mutation in a major splice donor site.

16. The lipid particle of claim 15, wherein the major splice donor site is a major splice donor site contained in SL2 of HIV psi.

17. The lipid particle of claim 15 or claim 16, wherein the mutation is a mutation that inhibits splicing at the major splice donor site.

18. The lipid particle of any one of claims 15-17, wherein the mutation in the major splice donor site comprises a mutation that prevents splicing at the major splice donor site.

19. The lipid particle of any of claims 1 and 3-18, wherein the RNA comprises a retroviral sequence having at least about 80% sequence identity to the sequence of a retroviral genome that is about 20, 30, 40, 50, 60, 70, 80, 90, 100, 120, 140, 160, 180, 200, 220, 240, 260, 280, 300, 320, 340, 360, 380, or 400 nucleotides in length and comprises the gag start codon.

20. The lipid particle of any of claims 2, 3, and 5-18, wherein the RNA comprises a retroviral sequence having at least about 80% sequence identity to the sequence of a retroviral genome that is about 20, 30, 40, 50, 60, 70, 80, 90, 100, 120, 140, 160, 180, 200, 220, 240, 260, 280, 300, 320, 340, 360, 380, or 400 nucleotides in length and comprises a gag start codon.

21. The lipid particle of claim 19 or claim 20, wherein the retroviral sequence comprises between about 20-400, between about 40 and about 350, between about 60 and about 300, between about 80 and about 250, or between about 100 and about 200 nucleotides 5′ to the gag start codon.

22. The lipid particle of any of claims 19-21, wherein the retroviral sequence comprises between about 20 and about 400, between about 40 and about 350, between about 60 and about 300, between about 80 and about 250, or between about 100 and about 200 nucleotides 3′ to the gag start codon.

23. The lipid particle of any of claims 1-22, wherein the lumen comprises a capsid comprising a retroviral capsid protein enclosing the RNA.

24. The lipid particle of claim 23, wherein the retroviral capsid protein and the retroviral packaging sequence are capable of associating with each other, optionally wherein the retroviral capsid protein and the retroviral packaging sequence are from the same retroviral species.

25. The lipid particle of any of claims 1-24, wherein the lipid particle comprises a retroviral matrix protein.

26. The lipid particle of any of claims 1 and 3-25, further comprising a RNA sequence encoding a viral structural protein or a portion thereof, which is located between the gag start codon and the RNA sequence encoding a heterologous protein.

27. The lipid particle of any of claims 1 and 3-25, wherein the RNA does not comprise nucleotides between the gag start codon and the RNA sequence encoding a heterologous protein.

28. A lipid particle comprising a lipid bilayer enclosing a lumen and a ribonucleic acid (RNA), wherein the RNA comprises, from 5′ to 3′: a R element of a 5′ long terminal repeat (5′ LTR); a U5 element of a 5′ LTR; a RNA sequence encoding a viral structural protein or a portion thereof; a RNA sequence encoding a heterologous protein; and a poly-A tail,
wherein each of the R element of the 5′ LTR and the U5 element of the 5′ LTR is retroviral.

29. The lipid particle of claim 28, wherein the viral structural protein or a portion thereof is a retroviral structural protein or a portion thereof.

30. The lipid particle of any of claims 26, 28, and 29, wherein the RNA comprises a bicistronic element located between the RNA sequence encoding the viral structural protein or a portion thereof and the RNA sequence encoding the heterologous protein.

31. The lipid particle of claim 30, wherein the bicistronic element is an internal ribosome entry site (IRES) element or a sequence encoding a 2A self-cleaving peptide.

32. The lipid particle of claim 30 or claim 31, wherein the bicistronic element is a sequence encoding a 2A self-cleaving peptide, and the 2A self-cleaving peptide is T2A.

33. The lipid particle of claim 32, wherein T2A comprises the sequence set forth in SEQ ID NO: 76.

34. The lipid particle of any of claims 25 and 27-33, wherein the RNA encodes, from a 5′ to 3′ direction: the viral structural protein or portion thereof, T2A, and the heterologous protein.

35. The lipid particle of any of claims 26 and 28-34, wherein the viral structural protein is a retroviral gag.

36. The lipid particle of claim 35, wherein the RNA sequence encoding the viral structural protein or a portion thereof encodes an N-terminal portion of a retroviral gag.

37. The lipid particle of claim 35 or claim 36, wherein the RNA sequence encoding the viral structural protein or a portion thereof comprises the sequence set forth in SEQ ID NO:52.

38. The lipid particle of any of claims 1-37, wherein the RNA encodes the sequence set forth in SEQ ID NO:77 and the heterologous protein.

39. The lipid particle of any of claims 1-38, wherein the RNA is present as a first genomic viral RNA and the lipid particle further comprises a second genomic viral RNA.

40. The lipid particle of claim 39, wherein the first genomic viral RNA and the second viral genomic RNA genome are identical.

41. The lipid particle of claim 39, wherein the first genomic viral RNA and the second viral genomic RNA genome are different.

42. The lipid particle of any of claims 1-41, wherein the heterologous protein is a first heterologous protein and the lipid particle further comprises one or more additional heterologous protein(s), wherein the lipid particle comprises:
(a) a viral matrix (MA) protein, a MS2 coat protein (MS2_{cp}), and an RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}; and/or
(b) a fusion protein comprising a viral matrix (MA) protein and the one or more additional heterologous protein(s).

43. The lipid particle of claim 42, wherein the viral MA protein in (a) and/or (b) is derived from human immunodeficiency virus (HIV).

44. The lipid particle of claim 42 or claim 43, wherein the viral MA protein in (a) and/or (b) comprises the sequence set forth in SEQ ID NO:78.

45. The lipid particle of any of claims 42-44, wherein MS2_{cp} in (a) comprises the sequence set forth in SEQ ID NO:79.

46. The lipid particle of any of claims 42-45, wherein the fusion protein of (a) comprises the sequence set forth in SEQ ID NO:74.

47. The lipid particle of any one of claims 42-45, wherein the fusion protein of (a) comprises:
the amino acid sequence of SEQ ID NOs: 134 or 190; or
the amino acid sequence of SEQ ID NOs: 74 or 191; or
the amino acid sequence encoded by the nucleic acid sequence set forth in SEQ ID NO: 62 or 150.

48. The lipid particle of any of claims 1-47, wherein the RNA comprises a 5′ cap.

49. The lipid particle of any of claims 1-48, wherein the RNA is a self-inactivating lentiviral vector genome.

50. A lipid particle comprising a lipid bilayer enclosing a lumen; a fusion protein comprising a viral matrix (MA) protein and a MS2 coat protein (MS2_{cp}); and an RNA sequence encoding a heterologous protein.

51. The lipid particle of claim 50, wherein the RNA sequence encoding a heterologous protein comprises a MS2_{cp}-binding loop.

52. The lipid particle of claim 51, wherein the MS2_{cp}-binding loop comprises the RNA sequence transcribed from the nucleic acid set forth in SEQ ID NO: 185 or SEQ ID NO: 174.

53. The lipid particle of claim 51 or claim 52, wherein the MS2_{cp}-binding loop comprises the RNA sequence set forth in SEQ ID NO: 208.

54. The lipid particle of any one of claims 50-53, wherein the RNA sequence encoding a heterologous protein comprises a plurality of MS2_{cp}-binding loops.

55. The lipid particle of claim 54, wherein the plurality of MS2_{cp}-binding loops comprises at or a at least 2, 5, 6, 10, 12, 15, 20, or 24 MS2_{cp}-binding loops.

56. The lipid particle of any one of claims 50-55, wherein the RNA sequence encoding a heterologous protein comprises a plurality of MS2_{cp}-binding loops comprising between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops.

57. The lipid particle of any one of claims 54-56, wherein the plurality of MS2_{cp}-binding loops comprises the RNA sequence transcribed from a nucleic acid sequence selected from the group consisting of SEQ ID NOs: 175-178.

58. A lipid particle comprising a lipid bilayer enclosing a lumen; a viral matrix (MA) protein; an RNA-binding protein; and an RNA sequence encoding a heterologous protein, wherein the RNA-binding protein is incorporated into the lipid particle as a fusion protein with the viral MA protein, and wherein the RNA sequence encoding the heterologous protein comprises a binding site for binding to the RNA-binding protein.

59. The lipid particle of claim 58, wherein the RNA sequence encoding a heterologous protein comprises at or at least 2, 5, 6, 10, 12, 15, 20, or 24 binding sites for binding to the RNA-binding protein.

60. The lipid particle of claim 58 or claim 59, wherein the RNA sequence encoding a heterologous protein comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20, binding sites for binding to the RNA-binding protein.

61. The lipid particle of any one of claims 58-60, wherein the RNA-binding protein is MS2 coat protein (MS2_{cp}).

62. The lipid particle of claim 61, wherein MS2_{cp} comprises the sequence set forth in SEQ ID NO: 79.

63. The lipid particle of any one of claims 58-62, wherein the RNA-binding protein is MS2_{cp} and the binding site is an MS2_{cp}-binding loop for binding to the MS2_{cp}.

64. The lipid particle of claim 63, wherein the MS2_{cp}-binding loop comprises the RNA sequence transcribed from the nucleic acid set forth in SEQ ID NO: 185 or SEQ ID NO: 174.

65. The lipid particle of claim 63 or claim 64, wherein the MS2_{cp}-binding loop comprises the RNA sequence set forth in SEQ ID NO: 208.

66. The lipid particle of any one of claims 58-65, wherein the RNA sequence encoding a heterologous protein comprises a plurality of MS2_{cp}-binding loops.

67. The lipid particle of claim 66, wherein the plurality of MS2_{cp}-binding loops comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops.

68. The lipid particle of claim 66 or claim 67, wherein the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops.

69. The lipid particle of any one of claims 66-68, wherein the plurality of MS2_{cp}-binding loops comprises the RNA sequence transcribed from a nucleic acid sequence selected from the group consisting of SEQ ID NOs: 175-178.

70. The lipid particle of any one of claims 58-60, wherein the RNA-binding protein is lambda N protein (λN) or a functional variant thereof.

71. The lipid particle of claim 70, wherein the λN or a functional variant thereof comprises the amino acid sequence of SEQ ID NO: 187 or an amino acid sequence having at least 90% or 95% sequence identity to the amino acid sequence of SEQ ID NO: 187.

72. The lipid particle of claim 70 or claim 71, wherein the λN or a functional variant thereof comprises the amino acid sequence of SEQ ID NO: 187.

73. The lipid particle of claim 70, wherein the λN or a functional variant thereof comprises the amino acid sequence of SEQ ID NO: 188 or an amino acid sequence having at least 90% or 95% sequence identity to the amino acid sequence of SEQ ID NO: 188.

74. The lipid particle of claim 70 or claim 73, wherein the λN or a functional variant thereof comprises the amino acid sequence of SEQ ID NO: 188.

75. The lipid particle of any one of claims 58-60 and 70-74, wherein the RNA-binding protein is λN or a functional variant thereof and the binding site is a boxB binding site for binding to the λN or a functional variant thereof.

76. The lipid particle of claim 75, wherein the boxB binding site comprises the RNA sequence transcribed from the nucleic acid set forth in SEQ ID NO: 186.

77. The lipid particle of any one of claims 58-60 and 70-76, wherein the RNA sequence encoding a heterologous protein comprises a plurality of boxB binding sites.

78. The lipid particle of 77, wherein the plurality of boxB binding sites comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 boxB binding sites.

79. The lipid particle of claim 77 or claim 78, wherein the plurality of boxB binding sites comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 boxB binding sites.

80. The lipid particle of any one of claims 77-79, wherein the plurality of boxB binding sites comprises the RNA sequence transcribed from a nucleic acid sequence selected from the group consisting of SEQ ID NOs: 179-184.

81. A lipid particle comprising a lipid bilayer enclosing a lumen; a viral matrix (MA) protein; a MS2 coat protein (MS2_{cp}); and an RNA sequence encoding a heterologous protein, wherein the MS2_{cp} is incorporated into the lipid particle as a fusion protein with the viral MA protein, and wherein the RNA sequence encoding the heterologous protein comprises a MS2_{cp}-binding loop for binding to MS2_{cp}.

82. The lipid particle of any of claims 50-81, wherein the viral MA protein is attached to a portion of the lipid bilayer that is in contact with the lumen.

83. The lipid particle of any of claims 50-82, wherein the viral MA protein reversibly binds to the lipid bilayer.

84. The lipid particle of any of claims 50-83, wherein the fusion protein comprises, from a 5′ to 3′ direction: the viral MA protein and MS2_{cp}.

85. The lipid particle of any of claims 50-84, wherein the RNA sequence encoding a heterologous protein comprises a MS2_{cp}-binding loop, optionally 12 or 24 MS2_{cp}-binding loops.

86. The lipid particle of any of claims 50-85, wherein the viral MA protein is derived from human immunodeficiency virus (HIV).

87. The lipid particle of any of claims 50-86, wherein the viral MA protein comprises the sequence set forth in SEQ ID NO:78.

88. The lipid particle of any of claims 50-87, wherein MS2_{cp} comprises the sequence set forth in SEQ ID NO:79.

89. The lipid particle of any of claims 50-88, wherein the fusion protein comprises the sequence set forth in SEQ ID NO:74.

90. The lipid particle of any of claims 50-89, further comprising a transfer plasmid encoding a guide RNA (gRNA), optionally a single guide RNA (sgRNA), under the control of a U6 promoter.

91. The lipid particle of any of claims 50-90, wherein the heterologous protein is a first heterologous protein and the lipid particle further comprises one or more additional heterologous protein(s), wherein the lipid particle comprises:
(a) a RNA comprising a RNA sequence encoding the one or more additional heterologous protein(s) and a RNA sequence encoding a viral structural protein or a portion thereof; and/or
(b) a fusion protein comprising a viral matrix (MA) protein and the one or more additional heterologous protein(s).

92. The lipid particle of claim 91, wherein the viral structural protein is gag.

93. The lipid particle of claim 92, wherein the RNA sequence encoding the viral structural protein or a portion thereof in (a) encodes an N-terminal portion of gag.

94. The lipid particle of claim 92 or claim 93, wherein the RNA sequence encoding the viral structural protein or a portion thereof in (a) comprises the sequence set forth in SEQ ID NO:52.

95. The lipid particle of any of claims 92-94, wherein the viral MA protein in (b) is derived from human immunodeficiency virus (HIV).

96. The lipid particle of any of claims 92-95, wherein the viral MA protein in (b) comprises the sequence set forth in SEQ ID NO:78.

97. A lipid particle comprising a lipid bilayer enclosing a lumen a fusion protein comprising a viral matrix (MA) protein and a heterologous protein.

98. A lipid particle comprising a lipid bilayer enclosing a lumen; a viral matrix (MA) protein; and a heterologous protein, wherein the heterologous protein is incorporated into the lipid particle as a fusion protein with the viral MA protein.

99. The lipid particle of claim 97 or claim 98, wherein the viral MA protein is attached to a portion of the lipid bilayer that is in contact with the lumen.

100. The lipid particle of any of claims 97-99, wherein the viral MA protein reversibly binds to the lipid bilayer.

101. The lipid particle of any of claims 97-100, wherein the fusion protein comprises, from a 5′ to 3′ direction: the viral MA protein and the heterologous protein.

102. The lipid particle of any of claims 97-101, wherein the viral MA protein is derived from human immunodeficiency virus (HIV).

103. The lipid particle of any of claims 97-102, wherein the viral MA protein comprises the sequence set forth in SEQ ID NO:78.

104. The lipid particle of any of claims 97-103, further comprising a transfer plasmid encoding a guide RNA (gRNA), optionally a single guide RNA (sgRNA), under the control of a U6 promoter.

105. The lipid particle of any of claims 97-104, wherein the heterologous protein is a first heterologous protein and the lipid particle further comprises one or more additional heterologous protein(s), wherein the lipid particle comprises:
(a) a RNA comprising a RNA sequence encoding the one or more additional heterologous protein(s) and a RNA sequence encoding a viral structural protein or a portion thereof; and/or
(b) a viral matrix (MA) protein, a MS2 coat protein (MS2_{cp}), and a RNA sequence encoding the one or more additional heterologous protein(s), wherein the MS2_{cp} is incorporated into the lipid particle as a fusion protein with the viral MA protein, and wherein the RNA sequence encoding the one or more additional heterologous protein(s) comprises a MS2_{cp}-binding loop for binding to MS2_{cp}.

106. The lipid particle of claim 105, wherein the viral structural protein in (a) is gag.

107. The lipid particle of claim 106, wherein the RNA sequence encoding the viral structural protein or a portion thereof in (a) encodes an N-terminal portion of gag.

108. The lipid particle of claim 106 or claim 107, wherein the RNA sequence encoding the viral structural protein or a portion thereof in (a) comprises the sequence set forth in SEQ ID NO:52.

109. The lipid particle of any of claims 105-108, wherein the viral MA protein in (b) is derived from HIV.

110. The lipid particle of any of claims 105-109, wherein the viral MA protein in (b) comprises the sequence set forth in SEQ ID NO:78.

111. The lipid particle of any of claims 105-110, wherein MS2_{cp} in (b) comprises the sequence set forth in SEQ ID NO:79.

112. The lipid particle of any of claims 105-111, wherein the fusion protein of (b) comprises the sequence set forth in SEQ ID NO:74.

113. The lipid particle of any of claims 105-112, wherein the fusion protein of (b) comprises the sequence set forth in SEQ ID NO: 191.

114. A lipid particle comprising a lipid bilayer enclosing a lumen; a fusion protein comprising a viral envelope glycoprotein and an RNA-binding protein; and an RNA sequence encoding a heterologous protein, wherein the RNA sequence encoding the heterologous protein comprises a binding site for binding to the RNA-binding protein.

115. The lipid particle of claim 114, wherein the fusion protein comprises, from an N-terminus to C-terminus direction: the viral envelope glycoprotein and the RNA binding protein.

116. The lipid particle of claim 114 or claim 115, wherein the RNA-binding protein is fused to the C-terminus of the viral envelope glycoprotein.

117. A lipid particle comprising a lipid bilayer enclosing a lumen; a fusion protein comprising a VSV-G protein or a functional variant thereof and an RNA-binding protein; and an RNA sequence encoding a heterologous protein, wherein the RNA sequence encoding the heterologous protein comprises a binding site for binding to the RNA-binding protein.

118. The lipid particle of claim 117, wherein the VSV-G or a functional variant thereof comprises the amino acid sequence of SEQ ID NO: 199, or an amino acid sequence having at least 90%, 91%, 92%, 93%, 94%, 95%, 96%, 97%, 98%, or 99% sequence identity to the amino acid sequence of SEQ ID NO: 199.

119. The lipid particle of claim 117 or claim 118, wherein the VSV-G or a functional variant thereof comprises the amino acid sequence of SEQ ID NO: 199.

120. The lipid particle of any one of claims 114-119, wherein the RNA sequence encoding a heterologous protein comprises at or at least 2, 5, 6, 10, 12, 15, 20, or 24 binding sites for binding to the RNA-binding protein.

121. The lipid particle of any one of claims 114-120, wherein the RNA sequence encoding a heterologous protein comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20, binding sites for binding to the RNA-binding protein.

122. The lipid particle of any one of claims 114-116, wherein the lipid particle is pseudotyped with the viral envelope glycoprotein.

123. The lipid particle of any one of claims 117-121, wherein the fusion protein comprises, from an N-terminus to C-terminus direction: the VSV-G protein or a functional variant thereof and the RNA binding protein.

124. The lipid particle of any one of claims 117-121, wherein the RNA-binding protein is fused to the C-terminus of the VSV-G protein or a functional variant thereof.

125. The lipid particle of any one of claims 114-124, wherein the RNA-binding protein is MS2 coat protein (MS2_{cp}).

126. The lipid particle of claim 125, wherein MS2_{cp} comprises the sequence set forth in SEQ ID NO: 79.

127. The lipid particle of claim 125 or claim 126, wherein the MS2_{cp} is a homodimer.

128. The lipid particle of claim 125 or claim 126, wherein the MS2_{cp} is a tandem dimer.

129. The lipid particle of any one of claims 114-128, wherein the binding site is an MS2_{cp}-binding loop for binding to the MS2_{cp}.

130. The lipid particle of any one of claims 114-129, wherein the RNA sequence encoding a heterologous protein comprises a plurality of MS2_{cp}-binding loops for binding to the MS2_{cp}.

131. The lipid particle of claim 130, wherein the plurality of MS2_{cp}-binding loops comprises at or at least 2, 4, 6, 8, 10, 12, 14, 16, 18, 20, 22, or 24 MS2_{cp}-binding loops.

132. The lipid particle of claim 130 or claim 131, wherein the plurality of MS2_{cp}-binding loops comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 MS2_{cp}-binding loops.

133. The lipid particle of any one of claims 130-132, wherein the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops.

134. The lipid particle of any one of claims 114-124, wherein the RNA-binding protein is lambda N protein (λN) or a functional variant thereof.

135. The lipid particle of claim 134, wherein the λN or a functional variant thereof comprises the amino acid sequence of SEQ ID NO: 187 or an amino acid sequence having at least 90% or 95% sequence identity to the amino acid sequence of SEQ ID NO: 187.

136. The lipid particle of claim 134 or claim 135, wherein the λN or a functional variant thereof comprises the amino acid sequence of SEQ ID NO: 187.

137. The lipid particle of claim 134, wherein the λN or a functional variant thereof comprises the amino acid sequence of SEQ ID NO: 188 or an amino acid sequence having at least 90% or 95% sequence identity to the amino acid sequence of SEQ ID NO: 188.

138. The lipid particle of claim 134, wherein the λN or a functional variant thereof comprises the amino acid sequence of SEQ ID NO: 188.

139. The lipid particle of any one of claims 114-125 and 134-138, wherein the RNA-binding protein is λN or a functional variant thereof and the binding site is a boxB binding site for binding to the λN or a functional variant thereof.

140. The lipid particle of any one of claims 114-124 and 134-139, wherein the RNA sequence encoding a heterologous protein comprises a plurality of boxB binding sites.

141. The lipid particle of any one of claim 140, wherein the plurality of boxB binding sites comprises between or between about 1 and 50, 1 and 40, 1 and 30, 1 and 25, 1 and 20, 2 and 50, 2 and 40, 2 and 30, 2 and 25, 2 and 20, 4 and 50, 4 and 40, 4 and 30, 4 and 25, 4 and 20, 5 and 50, 5 and 40, 5 and 30, 5 and 25, 5 and 20, 6 and 50, 6 and 40, 6 and 30, 6 and 25, 6 and 20, 10 and 50, 10 and 40, 10 and 30, 10 and 25, 10 and 20, 12 and 50, 12 and 40, 12 and 30, 12 and 25, 12 and 20, 15 and 50, 15 and 40, 15 and 30, 15 and 25, or 15 and 20 boxB binding sites.

142. The lipid particle of claim 140 or claim 141, wherein the plurality of MS2_{cp}-binding loops comprises at or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 MS2_{cp}-binding loops.

143. The lipid particle of any one of claims 140-142, wherein the plurality of boxB binding sites comprises the RNA sequence transcribed from a nucleic acid sequence selected from the group consisting of SEQ ID NOs: 179-184.

144. The lipid particle of any of claims 1-143, wherein the heterologous protein is a genome-modifying protein.

145. The lipid particle of claim 144, wherein the genome-modifying protein comprises a recombinant nuclease, a nickase, an integrase, reverse transcriptase, or a combination thereof.

146. The lipid particle of claim 144 or claim 145, wherein the genome-modifying protein comprises a zinc-finger nuclease (ZFN), a transcription-activator like effector nucleases (TALEN), or a CRISPR-associated (Cas) protein.

147. The lipid particle of any of claims 144-146, wherein the genome-modifying protein is a Cas protein.

148. The lipid particle of any of claims 144-147, wherein the genome-modifying protein is (i) Cas9, optionally saCas9 or spCas9; or (ii) cpf1.

149. The lipid particle of any of claims 1-148, further comprising a guide RNA (gRNA) in the lumen.

150. The lipid particle of claim 149, wherein the gRNA is a single guide RNA (sgRNA).

151. The lipid particle of any of claims 1-150, wherein the lipid particle is pseudotyped with a viral envelope glycoprotein.

152. The lipid particle of claim 151, wherein the viral envelope glycoprotein is a VSV-G protein or a functional variant thereof.

153. The lipid particle of claim 152, wherein the VSV-G or a functional variant thereof comprises the amino acid sequence of SEQ ID NO: 199, or an amino acid sequence having at least 90%, 91%, 92%, 93%, 94%, 95%, 96%, 97%, 98%, or 99% sequence identity to the amino acid sequence of SEQ ID NO: 199.

154. The lipid particle of claim 152 or claim 153, wherein the VSV-G or a functional variant thereof comprises the amino acid sequence of SEQ ID NO: 199.

155. The lipid particle of claim 151, wherein the viral envelope glycoprotein is a Cocal virus G protein or a functional variant thereof.

156. The lipid particle of claim 151, wherein the viral envelope glycoprotein is an Alphavirus fusion protein (e.g. Sindbis virus) or a functional variant thereof.

157. The lipid particle of claim 151, wherein the viral envelope glycoprotein is a Paramyxoviridae fusion protein (e.g., a Morbillivirus or a Henipavirus) or a functional variant thereof.

158. The lipid particle of claim 151 or claim 157, wherein the viral envelope glycoprotein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof.

159. The lipid particle of claim 151 or claim 157, wherein the viral envelope glycoprotein is a Henipavirus fusion protein (e.g., Nipah virus, Hendra virus, Cedar virus, Kumasi virus, Mòjiāng virus, Langya virus) or a functional variant thereof.

160. The lipid particle of any of claims 151-159, wherein the viral envelope glycoprotein comprises one or more modifications to reduce binding to its native receptor.

161. The lipid particle of any of claims 151, 157, 159, and 160, wherein the viral envelope glycoprotein comprises a Nipah virus F glycoprotein (NiV-F) or a biologically active portion thereof and a Nipah virus G glycoprotein (NiV-G) or a biologically active portion thereof.

162. The lipid particle of claim 161, wherein the Niv-F or a biologically active portion thereof comprises the amino acid sequence set forth in SEQ ID NO: 145, or an amino acid sequence having at least 90%, 91%, 92%, 93%, 94%, 95%, 96%, 97%, 98%, or 99% sequence identity to the amino acid sequence set forth in SEQ ID NO: 145; and/or the Niv-G or a biologically active portion thereof comprises the amino acid sequence set forth in SEQ ID NO: 147, or an amino acid sequence having at least 90%, 91%, 92%, 93%, 94%, 95%, 96%, 97%, 98%, or 99% sequence identity to the amino acid sequence set forth in SEQ ID NO: 147.

163. The lipid particle of claim 161, wherein the Niv-F or a biologically active portion thereof comprises the amino acid sequence set forth in SEQ ID NO: 145; and/or the Niv-G or a biologically active portion thereof comprises the amino acid sequence set forth in SEQ ID NO: 147.

164. The lipid particle of claim 161, wherein the NiV-G or the biologically active portion thereof is a wild-type NiV-G protein or a functionally active variant or biologically active portion thereof.

165. The lipid particle of claim 161 or claim 164, wherein the NiV-G protein or the biologically active portion is truncated and lacks up to 40 contiguous amino acid residues at or near the N-terminus of the wild-type NiV-G protein.

166. The lipid particle of any of claims 161-165, wherein the NiV-G protein or the biologically active portion has a 5 amino acid truncation at or near the N-terminus of the wild-type NiV-G protein, optionally wherein the NiV-G protein or the biologically active portion thereof has the amino acid sequence set forth in SEQ ID NO: 12, or a sequence of amino acids that exhibits at least at or about 80%, 85%, 90% or 95% sequence identity to the sequence set forth in SEQ ID NO: 12.

167. The lipid particle of any of claims 161-166, wherein the NiV-G protein or the biologically active portion has a 10 amino acid truncation at or near the N-terminus of the wild-type NiV-G protein, optionally wherein the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO: 44.

168. The lipid particle of any of claims 161-167, wherein the NiV-G protein or the biologically active portion has a 15 amino acid truncation at or near the N-terminus of the wild-type NiV-G protein, optionally wherein the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO: 45.

169. The lipid particle of any of claims 161-168, wherein the NiV-G protein or the biologically active portion has a 20 amino acid truncation at or near the N-terminus of the wild-type NiV-G protein, optionally wherein the NiV-G protein or the biologically active portion thereof has the amino acid sequence set forth in SEQ ID NO:13, or a sequence of amino acids that exhibits at least at or about 80%, 85%, 90% or 95% sequence identity to the sequence set forth in SEQ ID NO: 13.

170. The lipid particle of any of claims 161-168, wherein the NiV-G protein or the biologically active portion has a 25 amino acid truncation at or near the N-terminus of the wild-type NiV-G protein, optionally wherein the NiV-G protein or the biologically active portion thereof has the amino acid sequence set forth in SEQ ID NO: 14, or a sequence of amino acids that exhibits at least at or about 80%, 85%, 90% or 95% sequence identity to the sequence set forth in SEQ ID NO: 14.

171. The lipid particle of any of claims 161-168, wherein the NiV-G protein or the biologically active portion has a 30 amino acid truncation at or near the N-terminus of the wild-type NiV-G protein, optionally wherein the NiV-G protein or the biologically active portion thereof has the amino acid sequence set forth in SEQ ID NO:43, or a sequence of amino acids that exhibits at least at or about 80%, 85%, 90% or 95% sequence identity to the sequence set forth in SEQ ID NO: 43.

172. The lipid particle of any of claims 161-168, wherein the NiV-G protein or the biologically active portion has a 34 amino acid truncation at or near the N-terminus of the wild-type NiV-G protein, optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO: 42.

173. The lipid particle of any of claims 161-172, wherein the NiV-G-protein or the biologically active portion thereof is a mutant NiV-G protein that exhibits reduced binding to Ephrin B2 or Ephrin B3.

174. The lipid particle of claim 173, wherein the mutant NiV-G protein or the biologically active portion comprises one or more amino acid substitutions corresponding to amino acid substitutions selected from the group consisting of E501A, W504A, Q530A and E533A with reference to numbering set forth in SEQ ID NO:4.

175. The lipid particle of claim 173 or claim 174, wherein the mutant NiV-G protein or the biologically active portion comprises the amino acid sequence set forth in SEQ ID NO: 17 or a sequence of amino acids that exhibits at least at or about 80%, 85%, 90% or 95% sequence identity to the sequence set forth in SEQ ID NO: 17.

176. The lipid particle of claim 173 or claim 174, wherein the NiV-G protein or the biologically active portion has the amino acid sequence set forth in SEQ ID NO: 18 or a sequence of amino acids that exhibits at least at or about 80%, 85%, 90% or 95% sequence identity to the sequence set forth in SEQ ID NO: 18.

177. The lipid particle of any of claims 161-176, wherein the NiV-F protein or the biologically active portion thereof is a wild-type NiV-F protein or is a functionally active variant or a biologically active portion thereof.

178. The lipid particle of any of claims 161-177, wherein the NiV-F protein or the biologically active portion thereof has a 20 amino acid truncation at or near the C-terminus of the wild-type NiV-F protein, optionally wherein the NiV-F protein or the biologically active portion thereof comprises the sequence set forth in SEQ ID NO: 20 or a sequence of amino acids that exhibits at least at or about 80%, 85%, 90% or 95% sequence identity to the sequence set forth in SEQ ID NO: 20.

179. The lipid particle of any of claims 161-178, wherein the NiV-F protein or the biologically active portion thereof comprises:
i) a 20 amino acid truncation at or near the C-terminus of the wild-type NiV-F protein; and
ii) a point mutation on an N-linked glycosylation site,
optionally wherein the NiV-F protein or the biologically active portion thereof comprises the sequence set forth in SEQ ID NO: 15, or a sequence of amino acids that exhibits at least at or about 80%, 85%, 90% or 95% sequence identity to the sequence set forth in SEQ ID NO: 15.

180. The lipid particle of any of claims 161-179, wherein the NiV-F protein or the biologically active portion thereof has a 22 amino acid truncation at or near the C-terminus of the wild-type NiV-F protein, optionally wherein the NiV-F protein or the biologically active portion thereof comprises the sequence set forth in SEQ ID NO: 16, 19, or 21 or a sequence of amino acids that exhibits at least at or about 80%, 85%, 90% or 95% sequence identity to the sequence set forth in SEQ ID NO: 16, 19, or 21.

181. The lipid particle of any of claims 161-177 and 180, wherein the NiV-F protein or the biologically active portion thereof comprises the amino acid sequence set forth in SEQ ID NO:21, or a sequence of amino acids that exhibits at least at or about 80%, 85%, 90% or 95% sequence identity to the sequence set forth in SEQ ID NO:21.

182. The lipid particle of any of claims 161-177, 180, and 181, wherein the Niv-G protein comprises the amino acid sequence set forth in SEQ ID NO: 17, and the Niv-F protein comprises the amino acid sequence set forth in SEQ ID NO:21.

183. The lipid particle of any of claims 1-182, further comprising a targeting moiety.

184. The lipid particle of claim 183, wherein the targeting moiety is selected from the group consisting of a CD3-binding agent, a CD8-binding agent, and a CD4-binding agent.

185. The lipid particle of claim 183 or claim 184, wherein the targeting moiety is a CD3-binding agent, optionally an anti-CD3 antibody or an antigen-binding fragment.

186. The lipid particle of claim 183 or claim 184, wherein the targeting moiety is a CD8-binding agent, optionally an anti-CD8 antibody or an antigen-binding fragment.

187. The lipid particle of claim 183 or claim 184, wherein the targeting moiety is a CD4-binding agent, optionally an anti-CD4 antibody or an antigen-binding fragment.

188. The lipid particle of any of claims 183-187, wherein the targeting moiety is exposed on the surface of the lipid particle.

189. The lipid particle of any of claims 183-188, wherein the targeting moiety is fused to a transmembrane domain incorporated into the bilayer of the lipid particle.

190. The lipid particle of any of claims 1-189, wherein the lipid particle is a retroviral vector or a retroviral-like particle.

191. The lipid particle of any of claims 1-190, wherein the retroviral vector or the retroviral-like particle is replication-deficient.

192. The lipid particle of any of claims 1-191, where the lipid particle does not comprise reverse transcriptase or does not comprise reverse transcriptase activity.

193. The lipid particle of any of claims 1-191, where the lipid particle does not comprise a protein with reverse transcriptase activity.

194. The lipid particle of claim 192 or claim 193, wherein the lipid particle does not comprise reverse transcriptase.

195. The lipid particle of claim 192 or claim 193, wherein the lipid particle comprises non-functional reverse transcriptase, optionally wherein the reverse transcriptase is mutated.

196. The lipid particle of any of claims 190-195, wherein the retroviral vector or retroviral-like particle comprises a RNA that is a self-inactivating lentiviral vector genome.

197. The lipid particle of any of claims 191-196, wherein the retroviral vector or retroviral-like particle comprises a RNA comprising a 3′LTR, and the 3′ LTR does not comprise a functional U3 domain, optionally wherein the U3 domain comprises a deletion.

198. The lipid particle of any of claims 1-197, wherein the lipid particle is a retroviral particle, and the retroviral particle is a lentiviral particle.

199. The lipid particle of any of claims 1-197, wherein the lipid particle is a retrovirus-like particle (VLP).

200. The lipid particle of any of claims 1-199, wherein the lipid bilayer is derived from a host cell.

201. The lipid particle of claim 200, wherein the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

202. A method of producing a lipid particle comprising a lipid bilayer enclosing a lumen and a viral ribonucleic acid (RNA), comprising:
(1) providing a host cell comprising (a) a nucleic acid sequence selected from the group consisting of: a 5′ long terminal repeat (5′ LTR); a psi packaging signal sequence; a gag start codon; a RNA sequence encoding a heterologous protein; a 3′ long terminal repeat (3′ LTR); or a combination thereof; and (b) a nucleic acid sequence encoding a protein selected from the group consisting of gag, pol, rev, tat, a viral envelope glycoprotein, or a combination thereof; and
(2) culturing the host cell under conditions to induce packaging of the lipid particle.

203. The method of claim 202, further comprising a RNA sequence encoding a viral structural protein or a portion thereof, which is located between the gag start codon and the RNA sequence encoding a heterologous protein.

204. The method of claim 202, wherein the gag start codon and the RNA sequence encoding a heterologous protein are part of the same RNA, and the RNA does not comprise nucleotides between the gag start codon and the RNA sequence encoding a heterologous protein.

205. A method of producing a lipid particle comprising a lipid bilayer enclosing a lumen and viral ribonucleic acid (RNA), comprising:
(1) providing a host cell comprising (a) a RNA sequence encoding a heterologous protein and a viral structural protein or a portion thereof; and (b) a nucleic acid sequence encoding a protein selected from the group consisting of gag, pol, rev, tat, a viral envelope glycoprotein, or a combination thereof; and
(2) culturing the host cell under conditions to induce packaging of the lipid particle.

206. The method of claim 205, wherein the RNA sequence encoding the viral structural protein or portion thereof is located 5′ to the RNA sequence encoding the heterologous protein.

207. The method of any of claims 203, 205, and 206, wherein a bicistronic element is located between the RNA sequence encoding the viral structural protein or portion thereof and the RNA sequence encoding the heterologous protein.

208. The method of claim 207, wherein the bicistronic element is an internal ribosome entry site (IRES) element or a sequence encoding a 2A self-cleaving peptide.

209. The method of claim 207 or claim 208, wherein the bicistronic element is a sequence encoding a 2A self-cleaving peptide, and the 2A self-cleaving peptide is T2A.

210. The method of claim 209, wherein T2A comprises the sequence set forth in SEQ ID NO: 76.

211. The method of any of claims 203 and 205-210, wherein the RNA sequence encodes, from a 5′ to 3′ direction: the viral structural protein or portion thereof, T2A, and the heterologous protein.

212. The method of any of claims 203 and 205-211, wherein the viral structural protein is gag.

213. The method of claim 212, wherein the RNA sequence encoding the viral structural protein or a portion thereof encodes an N-terminal portion of gag.

214. The method of claim 212 or claim 213, wherein the RNA sequence encoding the viral structural protein or a portion thereof comprises the sequence set forth in SEQ ID NO:52.

215. The method of any of claims 202-214, wherein the host cell comprises a nucleic acid sequence that comprises the sequence set forth in SEQ ID NO:77 and encodes the heterologous protein.

216. A method of producing a lipid particle comprising a lipid bilayer enclosing a lumen and a viral transfer plasmid, comprising
(1) providing a host cell comprising (a) a nucleic acid sequence encoding a fusion protein comprising a viral matrix (MA) protein and an RNA binding protein; (b) a nucleic acid sequence encoding a heterologous protein; and (c) a nucleic acid sequence encoding a protein selected from the group consisting of gag, pol, Rev, Tat, a viral envelope glycoprotein, or a combination thereof; and
(2) culturing the host cell under conditions to induce packaging of the lipid particle.

217. The method of claim 216, wherein the RNA binding protein is a MS2 coat protein (MS2_{cp}).

218. The method of claim 217, wherein MS2_{cp} comprises the sequence set forth in SEQ ID NO: 79.

219. The method of any one of claims 216-218, wherein the nucleic acid sequence encoding a heterologous protein comprises a MS2_{cp}-binding loop, optionally at or at least 2, 3, 4, 5, 6, 7, 8, 9, 10, 11, 12, 13, 14, 15, 16, 17, 18, 19, 20, 21, 22, 23, or 24 MS2_{cp}-binding loops.

220. The method of claim 219, wherein the MS2_{cp}-binding loop comprises the RNA sequence transcribed from the nucleic acid set forth in SEQ ID NO: 185 or SEQ ID NO: 174.

221. The method of claim 219 or claim 220, wherein the MS2_{cp}-binding loop comprises the RNA sequence set forth in SEQ ID NO: 208.

222. The method of any one of claims 216-221, wherein the nucleic acid sequence encoding a heterologous protein comprises a plurality of MS2_{cp}-binding loops, and wherein the plurality of MS2_{cp}-binding loops comprises the RNA sequence transcribed from a nucleic acid sequence selected from the group consisting of SEQ ID NOs: 175-178.

223. The method of claim 216, wherein the RNA binding protein is a lambda N protein (λN) or a functional variant thereof.

224. The method of claim 223, wherein the λN or a functional variant thereof comprises the amino acid sequence of SEQ ID NO: 187 or an amino acid sequence having at least 90% or 95% sequence identity to the amino acid sequence of SEQ ID NO: 187.

225. The method of claim 223 or claim 224, wherein the λN or a functional variant thereof comprises the amino acid sequence of SEQ ID NO: 187.

226. The method of claim 223, wherein the λN or a functional variant thereof comprises the amino acid sequence of SEQ ID NO: 188 or an amino acid sequence having at least 90% or 95% sequence identity to the amino acid sequence of SEQ ID NO: 188.

227. The method of claim 223 or claim 226, wherein the λN or a functional variant thereof comprises the amino acid sequence of SEQ ID NO: 188.

228. The method of any one of claims 216 and 223-227, wherein the nucleic acid sequence encoding a heterologous protein comprises a boxB binding site for binding to λN or a functional variant thereof, optionally at or at least 2, 3, 4, 5, 6, 7, 8, 9, 10, 11, 12, 13, 14, 15, 16, 17, 18, 19, 20, 21, 22, 23, or 24 boxB binding sites.

229. The method of claim 228, wherein the boxB binding site comprises the RNA sequence transcribed from the nucleic acid set forth in SEQ ID NO: 186.

230. The lipid particle of any one of claims 216 and 223-229, wherein the nucleic acid sequence encoding a heterologous protein comprises a plurality of boxB binding sites for binding to λN or a functional variant thereof, and the plurality of boxB binding sites comprises the RNA sequence transcribed from a nucleic acid sequence selected from the group consisting of SEQ ID NOs: 179-184.

231. A method of producing a lipid particle comprising a lipid bilayer enclosing a lumen and a viral transfer plasmid, comprising:
(1) providing a host cell comprising (a) a nucleic acid sequence encoding a fusion protein comprising a viral matrix (MA) protein and a MS2 coat protein (MS2_{cp}); (b) a nucleic acid sequence encoding a heterologous protein; and (c) a nucleic acid sequence encoding a protein selected from the group consisting of gag, pol, Rev, Tat, a viral envelope glycoprotein, or a combination thereof; and
(2) culturing the host cell under conditions to induce packaging of the lipid particle.

232. The method of claim 231, wherein the fusion protein comprises, from a 5′ to 3′ direction: the viral MA protein and MS2_{cp}.

233. The method of claim 231 or claim 232, wherein the nucleic acid sequence encoding a heterologous protein comprises a MS2_{cp}-binding loop, optionally 12 or 24 MS2_{cp}-binding loops.

234. The method of any of claims 231-233, wherein the viral MA protein is derived from human immunodeficiency virus (HIV).

235. The method of any of claims 231-234, wherein the viral MA protein comprises the sequence set forth in SEQ ID NO:78.

236. The method of any of claims 231-235, wherein MS2_{cp} comprises the sequence set forth in SEQ ID NO:79.

237. The method of any of claims 231-236, wherein the fusion protein comprises the sequence set forth in SEQ ID NO:74.

238. A method of producing a lipid particle comprising a lipid bilayer enclosing a lumen and a viral transfer plasmid, comprising:
(1) providing a host cell comprising (a) a nucleic acid sequence encoding a fusion protein comprising a viral envelope glycoprotein and an RNA binding protein; (b) a nucleic acid sequence encoding a heterologous protein; and
(2) culturing the host cell under conditions to induce packaging of the lipid particle.

239. The method of claim 238, wherein the host cells further comprises a nucleic acid sequence encoding a protein selected from the group consisting of gag, pol, Rev, Tat, or a combination thereof.

240. The method of claim 238 or claim 239, wherein the fusion protein comprises, from a 5′ to 3′ direction: the viral envelope glycoprotein and the RNA binding protein.

241. The method of any one of claims 238-240, wherein the viral envelope glycoprotein is a VSV-G protein or a functional variant thereof.

242. The method of any one of claims 238-241, wherein the RNA binding protein is a MS2 coat protein (MS2_{cp}).

243. The method of claim 242, wherein MS2_{cp} comprises the sequence set forth in SEQ ID NO: 79.

244. The method of any one of claims 238-243, wherein the nucleic acid sequence encoding a heterologous protein comprises a MS2_{cp}-binding loop, optionally at or at least 12 or 24 MS2_{cp}-binding loops.

245. The method of claim 244, wherein the MS2_{cp}-binding loop comprises the RNA sequence transcribed from the nucleic acid set forth in SEQ ID NO: 185 or SEQ ID NO: 174.

246. The lipid particle of claim 244 or claim 245, wherein the MS2_{cp}-binding loop comprises the RNA sequence set forth in SEQ ID NO: 208.

247. The method of any one of claims 238-246, wherein the nucleic acid sequence encoding a heterologous protein comprises a plurality of MS2_{cp}-binding loops, and wherein the plurality of MS2_{cp}-binding loops comprises the RNA sequence transcribed from a nucleic acid sequence selected from the group consisting of SEQ ID NOs: 175-178.

248. The method of any of claims 238-247, wherein the viral envelope glycoprotein is derived from human immunodeficiency virus (HIV).

249. The method of any one of claims 238-241, wherein the RNA binding protein is a lambda N protein (λN) or a functional variant thereof.

250. The method of any one of claims 238-241 and 249, wherein the nucleic acid sequence encoding a heterologous protein comprises a boxB binding site for binding to λN or a functional variant thereof, optionally at or at least 2, 3, 4, 5, 6, 7, 8, 9, 10, 11, 12, 13, 14, 15, 16, 17, 18, 19, 20, 21, 22, 23, or 24 boxB binding sites.

251. The method of claim 250, wherein the boxB binding site comprises the RNA sequence transcribed from the nucleic acid set forth in SEQ ID NO: 186.

252. The lipid particle of any one of claims 238-241, 250, and 251, wherein the nucleic acid sequence encoding a heterologous protein comprises a plurality of boxB binding sites for binding to λN or a functional variant thereof, and the plurality of boxB binding sites comprises the RNA sequence transcribed from a nucleic acid sequence selected from the group consisting of SEQ ID NOs: 179-184.

253. A method of producing a lipid particle comprising a lipid bilayer enclosing a lumen and a viral transfer plasmid, comprising:
(1) providing a host cell comprising (a) a nucleic acid sequence encoding a fusion protein comprising viral matrix (MA) protein and a heterologous protein; and (b) a nucleic acid sequence encoding a protein selected from the group consisting of gag, pol, Rev, Tat, a viral envelope glycoprotein, or a combination thereof; and
(2) culturing the host cell under conditions to induce packaging of the lipid particle.

254. The method of claim 253, wherein the fusion protein comprises, from a 5′ to 3′ direction: the viral MA protein and the heterologous protein.

255. The method of claim 253 or claim 254, wherein the viral MA protein is derived from human immunodeficiency virus (HIV).

256. The method of any of claims 253-255, wherein the viral MA protein comprises the sequence set forth in SEQ ID NO:78.

257. The method of any of claims 202-256, wherein the viral envelope glycoprotein is VSV-G.

258. The method of any of claims 202-257, wherein the host cell is selected from the group consisting of a CHO cell, a BHK cell, a MDCK cell, a C3H 10T1/2 cell, a FLY cell, a Psi-2 cell, a BOSC 23 cell, a PA317 cell, a WEHI cell, a COS cell, a BSC 1 cell, a BSC 40 cell, a BMT 10 cell, a VERO cell, a W138 cell, a MRC5 cell, a A549 cell, a HT1080 cell, a 293 cell, a 293T cell, a B-50 cell, a 3T3 cell, a NIH3T3 cell, a HepG2 cell, a Saos-2 cell, a Huh7 cell, a HeLa cell, a W163 cell, a 211 cell, and a 211A cell.

259. The method of any of claims 202-258, wherein the nucleic acid sequence in (b) comprises a 5′ promoter.

260. The method of any of claims 231-259, wherein the nucleic acid sequence in (c) comprises a 5′ promoter.

261. The method of claim 259 or claim 260, wherein the promoter is a cytomegalovirus (CMV) promoter.

262. A lipid particle produced by the methods of any of claims 202-261.

263. A composition comprising the lipid particle of any of claims 1-201 and 262.

264. A method of introducing a heterologous protein into a target cell, the method comprising contacting the target cell with the lipid particle of any of claims 1-201 and 262 or the composition of claim 263.

265. A method of genetically engineering a target cell, the method comprising contacting the target cell with the lipid particle of any of claims 1-201 and 262 or the composition of claim 263.

266. The method of claim 264 or claim 265, wherein the contacting is in vitro or ex vivo.

267. The method of claim 264 or claim 265, wherein the contacting is in vivo.

268. A deoxyribonucleic acid (DNA) sequence encoding a gag start codon and a heterologous protein.

269. The DNA sequence of claim 268, further encoding a viral structural protein or a portion thereof, wherein the portion of the DNA sequence encoding the viral structural protein is located between the portions of the DNA sequence encoding the gag start codon and the heterologous protein.

270. The DNA sequence of claim 268 or 269, further encoding a bicistronic element, wherein the portion of the DNA sequence encoding the bicistronic element is located between the portions of the DNA sequence encoding the viral structural protein or a portion thereof and the heterologous protein.

271. The DNA sequence of claim 270, wherein the bicistronic element is an internal ribosome entry site (IRES) element or a sequence encoding a 2A self-cleaving peptide.

272. The DNA sequence of claim 271, wherein the 2A self-cleaving peptide is T2A.

273. The DNA sequence of claim 272, wherein T2A comprises the sequence set forth in SEQ ID NO: 76.

274. The DNA sequence of any of claims 269-273, wherein the DNA sequence encodes from a 5′ to 3′ direction: the viral structural protein or portion thereof, T2A, and the heterologous protein.

275. The DNA sequence of any of claims 269-274, wherein the viral structural protein is gag.

276. The DNA sequence of claim 275, encoding an N-terminal portion of gag.

277. The DNA sequence of claim 275 or claim 276, wherein the N-terminal portion of gag comprises the sequence set forth in SEQ ID NO:52.

278. The DNA sequence of any of claims 268-277, which encodes the sequence set forth in SEQ ID NO:77 and the heterologous protein.

279. The DNA sequence of claim 278, which does not comprise nucleotides between the encoded gag start codon and the encoded heterologous protein.

280. The DNA sequence of any of claims 268-279, comprising a promoter.

281. The DNA sequence of claim 280, wherein the promoter is a cytomegalovirus (CMV) promoter.

282. A DNA sequence encoding a viral matrix (MA) protein, an RNA binding protein, and a cleavage site between the portions of the DNA sequence encoding the MA protein and the RNA binding protein.

283. The DNA sequence of claim 282, which encodes a fusion protein comprising, from 5′ to 3′, the viral MA protein and RNA binding protein.

284. The DNA sequence of claim 282 or claim 283, wherein the RNA binding protein is a MS2 coat protein (MS2_{cp}).

285. The DNA sequence of claim 284, wherein MS2_{cp} comprises the sequence set forth in SEQ ID NO:79.

286. The DNA sequence of claim 282 or claim 283, wherein the RNA binding protein is a lambda N protein (λN) or a functional variant thereof.

287. The DNA sequence of claim 286, wherein the λN or a functional variant thereof comprises the amino acid sequence of SEQ ID NO: 187 or 188.

288. The DNA sequence of any of claims 282-287 wherein the encoded viral MA protein comprises the sequence set forth in SEQ ID NO:78.

289. The DNA sequence of any one of claims 282-288, comprising the nucleic acid sequence set forth in any one of SEQ ID NOs: 62, 150, 153, and 154.

290. A DNA sequence encoding a viral matrix (MA) protein, a MS2 coat protein (MS2_{cp}), and a cleavage site between the portions of the DNA sequence encoding the MA protein and the MS2_{cp}.

291. The DNA sequence of claim 290, which encodes a fusion protein comprising, from 5′ to 3′, the viral MA protein and MS2_{cp}.

292. The DNA sequence polynucleotide of claim 290 or claim 291, wherein the encoded MS2_{cp} comprises a MS2_{cp}-binding loop, optionally 12 or 24 MS2_{cp}-binding loops.

293. The DNA sequence of any of claims 290-292, wherein the encoded viral MA protein is derived from human immunodeficiency virus (HIV).

294. The DNA sequence of any of claims 290-293, wherein the encoded viral MA protein comprises the sequence set forth in SEQ ID NO:78.

295. The DNA sequence of any of claims 290-294, wherein the encoded MS2_{cp} comprises the sequence set forth in SEQ ID NO:79.

296. The DNA sequence of any of claims 290-295, wherein the encoded fusion protein comprises the sequence set forth in SEQ ID NO:74.

297. A DNA sequence encoding a viral envelope glycoprotein, an RNA binding protein, and a cleavage site between the portions of the DNA sequence encoding the viral envelope glycoprotein and the RNA binding protein.

298. The DNA sequence of claim 297, wherein the fusion protein comprises, from a 5′ to 3′ direction: the viral envelope glycoprotein and the RNA binding protein.

299. The DNA sequence of claim 297 or claim 298, wherein the viral envelope glycoprotein is a VSV-G protein or a functional variant thereof.

300. The DNA sequence of any of claims 297-299, wherein the viral envelope glycoprotein is derived from human immunodeficiency virus (HIV).

301. The DNA sequence of any one of claims 297-300, wherein the RNA binding protein is a MS2 coat protein (MS2_{cp}).

302. The DNA sequence of claim 301, wherein MS2_{cp} comprises the sequence set forth in SEQ ID NO:79.

303. The DNA sequence of any one of claims 297-300, wherein the RNA binding protein is a lambda N protein (λN) or a functional variant thereof.

304. The DNA sequence of claim 303, wherein the λN or a functional variant thereof comprises the amino acid sequence set forth in SEQ ID NO: 187 or 188.

305. The DNA sequence of claim 297 or claim 298, comprising a nucleic acid sequence selected from the group consisting of SEQ ID NOs: 62 and 150-156.

306. The DNA sequence of any one of claims 297-302, comprising the nucleic acid sequence set forth in SEQ ID NO: 151 or 152.

307. The DNA sequence of any one of claims 297-302, comprising a nucleic acid sequence encoding the amino acid sequence set forth in SEQ ID NO: 157 or 158.

308. The DNA sequence of any one of claims 297-302, comprising a nucleic acid sequence encoding the amino acid sequence set forth in SEQ ID NO: 192 or 193.

309. The DNA sequence of any one of claims 297-300, 303, and 304, comprising the nucleic acid sequence set forth in SEQ ID NO: 155 or 156.

310. The DNA sequence of any one of claims 297-300, 303, 304, and 309, comprising a nucleic acid sequence encoding the amino acid sequence set forth in SEQ ID NO: 161 or 162.

311. The DNA sequence of any one of claims 297-300, 303, 304, and 309, comprising a nucleic acid sequence encoding the amino acid sequence set forth in SEQ ID NO: 196 or 197.312.A DNA sequence encoding a viral matrix (MA) protein and a heterologous protein.

313. The DNA sequence of claim 312, which encodes a fusion protein comprising, from 5′ to 3′, the viral MA protein and the heterologous protein.

314. The DNA sequence of claim 312 or claim 313, wherein the encoded viral MA protein is derived from human immunodeficiency virus (HIV).

315. The DNA sequence of any of claims 312-314, wherein the encoded viral MA protein comprises the sequence set forth in SEQ ID NO:78.

316. The lipid particle of any of claims 1-201 and 262, the composition of claim 263, the method of any of claims 202-261 and 264-267, or the DNA sequence of any of claims 268-281 and 312-315, wherein the heterologous protein is a genome-modifying protein.

317. The lipid particle, composition, method, or DNA sequence of claim 316, wherein the genome-modifying protein comprises a recombinant nuclease, a nickase, an integrase, reverse transcriptase, or a combination thereof.

318. The lipid particle, composition, method, or DNA sequence of claim 316 or claim 317, wherein the genome-modifying protein comprises a zinc-finger nuclease (ZFN), a transcription-activator like effector nucleases (TALEN), or a CRISPR-associated (Cas) protein.

319. The lipid particle, composition, method, or DNA sequence of any of claims 316-318, wherein the genome-modifying protein is a Cas protein.

320. The lipid particle, composition, method, or DNA sequence of any of claims 316-319, wherein the genome-modifying protein is (i) Cas9, optionally saCas9 or spCas9; or (ii) cpf1.

321. The lipid particle of any of claims 1-201 and 262, the composition of claim 263, the method of any of claims 202-261 and 264-267, or the DNA sequence of any of claims 268-281 and 312-315, wherein the heterologous protein is a tumor neoepitope.

322. The lipid particle of any of claims 1-201 and 262, the composition of claim 263, the method of any of claims 202-261 and 264-267, or the DNA sequence of any of claims 268-281 and 312-315, wherein the heterologous protein is a viral Spike(s) glycoprotein.

323. The lipid particle of any of claims 1-201 and 262, the composition of claim 263, the method of any of claims 202-261 and 264-267, or the DNA sequence of any of claims 268-281 and 312-315, wherein the heterologous protein is a protein from Zika virus, optionally Zika virus prM-E protein; tuberculosis; respiratory syncytial virus (RSV), optionally RSV fusion (RSV-F) protein; influenza virus, optionally influenza virus hemagglutinin (HA); rabies virus, optionally rabies virus glycoprotein (RABV-G); human cytolomegalovirus (CMV); hepatitis C virus; human immunodeficiency virus 1 (HIV-1), and Streptococcus.

324. The lipid particle of any of claims 1-201 and 262, the composition of claim 263, the method of any of claims 202-261 and 264-267, or the DNA sequence of any of claims 268-281 and 312-315, wherein the heterologous protein is an antibody or an antigen-binding fragment thereof.

325. A vector comprising the DNA sequence of any of claims 268-324.

326. A mammalian cell comprising the DNA sequence of any of claims 268-324 or the vector of claim 325.

327. The mammalian cell of claim 326, further comprising viral nucleic acid, wherein the viral nucleic acid lacks one or more genes involved in viral replication.

328. The mammalian cell of claim 327, wherein the viral nucleic acid comprises:
one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3);
a nucleic acid encoding a viral envelope protein; and/or
a nucleic acid encoding a viral packaging protein selected from one or more of gag, pol, rev and tat.

329. The mammalian cell of any of claims 326-328, further comprising a RNA sequence encoding a heterologous protein.

330. The mammalian cell of any of claims 326-329, further comprising a guide RNA (gRNA).

331. A transfer plasmid comprising a promoter operably linked to a RNA sequence encoding a gag protein or portion thereof comprising at least a gag start codon; a RNA sequence encoding a heterologous protein that is linked to the RNA sequence encoding a gag protein or portion thereof; and a 3′ long terminal repeat (3′ LTR).

332. A transfer plasmid comprising a promoter operably linked to a ribonucleic acid (RNA), wherein the RNA comprises, from 5′ to 3′: a 5′ long terminal repeat (5′ LTR); a gag 5′ untranslated region (UTR) or portion thereof comprising at least three nucleotides; a RNA sequence encoding a heterologous protein that is linked to the gag 5′ UTR or a portion thereof; and a 3′ long terminal repeat (3′ LTR).

333. A transfer plasmid comprising a promoter operably linked to a ribonucleic acid (RNA), wherein the RNA comprises, from 5′ to 3′: a 5′ long terminal repeat (5′ LTR); a retroviral packaging sequence; a gag start codon; a RNA sequence encoding a heterologous protein; and a 3′ long terminal repeat (3′ LTR).

334. The transfer plasmid of claim 333, wherein the retroviral packaging sequence comprises a mutation in a major splice donor site.

335. The transfer plasmid of claim 334, wherein the major splice donor site is a major splice donor site contained in SL2 of HIV psi.

336. The transfer plasmid of claim 334 or claim 335, wherein the mutation is a mutation that inhibits splicing at the major splice donor site.

337. The transfer plasmid of any one of claims 334-336, wherein the mutated major splice donor site comprises a mutation that prevents splicing at the major splice donor site.

338. A transfer plasmid comprising a promoter operably linked to a nucleic acid sequence encoding a fusion protein comprising a viral matrix (MA) protein and a MS2 coat protein (MS2_{cp}).

339. A transfer plasmid comprising a promoter operably linked to a nucleic acid sequence encoding a viral matrix (MA) protein and a heterologous protein.

340. The transfer plasmid of any of claims 331-337, wherein the transfer plasmid is a lentiviral transfer plasmid.
